(19) United States
(12) Patent Application Publication
(10) Pub. No.: US 2004/0018560 A1
(43) Pub. Date: Jan. 29, 2004

(54) Crystallized LXR polypeptide in complex with a ligand and screening methods employing same

(76) Inventors: Randy K. Bledsoe, Durham, NC (US); Ann Miller, Wilmington, NC (US); John T. Moore, Durham, NC (US); Linda Moore, Durham, NC (US); Shawn P. Williams, Durham, NC (US); George B. Wisely, Durham, NC (US)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/418,007

(22) Filed: Apr. 17, 2003

Related U.S. Application Data
(60) Provisional application No. 60/376,019, filed on Apr. 26, 2002.

(51) Int. Cl. G01N 33/53; G06F 19/00; G01N 33/48; G01N 33/50; C07K 14/705

(52) U.S. Cl. 435/7.1; 530/350; 702/19

(57) ABSTRACT

Solved three-dimensional crystal structures of a human LXR ligand binding domain polypeptide in complex with one or more of a ligand and a coactivator polypeptide is disclosed. Contact points between the LXR and the ligands are also disclosed. Additionally, methods of designing modulators of the biological activity of LXR, and other LXR ligand binding domain polypeptides, are also disclosed.

### RELATED APPLICATIONS

This application claims priority to U.S. application No. 60/376,019 filed Apr. 26, 2002.

### TECHNICAL FIELD

The present invention relates generally to the structure of LXR, and more particularly to the crystalline structure of LXR in complex with a ligand. The invention further relates to methods by which modulators and ligands of a nuclear receptor, including a LXRβ can be identified.

Abbreviations

| | |
|---|---|
| APS | Advanced Photon Source |
| cDNA | complementary DNA |
| DBD | DNA binding domain |
| DMSO | dimethyl sulfoxide |
| DNA | deoxyribonucleic acid |
| DTT | dithiothreitol |
| EDTA | ethylenediaminetetraacetic acid |
| EPC | 24(S), 25-epoxycholesterol |
| FXR | farnesoid X receptor |
| HRE | hormone response element |
| kDa | kilodalton(s) |
| LBD | ligand binding domain |
| LN₂ | liquid nitrogen |
| LXR | liver X receptor |
| LXRα | liver X receptor α |
| LXRβ | liver X receptor β |
| NDP | nucleotide diphosphate |
| NR | nuclear receptor |
| nt | nucleotide |
| PCR | polymerase chain reaction |
| pl | isoelectric point |
| RMSD | root-mean-square deviation |
| SIRAS | single isomorphous replacement anomalous |
| | scattering |
| SPAP | secreted placental alkaline phosphatase |
| SRC1 | steroid receptor coactivator 1 |

Amino Acid Abbreviations

| Single-Letter Code | Three-Letter Code | Name |
|---|---|---|
| A | Ala | Alanine |
| V | Val | Valine |
| L | Leu | Leucine |
| I | Ile | Isoleucine |
| P | Pro | Proline |
| F | Phe | Phenylalanine |
| W | Trp | Tryptophan |
| M | Met | Methionine |
| G | Gly | Glycine |
| S | Ser | Serine |
| T | Thr | Threonine |
| C | Cys | Cysteine |
| Y | Tyr | Tyrosine |
| N | Asn | Asparagine |
| Q | Gln | Glutamine |
| D | Asp | Aspartic Acid |
| E | Glu | Glutamic Acid |
| K | Lys | Lysine |
| R | Arg | Arginine |
| H | His | Histidine |

Functionally Equivalent Codons

| | |
|---|---|
| Amino Acid | Codons |

| | | | |
|---|---|---|---|
| Alanine | Ala | A | GCA GCC GCG GCU |
| Cysteine | Cys | C | UGC UGU |
| Aspartic Acid | Asp | D | GAC GAU |
| Glumatic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Methionine | Met | M | AUG |
| Asparagine | Asn | N | AAC AAU |
| Proline | Pro | P | CCA CCC CCG CCU |
| Glutamine | Gln | Q | CAA CAG |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |
| Leucine | Leu | L | UUA UUG CUA CUC CUG CUU |
| Arginine | Arg | R | AGA AGG CGA CGC CGG CGU |
| Serine | Ser | S | ACG AGU UCA UCC UCG UCU |

### BACKGROUND ART

Nuclear receptors represent a superfamily of proteins that specifically bind a physiologically relevant small molecule, such as a hormone or vitamin. As a result of a molecule binding to a nuclear receptor, the nuclear receptor changes the ability of a cell to transcribe DNA, i.e. nuclear receptors modulate the transcription of DNA. However they can also have transcription independent actions.

Unlike integral membrane receptors and membrane-associated receptors, nuclear receptors reside in either the cytoplasm or nucleus of eukaryotic cells. Thus nuclear receptors comprise a class of intracellular, soluble ligand-regulated transcription factors. Nuclear receptors include but are not limited to receptors for glucocorticoids, androgens, mineralocorticoids, progestins, estrogens, thyroid hormones, vitamin D retinoids, and icosanoids. Many nuclear receptors, identified by either sequence homology to known receptors (See, Drewes et al., (1996) Mol. Cell. Biol. 16:925-31) or based on their affinity for specific DNA binding sites in gene promoters (See, Sladek et al., Genes Dev. 4:2353-65), have unascertained ligands and are therefore termed “orphan receptors.”

Structurally, nuclear receptors are generally characterized by two distinct structural elements. First, nuclear receptors comprise a central DNA binding domain that targets the receptor to specific DNA sequences, which are known as hormone response elements (HREs). The DNA binding domains of these receptors are related in structure and sequence, and are located within the middle of the receptor. Second, the C-terminal region of nuclear receptors encompasses the ligand binding domain (LBD). Upon binding a ligand, the receptor shifts to a transcriptionally active state.

Thus, the steroid/nuclear receptor superfamily is comprised of multi-domain transcription factors that regulate transcriptional activation in response to binding small molecules. The two liver X receptors, LXRα (NR1H3) and LXRβ (NR1H2), together with the farnesoid X receptor FXR (NR1H4) form a subset of the steroid/nuclear receptor superfamily that bind oxysterols (Janowski, et al., (1999) Proc. Nat. Acad. Sci. U.S.A. 96:266-271) and bile acids (Parks et al., (1999) Science 284:1365-1368; Makishima et al., (1999) Science 284:1362-1365), respectively. This subset of receptors is involved in the regulation of genes involved in cholesterol metabolism, transport, and elimination. LXR target genes include the ATP binding cassette ABCA1, SREBP1, etc. (Lehmann et al., (1997) J. Biol. Chem. 272:3137-3140; Schultz et al., (2000) Genes Dev. 14:2831-2838; Venkateswaran et al., (2000) Proc. Nat. Acad. Sci. U.S.A. 97:12097-12102). Oxysterols as natural LXR ligands is in accordance with their role in regulating cholesterol metabolism, and this family of compounds shows clear structure activity relationships with regard to LXR activation (Lehmann et al., (1997) J. Biol. Chem. 272:3137-3140; Forman et al., (1997) Proc. Nat. Acad. Sci. U.S.A. 94:10588-10593; Spencer et al., (2001) Journal of Med. Chem. 44:886-897). One of the most potent activators of both LXR subtypes is 24(S), 25-epoxycholesterol, which is widely found in the liver (Lehmann et al., (1997) J. Biol. Chem. 272: 3137-3140).

Because of the medical interest in cholesterol regulation, LXR has also been the target for drug discovery efforts. Several LXR specific compounds have been identified, including the compound N-(2,2,2-trifluoroethyl)-N-[4-(2,2,2-trifluoro-1-hydroxy-1-trifluoromethyl-ethyl)-phenyl]-benzenesulfonamide (also referred to interchangeably herein as “T0901317” and “T317”) (Schultz et al., (2000) Gene Dev. 14:2831-2838), a sulfonamide steroid mimetic that is as potent in activating both LXRs than the natural ligands. These compounds have been shown to be effective in vivo in reducing serum cholesterol levels and plaque accumulation.

Unraveling the structural basis of how LXR recognizes an array of different endogenous and exogenous compounds, including both small and large ligands, is critical to understanding how compounds, such as cholesterol, are cleared from the body. Such knowledge can also improve the ability to predict and avoid dangerous drug-drug interactions.

Polypeptides, including the ligand binding domain of LXRα and the ligand binding domain of LXRβ, have a three-dimensional structure determined by the primary amino acid sequence and the environment surrounding the polypeptide. This three-dimensional structure establishes the polypeptide's activity, stability, binding affinity, binding specificity, and other biochemical attributes. Thus, knowledge of a protein's three-dimensional structure can provide much guidance in designing agents that mimic, inhibit, or improve its biological activity in soluble or membrane bound forms.

The three-dimensional structure of a polypeptide can be determined in a number of ways. Many of the most precise methods employ X-ray crystallography (See, e.g., Van Holde, (1971) Physical Biochemistry, Prentice-Hall, New Jersey, pp. 221-39). This technique relies on the ability of crystalline lattices to diffract X-rays or other forms of radiation. Diffraction experiments suitable for determining the three-dimensional structure of macromolecules typically require high-quality crystals. Unfortunately, such crystals have been unavailable for the ligand binding domain of LXR as well as many other proteins of interest. Thus, high-quality diffracting crystals of the ligand binding domain of LXR would greatly assist in the elucidation of LXR's three-dimensional structure, and would provide insight into the ligand binding properties of LXR.

Clearly, the solved crystal structure of the LXR ligand binding domain would be useful in the design of modulators of activity mediated by all LXR isoforms. Additionally, evaluation of the available sequence data has made it clear that LXR shows structural homology with the three-dimensional fold of other proteins.

A solved LXR-ligand crystal structure would provide structural details and insights necessary to design a modulator of LXR that maximizes preferred requirements for any modulator, i.e. potency and specificity. A LXR-ligand-coactivator fragment crystal structure would provide additional information. By exploiting the structural details obtained from a LXR-ligand crystal structure, or a LXR-ligand-coactivator fragment crystal structure, it would be possible to design a LXR modulator that, despite LXR's similarity with other proteins, exploits the unique structural features of LXR. A LXR modulator developed using structure-assisted design would take advantage of heretofore unknown LXR structural considerations and thus be more effective than a modulator developed using homology-based design. Potential or existent homology models cannot provide the necessary degree of specificity. A LXR modulator designed using the structural coordinates of a crystalline form of LXR would also provide a starting point for the development of modulators of other structurally similar proteins.

What is needed, therefore, is a crystallized form of a LXR LBD polypeptide, preferably in complex with a ligand. Acquisition of crystals of the LXR LBD polypeptide will permit the three dimensional structure of the LXR LBD to be determined. Knowledge of this three dimensional structure will facilitate the design of modulators of LXR activity, as well as the activity of other NRs. Such modulators can lead to therapeutic compounds to treat a wide range of conditions, including conditions associated with high serum cholesterol levels.

### SUMMARY OF THE INVENTION

A crystalline form comprising a substantially pure LXR ligand binding domain polypeptide and a ligand is disclosed. Preferably, the crystalline form has lattice constants of a=60.25 Å, b=82.45 Å, c=123.18 Å, α=90°, β=90°, γ=90°. More preferably, the crystalline form is an orthogonal crystalline form. Even more preferably, the crystalline form has a space group of P2₁2₁2. Still more preferably, the LXR ligand binding domain polypeptide has the amino acid sequence shown in SEQ ID NO: 8. It is also preferable that the ligand is N-(2,2,2-trifluoroethyl)-N-[4-(2,2,2-trifluoro-1-hydroxy-1-trifluoromethyl-ethyl)-phenyl]-benzenesulfonamide.

Yet another crystalline form comprising a substantially pure LXR ligand binding domain polypeptide, a ligand and a coactivator polypeptide is disclosed. Preferably, the crystalline form has lattice constants of a=71.17 Å, b=120.01 Å, c=147.56 Å, α=90°, β=90°, γ=90°. More preferably, the crystalline form is an orthoganol crystalline form. Even more preferably, the crystalline form has a space group of C222₁. Still more preferably, the LXR ligand binding domain polypeptide has the amino acid sequence shown in SEQ ID NO: 8. It is also preferable that the ligand is is 24(S), 25-epoxycholesterol and the coactivator polypeptide comprises a SRC1 polypeptide comprising the sequence of SEQ ID NO: 9.

A method for determining the three-dimensional structure of a crystallized LXR ligand binding domain polypeptide in complex with one or more of a ligand and a coactivator polypeptide to a resolution of about 2.8 Å or better is disclosed. In a preferred embodiment, the method comprises: (a) crystallizing a LXR ligand binding domain polypeptide in complex with one or more of a ligand and a coactivator polypeptide to form a crystallized complex; and (b) analyzing the crystallized complex to determine the three-dimensional structure of the LXR ligand binding domain polypeptide in complex with one or more of a ligand and a coactivator polypeptide, whereby the three-dimensional structure of a crystallized LXR ligand binding domain polypeptide in complex with one or more of a ligand and a coactivator polypeptide is determined to a resolution of about 2.8 Å or better. It is also preferable that the ligand is selected from the group consisting of 24(S), 25-epoxycholesterol and N-(2,2,2-trifluoroethyl)-N-[4-(2,2,2-trifluoro-1-hydroxy-1-trifluoromethyl-ethyl)-phenyl]-benzenesulfonamide and that the coactivator polypeptide comprises a SRCI polypeptide comprising the sequence of SEQ ID NO: 9.

A method of generating a crystalline form comprising a LXR ligand binding domain polypeptide in complex with one or more of a ligand and a coactivator polypeptide is disclosed. In a preferred embodiment, the method comprises: (a) incubating a solution comprising a LXR ligand binding domain and one or more of a ligand and a coactivator polypeptide with an equal volume of reservoir; and (b) crystallizing the LXR ligand binding domain polypeptide and one or more of a ligand and a coactivator polypeptide using the hanging drop method, whereby a crystalline form of a LXR ligand binding domain polypeptide in complex with one or more of a ligand and a coactivator polypeptide is generated. It is also preferable that the ligand is selected from the group consisting of 24(S), 25-epoxycholesterol and N-(2,2,2-trifluoroethyl)-N-[4-(2,2,2-trifluoro-1-hydroxy-1-trifluoromethyl-ethyl)-phenyl]-benzenesulfonamide and that the coactivator polypeptide comprises a SRC1 polypeptide comprising the sequence of SEQ ID NO: 9.

A method of designing a modulator of a NR polypeptide is disclosed. In a preferred embodiment, the method comprises: (a) designing a potential modulator of a NR polypeptide that will make interactions with amino acids in a ligand binding site of the NR polypeptide, based upon a crystalline structure comprising a LXR ligand binding domain polypeptide in complex with a one or more of a ligand and a coactivator polypeptide; (b) synthesizing the modulator; and (c) determining whether the potential modulator modulates the activity of the NR polypeptide, whereby a modulator of a NR polypeptide is designed. It is preferable that the NR comprises a LXR and more preferably a LXRβ comprising the sequence of SEQ ID NO: 4. It is also preferable that the ligand is selected from the group consisting of 24(S), 25-epoxycholesterol and N-(2,2,2-trifluoroethyl)-N-[4-(2,2,2-trifluoro-1-hydroxy-1-trifluoromethyl-ethyl)-phenyl]-benzenesulfonamide and that the coactivator polypeptide comprises a SRC1 polypeptide comprising the sequence of SEQ ID NO: 9.

In an alternative embodiment, a method of designing a modulator that selectively modulates the activity of a NR polypeptide in accordance with the present invention comprises: (a) obtaining a crystalline form comprising a LXRβ ligand binding domain polypeptide in complex with one or more of a ligand and a coativator polypeptide; (b) evaluating the three-dimensional structure of the crystallized LXRβ ligand binding domain polypeptide in complex with one or more of a ligand and a coactivator polypeptide; and (c) synthesizing a potential modulator based on the three-dimensional structure of the crystallized LXRβ ligand binding domain polypeptide in complex with one or more of a ligand and a coactivator polypeptide, whereby a modulator that selectively modulates the activity of a NR polypeptide is designed. Preferably, the method further comprises contacting a NR ligand binding domain polypeptide with the potential modulator and one or more of a ligand and a coactivator polypeptide; and assaying the NR ligand binding domain polypeptide for binding of the potential modulator, for a change in activity of the NR ligand binding domain polypeptide, or both. In this embodiment, it is preferable that the NR comprises a LXR and more preferably a LXRβ comprising the sequence of SEQ ID NO: 4. It is also preferable that the ligand is selected from the group consisting of 24(S), 25-epoxycholesterol and N-(2,2,2-trifluoroethyl)-N-[4-(2,2,2-trifluoro-1-hydroxy-1-trifluoromethyl-ethyl)-phenyl]-benzenesulfonamide and that the coactivator polypeptide comprises a SRC1 polypeptide comprising the sequence of SEQ ID NO: 9.

A method of screening a plurality of compounds for a modulator of a NR ligand binding domain polypeptide is also disclosed. In a preferred embodiment, the method comprises: (a) providing a library of test samples; (b) contacting a crystalline form comprising a LXR ligand binding domain polypeptide in complex with one or more of a ligand and a coactivator polypeptide with each test sample; (c) detecting an interaction between a test sample and the crystalline LXR ligand binding domain polypeptide in complex with one or more of a ligand and a coactivator polypeptide; (d) identifying a test sample that interacts with the crystalline LXR ligand binding domain polypeptide in complex with one or more of a ligand and a coactivator polypeptide; and (e) isolating a test sample that interacts with the crystalline LXR ligand binding domain polypeptide in complex with one or more of a ligand and a coregulator polypeptide, whereby a plurality of compounds is screened for a modulator of a NR ligand binding domain polypeptide. In this embodiment, it is preferable that the NR comprises a LXR and more preferably a LXRβ comprising the sequence of SEQ ID NO: 4. It is also preferable that the ligand is selected from the group consisting of 24(S), 25-epoxycholesterol and N-(2,2,2-trifluoroethyl)-N-[4-(2,2,2-trifluoro-1-hydroxy-1-trifluoromethyl-ethyl)-phenyl]-benzenesulfonamide and that the coactivator polypeptide comprises a SRCI polypeptide comprising the sequence of SEQ ID NO: 9.

Additionally, a method for identifying a NR modulator is disclosed. IN a preferred embodiment, the method comprises: (a) providing atomic coordinates of a LXR ligand binding domain in complex with one or more of a ligand and a coactivator polypeptide to a computerized modeling system; and (b) modeling ligands that fit spatially into the binding pocket of the LXR ligand binding domain to thereby identify a NR modulator. Preferably the method further comprises identifying in an assay for NR-mediated activity a modeled ligand that increases or decreases the activity of the NR. In this embodiment, it is preferable that the NR comprises a LXR and more preferably a LXRβ comprising the sequence of SEQ ID NO: 4. It is also preferable that the ligand is selected from the group consisting of 24(S), 25-epoxycholesterol and N-(2,2,2-trifluoroethyl)-N-[4-(2,2,2-trifluoro-1-hydroxy-1-trifluoromethyl-ethyl)-phenyl]-benzenesulfonamide and that the coactivator polypeptide comprises a SRC1 polypeptide comprising the sequence of SEQ ID NO: 9.

In another embodiment of the present invention, a method of identifying a LXR modulator that selectively modulates the activity of a LXRβ polypeptide compared to other polypeptides is disclosed. In a preferred embodiment, the method comprises: (a) providing atomic coordinates of a LXRβ ligand binding domain in complex with one or more of a ligand and a coactivator polypeptide to a computerized modeling system; and (b) modeling a ligand that fits into the binding pocket of a LXRβ ligand binding domain and that interacts with residues of a LXRβ that are conserved among LXR subtypes to thereby identify a LXRβ modulator that selectively modulates the activity of a LXRβ polypeptide compared to other polypeptides. In this embodiment, it is preferable that the LXRβ comprises the sequence of SEQ ID NO: 4. It is also preferable that the ligand is selected from the group consisting of 24(S), 25-epoxycholesterol and N-(2,2,2-trifluoroethyl)-N-[4-(2,2,2-trifluoro-1-hydroxy-1-trifluoromethyl-ethyl)-phenyl]-benzenesulfonamide and that the coactivator polypeptide comprises a SRC1 polypeptide comprising the sequence of SEQ ID NO: 9. It is more preferable that the method further comprises identifying in a biological assay for LXRβ activity a modeled ligand that selectively binds to said LXRβ and increases or decreases the activity of said LXRβ.

In yet another embodiment, a method of designing a modulator of a NR polypeptide is disclosed. In a preferred embodiment, the method comprises: (a) selecting a candidate NR ligand; (b) determining which amino acid or amino acids of a NR polypeptide interact with the ligand using a three-dimensional model of a crystallized protein, the model comprising a LXRβ ligand binding domain in complex with one or more of a ligand and a coactivator polypeptide; (c) identifying in a biological assay for NR activity a degree to which the ligand modulates the activity of the NR polypeptide; (d) selecting a chemical modification of the ligand wherein the interaction between the amino acids of the NR polypeptide and the ligand is predicted to be modulated by the chemical modification; (e) synthesizing a ligand having the chemical modified to form a modified ligand; (f) contacting the modified ligand with the NR polypeptide; (g) identifying in a biological assay for NR activity a degree to which the modified ligand modulates the biological activity of the NR polypeptide; and (h) comparing the biological activity of the NR polypeptide in the presence of modified ligand with the biological activity of the NR polypeptide in the presence of the unmodified ligand, whereby a modulator of a NR polypeptide is designed. In this embodiment, it is preferable that the NR comprises a LXR and more preferably a LXRβ comprising the sequence of SEQ ID NO: 4. It is also preferable that the ligand is selected from the group consisting of 24(S), 25-epoxycholesterol and N-(2,2,2-trifluoroethyl)-N-[4-(2,2,2-trifluoro-1-hydroxy-1-trifluoromethyl-ethyl)-phenyl]-benzenesulfonamide and that the coactivator polypeptide comprises a SRC1 polypeptide comprising the sequence of SEQ ID NO: 9. It is additionally preferable that the the method further comprises repeating steps (a) through (f), if the biological activity of the NR polypeptide in the presence of the modified ligand varies from the biological activity of the NR polypeptide in the presence of the unmodified ligand.

Further, a method of identifying a LXR modulator that selectively modulates the biological activity of one LXR subtype compared to LXRβ is disclosed. In a preferred embodiment, the method comprises: (a) providing an atomic structure coordinate set describing a LXRβ ligand binding domain structure in complex with one or more of a ligand and a coactivator polypeptide and at least one other atomic structure coordinate set describing a LXR ligand binding domain, each ligand binding domain comprising a ligand binding site; (b) comparing the LXR atomic structure coordinate sets to identify at least one difference between the sets; (c) designing a candidate ligand predicted to interact with the difference of step (b); (d) synthesizing the candidate ligand; and (e) testing the synthesized candidate ligand for an ability to selectively modulate a LXR subtype as compared to LXRβ, whereby a LXR modulator that selectively modulates the biological activity of one LXR subtype compared to LXRβ is identified. In this embodiment, it is preferable that the LXR subtype whose biological activity is to be modulated is LXRβ and comprises the sequence of SEQ ID NO: 4. It is also preferable that the ligand is selected from the group consisting of 24(S), 25-epoxycholesterol and N-(2,2,2-trifluoroethyl)-N-[4-(2,2,2-trifluoro-1-hydroxy-1-trifluoromethyl-ethyl)-phenyl]-benzenesulfonamide and that the coactivator polypeptide comprises a SRC1 polypeptide comprising the sequence of SEQ ID NO: 9.

In another embodiment, a method of modeling a three-dimensional structure of a target NR in complex with one or more of a ligand and a coactivator polypeptide from a template comprising the X-ray structure of a LXR in complex with one or more of a ligand and a coactivator polypeptide is disclosed. In a preferred embodiment, the method comprises: (a) selecting an X-ray structure of a target NR LBD as a starting model for the target NR LBD; (b) manipulating the starting model for the target NR as a rigid body to superimpose its backbone atoms onto corresponding backbone atoms of a three-dimensional template structure comprising a LXR in complex with one or more of a ligand and a coactivator polypeptide to form a manipulated model; (c) making a copy of the coactivator peptide from the template structure to form a model of a coactivator polypeptide bound to a template LXR; (d) merging the model of the coactivator polypeptide into the manipulated model to form a modified model; (e) removing one or more amino acids from the modified model; and (f) optimizing side-chain conformations, whereby a three-dimensional structure of a target NR in complex with one or more of a ligand and a coactivator polypeptide is modeled from a template comprising the X-ray structure of an NR in complex with one or more of a ligand and a coactivator polypeptide. In this embodiment, it is preferable that the LXR template is LXRβ and comprises the sequence of SEQ ID NO: 4. It is also preferable that the ligand is selected from the group consisting of 24(S), 25-epoxycholesterol and N-(2,2,2-trifluoroethyl)-N-[4-(2,2,2-trifluoro-1-hydroxy-1-trifluoromethyl-ethyl)-phenyl]-benzenesulfonamide and that the coactivator polypeptide comprises a SRC1 polypeptide comprising the sequence of SEQ ID NO: 9.

Accordingly, it is an object of the present invention to provide a three dimensional structure of the ligand binding domain of an LXR in complex with one or more of a ligand and a coactivator polypeptide. The object is achieved in whole or in part by the present invention.

An object of the invention having been stated hereinabove, other objects will be evident as the description proceeds, when taken in connection with the accompanying Drawings and Laboratory Examples as best described hereinbelow.

### BRIEF DESCRIPTION OF THE DRAWINGS

FIG. 1 is a ribbon diagram depicting the LXRβ/EPC/SRC1 dimer that formed during crystallization. LXR is gray, SRC1 is black, and EPC is light gray. The LXR dimer formed is the same in both compound complexes.

FIG. 2 is a ribbon diagram depicting an EPC molecule situated in the LXRβ binding pocket. EPC is a light gray ball and stick figure with darker oxygen atoms.

FIG. 3 is a diagram depicting residues that interact with EPC in the LXRβ binding pocket; EPC is drawn as a ball and stick figure with gray carbons and black oxygens and LXRβ is depicted as a wire frame model.

FIG. 4A is a ribbon diagram depicting the superimposition of Cα atoms of each member of the crystallized LXRβ dimer, as well as the superimposition of the ligand N-(2,2,2-trifluoroethyl)-N-[4-(2,2,2-trifluoro-1-hydroxy-1-trifluoromethyl-ethyl)-phenyl]-benzenesulfonamide, one molecule of which was associated with each of the LXRβ monomers. The A molecule is indicated with light ribbon, while the B molecule is indicated with darker ribbon.

FIG. 4B is a ribbon diagram depicting the position of the ligand N-(2,2,2-trifluoroethyl)-N-[4-(2,2,2-trifluoro-1-hydroxy-1-trifluoromethyl-ethyl)-phenyl]-benzenesulfonamide (T317) relative to helices 5, 7 and 11 of LXRβ. In this figure the superimposition of Cα atoms of each member of the crystallized LXRβ dimer, as well as a detailed view of the superimposition of the ligand, one molecule of which was associated with each of the LXRβ monomers is represented. The A molecules of both LXRβ and T317 are light colored, while the B molecules are darker.

FIG. 5 is a diagram depicting the superimposition of each LXR of the dimer (wire frame model) and the superimposition of the N-(2,2,2-trifluoroethyl)-N-[4-(2,2,2-trifluoro-1-hydroxy-1-trifluoromethyl-ethyl)-phenyl]-benzenesulfonamide (T317) molecule associated with each of the LXRs (ball and stick). The A molecules of LXRβ and T317 are light colored, while the B molecules are darker.

FIG. 6A is a ribbon diagram of the binding pocket of LXR demonstrating how the structure was used to identify amino acids specifically contacting EPC to design mutants. In this figure the EPC A ring extends beyond T317 and O3 and reaches into a polar pocket containing E281, E315, and R319.

FIG. 6B is a ribbon diagram of the binding pocket of LXR demonstrating how the structure was used to identify amino acids specifically contacting T317 to design mutants. In this figure the ligand T317 is shown to form a hydrogen bond with H435 and extends into a hydrophobic pocket along the beta turn containing 1327, F329, and Y335.

FIG. 7A is a bar graph depicting the effect of mutating LXR LBD pocket residues on transcriptional activity, which was determined by measuring the amount of SPAP synthesized in CV-1 cells by a transiently transfected Gal4 fusion of LXRα LBD.

FIG. 7B is a bar graph depicting the effect of mutating LXR LBD pocket residues on transcriptional activity, which was determined by measuring the amount of SPAP synthesized in CV-1 cells by a transiently transfected Gal4 fusion of LXRβ LBD.

### BRIEF DESCRIPTION OF THE SEQUENCES IN THE SEQUENCE LISTING

SEQ ID NO: 1 is a DNA sequence encoding a full-length human LXRα polypeptide (Swiss-Prot Accession No. Q13133; GenBank Accession No. U22662).

SEQ ID NO: 2 is an amino acid sequence comprising a full-length human LXRα (Swiss-Prot Accession No. Q13133; GenBank Accession No. U22662).

SEQ ID NO: 3 is a DNA sequence encoding a full-length human LXRβ polypeptide (Swiss-Prot Accession No. P55055; GenBank Accession No. NM_{—}007121).

SEQ ID NO: 4 is an amino acid sequence comprising a full-length human LXRβ (Swiss-Prot Accession No. P55055; GenBank Accession No. NM_{—}007121).

SEQ ID NO: 5 is a DNA sequence encoding a human LXRα ligand binding domain polypeptide (Swiss-Prot Accession No. Q13133; GenBank Accession No. U22662).

SEQ ID NO: 6 is an amino acid sequence comprising a human LXRα ligand binding domain polypeptide (Swiss-Prot Accession No. Q13133; GenBank Accession No. U22662).

SEQ ID NO: 7 is a DNA sequence encoding a human LXRβ ligand binding domain polypeptide.

SEQ ID NO: 8 is an amino acid sequence comprising a human LXRβ ligand binding domain polypeptide.

SEQ ID NO: 9 is an amino acid sequence that comprises a fragment of the co-activator SRC1.

SEQ ID NO: 10 is an amino acid sequence of residues 182-419 of an RAR polypeptide that was employed in the molecular replacement solution of LXRβ.

### DETAILED DESCRIPTION OF THE INVENTION

Until disclosure of the present invention presented herein, the ability to obtain crystalline forms of a LXRβ LBD has not been realized. And until disclosure of the present invention presented herein, a detailed three-dimensional crystal structure of a LXRβ polypeptide has not been solved.

In addition to providing structural information, crystalline polypeptides provide other advantages. For example, the crystallization process itself further purifies the polypeptide, and satisfies one of the classical criteria for homogeneity. In fact, crystallization frequently provides unparalleled purification quality, removing impurities that are not removed by other purification methods such as HPLC, dialysis, conventional column chromatography, etc. Moreover, crystalline polypeptides are often stable at ambient temperatures and free of protease contamination and other degradation associated with solution storage. Crystalline polypeptides can also be useful as pharmaceutical preparations. Finally, crystallization techniques in general are largely free of problems such as denaturation associated with other stabilization methods (e.g., lyophilization). Once crystallization has been accomplished, crystallographic data provides useful structural information that can assist the design of compounds that can serve as agonists or antagonists, as described herein below. In addition, the crystal structure provides information useful to map a receptor-binding domain, which could then be mimicked by a small non-peptide molecule that would serve as an antagonist or agonist.

I. Definitions

Following long-standing patent law convention, the terms “a” and “an” mean “one or more” when used in this application, including the claims.

As used herein, the term “mutation” carries its traditional connotation and means a change, inherited, naturally occurring or introduced, in a nucleic acid or polypeptide sequence, and is used in its sense as generally known to those of skill in the art.

As used herein, the term “labeled” means the attachment of a moiety, capable of detection by spectroscopic, radiologic or other methods, to a probe molecule.

As used herein, the term “target cell” refers to a cell, into which it is desired to insert a nucleic acid sequence or polypeptide, or to otherwise effect a modification from conditions known to be standard in the unmodified cell. A nucleic acid sequence introduced into a target cell can be of variable length. Additionally, a nucleic acid sequence can enter a target cell as a component of a plasmid or other vector or as a naked sequence.

As used herein, the term “transcription” means a cellular process involving the interaction of an RNA polymerase with a gene that directs the expression as RNA of the structural information present in the coding sequences of the gene. The process includes, but is not limited to the following steps: (a) the transcription initiation, (b) transcript elongation, (c) transcript splicing, (d) transcript capping, (e) transcript termination, (f) transcript polyadenylation, (g) nuclear export of the transcript, (h) transcript editing, and (i) stabilizing the transcript.

As used herein, the term “expression” generally refers to the cellular processes by which a polypeptide is produced from RNA.

As used herein, the term “transcription factor” means a cytoplasmic or nuclear protein which binds to a gene, or binds to an RNA transcript of a gene, or binds to another protein which binds to a gene or an RNA transcript or another protein which in turn binds to a gene or an RNA transcript, so as to thereby modulate expression of the gene. Such modulation can additionally be achieved by other mechanisms; the essence of a “transcription factor for a gene” pertains to a factor that alters the level of transcription of the gene in some way.

As used herein, the term “hybridization” means the binding of a probe molecule, a molecule to which a detectable moiety has been bound, to a target sample.

As used herein, the term “detecting” means confirming the presence of a target entity by observing the occurrence of a detectable signal, such as a radiologic or spectroscopic signal that will appear exclusively in the presence of the target entity.

As used herein, the term “sequencing” means determining the ordered linear sequence of nucleic acids or amino acids of a DNA or protein target sample, using conventional manual or automated laboratory techniques.

As used herein, the term “isolated” means oligonucleotides substantially free of other nucleic acids, proteins, lipids, carbohydrates or other materials with which they can be associated, such association being either in cellular material or in a synthesis medium. The term can also be applied to polypeptides, in which case the polypeptide will be substantially free of nucleic acids, carbohydrates, lipids and other undesired polypeptides.

As used herein, the term “substantially pure” means that the polynucleotide or polypeptide is substantially free of the sequences and molecules with which it is associated in its natural state, and those molecules used in the isolation procedure. The term “substantially free” means that the sample is at least 50%, preferably at least 70%, more preferably 80% and most preferably 90% free of the materials and compounds with which is it associated in nature.

As used herein, the term “primer” means a sequence comprising two or more deoxyribonucleotides or ribonucleotides, preferably more than three, and more preferably more than eight and most preferably at least about 20 nucleotides of an exonic or intronic region. Such oligonucleotides are preferably between ten and thirty bases in length.

As used herein, the term “DNA segment” means a DNA molecule that has been isolated free of total genomic DNA of a particular species. In a preferred embodiment, a DNA segment encoding a LXR polypeptide refers to a DNA segment that comprises SEQ ID NOs: 1, 3, 5, and 7, but can optionally comprise fewer or additional nucleic acids, yet is isolated away from, or purified free from, total genomic DNA of a source species, such as Homo sapiens. Included within the term “DNA segment” are DNA segments and smaller fragments of such segments, and also recombinant vectors, including, for example, plasmids, cosmids, phages, viruses, and the like.

As used herein, the phrase “enhancer-promoter” means a composite unit that contains both enhancer and promoter elements. An enhancer-promoter is operatively linked to a coding sequence that encodes at least one gene product.

As used herein, the phrase “operatively linked” means that an enhancer-promoter is connected to a coding sequence in such a way that the transcription of that coding sequence is controlled and regulated by that enhancer-promoter. Techniques for operatively linking an enhancer-promoter to a coding sequence are well known in the art; the precise orientation and location relative to a coding sequence of interest is dependent, inter alia, upon the specific nature of the enhancer-promoter.

As used herein, the terms “candidate substance” and “candidate compound” are used interchangeably and refer to a substance that is believed to interact with another moiety, for example a given ligand that is believed to interact with a complete, or a fragment of, a LXR polypeptide, and which can be subsequently evaluated for such an interaction. Representative candidate substances or compounds include “xenobiotics”, such as drugs and other therapeutic agents, carcinogens and environmental pollutants, natural products and extracts, as well as “endobiotics”, such as steroids, fatty acids and prostaglandins. Other examples of candidate compounds that can be investigated using the methods of the present invention include, but are not restricted to, agonists and antagonists of a LXR polypeptide, toxins and venoms, viral epitopes, hormones (e.g., opioid peptides, steroids, etc.), hormone receptors, peptides, enzymes, enzyme substrates, co-factors, lectins, sugars, oligonucleotides or nucleic acids, oligosaccharides, proteins, small molecules and monoclonal antibodies.

As used herein, the term “biological activity” means any observable effect flowing from interaction between a LXR polypeptide and a ligand. Representative, but non-limiting, examples of biological activity in the context of the present invention include dimerization of a LXR polypeptide and association of a LXR with a ligand, such as 24(S), 25-epoxycholesterol.

As used herein, the term “modified” means an alteration from an entity's normally occurring state. An entity can be modified by removing discrete chemical units or by adding discrete chemical units. The term “modified” encompasses detectable labels as well as those entities added as aids in purification.

As used herein, the terms “structure coordinates” and “structural coordinates” mean mathematical coordinates derived from mathematical equations related to the patterns obtained on diffraction of a monochromatic beam of X-rays by the atoms (scattering centers) of a molecule in crystal form. The diffraction data are used to calculate an electron density map of the repeating unit of the crystal. The electron density maps are used to establish the positions of the individual atoms within the unit cell of the crystal.

Those of skill in the art understand that a set of structure coordinates determined by X-ray crystallography is not without standard error. For the purpose of this invention, any set of structure coordinates for LXR or a LXR mutant that have a root mean square deviation (RMSD) from ideal of preferably no more than 1.5 Å, more preferably no more than 1.0 Å, and most preferably no more than 0.5 Å when superimposed, using the polypeptide backbone atoms, on the structure coordinates listed in Tables 2, and 3 shall be considered identical.

As used herein, the term “space group” means the arrangement of symmetry elements of a crystal.

As used herein, the term “molecular replacement” means a method that involves generating a preliminary model of the wild-type LXR ligand binding domain, or a LXR mutant crystal whose structure coordinates are unknown, by orienting and positioning a molecule whose structure coordinates are known within the unit cell of the unknown crystal so as best to account for the observed diffraction pattern of the unknown crystal. Phases can then be calculated from this model and combined with the observed amplitudes to give an approximate Fourier synthesis of the structure whose coordinates are unknown. This, in turn, can be subject to any of the several forms of refinement to provide a final, accurate structure of the unknown crystal. See, e.g., Lattman, (1985) Method Enzymol. 115: 55-77; Rossmann (ed.), (1972) The Molecular Replacement Method, Gordon & Breach, New York. Using the structure coordinates of a LXR ligand binding domain provided by the present invention, molecular replacement can be used to determine the structure coordinates of a crystalline mutant or homologue of a LXR ligand binding domain, or of a different crystal form of the LXR ligand binding domain.

As used herein, the term “isomorphous replacement” means a method of using heavy atom derivative crystals to obtain the phase information necessary to elucidate the three-dimensional structure of a native crystal (Blundell et al., (1976) Protein Crystallography, Academic Press; Otwinowski, (1991) in Isomorphous Replacement and Anomalous Scattering, (Evans & Leslie, eds.), 80-86, Daresbury Laboratory, Daresbury, United Kingdom). The phrase “heavy-atom derivatization” is synonymous with the term “isomorphous replacement.”

As used herein, the terms “β-sheet” and “beta-sheet” mean the conformation of a polypeptide chain stretched into an extended zig-zig conformation. Portions of polypeptide chains that run “parallel” all run in the same direction. Polypeptide chains that are “antiparallel” run in the opposite direction from the parallel chains.

As used herein, the terms “α-helix” and “alpha-helix” mean the conformation of a polypeptide chain wherein the polypeptide backbone is wound around the long axis of the molecule in a left-handed or right-handed direction, and the R groups of the amino acids protrude outward from the helical backbone, wherein the repeating unit of the structure is a single turnoff the helix, which extends about 0.56 nm along the long axis.

As used herein, the term “unit cell” means a basic parallelepiped shaped block. The entire volume of a crystal can be constructed by regular assembly of such blocks. Each unit cell comprises a complete representation of the unit of pattern, the repetition of which builds up the crystal. Thus, the term “unit cell” means the fundamental portion of a crystal structure that is repeated infinitely by translation in three dimensions. A unit cell is characterized by three vectors a, b, and c, not located in one plane, which form the edges of a parallelepiped. Angles α, β and γ define the angles between the vectors: angle α is the angle between vectors b and c; angle β is the angle between vectors a and c; and angle γ is the angle between vectors a and b. The entire volume of a crystal can be constructed by regular assembly of unit cells; each unit cell comprises a complete representation of the unit of pattern, the repetition of which builds up the crystal.

As used herein, the term “orthogonal unit cell” means a unit cell wherein a≠b≠c; and α=β=γ=90°. The vectors a, b and c describe the unit cell edges and the angles α, β, and γ describe the unit cell angles.

As used herein, the term “crystal lattice” means the array of points defined by the vertices of packed unit cells.

As used herein, the term “ligand binding site” and “ligand binding domain” are used interchangeably and mean that site in a polypeptide where substrate binding occurs. For human LXRβ, the ligand binding domain comprises the residues 214-462 of the full-length human LXRβ protein.

As used herein, the term “LXR” means nucleic acids encoding a liver X receptor (LXR) nuclear receptor polypeptide that can bind DNA and/or one or more ligands, and/or has the ability to form multimers. The term “LXR” includes invertebrate homologs; however, preferably, LXR nucleic acids and polypeptides are isolated from vertebrate sources. “LXR” further includes vertebrate homologs of LXR family members, including, but not limited to, mammalian and avian homologs. Representative mammalian homologs of LXR family members include, but are not limited to, murine and human homologs.

As used herein, the terms “LXR gene product”, “LXR protein”, “LXR polypeptide”, and “LXR peptide” are used interchangeably and mean peptides having amino acid sequences which are substantially identical to native amino acid sequences from an organism of interest and which are biologically active in that they comprise all or a part of the amino acid sequence of a LXR polypeptide, or cross-react with antibodies raised against a LXR polypeptide, or retain all or some of the biological activity (e.g., DNA or ligand binding ability and/or dimerization ability) of the native amino acid sequence or protein. Such biological activity can include immunogenicity.

As used herein, the terms “LXR gene product”, “LXR protein”, “LXR polypeptide”, and “LXR peptide” also include analogs of a LXR polypeptide. By “analog” is intended that a DNA or peptide sequence can contain alterations relative to the sequences disclosed herein, yet retain all or some of the biological activity of those sequences. Analogs can be derived from genomic nucleotide sequences as are disclosed herein or from other organisms, or can be created synthetically. Those skilled in the art will appreciate that other analogs, as yet undisclosed or undiscovered, can be used to design and/or construct LXR analogs. There is no need for a “LXR gene product”, “LXR protein”, “LXR polypeptide”, or “LXR peptide” to comprise all or substantially all of the amino acid sequence of a LXR polypeptide gene product. Shorter or longer sequences are anticipated to be of use in the invention; shorter sequences are herein referred to as “segments”. Thus, the terms “LXR gene product”, “LXR protein”, “LXR polypeptide”, and “LXR peptide” also include fusion, chimeric or recombinant LXR polypeptides and proteins comprising sequences of the present invention. Methods of preparing such proteins are disclosed herein and are known in the art.

As used herein, the term “polypeptide” means any polymer comprising any of the 20 protein amino acids, regardless of its size. Although “protein” is often used in reference to relatively large polypeptides, and “peptide” is often used in reference to small polypeptides, usage of these terms in the art overlaps and varies. The term “polypeptide” as used herein refers to peptides, polypeptides and proteins, unless otherwise noted. As used herein, the terms “protein”, “polypeptide” and “peptide” are used interchangeably herein when referring to a gene product.

As used herein, the term “modulate” means an increase, decrease, or other alteration of any, or all, chemical and biological activities or properties of a wild-type or mutant LXR polypeptide. The term “modulation” as used herein refers to both upregulation (i.e., activation or stimulation) and downregulation (i.e. inhibition or suppression) of a response.

As used herein, the terms “LXR gene” and “recombinant LXR gene” mean a nucleic acid molecule comprising an open reading frame encoding a LXR polypeptide of the present invention, including both exon and (optionally) intron sequences.

As used herein, the term “gene” is used for simplicity to refer to a functional protein, polypeptide or peptide encoding unit. As will be understood by those in the art, this functional term includes both genomic sequences and cDNA sequences. Preferred embodiments of genomic and cDNA sequences are disclosed herein.

As used herein, the term “DNA sequence encoding a LXR polypeptide” can refer to one or more coding sequences within a particular individual. Moreover, certain differences in nucleotide sequences can exist between individual organisms, which are called alleles. It is possible that such allelic differences might or might not result in differences in amino acid sequence of the encoded polypeptide yet still encode a protein with the same biological activity. As is well known, genes for a particular polypeptide can exist in single or multiple copies within the genome of an individual. Such duplicate genes can be identical or can have certain modifications, including nucleotide substitutions, additions or deletions, all of which still code for polypeptides having substantially the same activity.

As used herein, the term “intron” means a DNA sequence present in a given gene that is not translated into protein.

As used herein, the term “interact” means detectable interactions between molecules, such as can be detected using, for example, a yeast two hybrid assay. The term “interact” is also meant to include “binding” interactions between molecules. Interactions can, for example, be protein-protein or protein-nucleic acid in nature.

As used herein, the terms “cells,” “host cells” or “recombinant host cells” are used interchangeably and mean not only to the particular subject cell, but also to the progeny or potential progeny of such a cell. Because certain modifications can occur in succeeding generations due to either mutation or environmental influences, such progeny might not, in fact, be identical to the parent cell, but are still included within the scope of the term as used herein.

As used herein, the term “agonist” means an agent that supplements or potentiates the bioactivity of a functional LXR gene or protein, of a polypeptide encoded by a gene that is up- or down-regulated by a LXR polypeptide, and/or a polypeptide encoded by a gene that contains a LXR binding site in its promoter region.

As used herein, the term “antagonist” means an agent that decreases or inhibits the bioactivity of a functional LXR gene or protein, or that supplements or potentiates the bioactivity of a naturally occurring or engineered non-functional LXR gene or protein. Alternatively, an antagonist can decrease or inhibit the bioactivity of a functional gene or polypeptide encoded by a gene that is up- or down-regulated by a LXR polypeptide and/or contains a LXR binding site in its promoter region. An antagonist can also supplement or potentiate the bioactivity of a naturally occurring or engineered non-functional gene or polypeptide encoded by a gene that is up- or down-regulated by a LXR polypeptide, and/or contains a LXR binding site in its promoter region.

As used herein, the terms “chimeric protein” or “fusion protein” are used interchangeably and mean a fusion of a first amino acid sequence encoding a LXR polypeptide with a second amino acid sequence defining a polypeptide domain foreign to, and not homologous with, any domain of one of a LXR polypeptide. A chimeric protein can present a foreign domain that is found in an organism that also expresses the first protein, or it can be an “interspecies” or “intergenic” fusion of protein structures expressed by different kinds of organisms. In general, a fusion protein can be represented by the general formula X—LXR—Y, wherein LXR represents a portion of the protein which is derived from a LXR polypeptide, and X and Y are independently absent or represent amino acid sequences which are not related to a LXR sequence in an organism, which includes naturally occurring mutants. The term “chimeric gene” refers to a nucleic acid construct that encodes a “chimeric protein” or “fusion protein” as defined herein.

As used herein, the term “therapeutic agent” is a chemical entity intended to effectuate a change in an organism. Preferably, but not necessarily, the organism is a human being. It is not necessary that a therapeutic agent be known to effectuate a change in an organism; chemical entities that are suspected, predicted or designed to effectuate a change in an organism are therefore encompassed by the term “therapeutic agent.” The effectuated change can be of any kind, observable or unobservable, and can include, for example, a change in the biological activity of a protein.

Representative therapeutic compounds include small molecules, proteins and peptides, oligonucleotides of any length, “xenobiotics”, such as drugs and other therapeutic agents, carcinogens and environmental pollutants, natural products and extracts, as well as “endobiotics”, such as epoxycholesterols. Other examples of therapeutic agents can include, but are not restricted to, agonists and antagonists of a LXR polypeptide, toxins and venoms, viral epitopes, hormones (e.g., opioid peptides, steroids, etc.), hormone receptors, peptides, enzymes, enzyme substrates, co-factors, lectins, sugars, oligonucleotides or nucleic acids, oligosaccharides, proteins, small molecules and monoclonal antibodies.

II. Description of Tables

Table 1 is a table summarizing the crystal and data statistics obtained from the crystallized ligand binding domain of human LXR. Data on the unit cell are presented, including data on the crystal space group, unit cell dimensions, molecules per asymmetric cell and crystal resolution.

Table 2 is a table presenting the atomic coordinate data for crystallized LXRβ LBD in complex with EPC and a SRC1 peptide.

Table 3 is a table presenting the atomic coordinate data for crystallized LXRβ LBD in complex with N-(2,2,2-trifluoroethyl)-N-[4-(2,2,2-trifluoro-1-hydroxy-1-trifluoromethyl-ethyl)-phenyl]-benzenesulfonamide.

Table 4 is a table presenting the atomic coordinate data for human RARγ employed in the molecular replacement solution of human LXRβ ligand binding domain crystals.

III. General Considerations

The present invention will usually be applicable mutatis mutandis to all LXR polypeptides, as discussed herein, based, in part, on the patterns of LXR structure and modulation that have emerged as a consequence of determining a preferred three dimensional structure, human LXR in complex with a ligand. Generally speaking, LXR homologs and orthologs display substantial regions of amino acid homology. Additionally, the LXRs display an overall structural motif comprising three modular domains:

1) a variable amino-terminal domain;

2) a highly conserved DNA-binding domain (DBD); and

3) a less conserved carboxy-terminal ligand binding domain (LBD).

The modularity of LXR permits different domains of each protein to separately accomplish different functions, although the domains can influence each other. The separate function of a domain is usually preserved when a particular domain is isolated from the remainder of the protein. Using conventional protein chemistry techniques, a modular domain can sometimes be separated from the parent protein. Using conventional molecular biology techniques, each domain can usually be separately expressed with its original function intact or, as discussed herein below, chimeric proteins comprising two different proteins can be constructed, wherein the chimeric proteins retain the properties of the individual functional domains of the respective polypeptides from which the chimeric proteins were generated.

The amino terminal domain of LXR is the least conserved of the three domains. This domain is involved in transcriptional activation and, in some cases, its uniqueness can dictate selective receptor-DNA binding and activation of target genes by LXR.

The DBD is the most conserved structure in LXR. It typically contains about 70 amino acids that fold into two zinc finger motifs, wherein a zinc ion coordinates four cysteines. The DBD generally contains two perpendicularly oriented α-helices that extend from the base of the first and second zinc fingers. The two zinc fingers function in concert along with non-zinc finger residues to direct the LXRs to specific target sites on DNA. Various amino acids in the DBD influence spacing between two half-sites (which usually comprises six nucleotides) for receptor homodimerization. The optimal spacings facilitate cooperative interactions between DBDs, and D box residues are part of the dimerization interface. Other regions of the DBD facilitate DNA-protein and protein-protein interactions required for LXR homodimerization.

The LBD is the second most highly conserved domain in these receptors. Whereas the integrity of several different LBD sub-domains is important for ligand binding, truncated molecules containing only the LBD can retain normal ligand binding activity. This domain also participates in other functions, including dimerization, nuclear translocation and transcriptional regulation activities. Importantly, this domain can bind a ligand and can undergo ligand-induced conformational changes. Ligand binding allows the activation domain to serve as an interaction site for essential co-activator proteins that function to stimulate or inhibit transcription.

The carboxy-terminal activation subdomain is in close three-dimensional proximity in the LBD to the ligand, so as to allow for ligands bound to the LBD to coordinate (or interact) with amino acid(s) in the activation subdomain. As disclosed herein, the LBD of LXR is expressed, crystallized and its three dimensional structure determined. Computational and other methods for the design of ligands to the LBD are also disclosed.

IV. Production of LXR Polypeptides

The native and mutated LXR polypeptides, and fragments thereof, of the present invention can be chemically synthesized in whole or part using techniques that are well-known in the art (See, e.g., Creighton, (1983) Proteins: Structures and Molecular Principles, W. H. Freeman & Co., New York, incorporated herein in its entirety). Alternatively, methods that are well known to those skilled in the art can be used to construct expression vectors containing a partial or the entire native or mutated LXR polypeptide coding sequence and appropriate transcriptional/translational control signals. These methods include in vitro recombinant DNA techniques, synthetic techniques and in vivo recombination/genetic recombination. See, for example, the techniques described in Sambrook et al., (1989) Molecular Cloning: A Laboratory Manual, Cold Spring Harbor Laboratory, New York, and Ausubel et al., (1989) Current Protocols in Molecular Biology, Greene Publishing Associates and Wiley Interscience, New York, both incorporated herein in their entirety.

A variety of host-expression vector systems can be utilized to express a LXR coding sequence. These include but are not limited to microorganisms such as bacteria transformed with recombinant bacteriophage DNA, plasmid DNA or cosmid DNA expression vectors containing a LXR coding sequence; yeast transformed with recombinant yeast expression vectors containing a LXR coding sequence; insect cell systems infected with recombinant virus expression vectors (e.g., baculovirus) containing a LXR coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing a LXR coding sequence; or animal cell systems. The expression elements of these systems vary in their strength and specificities.

Depending on the host/vector system utilized, any of a number of suitable transcription and translation elements, including constitutive and inducible promoters, can be used in the expression vector. For example, when cloning in bacterial systems, inducible promoters such as pL of bacteriophage λ, plac, ptrp, ptac (ptrp-lac hybrid promoter) and the like can be used. When cloning in insect cell systems, promoters such as the baculovirus polyhedrin promoter can be used. When cloning in plant cell systems, promoters derived from the genome of plant cells, such as heat shock promoters; the promoter for the small subunit of RUBISCO; the promoter for the chlorophyll a/b binding protein) or from plant viruses (e.g., the 35S RNA promoter of CaMV; the coat protein promoter of TMV) can be used. When cloning in mammalian cell systems, promoters derived from the genome of mammalian cells (e.g., metallothionein promoter) or from mammalian viruses (e.g., the adenovirus late promoter; the vaccinia virus 7.5K promoter) can be employed. When generating cell lines that contain multiple copies of the tyrosine kinase domain DNA, SV40-, BPV- and EBV-based vectors can be used with an appropriate selectable marker.

V. Formation of LXR Ligand Binding Domain Crystals

In one embodiment, the present invention provides crystals of LXR. The crystals were obtained using the methodology disclosed in the Laboratory Examples. The LXR crystals, which can be native crystals, derivative crystals or co-crystals, have orthorhombic unit cells (an orthorhombic unit cell is a unit cell wherein a≠b≠c, and wherein α=β=γ90°) and space group symmetry of P2₁2₁2₁ in one embodiment and C222₁ in another embodiment. There are two LXR molecules in the asymmetric unit related by a non-crystallographic dyad. In one LXR crystalline form, the unit cell has dimensions of a=60.25 Å, b=82.454 Å, c=123.175 Å, and α=β=γ=90°. In another LXR crystalline form, the unit cell has dimensions of a=71.166 A, b=120.012 Å, c=147.56 Å, and α=β=γ=90°.

The structure of the N-(2,2,2-trifluoroethyl)-N-[4-(2,2,2-trifluoro-1-hydroxy-1-trifluoromethyl-ethyl)-phenyl]-benzenesulfonamide-bound form of LXR was solved by molecular replacement using the structure of the human retinoic acid receptor γ (RARγ) as a search model (Table 4) (Renaud et al., (1995) Nature 378: 681-689; PDB ID No; 2LBD, coordinates available online at http://www.rcsb.org/pd b/). The N-(2,2,2-trifluoroethyl)-N-[4-(2,2,2-trifluoro-1-hydroxy-1-trifluoromethyl-ethyl)-phenyl ]-benzenesulfonamide-bound form was refined to a resolution of about 2.3 Å. The structure of the EPC-bound form of LXR was solved by molecular replacement using the refined LXR subunit from the N-(2,2,2-trifluoroethyl)-N-[4-(2,2,2-trifluoro-1-hydroxy-1-trifluoromethyl-ethyl)-phenyl]-benzenesulfonamide-bound form as a search model. The EPC-bound form was refined to a resolution of about 2.8 Å.

The heavy atom derivatized form of the LXR LBD-ligand structure can be solved using single isomorphous replacement anomalous scattering (SIRAS) techniques and/or multiwavelength anomalous diffraction (MAD) techniques. In the SIRAS method of solving protein crystals, a derivative crystal is prepared that contains an atom that is heavier than the other atoms of the sample. Heavy atom derivative crystals are commonly prepared by soaking a crystal in a solution containing a selected heavy atom salt. For example, some heavy atom derivative crystals have been prepared by soaking a crystalline form of the protein of interest in a solution of methyl mercury chloride (MeHgCl). Another representative heavy atom that can be incorporated into a derivative crystal is iodine. Heavy atoms can associate with the protein of interest, or can be localized in a ligand that associates with a protein of interest.

Analysis of derivative crystals takes advantage of differences in the reflections from the derivative crystal as compared to the underivatized crystal. Symmetry-related reflections in the X-ray diffraction pattern, which are usually identical, are altered by the anomalous scattering contribution of the heavy atoms. The measured differences in symmetry-related reflections are used to determine the position of the heavy atoms, leading to an initial estimation of the diffraction phases, and subsequently, an electron density map is prepared. The prepared electron density map is then used to identify the position of the other atoms in the sample.

V.A. Preparation of LXR Crystals

The native and derivative co-crystals, and fragments thereof, disclosed in the present invention can be obtained by a variety of techniques, including batch, liquid bridge, dialysis, vapor diffusion and hanging drop methods (See, e.g., McPherson, (1982) Preparation and Analysis of Protein Crystals, John Wiley, New York; McPherson, (1990) Eur. J. Biochem. 189:1-23; Weber, (1991) Adv. Protein Chem. 41:1-36). In a preferred embodiment, the vapor diffusion and hanging drop methods are used for the crystallization of LXR polypeptides and fragments thereof.

In general, native crystals of the present invention are grown by dissolving substantially pure LXR LBD polypeptide or a fragment thereof in an aqueous buffer containing a precipitant at a concentration just below that necessary to precipitate the protein. Water is removed by controlled evaporation to produce precipitating conditions, which are maintained until crystal growth ceases.

In a preferred embodiment of the invention, native crystals are grown by the hanging drop method (See, e.g., McPherson, (1982) Preparation and Analysis of Protein Crystals, John Wiley, New York; McPherson, (1990) Eur. J. Biochem. 189:1-23). In this method, the polypeptide/precipitant solution is allowed to equilibrate in a closed container with a larger aqueous reservoir having a precipitant concentration optimal for producing crystals. Generally, less than about 25 μL of LXR LBD polypeptide solution is mixed with an equal volume of reservoir solution, giving a precipitant concentration about half that required for crystallization. This solution is suspended as a droplet underneath a coverslip, which is sealed onto the top of the reservoir. The sealed container is allowed to stand, until crystals grow. Crystals generally form within two to six weeks, and are suitable for data collection within approximately seven to ten weeks. Of course, those of skill in the art will recognize that the above-described crystallization procedures and conditions can be varied.

V.B. Preparation of Derivative Crystals

Derivative crystals of the present invention, e.g. heavy atom derivative crystals, can be obtained by soaking native crystals in mother liquor containing salts of heavy metal atoms. Alternatively, a ligand comprising a heavy atom can be associated with a protein, and subsequently co-crystallized. Such derivative crystals are useful for phase analysis in the solution of crystals of the present invention. This mechanism provides derivative crystals suitable for use as isomorphous replacements in determining the X-ray crystal structure of a LXR polypeptide. Additional reagents useful for the preparation of the derivative crystals of the present invention will be apparent to those of skill in the art after review of the disclosure of the present invention presented herein.

V.C. Preparation of Co-Crystals

Co-crystals of the present invention can be obtained by soaking a native crystal in mother liquor containing compounds known or predicted to bind the LBD of a LXR, or a fragment thereof. Alternatively, co-crystals can be obtained by co-crystallizing a LXR LBD polypeptide or a fragment thereof in the presence of one or more compounds known or predicted to bind the polypeptide. In a preferred embodiment of the present invention, for example, the ligand N-(2,2,2-trifluoroethyl)-N-[4-(2,2,2-trifluoro-1-hydroxy-1-trifluoromethyl-ethyl)-phenyl]-benzenesulfonamide is co-crystallized with LXR. In another embodiment, an epoxycholesterol is co-crystallyzed with LXR and a peptide derived from a steroid receptor coactivator (SRC).

V.D. Solving a Crystal Structure of the Present Invention

Crystal structures of the present invention can be solved using a variety of techniques including, but not limited to, isomorphous replacement anomalous scattering or molecular replacement methods. Computer software packages will also be helpful in solving a crystal structure of the present invention. Applicable software packages include but are not limited to AmoRe (Navaza & Saludjian, (1997) Method Enzymol. 276A: 581-94; AmoRe is available on the internet at various sites, such as ftp:/ftp.informatik.uni-kiel.de/pub/kiel/amore), X-PLOR™ program (Brünger, (1992) X-PLOR, Version 3.1. A System for X-ray Crystallography and NMR, Yale University Press, New Haven, Conn.; X-PLOR is available from Molecular Simulations, Inc., San Diego, Calif.), Xtal View (McRee, (1992) J. Mol. Graphics 10: 44-47; X-tal View is available from the San Diego Supercomputer Center), SHELXS 97 (Sheldrick (1990) Acta Cryst. A 46: 467; SHELX 97 is available from the Institute of Inorganic Chemistry, Georg-August-Universität, Göttingen, Germany), HEAVY (Terwilliger, Los Alamos National Laboratory) can be used and SHAKE-AND-BAKE (Hauptman, (1997) Curr. Opin. Struct. Biol. 7: 672-80; Weeks et al., (1993) Acta Cryst. D 49: 179; available from the Hauptman-Woodward Medical Research Institute, Buffalo, N.Y.). See also, Ducruix & Geige, (1992) Crystallization of Nucleic Acids and Proteins: A Practical Approach, IRL Press, Oxford, England, and references cited therein.

VI. Summary of Results for the LXR Ligand binding Domain

Three structures of the LBD of human LXR are disclosed in the present invention. First, a 2.3 Å stucture in complex with the ligand N-(2,2,2-trifluoroethyl)-N-[4-(2,2,2-trifluoro-1-hydroxy-1-trifluoromethyl-ethyl)-phenyl]-benzenesulfonamide is disclosed, second, a 2.8 Å structure in complex with an epoxycholesterol and a SRC pepide is disclosed.

The crystal structures of the present invention disclose important insights into how human LXR binds ligands and thus, can be useful in designing LXR ligands, including activators and inhibitors. These structures reveal that although LXR has a well defined hydrophobic pocket, the binding modes of different compounds can differ radically. A hydrogen bond with histidine 435 was found to be the only conserved polar interaction. Also, the structures revealed that filling the volume of the pocket was not necessary nor sufficient for activation of LXR.

VI.A. Overall Structure of the Human LXR Ligand Binding Domain

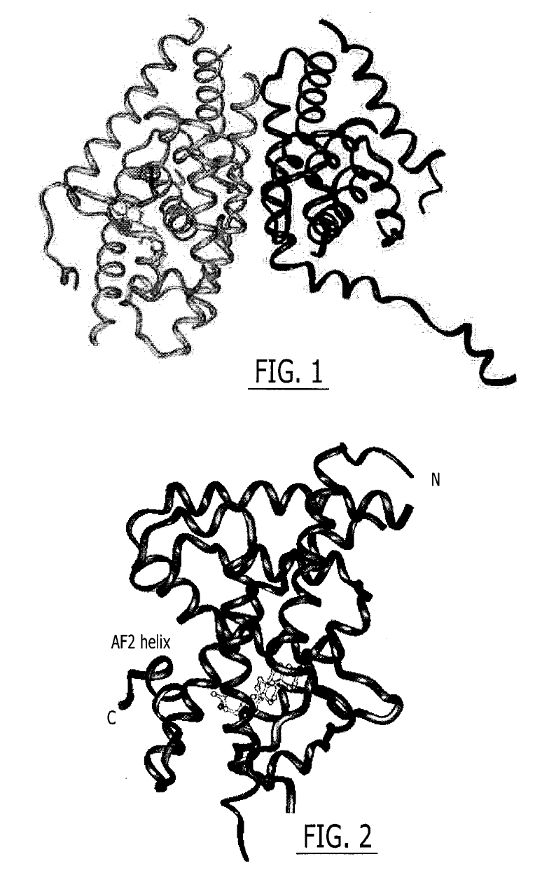

The LXRβ LBD had the same three-layered α-helical fold seen for other nuclear receptors (see FIG. 1). In all complex structures, the asymmetric unit contains a LXRβ homodimer, with the T317 complex and the EPC/SRC1(2) complex containing two holo molecules. Although the non-crystallographic molecules are identical sequentially, the marked structural differences observed are most likely due to crystal packing forces. The dimer interface is composed of residues in helices 7, 9, 10 and 11, as seen in other homo and heterodimer interfaces. Each homodimer comprises A and B subunits.

To understand the degree of flexibility in the LXRβ LBD, the deviation in Cα atoms in the LBD core region, comprising helices 3 to 11 was compared. The two most similar molecules were those binding EPC with the SRC peptide, having an average deviation of Cα atoms of 0.43 A RMSD. The two molecules binding the N-(2,2,2-trifluoroethyl)-N-[4-(2,2,2-trifluoro-1-hydroxy-1-trifluoromethyl-ethyl)-phenyl]-benzenesulfonamide compound, with an average deviation of Cα atoms of 0.6 A RMSD. Those molecules binding EPC differ from those binding the N-(2,2,2-trifluoroethyl)-N-[4-(2,2,2-trifluoro-1-hydroxy-1-trifluoromethyl-ethyl)-phenyl]-benzenesulfonamide compound by an average deviation of 0.65 A RMSD. In all cases, the Cα positional variance between structures increased proportionally with the distance of the residue from the homodimer interface.

The most distinguishing features of the canonical LXRβ structure are a large helix 1 (˜18 residues) and a relatively large pocket (833 Å³) compared with previously solved steroid receptors. This large binding pocket is consistent with the larger size of oxy-cholesterol derivatives compared to the sex steroids.

VI.B. The LXRβ/epoxycholesterol Complex

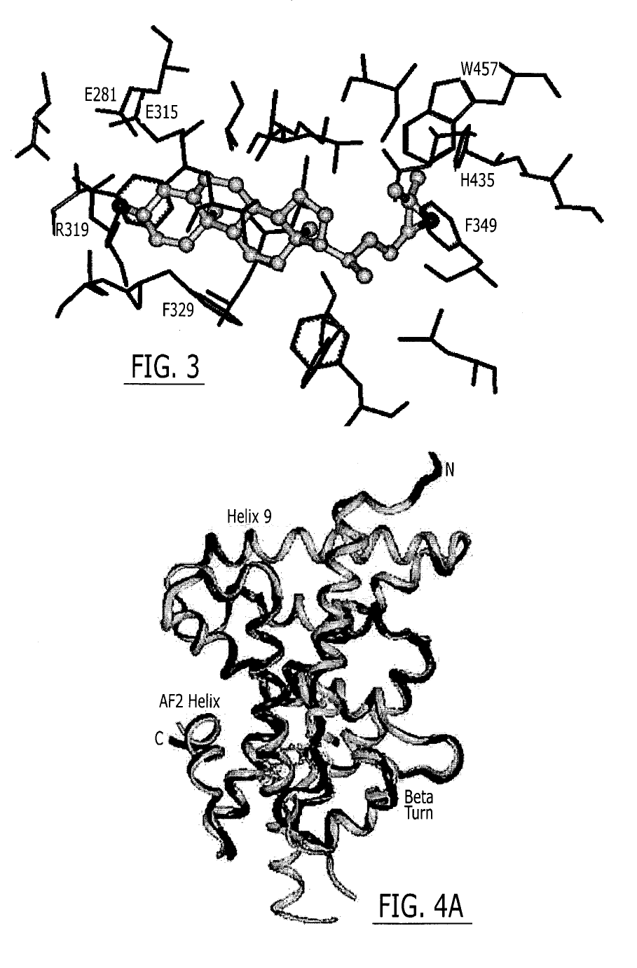

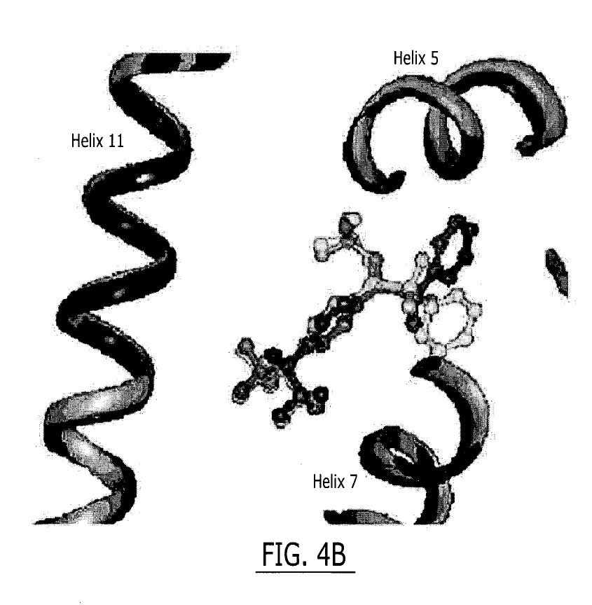

LXRβ with bound EPC crystallized as a dimer (see FIG. 1), with the same dimer interface seen with other nuclear receptors with EPC filling most of the pocket. The binding of EPC to LXRβ differs significantly from bound steroids observed in previously solved nuclear receptors (Brzozowski et al., (1997) Nature 389:753-758; Williams & Sigler, (1998) Nature 393:392-396; Matias et al., (2000) J. Biol. Chem. 275:26164-2617). Like other steroid receptors, EPC bound to LXRβ with the steroid A-ring oriented towards helices 3 and 4, and the D-ring and tail pointing towards helix 11 and the AF2. Although the steroid cores of progesterone, estradiol, and testosterone overlap almost perfectly when the corresponding nuclear receptor's Cα atoms are superimposed, the steroid core of EPC did not overlap that of the sex steroids when the Cα atoms of LXRβ were superimposed on those receptors. Additionally, the steroid core of EPC is flipped 180° around its long axis, so that the EPC angular methyls pointed in the opposite direction from those of other steroids, like progesterone (see FIG. 2).

The binding site for EPC was almost exclusively hydrophobic, with the only hydrophilic interaction at the end of the pocket near the A-ring ketone oxygen (O3) where LXR and the EPC O3 form a network of interactions. The O3 of EPC is positioned in a polar pocket containing Glu281, Glu315, and Arg319. The side chains of Phe 243 and Phe329 packed against the hydrophobic A ring of EPC, and pushed it against the Cp and Cy of Glu315. The steric interaction between EPC and Glu315 positioned the acid group to form a salt bridge with Arg319. This salt bridge gave Arg319 the proper orientation to form the hydrogen bond with O3 of EPC (2.6 Å). The orientation of the Glu315 acid head group was fixed by a hydrogen bond with Arg318 which was fixed to Arg319 by the peptide bond.

There is a steric interaction between the side chain of Trp457 and the EPC 24(S), 25 epoxide group. The epoxide oxygen is ˜3.2 Å from two carbons in the six-member ring of the Trp457 indole. The Trp457 indole is oriented such that the nitrogen atom is pointed away from EPC, making it impossible to form a direct hydrogen bond with the epoxide oxygen (Spencer et al., (2001) J. Med. Chem. 44: 886-897). However, the EPC epoxide oxygen is positioned to form a weak hydrogen bond with His435 (3.4 Å), explaining why a correctly positioned oxygen atom is required distal to EPC C22 to activate LXR. The interaction with the epoxide oxygen orients the His435 imidazole basic nitrogen away from the Trp457 indole, providing a hydrophobic surface for the side chain top pack against. It is possible that this indirect interaction is responsible for the Trp457 requirement observed previously, a smaller side chain cannot pack against His435. The position of the AF2 helix was stabilized by the presence of SRC located in the co-activator grove.

All residues in LXRβ lining the binding pocket that are within 5 Å of the epoxycholesterol molecule include: Asn239 and Phe243 in helix 1; Phe 268, Phe271, Thr272, Leu274, Ala275, Ser278, and Glu281 in helix 3; Ile309, Met312, Leu313, Glu315, Thr316 and Arg319 in helix4/5; Phe329 in the beta turn; Phe340 in helix 6; Leu345, Phe349, and Ile353 in helix 7; His435, Gln438, Val439, and Leu442 in helix 11, Leu449, Leu453, and Trp457 in the AF2 helix. There were several areas of unoccupied space in the binding pocket adjacent to the EPC C and D rings, proximal to Met312 and Thr316, and proximal to Ile327 and Phe340.

VI.C. The LXRβ/T317 Complex

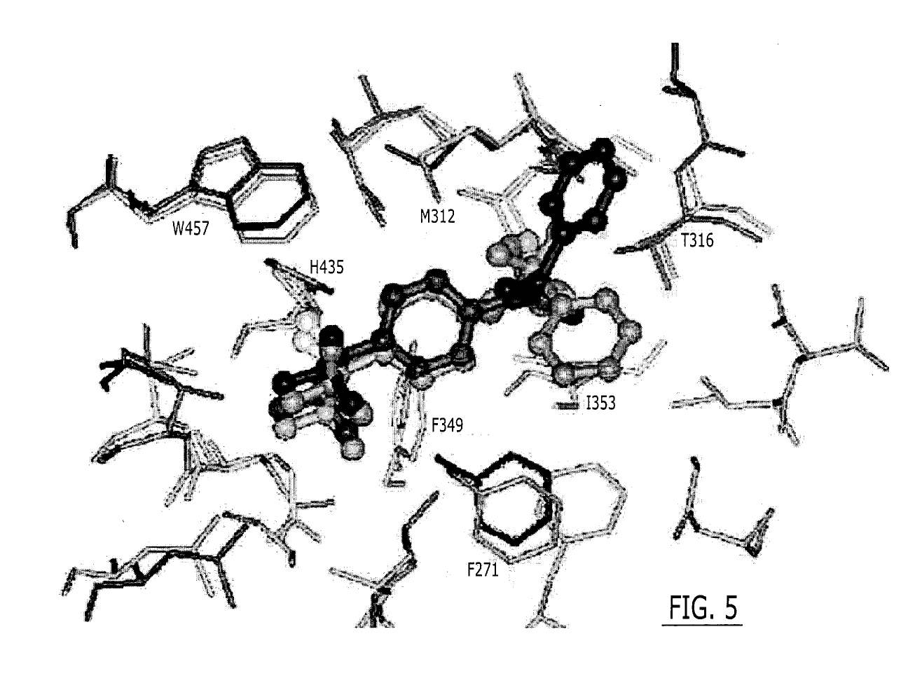

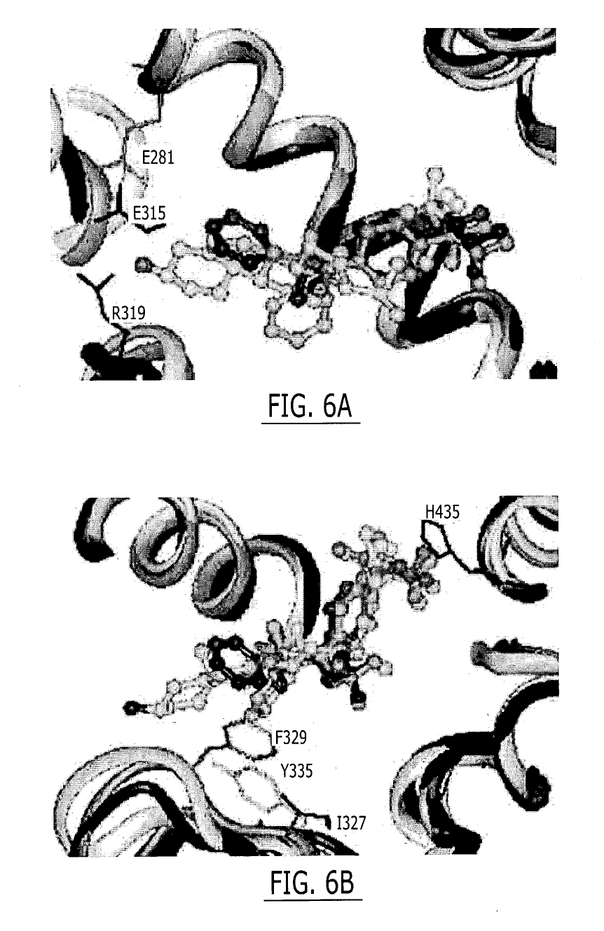

The structure of the complex of LXRβ with compound T317 yielded two distinct LXRβ conformations, with T317 adopting both syn and gauche conformations within the relatively large binding pocket (FIGS. 4 and 5). These multiple conformations were likely present because of a combination of crystal packing forces and a low rotational energy barrier around the compound's S—N bond. When corresponding Cα atoms were superimposed, both LXRβ molecules had nearly identical conformations surrounding the hexafluoroisopropyl moiety. This part of the receptor, which comprises helices 7 through 11, also displayed the greatest degree of conservation between the two molecules in the EPC structure, presumably because the homodimer interface forced a local dyad symmetry. The only polar interaction observed was a hydrogen bond between the hexafluoro-isopropyl oxygen atom and the His435 imidazole nitrogen. This interaction was present in both copies of the molecule and is probably responsible for the tight binding and activation by this compound.

Interacting residues whose side chain conformations are conserved between the two molecules include Phe271, Thr272, Ile309, Leu345, Phe349, His435, Gln438, Val439, Leu442, Leu449, Leu453, and Trp457. Other residues that interact with both conformations of T317 include Leu274, Ala275, Ser278, Met312, Leu313, Thr316, and Ile353. Residues that interact with the gauche conformation (A molecule) but not the syn are in the beta-turn region and include Ile327, Thr328, and Phe329. This conformation of T317 fills the pocket seen in the LXRβ/EPC complex. Residues that interact with the syn conformation (B molecule) but not the gauche include residues in helices 3 and 4 that interact with the EPC A-ring, and include Phe268, Glu281, Glu315, Tyr335, and Phe340. Neither copy of T317 filled the LXRb pocket, both left significant voids in different regions of the pocket.

VI.D. Mutational Analysis of LXR Ligand Interactions

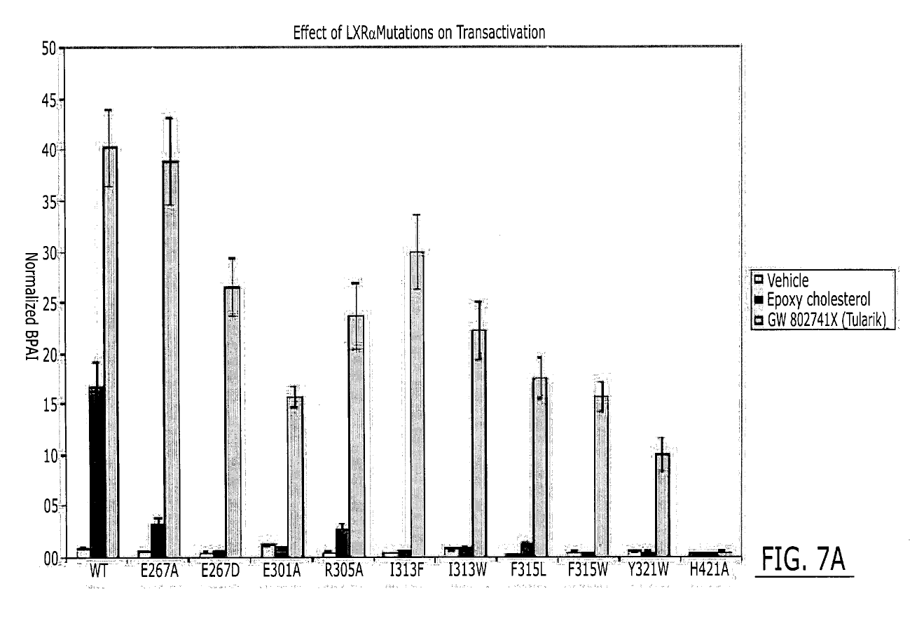

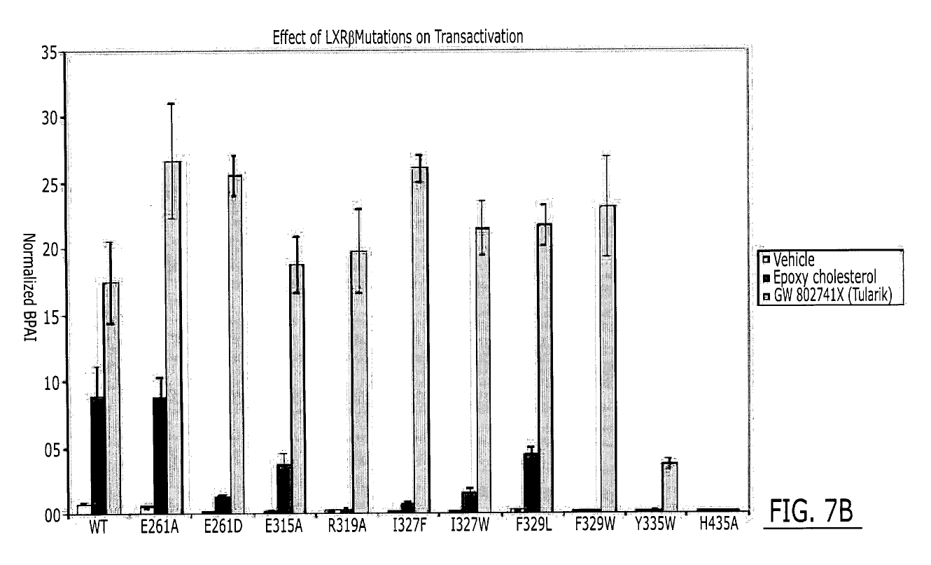

The structures of the complex of LXRβ with bound T317 and EPC revealed strikingly different protein/ligand interactions. To explore these differences, site directed mutants were made targeting specific pocket residues that interacted more directly with one compound than the other. FIG. 6A indicates that both T317 and EPC hydrogen bond with H435 (H421α). However, the gauche conformation of I317 extended into a pocket near the beta turn. This observation suggests that mutations of I327 (I313α), F329 (F315α), and Y335 (Y321α) that close off the pocket might be used to test whether this region is significant for compound binding. By contrast, O3 of EPC extended into a hydrophilic pocket (see FIG. 6B) which includes residues E281 (E267α), E315 (E301α), and R319 (R305α). Mutations in this region permit identification of the necessary polar interactions for EPC binding. The transactivation capability of gal4-fusions of wild-type and mutant LXRα and LXRβ LBDs were measured by their ability to transcribe secreted placental alkaline phosphatase (SPAP) on a UAS-tk promoter in transient transfection experiments in CV-1 cells. See Laboratory Example 4. FIGS. 7A and 7B show the effects of these mutations on the transcriptional activity of LXR. As expected, only the H435A (H421Acc) mutation completely knocked out the activity of T317, with some modulation by the Y335W mutation. Although most mutations decreased EPC activity, removing R319 (R306α) and H435 (H421α) completely abolished LXR activation, in accordance with the structure. These data agree with the structure in suggesting that the conserved arginine R319 (R305α) is the critical residue for organizing the polar interactions around EPC O3. Also in accordance with the structure, adding bulk to F329 (F329W) sterically inhibited EPC binding while decreasing bulk (F329L) did not. Although the mutational data from both LXR subtypes is similar, mutations in LXRα appear to have a greater impact than mutations in LXRβ, suggesting that the pocket is tighter.

VI.E. The Pharmacology of Oxysterols

From previous structure activity relationship studies performed on oxysterol activation of LXRα, Trp443 was found to be essential for activation. This residue is equivalent to to the Trp457 residue of LXRβ (Spencer et al., (2001) J. Med. Chem. 44:886-897). These studies also concluded that a hydrogen-bonding group on the oxysterol C24 was essential for activation. The structure of LXRβ in complex with EPC makes it clear why the tryptophan is essential, and why the epoxide group is required for activation. The conserved hydrogen bond with His435 seen in both complexes underscores its importance for activation. Only oxysterols able to make this hydrogen bond can orient H435 to form a hydrophobic complementary surface for W457, allowing the AF2 helix to pack appropriately. Smaller residues than a Trp will not interact with H435, so the hydrogen bond between the histidine and the oxysterol will not be strong enough. The structure also suggests that oxysterols without the hydrogen bonding group, like cholesterol, should bind to LXR, but will not activate transcription through the AF2. Newer binding data suggests that cholesterol and other oxysterols do bind to LXR but do not recruit co-activator, an observation that would not detected in those previous studies because it was not looked for. This property of the oxysterols is especially relevant to drug discovery efforts, since many compounds also bind tightly but do not activate LXR.

VI.F. Essential Contacts for Ligand Binding

In all four holo molecules, the ligand made contacts with residues in helices 3, 4, 5, the beta-turn, helix 11, and the AF2 helix, and was oriented in the same pocket observed for other nuclear receptors. In general, the protein ligand interface was dominated by hydrophobic contacts. The interaction with His435 is conserved in all three liganded molecules, suggesting that interactions with this residue is necessary for LXR activation, but could be different for the two different classes of ligand. This result agrees with earlier mutagenesis data, which showed that LXRα Trp443 (Trp457β) was essential for activation by oxysterols, but not by T317. The other polar residue essential for oxysterol binding was R319, with F329 required to position the oxysterol A-ring. However, in light of the tight and efficacious nature of T317, interaction with these residues is neither necessary nor sufficient for LXR activation.

Another interesting feature of the LXR/T317 complex is that the ligand does not fill the pocket. Indeed, there is enough room in the pocket for T317 to adopt multiple conformations, yet the compound bound tightly and was efficacious. Thus, although it is not applicants' desire to be bound by any theory of operation, the structures of T317 and EPC, combined with the binding data on oxysterols, imply that filling the LXR binding pocket is neither necessary nor sufficient for activation.

VI.G. Generation of Easily-Solved LXR Crystals

The present invention discloses a substantially pure LXR LBD polypeptide in crystalline form. In preferred embodiments, exemplified in the Figures and Laboratory Examples, LXR is crystallized with bound ligand(s). Crystals are formed from LXR LBD polypeptides that are usually expressed by a cell culture, such as E. coli. Bromo-, iodo- and substitutions can be included during the preparation of crystal forms and can act as heavy atom substitutions in LXR ligands and in crystals of LXR and the LXR LBD. This method can be advantageous for the phasing of the crystal, which is a crucial, and sometimes limiting, step in solving the three-dimensional structure of a crystallized entity. Thus, the need for generating the heavy metal derivatives traditionally employed in crystallography might be eliminated. After the three-dimensional structure of a LXR or LXR LBD with or without a ligand bound is determined, the resultant three-dimensional structure can be used in computational methods to design synthetic ligands for LXR and other LXR polypeptide fragments. Further activity structure relationships can be determined through routine testing, using assays disclosed herein and known in the art.

VII. Preparation of LXR Ligands

The ligand N-(2,2,2-trifluoroethyl)-N-[4-(2,2,2-trifluoro-1-hydroxy-1-trifluoromethyl-ethyl)-phenyl]-benzenesulfonamide (T317) has been previously described (Schultz et al., (2000) Gene Dev. 14: 2831-2838) and comprises the chemical structure:

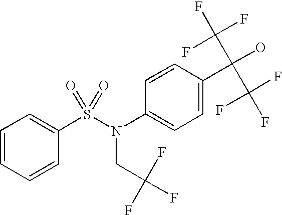

The ligand 24(S), 25-epoxycholesterol (EPC) is commercially available and its utility as a ligand for LXR has been described (Lehmann, et al., (1997) J. Biol. Chem. 272(6): 3137-3140). The synthesis of this compound has also been described (Nelson et al., (1981) J. Biol. Chem. 256:1067-1068; Saucier et al., (1985) J. Biol. Chem. 260: 14571-14579). 24(S), 25-epoxycholesterol comprises the chemical structure:

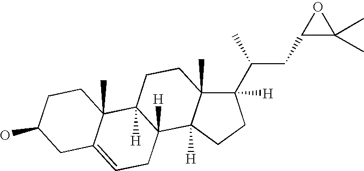

VIII. Uses of LXR Crystals and the Three-Dimensional Structure of the Ligand Binding Domain of LXR

The LXR crystals and three-dimensional structures that form aspects of the present invention can be employed in a variety of applications. A brief discussion of several of these applications is presented hereinbelow. Additional uses for LXR crystals and the three-dimensional structures disclosed herein will be apparent to those of skill in the art upon consideration of the present disclosure.

VIII.A. Design and Development of LXR Modulators

The knowledge of the structure of the LXR ligand binding domain, an aspect of the present invention, provides a tool for investigating the mechanism of action of LXR and other LXR polypeptides in a subject. For example, various computer models, as described herein, can predict the binding of various substrate molecules to the LBD of LXR. Upon discovering that such binding in fact takes place, knowledge of the protein structure then allows design and synthesis of small molecules that mimic the functional binding of the substrate to the LBD of LXR. This is the method of “rational” drug design, further described herein.

Use of the isolated and purified LXR crystalline structure of the present invention in rational drug design is thus provided in accordance with the present invention. Additional rational drug design techniques are described in U.S. Pat. Nos. 5,834,228 and 5,872,011, incorporated herein in their entirety.

Thus, in addition to the compounds described herein, other sterically similar compounds can be formulated to mimic the key structural regions of LXRs in general, or of hLXR in particular. The generation of a structural functional equivalent can be achieved by the techniques of modeling and chemical design known to those of skill in the art and described herein. It will be understood that all such sterically similar constructs fall within the scope of the present invention.

VIII.A.1. Rational Drug Design

The three-dimensional structure of a LXR ligand binding domain is unprecedented and will greatly aid in the development of new synthetic ligands for a LXR polypeptide, such as LXR agonists and antagonists, including those that bind exclusively to any one of the LXR orthologs. In addition, LXR is well suited to modern methods, including three-dimensional structure elucidation and combinatorial chemistry, such as those disclosed in U.S. Pat. No. 5,463,564, incorporated herein by reference. Structure determination using X-ray crystallography is possible because of the solubility properties of the LXR orthologs. Computer programs that use crystallography data when practicing the present invention will enable the rational design of ligands to these receptors. Programs such as RASMOL (Biomolecular Structures Group, Glaxo Wellcome Research & Development Stevenage, Hertfordshire, UK Version 2.6, August 1995, Version 2.6.4, December 1998, Copyright ©Roger Sayle 1992-1999) can be used with the atomic structural coordinates from crystals of the present invention, crystals generated by practicing the invention or crystals used to practice the invention by generating three-dimensional models and/or determining the structures involved in ligand binding. Computer programs such as those sold under the registered trademark INSIGHT II® and such as GRASP (Nicholls et al., (1991) Proteins 11: 281-96) allow for further manipulations and the ability to introduce new structures. In addition, high throughput binding and bioactivity assays can be devised using purified recombinant protein and modern reporter gene transcription assays known to those of skill in the art in order to refine the activity of a designed ligand.

A method of identifying modulators of the activity of a LXR polypeptide using rational drug design is thus provided in accordance with the present invention. The method comprises designing a potential modulator for a LXR polypeptide of the present invention that will form non-covalent bonds with amino acids in the ligand binding cavity based upon the crystalline structure of the LXR LBD polypeptide; synthesizing the modulator; and determining whether the potential modulator modulates the activity of the LXR polypeptide. In a preferred embodiment, the modulator is designed for a LXR polypeptide. Preferably, the LXR polypeptide comprises the amino acid sequence of SEQ ID NO: 4 and the LXR LBD comprises the amino acid sequence SEQ ID NO: 8. The determination of whether the modulator modulates the biological activity of a LXR polypeptide is made in accordance with the screening methods disclosed herein, or by other screening methods known to those of skill in the art. Modulators can be synthesized using techniques known to those of ordinary skill in the art.

In an alternative embodiment, a method of designing a modulator of a LXR polypeptide in accordance with the present invention is disclosed comprising: (a) selecting a candidate LXR ligand; (b) determining which amino acid or amino acids of a LXR polypeptide interact with the ligand using a three-dimensional model of a crystallized LXR LBD; (c) identifying in a biological assay for LXR activity a degree to which the ligand modulates the activity of the LXR polypeptide; (d) selecting a chemical modification of the ligand wherein the interaction between the amino acids of the LXR polypeptide and the ligand is predicted to be modulated by the chemical modification; (e) performing the chemical modification on the ligand to form a modified ligand; (f) contacting the modified ligand with the LXR polypeptide; (g) identifying in a biological assay for LXR activity a degree to which the modified ligand modulates the biological activity of the LXR polypeptide; and (h) comparing the biological activity of the LXR polypeptide in the presence of modified ligand with the biological activity of the LXR polypeptide in the presence of the unmodified ligand, whereby a modulator of a LXR polypeptide is designed.

VIII.A.2. Methods for Using the LXR LBD Structural Coordinates for Molecular Design

For the first time, the present invention permits the use of molecular design techniques to design, select and synthesize chemical entities and compounds, including modulatory compounds, capable of binding to the ligand binding cavity or an accessory binding site of LXR and the LXR LBD, in whole or in part. Correspondingly, the present invention also provides for the application of similar techniques in the design of modulators of any LXR polypeptide.

In accordance with a preferred embodiment of the present invention, the structure coordinates of a crystalline LXR LBD can be used to design compounds that bind to a LXR LBD (preferably a LXR LBD) and alter the properties of a LXR LBD (for example, the dimerization or ligand binding ability) in different ways. One aspect of the present invention provides for the design of compounds that act as competitive inhibitors of a LXR polypeptide by binding to all, or a portion of, the binding sites on a LXR LBD. The present invention also provides for the design of compounds that can act as uncompetitive inhibitors of a LXR LBD. These compounds can bind to all, or a portion of, an accessory binding site of a LXR that is already binding its ligand and can, therefore, be more potent and less non-specific than known competitive inhibitors that compete only for the LXR ligand binding cavity. Similarly, non-competitive inhibitors that bind to and inhibit LXR LBD activity, whether or not it is bound to another chemical entity, can be designed using the LXR LBD structure coordinates of this invention.

A second design approach is to probe a LXR or LXR LBD (preferably a hLXR or hLXR LBD) crystal with molecules comprising a variety of different chemical entities to determine optimal sites for interaction between candidate LXR or LXR LBD modulators and the polypeptide. For example, high resolution X-ray diffraction data collected from crystals saturated with solvent allows the determination of the site where each type of solvent molecule adheres. Small molecules that bind tightly to those sites can then be designed, synthesized and tested for their LXR modulator activity.

Once a computationally-designed ligand is synthesized using the methods of the present invention or other methods known to those of skill in the art, assays can be used to establish its efficacy of the ligand as a modulator of LXR (preferably hLXR) activity. After such assays, the ligands can be further refined by generating intact LXR, or LXR LBD, crystals with a ligand bound to the LXR. The structure of the ligand can then be further refined using the chemical modification methods described herein and known to those of skill in the art, in order to improve the modulation activity or the binding affinity of the ligand. This process can lead to second generation ligands with improved properties.

Ligands also can be selected that modulate LXR responsive gene transcription by the method of altering the interaction of co-activators and co-repressors with their cognate LXR. For example, agonistic ligands can be selected that block or dissociate a co-repressor from interacting with the LXR, and/or that promote binding or association of a co-activator. Antagonistic ligands can be selected that block co-activator interaction and/or promote co-repressor interaction with a target receptor. Selection can be done via binding assays that screen for designed ligands having the desired modulatory properties. Preferably, interactions of a hLXR polypeptide are targeted. Suitable assays for screening that can be employed, Mutatis mutandis in the present invention, are described in published PCT international applications WO 00/037077 and WO 00/025134, incorporated herein by reference in their entirety.

VIII.A.3. Methods of Designing LXR LBD Modulator Compounds

The design of candidate substances, also referred to as “compounds” or “candidate compounds”, that bind to or inhibit LXR LBD-mediated activity according to the present invention generally involves consideration of two factors. First, the compound must be capable of physically and structurally associating with a LXR LBD. Non-covalent molecular interactions important in the association of a LXR LBD with its substrate include hydrogen bonding, van der Waals interactions and hydrophobic interactions.

Second, the compound must be able to assume a conformation that allows it to associate with a LXR LBD. Although certain portions of the compound will not directly participate in this association with a LXR LBD, those portions can still influence the overall conformation of the molecule. This, in turn, can have a significant impact on potency. Such conformational requirements include the overall three-dimensional structure and orientation of the chemical entity or compound in relation to all or a portion of the binding site, e.g., the ligand binding cavity or an accessory binding site of a LXR LBD, or the spacing between functional groups of a compound comprising several chemical entities that directly interact with a LXR LBD.

The potential modulatory or binding effect of a chemical compound on a LXR LBD can be analyzed prior to its actual synthesis and testing by the use of computer modeling techniques that employ the coordinates of a crystalline LXR LBD polypeptide of the present invention. If the theoretical structure of the given compound suggests insufficient interaction and association between it and a LXR LBD, synthesis and testing of the compound is obviated. However, if computer modeling indicates a strong interaction, the molecule can then be synthesized and tested for its ability to bind and modulate the activity of a LXR LBD. In this manner, synthesis of unproductive or inoperative compounds can be avoided.

A modulatory or other binding compound of a LXR LBD polypeptide (preferably a hLXR LBD) can be computationally evaluated and designed via a series of steps in which chemical entities or fragments are screened and selected for their ability to associate with the individual binding sites or other areas of a crystalline LXR LBD polypeptide of the present invention.

One of several methods can be used to screen chemical entities or fragments for their ability to associate with a LXR LBD and, more particularly, with the individual binding sites of a LXR LBD, such as ligand binding cavity or an accessory binding site. This process can begin by visual inspection of, for example, the ligand binding cavity on a computer screen based on the LXR LBD atomic coordinates presented in Tables 2, 3 and 4. Selected fragments or chemical entities can then be positioned in a variety of orientations, or docked, within an individual binding site of a LXR LBD as defined herein above. Docking can be accomplished using software programs such as those available under the tradenames QUANTA™ (Molecular Simulations Inc., San Diego, Calif.) and SYBYL™ (Tripos, Inc., St. Louis, Mo.), followed by energy minimization and molecular dynamics with standard molecular mechanics forcefields, such as CHARM (Brooks et al., (1983) J. Comp. Chem., 8: 132) and AMBER 5 (Case et al., (1997) AMBER 5, University of California, San Francisco; Pearlman et al., (1995) Comput. Phys. Commun. 91: 1-41).

Specialized computer programs can also assist in the process of selecting fragments or chemical entities. These include:

1. GRID™ program, version 17 (Goodford, (1985) J. Med. Chem. 28: 849-57), which is available from Molecular Discovery Ltd., Oxford, UK;

2. MCSS™ program (Miranker & Karplus, (1991) Proteins 11: 29-34), which is available from Molecular Simulations, Inc., San Diego, Calif.;

3. AUTODOCK™ 3.0 program (Goodsell & Olsen, (1990) Proteins 8: 195-202), which is available from the Scripps Research Institute, La Jolla, Calif.;

4. DOCK™ 4.0 program (Kuntz et al., (1992) J. Mol. Biol. 161: 269-88), which is available from the University of California, San Francisco, Calif.;

5. FLEX-X™ program (See, Rarey et al., (1996) J. Comput. Aid. Mol. Des. 10:41-54), which is available from Tripos, Inc., St. Louis, Mo.;

6. MVP program (Lambert, (1997) in Practical Application of Computer-Aided Drug Design, (Charifson, ed.) Marcel-Dekker, New York, pp. 243-303); and

7. LUDI™ program (Bohm, (1992) J. Comput. Aid. Mol. Des. 6: 61-78), which is available from Molecular Simulations, Inc., San Diego, Calif.

Once suitable chemical entities or fragments have been selected, they can be assembled into a single compound or modulator. Assembly can proceed by visual inspection of the relationship of the fragments to each other on the three-dimensional image displayed on a computer screen in relation to the structure coordinates of a LXR LBD. Manual model building using software such as QUANTA™ or SYBYL™ typically follows.

Useful programs to aid one of ordinary skill in the art in connecting the individual chemical entities or fragments include:

1. CAVEAT™ program (Bartlett et al., (1989) Special Pub., Royal Chem. Soc. 78: 182-96), which is available from the University of California, Berkeley, Calif.;

2. 3D database systems, such as MACCS-3D™ system program, which is available from MDL Information Systems, San Leandro, Calif. This area is reviewed in Martin, (1992) J. Med. Chem. 35: 2145-54; and

3. HOOK™ program (Eisen et al., (1994). Proteins 19: 199-221), which is available from Molecular Simulations, Inc., San Diego, Calif.

Instead of proceeding to build a LXR LBD modulator (preferably a hLXR LBD modulator) in a step-wise fashion one fragment or chemical entity at a time as described above, modulatory or other binding compounds can be designed as a whole or de novo using the structural coordinates of a crystalline LXR LBD polypeptide of the present invention and either an empty binding site or optionally including some portion(s) of a known modulator(s). Applicable methods can employ the following software programs:

1. LUDI™ program (Bohm, (1992) J. Comput Aid. Mol. Des. 6: 61-78), which is available from Molecular Simulations, Inc., San Diego, Calif.;

2. LEGEND™ program (Nishibata & Itai, (1991) Tetrahedron 47: 8985); and

3. LEAPFROG™, which is available from Tripos Associates, St. Louis, Mo.

Other molecular modeling techniques can also be employed in accordance with this invention. See, e.g., Cohen et al., (1990) J. Med. Chem. 33: 883-94. See also, Navia & Murcko, (1992) Curr. Opin. Struc. Biol. 2: 202-10; U.S. Pat. No. 6,008,033, herein incorporated by reference.

Once a compound has been designed or selected by the above methods, the efficiency with which that compound can bind to a LXR LBD can be tested and optimized by computational evaluation. By way of particular example, a compound that has been designed or selected to function as a LXR LBD modulator should also preferably traverse a volume not overlapping that occupied by the binding site when it is bound to its native ligand. Additionally, an effective LXR LBD modulator should preferably demonstrate a relatively small difference in energy between its bound and free states (i.e., a small deformation energy of binding). Thus, the most efficient LXR LBD modulators should preferably be designed with a deformation energy of binding of not greater than about 10 kcal/mole, and preferably, not greater than 7 kcal/mole. It is possible for LXR LBD modulators to interact with the polypeptide in more than one conformation that is similar in overall binding energy. In those cases, the deformation energy of binding is taken to be the difference between the energy of the free compound and the average energy of the conformations observed when the modulator binds to the polypeptide.

A compound designed or selected as binding to a LXR polypeptide (preferably a hLXR LBD polypeptide) can be further computationally optimized so that in its bound state it would preferably lack repulsive electrostatic interaction with the target polypeptide. Such non-complementary (e.g., electrostatic) interactions include repulsive charge-charge, dipole-dipole and charge-dipole interactions. Specifically, the sum of all electrostatic interactions between the modulator and the polypeptide when the modulator is bound to a LXR LBD preferably make a neutral or favorable contribution to the enthalpy of binding.

Specific computer software is available in the art to evaluate compound deformation energy and electrostatic interaction. Examples of programs designed for such uses include:

1. Gaussian 98™, which is available from Gaussian, Inc., Pittsburgh, Pa.;

2. AMBER™ program, version 6.0, which is available from the University of California at San Francisco;

3. QUANTA™ program, which is available from Molecular Simulations, Inc., San Diego, Calif.;

4. CHARMm® program, which is available from Molecular Simulations, Inc., San Diego, Calif.; and

4. INSIGHT II® program, which is available from Molecular Simulations, Inc., San Diego, Calif.

These programs can be implemented using a suitable computer system. Other hardware systems and software packages will be apparent to those skilled in the art after review of the disclosure of the present invention presented herein.

Once a LXR LBD modulating compound has been optimally selected or designed, as described above, substitutions can then be made in some of its atoms or side groups in order to improve or modify its binding properties. Generally, initial substitutions are conservative, i.e., the replacement group will have approximately the same size, shape, hydrophobicity and charge as the original group. It should, of course, be understood that components known in the art to alter conformation should be avoided. Such substituted chemical compounds can then be analyzed for efficiency of fit to a LXR LBD binding site using the same computer-based approaches described in detail above.

VIII.B. Method of Screening for Chemical and Biological Modulators of the Biological Activity of LXR

A candidate substance identified according to a screening assay of LXR present invention has an ability to modulate the biological activity of a LXR polypeptide or a LXR LBD polypeptide. In a preferred embodiment, such a candidate compound can have utility in the treatment of disorders and conditions associated with the biological activity of a LXR or a LXR LBD polypeptide, including, but not limited to reducing serum levels of cholesterol and reducing arterial plaque formation and accumulation.

In a cell-free system, the method comprises the steps of establishing a control system comprising a LXR polypeptide and a ligand which is capable of binding to the polypeptide; establishing a test system comprising a LXR polypeptide, the ligand, and a candidate compound; and determining whether the candidate compound modulates the activity of the polypeptide by comparison of the test and control systems. A representative ligand comprises N-(2,2,2-trifluoroethyl)-N-[4-(2,2,2-trifluoro-1-hydroxy-1-trifluoromethyl-ethyl)-phenyl]-benzenesulfonamide, EPC, a peptide or a small molecule, and in this embodiment, the biological activity or property screened includes binding affinity.

In another embodiment of the invention, a form of a LXR polypeptide or a catalytic or immunogenic fragment or oligopeptide thereof, can be used for screening libraries of compounds in any of a variety of drug screening techniques. The fragment employed in such a screening can be affixed to a solid support. The formation of binding complexes, between a LXR polypeptide and the agent being tested, will be detected. In a preferred embodiment, the LXR polypeptide has an amino acid sequence of SEQ ID NO: 4. When a LXR LBD polypeptide is employed, a preferred embodiment includes a LXR polypeptide having the amino acid sequence of SEQ ID NO: 8.

Another technique for drug screening which can be used provides for high throughput screening of compounds having suitable binding affinity to the protein of interest as described in published PCT application WO 84/03564, herein incorporated by reference. In this method, as applied to a polypeptide of the present invention, large numbers of different small test compounds are synthesized on a solid substrate, such as plastic pins or some other surface. The test compounds are reacted with the polypeptide, or fragments thereof. Bound polypeptide is then detected by methods well known to those of skill in the art. The polypeptide can also be placed directly onto plates for use in the aforementioned drug screening techniques.

In yet another embodiment, a method of screening for a modulator of a LXR polypeptide or a LXR LBD polypeptide comprises: providing a library of test samples; contacting a LXR polypeptide or a LXR LBD polypeptide with each test sample; detecting an interaction between a test sample and a LXR polypeptide or a LXR LBD polypeptide; identifying a test sample that interacts with a LXR polypeptide or a LXR LBD polypeptide; and isolating a test sample that interacts with a LXR polypeptide or a LXR LBD polypeptide.

In each of the foregoing embodiments, an interaction can be detected spectrophotometrically, radiologically or immunologically. An interaction between a LXR polypeptide or a LXR LBD polypeptide and a test sample can also be quantified using methodology known to those of skill in the art. In another embodiment, the LXR polypeptide and the LXR LBD is in crystalline form.

In accordance with the present invention there is also provided a rapid and high throughput screening method that relies on the methods described above. This screening method comprises separately contacting each of a plurality of substantially identical samples with a LXR polypeptide or a LXR LBD and detecting a resulting binding complex. In such a screening method the plurality of samples preferably comprises more than about 10⁴ samples, and more preferably comprises more than about 5×10⁴ samples.

VIII.C. Method of Identifying Compounds Which Inhibit Ligand Binding

Using the crystal structures and ligand orientations, disclosed for the first time in the present invention, it is possible to design test compounds that inhibit binding of ligands normally bound by a LXR polypeptide.

In one aspect of the present invention, an assay method for identifying a compound that inhibits binding of a ligand to a LXR polypeptide is disclosed. A known ligand of LXR can be used in the assay method as the ligand against which the inhibition by a test compound is gauged. N-(2,2,2-trifluoroethyl)-N-[4-(2,2,2-trifluoro-1-hydroxy-1-trifluoromethyl-ethyl)-phenyl]-benzenesulfonamide, EPC, a SRC-dervied peptide, and combinations thereof, are preferred ligands in the assay method. The method comprises (a) incubating a LXR polypeptide with a ligand in the presence of a test inhibitor compound; (b) determining an amount of ligand that is bound to the LXR polypeptide, wherein decreased binding of ligand to the LXR polypeptide in the presence of the test inhibitor compound relative to binding in the absence of the test inhibitor compound is indicative of inhibition; and (c) identifying the test compound as an inhibitor of ligand binding if decreased ligand binding is observed. Preferably, the ligand is N-(2,2,2-trifluoroethyl)-N-[4-(2,2,2-trifluoro-1-hydroxy-1-trifluoromethyl-ethyl)-phenyl]-benzenesulfonamide, EPC, a SRC-dervied peptide, and combinations thereof.

In another aspect of the present invention, the disclosed assay method can be employed in the structural refinement of candidate LXR inhibitors. For example, multiple rounds of optimization can be followed by gradual structural changes in a strategy of inhibitor design. A strategy such as this is made possible by the disclosure of the coordinates of the LXR LBD and the disclosure of the orientation position of ligands of LXR, namely N-(2,2,2-trifluoroethyl)-N-[4-(2,2,2-trifluoro-1-hydroxy-1-trifluoromethyl-ethyl)-phenyl]-benzenesulfonamide, EPC, a SRC-dervied peptide, and combinations thereof.

IX. Design, Preparation and Structural Analysis of LXR and LXR LBD Mutants and Structural Equivalents

The present invention provides for the generation of LXR and LXR mutants (preferably hLXR and hLXR LBD mutants), and the ability to solve the crystal structures of those that crystallize. More particularly, through the provision of the three-dimensional structure of a LXR LBD, desirable sites for mutation can be identified, based on analysis of the three-dimensional LXR LBD structure coordinates provided herein.

The structure coordinates of a LXR LBD provided in accordance with the present invention also facilitate the identification of related proteins or enzymes analogous to LXR in function, structure or both, (for example, a mouse LXR), which can lead to novel therapeutic modes for treating or preventing a range of disease states, such as minimization of serum cholesterol levels and preventing the accumulation of cholesterol plaques.

IX.A. Sterically Similar Compounds

A further aspect of the present invention is that sterically similar compounds can be formulated to mimic the key portions of a LXR LBD structure. Such compounds are functional equivalents. The generation of a structural functional equivalent can be achieved by the techniques of modeling and chemical design known to those of skill in the art and described herein. Modeling and chemical design of LXR and LXR LBD structural equivalents can be based on the structure coordinates of a crystalline LXR LBD polypeptide of the present invention. It will be understood that all such sterically similar constructs fall within the scope of the present invention.

IX.B. LXR Polypeptides

The generation of chimeric LXR polypeptides is also an aspect of the present invention. Such a chimeric polypeptide can comprise a LXR LBD polypeptide or a portion of a LXR LBD, (e.g. a hLXR LBD) which is fused to a candidate polypeptide or a suitable region of the candidate polypeptide, for example a LXR expressed in mouse or other species. Throughout the present disclosure it is intended that the term “mutant” encompass not only mutants of a LXR LBD polypeptide but chimeric proteins generated using a LXR LBD as well. It is thus intended that the following discussion of mutant LXR LBDs apply mutatis mutandis to chimeric LXR and LXR LBD polypeptides and to structural equivalents thereof.

In accordance with the present invention, a mutation can be directed to a particular site or combination of sites of a wild-type LXR LBD. For example, an accessory binding site or the binding cavity can be chosen for mutagenesis. Similarly, a residue having a location on, at or near the surface of the polypeptide can be replaced, resulting in an altered surface charge of one or more charge units, as compared to the wild-type LXR and LXR LBD. Alternatively, an amino acid residue in a LXR or a LXR LBD can be chosen for replacement based on its hydrophilic or hydrophobic characteristics.

Such mutants can be characterized by any one of several different properties as compared with the wild-type LXR LBD. For example, such mutants can have an altered surface charge of one or more charge units, or can have an increase in overall stability. Other mutants can have altered substrate specificity in comparison with, or a higher specific activity than, a wild-type LXR or LXR LBD.

LXR and LXR LBD mutants of the present invention can be generated in a number of ways. For example, the wild-type sequence of a LXR or a LXR LBD can be mutated at those sites identified using this invention as desirable for mutation, by means of oligonucleotide-directed mutagenesis or other conventional methods, such as deletion. Alternatively, mutants of a LXR or a LXR LBD can be generated by the site-specific replacement of a particular amino acid with an unnaturally occurring amino acid. In addition, LXR or LXR LBD mutants can be generated through replacement of an amino acid residue, for example, a particular cysteine or methionine residue, with selenocysteine or selenomethionine. This can be achieved by growing a host organism capable of expressing either the wild-type or mutant polypeptide on a growth medium depleted of either natural cysteine or methionine (or both) but enriched in selenocysteine or selenomethionine (or both).

Mutations can be introduced into a DNA sequence coding for a LXR or a LXR LBD using synthetic oligonucleotides. These oligonucleotides contain nucleotide sequences flanking the desired mutation sites. Mutations can be generated in the full-length DNA sequence of a LXR or a LXR LBD or in any sequence coding for polypeptide fragments of a LXR or a LXR LBD.

According to the present invention, a mutated LXR or LXR LBD DNA sequence produced by the methods described above, or any alternative methods known in the art, can be expressed using an expression vector. An expression vector, as is well known to those of skill in the art, typically includes elements that permit autonomous replication in a host cell independent of the host genome, and one or more phenotypic markers for selection purposes. Either prior to or after insertion of the DNA sequences surrounding the desired LXR or LXR LBD mutant coding sequence, an expression vector also will include control sequences encoding a promoter, operator, ribosome binding site, translation initiation signal, and, optionally, a repressor gene or various activator genes and a signal for termination. In some embodiments, where secretion of the produced mutant is desired, nucleotides encoding a “signal sequence” can be inserted prior to a LXR or a LXR LBD mutant coding sequence. For expression under the direction of the control sequences, a desired DNA sequence must be operatively linked to the control sequences; that is, the sequence must have an appropriate start signal in front of the DNA sequence encoding the LXR or LXR LBD mutant, and the correct reading frame to permit expression of that sequence under the control of the control sequences and production of the desired product encoded by that LXR or LXR LBD sequence must be maintained.

Any of a wide variety of well-known available expression vectors can be useful to express a mutated LXR or LXR LBD coding sequences of this invention and generated as described in Laboratory Example 3. These expression vectors can be used in the techniques disclosed in Laboratory Examples 1 and 3 and can include, for example, vectors comprising segments of chromosomal, non-chromosomal and synthetic DNA sequences, such as various known derivatives of SV40, known bacterial plasmids, e.g., plasmids from E. coli including col E1, pCR1, pBR322, pMB9 and their derivatives, wider host range plasmids, e.g., RP4, phage DNAs, e.g., the numerous derivatives of phage λ, e.g., NM 989, and other DNA phages, e.g., M13 and filamentous single stranded DNA phages, yeast plasmids and vectors derived from combinations of plasmids and phage DNAs, such as plasmids which have been modified to employ phage DNA or other expression control sequences. In a preferred embodiment of this invention, the E. coli vector pRSETA, including a T7-based expression system, is employed.

In addition, any of a wide variety of expression control sequences-sequences that control the expression of a DNA sequence when operatively linked to it—can be used in these vectors to express the mutated DNA sequences according to this invention. Such useful expression control sequences, include, for example, the early and late promoters of SV40 for animal cells, the lac system, the trp system the TAC or TRC system, the major operator and promoter regions of phage λ, the control regions of fd coat protein, all for E. coli, the promoter for 3-phosphoglycerate kinase or other glycolytic enzymes, the promoters of acid phosphatase, e.g., Pho5, the promoters of the yeast α-mating factors for yeast, and other sequences known to control the expression of genes of prokaryotic or eukaryotic cells or their viruses, and various combinations thereof.

A wide variety of hosts are also useful for producing mutated LXR and LXR LBD polypeptides according to this invention. These hosts include, for example, bacteria, such as E. coli, Bacillus and Streptomyces, fungi, such as yeasts, and animal cells, such as CHO and COS-1 cells, plant cells, insect cells, such as Sf9 cells, and transgenic host cells.

It should be understood that not all expression vectors and expression systems function in the same way to express mutated DNA sequences of this invention, and to produce modified LXR and LXR LBD polypeptides or LXR or LXR LBD mutants. Neither do all hosts function equally well with the same expression system. One of skill in the art can, however, make a selection among these vectors, expression control sequences and hosts without undue experimentation and without departing from the scope of this invention. For example, an important consideration in selecting a vector will be the ability of the vector to replicate in a given host. The copy number of the vector, the ability to control that copy number, and the expression of any other proteins encoded by the vector, such as antibiotic markers, should also be considered.

In selecting an expression control sequence, a variety of factors should also be considered. These include, for example, the relative strength of the system, its controllability and its compatibility with the DNA sequence encoding a modified LXR or LXR LBD polypeptide of this invention, with particular regard to the formation of potential secondary and tertiary structures.

Hosts should be selected by consideration of their compatibility with the chosen vector, the toxicity of a modified LXR or LXR LBD to them, their ability to express mature products, their ability to fold proteins correctly, their fermentation requirements (if any), the ease of purification of a modified LXR or LXR LBD and safety. Within these parameters, one of skill in the art can select various vector/expression control system/host combinations that will produce useful amounts of a mutant LXR or LXR LBD. A mutant LXR or LXR LBD produced in these systems can be purified by a variety of conventional steps and strategies, including those used to purify the wild-type LXR or LXR LBD.

Once a LXR LBD mutation(s) has been generated in the desired location, such as a ligand binding or dimerization site, the mutants can be tested for any one of several properties of interest. For example, mutants can be screened for an altered charge at physiological pH. This is determined by measuring the mutant LXR or LXR LBD isoelectric point (pI) and comparing the observed value with that of the wild-type parent. Isoelectric point can be measured by gel-electrophoresis according to the method of Wellner (Wellner, (1971) Anal. Chem. 43: 597). A mutant LXR or LXR LBD polypeptide containing a replacement amino acid located at the surface of the enzyme, as provided by the structural information of this invention, can lead to an altered surface charge and an altered pl.

IX.C. Generation of an Engineered LXR or LXR LBD Mutant

In another aspect of the present invention, a unique LXR or LXR LBD polypeptide can be generated. Such a mutant can facilitate purification and can facilitate the study of the ligand binding abilities of a LXR polypeptide.

As used in the following discussion, the terms “engineered LXR”, “engineered LXR LDB”, “LXR mutant”, and “LXR LBD mutant” refers to polypeptides having amino acid sequences which contain at least one mutation in the wild-type sequence. The terms also refer to LXR and LXR LBD polypeptides which are capable of exerting a biological effect in that they comprise all or a part of the amino acid sequence of an engineered LXR or LXR LBD mutant polypeptide of the present invention, or cross-react with antibodies raised against an engineered LXR or LXR LBD mutant polypeptide, or retain all or some or an enhanced degree of the biological activity of the engineered LXR or LXR LBD mutant amino acid sequence or protein. Such biological activity can include ligand binding (e.g. EPC and N-(2,2,2-trifluoroethyl)-N-[4-(2,2,2-trifluoro-1-hydroxy-1-trifluoromethyl-ethyl)-phenyl]-benzenesulfonamide) and co-factor binding (e.g. binding a SRC peptide), as well as reducing serum cholesterol levels and reducing accumulation of cholesterol plaques.

The terms “engineered LXR LBD” and “LXR LBD mutant” also includes analogs of an engineered LXR LBD or LXR LBD mutant polypeptide. By “analog” is intended that a DNA or polypeptide sequence can contain alterations relative to the sequences disclosed herein, yet retain all or some or an enhanced degree of the biological activity of those sequences. Analogs can be derived from genomic nucleotide sequences or from other organisms, or can be created synthetically. Those of skill in the art will appreciate that other analogs, as yet undisclosed or undiscovered, can be used to design and/or construct LXR LBD or LXR LBD mutant analogs. There is no need for an engineered LXR LBD or LXR LBD mutant polypeptide to comprise all or substantially all of the amino acid sequence of SEQ ID NOs: 6 or 8. Shorter or longer sequences are anticipated to be of use in the invention; shorter sequences are herein referred to as “segments”. Thus, the terms “engineered LXR LBD” and “LXR LBD mutant” also includes fusion, chimeric or recombinant engineered LXR LBD or LXR LBD mutant polypeptides and proteins comprising sequences of the present invention. Methods of preparing such proteins are disclosed herein above and are known in the art.

IX.D. Sequence Similarity and Identity

As used herein, the term “substantially similar” means that a particular sequence varies from nucleic acid sequence of SEQ ID NOs: 1, 3, 5, and 7, or the amino acid sequence of SEQ ID NOs: 2, 4, 6, 8 and 9 by one or more deletions, substitutions, or additions, the net effect of which is to retain at least some of biological activity of the natural gene, gene product, or sequence. Such sequences include “mutant” or “polymorphic” sequences, or sequences in Which the biological activity and/or the physical properties are altered to some degree but retains at least some or an enhanced degree of the original biological activity and/or physical properties. In determining nucleic acid sequences, all subject nucleic acid sequences capable of encoding substantially similar amino acid sequences are considered to be substantially similar to a reference nucleic acid sequence, regardless of differences in codon sequences or substitution of equivalent amino acids to create biologically functional equivalents.

IX.D.1. Sequences that are Substantially Identical to an Engineered LXR or LXR LBD Mutant Sequence of the Present Invention

Nucleic acids that are substantially identical to a nucleic acid sequence of an engineered LXR or LXR LBD mutant of the present invention, e.g. allelic variants, genetically altered versions of the gene, etc., bind to an engineered LXR or LXR LBD mutant sequence under stringent hybridization conditions. By using probes, particularly labeled probes of DNA sequences, one can isolate homologous or related genes. The source of homologous genes can be any species, e.g. primate species; rodents, such as rats and mice, canines, felines, bovines, equines, yeast, nematodes, etc.

Between mammalian species, e.g. human and mouse, homologs have substantial sequence similarity, i.e. at least 75% sequence identity between nucleotide sequences. Sequence similarity is calculated based on a reference sequence, which can be a subset of a larger sequence, such as a conserved motif, coding region, flanking region, etc. A reference sequence will usually be at least about 18 nucleotides(nt) long, more usually at least about 30 nt long, and can extend to the complete sequence that is being compared. Algorithms for sequence analysis are known in the art, such as BLAST, described in Altschul et al., (1990) J. Mol. Biol. 215: 403-10.

Percent identity or percent similarity of a DNA or peptide sequence can be determined, for example, by comparing sequence information using the GAP computer program, available from the University of Wisconsin Geneticist Computer Group. The GAP program utilizes the alignment method of Needleman et al., (1970) J. Mol. Biol. 48: 443, as revised by Smith et al., (1981) Adv. Appl. Math. 2:482. Briefly, the GAP program defines similarity as the number of aligned symbols (i.e., nucleotides or amino acids) that are similar, divided by the total number of symbols in the shorter of the two sequences. The preferred parameters for the GAP program are the default parameters, which do not impose a penalty for end gaps. See, e.g., Schwartz et al. (eds.), (1979), Atlas of Protein Sequence and Structure, National Biomedical Research Foundation, pp. 357-358, and Gribskov et al., (1986) Nucl. Acids. Res. 14: 6745.

The term “similarity” is contrasted with the term “identity”. Similarity is defined as above; “identity”, however, means a nucleic acid or amino acid sequence having the same amino acid at the same relative position in a given family member of a gene family. Homology and similarity are generally viewed as broader terms than the term identity. Biochemically similar amino acids, for example leucine/isoleucine or glutamate/aspartate, can be present at the same position—these are not identical per se, but are biochemically “similar.” As disclosed herein, these are referred to as conservative differences or conservative substitutions. This differs from a conservative mutation at the DNA level, which changes the nucleotide sequence without making a change in the encoded amino acid, e.g. TCC to TCA, both of which encode serine.

As used herein, DNA analog sequences are “substantially identical” to specific DNA sequences disclosed herein if: (a) the DNA analog sequence is derived from coding regions of the nucleic acid sequence shown in SEQ ID NOs: 1, 3, 5 and 7; or (b) the DNA analog sequence is capable of hybridization with DNA sequences of (a) under stringent conditions and which encode a biologically active LXR or LXR LBD gene product; or (c) the DNA sequences are degenerate as a result of alternative genetic code to the DNA analog sequences defined in (a) and/or (b). Substantially identical analog proteins and nucleic acids will have between about 70% and 80%, preferably between about 81% to about 90% or even more preferably between about 91% and 99% sequence identity with the corresponding sequence of the native protein or nucleic acid. Sequences having lesser degrees of identity but comparable biological activity are considered to be equivalents.

As used herein, “stringent conditions” means conditions of high stringency, for example 6×SSC, 0.2% polyvinylpyrrolidone, 0.2% Ficoll, 0.2% bovine serum albumin, 0.1% sodium dodecyl sulfate, 100 μg/ml salmon sperm DNA and 15% formamide at 68° C. For the purposes of specifying additional conditions of high stringency, preferred conditions are salt concentration of about 200 mM and temperature of about 45° C. One example of such stringent conditions is hybridization at 4×SSC, at 65° C., followed by a washing in 0.1×SSC at 65° C. for one hour. Another exemplary stringent hybridization scheme uses 50% formamide, 4×SSC at 42° C.

In contrast, nucleic acids having sequence similarity are detected by hybridization under lower stringency conditions. Thus, sequence identity can be determined by hybridization under lower stringency conditions, for example, at 50° C. or higher and 0.1×SSC (9 mM NaCl/0.9 mM sodium citrate) and the sequences will remain bound when subjected to washing at 55° C. in 1×SSC.

IX.D.2. Complementarity and Hybridization to an Engineered LXR or LXR LBD Mutant Sequence

As used herein, the term “complementary sequences” means nucleic acid sequences that are base-paired according to the standard Watson-Crick complementarity rules. The present invention also encompasses the use of nucleotide segments that are complementary to the sequences of the present invention.

Hybridization can also be used for assessing complementary sequences and/or isolating complementary nucleotide sequences. As discussed above, nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringent temperature conditions will generally include temperatures in excess of about 30° C., typically in excess of about 37° C., and preferably in excess of about 45° C. Stringent salt conditions will ordinarily be less than about 1,000 mM, typically less than about 500 mM, and preferably less than about 200 mM. However, the combination of parameters is much more important than the measure of any single parameter. See, e.g., Wetmur & Davidson, (1968) J. Mol. Biol. 31: 349-70. Determining appropriate hybridization conditions to identify and/or isolate sequences containing high levels of homology is well known in the art. See, e.g., Sambrook et al., (1989) Molecular Cloning: A Laboratory Manual, Cold Spring Harbor, N.Y.

IX.D.3. Functional Equivalents of an Engineered LXR or LXR LBD Mutant Nucleic Acid Sequence of the Present Invention

As used herein, the term “functionally equivalent codon” is used to refer to codons that encode the same amino acid, such as the ACG and AGU codons for serine. LXR or LXR LBD-encoding nucleic acid sequences comprising SEQ ID NOs: 1, 3, 5 and 7, which have functionally equivalent codons, are covered by the present invention. Thus, when referring to the sequence examples presented in SEQ ID NOs: 2, 4, 6 and 8, applicants contemplate substitution of functionally equivalent codons into the sequence example of SEQ ID NOs: 1, 3, 5 and 7. Thus, applicants are in possession of amino acid and nucleic acids sequences which include such substitutions but which are not set forth herein in their entirety for convenience.

It will also be understood by those of skill in the art that amino acid and nucleic acid sequences can include additional residues, such as additional N- or C-terminal amino acids or 5′ or 3′ nucleic acid sequences, and yet still be essentially as set forth in one of the sequences disclosed herein, so long as the sequence retains biological protein activity where polypeptide expression is concerned. The addition of terminal sequences particularly applies to nucleic acid sequences which can, for example, include various non-coding sequences flanking either of the 5′ or 3′ portions of the coding region or can include various internal sequences, i.e., introns, which are known to occur within genes.

IX.D.4. Biological Equivalents

The present invention envisions and includes biological equivalents of an engineered LXR or LXR LBD mutant polypeptide of the present invention. The term “biological equivalent” refers to proteins having amino acid sequences which are substantially identical to the amino acid sequence of an engineered LXR LBD mutant of the present invention and which are capable of exerting a biological effect in that they are capable of binding DNA moieties or cross-reacting with anti-LXR or LXR LBD mutant antibodies raised against an engineered mutant LXR or LXR LBD polypeptide of the present invention.

For example, certain amino acids can be substituted for other amino acids in a protein structure without appreciable loss of interactive capacity with, for example, structures in the nucleus of a cell. Since it is the interactive capacity and nature of a protein that defines that protein's biological functional activity, certain amino acid sequence substitutions can be made in a protein sequence (or the nucleic acid sequence encoding it) to obtain a protein with the same, enhanced, or antagonistic properties. Such properties can be achieved by interaction with the normal targets of the protein, but this need not be the case, and the biological activity of the invention is not limited to a particular mechanism of action. It is thus in accordance with the present invention that various changes can be made in the amino acid sequence of an engineered LXR or LXR LBD mutant polypeptide of the present invention or its underlying nucleic acid sequence without appreciable loss of biological utility or activity.

Biologically equivalent polypeptides, as used herein, are polypeptides in which certain, but not most or all, of the amino acids can be substituted. Thus, when referring to the sequence examples presented in SEQ ID NOs: 2, 4, 6, 8 and 9, applicants envision substitution of codons that encode biologically equivalent amino acids, as described herein, into the sequence examples of SEQ ID NOs: 1, 3, 5 and 7, respectively. Thus, applicants are in possession of amino acid and nucleic acids sequences which include such substitutions but which are not set forth herein in their entirety for convenience.

Alternatively, functionally equivalent proteins or peptides can be created via the application of recombinant DNA technology, in which changes in the protein structure can be engineered, based on considerations of the properties of the amino acids being exchanged, e.g. substitution of Ile for Leu. Changes designed by man can be introduced through the application of site-directed mutagenesis techniques, e.g., to introduce improvements to the antigenicity of the protein or to test an engineered LXR or LXR LBD mutant polypeptide of the present invention for its ability to modulate DNA-binding, ligand-binding or other activity, at the molecular level.

Amino acid substitutions, such as those that might be employed in modifying an engineered LXR or LXR LBD mutant polypeptide of the present invention are generally, but not necessarily, based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all of similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents. Other biologically functionally equivalent changes will be appreciated by those of skill in the art. It is implicit in the above discussion, however, that one of skill in the art can appreciate that a radical, rather than a conservative substitution is warranted in a given situation. Non-conservative substitutions in engineered mutant LXR or LXR LBD polypeptides of the present invention are also an aspect of the present invention.

In making biologically functional equivalent amino acid substitutions, the hydropathic index of amino acids can be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine (+2.5); methionine (+1.9); alanine (+1.8); glycine (−-0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte & Doolittle, (1982), J. Mol. Biol. 157: 105-132, incorporated herein by reference). It is known that certain amino acids can be substituted for other amino acids having a similar hydropathic index or score and still retain a similar biological activity. In making changes based upon the hydropathic index, the substitution of amino acids whose hydropathic indices are within ±2 of the original value is preferred, those which are within ±1 of the original value are particularly preferred, and those within ±0.5 of the original value are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with its immunogenicity and antigenicity, i.e. with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0±1); glutamate (+3.0+1); serine (+0.3); asparagine (+0.2); glutamine (+0.2); glycine (0); threonine (−0.4); proline (−0.5+1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 of the original value is preferred, those which are within ±1 of the original value are particularly preferred, and those within ±0.5 of the original value are even more particularly preferred.

While discussion has focused on functionally equivalent polypeptides arising from amino acid changes, it will be appreciated that these changes can be effected by alteration of the encoding DNA, taking into consideration also that the genetic code is degenerate and that two or more codons can code for the same amino acid.

Thus, it will also be understood that this invention is not limited to the particular amino acid and nucleic acid sequences of SEQ ID NOs: 1-9. Recombinant vectors and isolated DNA segments can therefore variously include an engineered LXR or LXR LBD polypeptide-encoding region itself, include coding regions bearing selected alterations or modifications in the basic coding region, or include larger polypeptides which nevertheless comprise a LXR or LXR LBD polypeptide-encoding regions or can encode biologically functional equivalent proteins or polypeptides which have variant amino acid sequences. Biological activity of an engineered LXR or LXR LBD polypeptide can be determined, for example, by ligand binding assays known to those of skill in the art.

The nucleic acid segments of the present invention, regardless of the length of the coding sequence itself, can be combined with other DNA sequences, such as promoters, enhancers, polyadenylation signals, additional restriction enzyme sites, multiple cloning sites, other coding segments, and the like, such that their overall length can vary considerably. It is therefore contemplated that a nucleic acid fragment of almost any length can be employed, with the total length preferably being limited by the ease of preparation and use in the intended recombinant DNA protocol. For example, nucleic acid fragments can be prepared which include a short stretch complementary to a nucleic acid sequence set forth in SEQ ID NOs: 1, 3, 5 and 7, such as about 10 nucleotides, and which are up to 10,000 or 5,000 base pairs in length. DNA segments with total lengths of about 4,000, 3,000, 2,000, 1,000, 500, 200, 100, and about 50 base pairs in length are also useful.

The DNA segments of the present invention encompass biologically functional equivalents of engineered LXR or LXR LBD polypeptides. Such sequences can rise as a consequence of codon redundancy and functional equivalency that are known to occur naturally within nucleic acid sequences and the proteins thus encoded. Alternatively, functionally equivalent proteins or polypeptides can be created via the application of recombinant DNA technology, in which changes in the protein structure can be engineered, based on considerations of the properties of the amino acids being exchanged. Changes can be introduced through the application of site-directed mutagenesis techniques, e.g., to introduce improvements to the antigenicity of the protein or to test variants of an engineered LXR or LXR LBD mutant of the present invention in order to examine the degree of ligand binding activity, or other activity at the molecular level. Various site-directed mutagenesis techniques are known to those of skill in the art and can be employed in the present invention.

The invention further encompasses fusion proteins and peptides wherein an engineered LXR or LXR LBD mutant coding region of the present invention is aligned within the same expression unit with other proteins or peptides having desired functions, such as for purification or immunodetection purposes.

Recombinant vectors form important further aspects of the present invention. Particularly useful vectors are those in which the coding portion of the DNA segment is positioned under the control of a promoter. The promoter can be that naturally associated with a LXR gene, as can be obtained by isolating the 5′ non-coding sequences located upstream of the coding segment or exon, for example, using recombinant cloning and/or PCR technology and/or other methods known in the art, in conjunction with the compositions disclosed herein.

In other embodiments, certain advantages will be gained by positioning the coding DNA segment under the control of a recombinant, or heterologous, promoter. As used herein, a recombinant or heterologous promoter is a promoter that is not normally associated with a LXR gene in its natural environment. Such promoters can include promoters isolated from bacterial, viral, eukaryotic, or mammalian cells. Naturally, it will be important to employ a promoter that effectively directs the expression of the DNA segment in the cell type chosen for expression. The use of promoter and cell type combinations for protein expression is generally known to those of skill in the art of molecular biology (See, e.g., Sambrook et al., (1989) Molecular Cloning: A Laboratory Manual, Cold Spring Harbor Laboratory, New York, specifically incorporated herein by reference). The promoters employed can be constitutive or inducible and can be used under the appropriate conditions to direct high level expression of the introduced DNA segment, such as is advantageous in the large-scale production of recombinant proteins or peptides. One preferred promoter system contemplated for use in high-level expression is a T7 promoter-based system.

X. Uses of LXR Mutants

The LXR mutants disclosed herein have a variety of applications, including in the screening of components for LXR activation using the cell-free reporter gene assay methods disclosed herein above, and using whole animal models. The LXR mutants can also be used in cell-free, cell-based and whole animal assay methods for bioavailability of compounds and for toxicology analysis. Additionally, LXR mutants can be employed in crystallizations, screening for changes in ligand activation, screening for species-specific changes in ligand activation and screening for changes in oligomerization state both with and without ligand.

XI. The Role of the Three-Dimensional Structure of the hLXR LDB in Solving Additional LXR Crystals

Because polypeptides can crystallize in more than one crystal form, the structural coordinates of a hLXR LBD, or portions thereof, as provided by the present invention, are particularly useful in solving the structure of other crystal forms of hLXR and the crystalline forms of other LXRs. The coordinates provided in the present invention can also be used to solve the structure of LXR or LXR LBD mutants (such as those described above), LXR LDB co-complexes, or of the crystalline form of any other protein with significant amino acid sequence homology to any functional domain of LXR.

XI.A. Determining the Three-Dimensional Structure of a Polypeptide Using the Three-Dimensional Structure of the hLXR LBD as a Template in Molecular Replacement

One method that can be employed for the purpose of solving additional LXR crystal structures is molecular replacement. See generally, Rossmann (ed), (1972) The Molecular Replacement Method, Gordon & Breach, New York. In the molecular replacement method, the unknown crystal structure, whether it is another crystal form of a LXR or a LXR LBD, (i.e. a LXR or a LXR LBD mutant), or a LXR or a LXR LBD polypeptide complexed with another compound (a “co-complex”), or the crystal of some other protein with significant amino acid sequence homology to any functional region of the a hLXR LBD, can be determined using the hLXR LBD structure coordinates provided in Tables 2-4. This method provides an accurate structural form for the unknown crystal more quickly and efficiently than attempting to determine such information ab initio.

In addition, in accordance with this invention, LXR or LXR mutants (preferably hLXR or hLXR LBD mutants) can be crystallized in complex with known modulators. The crystal structures of a series of such complexes can then be solved by molecular replacement and compared with that of wild-type hLXR or the wild-type hLXR LBD. Potential sites for modification within the various binding sites of the enzyme can thus be identified. This information provides an additional tool for determining the most efficient binding interactions, for example, increased hydrophobic interactions, between the hLXR LBD and a chemical entity or compound.

All of the complexes referred to in the present disclosure can be studied using X-ray diffraction techniques (See, e.g., Blundell & Johnson (1985) Method. Enzymol. 114A & 115B, (Wyckoff et al., eds.), Academic Press) and can be refined using computer software, such as the X-PLOR™ program (Brünger, (1992) X-PLOR, Version 3.1. A System for X-ray Crystallography and NMR, Yale University Press, New Haven, Conn.; X-PLOR is available from Molecular Simulations, Inc., San Diego, Calif.). This information can thus be used to optimize known classes of LXR and LXR LBD modulators, and more importantly, to design and synthesize novel classes of LXR and LXR LBD modulators.

XII. Conclusions

To aid further drug discovery efforts, the interactions involved in the binding and activation of LXR have been identified. In one aspect, the present invention discloses the atomic structures of the complexes of LXRβ with the activator T317, and the complex of LXRβ with EPC and the second LXXLL (SEQ ID NO: 9) motif (Westin et al., (1998) Nature 395:199-202) of the nuclear receptor co-activator SRC-1. These structures indicate that the LXR binding pocket is large enough to accommodate the oxysterols, and that the LXR protein is flexible enough to accommodate changes in ligand shape. As has been observed in other steroid receptor structures (Brzozowski et al., (1997) Nature 389:753-758; Williams & Sigler, (1998) Nature 393:392-396; Matias et al., (2000) J. Biol. Chem. 275:26164-2617) a relatively small number of polar interactions are responsible for determining receptor specificity, with the tight binding a result of the ligand's close fit within the hydrophobic pocket. The structure of LXRβ with EPC shows some interesting features that can be explored to fully understand the binding and activation effect. These features might be relevant for drug discovery efforts, as binding of oxysterols to LXR does not appear to be sufficient for activation. The LXRβ/T317 structure shows markedly different interactions from those with EPC, indicating that potency does not require the molecule to fill the binding pocket.

### Laboratory Examples

The following Laboratory Examples have been included to illustrate preferred modes of the invention. Certain aspects of the following Laboratory Examples are described in terms of techniques and procedures found or contemplated by the present inventors to work well in the practice of the invention. These Laboratory Examples are exemplified through the use of standard laboratory practices of the inventors. In light of the present disclosure and the general level of skill in the art, those of skill will appreciate that the following Laboratory Examples are intended to be exemplary only and that numerous changes, modifications and alterations can be employed without departing from the spirit and scope of the invention.

### Laboratory Example 1

### LXR Expression and Purification

A minimal construct of the human LXRβ LBD (ligand binding domain) containing amino acids 214-462 and a thrombin cleavable his-tag was subcloned into the E. coli expression vector pRSETa (Invitrogen, Carlsbad, Calif.). After transforming into BL21(DE3) E. coli positive transformants were selected overnight at 37° C. on a LB plate containing 100 μg/ml carbenicilin. 3-4 colonies were selected and grown at 25° C. in 30 ml LB with 100 μg/ml carbenicilin, then used to inoculate 12×2L shaker flasks containing 1 liter of Terrific Broth II (Bio 101, Inc., Vista, Calif.) with 100 μg/ml carbenicilin at 2 ml/liter. Cultures were grown overnight at 22° C. without induction and harvested by centrifugation at 5000G for 10 min. Cell pellets were stored frozen at −80° C.

The original 12 L shaker flask purification was subsequently scaled up for fermenter growths. All steps were performed at 4° C. Cells were resuspended by adding 4× volume of NiA (25 mM imidazole pH 8.0, 150 mM NaCl, 3% 1,2 propane diol), then lysed by three passages through a cell homogenizer (APV Rannie, Copenhagen, Denmark), followed by centrifugation for 1 hour at 40,000 RPM in a Sorvall A641 to clarify the resultant solution.

The soluble protein was first purified with a 50 ml Ni⁺²-NTA affinity column (Qiagen, Valencia, Calif.), which was scaled up to a 150 ml column for the fermenter cultures. Cell lysate was loaded on to the pre-equilibrated column, followed by a 3-column wash with NiA, a 2-column wash with NiA with 50 mM imidazole, and eluted with a 10 column volume 50-500 mM imidazole gradient. LXR eluted at ˜300 mM imidazole. At this point, peak fractions were pooled and dialyzed against 10 mM Tris pH=8.0, 150 mM NaCl, 0.1 mM EDTA, 5% glycerol, separated into aliquots containing 30 mg of protein each, and stored frozen at −80° C.

For structural studies of protein compound complexes, protein was thawed and further purified using anion exchange with a 5 ml HiTrap HPQ column (AP Biosystems). After adding the relevant compound, his-tagged LXR was digested overnight at 4° C. with thrombin at a mass ration of 1:500. The digested protein was diluted to 25 mM salt with QO buffer (10 mM Tris pH 8.0, 0.1 mM EDTA, 5% glycerol, 5 mM DTT), and then loaded on to the pre-equilibrated anion exchange column. The column was washed with 3 columns of QO plus 30 mM NaCl followed by a 20-column 0-250 mM NaCl gradient. Complexed LXR usually eluted as a sharp peak at ˜150 mM NaCl. The eluted protein was dialyzed against 10 mM Tris pH 8.0, 0.1 mM EDTA, 5% glycerol, 5 mM DTT, 150 mM NaCl; additional compound was added, and the protein was concentrated to 12-14 mg/ml for crystallization trials.

### Laboratory Example 2

### Crystallization of a LXR Ligand Binding Domain

Crystallization trials were carried out using the hanging drop method by mixing 2 μl of protein solution with 2 μl of well buffer. Both the LXRβ/ligand complexes crystallized out of 10-12% PEG 3350-8000 with 0.2M salt at 40° C. Usable LXRβ/T0901317 crystals were obtained from a number of salts including NaF, KF, NaCl, KCl, Na formate, Na acetate, and K acetate. The best LXRβ/EPC (24(S), 25-epoxycholesterol) crystals were obtained with ammonium sulfate and lithium sulfate salts. All crystals took 4-6 weeks to grow. Crystals were frozen in LN₂ after transferring stepwise to a cryo buffer containing the well buffer with 30% PEG 400.

### Laboratory Example 3

### Structure Determination

All data was obtained at the IMCA CAT 17 ID beam line at the Advanced Photon Source (APS) at a wavelength of 1 Å on a MAR CCD detector. Crystals of the LXRβ/T0901317 complex diffracted to 2.3 A, and were in the space group P2₁2₁2₁ with unit cell parameters a=60.25 b=82.454, c=123.175. Crystals of the LXRβ/24,25 epoxycholesterol/SRC1 complex diffracted to 2.8 Å, and were in the space group C²²²₁ with unit cell parameters a=71.17 b=120.01, c=147.56. All diffraction data was integrated and scaled using HKL2000 (Otwinowski & Minor, (1997) Method Enzymol. 276: 307-326). The LXRβ/T0901317 structure was solved by molecular replacement using the program AMoRe with a truncated monomeric form of human RARγ as a search model (Table 5). The LXRβ/EPC/SRC1 structure was solved by molecular replacement using the program AMoRe and a refined LXR A subunit from the LXRβ/T0901317 structure as a search model (Table 3).

In both complex structures there were two molecules in the asymmetric unit related by a non-crystallographic dyad. In the case of the T317 complex, the two subunits were substantially different, so non-crystallographic averaging could not be utilized. Structures were subjected to multiple rounds of building using QUANTAT™ software (Molecular Simulations Inc., San Diego, Calif.) and refined using CNX software (Molecular Simulations Inc., San Diego, Calif.).

### Laboratory Example 4

### Mutant Construction and Transactivation Measurements by Transient Transfection

4.1 Plasmid Construction

Wild type and mutant LXR transactivation activity was measured in CV-1 cells. Gal4 expression constructs were prepared using the wild type or mutated ligand binding domains (LBDs) of human LXRα and LXRβ (Lehmann et al., (1997) J. Biol. Chem. 272:3137-3140). Wild type LBDs for LXRα (amino acids 164-447) and LXRβ (amino acids 155-462) were subcloned into the pSG-5 Gal4 vector as a fusion with the Gal4 DNA binding domain as KpnI-BamHI fragments. Single amino acid changes in the wild-type receptor LBD proteins were made by utilizing a two-step PCR technique with the corresponding KpnI and BamHI restriction cut sites incorporated at the 5′ and 3′ ends of the DNA fragment. The mutant DNA fragment was subcloned back in to the original pSG5-Gal4 parental vector.

A human SRC-1 construct (representing amino acids 1-1005) (Onate et al., (1995) Science 270:1354-1357) was prepared in the pSG5 expression vector (Stratagene Corp., La Jolla, Calif.). The reporter plasmid UAS-tk-SPAP was generated by insertion of five copies of a Gal4 DNA-binding element into pGl2-tk-SPAP. The β-galactosidase expression plasmid pCH110 (Pharmacia, Piscataway, N.J.) was employed as an internal control for transfection efficiency.

4.2 Transfection and Drug Delivery

Transfection mixes contained a total of 80 ng total DNA (2 ng receptor plasmid, 8 ng UAS-tk-SPAP reporter plasmid, 7 ng SRC-1 expression plasmid, 25 ng pCH110, and 38 ng of carrier plasmid (pBluescript, Stratagene, La Jolla, Calif.) and 0.7 μl Lipofectamine (Life Technologies, Inc., Rockville, Md.) were added to each well. After 6 to 18 hours, compounds were added at a concentration of 10 μM and incubated for 18 hours prior to determination of reporter activity.

4.3 Reporter Assays

Alkaline phosphatase activity was assayed by adding 200 μl substrate (7 mM p-nitrophenyl phosphate in 1 M diethanolamine/0.28 M sodium chloride/0.5 mM magnesium chloride, pH 9.85) to each well of the plates, incubating at room temperature, and reading absorbance at 405 nm.

β-Galactosidase normalization was performed by adding 200 μl of substrate (1.1 mg/ml o-nitrophenyl β-D-galactopyranoside, 0.13% Triton X-100 in 0.1 M sodium phosphate buffer, pH 7.2) to cell plate containing 60 μl of medium, incubating at room temperature, and reading absorbance at 405 nm.

### REFERENCES

The references listed below as well as all references cited in the specification are incorporated herein by reference to the extent that they supplement, explain, provide a background for or teach methodology, techniques and/or compositions employed herein.

Altschul et al., (1990) J. Mol. Biol. 215: 403-10

Ausubel et al., (1989) Current Protocols in Molecular Biology, Greene Publishing Associates and Wiley Interscience, New York

Bartlett et al., (1989) Special Pub., Royal Chem. Soc. 78: 182-96

Blundell & Johnson (1985) Method. Enzymol. 114A & 115B

Blundell et al., (1976) Protein Crystallography, Academic Press

Bohm, (1992) J. Comput. Aid. Mol. Des. 6: 61-78

Brooks et al., (1983) J. Comp. Chem., 8: 132

Brünger, (1992) X-PLOR, Version 3.1. A System for X-ray Crystallography and NMR, Yale University Press, New Haven, Conn.

Brzozowski et al., (1997) Nature 389:753-758

Case et al., (1997) AMBER 5, University of California, San Francisco

Cohen et al., (1990) J. Med. Chem. 33: 883-94

Creighton, (1983) Proteins: Structures and Molecular Principles, W.H. Freeman & Co., New York

Drewes et al., (1996) Mol. Cell. Biol. 16:925-31

Ducruix & Geige, (1992) Crystallization of Nucleic Acids and Proteins: A Practical Approach, IRL Press, Oxford, England

Eisen et al., (1994). Proteins 19: 199-221

Forman et al., (1997) Proc. Nat Acad. Sci. U.S.A. 94:10588-10593

Goodford, (1985) J. Med. Chem. 28: 849-57

Goodsell & Olsen, (1990) Proteins 8: 195-202

Gribskov et al., (1986) Nucl. Acids. Res. 14: 6745

Hauptman, (1997) Curr. Opin. Struct. Biol. 7: 672-80

Janowski et al., (1999) Proc. Nat. Acad. Sci. U.S.A. 96:266-271

Kuntz et al., (1992) J. Mol. Biol. 161: 269-88

Kyte & Doolittle, (1982), J. Mol. Biol. 157: 105-132

Lambert, (1997) in Practical Application of Computer-Aided Drug Design, (Charifson, ed.) Marcel-Dekker, New York, pp.243-303

Lattman, (1985) Method Enzymol. 115: 55-77

Lehmann et al., (1997) J. Biol. Chem. 272(6): 3137-3140

Makishima et al., (1999) Science 284:1362-1365

Martin, (1992) J. Med. Chem. 35: 2145-54

Matias et al., (2000) J. Biol. Chem. 275:26164-2617

McPherson, (1982) Preparation and Analysis of Protein Crystals, John Wiley, New York

McPherson, (1990) Eur. J. Biochem. 189:1-23

McRee, (1992) J. Mol. Graphics 10: 44-47

Miranker & Karplus, (1991) Proteins 11: 29-34

Navaza & Saludjian, (1997) Method Enzymol. 276A: 581-94

Navia & Murcko, (1992) Curr. Opin. Struc. Biol. 2: 202-10

Needleman et al., (1970) J. Mol. Biol. 48: 443

Nelson et al., (1981) J. Biol. Chem. 256:1067-1068

Nicholls et al., (1991) Proteins 11: 281-96

Nishibata & Itai, (1991) Tetrahedron 47: 8985

Onate et al., (1995) Science 270:1354-1357

Otwinowski & Minor, (1997) Method Enzymol. 276: 307-326

Otwinowski, (1991) in Isomorphous Replacement and Anomalous Scattering, (Evans & Leslie, eds.), 80-86, Daresbury Laboratory, Daresbury, United Kingdom

Parks et al., (1999) Science 284:1365-1368

Pearlman et al., (1995) Comput. Phys. Commun. 91: 1-41

Rarey et al., (1996) J. Comput. Aid. Mol. Des. 10:41-54

Renaud et al., (1995) Nature 378:681-689

Rossmann (ed), (1972) The Molecular Replacement Method, Gordon & Breach, New York

Sambrook et al., (1989) Molecular Cloning: A Laboratory Manual, Cold Spring Harbor Laboratory, New York,

Saucier et al., (1985) J. Biol. Chem. 260: 14571-14579

Schultz et al., (2000) Gene Dev. 14: 2831-2838

Schwartz et al. (eds.), (1979), Atlas of Protein Sequence and Structure, National Biomedical Research Foundation, pp. 357-358

Sheldrick (1990) Acta Cryst. A 46: 467

Sladek et al., Genes Dev. 4:2353-65

Smith et al., (1981) Adv. Appl. Math. 2:482

Spencer et al., (2001) J. Med. Chem. 44: 886-897

Van Holde, (1971) Physical Biochemistry, Prentice-Hall, New Jersey, pp.221-37

Venkateswaran et al., (2000) Proc. Nat. Acad. Sci. U.S.A. 97:12097-12102

Weber, (1991) Adv. Protein Chem. 41:1-36

Weeks et al., (1993) Acta Cryst. D 49: 179

Wellner, (1971) Anal. Chem. 43:597

Westin et al., (1998) Nature 395:199-202

Wetmur & Davidson, (1968) J. Mol. Biol. 31: 349-70

Williams & Sigler, (1998) Nature 393:392-396

U.S. Pat. No. 4,554,101

U.S. Pat. No. 5,463,564

U.S. Pat. No. 5,834,228

U.S. Pat. No. 5,872,011

U.S. Pat. No. 6,008,033

WO00/025134

WO 00/037077

WO 84/03564

TABLE 1
CRYSTALLOGRAPHIC DATA AND REFINEMENT

| Crystal | T317 Complex | | EPC complex | |
|---|---|---|---|---|
| Space Group | P2₁2₁2₁ | | C222₁ | |
| Unit Cell | a = 60.25, | b = 82.45 , | a = 71.17 | b = 120.01, |
| | c = 123.18 | | c = 147.56 | |
| | α= β= γ= 90° | | α= β= γ = 90° | |
| Resolution Range | 30.-2.3 | | 20-2.8 | |
| Observations (Unique) | 308126 (27968) | | 126333 (15680) | |
| Completeness | 95.7 (98.7) | | 98.3 (99.3) | |
| I/σ | 29.8 (3.4) | | 11.6 (3.1) | |
| Rmerge % | 5.2 (29) | | 5.5 (35) | |
| Refinement Statistics | | | | |
| Resolution Range | 30.-2.3 | | 20-2.8 | |
| % R_{free} | 7 | | 7 | |
| R_{cryst} R_{free} | 22.1 (26.2) | | 21.6 (27.5) | |
| Protein atoms | 3724 | | 4124 | |
| Ligand atoms | 93 | | 58 | |
| Water Molecules | 334 | | 101 | |
| RMSD bonds/angles | 0.0085/1.675 | | 0.0171/1.807 | |

TABLE 2
ATOMIC COORDINATE DATA FOR CRYSTALLIZED HUMAN LXR
IN COMPLEX WITH SRC1 and EPC

| | ATOM | | | | | | | | |
|---|---|---|---|---|---|---|---|---|---|
| ATOM | TYPE | RESIDUE | PROTEIN # | # | X | Y | Z | OCC | B |
| 1 | CB | ALA | A | 218 | 28.344 | 16.106 | 96.698 | 1.00 | 55.76 |
| 2 | C | ALA | A | 218 | 29.653 | 18.044 | 95.802 | 1.00 | 55.24 |
| 3 | O | ALA | A | 218 | 30.106 | 18.364 | 94.699 | 1.00 | 54.28 |
| 4 | N | ALA | A | 218 | 27.664 | 17.216 | 94.572 | 1.00 | 64.79 |
| 5 | CA | ALA | A | 218 | 28.265 | 17.434 | 95.930 | 1.00 | 58.73 |
| 6 | N | ALA | A | 219 | 30.314 | 18.224 | 96.938 | 1.00 | 52.70 |
| 7 | CA | ALA | A | 219 | 31.664 | 18.775 | 96.947 | 1.00 | 51.55 |
| 8 | CB | ALA | A | 219 | 31.632 | 20.293 | 97.099 | 1.00 | 46.87 |
| 9 | C | ALA | A | 219 | 32.490 | 18.142 | 98.068 | 1.00 | 51.86 |
| 10 | O | ALA | A | 219 | 31.990 | 17.352 | 98.874 | 1.00 | 55.04 |
| 11 | N | LEU | A | 220 | 33.768 | 18.475 | 98.111 | 1.00 | 51.33 |
| 12 | CA | LEU | A | 220 | 34.644 | 17.936 | 99.143 | 1.00 | 45.85 |
| 13 | CB | LEU | A | 220 | 36.098 | 18.230 | 98.786 | 1.00 | 42.81 |
| 14 | CG | LEU | A | 220 | 36.953 | 16.990 | 98.618 | 1.00 | 45.64 |
| 15 | CD1 | LEU | A | 220 | 36.552 | 16.276 | 97.358 | 1.00 | 47.27 |
| 16 | CD2 | LEU | A | 220 | 38.412 | 17.371 | 98.575 | 1.00 | 40.99 |
| 17 | C | LEU | A | 220 | 34.316 | 18.571 | 100.497 | 1.00 | 43.50 |
| 18 | O | LEU | A | 220 | 34.040 | 19.755 | 100.599 | 1.00 | 47.45 |
| 19 | N | THR | A | 221 | 34.328 | 17.777 | 101.546 | 1.00 | 41.43 |
| 20 | CA | THR | A | 221 | 34.062 | 18.325 | 102.870 | 1.00 | 39.33 |
| 21 | CB | THR | A | 221 | 33.764 | 17.233 | 103.878 | 1.00 | 38.56 |
| 22 | OG1 | THR | A | 221 | 34.797 | 16.239 | 103.820 | 1.00 | 37.25 |
| 23 | CG2 | THR | A | 221 | 32.401 | 16.603 | 103.572 | 1.00 | 15.24 |
| 24 | C | THR | A | 221 | 35.317 | 19.037 | 103.331 | 1.00 | 39.68 |
| 25 | O | THR | A | 221 | 36.431 | 18.689 | 102.928 | 1.00 | 45.38 |
| 26 | N | ALA | A | 222 | 35.152 | 20.045 | 104.166 | 1.00 | 37.55 |
| 27 | CA | ALA | A | 222 | 36.325 | 20.774 | 104.604 | 1.00 | 37.06 |
| 28 | CB | ALA | A | 222 | 35.912 | 21.793 | 105.621 | 1.00 | 38.76 |
| 29 | C | ALA | A | 222 | 37.331 | 19.767 | 105.193 | 1.00 | 32.94 |
| 30 | O | ALA | A | 222 | 38.550 | 19.898 | 105.059 | 1.00 | 28.56 |
| 31 | N | ALA | A | 223 | 36.784 | 18.726 | 105.802 | 1.00 | 27.58 |
| 32 | CA | ALA | A | 223 | 37.588 | 17.699 | 106.412 | 1.00 | 23.17 |
| 33 | CB | ALA | A | 223 | 36.696 | 16.774 | 107.142 | 1.00 | 9.46 |
| 34 | C | ALA | A | 223 | 38.372 | 16.964 | 105.339 | 1.00 | 25.13 |
| 35 | O | ALA | A | 223 | 39.596 | 17.006 | 105.347 | 1.00 | 29.33 |
| 36 | N | GLN | A | 224 | 37.662 | 16.300 | 104.426 | 1.00 | 24.64 |
| 37 | CA | GLN | A | 224 | 38.289 | 15.570 | 103.341 | 1.00 | 30.36 |
| 38 | CB | GLN | A | 224 | 37.234 | 15.098 | 102.343 | 1.00 | 22.67 |
| 39 | CG | GLN | A | 224 | 36.338 | 14.029 | 102.858 | 1.00 | 32.52 |
| 40 | CD | GLN | A | 224 | 35.492 | 13.449 | 101.760 | 1.00 | 36.79 |
| 41 | OE1 | GLN | A | 224 | 34.638 | 14.145 | 101.183 | 1.00 | 33.52 |
| 42 | NE2 | GLN | A | 224 | 35.726 | 12.171 | 101.447 | 1.00 | 46.08 |
| 43 | C | GLN | A | 224 | 39.342 | 16.409 | 102.589 | 1.00 | 34.36 |
| 44 | O | GLN | A | 224 | 40.468 | 15.970 | 102.294 | 1.00 | 39.02 |
| 45 | N | GLU | A | 225 | 38.968 | 17.618 | 102.238 | 1.00 | 32.23 |
| 46 | CA | GLU | A | 225 | 39.909 | 18.444 | 101.544 | 1.00 | 31.48 |
| 47 | CB | GLU | A | 225 | 39.333 | 19.833 | 101.384 | 1.00 | 42.03 |
| 48 | CG | GLU | A | 225 | 39.876 | 20.576 | 100.206 | 1.00 | 52.07 |
| 49 | CD | GLU | A | 225 | 38.993 | 21.761 | 99.842 | 1.00 | 69.24 |
| 50 | OE1 | GLU | A | 225 | 37.812 | 21.804 | 100.288 | 1.00 | 68.23 |
| 51 | OE2 | GLU | A | 225 | 39.477 | 22.643 | 99.098 | 1.00 | 72.72 |
| 52 | C | GLU | A | 225 | 41.187 | 18.495 | 102.360 | 1.00 | 27.39 |
| 53 | O | GLU | A | 225 | 42.255 | 18.255 | 101.831 | 1.00 | 29.04 |
| 54 | N | LEU | A | 226 | 41.053 | 18.777 | 103.656 | 1.00 | 24.43 |
| 55 | CA | LEU | A | 226 | 42.187 | 18.897 | 104.560 | 1.00 | 20.16 |
| 56 | CB | LEU | A | 226 | 41.743 | 19.318 | 105.966 | 1.00 | 19.25 |
| 57 | CG | LEU | A | 226 | 42.892 | 19.672 | 106.914 | 1.00 | 15.29 |
| 58 | CD1 | LEU | A | 226 | 43.785 | 20.719 | 106.248 | 1.00 | 20.10 |
| 59 | CD2 | LEU | A | 226 | 42.349 | 20.229 | 108.205 | 1.00 | 25.61 |
| 60 | C | LEU | A | 226 | 42.939 | 17.605 | 104.640 | 1.00 | 20.54 |
| 61 | O | LEU | A | 226 | 44.159 | 17.598 | 104.723 | 1.00 | 21.73 |
| 62 | N | MET | A | 227 | 42.216 | 16.498 | 104.619 | 1.00 | 18.60 |
| 63 | CA | MET | A | 227 | 42.866 | 15.199 | 104.633 | 1.00 | 16.23 |
| 64 | CB | MET | A | 227 | 41.798 | 14.120 | 104.774 | 1.00 | 9.90 |
| 65 | CG | MET | A | 227 | 42.353 | 12.738 | 104.934 | 1.00 | 22.30 |
| 66 | SD | MET | A | 227 | 42.829 | 12.043 | 103.385 | 1.00 | 30.16 |
| 67 | CE | MET | A | 227 | 41.187 | 11.531 | 102.760 | 1.00 | 36.03 |
| 68 | C | MET | A | 227 | 43.659 | 15.005 | 103.291 | 1.00 | 20.80 |
| 69 | O | MET | A | 227 | 44.838 | 14.659 | 103.298 | 1.00 | 18.00 |
| 70 | N | ILE | A | 228 | 43.006 | 15.230 | 102.143 | 1.00 | 21.23 |
| 71 | CA | ILE | A | 228 | 43.658 | 15.076 | 100.846 | 1.00 | 15.74 |
| 72 | CB | ILE | A | 228 | 42.682 | 15.383 | 99.705 | 1.00 | 18.87 |
| 73 | CG2 | ILE | A | 228 | 43.387 | 15.298 | 98.377 | 1.00 | 5.25 |
| 74 | CG1 | ILE | A | 228 | 41.542 | 14.363 | 99.720 | 1.00 | 12.45 |
| 75 | CD1 | ILE | A | 228 | 40.681 | 14.448 | 98.519 | 1.00 | 6.20 |
| 76 | C | ILE | A | 228 | 44.895 | 15.947 | 100.733 | 1.00 | 15.29 |
| 77 | O | ILE | A | 228 | 45.887 | 15.530 | 100.143 | 1.00 | 11.10 |
| 78 | N | GLN | A | 229 | 44.838 | 17.164 | 101.276 | 1.00 | 16.77 |
| 79 | CA | GLN | A | 229 | 46.021 | 18.028 | 101.289 | 1.00 | 20.67 |
| 80 | CB | GLN | A | 229 | 45.658 | 19.408 | 101.773 | 1.00 | 16.16 |
| 81 | CG | GLN | A | 229 | 45.235 | 20.307 | 100.632 | 1.00 | 34.86 |
| 82 | CD | GLN | A | 229 | 44.291 | 21.436 | 101.072 | 1.00 | 46.19 |
| 83 | OE1 | GLN | A | 229 | 44.482 | 22.067 | 102.122 | 1.00 | 34.04 |
| 84 | NE2 | GLN | A | 229 | 43.261 | 21.689 | 100.261 | 1.00 | 54.62 |
| 85 | C | GLN | A | 229 | 47.082 | 17.389 | 102.215 | 1.00 | 24.70 |
| 86 | O | GLN | A | 229 | 48.253 | 17.317 | 101.879 | 1.00 | 29.53 |
| 87 | N | GLN | A | 230 | 46.649 | 16.908 | 103.373 | 1.00 | 27.75 |
| 88 | CA | GLN | A | 230 | 47.488 | 16.213 | 104.345 | 1.00 | 23.86 |
| 89 | CB | GLN | A | 230 | 46.555 | 15.463 | 105.299 | 1.00 | 40.92 |
| 90 | CG | GLN | A | 230 | 47.000 | 15.206 | 106.716 | 1.00 | 55.66 |
| 91 | CD | GLN | A | 230 | 45.786 | 15.006 | 107.676 | 1.00 | 58.41 |
| 92 | OE1 | GLN | A | 230 | 44.927 | 15.880 | 107.803 | 1.00 | 52.62 |
| 93 | NE2 | GLN | A | 230 | 45.731 | 13.865 | 108.341 | 1.00 | 62.25 |
| 94 | C | GLN | A | 230 | 48.313 | 15.171 | 103.567 | 1.00 | 25.51 |
| 95 | O | GLN | A | 230 | 49.552 | 15.245 | 103.525 | 1.00 | 24.06 |
| 96 | N | LEU | A | 231 | 47.625 | 14.202 | 102.943 | 1.00 | 21.46 |
| 97 | CA | LEU | A | 231 | 48.301 | 13.139 | 102.232 | 1.00 | 15.10 |
| 98 | CB | LEU | A | 231 | 47.308 | 12.176 | 101.635 | 1.00 | 16.30 |
| 99 | CG | LEU | A | 231 | 46.835 | 11.054 | 102.539 | 1.00 | 14.70 |
| 100 | CD1 | LEU | A | 231 | 46.032 | 10.109 | 101.662 | 1.00 | 8.73 |
| 101 | CD2 | LEU | A | 231 | 48.022 | 10.358 | 103.175 | 1.00 | 5.25 |
| 102 | C | LEU | A | 231 | 49.236 | 13.598 | 101.153 | 1.00 | 17.13 |
| 103 | O | LEU | A | 231 | 50.390 | 13.147 | 101.103 | 1.00 | 15.46 |
| 104 | N | VAL | A | 232 | 48.762 | 14.491 | 100.281 | 1.00 | 16.95 |
| 105 | CA | VAL | A | 232 | 49.618 | 14.966 | 99.196 | 1.00 | 15.39 |
| 106 | CB | VAL | A | 232 | 48.910 | 15.936 | 98.252 | 1.00 | 14.04 |
| 107 | CG1 | VAL | A | 232 | 49.911 | 16.605 | 97.355 | 1.00 | 5.25 |
| 108 | CG2 | VAL | A | 232 | 47.911 | 15.167 | 97.411 | 1.00 | 11.19 |
| 109 | C | VAL | A | 232 | 50.856 | 15.624 | 99.728 | 1.00 | 17.28 |
| 110 | O | VAL | A | 232 | 51.942 | 15.446 | 99.155 | 1.00 | 19.63 |
| 111 | N | ALA | A | 233 | 50.711 | 16.371 | 100.824 | 1.00 | 15.96 |
| 112 | CA | ALA | A | 233 | 51.888 | 17.022 | 101.431 | 1.00 | 18.52 |
| 113 | CB | ALA | A | 233 | 51.455 | 18.052 | 102.445 | 1.00 | 8.26 |
| 114 | C | ALA | A | 233 | 52.835 | 15.979 | 102.086 | 1.00 | 19.61 |
| 115 | O | ALA | A | 233 | 54.056 | 15.958 | 101.822 | 1.00 | 16.98 |
| 116 | N | ALA | A | 234 | 52.281 | 15.112 | 102.935 | 1.00 | 25.82 |
| 117 | CA | ALA | A | 234 | 53.113 | 14.071 | 103.562 | 1.00 | 27.97 |
| 118 | CB | ALA | A | 234 | 52.231 | 13.130 | 104.444 | 1.00 | 20.72 |
| 119 | C | ALA | A | 234 | 53.870 | 13.249 | 102.486 | 1.00 | 25.67 |
| 120 | O | ALA | A | 234 | 55.023 | 12.833 | 102.703 | 1.00 | 24.36 |
| 121 | N | GLN | A | 235 | 53.214 | 13.017 | 101.341 | 1.00 | 22.92 |
| 122 | CA | GLN | A | 235 | 53.837 | 12.259 | 100.257 | 1.00 | 25.97 |
| 123 | CB | GLN | A | 235 | 52.802 | 11.886 | 99.207 | 1.00 | 21.78 |
| 124 | CG | GLN | A | 235 | 53.336 | 11.420 | 97.871 | 1.00 | 28.41 |
| 125 | CD | GLN | A | 235 | 52.180 | 10.969 | 96.901 | 1.00 | 47.75 |
| 126 | OE1 | GLN | A | 235 | 51.692 | 9.827 | 96.976 | 1.00 | 41.32 |
| 127 | NE2 | GLN | A | 235 | 51.735 | 11.883 | 96.010 | 1.00 | 47.47 |
| 128 | C | GLN | A | 235 | 54.983 | 13.028 | 99.629 | 1.00 | 25.13 |
| 129 | O | GLN | A | 235 | 55.934 | 12.453 | 99.163 | 1.00 | 29.39 |
| 130 | N | LEU | A | 236 | 54.893 | 14.343 | 99.657 | 1.00 | 24.34 |
| 131 | CA | LEU | A | 236 | 55.930 | 15.200 | 99.122 | 1.00 | 20.09 |
| 132 | CB | LEU | A | 236 | 55.352 | 16.601 | 98.944 | 1.00 | 10.48 |
| 133 | CG | LEU | A | 236 | 56.263 | 17.528 | 98.144 | 1.00 | 13.40 |
| 134 | CD1 | LEU | A | 236 | 56.555 | 16.915 | 96.746 | 1.00 | 5.25 |
| 135 | CD2 | LEU | A | 236 | 55.580 | 18.860 | 98.025 | 1.00 | 6.03 |
| 136 | C | LEU | A | 236 | 57.137 | 15.249 | 100.103 | 1.00 | 19.25 |
| 137 | O | LEU | A | 236 | 58.314 | 15.081 | 99.728 | 1.00 | 14.99 |
| 138 | N | GLN | A | 237 | 56.842 | 15.478 | 101.370 | 1.00 | 17.73 |
| 139 | CA | GLN | A | 237 | 57.912 | 15.546 | 102.341 | 1.00 | 22.68 |
| 140 | CB | GLN | A | 237 | 57.343 | 16.022 | 103.675 | 1.00 | 26.67 |
| 141 | CG | GLN | A | 237 | 57.937 | 17.332 | 104.108 | 1.00 | 47.77 |
| 142 | CD | GLN | A | 237 | 59.451 | 17.329 | 103.931 | 1.00 | 59.33 |
| 143 | OE1 | GLN | A | 237 | 60.158 | 16.496 | 104.516 | 1.00 | 65.55 |
| 144 | NE2 | GLN | A | 237 | 59.953 | 18.246 | 103.110 | 1.00 | 63.55 |
| 145 | C | GLN | A | 237 | 58.711 | 14.241 | 102.527 | 1.00 | 18.77 |
| 146 | O | GLN | A | 237 | 59.930 | 14.258 | 102.665 | 1.00 | 12.71 |
| 147 | N | CYS | A | 238 | 58.019 | 13.107 | 102.503 | 1.00 | 18.52 |
| 148 | CA | CYS | A | 238 | 58.692 | 11.837 | 102.720 | 1.00 | 18.81 |
| 149 | CB | CYS | A | 238 | 57.679 | 10.681 | 102.932 | 1.00 | 10.34 |
| 150 | SG | CYS | A | 238 | 56.626 | 10.868 | 104.440 | 1.00 | 14.23 |
| 151 | C | CYS | A | 238 | 59.584 | 11.589 | 101.537 | 1.00 | 15.55 |
| 152 | O | CYS | A | 238 | 60.658 | 11.034 | 101.689 | 1.00 | 14.18 |
| 153 | N | ASN | A | 239 | 59.144 | 12.032 | 100.362 | 1.00 | 16.15 |
| 154 | CA | ASN | A | 239 | 59.940 | 11.852 | 99.160 | 1.00 | 18.70 |
| 155 | CB | ASN | A | 239 | 59.146 | 12.265 | 97.909 | 1.00 | 15.21 |
| 156 | CG | ASN | A | 239 | 59.912 | 11.937 | 96.576 | 1.00 | 26.14 |
| 157 | OD1 | ASN | A | 239 | 60.150 | 10.771 | 96.255 | 1.00 | 24.57 |
| 158 | ND2 | ASN | A | 239 | 60.306 | 12.973 | 95.825 | 1.00 | 12.25 |
| 159 | C | ASN | A | 239 | 61.237 | 12.692 | 99.281 | 1.00 | 21.54 |
| 160 | O | ASN | A | 239 | 62.351 | 12.205 | 99.044 | 1.00 | 20.85 |
| 161 | N | LYS | A | 240 | 61.098 | 13.954 | 99.671 | 1.00 | 25.84 |
| 162 | CA | LYS | A | 240 | 62.270 | 14.804 | 99.837 | 1.00 | 28.12 |
| 163 | CB | LYS | A | 240 | 61.852 | 16.198 | 100.326 | 1.00 | 27.47 |
| 164 | CG | LYS | A | 240 | 61.294 | 17.128 | 99.251 | 1.00 | 39.34 |
| 165 | CD | LYS | A | 240 | 61.175 | 18.563 | 99.770 | 1.00 | 42.46 |
| 166 | CE | LYS | A | 240 | 60.827 | 19.600 | 98.682 | 1.00 | 38.29 |
| 167 | NZ | LYS | A | 240 | 60.961 | 21.029 | 99.186 | 1.00 | 34.56 |
| 168 | C | LYS | A | 240 | 63.220 | 14.163 | 100.856 | 1.00 | 29.54 |
| 169 | O | LYS | A | 240 | 64.428 | 14.092 | 100.655 | 1.00 | 31.96 |
| 170 | N | ARG | A | 241 | 62.655 | 13.657 | 101.938 | 1.00 | 31.83 |
| 171 | CA | ARG | A | 241 | 63.464 | 13.066 | 102.972 | 1.00 | 36.01 |
| 172 | CB | ARG | A | 241 | 62.639 | 12.909 | 104.235 | 1.00 | 45.49 |
| 173 | CG | ARG | A | 241 | 63.457 | 13.137 | 105.514 | 1.00 | 61.86 |
| 174 | CD | ARG | A | 241 | 62.558 | 13.336 | 106.725 | 1.00 | 73.10 |
| 175 | NE | ARG | A | 241 | 61.443 | 12.384 | 106.713 | 1.00 | 80.71 |
| 176 | CZ | ARG | A | 241 | 60.173 | 12.713 | 106.481 | 1.00 | 79.67 |
| 177 | NH1 | ARG | A | 241 | 59.844 | 13.973 | 106.246 | 1.00 | 80.95 |
| 178 | NH2 | ARG | A | 241 | 59.228 | 11.780 | 106.485 | 1.00 | 81.60 |
| 179 | C | ARG | A | 241 | 64.143 | 11.746 | 102.643 | 1.00 | 33.51 |
| 180 | O | ARG | A | 241 | 64.879 | 11.214 | 103.447 | 1.00 | 35.57 |
| 181 | N | SER | A | 242 | 63.933 | 11.223 | 101.455 | 1.00 | 31.82 |
| 182 | CA | SER | A | 242 | 64.566 | 9.975 | 101.102 | 1.00 | 27.53 |
| 183 | CB | SER | A | 242 | 63.518 | 8.881 | 100.876 | 1.00 | 16.61 |
| 184 | OG | SER | A | 242 | 63.011 | 8.316 | 102.079 | 1.00 | 24.71 |
| 185 | C | SER | A | 242 | 65.397 | 10.141 | 99.835 | 1.00 | 31.35 |
| 186 | O | SER | A | 242 | 66.444 | 9.486 | 99.685 | 1.00 | 36.01 |
| 187 | N | PHE | A | 243 | 64.948 | 11.015 | 98.929 | 1.00 | 28.64 |
| 188 | CA | PHE | A | 243 | 65.646 | 11.191 | 97.679 | 1.00 | 28.31 |
| 189 | CB | PHE | A | 243 | 64.738 | 10.714 | 96.551 | 1.00 | 26.61 |
| 190 | CG | PHE | A | 243 | 64.560 | 9.208 | 96.505 | 1.00 | 19.50 |
| 191 | CD1 | PHE | A | 243 | 65.604 | 8.381 | 96.129 | 1.00 | 26.67 |
| 192 | CD2 | PHE | A | 243 | 63.368 | 8.621 | 96.873 | 1.00 | 26.29 |
| 193 | CE1 | PHE | A | 243 | 65.466 | 6.989 | 96.127 | 1.00 | 28.84 |
| 194 | CE2 | PHE | A | 243 | 63.220 | 7.219 | 96.872 | 1.00 | 25.73 |
| 195 | CZ | PHE | A | 243 | 64.276 | 6.409 | 96.495 | 1.00 | 22.93 |
| 196 | C | PHE | A | 243 | 66.217 | 12.572 | 97.378 | 1.00 | 33.43 |
| 197 | O | PHE | A | 243 | 66.665 | 12.840 | 96.274 | 1.00 | 33.13 |
| 198 | N | SER | A | 244 | 66.219 | 13.448 | 98.368 | 1.00 | 38.40 |
| 199 | CA | SER | A | 244 | 66.809 | 14.751 | 98.167 | 1.00 | 43.23 |
| 200 | CB | SER | A | 244 | 66.403 | 15.675 | 99.279 | 1.00 | 37.51 |
| 201 | OG | SER | A | 244 | 65.058 | 16.045 | 99.054 | 1.00 | 61.16 |
| 202 | C | SER | A | 244 | 68.316 | 14.621 | 98.159 | 1.00 | 47.85 |
| 203 | O | SER | A | 244 | 69.009 | 15.613 | 97.995 | 1.00 | 53.73 |
| 204 | N | ASP | A | 245 | 68.819 | 13.400 | 98.339 | 1.00 | 50.07 |
| 205 | CA | ASP | A | 245 | 70.258 | 13.149 | 98.349 | 1.00 | 49.87 |
| 206 | CB | ASP | A | 245 | 70.710 | 12.722 | 99.756 | 1.00 | 47.69 |
| 207 | CG | ASP | A | 245 | 70.531 | 13.813 | 100.766 | 1.00 | 47.80 |
| 208 | OD1 | ASP | A | 245 | 69.384 | 14.271 | 100.958 | 1.00 | 52.92 |
| 209 | OD2 | ASP | A | 245 | 71.538 | 14.224 | 101.360 | 1.00 | 48.83 |
| 210 | C | ASP | A | 245 | 70.603 | 12.074 | 97.328 | 1.00 | 50.73 |
| 211 | O | ASP | A | 245 | 70.188 | 10.939 | 97.492 | 1.00 | 54.30 |
| 212 | N | GLN | A | 246 | 71.345 | 12.438 | 96.285 | 1.00 | 49.92 |
| 213 | CA | GLN | A | 246 | 71.698 | 11.485 | 95.270 | 1.00 | 53.86 |
| 214 | CB | GLN | A | 246 | 72.615 | 12.156 | 94.241 | 1.00 | 56.38 |
| 215 | CG | GLN | A | 246 | 71.730 | 12.876 | 93.183 | 1.00 | 66.03 |
| 216 | CD | GLN | A | 246 | 72.205 | 14.274 | 92.816 | 1.00 | 65.94 |
| 217 | OE1 | GLN | A | 246 | 71.536 | 15.012 | 92.082 | 1.00 | 58.93 |
| 218 | NE2 | GLN | A | 246 | 73.366 | 14.644 | 93.328 | 1.00 | 69.59 |
| 219 | C | GLN | A | 246 | 72.298 | 10.287 | 95.978 | 1.00 | 52.91 |
| 220 | O | GLN | A | 246 | 73.502 | 10.241 | 96.261 | 1.00 | 58.02 |
| 221 | N | PRO | A | 247 | 71.436 | 9.294 | 96.264 | 1.00 | 49.01 |
| 222 | CD | PRO | A | 247 | 70.165 | 9.186 | 95.511 | 1.00 | 44.83 |
| 223 | CA | PRO | A | 247 | 71.769 | 8.053 | 96.969 | 1.00 | 46.95 |
| 224 | CB | PRO | A | 247 | 70.749 | 7.035 | 96.473 | 1.00 | 47.91 |
| 225 | CG | PRO | A | 247 | 69.908 | 7.738 | 95.465 | 1.00 | 47.27 |
| 226 | C | PRO | A | 247 | 73.168 | 7.540 | 96.878 | 1.00 | 49.19 |
| 227 | O | PRO | A | 247 | 73.844 | 7.750 | 95.886 | 1.00 | 46.72 |
| 228 | N | ALA | A | 248 | 73.576 | 6.865 | 97.951 | 1.00 | 52.55 |
| 229 | CA | ALA | A | 248 | 74.918 | 6.318 | 98.045 | 1.00 | 53.13 |
| 230 | CB | ALA | A | 248 | 75.411 | 6.320 | 99.511 | 1.00 | 58.29 |
| 231 | C | ALA | A | 248 | 74.928 | 4.917 | 97.499 | 1.00 | 49.47 |
| 232 | O | ALA | A | 248 | 75.440 | 4.005 | 98.150 | 1.00 | 55.81 |
| 233 | N | VAL | A | 249 | 74.408 | 4.759 | 96.290 | 1.00 | 41.18 |
| 234 | CA | VAL | A | 249 | 74.342 | 3.444 | 95.717 | 1.00 | 40.05 |
| 235 | CB | VAL | A | 249 | 72.916 | 3.094 | 95.363 | 1.00 | 39.10 |
| 236 | CG1 | VAL | A | 249 | 72.026 | 3.387 | 96.522 | 1.00 | 32.01 |
| 237 | CG2 | VAL | A | 249 | 72.482 | 3.872 | 94.129 | 1.00 | 38.08 |
| 238 | C | VAL | A | 249 | 75.212 | 3.223 | 94.495 | 1.00 | 40.64 |
| 239 | O | VAL | A | 249 | 75.456 | 4.147 | 93.722 | 1.00 | 41.34 |
| 240 | N | THR | A | 250 | 75.680 | 1.982 | 94.368 | 1.00 | 36.01 |
| 241 | CA | THR | A | 250 | 76.510 | 1.535 | 93.283 | 1.00 | 37.57 |
| 242 | CB | THR | A | 250 | 76.282 | 0.055 | 93.103 | 1.00 | 37.83 |
| 243 | OG1 | THR | A | 250 | 76.392 | −0.569 | 94.376 | 1.00 | 33.70 |
| 244 | CG2 | THR | A | 250 | 77.301 | −0.554 | 92.146 | 1.00 | 39.78 |
| 245 | C | THR | A | 250 | 76.104 | 2.262 | 92.013 | 1.00 | 41.06 |
| 246 | O | THR | A | 250 | 74.927 | 2.280 | 91.655 | 1.00 | 43.68 |
| 247 | N | PRO | A | 251 | 77.068 | 2.862 | 91.305 | 1.00 | 42.94 |
| 248 | CD | PRO | A | 251 | 78.386 | 3.265 | 91.818 | 1.00 | 44.90 |
| 249 | CA | PRO | A | 251 | 76.757 | 3.590 | 90.065 | 1.00 | 45.01 |
| 250 | CB | PRO | A | 251 | 78.025 | 4.414 | 89.831 | 1.00 | 40.94 |
| 251 | CG | PRO | A | 251 | 78.528 | 4.647 | 91.208 | 1.00 | 44.44 |
| 252 | C | PRO | A | 251 | 76.405 | 2.713 | 88.848 | 1.00 | 44.95 |
| 253 | O | PRO | A | 251 | 76.944 | 1.628 | 88.678 | 1.00 | 47.75 |
| 254 | N | TRP | A | 252 | 75.511 | 3.203 | 88.005 | 1.00 | 40.56 |
| 255 | CA | TRP | A | 252 | 75.147 | 2.469 | 86.826 | 1.00 | 41.27 |
| 256 | CB | TRP | A | 252 | 73.933 | 3.143 | 86.195 | 1.00 | 45.59 |
| 257 | CG | TRP | A | 252 | 73.292 | 2.423 | 85.043 | 1.00 | 42.31 |
| 258 | CD2 | TRP | A | 252 | 72.337 | 1.352 | 85.129 | 1.00 | 49.14 |
| 259 | CE2 | TRP | A | 252 | 71.892 | 1.079 | 83.824 | 1.00 | 49.10 |
| 260 | CE3 | TRP | A | 252 | 71.836 | 0.578 | 86.183 | 1.00 | 47.81 |
| 261 | CD1 | TRP | A | 252 | 73.379 | 2.753 | 83.738 | 1.00 | 36.71 |
| 262 | NE1 | TRP | A | 252 | 72.534 | 1.965 | 82.993 | 1.00 | 49.62 |
| 263 | CZ2 | TRP | A | 252 | 70.933 | 0.092 | 83.541 | 1.00 | 43.00 |
| 264 | CZ3 | TRP | A | 252 | 70.888 | −0.402 | 85.897 | 1.00 | 47.13 |
| 265 | CH2 | TRP | A | 252 | 70.461 | −0.643 | 84.585 | 1.00 | 34.10 |
| 266 | C | TRP | A | 252 | 76.336 | 2.474 | 85.859 | 1.00 | 42.16 |
| 267 | O | TRP | A | 252 | 76.880 | 3.528 | 85.521 | 1.00 | 39.92 |
| 268 | N | PRO | A | 253 | 76.775 | 1.288 | 85.428 | 1.00 | 44.33 |
| 269 | CD | PRO | A | 253 | 76.530 | −0.001 | 86.089 | 1.00 | 39.51 |
| 270 | CA | PRO | A | 253 | 77.905 | 1.217 | 84.493 | 1.00 | 50.55 |
| 271 | CB | PRO | A | 253 | 78.264 | −0.266 | 84.476 | 1.00 | 46.99 |
| 272 | CG | PRO | A | 253 | 77.769 | −0.777 | 85.744 | 1.00 | 44.13 |
| 273 | C | PRO | A | 253 | 77.552 | 1.725 | 83.089 | 1.00 | 54.78 |
| 274 | O | PRO | A | 253 | 76.562 | 2.427 | 82.902 | 1.00 | 54.13 |
| 275 | N | LEU | A | 254 | 78.364 | 1.327 | 82.108 | 1.00 | 61.04 |
| 276 | CA | LEU | A | 254 | 78.152 | 1.721 | 80.717 | 1.00 | 65.82 |
| 277 | CB | LEU | A | 254 | 78.874 | 3.041 | 80.447 | 1.00 | 64.47 |
| 278 | CG | LEU | A | 254 | 80.379 | 2.884 | 80.214 | 1.00 | 66.62 |
| 279 | CD1 | LEU | A | 254 | 80.602 | 2.624 | 78.743 | 1.00 | 61.12 |
| 280 | CD2 | LEU | A | 254 | 81.118 | 4.131 | 80.633 | 1.00 | 68.68 |
| 281 | C | LEU | A | 254 | 78.630 | 0.649 | 79.705 | 1.00 | 69.59 |
| 282 | O | LEU | A | 254 | 79.495 | −0.198 | 80.002 | 1.00 | 72.20 |
| 283 | N | ALA | A | 259 | 81.190 | −4.549 | 77.975 | 1.00 | 75.57 |
| 284 | CA | ALA | A | 259 | 80.352 | −4.942 | 79.099 | 1.00 | 80.27 |
| 285 | CB | ALA | A | 259 | 78.962 | −5.344 | 78.616 | 1.00 | 81.51 |
| 286 | C | ALA | A | 259 | 80.969 | −6.073 | 79.932 | 1.00 | 81.62 |
| 287 | O | ALA | A | 259 | 82.008 | −6.638 | 79.582 | 1.00 | 83.94 |
| 288 | N | SER | A | 260 | 80.305 | −6.447 | 81.017 | 1.00 | 82.06 |
| 289 | CA | SER | A | 260 | 80.905 | −7.445 | 81.859 | 1.00 | 81.46 |
| 290 | CB | SER | A | 260 | 82.196 | −6.850 | 82.398 | 1.00 | 84.53 |
| 291 | OG | SER | A | 260 | 82.176 | −5.432 | 82.266 | 1.00 | 83.98 |
| 292 | C | SER | A | 260 | 80.046 | −7.901 | 83.006 | 1.00 | 81.71 |
| 293 | O | SER | A | 260 | 78.920 | −7.458 | 83.166 | 1.00 | 84.27 |
| 294 | N | ARG | A | 261 | 80.617 | −8.782 | 83.816 | 1.00 | 82.01 |
| 295 | CA | ARG | A | 261 | 79.938 | −9.305 | 84.980 | 1.00 | 81.77 |
| 296 | CB | ARG | A | 261 | 80.493 | −10.693 | 85.311 | 1.00 | 82.72 |
| 297 | CG | ARG | A | 261 | 80.331 | −11.109 | 86.760 | 1.00 | 84.08 |
| 298 | CD | ARG | A | 261 | 81.677 | −11.377 | 87.407 | 1.00 | 84.50 |
| 299 | NE | ARG | A | 261 | 81.918 | −10.602 | 88.627 | 1.00 | 82.67 |
| 300 | CZ | ARG | A | 261 | 82.978 | −9.818 | 88.788 | 1.00 | 82.29 |
| 301 | NH1 | ARG | A | 261 | 83.856 | −9.715 | 87.813 | 1.00 | 81.99 |
| 302 | NH2 | ARG | A | 261 | 83.176 | −9.162 | 89.919 | 1.00 | 82.23 |
| 303 | C | ARG | A | 261 | 80.064 | −8.366 | 86.181 | 1.00 | 80.36 |
| 304 | O | ARG | A | 261 | 79.656 | −8.718 | 87.268 | 1.00 | 83.44 |
| 305 | N | ASP | A | 262 | 80.610 | −7.169 | 85.986 | 1.00 | 79.83 |
| 306 | CA | ASP | A | 262 | 80.751 | −6.212 | 87.090 | 1.00 | 79.82 |
| 307 | CB | ASP | A | 262 | 82.137 | −5.542 | 87.101 | 1.00 | 83.43 |
| 308 | CG | ASP | A | 262 | 82.926 | −5.858 | 88.369 | 1.00 | 84.30 |
| 309 | OD1 | ASP | A | 262 | 83.099 | −7.063 | 88.672 | 1.00 | 83.98 |
| 310 | OD2 | ASP | A | 262 | 83.376 | −4.908 | 89.056 | 1.00 | 83.90 |
| 311 | C | ASP | A | 262 | 79.695 | −5.151 | 86.972 | 1.00 | 79.08 |
| 312 | O | ASP | A | 262 | 79.499 | −4.362 | 87.878 | 1.00 | 82.58 |
| 313 | N | ALA | A | 263 | 79.017 | −5.140 | 85.835 | 1.00 | 77.24 |
| 314 | CA | ALA | A | 263 | 77.953 | −4.182 | 85.612 | 1.00 | 74.16 |
| 315 | CB | ALA | A | 263 | 78.086 | −3.558 | 84.236 | 1.00 | 75.62 |
| 316 | C | ALA | A | 263 | 76.657 | −4.981 | 85.728 | 1.00 | 71.38 |
| 317 | O | ALA | A | 263 | 75.633 | −4.485 | 86.230 | 1.00 | 70.95 |
| 318 | N | ARG | A | 264 | 76.718 | −6.239 | 85.300 | 1.00 | 65.44 |
| 319 | CA | ARG | A | 264 | 75.545 | −7.075 | 85.352 | 1.00 | 64.95 |
| 320 | CB | ARG | A | 264 | 75.782 | −8.370 | 84.582 | 1.00 | 68.61 |
| 321 | CG | ARG | A | 264 | 74.496 | −9.125 | 84.278 | 1.00 | 77.09 |
| 322 | CD | ARG | A | 264 | 74.746 | −10.578 | 84.014 | 1.00 | 83.87 |
| 323 | NE | ARG | A | 264 | 74.515 | −11.430 | 85.182 | 1.00 | 84.70 |
| 324 | CZ | ARG | A | 264 | 74.299 | −12.732 | 85.081 | 1.00 | 84.41 |
| 325 | NH1 | ARG | A | 264 | 74.286 | −13.282 | 83.883 | 1.00 | 83.98 |
| 326 | NH2 | ARG | A | 264 | 74.103 | −13.475 | 86.162 | 1.00 | 85.25 |
| 327 | C | ARG | A | 264 | 75.268 | −7.371 | 86.816 | 1.00 | 61.13 |
| 328 | O | ARG | A | 264 | 74.145 | −7.746 | 87.199 | 1.00 | 60.64 |
| 329 | N | GLN | A | 265 | 76.309 | −7.196 | 87.628 | 1.00 | 57.82 |
| 330 | CA | GLN | A | 265 | 76.197 | −7.443 | 89.058 | 1.00 | 53.76 |
| 331 | CB | GLN | A | 265 | 77.310 | −8.360 | 89.559 | 1.00 | 57.49 |
| 332 | CG | GLN | A | 265 | 76.829 | −9.357 | 90.602 | 1.00 | 67.60 |
| 333 | CD | GLN | A | 265 | 75.542 | −10.053 | 90.188 | 1.00 | 75.71 |
| 334 | OE1 | GLN | A | 265 | 74.446 | −9.525 | 90.398 | 1.00 | 82.27 |
| 335 | NE2 | GLN | A | 265 | 75.669 | −11.227 | 89.573 | 1.00 | 73.63 |
| 336 | C | GLN | A | 265 | 76.202 | −6.145 | 89.806 | 1.00 | 48.27 |
| 337 | O | GLN | A | 265 | 75.943 | −6.112 | 90.993 | 1.00 | 50.20 |
| 338 | N | GLN | A | 266 | 76.483 | −5.067 | 89.089 | 1.00 | 44.20 |
| 339 | CA | GLN | A | 266 | 76.457 | −3.726 | 89.667 | 1.00 | 39.57 |
| 340 | CB | GLN | A | 266 | 77.486 | −2.803 | 89.028 | 1.00 | 43.03 |
| 341 | CG | GLN | A | 266 | 78.772 | −2.635 | 89.794 | 1.00 | 53.90 |
| 342 | CD | GLN | A | 266 | 79.708 | −1.641 | 89.098 | 1.00 | 62.90 |
| 343 | OE1 | GLN | A | 266 | 79.394 | −0.451 | 89.007 | 1.00 | 60.13 |
| 344 | NE2 | GLN | A | 266 | 80.851 | −2.128 | 88.595 | 1.00 | 60.44 |
| 345 | C | GLN | A | 266 | 75.082 | −3.165 | 89.347 | 1.00 | 36.73 |
| 346 | O | GLN | A | 266 | 74.412 | −2.637 | 90.214 | 1.00 | 34.43 |
| 347 | N | ARG | A | 267 | 74.659 | −3.291 | 88.090 | 1.00 | 36.08 |
| 348 | CA | ARG | A | 267 | 73.365 | −2.768 | 87.683 | 1.00 | 34.02 |
| 349 | CB | ARG | A | 267 | 73.007 | −3.181 | 86.271 | 1.00 | 28.42 |
| 350 | CG | ARG | A | 267 | 73.888 | −2.569 | 85.248 | 1.00 | 32.75 |
| 351 | CD | ARG | A | 267 | 73.805 | −3.337 | 83.932 | 1.00 | 36.45 |
| 352 | NE | ARG | A | 267 | 74.755 | −2.825 | 82.947 | 1.00 | 46.52 |
| 353 | CZ | ARG | A | 267 | 75.042 | −3.431 | 81.810 | 1.00 | 45.30 |
| 354 | NH1 | ARG | A | 267 | 74.451 | −4.573 | 81.518 | 1.00 | 54.30 |
| 355 | NH2 | ARG | A | 267 | 75.914 | −2.882 | 80.978 | 1.00 | 54.13 |
| 356 | C | ARG | A | 267 | 72.354 | −3.321 | 88.616 | 1.00 | 32.85 |
| 357 | O | ARG | A | 267 | 71.542 | −2.585 | 89.167 | 1.00 | 38.40 |
| 358 | N | PHE | A | 268 | 72.413 | −4.626 | 88.826 | 1.00 | 29.74 |
| 359 | CA | PHE | A | 268 | 71.448 | −5.243 | 89.716 | 1.00 | 25.18 |
| 360 | CB | PHE | A | 268 | 71.669 | −6.741 | 89.728 | 1.00 | 26.06 |
| 361 | CG | PHE | A | 268 | 70.906 | −7.460 | 90.784 | 1.00 | 24.07 |
| 362 | CD1 | PHE | A | 268 | 71.363 | −7.455 | 92.103 | 1.00 | 23.22 |
| 363 | CD2 | PHE | A | 268 | 69.758 | −8.173 | 90.454 | 1.00 | 16.43 |
| 364 | CE1 | PHE | A | 268 | 70.688 | −8.150 | 93.084 | 1.00 | 31.48 |
| 365 | CE2 | PHE | A | 268 | 69.049 | −8.895 | 91.427 | 1.00 | 17.72 |
| 366 | CZ | PHE | A | 268 | 69.510 | −8.888 | 92.745 | 1.00 | 34.32 |
| 367 | C | PHE | A | 268 | 71.523 | −4.668 | 91.131 | 1.00 | 22.70 |
| 368 | O | PHE | A | 268 | 70.494 | −4.537 | 91.820 | 1.00 | 19.53 |
| 369 | N | ALA | A | 269 | 72.741 | −4.352 | 91.566 | 1.00 | 15.63 |
| 370 | CA | ALA | A | 269 | 72.930 | −3.772 | 92.891 | 1.00 | 16.25 |
| 371 | CB | ALA | A | 269 | 74.408 | −3.722 | 93.246 | 1.00 | 5.25 |
| 372 | C | ALA | A | 269 | 72.335 | −2.351 | 92.884 | 1.00 | 18.99 |
| 373 | O | ALA | A | 269 | 71.718 | −1.906 | 93.883 | 1.00 | 15.16 |
| 374 | N | HIS | A | 270 | 72.489 | −1.670 | 91.738 | 1.00 | 18.91 |
| 375 | CA | HIS | A | 270 | 71.965 | −0.321 | 91.562 | 1.00 | 19.16 |
| 376 | CB | HIS | A | 270 | 72.239 | 0.177 | 90.133 | 1.00 | 18.47 |
| 377 | CG | HIS | A | 270 | 71.953 | 1.641 | 89.932 | 1.00 | 24.67 |
| 378 | CD2 | HIS | A | 270 | 70.993 | 2.282 | 89.213 | 1.00 | 20.88 |
| 379 | ND1 | HIS | A | 270 | 72.663 | 2.630 | 90.579 | 1.00 | 21.21 |
| 380 | CE1 | HIS | A | 270 | 72.149 | 3.811 | 90.277 | 1.00 | 24.56 |
| 381 | NE2 | HIS | A | 270 | 71.134 | 3.626 | 89.449 | 1.00 | 23.56 |
| 382 | C | HIS | A | 270 | 70.444 | −0.265 | 91.869 | 1.00 | 20.09 |
| 383 | O | HIS | A | 270 | 69.997 | 0.578 | 92.671 | 1.00 | 19.20 |
| 384 | N | PHE | A | 271 | 69.670 | −1.161 | 91.246 | 1.00 | 17.60 |
| 385 | CA | PHE | A | 271 | 68.235 | −1.190 | 91.452 | 1.00 | 12.83 |
| 386 | CB | PHE | A | 271 | 67.558 | −2.153 | 90.474 | 1.00 | 13.28 |
| 387 | CG | PHE | A | 271 | 67.365 | −1.602 | 89.089 | 1.00 | 15.11 |
| 388 | CD1 | PHE | A | 271 | 66.499 | −2.201 | 88.224 | 1.00 | 21.67 |
| 389 | CD2 | PHE | A | 271 | 68.069 | −0.514 | 88.649 | 1.00 | 19.45 |
| 390 | CE1 | PHE | A | 271 | 66.340 | −1.715 | 86.923 | 1.00 | 33.27 |
| 391 | CE2 | PHE | A | 271 | 67.921 | −0.026 | 87.361 | 1.00 | 23.53 |
| 392 | CZ | PHE | A | 271 | 67.062 | −0.622 | 86.500 | 1.00 | 19.54 |
| 393 | C | PHE | A | 271 | 67.934 | −1.632 | 92.872 | 1.00 | 15.62 |
| 394 | O | PHE | A | 271 | 67.206 | −0.941 | 93.599 | 1.00 | 13.63 |
| 395 | N | THR | A | 272 | 68.508 | −2.771 | 93.272 | 1.00 | 17.50 |
| 396 | CA | THR | A | 272 | 68.275 | −3.325 | 94.605 | 1.00 | 21.01 |
| 397 | CB | THR | A | 272 | 69.110 | −4.569 | 94.864 | 1.00 | 21.69 |
| 398 | OG1 | THR | A | 272 | 70.479 | −4.289 | 94.562 | 1.00 | 21.21 |
| 399 | CG2 | THR | A | 272 | 68.589 | −5.712 | 94.031 | 1.00 | 22.74 |
| 400 | C | THR | A | 272 | 68.541 | −2.339 | 95.728 | 1.00 | 22.06 |
| 401 | O | THR | A | 272 | 67.720 | −2.256 | 96.694 | 1.00 | 13.99 |
| 402 | N | GLU | A | 273 | 69.659 | −1.598 | 95.593 | 1.00 | 21.82 |
| 403 | CA | GLU | A | 273 | 70.044 | −0.592 | 96.600 | 1.00 | 22.04 |
| 404 | CB | GLU | A | 273 | 71.505 | −0.134 | 96.432 | 1.00 | 16.72 |
| 405 | CG | GLU | A | 273 | 72.527 | −1.268 | 96.656 | 1.00 | 20.48 |
| 406 | CD | GLU | A | 273 | 73.954 | −0.888 | 96.301 | 1.00 | 32.58 |
| 407 | OE1 | GLU | A | 273 | 74.798 | −1.820 | 96.156 | 1.00 | 23.16 |
| 408 | OE2 | GLU | A | 273 | 74.237 | 0.335 | 96.163 | 1.00 | 34.71 |
| 409 | C | GLU | A | 273 | 69.114 | 0.604 | 96.571 | 1.00 | 18.71 |
| 410 | O | GLU | A | 273 | 68.849 | 1.207 | 97.616 | 1.00 | 20.97 |
| 411 | N | LEU | A | 274 | 68.595 | 0.926 | 95.385 | 1.00 | 16.51 |
| 412 | CA | LEU | A | 274 | 67.663 | 2.043 | 95.255 | 1.00 | 14.99 |
| 413 | CB | LEU | A | 274 | 67.495 | 2.463 | 93.796 | 1.00 | 11.12 |
| 414 | CG | LEU | A | 274 | 68.698 | 3.147 | 93.150 | 1.00 | 13.55 |
| 415 | CD1 | LEU | A | 274 | 68.600 | 3.124 | 91.642 | 1.00 | 5.25 |
| 416 | CD2 | LEU | A | 274 | 68.776 | 4.556 | 93.678 | 1.00 | 20.85 |
| 417 | C | LEU | A | 274 | 66.321 | 1.591 | 95.837 | 1.00 | 19.28 |
| 418 | O | LEU | A | 274 | 65.687 | 2.291 | 96.614 | 1.00 | 24.78 |
| 419 | N | ALA | A | 275 | 65.882 | 0.395 | 95.496 | 1.00 | 21.07 |
| 420 | CA | ALA | A | 275 | 64.624 | −0.091 | 96.054 | 1.00 | 20.24 |
| 421 | CB | ALA | A | 275 | 64.417 | −1.544 | 95.647 | 1.00 | 14.28 |
| 422 | C | ALA | A | 275 | 64.582 | 0.046 | 97.593 | 1.00 | 20.13 |
| 423 | O | ALA | A | 275 | 63.547 | 0.420 | 98.159 | 1.00 | 15.46 |
| 424 | N | ILE | A | 276 | 65.693 | −0.285 | 98.263 | 1.00 | 20.36 |
| 425 | CA | ILE | A | 276 | 65.769 | −0.177 | 99.725 | 1.00 | 20.48 |
| 426 | CB | ILE | A | 276 | 67.244 | −0.373 | 100.282 | 1.00 | 22.98 |
| 427 | CG2 | ILE | A | 276 | 67.357 | 0.091 | 101.688 | 1.00 | 5.25 |
| 428 | CG1 | ILE | A | 276 | 67.626 | −1.848 | 100.300 | 1.00 | 13.96 |
| 429 | CD1 | ILE | A | 276 | 69.078 | −2.061 | 100.525 | 1.00 | 5.37 |
| 430 | C | ILE | A | 276 | 65.271 | 1.199 | 100.119 | 1.00 | 22.12 |
| 431 | O | ILE | A | 276 | 64.304 | 1.330 | 100.848 | 1.00 | 26.12 |
| 432 | N | ILE | A | 277 | 65.932 | 2.233 | 99.630 | 1.00 | 21.56 |
| 433 | CA | ILE | A | 277 | 65.513 | 3.585 | 99.969 | 1.00 | 21.63 |
| 434 | CB | ILE | A | 277 | 66.224 | 4.638 | 99.059 | 1.00 | 21.92 |
| 435 | CG2 | ILE | A | 277 | 65.803 | 6.046 | 99.432 | 1.00 | 6.60 |
| 436 | CG1 | ILE | A | 277 | 67.733 | 4.455 | 99.182 | 1.00 | 19.32 |
| 437 | CD1 | ILE | A | 277 | 68.526 | 5.432 | 98.388 | 1.00 | 18.27 |
| 438 | C | ILE | A | 277 | 64.003 | 3.666 | 99.790 | 1.00 | 22.66 |
| 439 | O | ILE | A | 277 | 63.294 | 4.193 | 100.649 | 1.00 | 23.75 |
| 440 | N | SER | A | 278 | 63.512 | 3.128 | 98.675 | 1.00 | 21.69 |
| 441 | CA | SER | A | 278 | 62.074 | 3.158 | 98.421 | 1.00 | 25.15 |
| 442 | CB | SER | A | 278 | 61.729 | 2.477 | 97.092 | 1.00 | 27.78 |
| 443 | OG | SER | A | 278 | 60.337 | 2.601 | 96.802 | 1.00 | 14.94 |
| 444 | C | SER | A | 278 | 61.270 | 2.518 | 99.583 | 1.00 | 24.56 |
| 445 | O | SER | A | 278 | 60.235 | 3.081 | 100.039 | 1.00 | 26.24 |
| 446 | N | VAL | A | 279 | 61.737 | 1.371 | 100.074 | 1.00 | 19.27 |
| 447 | CA | VAL | A | 279 | 61.048 | 0.764 | 101.194 | 1.00 | 16.83 |
| 448 | CB | VAL | A | 279 | 61.719 | −0.529 | 101.651 | 1.00 | 9.75 |
| 449 | CG1 | VAL | A | 279 | 60.929 | −1.142 | 102.780 | 1.00 | 5.43 |
| 450 | CG2 | VAL | A | 279 | 61.760 | −1.491 | 100.509 | 1.00 | 7.30 |
| 451 | C | VAL | A | 279 | 61.004 | 1.741 | 102.366 | 1.00 | 17.92 |
| 452 | O | VAL | A | 279 | 59.959 | 1.894 | 103.019 | 1.00 | 20.05 |
| 453 | N | GLN | A | 280 | 62.118 | 2.432 | 102.616 | 1.00 | 17.87 |
| 454 | CA | GLN | A | 280 | 62.178 | 3.382 | 103.738 | 1.00 | 22.98 |
| 455 | CB | GLN | A | 280 | 63.613 | 3.887 | 103.939 | 1.00 | 22.68 |
| 456 | CG | GLN | A | 280 | 64.583 | 2.771 | 104.299 | 1.00 | 25.49 |
| 457 | CD | GLN | A | 280 | 66.035 | 3.158 | 104.103 | 1.00 | 29.97 |
| 458 | OE1 | GLN | A | 280 | 66.337 | 4.262 | 103.637 | 1.00 | 39.81 |
| 459 | NE2 | GLN | A | 280 | 66.951 | 2.246 | 104.459 | 1.00 | 28.32 |
| 460 | C | GLN | A | 280 | 61.232 | 4.566 | 103.550 | 1.00 | 21.90 |
| 461 | O | GLN | A | 280 | 60.701 | 5.078 | 104.521 | 1.00 | 13.34 |
| 462 | N | GLU | A | 281 | 61.018 | 4.977 | 102.292 | 1.00 | 23.40 |
| 463 | CA | GLU | A | 281 | 60.131 | 6.097 | 101.978 | 1.00 | 20.00 |
| 464 | CB | GLU | A | 281 | 60.261 | 6.487 | 100.530 | 1.00 | 14.61 |
| 465 | CG | GLU | A | 281 | 59.269 | 7.591 | 100.132 | 1.00 | 21.34 |
| 466 | CD | GLU | A | 281 | 59.364 | 7.998 | 98.655 | 1.00 | 17.98 |
| 467 | OE1 | GLU | A | 281 | 58.533 | 8.862 | 98.239 | 1.00 | 23.30 |
| 468 | OE2 | GLU | A | 281 | 60.253 | 7.464 | 97.918 | 1.00 | 23.51 |
| 469 | C | GLU | A | 281 | 58.705 | 5.622 | 102.209 | 1.00 | 22.73 |
| 470 | O | GLU | A | 281 | 57.868 | 6.333 | 102.846 | 1.00 | 19.39 |
| 471 | N | ILE | A | 282 | 58.425 | 4.420 | 101.679 | 1.00 | 20.62 |
| 472 | CA | ILE | A | 282 | 57.108 | 3.827 | 101.853 | 1.00 | 17.79 |
| 473 | CB | ILE | A | 282 | 57.063 | 2.501 | 101.196 | 1.00 | 12.76 |
| 474 | CG2 | ILE | A | 282 | 55.928 | 1.682 | 101.785 | 1.00 | 13.17 |
| 475 | CG1 | ILE | A | 282 | 56.895 | 2.717 | 99.685 | 1.00 | 10.52 |
| 476 | CD1 | ILE | A | 282 | 56.885 | 1.414 | 98.819 | 1.00 | 7.32 |
| 477 | C | ILE | A | 282 | 56.740 | 3.700 | 103.347 | 1.00 | 16.55 |
| 478 | O | ILE | A | 282 | 55.686 | 4.193 | 103.791 | 1.00 | 12.54 |
| 479 | N | VAL | A | 283 | 57.614 | 3.063 | 104.126 | 1.00 | 14.35 |
| 480 | CA | VAL | A | 283 | 57.353 | 2.949 | 105.545 | 1.00 | 17.95 |
| 481 | CB | VAL | A | 283 | 58.496 | 2.274 | 106.273 | 1.00 | 11.99 |
| 482 | CG1 | VAL | A | 283 | 58.266 | 2.361 | 107.794 | 1.00 | 5.25 |
| 483 | CG2 | VAL | A | 283 | 58.576 | 0.824 | 105.793 | 1.00 | 11.23 |
| 484 | C | VAL | A | 283 | 57.124 | 4.309 | 106.185 | 1.00 | 20.91 |
| 485 | O | VAL | A | 283 | 56.169 | 4.523 | 106.986 | 1.00 | 23.47 |
| 486 | N | ASP | A | 284 | 58.001 | 5.236 | 105.814 | 1.00 | 23.66 |
| 487 | CA | ASP | A | 284 | 57.948 | 6.601 | 106.326 | 1.00 | 22.91 |
| 488 | CB | ASP | A | 284 | 59.182 | 7.355 | 105.841 | 1.00 | 17.43 |
| 489 | CG | ASP | A | 284 | 59.127 | 8.811 | 106.144 | 1.00 | 22.55 |
| 490 | OD1 | ASP | A | 284 | 58.431 | 9.194 | 107.093 | 1.00 | 42.17 |
| 491 | OD2 | ASP | A | 284 | 59.783 | 9.607 | 105.443 | 1.00 | 36.89 |
| 492 | C | ASP | A | 284 | 56.623 | 7.306 | 105.955 | 1.00 | 23.76 |
| 493 | O | ASP | A | 284 | 56.040 | 8.009 | 106.788 | 1.00 | 27.90 |
| 494 | N | PHE | A | 285 | 56.123 | 7.086 | 104.744 | 1.00 | 17.48 |
| 495 | CA | PHE | A | 285 | 54.850 | 7.673 | 104.367 | 1.00 | 19.07 |
| 496 | CB | PHE | A | 285 | 54.732 | 7.647 | 102.853 | 1.00 | 16.30 |
| 497 | CG | PHE | A | 285 | 53.383 | 8.069 | 102.346 | 1.00 | 14.98 |
| 498 | CD1 | PHE | A | 285 | 52.991 | 9.398 | 102.422 | 1.00 | 13.11 |
| 499 | CD2 | PHE | A | 285 | 52.499 | 7.135 | 101.819 | 1.00 | 14.66 |
| 500 | CE1 | PHE | A | 285 | 51.711 | 9.815 | 101.980 | 1.00 | 14.89 |
| 501 | CE2 | PHE | A | 285 | 51.214 | 7.526 | 101.367 | 1.00 | 23.01 |
| 502 | CZ | PHE | A | 285 | 50.814 | 8.886 | 101.448 | 1.00 | 5.25 |
| 503 | C | PHE | A | 285 | 53.574 | 6.996 | 104.984 | 1.00 | 23.61 |
| 504 | O | PHE | A | 285 | 52.560 | 7.681 | 105.230 | 1.00 | 22.21 |
| 505 | N | ALA | A | 286 | 53.596 | 5.665 | 105.162 | 1.00 | 23.51 |
| 506 | CA | ALA | A | 286 | 52.444 | 4.964 | 105.757 | 1.00 | 27.50 |
| 507 | CB | ALA | A | 286 | 52.641 | 3.449 | 105.744 | 1.00 | 25.14 |
| 508 | C | ALA | A | 286 | 52.272 | 5.429 | 107.205 | 1.00 | 26.35 |
| 509 | O | ALA | A | 286 | 51.171 | 5.387 | 107.743 | 1.00 | 24.23 |
| 510 | N | LYS | A | 287 | 53.376 | 5.856 | 107.812 | 1.00 | 21.64 |
| 511 | CA | LYS | A | 287 | 53.385 | 6.337 | 109.173 | 1.00 | 20.78 |
| 512 | CB | LYS | A | 287 | 54.829 | 6.569 | 109.627 | 1.00 | 32.68 |
| 513 | CG | LYS | A | 287 | 55.557 | 5.394 | 110.287 | 1.00 | 16.23 |
| 514 | CD | LYS | A | 287 | 56.916 | 5.890 | 110.820 | 1.00 | 6.38 |
| 515 | CE | LYS | A | 287 | 57.643 | 4.797 | 111.544 | 1.00 | 20.16 |
| 516 | NZ | LYS | A | 287 | 58.945 | 5.307 | 112.046 | 1.00 | 34.26 |
| 517 | C | LYS | A | 287 | 52.650 | 7.648 | 109.252 | 1.00 | 18.44 |
| 518 | O | LYS | A | 287 | 52.266 | 8.100 | 110.313 | 1.00 | 11.43 |
| 519 | N | GLN | A | 288 | 52.481 | 8.263 | 108.100 | 1.00 | 21.42 |
| 520 | CA | GLN | A | 288 | 51.810 | 9.553 | 108.006 | 1.00 | 22.66 |
| 521 | CB | GLN | A | 288 | 52.539 | 10.411 | 106.980 | 1.00 | 29.15 |
| 522 | CG | GLN | A | 288 | 54.018 | 10.573 | 107.234 | 1.00 | 42.69 |
| 523 | CD | GLN | A | 288 | 54.304 | 11.694 | 108.176 | 1.00 | 49.48 |
| 524 | OE1 | GLN | A | 288 | 55.423 | 12.173 | 108.258 | 1.00 | 74.17 |
| 525 | NE2 | GLN | A | 288 | 53.292 | 12.138 | 108.883 | 1.00 | 51.28 |
| 526 | C | GLN | A | 288 | 50.350 | 9.409 | 107.581 | 1.00 | 21.76 |
| 527 | O | GLN | A | 288 | 49.603 | 10.383 | 107.656 | 1.00 | 27.23 |
| 528 | N | VAL | A | 289 | 49.953 | 8.211 | 107.129 | 1.00 | 19.79 |
| 529 | CA | VAL | A | 289 | 48.580 | 7.973 | 106.681 | 1.00 | 15.70 |
| 530 | CB | VAL | A | 289 | 48.488 | 6.742 | 105.759 | 1.00 | 13.08 |
| 531 | CG1 | VAL | A | 289 | 47.074 | 6.556 | 105.298 | 1.00 | 6.01 |
| 532 | CG2 | VAL | A | 289 | 49.404 | 6.935 | 104.572 | 1.00 | 15.89 |
| 533 | C | VAL | A | 289 | 47.722 | 7.798 | 107.921 | 1.00 | 14.36 |
| 534 | O | VAL | A | 289 | 47.875 | 6.848 | 108.678 | 1.00 | 10.54 |
| 535 | N | PRO | A | 290 | 46.835 | 8.766 | 108.171 | 1.00 | 14.08 |
| 536 | CD | PRO | A | 290 | 46.585 | 9.963 | 107.354 | 1.00 | 17.19 |
| 537 | CA | PRO | A | 290 | 45.948 | 8.749 | 109.327 | 1.00 | 15.24 |
| 538 | CB | PRO | A | 290 | 44.921 | 9.810 | 108.977 | 1.00 | 5.72 |
| 539 | CG | PRO | A | 290 | 45.719 | 10.783 | 108.275 | 1.00 | 16.05 |
| 540 | C | PRO | A | 290 | 45.321 | 7.387 | 109.524 | 1.00 | 21.69 |
| 541 | O | PRO | A | 290 | 44.658 | 6.880 | 108.633 | 1.00 | 27.78 |
| 542 | N | GLY | A | 291 | 45.508 | 6.827 | 110.715 | 1.00 | 21.98 |
| 543 | CA | GLY | A | 291 | 44.942 | 5.550 | 111.030 | 1.00 | 16.77 |
| 544 | C | GLY | A | 291 | 45.968 | 4.456 | 111.051 | 1.00 | 23.95 |
| 545 | O | GLY | A | 291 | 45.844 | 3.533 | 111.864 | 1.00 | 27.61 |
| 546 | N | PHE | A | 292 | 46.986 | 4.554 | 110.191 | 1.00 | 23.02 |
| 547 | CA | PHE | A | 292 | 47.999 | 3.503 | 110.135 | 1.00 | 23.53 |
| 548 | CB | PHE | A | 292 | 49.080 | 3.824 | 109.096 | 1.00 | 9.73 |
| 549 | CG | PHE | A | 292 | 49.971 | 2.675 | 108.803 | 1.00 | 5.25 |
| 550 | CD1 | PHE | A | 292 | 49.524 | 1.613 | 108.044 | 1.00 | 5.25 |
| 551 | CD2 | PHE | A | 292 | 51.234 | 2.610 | 109.359 | 1.00 | 9.44 |
| 552 | CE1 | PHE | A | 292 | 50.337 | 0.490 | 107.860 | 1.00 | 8.02 |
| 553 | CE2 | PHE | A | 292 | 52.089 | 1.473 | 109.177 | 1.00 | 5.29 |
| 554 | CZ | PHE | A | 292 | 51.647 | 0.429 | 108.441 | 1.00 | 6.77 |
| 555 | C | PHE | A | 292 | 48.654 | 3.216 | 111.486 | 1.00 | 25.20 |
| 556 | O | PHE | A | 292 | 48.712 | 2.072 | 111.939 | 1.00 | 28.15 |
| 557 | N | LEU | A | 293 | 49.125 | 4.250 | 112.163 | 1.00 | 26.45 |
| 558 | CA | LEU | A | 293 | 49.793 | 4.021 | 113.451 | 1.00 | 25.06 |
| 559 | CB | LEU | A | 293 | 50.565 | 5.278 | 113.875 | 1.00 | 12.77 |
| 560 | CG | LEU | A | 293 | 51.911 | 5.434 | 113.153 | 1.00 | 13.71 |
| 561 | CD1 | LEU | A | 293 | 52.619 | 6.647 | 113.686 | 1.00 | 20.57 |
| 562 | CD2 | LEU | A | 293 | 52.784 | 4.213 | 113.389 | 1.00 | 17.42 |
| 563 | C | LEU | A | 293 | 48.902 | 3.552 | 114.590 | 1.00 | 26.84 |
| 564 | O | LEU | A | 293 | 49.380 | 3.227 | 115.662 | 1.00 | 27.75 |
| 565 | N | GLN | A | 294 | 47.603 | 3.526 | 114.366 | 1.00 | 28.94 |
| 566 | CA | GLN | A | 294 | 46.703 | 3.072 | 115.406 | 1.00 | 29.14 |
| 567 | CB | GLN | A | 294 | 45.335 | 3.721 | 115.199 | 1.00 | 32.40 |
| 568 | CG | GLN | A | 294 | 45.373 | 5.200 | 115.308 | 1.00 | 34.00 |
| 569 | CD | GLN | A | 294 | 44.058 | 5.848 | 114.902 | 1.00 | 47.60 |
| 570 | OE1 | GLN | A | 294 | 43.968 | 7.088 | 114.834 | 1.00 | 47.41 |
| 571 | NE2 | GLN | A | 294 | 43.028 | 5.020 | 114.620 | 1.00 | 31.54 |
| 572 | C | GLN | A | 294 | 46.585 | 1.514 | 115.429 | 1.00 | 29.82 |
| 573 | O | GLN | A | 294 | 46.088 | 0.931 | 116.413 | 1.00 | 27.35 |
| 574 | N | LEU | A | 295 | 47.031 | 0.852 | 114.350 | 1.00 | 24.78 |
| 575 | CA | LEU | A | 295 | 46.983 | −0.605 | 114.275 | 1.00 | 22.06 |
| 576 | CB | LEU | A | 295 | 47.075 | −1.093 | 112.820 | 1.00 | 15.09 |
| 577 | CG | LEU | A | 295 | 45.917 | −0.816 | 111.856 | 1.00 | 8.95 |
| 578 | CD1 | LEU | A | 295 | 46.207 | −1.416 | 110.509 | 1.00 | 5.25 |
| 579 | CD2 | LEU | A | 295 | 44.645 | −1.407 | 112.431 | 1.00 | 7.77 |
| 580 | C | LEU | A | 295 | 48.151 | −1.169 | 115.083 | 1.00 | 24.15 |
| 581 | O | LEU | A | 295 | 48.938 | −0.432 | 115.679 | 1.00 | 25.53 |
| 582 | N | GLY | A | 296 | 48.250 | −2.484 | 115.123 | 1.00 | 25.20 |
| 583 | CA | GLY | A | 296 | 49.330 | −3.105 | 115.860 | 1.00 | 25.00 |
| 584 | C | GLY | A | 296 | 50.530 | −3.319 | 114.947 | 1.00 | 28.85 |
| 585 | O | GLY | A | 296 | 50.389 | −3.560 | 113.738 | 1.00 | 30.52 |
| 586 | N | ARG | A | 297 | 51.723 | −3.235 | 115.527 | 1.00 | 27.91 |
| 587 | CA | ARG | A | 297 | 52.934 | −3.416 | 114.771 | 1.00 | 26.54 |
| 588 | CB | ARG | A | 297 | 54.135 | −3.450 | 115.724 | 1.00 | 26.53 |
| 589 | CG | ARG | A | 297 | 54.646 | −2.058 | 116.142 | 1.00 | 34.70 |
| 590 | CD | ARG | A | 297 | 55.192 | −1.263 | 114.973 | 1.00 | 43.40 |
| 591 | NE | ARG | A | 297 | 55.933 | −0.085 | 115.407 | 1.00 | 49.04 |
| 592 | CZ | ARG | A | 297 | 55.375 | 1.010 | 115.898 | 1.00 | 44.17 |
| 593 | NH1 | ARG | A | 297 | 54.064 | 1.073 | 116.017 | 1.00 | 58.77 |
| 594 | NH2 | ARG | A | 297 | 56.132 | 2.042 | 116.257 | 1.00 | 42.14 |
| 595 | C | ARG | A | 297 | 52.874 | −4.661 | 113.882 | 1.00 | 25.01 |
| 596 | O | ARG | A | 297 | 53.394 | −4.663 | 112.766 | 1.00 | 26.94 |
| 597 | N | GLU | A | 298 | 52.218 | −5.708 | 114.355 | 1.00 | 22.09 |
| 598 | CA | GLU | A | 298 | 52.115 | −6.923 | 113.556 | 1.00 | 24.63 |
| 599 | CB | GLU | A | 298 | 51.327 | −8.010 | 114.290 | 1.00 | 28.37 |
| 600 | CG | GLU | A | 298 | 51.848 | −8.340 | 115.658 | 1.00 | 37.88 |
| 601 | CD | GLU | A | 298 | 51.292 | −7.409 | 116.740 | 1.00 | 55.37 |
| 602 | OE1 | GLU | A | 298 | 50.070 | −7.452 | 117.028 | 1.00 | 59.05 |
| 603 | OE2 | GLU | A | 298 | 52.076 | −6.614 | 117.307 | 1.00 | 70.02 |
| 604 | C | GLU | A | 298 | 51.389 | −6.558 | 112.273 | 1.00 | 23.66 |
| 605 | O | GLU | A | 298 | 51.927 | −6.676 | 111.158 | 1.00 | 20.63 |
| 606 | N | ASP | A | 299 | 50.155 | −6.101 | 112.429 | 1.00 | 19.84 |
| 607 | CA | ASP | A | 299 | 49.390 | −5.712 | 111.253 | 1.00 | 21.20 |
| 608 | CB | ASP | A | 299 | 47.983 | −5.270 | 111.667 | 1.00 | 16.47 |
| 609 | CG | ASP | A | 299 | 47.028 | −6.459 | 111.883 | 1.00 | 22.01 |
| 610 | OD1 | ASP | A | 299 | 47.464 | −7.631 | 111.711 | 1.00 | 20.27 |
| 611 | OD2 | ASP | A | 299 | 45.834 | −6.227 | 112.223 | 1.00 | 17.13 |
| 612 | C | ASP | A | 299 | 50.088 | −4.619 | 110.406 | 1.00 | 18.39 |
| 613 | O | ASP | A | 299 | 49.886 | −4.542 | 109.198 | 1.00 | 18.30 |
| 614 | N | GLN | A | 300 | 50.916 | −3.784 | 111.024 | 1.00 | 17.35 |
| 615 | CA | GLN | A | 300 | 51.595 | −2.747 | 110.259 | 1.00 | 15.73 |
| 616 | CB | GLN | A | 300 | 52.376 | −1.789 | 111.169 | 1.00 | 9.88 |
| 617 | CG | GLN | A | 300 | 51.477 | −0.866 | 111.981 | 1.00 | 27.45 |
| 618 | CD | GLN | A | 300 | 52.229 | 0.097 | 112.877 | 1.00 | 13.50 |
| 619 | OE1 | GLN | A | 300 | 51.702 | 0.566 | 113.878 | 1.00 | 23.51 |
| 620 | NE2 | GLN | A | 300 | 53.458 | 0.379 | 112.530 | 1.00 | 24.69 |
| 621 | C | GLN | A | 300 | 52.565 | −3.470 | 109.338 | 1.00 | 17.34 |
| 622 | O | GLN | A | 300 | 52.778 | −3.092 | 108.182 | 1.00 | 18.14 |
| 623 | N | ILE | A | 301 | 53.162 | −4.533 | 109.843 | 1.00 | 12.85 |
| 624 | CA | ILE | A | 301 | 54.101 | −5.236 | 109.016 | 1.00 | 14.59 |
| 625 | CB | ILE | A | 301 | 54.976 | −6.165 | 109.893 | 1.00 | 15.16 |
| 626 | CG2 | ILE | A | 301 | 55.820 | −7.091 | 109.001 | 1.00 | 5.25 |
| 627 | CG1 | ILE | A | 301 | 55.793 | −5.274 | 110.855 | 1.00 | 8.71 |
| 628 | CD1 | ILE | A | 301 | 56.769 | −5.993 | 111.737 | 1.00 | 10.92 |
| 629 | C | ILE | A | 301 | 53.384 | −5.993 | 107.909 | 1.00 | 15.65 |
| 630 | O | ILE | A | 301 | 53.741 | −5.875 | 106.766 | 1.00 | 12.48 |
| 631 | N | ALA | A | 302 | 52.353 | −6.747 | 108.274 | 1.00 | 20.32 |
| 632 | CA | ALA | A | 302 | 51.576 | −7.519 | 107.322 | 1.00 | 19.51 |
| 633 | CB | ALA | A | 302 | 50.314 | −8.081 | 107.978 | 1.00 | 20.40 |
| 634 | C | ALA | A | 302 | 51.188 | −6.645 | 106.161 | 1.00 | 19.30 |
| 635 | O | ALA | A | 302 | 51.409 | −7.008 | 104.998 | 1.00 | 21.12 |
| 636 | N | LEU | A | 303 | 50.603 | −5.497 | 106.477 | 1.00 | 16.93 |
| 637 | CA | LEU | A | 303 | 50.151 | −4.565 | 105.447 | 1.00 | 21.31 |
| 638 | CB | LEU | A | 303 | 49.373 | −3.409 | 106.101 | 1.00 | 13.13 |
| 639 | CG | LEU | A | 303 | 48.157 | −3.919 | 106.854 | 1.00 | 12.92 |
| 640 | CD1 | LEU | A | 303 | 47.499 | −2.757 | 107.493 | 1.00 | 18.24 |
| 641 | CD2 | LEU | A | 303 | 47.195 | −4.658 | 105.930 | 1.00 | 5.25 |
| 642 | C | LEU | A | 303 | 51.288 | −3.996 | 104.578 | 1.00 | 21.60 |
| 643 | O | LEU | A | 303 | 51.241 | −4.018 | 103.347 | 1.00 | 23.50 |
| 644 | N | LEU | A | 304 | 52.313 | −3.496 | 105.240 | 1.00 | 20.10 |
| 645 | CA | LEU | A | 304 | 53.445 | −2.897 | 104.567 | 1.00 | 17.11 |
| 646 | CB | LEU | A | 304 | 54.343 | −2.249 | 105.620 | 1.00 | 11.01 |
| 647 | CG | LEU | A | 304 | 54.510 | −0.741 | 105.558 | 1.00 | 18.49 |
| 648 | CD1 | LEU | A | 304 | 55.287 | −0.461 | 104.307 | 1.00 | 44.23 |
| 649 | CD2 | LEU | A | 304 | 53.181 | −0.015 | 105.462 | 1.00 | 20.96 |
| 650 | C | LEU | A | 304 | 54.208 | −3.921 | 103.748 | 1.00 | 16.89 |
| 651 | O | LEU | A | 304 | 54.649 | −3.658 | 102.635 | 1.00 | 20.67 |
| 652 | N | LYS | A | 305 | 54.360 | −5.109 | 104.288 | 1.00 | 13.73 |
| 653 | CA | LYS | A | 305 | 55.091 | −6.157 | 103.578 | 1.00 | 19.77 |
| 654 | CB | LYS | A | 305 | 55.291 | −7.356 | 104.527 | 1.00 | 20.81 |
| 655 | CG | LYS | A | 305 | 55.946 | −8.565 | 103.905 | 1.00 | 13.19 |
| 656 | CD | LYS | A | 305 | 55.916 | −9.726 | 104.870 | 1.00 | 20.63 |
| 657 | CE | LYS | A | 305 | 56.306 | −11.057 | 104.228 | 1.00 | 12.06 |
| 658 | NZ | LYS | A | 305 | 57.703 | −11.055 | 103.799 | 1.00 | 19.00 |
| 659 | C | LYS | A | 305 | 54.438 | −6.634 | 102.250 | 1.00 | 21.51 |
| 660 | O | LYS | A | 305 | 55.068 | −7.200 | 101.387 | 1.00 | 18.05 |
| 661 | N | ALA | A | 306 | 53.155 | −6.410 | 102.087 | 1.00 | 25.21 |
| 662 | CA | ALA | A | 306 | 52.491 | −6.827 | 100.864 | 1.00 | 23.27 |
| 663 | CB | ALA | A | 306 | 51.124 | −7.438 | 101.174 | 1.00 | 17.32 |
| 664 | C | ALA | A | 306 | 52.298 | −5.650 | 99.914 | 1.00 | 23.06 |
| 665 | O | ALA | A | 306 | 52.172 | −5.875 | 98.710 | 1.00 | 24.75 |
| 666 | N | SER | A | 307 | 52.287 | −4.405 | 100.412 | 1.00 | 16.72 |
| 667 | CA | SER | A | 307 | 52.036 | −3.328 | 99.481 | 1.00 | 18.89 |
| 668 | CB | SER | A | 307 | 51.075 | −2.280 | 100.087 | 1.00 | 18.39 |
| 669 | OG | SER | A | 307 | 51.577 | −1.640 | 101.231 | 1.00 | 27.27 |
| 670 | C | SER | A | 307 | 53.252 | −2.673 | 98.893 | 1.00 | 18.47 |
| 671 | O | SER | A | 307 | 53.137 | −1.896 | 97.952 | 1.00 | 17.24 |
| 672 | N | THR | A | 308 | 54.418 | −3.007 | 99.431 | 1.00 | 20.42 |
| 673 | CA | THR | A | 308 | 55.652 | −2.412 | 98.961 | 1.00 | 20.33 |
| 674 | CB | THR | A | 308 | 56.857 | −2.970 | 99.677 | 1.00 | 26.07 |
| 675 | OG1 | THR | A | 308 | 56.723 | −2.675 | 101.072 | 1.00 | 15.84 |
| 676 | CG2 | THR | A | 308 | 58.165 | −2.380 | 99.109 | 1.00 | 11.95 |
| 677 | C | THR | A | 308 | 55.806 | −2.623 | 97.491 | 1.00 | 24.21 |
| 678 | O | THR | A | 308 | 55.949 | −1.639 | 96.768 | 1.00 | 31.09 |
| 679 | N | ILE | A | 309 | 55.736 | −3.865 | 97.008 | 1.00 | 22.05 |
| 680 | CA | ILE | A | 309 | 55.885 | −4.058 | 95.541 | 1.00 | 21.97 |
| 681 | CB | ILE | A | 309 | 55.970 | −5.540 | 95.155 | 1.00 | 10.57 |
| 682 | CG2 | ILE | A | 309 | 54.599 | −6.187 | 95.213 | 1.00 | 10.35 |
| 683 | CG1 | ILE | A | 309 | 56.457 | −5.655 | 93.746 | 1.00 | 11.00 |
| 684 | CD1 | ILE | A | 309 | 57.624 | −4.806 | 93.430 | 1.00 | 15.31 |
| 685 | C | ILE | A | 309 | 54.741 | −3.384 | 94.723 | 1.00 | 24.31 |
| 686 | O | ILE | A | 309 | 54.955 | −2.796 | 93.647 | 1.00 | 26.07 |
| 687 | N | GLU | A | 310 | 53.531 | −3.417 | 95.257 | 1.00 | 24.15 |
| 688 | CA | GLU | A | 310 | 52.445 | −2.787 | 94.548 | 1.00 | 25.27 |
| 689 | CB | GLU | A | 310 | 51.100 | −3.217 | 95.156 | 1.00 | 20.97 |
| 690 | CG | GLU | A | 310 | 50.768 | −4.656 | 94.840 | 1.00 | 23.89 |
| 691 | CD | GLU | A | 310 | 49.495 | −5.121 | 95.467 | 1.00 | 28.73 |
| 692 | OE1 | GLU | A | 310 | 48.483 | −4.411 | 95.287 | 1.00 | 30.31 |
| 693 | OE2 | GLU | A | 310 | 49.510 | −6.199 | 96.125 | 1.00 | 29.66 |
| 694 | C | GLU | A | 310 | 52.610 | −1.262 | 94.528 | 1.00 | 23.80 |
| 695 | O | GLU | A | 310 | 52.459 | −0.630 | 93.456 | 1.00 | 22.71 |
| 696 | N | ILE | A | 311 | 52.925 | −0.688 | 95.698 | 1.00 | 21.18 |
| 697 | CA | ILE | A | 311 | 53.100 | 0.754 | 95.834 | 1.00 | 17.78 |
| 698 | CB | ILE | A | 311 | 53.420 | 1.153 | 97.270 | 1.00 | 22.01 |
| 699 | CG2 | ILE | A | 311 | 53.731 | 2.670 | 97.360 | 1.00 | 14.47 |
| 700 | CG1 | ILE | A | 311 | 52.256 | 0.772 | 98.180 | 1.00 | 20.79 |
| 701 | CD1 | ILE | A | 311 | 52.456 | 1.225 | 99.620 | 1.00 | 5.25 |
| 702 | C | ILE | A | 311 | 54.264 | 1.183 | 94.951 | 1.00 | 17.83 |
| 703 | O | ILE | A | 311 | 54.210 | 2.226 | 94.286 | 1.00 | 17.78 |
| 704 | N | MET | A | 312 | 55.310 | 0.367 | 94.914 | 1.00 | 12.27 |
| 705 | CA | MET | A | 312 | 56.447 | 0.689 | 94.053 | 1.00 | 11.78 |
| 706 | CB | MET | A | 312 | 57.513 | −0.410 | 94.128 | 1.00 | 8.84 |
| 707 | CG | MET | A | 312 | 58.406 | −0.330 | 95.372 | 1.00 | 8.36 |
| 708 | SD | MET | A | 312 | 59.459 | −1.748 | 95.526 | 1.00 | 13.68 |
| 709 | CE | MET | A | 312 | 60.895 | −1.101 | 96.293 | 1.00 | 30.13 |
| 710 | C | MET | A | 312 | 55.954 | 0.817 | 92.605 | 1.00 | 15.28 |
| 711 | O | MET | A | 312 | 56.238 | 1.815 | 91.939 | 1.00 | 14.23 |
| 712 | N | LEU | A | 313 | 55.214 | −0.196 | 92.126 | 1.00 | 17.47 |
| 713 | CA | LEU | A | 313 | 54.678 | −0.171 | 90.750 | 1.00 | 12.93 |
| 714 | CB | LEU | A | 313 | 53.984 | −1.497 | 90.424 | 1.00 | 6.84 |
| 715 | CG | LEU | A | 313 | 54.946 | −2.670 | 90.488 | 1.00 | 12.50 |
| 716 | CD1 | LEU | A | 313 | 54.187 | −3.949 | 90.340 | 1.00 | 16.17 |
| 717 | CD2 | LEU | A | 313 | 55.954 | −2.535 | 89.413 | 1.00 | 8.29 |
| 718 | C | LEU | A | 313 | 53.699 | 1.012 | 90.568 | 1.00 | 14.27 |
| 719 | O | LEU | A | 313 | 53.483 | 1.516 | 89.448 | 1.00 | 9.22 |
| 720 | N | LEU | A | 314 | 53.078 | 1.461 | 91.656 | 1.00 | 11.10 |
| 721 | CA | LEU | A | 314 | 52.211 | 2.593 | 91.465 | 1.00 | 12.84 |
| 722 | CB | LEU | A | 314 | 51.379 | 2.877 | 92.725 | 1.00 | 6.93 |
| 723 | CG | LEU | A | 314 | 50.068 | 2.130 | 92.863 | 1.00 | 6.99 |
| 724 | CD1 | LEU | A | 314 | 49.457 | 2.319 | 94.173 | 1.00 | 5.25 |
| 725 | CD2 | LEU | A | 314 | 49.126 | 2.632 | 91.850 | 1.00 | 7.30 |
| 726 | C | LEU | A | 314 | 53.155 | 3.777 | 91.119 | 1.00 | 12.77 |
| 727 | O | LEU | A | 314 | 52.982 | 4.460 | 90.105 | 1.00 | 11.50 |
| 728 | N | GLU | A | 315 | 54.177 | 3.961 | 91.950 | 1.00 | 11.87 |
| 729 | CA | GLU | A | 315 | 55.124 | 5.042 | 91.770 | 1.00 | 16.53 |
| 730 | CB | GLU | A | 315 | 56.120 | 5.039 | 92.932 | 1.00 | 11.89 |
| 731 | CG | GLU | A | 315 | 55.605 | 5.607 | 94.194 | 1.00 | 5.25 |
| 732 | CD | GLU | A | 315 | 55.595 | 7.090 | 94.201 | 1.00 | 10.85 |
| 733 | OE1 | GLU | A | 315 | 55.182 | 7.702 | 93.176 | 1.00 | 8.77 |
| 734 | OE2 | GLU | A | 315 | 55.999 | 7.645 | 95.248 | 1.00 | 17.41 |
| 735 | C | GLU | A | 315 | 55.865 | 4.976 | 90.426 | 1.00 | 16.97 |
| 736 | O | GLU | A | 315 | 56.052 | 6.002 | 89.730 | 1.00 | 17.98 |
| 737 | N | THR | A | 316 | 56.293 | 3.768 | 90.083 | 1.00 | 17.59 |
| 738 | CA | THR | A | 316 | 56.990 | 3.522 | 88.833 | 1.00 | 17.43 |
| 739 | CB | THR | A | 316 | 57.280 | 2.030 | 88.672 | 1.00 | 17.51 |
| 740 | OG1 | THR | A | 316 | 57.863 | 1.527 | 89.884 | 1.00 | 30.83 |
| 741 | CG2 | THR | A | 316 | 58.265 | 1.826 | 87.594 | 1.00 | 18.88 |
| 742 | C | THR | A | 316 | 56.081 | 4.003 | 87.710 | 1.00 | 11.80 |
| 743 | O | THR | A | 316 | 56.517 | 4.648 | 86.758 | 1.00 | 9.81 |
| 744 | N | ALA | A | 317 | 54.793 | 3.735 | 87.862 | 1.00 | 10.77 |
| 745 | CA | ALA | A | 317 | 53.840 | 4.147 | 86.849 | 1.00 | 16.37 |
| 746 | CB | ALA | A | 317 | 52.475 | 3.546 | 87.136 | 1.00 | 5.38 |
| 747 | C | ALA | A | 317 | 53.744 | 5.667 | 86.802 | 1.00 | 18.32 |
| 748 | O | ALA | A | 317 | 53.894 | 6.257 | 85.754 | 1.00 | 19.63 |
| 749 | N | ARG | A | 318 | 53.471 | 6.288 | 87.944 | 1.00 | 19.01 |
| 750 | CA | ARG | A | 318 | 53.340 | 7.728 | 88.018 | 1.00 | 20.43 |
| 751 | CB | ARG | A | 318 | 53.127 | 8.163 | 89.480 | 1.00 | 17.68 |
| 752 | CG | ARG | A | 318 | 53.723 | 9.553 | 89.752 | 1.00 | 26.39 |
| 753 | CD | ARG | A | 318 | 53.215 | 10.206 | 91.009 | 1.00 | 28.62 |
| 754 | NE | ARG | A | 318 | 53.609 | 9.530 | 92.240 | 1.00 | 10.50 |
| 755 | CZ | ARG | A | 318 | 53.102 | 9.861 | 93.417 | 1.00 | 16.96 |
| 756 | NH1 | ARG | A | 318 | 52.228 | 10.843 | 93.456 | 1.00 | 13.30 |
| 757 | NH2 | ARG | A | 318 | 53.424 | 9.199 | 94.539 | 1.00 | 11.73 |
| 758 | C | ARG | A | 318 | 54.565 | 8.450 | 87.438 | 1.00 | 22.34 |
| 759 | O | ARG | A | 318 | 54.467 | 9.564 | 86.933 | 1.00 | 21.25 |
| 760 | N | ARG | A | 319 | 55.713 | 7.793 | 87.488 | 1.00 | 21.35 |
| 761 | CA | ARG | A | 319 | 56.956 | 8.375 | 87.032 | 1.00 | 17.87 |
| 762 | CB | ARG | A | 319 | 58.045 | 7.931 | 88.022 | 1.00 | 20.75 |
| 763 | CG | ARG | A | 319 | 58.387 | 8.951 | 89.050 | 1.00 | 24.12 |
| 764 | CD | ARG | A | 319 | 58.545 | 8.402 | 90.431 | 1.00 | 17.36 |
| 765 | NE | ARG | A | 319 | 58.607 | 9.508 | 91.410 | 1.00 | 32.94 |
| 766 | CZ | ARG | A | 319 | 57.991 | 9.447 | 92.577 | 1.00 | 33.79 |
| 767 | NH1 | ARG | A | 319 | 57.317 | 8.346 | 92.849 | 1.00 | 43.26 |
| 768 | NH2 | ARG | A | 319 | 58.024 | 10.456 | 93.438 | 1.00 | 52.22 |
| 769 | C | ARG | A | 319 | 57.323 | 7.982 | 85.620 | 1.00 | 18.49 |
| 770 | O | ARG | A | 319 | 58.478 | 8.158 | 85.184 | 1.00 | 20.42 |
| 771 | N | TYR | A | 320 | 56.350 | 7.422 | 84.906 | 1.00 | 18.20 |
| 772 | CA | TYR | A | 320 | 56.550 | 6.958 | 83.520 | 1.00 | 14.45 |
| 773 | CB | TYR | A | 320 | 55.609 | 5.815 | 83.236 | 1.00 | 13.95 |
| 774 | CG | TYR | A | 320 | 55.706 | 5.312 | 81.847 | 1.00 | 8.19 |
| 775 | CD1 | TYR | A | 320 | 54.719 | 5.611 | 80.913 | 1.00 | 8.08 |
| 776 | CE1 | TYR | A | 320 | 54.753 | 5.056 | 79.620 | 1.00 | 17.06 |
| 777 | CD2 | TYR | A | 320 | 56.756 | 4.459 | 81.475 | 1.00 | 19.62 |
| 778 | CE2 | TYR | A | 320 | 56.819 | 3.888 | 80.178 | 1.00 | 18.44 |
| 779 | CZ | TYR | A | 320 | 55.803 | 4.187 | 79.265 | 1.00 | 17.55 |
| 780 | OH | TYR | A | 320 | 55.806 | 3.578 | 78.037 | 1.00 | 21.89 |
| 781 | C | TYR | A | 320 | 56.333 | 8.003 | 82.467 | 1.00 | 13.03 |
| 782 | O | TYR | A | 320 | 55.356 | 8.730 | 82.499 | 1.00 | 9.85 |
| 783 | N | ASN | A | 321 | 57.247 | 8.044 | 81.518 | 1.00 | 17.48 |
| 784 | CA | ASN | A | 321 | 57.208 | 9.010 | 80.425 | 1.00 | 24.17 |
| 785 | CB | ASN | A | 321 | 58.621 | 9.556 | 80.231 | 1.00 | 22.33 |
| 786 | CG | ASN | A | 321 | 58.745 | 10.434 | 79.018 | 1.00 | 35.24 |
| 787 | OD1 | ASN | A | 321 | 58.104 | 10.210 | 77.977 | 1.00 | 42.91 |
| 788 | ND2 | ASN | A | 321 | 59.602 | 11.432 | 79.126 | 1.00 | 40.28 |
| 789 | C | ASN | A | 321 | 56.732 | 8.291 | 79.149 | 1.00 | 30.47 |
| 790 | O | ASN | A | 321 | 57.365 | 7.343 | 78.722 | 1.00 | 30.73 |
| 791 | N | HIS | A | 322 | 55.657 | 8.755 | 78.514 | 1.00 | 34.71 |
| 792 | CA | HIS | A | 322 | 55.162 | 8.076 | 77.324 | 1.00 | 41.59 |
| 793 | CB | HIS | A | 322 | 53.701 | 8.431 | 77.059 | 1.00 | 54.29 |
| 794 | CG | HIS | A | 322 | 53.408 | 9.909 | 76.990 | 1.00 | 76.97 |
| 795 | CD2 | HIS | A | 322 | 53.251 | 10.730 | 75.920 | 1.00 | 80.64 |
| 796 | ND1 | HIS | A | 322 | 53.153 | 10.696 | 78.102 | 1.00 | 85.25 |
| 797 | CE1 | HIS | A | 322 | 52.850 | 11.927 | 77.721 | 1.00 | 83.58 |
| 798 | NE2 | HIS | A | 322 | 52.904 | 11.973 | 76.400 | 1.00 | 84.03 |
| 799 | C | HIS | A | 322 | 55.967 | 8.288 | 76.065 | 1.00 | 43.58 |
| 800 | O | HIS | A | 322 | 56.226 | 7.349 | 75.317 | 1.00 | 44.52 |
| 801 | N | GLU | A | 323 | 56.400 | 9.512 | 75.840 | 1.00 | 44.35 |
| 802 | CA | GLU | A | 323 | 57.174 | 9.799 | 74.656 | 1.00 | 46.93 |
| 803 | CB | GLU | A | 323 | 57.410 | 11.297 | 74.538 | 1.00 | 56.88 |
| 804 | CG | GLU | A | 323 | 57.359 | 12.013 | 75.886 | 1.00 | 75.19 |
| 805 | CD | GLU | A | 323 | 57.518 | 13.517 | 75.759 | 1.00 | 83.55 |
| 806 | OE1 | GLU | A | 323 | 58.552 | 13.965 | 75.194 | 1.00 | 84.32 |
| 807 | OE2 | GLU | A | 323 | 56.604 | 14.250 | 76.212 | 1.00 | 85.25 |
| 808 | C | GLU | A | 323 | 58.494 | 9.064 | 74.667 | 1.00 | 44.24 |
| 809 | O | GLU | A | 323 | 58.897 | 8.550 | 73.625 | 1.00 | 46.47 |
| 810 | N | THR | A | 324 | 59.148 | 8.979 | 75.833 | 1.00 | 36.96 |
| 811 | CA | THR | A | 324 | 60.461 | 8.325 | 75.921 | 1.00 | 31.82 |
| 812 | CB | THR | A | 324 | 61.419 | 9.124 | 76.811 | 1.00 | 28.94 |
| 813 | OG1 | THR | A | 324 | 61.147 | 8.850 | 78.192 | 1.00 | 43.62 |
| 814 | CG2 | THR | A | 324 | 61.247 | 10.617 | 76.565 | 1.00 | 42.78 |
| 815 | C | THR | A | 324 | 60.450 | 6.876 | 76.410 | 1.00 | 32.14 |
| 816 | O | THR | A | 324 | 61.486 | 6.156 | 76.345 | 1.00 | 19.90 |
| 817 | N | GLU | A | 325 | 59.260 | 6.449 | 76.848 | 1.00 | 33.70 |
| 818 | CA | GLU | A | 325 | 59.017 | 5.099 | 77.386 | 1.00 | 33.30 |
| 819 | CB | GLU | A | 325 | 59.101 | 4.047 | 76.270 | 1.00 | 39.05 |
| 820 | CG | GLU | A | 325 | 57.997 | 4.113 | 75.234 | 1.00 | 41.63 |
| 821 | CD | GLU | A | 325 | 57.894 | 2.822 | 74.473 | 1.00 | 48.98 |
| 822 | OE1 | GLU | A | 325 | 57.333 | 1.843 | 75.016 | 1.00 | 67.31 |
| 823 | OE2 | GLU | A | 325 | 58.402 | 2.755 | 73.344 | 1.00 | 53.65 |
| 824 | C | GLU | A | 325 | 59.966 | 4.711 | 78.541 | 1.00 | 29.19 |
| 825 | O | GLU | A | 325 | 60.150 | 3.541 | 78.819 | 1.00 | 29.37 |
| 826 | N | CYS | A | 326 | 60.543 | 5.708 | 79.210 | 1.00 | 24.41 |
| 827 | CA | CYS | A | 326 | 61.463 | 5.464 | 80.303 | 1.00 | 19.33 |
| 828 | CB | CYS | A | 326 | 62.771 | 6.209 | 80.096 | 1.00 | 19.88 |
| 829 | SG | CYS | A | 326 | 63.687 | 5.503 | 78.803 | 1.00 | 24.37 |
| 830 | C | CYS | A | 326 | 60.903 | 5.860 | 81.623 | 1.00 | 15.95 |
| 831 | O | CYS | A | 326 | 60.151 | 6.805 | 81.730 | 1.00 | 14.68 |
| 832 | N | ILE | A | 327 | 61.278 | 5.114 | 82.647 | 1.00 | 16.97 |
| 833 | CA | ILE | A | 327 | 60.772 | 5.412 | 83.970 | 1.00 | 20.52 |
| 834 | CB | ILE | A | 327 | 60.439 | 4.135 | 84.761 | 1.00 | 22.42 |
| 835 | CG2 | ILE | A | 327 | 60.222 | 4.478 | 86.226 | 1.00 | 8.74 |
| 836 | CG1 | ILE | A | 327 | 59.173 | 3.497 | 84.183 | 1.00 | 16.37 |
| 837 | CD1 | ILE | A | 327 | 58.975 | 2.088 | 84.631 | 1.00 | 22.14 |
| 838 | C | ILE | A | 327 | 61.875 | 6.156 | 84.655 | 1.00 | 22.73 |
| 839 | O | ILE | A | 327 | 62.994 | 5.654 | 84.796 | 1.00 | 26.08 |
| 840 | N | THR | A | 328 | 61.584 | 7.373 | 85.062 | 1.00 | 19.96 |
| 841 | CA | THR | A | 328 | 62.618 | 8.139 | 85.713 | 1.00 | 19.11 |
| 842 | CB | THR | A | 328 | 62.740 | 9.541 | 85.056 | 1.00 | 11.47 |
| 843 | OG1 | THR | A | 328 | 63.358 | 10.424 | 85.985 | 1.00 | 12.92 |
| 844 | CG2 | THR | A | 328 | 61.417 | 10.084 | 84.701 | 1.00 | 12.27 |
| 845 | C | THR | A | 328 | 62.366 | 8.236 | 87.245 | 1.00 | 18.38 |
| 846 | O | THR | A | 328 | 61.446 | 8.922 | 87.717 | 1.00 | 14.72 |
| 847 | N | PHE | A | 329 | 63.183 | 7.517 | 88.006 | 1.00 | 16.51 |
| 848 | CA | PHE | A | 329 | 63.082 | 7.513 | 89.443 | 1.00 | 18.38 |
| 849 | CB | PHE | A | 329 | 64.105 | 6.547 | 90.027 | 1.00 | 18.29 |
| 850 | CG | PHE | A | 329 | 64.090 | 5.188 | 89.405 | 1.00 | 19.31 |
| 851 | CD1 | PHE | A | 329 | 63.067 | 4.818 | 88.522 | 1.00 | 13.87 |
| 852 | CD2 | PHE | A | 329 | 65.075 | 4.242 | 89.743 | 1.00 | 16.73 |
| 853 | CE1 | PHE | A | 329 | 63.000 | 3.509 | 87.971 | 1.00 | 7.96 |
| 854 | CE2 | PHE | A | 329 | 65.027 | 2.934 | 89.207 | 1.00 | 9.29 |
| 855 | CZ | PHE | A | 329 | 63.975 | 2.572 | 88.314 | 1.00 | 7.44 |
| 856 | C | PHE | A | 329 | 63.339 | 8.870 | 90.069 | 1.00 | 22.72 |
| 857 | O | PHE | A | 329 | 62.549 | 9.343 | 90.833 | 1.00 | 18.85 |
| 858 | N | LEU | A | 330 | 64.460 | 9.486 | 89.721 | 1.00 | 27.93 |
| 859 | CA | LEU | A | 330 | 64.873 | 10.747 | 90.316 | 1.00 | 30.76 |
| 860 | CB | LEU | A | 330 | 66.148 | 10.534 | 91.130 | 1.00 | 34.14 |
| 861 | CG | LEU | A | 330 | 66.044 | 10.077 | 92.550 | 1.00 | 29.90 |
| 862 | CD1 | LEU | A | 330 | 67.392 | 10.061 | 93.207 | 1.00 | 44.27 |
| 863 | CD2 | LEU | A | 330 | 65.150 | 11.082 | 93.251 | 1.00 | 53.47 |
| 864 | C | LEU | A | 330 | 65.242 | 11.663 | 89.216 | 1.00 | 35.80 |
| 865 | O | LEU | A | 330 | 64.976 | 11.335 | 88.068 | 1.00 | 34.20 |
| 866 | N | LYS | A | 331 | 65.830 | 12.821 | 89.555 | 1.00 | 39.21 |
| 867 | CA | LYS | A | 331 | 66.315 | 13.677 | 88.481 | 1.00 | 40.72 |
| 868 | CB | LYS | A | 331 | 66.344 | 15.172 | 88.848 | 1.00 | 42.67 |
| 869 | CG | LYS | A | 331 | 67.703 | 15.843 | 88.676 | 1.00 | 51.34 |
| 870 | CD | LYS | A | 331 | 67.770 | 16.800 | 87.501 | 1.00 | 53.38 |
| 871 | CE | LYS | A | 331 | 69.185 | 17.325 | 87.365 | 1.00 | 50.95 |
| 872 | NZ | LYS | A | 331 | 69.369 | 18.028 | 86.053 | 1.00 | 59.13 |
| 873 | C | LYS | A | 331 | 67.706 | 13.039 | 88.232 | 1.00 | 38.02 |
| 874 | O | LYS | A | 331 | 68.390 | 12.532 | 89.142 | 1.00 | 38.56 |
| 875 | N | ASP | A | 332 | 68.088 | 13.082 | 86.972 | 1.00 | 36.23 |
| 876 | CA | ASP | A | 332 | 69.246 | 12.404 | 86.422 | 1.00 | 35.75 |
| 877 | CB | ASP | A | 332 | 70.597 | 13.264 | 86.339 | 1.00 | 36.31 |
| 878 | CG | ASP | A | 332 | 71.188 | 13.706 | 87.656 | 1.00 | 43.37 |
| 879 | OD1 | ASP | A | 332 | 72.125 | 13.010 | 88.122 | 1.00 | 55.31 |
| 880 | OD2 | ASP | A | 332 | 70.770 | 14.763 | 88.198 | 1.00 | 45.32 |
| 881 | C | ASP | A | 332 | 69.377 | 10.932 | 86.923 | 1.00 | 32.13 |
| 882 | O | ASP | A | 332 | 70.442 | 10.431 | 87.220 | 1.00 | 33.64 |
| 883 | N | PHE | A | 333 | 68.241 | 10.219 | 86.921 | 1.00 | 30.11 |
| 884 | CA | PHE | A | 333 | 68.083 | 8.766 | 87.334 | 1.00 | 30.48 |
| 885 | CB | PHE | A | 333 | 67.696 | 8.577 | 88.831 | 1.00 | 19.29 |
| 886 | CG | PHE | A | 333 | 68.863 | 8.544 | 89.783 | 1.00 | 21.29 |
| 887 | CD1 | PHE | A | 333 | 69.532 | 7.343 | 90.010 | 1.00 | 16.07 |
| 888 | CD2 | PHE | A | 333 | 69.302 | 9.722 | 90.446 | 1.00 | 12.63 |
| 889 | CE1 | PHE | A | 333 | 70.632 | 7.289 | 90.878 | 1.00 | 16.55 |
| 890 | CE2 | PHE | A | 333 | 70.384 | 9.699 | 91.310 | 1.00 | 16.16 |
| 891 | CZ | PHE | A | 333 | 71.064 | 8.476 | 91.536 | 1.00 | 16.98 |
| 892 | C | PHE | A | 333 | 66.920 | 8.119 | 86.552 | 1.00 | 28.27 |
| 893 | O | PHE | A | 333 | 65.862 | 7.807 | 87.135 | 1.00 | 33.02 |
| 894 | N | THR | A | 334 | 67.139 | 7.860 | 85.270 | 1.00 | 23.48 |
| 895 | CA | THR | A | 334 | 66.115 | 7.295 | 84.392 | 1.00 | 24.00 |
| 896 | CB | THR | A | 334 | 65.828 | 8.337 | 83.276 | 1.00 | 20.94 |
| 897 | OG1 | THR | A | 334 | 65.314 | 9.524 | 83.895 | 1.00 | 18.52 |
| 898 | CG2 | THR | A | 334 | 64.816 | 7.839 | 82.271 | 1.00 | 17.24 |
| 899 | C | THR | A | 334 | 66.448 | 5.906 | 83.803 | 1.00 | 21.68 |
| 900 | O | THR | A | 334 | 67.595 | 5.599 | 83.563 | 1.00 | 20.97 |
| 901 | N | TYR | A | 335 | 65.442 | 5.071 | 83.581 | 1.00 | 18.64 |
| 902 | CA | TYR | A | 335 | 65.697 | 3.741 | 83.045 | 1.00 | 19.91 |
| 903 | CB | TYR | A | 335 | 65.763 | 2.728 | 84.203 | 1.00 | 18.15 |
| 904 | CG | TYR | A | 335 | 66.664 | 3.239 | 85.335 | 1.00 | 19.69 |
| 905 | CD1 | TYR | A | 335 | 68.052 | 3.132 | 85.244 | 1.00 | 9.91 |
| 906 | CE1 | TYR | A | 335 | 68.888 | 3.773 | 86.128 | 1.00 | 16.03 |
| 907 | CD2 | TYR | A | 335 | 66.134 | 3.993 | 86.382 | 1.00 | 12.55 |
| 908 | CE2 | TYR | A | 335 | 66.948 | 4.638 | 87.264 | 1.00 | 21.32 |
| 909 | CZ | TYR | A | 335 | 68.331 | 4.543 | 87.130 | 1.00 | 22.31 |
| 910 | OH | TYR | A | 335 | 69.130 | 5.334 | 87.921 | 1.00 | 14.35 |
| 911 | C | TYR | A | 335 | 64.668 | 3.320 | 81.995 | 1.00 | 21.65 |
| 912 | O | TYR | A | 335 | 63.458 | 3.601 | 82.094 | 1.00 | 25.19 |
| 913 | N | SER | A | 336 | 65.188 | 2.659 | 80.972 | 1.00 | 21.19 |
| 914 | CA | SER | A | 336 | 64.408 | 2.168 | 79.852 | 1.00 | 19.61 |
| 915 | CB | SER | A | 336 | 65.292 | 2.113 | 78.639 | 1.00 | 15.94 |
| 916 | OG | SER | A | 336 | 66.198 | 1.026 | 78.795 | 1.00 | 17.24 |
| 917 | C | SER | A | 336 | 63.993 | 0.751 | 80.156 | 1.00 | 22.31 |
| 918 | O | SER | A | 336 | 64.309 | 0.216 | 81.203 | 1.00 | 22.32 |
| 919 | N | LYS | A | 337 | 63.308 | 0.114 | 79.224 | 1.00 | 27.92 |
| 920 | CA | LYS | A | 337 | 62.927 | −1.267 | 79.471 | 1.00 | 32.61 |
| 921 | CB | LYS | A | 337 | 61.992 | −1.794 | 78.378 | 1.00 | 38.84 |
| 922 | CG | LYS | A | 337 | 60.512 | −1.671 | 78.689 | 1.00 | 45.22 |
| 923 | CD | LYS | A | 337 | 59.685 | −2.866 | 78.099 | 1.00 | 37.58 |
| 924 | CE | LYS | A | 337 | 58.528 | −2.372 | 77.209 | 1.00 | 33.90 |
| 925 | NZ | LYS | A | 337 | 57.646 | −3.499 | 76.797 | 1.00 | 26.57 |
| 926 | C | LYS | A | 337 | 64.220 | −2.089 | 79.447 | 1.00 | 32.43 |
| 927 | O | LYS | A | 337 | 64.463 | −2.938 | 80.297 | 1.00 | 31.18 |
| 928 | N | ASP | A | 338 | 65.062 | −1.826 | 78.459 | 1.00 | 34.81 |
| 929 | CA | ASP | A | 338 | 66.281 | −2.587 | 78.357 | 1.00 | 33.25 |
| 930 | CB | ASP | A | 338 | 67.003 | −2.294 | 77.048 | 1.00 | 31.70 |
| 931 | CG | ASP | A | 338 | 68.258 | −3.122 | 76.918 | 1.00 | 46.29 |
| 932 | OD1 | ASP | A | 338 | 68.187 | −4.373 | 77.017 | 1.00 | 52.08 |
| 933 | OD2 | ASP | A | 338 | 69.338 | −2.540 | 76.732 | 1.00 | 52.04 |
| 934 | C | ASP | A | 338 | 67.200 | −2.355 | 79.561 | 1.00 | 31.99 |
| 935 | O | ASP | A | 338 | 68.063 | −3.187 | 79.872 | 1.00 | 32.73 |
| 936 | N | ASP | A | 339 | 67.027 | −1.240 | 80.260 | 1.00 | 29.20 |
| 937 | CA | ASP | A | 339 | 67.854 | −1.030 | 81.439 | 1.00 | 26.90 |
| 938 | CB | ASP | A | 339 | 67.660 | 0.380 | 81.977 | 1.00 | 28.69 |
| 939 | CG | ASP | A | 339 | 68.254 | 1.424 | 81.060 | 1.00 | 29.23 |
| 940 | OD1 | ASP | A | 339 | 69.450 | 1.260 | 80.684 | 1.00 | 24.71 |
| 941 | OD2 | ASP | A | 339 | 67.534 | 2.408 | 80.719 | 1.00 | 32.90 |
| 942 | C | ASP | A | 339 | 67.492 | −2.070 | 82.498 | 1.00 | 26.21 |
| 943 | O | ASP | A | 339 | 68.363 | −2.587 | 83.209 | 1.00 | 29.03 |
| 944 | N | PHE | A | 340 | 66.196 | −2.374 | 82.568 | 1.00 | 27.72 |
| 945 | CA | PHE | A | 340 | 65.616 | −3.365 | 83.475 | 1.00 | 26.22 |
| 946 | CB | PHE | A | 340 | 64.096 | −3.345 | 83.346 | 1.00 | 28.71 |
| 947 | CG | PHE | A | 340 | 63.452 | −2.309 | 84.162 | 1.00 | 26.69 |
| 948 | CD1 | PHE | A | 340 | 62.458 | −2.656 | 85.044 | 1.00 | 24.75 |
| 949 | CD2 | PHE | A | 340 | 63.870 | −0.989 | 84.092 | 1.00 | 29.16 |
| 950 | CE1 | PHE | A | 340 | 61.884 | −1.703 | 85.865 | 1.00 | 16.74 |
| 951 | CE2 | PHE | A | 340 | 63.312 | −0.041 | 84.896 | 1.00 | 22.67 |
| 952 | CZ | PHE | A | 340 | 62.315 | −0.400 | 85.789 | 1.00 | 25.21 |
| 953 | C | PHE | A | 340 | 66.115 | −4.746 | 83.098 | 1.00 | 30.07 |
| 954 | O | PHE | A | 340 | 66.430 | −5.561 | 83.970 | 1.00 | 32.54 |
| 955 | N | HIS | A | 341 | 66.178 | −5.020 | 81.794 | 1.00 | 29.06 |
| 956 | CA | HIS | A | 341 | 66.667 | −6.307 | 81.388 | 1.00 | 33.09 |
| 957 | CB | HIS | A | 341 | 66.523 | −6.501 | 79.894 | 1.00 | 41.29 |
| 958 | CG | HIS | A | 341 | 66.697 | −7.923 | 79.493 | 1.00 | 51.69 |
| 959 | CD2 | HIS | A | 341 | 66.108 | −9.044 | 79.972 | 1.00 | 62.95 |
| 960 | ND1 | HIS | A | 341 | 67.639 | −8.343 | 78.579 | 1.00 | 60.58 |
| 961 | CE1 | HIS | A | 341 | 67.626 | −9.664 | 78.516 | 1.00 | 67.99 |
| 962 | NE2 | HIS | A | 341 | 66.706 | −10.113 | 79.355 | 1.00 | 74.79 |
| 963 | C | HIS | A | 341 | 68.131 | −6.416 | 81.781 | 1.00 | 30.10 |
| 964 | O | HIS | A | 341 | 68.564 | −7.399 | 82.397 | 1.00 | 33.29 |
| 965 | N | ARG | A | 342 | 68.885 | −5.383 | 81.435 | 1.00 | 26.27 |
| 966 | CA | ARG | A | 342 | 70.297 | −5.350 | 81.751 | 1.00 | 27.76 |
| 967 | CB | ARG | A | 342 | 70.910 | −4.034 | 81.307 | 1.00 | 21.84 |
| 968 | CG | ARG | A | 342 | 71.231 | −3.988 | 79.842 | 1.00 | 33.15 |
| 969 | CD | ARG | A | 342 | 72.195 | −2.884 | 79.494 | 1.00 | 37.97 |
| 970 | NE | ARG | A | 342 | 71.933 | −2.375 | 78.148 | 1.00 | 56.61 |
| 971 | CZ | ARG | A | 342 | 71.144 | −1.332 | 77.893 | 1.00 | 57.88 |
| 972 | NH1 | ARG | A | 342 | 70.552 | −0.694 | 78.890 | 1.00 | 63.90 |
| 973 | NH2 | ARG | A | 342 | 70.934 | −0.931 | 76.643 | 1.00 | 54.40 |
| 974 | C | ARG | A | 342 | 70.472 | −5.505 | 83.246 | 1.00 | 32.21 |
| 975 | O | ARG | A | 342 | 71.322 | −6.289 | 83.719 | 1.00 | 37.76 |
| 976 | N | ALA | A | 343 | 69.652 | −4.761 | 83.989 | 1.00 | 29.12 |
| 977 | CA | ALA | A | 343 | 69.688 | −4.793 | 85.445 | 1.00 | 26.57 |
| 978 | CB | ALA | A | 343 | 68.664 | −3.819 | 85.987 | 1.00 | 24.89 |
| 979 | C | ALA | A | 343 | 69.483 | −6.202 | 86.064 | 1.00 | 26.96 |
| 980 | O | ALA | A | 343 | 69.650 | −6.372 | 87.271 | 1.00 | 25.29 |
| 981 | N | GLY | A | 344 | 69.106 | −7.188 | 85.246 | 1.00 | 24.87 |
| 982 | CA | GLY | A | 344 | 68.944 | −8.544 | 85.743 | 1.00 | 23.67 |
| 983 | C | GLY | A | 344 | 67.535 | −9.089 | 85.737 | 1.00 | 27.67 |
| 984 | O | GLY | A | 344 | 67.341 | −10.318 | 85.803 | 1.00 | 25.72 |
| 985 | N | LEU | A | 345 | 66.551 | −8.188 | 85.650 | 1.00 | 27.49 |
| 986 | CA | LEU | A | 345 | 65.135 | −8.578 | 85.670 | 1.00 | 24.58 |
| 987 | CB | LEU | A | 345 | 64.258 | −7.342 | 85.811 | 1.00 | 23.55 |
| 988 | CG | LEU | A | 345 | 64.619 | −6.367 | 86.941 | 1.00 | 15.50 |
| 989 | CD1 | LEU | A | 345 | 63.839 | −5.038 | 86.748 | 1.00 | 19.24 |
| 990 | CD2 | LEU | A | 345 | 64.305 | −6.978 | 88.234 | 1.00 | 20.63 |
| 991 | C | LEU | A | 345 | 64.671 | −9.347 | 84.453 | 1.00 | 23.25 |
| 992 | O | LEU | A | 345 | 65.177 | −9.156 | 83.368 | 1.00 | 23.62 |
| 993 | N | GLN | A | 346 | 63.695 | −10.218 | 84.663 | 1.00 | 23.03 |
| 994 | CA | GLN | A | 346 | 63.075 | −11.031 | 83.605 | 1.00 | 23.05 |
| 995 | CB | GLN | A | 346 | 62.269 | −12.178 | 84.208 | 1.00 | 21.66 |
| 996 | CG | GLN | A | 346 | 63.072 | −13.195 | 84.943 | 1.00 | 40.94 |
| 997 | CD | GLN | A | 346 | 62.269 | −14.431 | 85.340 | 1.00 | 38.02 |
| 998 | OE1 | GLN | A | 346 | 61.264 | −14.330 | 86.050 | 1.00 | 26.11 |
| 999 | NE2 | GLN | A | 346 | 62.719 | −15.605 | 84.883 | 1.00 | 38.61 |
| 1000 | C | GLN | A | 346 | 62.064 | −10.237 | 82.740 | 1.00 | 24.22 |
| 1001 | O | GLN | A | 346 | 61.293 | −9.377 | 83.240 | 1.00 | 13.82 |
| 1002 | N | VAL | A | 347 | 62.057 | −10.566 | 81.447 | 1.00 | 25.45 |
| 1003 | CA | VAL | A | 347 | 61.136 | −9.952 | 80.483 | 1.00 | 24.56 |
| 1004 | CB | VAL | A | 347 | 61.344 | −10.578 | 79.024 | 1.00 | 23.19 |
| 1005 | CG1 | VAL | A | 347 | 60.788 | −11.989 | 78.959 | 1.00 | 21.34 |
| 1006 | CG2 | VAL | A | 347 | 60.655 | −9.760 | 77.970 | 1.00 | 10.36 |
| 1007 | C | VAL | A | 347 | 59.705 | −10.260 | 80.989 | 1.00 | 26.68 |
| 1008 | O | VAL | A | 347 | 58.768 | −9.520 | 80.752 | 1.00 | 28.59 |
| 1009 | N | GLU | A | 348 | 59.562 | −11.370 | 81.700 | 1.00 | 28.04 |
| 1010 | CA | GLU | A | 348 | 58.280 | −11.800 | 82.242 | 1.00 | 26.61 |
| 1011 | CB | GLU | A | 348 | 58.448 | −13.098 | 83.028 | 1.00 | 31.77 |
| 1012 | CG | GLU | A | 348 | 59.539 | −14.015 | 82.550 | 1.00 | 33.06 |
| 1013 | CD | GLU | A | 348 | 59.186 | −14.628 | 81.283 | 1.00 | 37.73 |
| 1014 | OE1 | GLU | A | 348 | 58.072 | −15.170 | 81.211 | 1.00 | 45.41 |
| 1015 | OE2 | GLU | A | 348 | 60.011 | −14.568 | 80.354 | 1.00 | 60.92 |
| 1016 | C | GLU | A | 348 | 57.744 | −10.761 | 83.200 | 1.00 | 23.96 |
| 1017 | O | GLU | A | 348 | 56.551 | −10.584 | 83.297 | 1.00 | 24.06 |
| 1018 | N | PHE | A | 349 | 58.646 | −10.119 | 83.932 | 1.00 | 22.08 |
| 1019 | CA | PHE | A | 349 | 58.295 | −9.085 | 84.910 | 1.00 | 24.53 |
| 1020 | CB | PHE | A | 349 | 59.303 | −9.138 | 86.082 | 1.00 | 14.36 |
| 1021 | CG | PHE | A | 349 | 58.951 | −8.259 | 87.217 | 1.00 | 9.71 |
| 1022 | CD1 | PHE | A | 349 | 57.638 | −8.190 | 87.673 | 1.00 | 16.13 |
| 1023 | CD2 | PHE | A | 349 | 59.932 | −7.572 | 87.908 | 1.00 | 14.92 |
| 1024 | CE1 | PHE | A | 349 | 57.301 | −7.453 | 88.820 | 1.00 | 5.25 |
| 1025 | CE2 | PHE | A | 349 | 59.609 | −6.823 | 89.075 | 1.00 | 13.59 |
| 1026 | CZ | PHE | A | 349 | 58.284 | −6.775 | 89.524 | 1.00 | 7.02 |
| 1027 | C | PHE | A | 349 | 58.256 | −7.659 | 84.283 | 1.00 | 25.25 |
| 1028 | O | PHE | A | 349 | 57.298 | −6.926 | 84.448 | 1.00 | 23.80 |
| 1029 | N | ILE | A | 350 | 59.313 | −7.294 | 83.565 | 1.00 | 25.82 |
| 1030 | CA | ILE | A | 350 | 59.458 | −6.005 | 82.885 | 1.00 | 22.66 |
| 1031 | CB | ILE | A | 350 | 60.763 | −6.071 | 82.038 | 1.00 | 22.42 |
| 1032 | CG2 | ILE | A | 350 | 60.996 | −4.796 | 81.233 | 1.00 | 14.30 |
| 1033 | CG1 | ILE | A | 350 | 61.922 | −6.374 | 82.962 | 1.00 | 10.32 |
| 1034 | CD1 | ILE | A | 350 | 63.165 | −6.731 | 82.164 | 1.00 | 13.28 |
| 1035 | C | ILE | A | 350 | 58.252 | −5.582 | 81.982 | 1.00 | 26.39 |
| 1036 | O | ILE | A | 350 | 57.647 | −4.504 | 82.146 | 1.00 | 19.95 |
| 1037 | N | ASN | A | 351 | 57.899 | −6.418 | 81.012 | 1.00 | 27.99 |
| 1038 | CA | ASN | A | 351 | 56.769 | −6.070 | 80.143 | 1.00 | 26.87 |
| 1039 | CB | ASN | A | 351 | 56.474 | −7.190 | 79.134 | 1.00 | 20.91 |
| 1040 | CG | ASN | A | 351 | 57.688 | −7.516 | 78.253 | 1.00 | 26.68 |
| 1041 | OD1 | ASN | A | 351 | 58.301 | −6.645 | 77.665 | 1.00 | 21.67 |
| 1042 | ND2 | ASN | A | 351 | 58.030 | −8.781 | 78.174 | 1.00 | 41.74 |
| 1043 | C | ASN | A | 351 | 55.499 | −5.714 | 80.925 | 1.00 | 20.74 |
| 1044 | O | ASN | A | 351 | 54.935 | −4.656 | 80.698 | 1.00 | 23.26 |
| 1045 | N | PRO | A | 352 | 55.043 | −6.580 | 81.855 | 1.00 | 18.21 |
| 1046 | CD | PRO | A | 352 | 55.479 | −7.970 | 82.083 | 1.00 | 12.88 |
| 1047 | CA | PRO | A | 352 | 53.830 | −6.290 | 82.639 | 1.00 | 17.05 |
| 1048 | CB | PRO | A | 352 | 53.740 | −7.479 | 83.592 | 1.00 | 13.45 |
| 1049 | CG | PRO | A | 352 | 54.232 | −8.584 | 82.780 | 1.00 | 5.25 |
| 1050 | C | PRO | A | 352 | 53.872 | −4.943 | 83.386 | 1.00 | 18.19 |
| 1051 | O | PRO | A | 352 | 52.828 | −4.249 | 83.490 | 1.00 | 12.42 |
| 1052 | N | ILE | A | 353 | 55.059 | −4.553 | 83.879 | 1.00 | 17.57 |
| 1053 | CA | ILE | A | 353 | 55.125 | −3.280 | 84.574 | 1.00 | 17.31 |
| 1054 | CB | ILE | A | 353 | 56.360 | −3.127 | 85.516 | 1.00 | 14.06 |
| 1055 | CG2 | ILE | A | 353 | 57.262 | −4.287 | 85.470 | 1.00 | 20.72 |
| 1056 | CG1 | ILE | A | 353 | 57.037 | −1.816 | 85.222 | 1.00 | 16.47 |
| 1057 | CD1 | ILE | A | 353 | 58.349 | −1.669 | 85.959 | 1.00 | 30.09 |
| 1058 | C | ILE | A | 353 | 55.043 | −2.100 | 83.608 | 1.00 | 16.16 |
| 1059 | O | ILE | A | 353 | 54.418 | −1.061 | 83.893 | 1.00 | 19.52 |
| 1060 | N | PHE | A | 354 | 55.623 | −2.260 | 82.437 | 1.00 | 17.20 |
| 1061 | CA | PHE | A | 354 | 55.541 | −1.161 | 81.496 | 1.00 | 16.87 |
| 1062 | CB | PHE | A | 354 | 56.611 | −1.296 | 80.428 | 1.00 | 15.75 |
| 1063 | CG | PHE | A | 354 | 57.961 | −0.845 | 80.907 | 1.00 | 26.72 |
| 1064 | CD1 | PHE | A | 354 | 58.624 | −1.554 | 81.933 | 1.00 | 26.43 |
| 1065 | CD2 | PHE | A | 354 | 58.542 | 0.320 | 80.404 | 1.00 | 14.53 |
| 1066 | CE1 | PHE | A | 354 | 59.839 | −1.104 | 82.452 | 1.00 | 15.82 |
| 1067 | CE2 | PHE | A | 354 | 59.759 | 0.781 | 80.915 | 1.00 | 20.30 |
| 1068 | CZ | PHE | A | 354 | 60.410 | 0.081 | 81.934 | 1.00 | 23.97 |
| 1069 | C | PHE | A | 354 | 54.164 | −1.113 | 80.892 | 1.00 | 16.64 |
| 1070 | O | PHE | A | 354 | 53.646 | −0.048 | 80.543 | 1.00 | 15.01 |
| 1071 | N | GLU | A | 355 | 53.538 | −2.275 | 80.819 | 1.00 | 17.82 |
| 1072 | CA | GLU | A | 355 | 52.205 | −2.313 | 80.279 | 1.00 | 24.65 |
| 1073 | CB | GLU | A | 355 | 51.784 | −3.751 | 79.975 | 1.00 | 27.67 |
| 1074 | CG | GLU | A | 355 | 51.021 | −3.888 | 78.666 | 1.00 | 54.83 |
| 1075 | CD | GLU | A | 355 | 50.285 | −2.598 | 78.291 | 1.00 | 73.11 |
| 1076 | OE1 | GLU | A | 355 | 50.069 | −2.330 | 77.083 | 1.00 | 70.34 |
| 1077 | OE2 | GLU | A | 355 | 49.912 | −1.833 | 79.206 | 1.00 | 84.30 |
| 1078 | C | GLU | A | 355 | 51.276 | −1.684 | 81.325 | 1.00 | 25.67 |
| 1079 | O | GLU | A | 355 | 50.267 | −1.008 | 81.025 | 1.00 | 25.52 |
| 1080 | N | PHE | A | 356 | 51.637 | −1.878 | 82.580 | 1.00 | 24.31 |
| 1081 | CA | PHE | A | 356 | 50.807 | −1.331 | 83.628 | 1.00 | 21.24 |
| 1082 | CB | PHE | A | 356 | 51.186 | −1.932 | 84.954 | 1.00 | 16.93 |
| 1083 | CG | PHE | A | 356 | 50.490 | −1.315 | 86.093 | 1.00 | 9.37 |
| 1084 | CD1 | PHE | A | 356 | 49.216 | −1.697 | 86.425 | 1.00 | 11.26 |
| 1085 | CD2 | PHE | A | 356 | 51.113 | −0.325 | 86.834 | 1.00 | 12.62 |
| 1086 | CE1 | PHE | A | 356 | 48.560 | −1.097 | 87.490 | 1.00 | 5.27 |
| 1087 | CE2 | PHE | A | 356 | 50.459 | 0.285 | 87.901 | 1.00 | 6.44 |
| 1088 | CZ | PHE | A | 356 | 49.187 | −0.104 | 88.225 | 1.00 | 6.39 |
| 1089 | C | PHE | A | 356 | 50.980 | 0.152 | 83.699 | 1.00 | 18.18 |
| 1090 | O | PHE | A | 356 | 50.013 | 0.863 | 84.023 | 1.00 | 12.84 |
| 1091 | N | SER | A | 357 | 52.210 | 0.606 | 83.419 | 1.00 | 18.00 |
| 1092 | CA | SER | A | 357 | 52.527 | 2.037 | 83.442 | 1.00 | 20.63 |
| 1093 | CB | SER | A | 357 | 54.005 | 2.263 | 83.194 | 1.00 | 24.68 |
| 1094 | OG | SER | A | 357 | 54.765 | 1.848 | 84.302 | 1.00 | 28.70 |
| 1095 | C | SER | A | 357 | 51.721 | 2.819 | 82.408 | 1.00 | 20.09 |
| 1096 | O | SER | A | 357 | 51.288 | 3.946 | 82.673 | 1.00 | 24.68 |
| 1097 | N | ARG | A | 358 | 51.493 | 2.208 | 81.242 | 1.00 | 21.71 |
| 1098 | CA | ARG | A | 358 | 50.709 | 2.849 | 80.169 | 1.00 | 20.24 |
| 1099 | CB | ARG | A | 358 | 50.932 | 2.135 | 78.816 | 1.00 | 21.60 |
| 1100 | CG | ARG | A | 358 | 52.392 | 2.178 | 78.364 | 1.00 | 24.91 |
| 1101 | CD | ARG | A | 358 | 52.695 | 1.483 | 77.018 | 1.00 | 22.98 |
| 1102 | NE | ARG | A | 358 | 53.676 | 0.374 | 77.211 | 1.00 | 49.01 |
| 1103 | CZ | ARG | A | 358 | 53.385 | −0.942 | 77.152 | 1.00 | 46.01 |
| 1104 | NH1 | ARG | A | 358 | 52.145 | −1.326 | 76.877 | 1.00 | 36.77 |
| 1105 | NH2 | ARG | A | 358 | 54.306 | −1.879 | 77.438 | 1.00 | 30.95 |
| 1106 | C | ARG | A | 358 | 49.240 | 2.855 | 80.533 | 1.00 | 16.71 |
| 1107 | O | ARG | A | 358 | 48.528 | 3.825 | 80.293 | 1.00 | 16.15 |
| 1108 | N | ALA | A | 359 | 48.789 | 1.756 | 81.111 | 1.00 | 15.42 |
| 1109 | CA | ALA | A | 359 | 47.401 | 1.699 | 81.547 | 1.00 | 18.48 |
| 1110 | CB | ALA | A | 359 | 47.100 | 0.379 | 82.201 | 1.00 | 23.61 |
| 1111 | C | ALA | A | 359 | 47.197 | 2.844 | 82.535 | 1.00 | 17.14 |
| 1112 | O | ALA | A | 359 | 46.214 | 3.581 | 82.437 | 1.00 | 18.07 |
| 1113 | N | MET | A | 360 | 48.147 | 3.008 | 83.455 | 1.00 | 14.89 |
| 1114 | CA | MET | A | 360 | 48.054 | 4.064 | 84.433 | 1.00 | 17.42 |
| 1115 | CB | MET | A | 360 | 49.222 | 3.999 | 85.432 | 1.00 | 17.61 |
| 1116 | CG | MET | A | 360 | 49.061 | 2.921 | 86.502 | 1.00 | 7.42 |
| 1117 | SD | MET | A | 360 | 47.353 | 2.918 | 87.350 | 1.00 | 12.36 |
| 1118 | CE | MET | A | 360 | 47.562 | 3.990 | 88.630 | 1.00 | 10.71 |
| 1119 | C | MET | A | 360 | 47.964 | 5.455 | 83.824 | 1.00 | 18.28 |
| 1120 | O | MET | A | 360 | 47.105 | 6.239 | 84.223 | 1.00 | 20.28 |
| 1121 | N | ARG | A | 361 | 48.827 | 5.791 | 82.867 | 1.00 | 23.50 |
| 1122 | CA | ARG | A | 361 | 48.727 | 7.123 | 82.279 | 1.00 | 27.99 |
| 1123 | CB | ARG | A | 361 | 49.824 | 7.340 | 81.304 | 1.00 | 24.17 |
| 1124 | CG | ARG | A | 361 | 49.598 | 8.583 | 80.524 | 1.00 | 44.39 |
| 1125 | CD | ARG | A | 361 | 50.186 | 8.387 | 79.148 | 1.00 | 59.94 |
| 1126 | NE | ARG | A | 361 | 49.637 | 9.089 | 77.971 | 1.00 | 68.19 |
| 1127 | CZ | ARG | A | 361 | 48.781 | 8.514 | 77.124 | 1.00 | 77.06 |
| 1128 | NH1 | ARG | A | 361 | 48.328 | 7.287 | 77.353 | 1.00 | 83.36 |
| 1129 | NH2 | ARG | A | 361 | 48.565 | 9.036 | 75.926 | 1.00 | 80.65 |
| 1130 | C | ARG | A | 361 | 47.403 | 7.323 | 81.554 | 1.00 | 33.07 |
| 1131 | O | ARG | A | 361 | 46.895 | 8.439 | 81.468 | 1.00 | 35.79 |
| 1132 | N | ARG | A | 362 | 46.845 | 6.227 | 81.040 | 1.00 | 36.68 |
| 1133 | CA | ARG | A | 362 | 45.581 | 6.257 | 80.331 | 1.00 | 35.83 |
| 1134 | CB | ARG | A | 362 | 45.220 | 4.855 | 79.858 | 1.00 | 35.29 |
| 1135 | CG | ARG | A | 362 | 45.220 | 4.707 | 78.335 | 1.00 | 44.87 |
| 1136 | CD | ARG | A | 362 | 46.405 | 3.842 | 77.844 | 1.00 | 40.38 |
| 1137 | NE | ARG | A | 362 | 46.373 | 2.547 | 78.520 | 1.00 | 42.89 |
| 1138 | CZ | ARG | A | 362 | 45.455 | 1.598 | 78.326 | 1.00 | 26.47 |
| 1139 | NH1 | ARG | A | 362 | 44.502 | 1.775 | 77.469 | 1.00 | 39.65 |
| 1140 | NH2 | ARG | A | 362 | 45.461 | 0.473 | 79.017 | 1.00 | 26.69 |
| 1141 | C | ARG | A | 362 | 44.457 | 6.806 | 81.203 | 1.00 | 36.78 |
| 1142 | O | ARG | A | 362 | 43.420 | 7.264 | 80.692 | 1.00 | 44.96 |
| 1143 | N | LEU | A | 363 | 44.650 | 6.764 | 82.516 | 1.00 | 29.80 |
| 1144 | CA | LEU | A | 363 | 43.626 | 7.256 | 83.418 | 1.00 | 20.73 |
| 1145 | CB | LEU | A | 363 | 43.556 | 6.345 | 84.644 | 1.00 | 21.21 |
| 1146 | CG | LEU | A | 363 | 43.163 | 4.913 | 84.314 | 1.00 | 16.69 |
| 1147 | CD1 | LEU | A | 363 | 42.805 | 4.215 | 85.616 | 1.00 | 28.55 |
| 1148 | CD2 | LEU | A | 363 | 41.984 | 4.911 | 83.364 | 1.00 | 15.22 |
| 1149 | C | LEU | A | 363 | 43.849 | 8.721 | 83.831 | 1.00 | 15.12 |
| 1150 | O | LEU | A | 363 | 42.945 | 9.375 | 84.411 | 1.00 | 6.97 |
| 1151 | N | GLY | A | 364 | 45.051 | 9.218 | 83.534 | 1.00 | 12.82 |
| 1152 | CA | GLY | A | 364 | 45.398 | 10.597 | 83.845 | 1.00 | 16.60 |
| 1153 | C | GLY | A | 364 | 45.244 | 11.000 | 85.292 | 1.00 | 20.61 |
| 1154 | O | GLY | A | 364 | 44.613 | 12.037 | 85.593 | 1.00 | 17.13 |
| 1155 | N | LEU | A | 365 | 45.832 | 10.187 | 86.176 | 1.00 | 17.04 |
| 1156 | CA | LEU | A | 365 | 45.768 | 10.460 | 87.593 | 1.00 | 18.84 |
| 1157 | CB | LEU | A | 365 | 46.081 | 9.182 | 88.396 | 1.00 | 27.56 |
| 1158 | CG | LEU | A | 365 | 45.349 | 7.845 | 88.116 | 1.00 | 31.11 |
| 1159 | CD1 | LEU | A | 365 | 45.398 | 6.989 | 89.364 | 1.00 | 30.39 |
| 1160 | CD2 | LEU | A | 365 | 43.901 | 8.072 | 87.747 | 1.00 | 34.95 |
| 1161 | C | LEU | A | 365 | 46.730 | 11.560 | 88.023 | 1.00 | 18.09 |
| 1162 | O | LEU | A | 365 | 47.822 | 11.706 | 87.504 | 1.00 | 15.59 |
| 1163 | N | ASP | A | 366 | 46.331 | 12.335 | 89.017 | 1.00 | 20.32 |
| 1164 | CA | ASP | A | 366 | 47.214 | 13.390 | 89.529 | 1.00 | 20.40 |
| 1165 | CB | ASP | A | 366 | 46.470 | 14.701 | 89.665 | 1.00 | 16.78 |
| 1166 | CG | ASP | A | 366 | 45.278 | 14.567 | 90.521 | 1.00 | 23.28 |
| 1167 | OD1 | ASP | A | 366 | 45.399 | 13.745 | 91.473 | 1.00 | 15.63 |
| 1168 | OD2 | ASP | A | 366 | 44.258 | 15.279 | 90.245 | 1.00 | 18.63 |
| 1169 | C | ASP | A | 366 | 47.788 | 13.010 | 90.893 | 1.00 | 22.44 |
| 1170 | O | ASP | A | 366 | 47.699 | 11.858 | 91.347 | 1.00 | 22.65 |
| 1171 | N | ASP | A | 367 | 48.350 | 13.990 | 91.570 | 1.00 | 21.06 |
| 1172 | CA | ASP | A | 367 | 48.991 | 13.728 | 92.845 | 1.00 | 19.97 |
| 1173 | CB | ASP | A | 367 | 49.820 | 14.962 | 93.171 | 1.00 | 23.70 |
| 1174 | CG | ASP | A | 367 | 50.605 | 15.436 | 91.953 | 1.00 | 33.51 |
| 1175 | OD1 | ASP | A | 367 | 51.760 | 14.973 | 91.788 | 1.00 | 32.01 |
| 1176 | OD2 | ASP | A | 367 | 50.043 | 16.231 | 91.133 | 1.00 | 42.88 |
| 1177 | C | ASP | A | 367 | 48.033 | 13.316 | 93.991 | 1.00 | 18.37 |
| 1178 | O | ASP | A | 367 | 48.382 | 12.443 | 94.831 | 1.00 | 13.39 |
| 1179 | N | ALA | A | 368 | 46.812 | 13.870 | 93.969 | 1.00 | 14.31 |
| 1180 | CA | ALA | A | 368 | 45.829 | 13.569 | 95.010 | 1.00 | 10.24 |
| 1181 | CB | ALA | A | 368 | 44.677 | 14.566 | 94.991 | 1.00 | 5.25 |
| 1182 | C | ALA | A | 368 | 45.278 | 12.183 | 94.839 | 1.00 | 12.43 |
| 1183 | O | ALA | A | 368 | 45.058 | 11.445 | 95.798 | 1.00 | 12.82 |
| 1184 | N | GLU | A | 369 | 45.035 | 11.825 | 93.596 | 1.00 | 13.07 |
| 1185 | CA | GLU | A | 369 | 44.465 | 10.518 | 93.299 | 1.00 | 10.95 |
| 1186 | CB | GLU | A | 369 | 43.991 | 10.490 | 91.852 | 1.00 | 10.95 |
| 1187 | CG | GLU | A | 369 | 43.173 | 11.761 | 91.522 | 1.00 | 22.49 |
| 1188 | CD | GLU | A | 369 | 42.698 | 11.845 | 90.103 | 1.00 | 22.08 |
| 1189 | OE1 | GLU | A | 369 | 43.571 | 11.824 | 89.189 | 1.00 | 15.56 |
| 1190 | OE2 | GLU | A | 369 | 41.452 | 11.923 | 89.933 | 1.00 | 12.69 |
| 1191 | C | GLU | A | 369 | 45.492 | 9.450 | 93.577 | 1.00 | 11.12 |
| 1192 | O | GLU | A | 369 | 45.152 | 8.419 | 94.123 | 1.00 | 13.47 |
| 1193 | N | TYR | A | 370 | 46.760 | 9.692 | 93.255 | 1.00 | 8.69 |
| 1194 | CA | TYR | A | 370 | 47.778 | 8.683 | 93.538 | 1.00 | 9.43 |
| 1195 | CB | TYR | A | 370 | 49.137 | 9.069 | 92.949 | 1.00 | 13.30 |
| 1196 | CG | TYR | A | 370 | 49.432 | 8.500 | 91.586 | 1.00 | 16.34 |
| 1197 | CD1 | TYR | A | 370 | 49.209 | 9.234 | 90.429 | 1.00 | 13.30 |
| 1198 | CE1 | TYR | A | 370 | 49.548 | 8.742 | 89.210 | 1.00 | 6.00 |
| 1199 | CD2 | TYR | A | 370 | 49.985 | 7.247 | 91.463 | 1.00 | 21.23 |
| 1200 | CE2 | TYR | A | 370 | 50.313 | 6.725 | 90.234 | 1.00 | 18.57 |
| 1201 | CZ | TYR | A | 370 | 50.101 | 7.469 | 89.103 | 1.00 | 20.19 |
| 1202 | OH | TYR | A | 370 | 50.417 | 6.900 | 87.871 | 1.00 | 19.47 |
| 1203 | C | TYR | A | 370 | 47.926 | 8.547 | 95.038 | 1.00 | 11.30 |
| 1204 | O | TYR | A | 370 | 47.928 | 7.480 | 95.575 | 1.00 | 12.12 |
| 1205 | N | ALA | A | 371 | 48.027 | 9.658 | 95.734 | 1.00 | 16.86 |
| 1206 | CA | ALA | A | 371 | 48.202 | 9.549 | 97.163 | 1.00 | 18.13 |
| 1207 | CB | ALA | A | 371 | 48.167 | 10.925 | 97.793 | 1.00 | 16.28 |
| 1208 | C | ALA | A | 371 | 47.062 | 8.692 | 97.698 | 1.00 | 19.18 |
| 1209 | O | ALA | A | 371 | 47.258 | 7.734 | 98.469 | 1.00 | 17.78 |
| 1210 | N | LEU | A | 372 | 45.856 | 9.055 | 97.277 | 1.00 | 19.78 |
| 1211 | CA | LEU | A | 372 | 44.662 | 8.354 | 97.725 | 1.00 | 19.88 |
| 1212 | CB | LEU | A | 372 | 43.394 | 9.032 | 97.187 | 1.00 | 14.68 |
| 1213 | CG | LEU | A | 372 | 42.962 | 10.355 | 97.835 | 1.00 | 19.94 |
| 1214 | CD1 | LEU | A | 372 | 41.729 | 10.902 | 97.146 | 1.00 | 10.85 |
| 1215 | CD2 | LEU | A | 372 | 42.651 | 10.108 | 99.298 | 1.00 | 7.68 |
| 1216 | C | LEU | A | 372 | 44.656 | 6.868 | 97.332 | 1.00 | 22.11 |
| 1217 | O | LEU | A | 372 | 44.215 | 6.002 | 98.141 | 1.00 | 22.03 |
| 1218 | N | LEU | A | 373 | 45.175 | 6.552 | 96.133 | 1.00 | 15.88 |
| 1219 | CA | LEU | A | 373 | 45.137 | 5.166 | 95.695 | 1.00 | 10.66 |
| 1220 | CB | LEU | A | 373 | 45.372 | 5.030 | 94.196 | 1.00 | 8.65 |
| 1221 | CG | LEU | A | 373 | 44.655 | 3.840 | 93.598 | 1.00 | 6.32 |
| 1222 | CD1 | LEU | A | 373 | 43.285 | 4.260 | 93.208 | 1.00 | 5.25 |
| 1223 | CD2 | LEU | A | 373 | 45.422 | 3.376 | 92.400 | 1.00 | 20.15 |
| 1224 | C | LEU | A | 373 | 46.136 | 4.329 | 96.433 | 1.00 | 10.85 |
| 1225 | O | LEU | A | 373 | 45.889 | 3.129 | 96.632 | 1.00 | 14.08 |
| 1226 | N | ILE | A | 374 | 47.261 | 4.946 | 96.812 | 1.00 | 5.25 |
| 1227 | CA | ILE | A | 374 | 48.303 | 4.281 | 97.562 | 1.00 | 5.25 |
| 1228 | CB | ILE | A | 374 | 49.596 | 5.099 | 97.509 | 1.00 | 6.11 |
| 1229 | CG2 | ILE | A | 374 | 50.633 | 4.644 | 98.594 | 1.00 | 5.25 |
| 1230 | CG1 | ILE | A | 374 | 50.210 | 4.921 | 96.129 | 1.00 | 5.25 |
| 1231 | CD1 | ILE | A | 374 | 51.473 | 5.732 | 95.921 | 1.00 | 9.93 |
| 1232 | C | ILE | A | 374 | 47.800 | 4.089 | 99.012 | 1.00 | 8.80 |
| 1233 | O | ILE | A | 374 | 48.045 | 3.061 | 99.644 | 1.00 | 10.51 |
| 1234 | N | ALA | A | 375 | 47.051 | 5.045 | 99.538 | 1.00 | 11.95 |
| 1235 | CA | ALA | A | 375 | 46.522 | 4.857 | 100.879 | 1.00 | 11.55 |
| 1236 | CB | ALA | A | 375 | 45.772 | 6.112 | 101.348 | 1.00 | 18.37 |
| 1237 | C | ALA | A | 375 | 45.565 | 3.673 | 100.810 | 1.00 | 10.74 |
| 1238 | O | ALA | A | 375 | 45.736 | 2.692 | 101.512 | 1.00 | 12.44 |
| 1239 | N | ILE | A | 376 | 44.541 | 3.781 | 99.971 | 1.00 | 13.36 |
| 1240 | CA | ILE | A | 376 | 43.581 | 2.698 | 99.808 | 1.00 | 12.40 |
| 1241 | CB | ILE | A | 376 | 42.740 | 2.935 | 98.541 | 1.00 | 9.26 |
| 1242 | CG2 | ILE | A | 376 | 42.041 | 1.644 | 98.135 | 1.00 | 18.71 |
| 1243 | CG1 | ILE | A | 376 | 41.751 | 4.087 | 98.775 | 1.00 | 13.00 |
| 1244 | CD1 | ILE | A | 376 | 40.978 | 4.478 | 97.474 | 1.00 | 17.97 |
| 1245 | C | ILE | A | 376 | 44.306 | 1.338 | 99.702 | 1.00 | 12.50 |
| 1246 | O | ILE | A | 376 | 43.886 | 0.343 | 100.249 | 1.00 | 14.22 |
| 1247 | N | ASN | A | 377 | 45.431 | 1.305 | 99.014 | 1.00 | 15.95 |
| 1248 | CA | ASN | A | 377 | 46.217 | 0.075 | 98.837 | 1.00 | 12.41 |
| 1249 | CB | ASN | A | 377 | 47.332 | 0.356 | 97.817 | 1.00 | 15.42 |
| 1250 | CG | ASN | A | 377 | 48.167 | −0.873 | 97.487 | 1.00 | 13.49 |
| 1251 | OD1 | ASN | A | 377 | 49.214 | −1.104 | 98.085 | 1.00 | 20.76 |
| 1252 | ND2 | ASN | A | 377 | 47.721 | −1.650 | 96.522 | 1.00 | 5.51 |
| 1253 | C | ASN | A | 377 | 46.822 | −0.480 | 100.124 | 1.00 | 12.71 |
| 1254 | O | ASN | A | 377 | 46.791 | −1.665 | 100.344 | 1.00 | 12.86 |
| 1255 | N | ILE | A | 378 | 47.389 | 0.386 | 100.951 | 1.00 | 9.60 |
| 1256 | CA | ILE | A | 378 | 48.007 | −0.007 | 102.208 | 1.00 | 11.48 |
| 1257 | CB | ILE | A | 378 | 48.557 | 1.254 | 102.953 | 1.00 | 13.42 |
| 1258 | CG2 | ILE | A | 378 | 48.991 | 0.911 | 104.337 | 1.00 | 10.38 |
| 1259 | CG1 | ILE | A | 378 | 49.731 | 1.847 | 102.176 | 1.00 | 14.54 |
| 1260 | CD1 | ILE | A | 378 | 50.045 | 3.250 | 102.595 | 1.00 | 5.25 |
| 1261 | C | ILE | A | 378 | 47.018 | −0.709 | 103.143 | 1.00 | 11.55 |
| 1262 | O | ILE | A | 378 | 47.382 | −1.633 | 103.867 | 1.00 | 9.06 |
| 1263 | N | PHE | A | 379 | 45.775 | −0.244 | 103.141 | 1.00 | 10.61 |
| 1264 | CA | PHE | A | 379 | 44.724 | −0.812 | 103.989 | 1.00 | 12.29 |
| 1265 | CB | PHE | A | 379 | 43.781 | 0.293 | 104.436 | 1.00 | 7.47 |
| 1266 | CG | PHE | A | 379 | 44.440 | 1.315 | 105.268 | 1.00 | 11.02 |
| 1267 | CD1 | PHE | A | 379 | 44.988 | 0.966 | 106.506 | 1.00 | 9.62 |
| 1268 | CD2 | PHE | A | 379 | 44.434 | 2.646 | 104.866 | 1.00 | 11.32 |
| 1269 | CE1 | PHE | A | 379 | 45.520 | 1.966 | 107.363 | 1.00 | 15.85 |
| 1270 | CE2 | PHE | A | 379 | 44.951 | 3.653 | 105.686 | 1.00 | 18.67 |
| 1271 | CZ | PHE | A | 379 | 45.494 | 3.313 | 106.949 | 1.00 | 21.51 |
| 1272 | C | PHE | A | 379 | 43.914 | −1.918 | 103.334 | 1.00 | 10.55 |
| 1273 | O | PHE | A | 379 | 42.677 | −1.881 | 103.303 | 1.00 | 9.28 |
| 1274 | N | SER | A | 380 | 44.610 | −2.909 | 102.810 | 1.00 | 13.88 |
| 1275 | CA | SER | A | 380 | 43.917 | −4.015 | 102.177 | 1.00 | 17.32 |
| 1276 | CB | SER | A | 380 | 44.777 | −4.563 | 101.039 | 1.00 | 22.85 |
| 1277 | OG | SER | A | 380 | 44.923 | −3.634 | 99.988 | 1.00 | 11.41 |
| 1278 | C | SER | A | 380 | 43.699 | −5.094 | 103.259 | 1.00 | 17.60 |
| 1279 | O | SER | A | 380 | 44.653 | −5.748 | 103.690 | 1.00 | 17.72 |
| 1280 | N | ALA | A | 381 | 42.464 | −5.273 | 103.719 | 1.00 | 17.30 |
| 1281 | CA | ALA | A | 381 | 42.195 | −6.283 | 104.761 | 1.00 | 19.73 |
| 1282 | CB | ALA | A | 381 | 40.707 | −6.274 | 105.140 | 1.00 | 21.74 |
| 1283 | C | ALA | A | 381 | 42.613 | −7.724 | 104.431 | 1.00 | 18.37 |
| 1284 | O | ALA | A | 381 | 43.094 | −8.438 | 105.304 | 1.00 | 22.04 |
| 1285 | N | ASP | A | 382 | 42.438 | −8.141 | 103.178 | 1.00 | 14.77 |
| 1286 | CA | ASP | A | 382 | 42.754 | −9.486 | 102.772 | 1.00 | 13.45 |
| 1287 | CB | ASP | A | 382 | 42.035 | −9.807 | 101.457 | 1.00 | 13.39 |
| 1288 | CG | ASP | A | 382 | 42.630 | −9.082 | 100.246 | 1.00 | 30.72 |
| 1289 | OD1 | ASP | A | 382 | 43.378 | −8.081 | 100.397 | 1.00 | 22.68 |
| 1290 | OD2 | ASP | A | 382 | 42.334 | −9.519 | 99.114 | 1.00 | 32.64 |
| 1291 | C | ASP | A | 382 | 44.238 | −9.803 | 102.667 | 1.00 | 17.62 |
| 1292 | O | ASP | A | 382 | 44.605 | −10.924 | 102.314 | 1.00 | 20.22 |
| 1293 | N | ARG | A | 383 | 45.110 | −8.851 | 102.988 | 1.00 | 19.53 |
| 1294 | CA | ARG | A | 383 | 46.551 | −9.137 | 102.878 | 1.00 | 22.22 |
| 1295 | CB | ARG | A | 383 | 47.426 | −7.940 | 103.264 | 1.00 | 23.58 |
| 1296 | CG | ARG | A | 383 | 47.195 | −6.716 | 102.426 | 1.00 | 35.26 |
| 1297 | CD | ARG | A | 383 | 47.697 | −6.851 | 101.005 | 1.00 | 25.29 |
| 1298 | NE | ARG | A | 383 | 47.636 | −5.552 | 100.332 | 1.00 | 23.14 |
| 1299 | CZ | ARG | A | 383 | 48.123 | −5.319 | 99.126 | 1.00 | 23.43 |
| 1300 | NH1 | ARG | A | 383 | 48.710 | −6.289 | 98.461 | 1.00 | 27.91 |
| 1301 | NH2 | ARG | A | 383 | 48.006 | −4.121 | 98.580 | 1.00 | 27.85 |
| 1302 | C | ARG | A | 383 | 46.831 | −10.242 | 103.839 | 1.00 | 18.57 |
| 1303 | O | ARG | A | 383 | 46.226 | −10.305 | 104.920 | 1.00 | 25.15 |
| 1304 | N | PRO | A | 384 | 47.748 | −11.131 | 103.473 | 1.00 | 16.47 |
| 1305 | CD | PRO | A | 384 | 48.511 | −11.130 | 102.214 | 1.00 | 12.46 |
| 1306 | CA | PRO | A | 384 | 48.114 | −12.259 | 104.335 | 1.00 | 14.68 |
| 1307 | CB | PRO | A | 384 | 49.348 | −12.827 | 103.651 | 1.00 | 5.25 |
| 1308 | CG | PRO | A | 384 | 49.058 | −12.518 | 102.173 | 1.00 | 5.25 |
| 1309 | C | PRO | A | 384 | 48.422 | −11.868 | 105.799 | 1.00 | 19.79 |
| 1310 | O | PRO | A | 384 | 49.046 | −10.820 | 106.076 | 1.00 | 27.07 |
| 1311 | N | ASN | A | 385 | 47.966 | −12.706 | 106.729 | 1.00 | 20.14 |
| 1312 | CA | ASN | A | 385 | 48.279 | −12.565 | 108.159 | 1.00 | 20.58 |
| 1313 | CB | ASN | A | 385 | 49.832 | −12.711 | 108.399 | 1.00 | 20.36 |
| 1314 | CG | ASN | A | 385 | 50.474 | −13.822 | 107.532 | 1.00 | 31.32 |
| 1315 | OD1 | ASN | A | 385 | 50.776 | −13.633 | 106.344 | 1.00 | 41.94 |
| 1316 | ND2 | ASN | A | 385 | 50.647 | −14.986 | 108.121 | 1.00 | 25.03 |
| 1317 | C | ASN | A | 385 | 47.749 | −11.326 | 108.886 | 1.00 | 17.40 |
| 1318 | O | ASN | A | 385 | 48.181 | −10.969 | 109.977 | 1.00 | 12.75 |
| 1319 | N | VAL | A | 386 | 46.797 | −10.650 | 108.302 | 1.00 | 17.19 |
| 1320 | CA | VAL | A | 386 | 46.284 | −9.501 | 109.029 | 1.00 | 17.88 |
| 1321 | CB | VAL | A | 386 | 45.340 | −8.669 | 108.150 | 1.00 | 15.50 |
| 1322 | CG1 | VAL | A | 386 | 44.750 | −7.581 | 108.934 | 1.00 | 10.72 |
| 1323 | CG2 | VAL | A | 386 | 46.087 | −8.154 | 106.923 | 1.00 | 13.81 |
| 1324 | C | VAL | A | 386 | 45.513 | −10.006 | 110.268 | 1.00 | 21.92 |
| 1325 | O | VAL | A | 386 | 44.720 | −10.957 | 110.214 | 1.00 | 20.83 |
| 1326 | N | GLN | A | 387 | 45.761 | −9.372 | 111.400 | 1.00 | 26.53 |
| 1327 | CA | GLN | A | 387 | 45.084 | −9.753 | 112.637 | 1.00 | 30.70 |
| 1328 | CB | GLN | A | 387 | 45.891 | −9.282 | 113.836 | 1.00 | 37.12 |
| 1329 | CG | GLN | A | 387 | 46.969 | −10.223 | 114.318 | 1.00 | 44.09 |
| 1330 | CD | GLN | A | 387 | 47.415 | −9.894 | 115.738 | 1.00 | 47.02 |
| 1331 | OE1 | GLN | A | 387 | 48.257 | −10.592 | 116.289 | 1.00 | 48.50 |
| 1332 | NE2 | GLN | A | 387 | 46.846 | −8.825 | 116.338 | 1.00 | 39.63 |
| 1333 | C | GLN | A | 387 | 43.685 | −9.129 | 112.729 | 1.00 | 30.34 |
| 1334 | O | GLN | A | 387 | 42.701 | −9.777 | 113.055 | 1.00 | 31.77 |
| 1335 | N | GLU | A | 388 | 43.619 | −7.850 | 112.424 | 1.00 | 25.71 |
| 1336 | CA | GLU | A | 388 | 42.381 | −7.135 | 112.477 | 1.00 | 20.29 |
| 1337 | CB | GLU | A | 388 | 42.582 | −5.990 | 113.437 | 1.00 | 15.19 |
| 1338 | CG | GLU | A | 388 | 43.133 | −6.517 | 114.737 | 1.00 | 19.58 |
| 1339 | CD | GLU | A | 388 | 43.239 | −5.442 | 115.809 | 1.00 | 11.20 |
| 1340 | OE1 | GLU | A | 388 | 44.330 | −4.881 | 115.937 | 1.00 | 19.48 |
| 1341 | OE2 | GLU | A | 388 | 42.246 | −5.134 | 116.503 | 1.00 | 17.08 |
| 1342 | C | GLU | A | 388 | 41.930 | −6.653 | 111.094 | 1.00 | 16.35 |
| 1343 | O | GLU | A | 388 | 41.896 | −5.473 | 110.821 | 1.00 | 15.52 |
| 1344 | N | PRO | A | 389 | 41.589 | −7.586 | 110.202 | 1.00 | 16.54 |
| 1345 | CD | PRO | A | 389 | 41.398 | −9.024 | 110.443 | 1.00 | 16.80 |
| 1346 | CA | PRO | A | 389 | 41.146 | −7.232 | 108.857 | 1.00 | 20.31 |
| 1347 | CB | PRO | A | 389 | 40.773 | −8.579 | 108.250 | 1.00 | 24.57 |
| 1348 | CG | PRO | A | 389 | 40.322 | −9.336 | 109.442 | 1.00 | 16.51 |
| 1349 | C | PRO | A | 389 | 39.984 | −6.296 | 108.944 | 1.00 | 22.62 |
| 1350 | O | PRO | A | 389 | 40.076 | −5.171 | 108.451 | 1.00 | 29.81 |
| 1351 | N | GLY | A | 390 | 38.905 | −6.756 | 109.578 | 1.00 | 19.40 |
| 1352 | CA | GLY | A | 390 | 37.722 | −5.926 | 109.734 | 1.00 | 20.42 |
| 1353 | C | GLY | A | 390 | 38.076 | −4.507 | 110.166 | 1.00 | 21.49 |
| 1354 | O | GLY | A | 390 | 37.394 | −3.523 | 109.865 | 1.00 | 23.11 |
| 1355 | N | ARG | A | 391 | 39.173 | −4.408 | 110.898 | 1.00 | 22.62 |
| 1356 | CA | ARG | A | 391 | 39.645 | −3.130 | 111.360 | 1.00 | 19.89 |
| 1357 | CB | ARG | A | 391 | 40.538 | −3.343 | 112.583 | 1.00 | 14.12 |
| 1358 | CG | ARG | A | 391 | 40.581 | −2.157 | 113.490 | 1.00 | 18.23 |
| 1359 | CD | ARG | A | 391 | 41.360 | −2.447 | 114.786 | 1.00 | 13.23 |
| 1360 | NE | ARG | A | 391 | 42.102 | −1.258 | 115.228 | 1.00 | 22.11 |
| 1361 | CZ | ARG | A | 391 | 43.090 | −1.273 | 116.105 | 1.00 | 18.99 |
| 1362 | NH1 | ARG | A | 391 | 43.462 | −2.422 | 116.644 | 1.00 | 20.41 |
| 1363 | NH2 | ARG | A | 391 | 43.689 | −0.135 | 116.424 | 1.00 | 29.33 |
| 1364 | C | ARG | A | 391 | 40.407 | −2.458 | 110.187 | 1.00 | 18.96 |
| 1365 | O | ARG | A | 391 | 40.183 | −1.282 | 109.880 | 1.00 | 20.35 |
| 1366 | N | VAL | A | 392 | 41.298 | −3.199 | 109.529 | 1.00 | 15.96 |
| 1367 | CA | VAL | A | 392 | 42.020 | −2.656 | 108.400 | 1.00 | 17.16 |
| 1368 | CB | VAL | A | 392 | 42.878 | −3.720 | 107.771 | 1.00 | 13.89 |
| 1369 | CG1 | VAL | A | 392 | 43.494 | −3.210 | 106.464 | 1.00 | 8.04 |
| 1370 | CG2 | VAL | A | 392 | 43.951 | −4.098 | 108.761 | 1.00 | 9.26 |
| 1371 | C | VAL | A | 392 | 41.008 | −2.148 | 107.378 | 1.00 | 20.53 |
| 1372 | O | VAL | A | 392 | 41.144 | −1.061 | 106.829 | 1.00 | 22.78 |
| 1373 | N | GLU | A | 393 | 39.960 | −2.922 | 107.153 | 1.00 | 20.85 |
| 1374 | CA | GLU | A | 393 | 38.941 | −2.543 | 106.187 | 1.00 | 24.73 |
| 1375 | CB | GLU | A | 393 | 37.948 | −3.690 | 106.044 | 1.00 | 22.33 |
| 1376 | CG | GLU | A | 393 | 36.836 | −3.509 | 105.029 | 1.00 | 30.78 |
| 1377 | CD | GLU | A | 393 | 35.988 | −4.807 | 104.891 | 1.00 | 37.98 |
| 1378 | OE1 | GLU | A | 393 | 34.815 | −4.762 | 104.458 | 1.00 | 25.11 |
| 1379 | OE2 | GLU | A | 393 | 36.508 | −5.888 | 105.234 | 1.00 | 38.44 |
| 1380 | C | GLU | A | 393 | 38.241 | −1.255 | 106.594 | 1.00 | 26.29 |
| 1381 | O | GLU | A | 393 | 37.967 | −0.396 | 105.778 | 1.00 | 29.48 |
| 1382 | N | ALA | A | 394 | 37.967 | −1.109 | 107.873 | 1.00 | 25.71 |
| 1383 | CA | ALA | A | 394 | 37.295 | 0.083 | 108.330 | 1.00 | 23.23 |
| 1384 | CB | ALA | A | 394 | 37.029 | −0.004 | 109.876 | 1.00 | 5.25 |
| 1385 | C | ALA | A | 394 | 38.192 | 1.272 | 107.997 | 1.00 | 24.37 |
| 1386 | O | ALA | A | 394 | 37.699 | 2.322 | 107.552 | 1.00 | 20.89 |
| 1387 | N | LEU | A | 395 | 39.504 | 1.101 | 108.226 | 1.00 | 26.86 |
| 1388 | CA | LEU | A | 395 | 40.502 | 2.157 | 107.978 | 1.00 | 26.79 |
| 1389 | CB | LEU | A | 395 | 41.869 | 1.725 | 108.485 | 1.00 | 17.72 |
| 1390 | CG | LEU | A | 395 | 42.141 | 1.960 | 109.968 | 1.00 | 23.47 |
| 1391 | CD1 | LEU | A | 395 | 43.497 | 1.279 | 110.420 | 1.00 | 6.26 |
| 1392 | CD2 | LEU | A | 395 | 42.164 | 3.475 | 110.160 | 1.00 | 6.73 |
| 1393 | C | LEU | A | 395 | 40.631 | 2.547 | 106.513 | 1.00 | 31.66 |
| 1394 | O | LEU | A | 395 | 41.083 | 3.618 | 106.177 | 1.00 | 34.95 |
| 1395 | N | GLN | A | 396 | 40.185 | 1.688 | 105.624 | 1.00 | 33.84 |
| 1396 | CA | GLN | A | 396 | 40.314 | 1.993 | 104.228 | 1.00 | 29.48 |
| 1397 | CB | GLN | A | 396 | 40.298 | 0.680 | 103.433 | 1.00 | 31.37 |
| 1398 | CG | GLN | A | 396 | 40.659 | 0.831 | 101.968 | 1.00 | 30.28 |
| 1399 | CD | GLN | A | 396 | 40.428 | −0.434 | 101.167 | 1.00 | 27.29 |
| 1400 | OE1 | GLN | A | 396 | 39.296 | −0.842 | 100.941 | 1.00 | 33.68 |
| 1401 | NE2 | GLN | A | 396 | 41.501 | −1.066 | 100.748 | 1.00 | 31.43 |
| 1402 | C | GLN | A | 396 | 39.247 | 2.905 | 103.703 | 1.00 | 27.95 |
| 1403 | O | GLN | A | 396 | 39.502 | 3.852 | 102.986 | 1.00 | 34.21 |
| 1404 | N | GLN | A | 397 | 38.033 | 2.606 | 104.075 | 1.00 | 28.79 |
| 1405 | CA | GLN | A | 397 | 36.888 | 3.304 | 103.564 | 1.00 | 27.47 |
| 1406 | CB | GLN | A | 397 | 35.665 | 2.707 | 104.272 | 1.00 | 23.47 |
| 1407 | CG | GLN | A | 397 | 35.922 | 1.192 | 104.530 | 1.00 | 28.31 |
| 1408 | CD | GLN | A | 397 | 35.505 | 0.226 | 103.395 | 1.00 | 40.98 |
| 1409 | OE1 | GLN | A | 397 | 36.280 | −0.668 | 102.997 | 1.00 | 31.54 |
| 1410 | NE2 | GLN | A | 397 | 34.273 | 0.390 | 102.895 | 1.00 | 44.00 |
| 1411 | C | GLN | A | 397 | 36.913 | 4.804 | 103.523 | 1.00 | 24.58 |
| 1412 | O | GLN | A | 397 | 36.535 | 5.393 | 102.530 | 1.00 | 28.53 |
| 1413 | N | PRO | A | 398 | 37.409 | 5.444 | 104.559 | 1.00 | 23.95 |
| 1414 | CD | PRO | A | 398 | 37.715 | 4.894 | 105.881 | 1.00 | 26.73 |
| 1415 | CA | PRO | A | 398 | 37.437 | 6.914 | 104.520 | 1.00 | 23.31 |
| 1416 | CB | PRO | A | 398 | 38.221 | 7.287 | 105.775 | 1.00 | 19.47 |
| 1417 | CG | PRO | A | 398 | 37.979 | 6.161 | 106.703 | 1.00 | 37.68 |
| 1418 | C | PRO | A | 398 | 38.166 | 7.389 | 103.230 | 1.00 | 25.88 |
| 1419 | O | PRO | A | 398 | 37.798 | 8.397 | 102.623 | 1.00 | 25.59 |
| 1420 | N | TYR | A | 399 | 39.213 | 6.657 | 102.835 | 1.00 | 26.35 |
| 1421 | CA | TYR | A | 399 | 40.036 | 7.001 | 101.663 | 1.00 | 22.46 |
| 1422 | CB | TYR | A | 399 | 41.414 | 6.296 | 101.717 | 1.00 | 15.92 |
| 1423 | CG | TYR | A | 399 | 42.256 | 6.745 | 102.917 | 1.00 | 10.71 |
| 1424 | CD1 | TYR | A | 399 | 42.724 | 8.047 | 103.003 | 1.00 | 15.66 |
| 1425 | CE1 | TYR | A | 399 | 43.385 | 8.505 | 104.108 | 1.00 | 9.61 |
| 1426 | CD2 | TYR | A | 399 | 42.497 | 5.898 | 103.989 | 1.00 | 5.25 |
| 1427 | CE2 | TYR | A | 399 | 43.169 | 6.335 | 105.110 | 1.00 | 6.63 |
| 1428 | CZ | TYR | A | 399 | 43.615 | 7.654 | 105.169 | 1.00 | 17.65 |
| 1429 | OH | TYR | A | 399 | 44.307 | 8.110 | 106.287 | 1.00 | 18.05 |
| 1430 | C | TYR | A | 399 | 39.313 | 6.673 | 100.382 | 1.00 | 21.91 |
| 1431 | O | TYR | A | 399 | 39.317 | 7.488 | 99.450 | 1.00 | 17.12 |
| 1432 | N | VAL | A | 400 | 38.671 | 5.504 | 100.324 | 1.00 | 19.13 |
| 1433 | CA | VAL | A | 400 | 37.931 | 5.185 | 99.108 | 1.00 | 19.63 |
| 1434 | CB | VAL | A | 400 | 37.121 | 3.953 | 99.257 | 1.00 | 16.29 |
| 1435 | CG1 | VAL | A | 400 | 36.263 | 3.749 | 98.029 | 1.00 | 13.91 |
| 1436 | CG2 | VAL | A | 400 | 38.063 | 2.765 | 99.460 | 1.00 | 22.87 |
| 1437 | C | VAL | A | 400 | 37.003 | 6.358 | 98.859 | 1.00 | 25.95 |
| 1438 | O | VAL | A | 400 | 37.084 | 7.003 | 97.815 | 1.00 | 30.52 |
| 1439 | N | GLU | A | 401 | 36.147 | 6.663 | 99.836 | 1.00 | 28.51 |
| 1440 | CA | GLU | A | 401 | 35.241 | 7.812 | 99.723 | 1.00 | 28.24 |
| 1441 | CB | GLU | A | 401 | 34.688 | 8.215 | 101.081 | 1.00 | 14.53 |
| 1442 | CG | GLU | A | 401 | 33.522 | 7.390 | 101.523 | 1.00 | 45.77 |
| 1443 | CD | GLU | A | 401 | 32.410 | 8.230 | 102.120 | 1.00 | 42.44 |
| 1444 | OE1 | GLU | A | 401 | 31.240 | 8.032 | 101.702 | 1.00 | 39.71 |
| 1445 | OE2 | GLU | A | 401 | 32.721 | 9.073 | 102.992 | 1.00 | 43.94 |
| 1446 | C | GLU | A | 401 | 35.926 | 9.044 | 99.132 | 1.00 | 26.96 |
| 1447 | O | GLU | A | 401 | 35.481 | 9.581 | 98.112 | 1.00 | 29.78 |
| 1448 | N | ALA | A | 402 | 36.994 | 9.491 | 99.793 | 1.00 | 23.76 |
| 1449 | CA | ALA | A | 402 | 37.723 | 10.650 | 99.351 | 1.00 | 19.13 |
| 1450 | CB | ALA | A | 402 | 39.009 | 10.730 | 100.095 | 1.00 | 14.35 |
| 1451 | C | ALA | A | 402 | 37.959 | 10.539 | 97.841 | 1.00 | 22.89 |
| 1452 | O | ALA | A | 402 | 37.731 | 11.497 | 97.106 | 1.00 | 20.80 |
| 1453 | N | LEU | A | 403 | 38.369 | 9.360 | 97.365 | 1.00 | 23.41 |
| 1454 | CA | LEU | A | 403 | 38.604 | 9.184 | 95.934 | 1.00 | 20.90 |
| 1455 | CB | LEU | A | 403 | 39.263 | 7.845 | 95.665 | 1.00 | 15.78 |
| 1456 | CG | LEU | A | 403 | 39.819 | 7.745 | 94.244 | 1.00 | 7.31 |
| 1457 | CD1 | LEU | A | 403 | 40.970 | 8.686 | 94.102 | 1.00 | 10.15 |
| 1458 | CD2 | LEU | A | 403 | 40.244 | 6.321 | 93.975 | 1.00 | 9.87 |
| 1459 | C | LEU | A | 403 | 37.314 | 9.284 | 95.116 | 1.00 | 22.22 |
| 1460 | O | LEU | A | 403 | 37.296 | 9.931 | 94.062 | 1.00 | 20.72 |
| 1461 | N | LEU | A | 404 | 36.255 | 8.615 | 95.597 | 1.00 | 24.61 |
| 1462 | CA | LEU | A | 404 | 34.915 | 8.621 | 94.975 | 1.00 | 20.80 |
| 1463 | CB | LEU | A | 404 | 33.871 | 7.926 | 95.877 | 1.00 | 11.48 |
| 1464 | CG | LEU | A | 404 | 32.500 | 7.836 | 95.160 | 1.00 | 7.08 |
| 1465 | CD1 | LEU | A | 404 | 32.690 | 7.079 | 93.867 | 1.00 | 15.09 |
| 1466 | CD2 | LEU | A | 404 | 31.489 | 7.059 | 95.975 | 1.00 | 8.23 |
| 1467 | C | LEU | A | 404 | 34.444 | 10.062 | 94.760 | 1.00 | 22.50 |
| 1468 | O | LEU | A | 404 | 34.107 | 10.461 | 93.672 | 1.00 | 25.06 |
| 1469 | N | SER | A | 405 | 34.425 | 10.831 | 95.834 | 1.00 | 23.12 |
| 1470 | CA | SER | A | 405 | 34.015 | 12.209 | 95.793 | 1.00 | 20.64 |
| 1471 | CB | SER | A | 405 | 33.946 | 12.735 | 97.234 | 1.00 | 27.30 |
| 1472 | OG | SER | A | 405 | 33.432 | 14.056 | 97.321 | 1.00 | 45.53 |
| 1473 | C | SER | A | 405 | 34.963 | 13.058 | 94.950 | 1.00 | 20.08 |
| 1474 | O | SER | A | 405 | 34.521 | 13.960 | 94.283 | 1.00 | 2545 |
| 1475 | N | TYR | A | 406 | 36.258 | 12.768 | 94.982 | 1.00 | 18.80 |
| 1476 | CA | TYR | A | 406 | 37.234 | 13.539 | 94.232 | 1.00 | 14.69 |
| 1477 | CB | TYR | A | 406 | 38.635 | 13.072 | 94.548 | 1.00 | 9.34 |
| 1478 | CG | TYR | A | 406 | 39.703 | 13.963 | 93.990 | 1.00 | 14.57 |
| 1479 | CD1 | TYR | A | 406 | 39.971 | 15.181 | 94.559 | 1.00 | 21.04 |
| 1480 | CE1 | TYR | A | 406 | 41.052 | 15.975 | 94.109 | 1.00 | 20.89 |
| 1481 | CD2 | TYR | A | 406 | 40.510 | 13.544 | 92.944 | 1.00 | 18.62 |
| 1482 | CE2 | TYR | A | 406 | 41.574 | 14.315 | 92.490 | 1.00 | 15.48 |
| 1483 | CZ | TYR | A | 406 | 41.858 | 15.542 | 93.087 | 1.00 | 21.87 |
| 1484 | OH | TYR | A | 406 | 42.990 | 16.313 | 92.740 | 1.00 | 19.24 |
| 1485 | C | TYR | A | 406 | 36.984 | 13.351 | 92.760 | 1.00 | 16.56 |
| 1486 | O | TYR | A | 406 | 36.818 | 14.334 | 92.035 | 1.00 | 20.10 |
| 1487 | N | THR | A | 407 | 36.950 | 12.091 | 92.325 | 1.00 | 16.86 |
| 1488 | CA | THR | A | 407 | 36.709 | 11.765 | 90.920 | 1.00 | 16.53 |
| 1489 | CB | THR | A | 407 | 36.771 | 10.217 | 90.692 | 1.00 | 13.61 |
| 1490 | OG1 | THR | A | 407 | 35.825 | 9.557 | 91.545 | 1.00 | 6.85 |
| 1491 | CG2 | THR | A | 407 | 38.127 | 9.678 | 90.980 | 1.00 | 5.25 |
| 1492 | C | THR | A | 407 | 35.353 | 12.334 | 90.394 | 1.00 | 18.56 |
| 1493 | O | THR | A | 407 | 35.258 | 12.774 | 89.251 | 1.00 | 12.45 |
| 1494 | N | ARG | A | 408 | 34.319 | 12.323 | 91.234 | 1.00 | 22.92 |
| 1495 | CA | ARG | A | 408 | 33.022 | 12.882 | 90.872 | 1.00 | 27.72 |
| 1496 | CB | ARG | A | 408 | 32.055 | 12.823 | 92.050 | 1.00 | 35.75 |
| 1497 | CG | ARG | A | 408 | 31.042 | 11.676 | 92.011 | 1.00 | 46.40 |
| 1498 | CD | ARG | A | 408 | 30.033 | 11.897 | 93.119 | 1.00 | 67.06 |
| 1499 | NE | ARG | A | 408 | 29.113 | 10.778 | 93.340 | 1.00 | 78.74 |
| 1500 | CZ | ARG | A | 408 | 28.091 | 10.798 | 94.203 | 1.00 | 83.77 |
| 1501 | NH1 | ARG | A | 408 | 27.838 | 11.878 | 94.937 | 1.00 | 83.98 |
| 1502 | NH2 | ARG | A | 408 | 27.321 | 9.725 | 94.345 | 1.00 | 84.62 |
| 1503 | C | ARG | A | 408 | 33.190 | 14.357 | 90.464 | 1.00 | 29.46 |
| 1504 | O | ARG | A | 408 | 32.576 | 14.825 | 89.498 | 1.00 | 33.89 |
| 1505 | N | ILE | A | 409 | 34.009 | 15.088 | 91.213 | 1.00 | 25.53 |
| 1506 | CA | ILE | A | 409 | 34.298 | 16.482 | 90.918 | 1.00 | 23.48 |
| 1507 | CB | ILE | A | 409 | 35.024 | 17.123 | 92.140 | 1.00 | 24.09 |
| 1508 | CG2 | ILE | A | 409 | 35.445 | 18.538 | 91.872 | 1.00 | 21.70 |
| 1509 | CG1 | ILE | A | 409 | 34.096 | 17.139 | 93.334 | 1.00 | 20.16 |
| 1510 | CD1 | ILE | A | 409 | 34.872 | 17.171 | 94.668 | 1.00 | 11.98 |
| 1511 | C | ILE | A | 409 | 35.193 | 16.628 | 89.647 | 1.00 | 24.10 |
| 1512 | O | ILE | A | 409 | 34.942 | 17.466 | 88.767 | 1.00 | 24.66 |
| 1513 | N | LYS | A | 410 | 36.218 | 15.795 | 89.547 | 1.00 | 20.96 |
| 1514 | CA | LYS | A | 410 | 37.128 | 15.876 | 88.442 | 1.00 | 18.34 |
| 1515 | CB | LYS | A | 410 | 38.342 | 15.008 | 88.742 | 1.00 | 18.67 |
| 1516 | CG | LYS | A | 410 | 39.498 | 15.178 | 87.782 | 1.00 | 24.94 |
| 1517 | CD | LYS | A | 410 | 40.700 | 14.291 | 88.125 | 1.00 | 26.23 |
| 1518 | CE | LYS | A | 410 | 41.936 | 14.709 | 87.307 | 1.00 | 28.36 |
| 1519 | NZ | LYS | A | 410 | 43.165 | 13.963 | 87.652 | 1.00 | 16.11 |
| 1520 | C | LYS | A | 410 | 36.543 | 15.551 | 87.053 | 1.00 | 16.91 |
| 1521 | O | LYS | A | 410 | 36.891 | 16.178 | 86.066 | 1.00 | 21.62 |
| 1522 | N | ARG | A | 411 | 35.664 | 14.579 | 86.954 | 1.00 | 14.05 |
| 1523 | CA | ARG | A | 411 | 35.084 | 14.241 | 85.653 | 1.00 | 14.43 |
| 1524 | CB | ARG | A | 411 | 35.856 | 13.117 | 84.943 | 1.00 | 14.95 |
| 1525 | CG | ARG | A | 411 | 37.215 | 13.516 | 84.372 | 1.00 | 16.11 |
| 1526 | CD | ARG | A | 411 | 37.699 | 12.437 | 83.398 | 1.00 | 22.77 |
| 1527 | NE | ARG | A | 411 | 39.073 | 12.656 | 82.949 | 1.00 | 34.57 |
| 1528 | CZ | ARG | A | 411 | 39.712 | 11.852 | 82.102 | 1.00 | 36.23 |
| 1529 | NH1 | ARG | A | 411 | 39.082 | 10.786 | 81.615 | 1.00 | 31.20 |
| 1530 | NH2 | ARG | A | 411 | 40.980 | 12.099 | 81.768 | 1.00 | 21.00 |
| 1531 | C | ARG | A | 411 | 33.698 | 13.756 | 85.953 | 1.00 | 14.95 |
| 1532 | O | ARG | A | 411 | 33.415 | 12.577 | 85.853 | 1.00 | 17.23 |
| 1533 | N | PRO | A | 412 | 32.802 | 14.674 | 86.324 | 1.00 | 18.27 |
| 1534 | CD | PRO | A | 412 | 32.882 | 16.161 | 86.322 | 1.00 | 19.78 |
| 1535 | CA | PRO | A | 412 | 31.454 | 14.197 | 86.633 | 1.00 | 15.45 |
| 1536 | CB | PRO | A | 412 | 30.736 | 15.469 | 87.069 | 1.00 | 12.96 |
| 1537 | CG | PRO | A | 412 | 31.447 | 16.581 | 86.230 | 1.00 | 16.38 |
| 1538 | C | PRO | A | 412 | 30.799 | 13.533 | 85.453 | 1.00 | 16.13 |
| 1539 | O | PRO | A | 412 | 29.947 | 12.656 | 85.640 | 1.00 | 18.43 |
| 1540 | N | GLN | A | 413 | 31.181 | 13.958 | 84.246 | 1.00 | 17.14 |
| 1541 | CA | GLN | A | 413 | 30.588 | 13.387 | 83.063 | 1.00 | 21.06 |
| 1542 | CB | GLN | A | 413 | 30.729 | 14.366 | 81.922 | 1.00 | 28.30 |
| 1543 | CG | GLN | A | 413 | 29.656 | 15.455 | 81.871 | 1.00 | 51.45 |
| 1544 | CD | GLN | A | 413 | 29.866 | 16.617 | 82.862 | 1.00 | 64.03 |
| 1545 | OE1 | GLN | A | 413 | 30.935 | 17.243 | 82.917 | 1.00 | 65.04 |
| 1546 | NE2 | GLN | A | 413 | 28.826 | 16.917 | 83.635 | 1.00 | 70.87 |
| 1547 | C | GLN | A | 413 | 31.161 | 11.998 | 82.673 | 1.00 | 24.10 |
| 1548 | O | GLN | A | 413 | 30.859 | 11.473 | 81.583 | 1.00 | 26.59 |
| 1549 | N | ASP | A | 414 | 31.953 | 11.391 | 83.572 | 1.00 | 21.34 |
| 1550 | CA | ASP | A | 414 | 32.585 | 10.081 | 83.333 | 1.00 | 17.69 |
| 1551 | CB | ASP | A | 414 | 33.993 | 10.273 | 82.720 | 1.00 | 11.40 |
| 1552 | CG | ASP | A | 414 | 34.767 | 8.953 | 82.490 | 1.00 | 17.52 |
| 1553 | OD1 | ASP | A | 414 | 34.185 | 7.858 | 82.262 | 1.00 | 11.40 |
| 1554 | OD2 | ASP | A | 414 | 36.017 | 9.024 | 82.505 | 1.00 | 17.55 |
| 1555 | C | ASP | A | 414 | 32.641 | 9.236 | 84.618 | 1.00 | 19.02 |
| 1556 | O | ASP | A | 414 | 33.678 | 8.965 | 85.193 | 1.00 | 15.28 |
| 1557 | N | GLN | A | 415 | 31.473 | 8.788 | 85.028 | 1.00 | 22.23 |
| 1558 | CA | GLN | A | 415 | 31.332 | 7.973 | 86.199 | 1.00 | 24.58 |
| 1559 | CB | GLN | A | 415 | 29.868 | 7.618 | 86.405 | 1.00 | 33.91 |
| 1560 | CG | GLN | A | 415 | 29.010 | 8.757 | 86.882 | 1.00 | 47.58 |
| 1561 | CD | GLN | A | 415 | 27.579 | 8.328 | 87.105 | 1.00 | 62.81 |
| 1562 | OE1 | GLN | A | 415 | 26.779 | 9.069 | 87.695 | 1.00 | 61.90 |
| 1563 | NE2 | GLN | A | 415 | 27.238 | 7.117 | 86.626 | 1.00 | 66.91 |
| 1564 | C | GLN | A | 415 | 32.138 | 6.699 | 86.231 | 1.00 | 25.21 |
| 1565 | O | GLN | A | 415 | 32.548 | 6.289 | 87.313 | 1.00 | 26.32 |
| 1566 | N | LEU | A | 416 | 32.361 | 6.053 | 85.087 | 1.00 | 23.46 |
| 1567 | CA | LEU | A | 416 | 33.133 | 4.808 | 85.116 | 1.00 | 23.46 |
| 1568 | CB | LEU | A | 416 | 32.895 | 4.021 | 83.845 | 1.00 | 18.13 |
| 1569 | CG | LEU | A | 416 | 31.434 | 3.623 | 83.707 | 1.00 | 21.27 |
| 1570 | CD1 | LEU | A | 416 | 31.304 | 2.711 | 82.504 | 1.00 | 30.47 |
| 1571 | CD2 | LEU | A | 416 | 30.965 | 2.901 | 84.982 | 1.00 | 12.71 |
| 1572 | C | LEU | A | 416 | 34.643 | 4.968 | 85.336 | 1.00 | 26.85 |
| 1573 | O | LEU | A | 416 | 35.378 | 3.977 | 85.477 | 1.00 | 26.62 |
| 1574 | N | ARG | A | 417 | 35.112 | 6.214 | 85.371 | 1.00 | 28.16 |
| 1575 | CA | ARG | A | 417 | 36.537 | 6.484 | 85.608 | 1.00 | 26.52 |
| 1576 | CB | ARG | A | 417 | 36.852 | 7.963 | 85.360 | 1.00 | 26.71 |
| 1577 | CG | ARG | A | 417 | 38.309 | 8.248 | 85.436 | 1.00 | 12.42 |
| 1578 | CD | ARG | A | 417 | 38.606 | 9.705 | 85.462 | 1.00 | 15.47 |
| 1579 | NE | ARG | A | 417 | 40.045 | 9.914 | 85.624 | 1.00 | 19.89 |
| 1580 | CZ | ARG | A | 417 | 40.610 | 10.604 | 86.617 | 1.00 | 30.02 |
| 1581 | NH1 | ARG | A | 417 | 39.871 | 11.171 | 87.569 | 1.00 | 29.62 |
| 1582 | NH2 | ARG | A | 417 | 41.930 | 10.740 | 86.644 | 1.00 | 26.16 |
| 1583 | C | ARG | A | 417 | 36.982 | 6.087 | 87.043 | 1.00 | 26.85 |
| 1584 | O | ARG | A | 417 | 38.138 | 5.729 | 87.262 | 1.00 | 24.07 |
| 1585 | N | PHE | A | 418 | 36.082 | 6.172 | 88.020 | 1.00 | 23.22 |
| 1586 | CA | PHE | A | 418 | 36.464 | 5.738 | 89.349 | 1.00 | 25.25 |
| 1587 | CB | PHE | A | 418 | 35.414 | 6.142 | 90.366 | 1.00 | 20.37 |
| 1588 | CG | PHE | A | 418 | 35.672 | 5.594 | 91.731 | 1.00 | 26.53 |
| 1589 | CD1 | PHE | A | 418 | 36.893 | 5.814 | 92.362 | 1.00 | 28.94 |
| 1590 | CD2 | PHE | A | 418 | 34.721 | 4.821 | 92.373 | 1.00 | 23.82 |
| 1591 | CE1 | PHE | A | 418 | 37.165 | 5.259 | 93.611 | 1.00 | 18.88 |
| 1592 | CE2 | PHE | A | 418 | 34.992 | 4.268 | 93.611 | 1.00 | 30.12 |
| 1593 | CZ | PHE | A | 418 | 36.219 | 4.490 | 94.226 | 1.00 | 24.28 |
| 1594 | C | PHE | A | 418 | 36.678 | 4.195 | 89.385 | 1.00 | 24.71 |
| 1595 | O | PHE | A | 418 | 37.649 | 3.691 | 89.976 | 1.00 | 20.41 |
| 1596 | N | PRO | A | 419 | 35.781 | 3.428 | 88.748 | 1.00 | 24.57 |
| 1597 | CD | PRO | A | 419 | 34.436 | 3.778 | 88.277 | 1.00 | 17.11 |
| 1598 | CA | PRO | A | 419 | 35.958 | 1.971 | 88.763 | 1.00 | 30.07 |
| 1599 | CB | PRO | A | 419 | 34.645 | 1.457 | 88.163 | 1.00 | 34.07 |
| 1600 | CG | PRO | A | 419 | 33.672 | 2.513 | 88.561 | 1.00 | 28.67 |
| 1601 | C | PRO | A | 419 | 37.183 | 1.537 | 87.946 | 1.00 | 31.08 |
| 1602 | O | PRO | A | 419 | 37.791 | 0.494 | 88.214 | 1.00 | 28.61 |
| 1603 | N | ARG | A | 420 | 37.535 | 2.326 | 86.931 | 1.00 | 32.83 |
| 1604 | CA | ARG | A | 420 | 38.698 | 1.973 | 86.116 | 1.00 | 29.40 |
| 1605 | CB | ARG | A | 420 | 38.885 | 2.953 | 84.965 | 1.00 | 26.01 |
| 1606 | CG | ARG | A | 420 | 38.051 | 2.688 | 83.774 | 1.00 | 15.64 |
| 1607 | CD | ARG | A | 420 | 38.357 | 3.783 | 82.682 | 1.00 | 16.77 |
| 1608 | NE | ARG | A | 420 | 37.213 | 3.819 | 81.800 | 1.00 | 32.31 |
| 1609 | CZ | ARG | A | 420 | 36.333 | 4.810 | 81.720 | 1.00 | 32.34 |
| 1610 | NH1 | ARG | A | 420 | 36.464 | 5.912 | 82.454 | 1.00 | 22.18 |
| 1611 | NH2 | ARG | A | 420 | 35.271 | 4.640 | 80.946 | 1.00 | 42.24 |
| 1612 | C | ARG | A | 420 | 39.909 | 2.045 | 87.046 | 1.00 | 26.85 |
| 1613 | O | ARG | A | 420 | 40.874 | 1.278 | 86.917 | 1.00 | 28.56 |
| 1614 | N | MET | A | 421 | 39.830 | 2.972 | 87.996 | 1.00 | 23.40 |
| 1615 | CA | MET | A | 421 | 40.909 | 3.172 | 88.942 | 1.00 | 22.94 |
| 1616 | CB | MET | A | 421 | 40.694 | 4.477 | 89.741 | 1.00 | 15.02 |
| 1617 | CG | MET | A | 421 | 41.658 | 5.548 | 89.351 | 1.00 | 15.86 |
| 1618 | SD | MET | A | 421 | 41.304 | 7.133 | 90.071 | 1.00 | 19.36 |
| 1619 | CE | MET | A | 421 | 40.500 | 8.004 | 88.644 | 1.00 | 23.77 |
| 1620 | C | MET | A | 421 | 41.048 | 1.971 | 89.872 | 1.00 | 20.18 |
| 1621 | O | MET | A | 421 | 42.110 | 1.333 | 89.896 | 1.00 | 18.21 |
| 1622 | N | LEU | A | 422 | 39.990 | 1.680 | 90.630 | 1.00 | 18.63 |
| 1623 | CA | LEU | A | 422 | 40.013 | 0.549 | 91.531 | 1.00 | 20.59 |
| 1624 | CB | LEU | A | 422 | 38.633 | 0.250 | 92.119 | 1.00 | 19.88 |
| 1625 | CG | LEU | A | 422 | 38.057 | 1.393 | 92.970 | 1.00 | 20.81 |
| 1626 | CD1 | LEU | A | 422 | 36.812 | 0.964 | 93.694 | 1.00 | 26.59 |
| 1627 | CD2 | LEU | A | 422 | 39.073 | 1.825 | 93.941 | 1.00 | 27.12 |
| 1628 | C | LEU | A | 422 | 40.513 | −0.635 | 90.736 | 1.00 | 22.93 |
| 1629 | O | LEU | A | 422 | 41.365 | −1.384 | 91.219 | 1.00 | 27.81 |
| 1630 | N | MET | A | 423 | 40.022 | −0.794 | 89.511 | 1.00 | 21.99 |
| 1631 | CA | MET | A | 423 | 40.489 | −1.888 | 88.652 | 1.00 | 23.39 |
| 1632 | CB | MET | A | 423 | 40.015 | −1.632 | 87.207 | 1.00 | 23.04 |
| 1633 | CG | MET | A | 423 | 39.307 | −2.819 | 86.610 | 1.00 | 34.27 |
| 1634 | SD | MET | A | 423 | 37.975 | −3.396 | 87.683 | 1.00 | 34.89 |
| 1635 | CE | MET | A | 423 | 36.767 | −2.077 | 87.334 | 1.00 | 35.94 |
| 1636 | C | MET | A | 423 | 42.046 | −2.093 | 88.672 | 1.00 | 20.11 |
| 1637 | O | MET | A | 423 | 42.528 | −3.222 | 88.577 | 1.00 | 17.25 |
| 1638 | N | LYS | A | 424 | 42.817 | −1.009 | 88.795 | 1.00 | 17.68 |
| 1639 | CA | LYS | A | 424 | 44.271 | −1.103 | 88.827 | 1.00 | 17.31 |
| 1640 | CB | LYS | A | 424 | 44.884 | 0.294 | 88.816 | 1.00 | 16.49 |
| 1641 | CG | LYS | A | 424 | 44.606 | 0.998 | 87.538 | 1.00 | 22.55 |
| 1642 | CD | LYS | A | 424 | 45.189 | 0.203 | 86.352 | 1.00 | 31.18 |
| 1643 | CE | LYS | A | 424 | 44.870 | 0.841 | 85.021 | 1.00 | 34.69 |
| 1644 | NZ | LYS | A | 424 | 43.422 | 0.627 | 84.683 | 1.00 | 47.10 |
| 1645 | C | LYS | A | 424 | 44.765 | −1.897 | 90.026 | 1.00 | 16.52 |
| 1646 | O | LYS | A | 424 | 45.869 | −2.485 | 89.989 | 1.00 | 13.57 |
| 1647 | N | LEU | A | 425 | 43.965 | −1.891 | 91.095 | 1.00 | 14.69 |
| 1648 | CA | LEU | A | 425 | 44.294 | −2.673 | 92.271 | 1.00 | 13.33 |
| 1649 | CB | LEU | A | 425 | 43.290 | −2.400 | 93.397 | 1.00 | 10.78 |
| 1650 | CG | LEU | A | 425 | 43.319 | −0.981 | 93.932 | 1.00 | 14.31 |
| 1651 | CD1 | LEU | A | 425 | 42.085 | −0.776 | 94.736 | 1.00 | 22.37 |
| 1652 | CD2 | LEU | A | 425 | 44.560 | −0.743 | 94.781 | 1.00 | 5.26 |
| 1653 | C | LEU | A | 425 | 44.251 | −4.162 | 91.878 | 1.00 | 10.81 |
| 1654 | O | LEU | A | 425 | 44.988 | −4.989 | 92.428 | 1.00 | 14.13 |
| 1655 | N | VAL | A | 426 | 43.378 | −4.501 | 90.932 | 1.00 | 10.71 |
| 1656 | CA | VAL | A | 426 | 43.249 | −5.878 | 90.490 | 1.00 | 13.91 |
| 1657 | CB | VAL | A | 426 | 42.128 | −6.050 | 89.453 | 1.00 | 14.20 |
| 1658 | CG1 | VAL | A | 426 | 42.100 | −7.491 | 89.014 | 1.00 | 9.66 |
| 1659 | CG2 | VAL | A | 426 | 40.753 | −5.620 | 90.047 | 1.00 | 13.13 |
| 1660 | C | VAL | A | 426 | 44.569 | −6.298 | 89.853 | 1.00 | 16.67 |
| 1661 | O | VAL | A | 426 | 45.161 | −7.333 | 90.177 | 1.00 | 20.54 |
| 1662 | N | SER | A | 427 | 45.053 | −5.463 | 88.957 | 1.00 | 13.21 |
| 1663 | CA | SER | A | 427 | 46.295 | −5.754 | 88.293 | 1.00 | 14.68 |
| 1664 | CB | SER | A | 427 | 46.423 | −4.782 | 87.146 | 1.00 | 20.09 |
| 1665 | CG | SER | A | 427 | 45.203 | −4.792 | 86.410 | 1.00 | 20.17 |
| 1666 | C | SER | A | 427 | 47.543 | −5.739 | 89.191 | 1.00 | 18.31 |
| 1667 | O | SER | A | 427 | 48.510 | −6.495 | 88.914 | 1.00 | 18.50 |
| 1668 | N | LEU | A | 428 | 47.532 | −4.897 | 90.239 | 1.00 | 16.56 |
| 1669 | CA | LEU | A | 428 | 48.670 | −4.845 | 91.150 | 1.00 | 14.64 |
| 1670 | CB | LEU | A | 428 | 48.538 | −3.709 | 92.179 | 1.00 | 9.37 |
| 1671 | CG | LEU | A | 428 | 48.587 | −2.257 | 91.679 | 1.00 | 17.69 |
| 1672 | CD1 | LEU | A | 428 | 48.208 | −1.301 | 92.778 | 1.00 | 18.66 |
| 1673 | CD2 | LEU | A | 428 | 49.967 | −1.929 | 91.201 | 1.00 | 12.21 |
| 1674 | C | LEU | A | 428 | 48.832 | −6.193 | 91.869 | 1.00 | 13.83 |
| 1675 | O | LEU | A | 428 | 49.929 | −6.532 | 92.339 | 1.00 | 11.00 |
| 1676 | N | ARG | A | 429 | 47.758 | −6.978 | 91.958 | 1.00 | 11.73 |
| 1677 | CA | ARG | A | 429 | 47.920 | −8.256 | 92.630 | 1.00 | 17.81 |
| 1678 | CB | ARG | A | 429 | 46.590 | −8.897 | 93.001 | 1.00 | 22.40 |
| 1679 | CG | ARG | A | 429 | 45.912 | −8.348 | 94.206 | 1.00 | 9.68 |
| 1680 | CD | ARG | A | 429 | 46.815 | −8.382 | 95.420 | 1.00 | 9.57 |
| 1681 | NE | ARG | A | 429 | 46.186 | −7.536 | 96.441 | 1.00 | 13.29 |
| 1682 | CZ | ARG | A | 429 | 45.247 | −7.954 | 97.279 | 1.00 | 8.67 |
| 1683 | NH1 | ARG | A | 429 | 44.855 | −9.209 | 97.261 | 1.00 | 16.20 |
| 1684 | NH2 | ARG | A | 429 | 44.618 | −7.101 | 98.053 | 1.00 | 16.87 |
| 1685 | C | ARG | A | 429 | 48.648 | −9.168 | 91.678 | 1.00 | 22.72 |
| 1686 | O | ARG | A | 429 | 49.624 | −9.814 | 92.064 | 1.00 | 27.52 |
| 1687 | N | THR | A | 430 | 48.178 | −9.237 | 90.430 | 1.00 | 24.76 |
| 1688 | CA | THR | A | 430 | 48.843 | −10.085 | 89.422 | 1.00 | 22.85 |
| 1689 | CB | THR | A | 430 | 48.187 | −9.960 | 88.024 | 1.00 | 17.87 |
| 1690 | OG1 | THR | A | 430 | 46.864 | −10.525 | 88.069 | 1.00 | 15.01 |
| 1691 | CG2 | THR | A | 430 | 49.011 | −10.747 | 86.988 | 1.00 | 13.65 |
| 1692 | C | THR | A | 430 | 50.339 | −9.712 | 89.333 | 1.00 | 22.44 |
| 1693 | O | THR | A | 430 | 51.227 | −10.591 | 89.324 | 1.00 | 18.36 |
| 1694 | N | LEU | A | 431 | 50.610 | −8.407 | 89.302 | 1.00 | 20.10 |
| 1695 | CA | LEU | A | 431 | 51.986 | −7.944 | 89.248 | 1.00 | 19.15 |
| 1696 | CB | LEU | A | 431 | 52.032 | −6.411 | 89.165 | 1.00 | 20.90 |
| 1697 | CG | LEU | A | 431 | 51.960 | −5.865 | 87.725 | 1.00 | 18.63 |
| 1698 | CD1 | LEU | A | 431 | 50.846 | −4.891 | 87.630 | 1.00 | 17.52 |
| 1699 | CD2 | LEU | A | 431 | 53.248 | −5.193 | 87.356 | 1.00 | 23.80 |
| 1700 | C | LEU | A | 431 | 52.762 | −8.448 | 90.457 | 1.00 | 17.38 |
| 1701 | O | LEU | A | 431 | 53.877 | −8.911 | 90.333 | 1.00 | 20.17 |
| 1702 | N | SER | A | 432 | 52.148 | −8.372 | 91.629 | 1.00 | 14.68 |
| 1703 | CA | SER | A | 432 | 52.763 | −8.830 | 92.861 | 1.00 | 9.40 |
| 1704 | CB | SER | A | 432 | 51.801 | −8.597 | 94.006 | 1.00 | 13.48 |
| 1705 | OG | SER | A | 432 | 52.423 | −8.642 | 95.267 | 1.00 | 13.96 |
| 1706 | C | SER | A | 432 | 53.084 | −10.315 | 92.737 | 1.00 | 13.39 |
| 1707 | O | SER | A | 432 | 54.115 | −10.763 | 93.251 | 1.00 | 15.02 |
| 1708 | N | SER | A | 433 | 52.235 | −11.086 | 92.042 | 1.00 | 13.33 |
| 1709 | CA | SER | A | 433 | 52.542 | −12.518 | 91.866 | 1.00 | 12.64 |
| 1710 | CB | SER | A | 433 | 51.368 | −13.308 | 91.295 | 1.00 | 12.54 |
| 1711 | OG | SER | A | 433 | 50.372 | −13.577 | 92.249 | 1.00 | 27.96 |
| 1712 | C | SER | A | 433 | 53.710 | −12.707 | 90.906 | 1.00 | 16.54 |
| 1713 | O | SER | A | 433 | 54.607 | −13.521 | 91.180 | 1.00 | 19.09 |
| 1714 | N | VAL | A | 434 | 53.694 | −11.961 | 89.790 | 1.00 | 13.12 |
| 1715 | CA | VAL | A | 434 | 54.728 | −12.068 | 88.790 | 1.00 | 11.80 |
| 1716 | CB | VAL | A | 434 | 54.434 | −11.119 | 87.641 | 1.00 | 21.88 |
| 1717 | CG1 | VAL | A | 434 | 55.491 | −11.241 | 86.523 | 1.00 | 34.34 |
| 1718 | CG2 | VAL | A | 434 | 53.098 | −11.466 | 87.095 | 1.00 | 23.02 |
| 1719 | C | VAL | A | 434 | 56.083 | −11.765 | 89.401 | 1.00 | 12.40 |
| 1720 | O | VAL | A | 434 | 57.082 | −12.486 | 89.125 | 1.00 | 5.25 |
| 1721 | N | HIS | A | 435 | 56.091 | −10.710 | 90.235 | 1.00 | 13.26 |
| 1722 | CA | HIS | A | 435 | 57.270 | −10.239 | 90.960 | 1.00 | 10.05 |
| 1723 | CB | HIS | A | 435 | 56.916 | −9.025 | 91.765 | 1.00 | 10.18 |
| 1724 | CG | HIS | A | 435 | 57.873 | −8.751 | 92.881 | 1.00 | 19.96 |
| 1725 | CD2 | HIS | A | 435 | 57.754 | −8.925 | 94.224 | 1.00 | 18.04 |
| 1726 | ND1 | HIS | A | 435 | 59.117 | −8.196 | 92.675 | 1.00 | 24.89 |
| 1727 | CE1 | HIS | A | 435 | 59.718 | −8.032 | 93.842 | 1.00 | 27.76 |
| 1728 | NE2 | HIS | A | 435 | 58.912 | −8.468 | 94.796 | 1.00 | 18.59 |
| 1729 | C | HIS | A | 435 | 57.785 | −11.301 | 91.901 | 1.00 | 13.96 |
| 1730 | O | HIS | A | 435 | 58.962 | −11.421 | 92.154 | 1.00 | 12.84 |
| 1731 | N | SER | A | 436 | 56.880 | −12.090 | 92.439 | 1.00 | 22.00 |
| 1732 | CA | SER | A | 436 | 57.312 | −13.147 | 93.328 | 1.00 | 25.75 |
| 1733 | CB | SER | A | 436 | 56.101 | −13.745 | 94.059 | 1.00 | 18.10 |
| 1734 | OG | SER | A | 436 | 56.542 | −14.368 | 95.254 | 1.00 | 39.95 |
| 1735 | C | SER | A | 436 | 58.043 | −14.205 | 92.482 | 1.00 | 26.71 |
| 1736 | O | SER | A | 436 | 59.047 | −14.772 | 92.925 | 1.00 | 29.44 |
| 1737 | N | GLU | A | 437 | 57.538 | −14.459 | 91.271 | 1.00 | 26.62 |
| 1738 | CA | GLU | A | 437 | 58.157 | −15.438 | 90.392 | 1.00 | 27.61 |
| 1739 | CB | GLU | A | 437 | 57.231 | −15.781 | 89.211 | 1.00 | 32.32 |
| 1740 | CG | GLU | A | 437 | 55.970 | −16.545 | 89.640 | 1.00 | 43.49 |
| 1741 | CD | GLU | A | 437 | 54.992 | −16.826 | 88.517 | 1.00 | 44.02 |
| 1742 | OE1 | GLU | A | 437 | 54.224 | −15.916 | 88.110 | 1.00 | 39.81 |
| 1743 | OE2 | GLU | A | 437 | 55.002 | −17.978 | 88.033 | 1.00 | 52.54 |
| 1744 | C | GLU | A | 437 | 59.476 | −14.858 | 89.907 | 1.00 | 30.11 |
| 1745 | O | GLU | A | 437 | 60.347 | −15.573 | 89.446 | 1.00 | 32.93 |
| 1746 | N | GLN | A | 438 | 59.623 | −13.545 | 90.038 | 1.00 | 29.79 |
| 1747 | CA | GLN | A | 438 | 60.845 | −12.863 | 89.658 | 1.00 | 23.83 |
| 1748 | CB | GLN | A | 438 | 60.570 | −11.387 | 89.459 | 1.00 | 22.03 |
| 1749 | CG | GLN | A | 438 | 61.790 | −10.526 | 89.384 | 1.00 | 24.80 |
| 1750 | CD | GLN | A | 438 | 62.679 | −10.921 | 88.263 | 1.00 | 23.62 |
| 1751 | OE1 | GLN | A | 438 | 62.559 | −10.414 | 87.131 | 1.00 | 17.75 |
| 1752 | NE2 | GLN | A | 438 | 63.570 | −11.867 | 88.549 | 1.00 | 10.79 |
| 1753 | C | GLN | A | 438 | 61.845 | −13.042 | 90.788 | 1.00 | 24.89 |
| 1754 | O | GLN | A | 438 | 63.035 | −13.107 | 90.544 | 1.00 | 25.50 |
| 1755 | N | VAL | A | 439 | 61.347 | −13.111 | 92.025 | 1.00 | 29.45 |
| 1756 | CA | VAL | A | 439 | 62.176 | −13.291 | 93.222 | 1.00 | 30.36 |
| 1757 | CB | VAL | A | 439 | 61.360 | −13.040 | 94.525 | 1.00 | 31.12 |
| 1758 | CG1 | VAL | A | 439 | 62.202 | −13.343 | 95.744 | 1.00 | 35.24 |
| 1759 | CG2 | VAL | A | 439 | 60.902 | −11.601 | 94.585 | 1.00 | 28.21 |
| 1760 | C | VAL | A | 439 | 62.650 | −14.736 | 93.238 | 1.00 | 32.10 |
| 1761 | O | VAL | A | 439 | 63.835 | −15.036 | 93.419 | 1.00 | 34.80 |
| 1762 | N | PHE | A | 440 | 61.680 | −15.618 | 93.062 | 1.00 | 31.65 |
| 1763 | CA | PHE | A | 440 | 61.884 | −17.048 | 93.006 | 1.00 | 33.36 |
| 1764 | CB | PHE | A | 440 | 60.559 | −17.695 | 92.602 | 1.00 | 43.52 |
| 1765 | CG | PHE | A | 440 | 60.697 | −19.095 | 92.091 | 1.00 | 52.34 |
| 1766 | CD1 | PHE | A | 440 | 61.337 | −19.355 | 90.866 | 1.00 | 53.44 |
| 1767 | CD2 | PHE | A | 440 | 60.253 | −20.157 | 92.866 | 1.00 | 51.37 |
| 1768 | CE1 | PHE | A | 440 | 61.542 | −20.657 | 90.437 | 1.00 | 58.67 |
| 1769 | CE2 | PHE | A | 440 | 60.453 | −21.463 | 92.450 | 1.00 | 56.99 |
| 1770 | CZ | PHE | A | 440 | 61.102 | −21.721 | 91.234 | 1.00 | 59.17 |
| 1771 | C | PHE | A | 440 | 62.957 | −17.413 | 91.992 | 1.00 | 33.01 |
| 1772 | O | PHE | A | 440 | 63.773 | −18.282 | 92.232 | 1.00 | 32.92 |
| 1773 | N | ALA | A | 441 | 62.910 | −16.770 | 90.837 | 1.00 | 30.92 |
| 1774 | CA | ALA | A | 441 | 63.867 | −17.041 | 89.795 | 1.00 | 33.32 |
| 1775 | CB | ALA | A | 441 | 63.280 | −16.615 | 88.443 | 1.00 | 28.33 |
| 1776 | C | ALA | A | 441 | 65.171 | −16.312 | 90.056 | 1.00 | 34.78 |
| 1777 | O | ALA | A | 441 | 66.236 | −16.681 | 89.558 | 1.00 | 34.01 |
| 1778 | N | LEU | A | 442 | 65.078 | −15.250 | 90.838 | 1.00 | 40.36 |
| 1779 | CA | LEU | A | 442 | 66.254 | −14.451 | 91.150 | 1.00 | 40.69 |
| 1780 | CB | LEU | A | 442 | 65.871 | −13.231 | 91.979 | 1.00 | 38.10 |
| 1781 | CG | LEU | A | 442 | 65.739 | −11.948 | 91.209 | 1.00 | 35.38 |
| 1782 | CD1 | LEU | A | 442 | 65.584 | −10.865 | 92.176 | 1.00 | 28.28 |
| 1783 | CD2 | LEU | A | 442 | 66.983 | −11.755 | 90.407 | 1.00 | 39.43 |
| 1784 | C | LEU | A | 442 | 67.244 | −15.279 | 91.931 | 1.00 | 43.23 |
| 1785 | O | LEU | A | 442 | 68.394 | −15.408 | 91.548 | 1.00 | 43.93 |
| 1786 | N | ARG | A | 443 | 66.784 | −15.849 | 93.033 | 1.00 | 45.47 |
| 1787 | CA | ARG | A | 443 | 67.651 | −16.635 | 93.885 | 1.00 | 51.75 |
| 1788 | CB | ARG | A | 443 | 66.853 | −17.149 | 95.070 | 1.00 | 54.53 |
| 1789 | CG | ARG | A | 443 | 65.760 | −18.115 | 94.680 | 1.00 | 56.75 |
| 1790 | CD | ARG | A | 443 | 65.017 | −18.637 | 95.882 | 1.00 | 69.71 |
| 1791 | NE | ARG | A | 443 | 65.428 | −18.001 | 97.135 | 1.00 | 81.95 |
| 1792 | CZ | ARG | A | 443 | 65.400 | −18.623 | 98.302 | 1.00 | 78.74 |
| 1793 | NH1 | ARG | A | 443 | 64.987 | −19.875 | 98.346 | 1.00 | 79.02 |
| 1794 | NH2 | ARG | A | 443 | 65.783 | −17.997 | 99.406 | 1.00 | 83.70 |
| 1795 | C | ARG | A | 443 | 68.255 | −17.801 | 93.096 | 1.00 | 54.47 |
| 1796 | O | ARG | A | 443 | 69.404 | −18.203 | 93.327 | 1.00 | 56.52 |
| 1797 | N | LEU | A | 444 | 67.474 | −18.336 | 92.157 | 1.00 | 57.46 |
| 1798 | CA | LEU | A | 444 | 67.931 | −19.431 | 91.299 | 1.00 | 56.62 |
| 1799 | CB | LEU | A | 444 | 66.830 | −19.831 | 90.312 | 1.00 | 59.25 |
| 1800 | CG | LEU | A | 444 | 65.995 | −21.008 | 90.831 | 1.00 | 58.14 |
| 1801 | CD1 | LEU | A | 444 | 64.687 | −21.069 | 90.096 | 1.00 | 61.54 |
| 1802 | CD2 | LEU | A | 444 | 66.769 | −22.284 | 90.629 | 1.00 | 63.49 |
| 1803 | C | LEU | A | 444 | 69.208 | −19.055 | 90.579 | 1.00 | 54.88 |
| 1804 | O | LEU | A | 444 | 69.894 | −19.897 | 90.078 | 1.00 | 53.96 |
| 1805 | N | GLN | A | 445 | 69.519 | −17.770 | 90.536 | 1.00 | 60.24 |
| 1806 | CA | GLN | A | 445 | 70.760 | −17.294 | 89.939 | 1.00 | 64.02 |
| 1807 | CB | GLN | A | 445 | 70.555 | −15.973 | 89.182 | 1.00 | 62.89 |
| 1808 | CG | GLN | A | 445 | 70.029 | −16.186 | 87.791 | 1.00 | 70.48 |
| 1809 | CD | GLN | A | 445 | 69.743 | −14.899 | 87.019 | 1.00 | 79.70 |
| 1810 | OE1 | GLN | A | 445 | 69.949 | −13.787 | 87.513 | 1.00 | 83.56 |
| 1811 | NE2 | GLN | A | 445 | 69.266 | −15.055 | 85.790 | 1.00 | 75.40 |
| 1812 | C | GLN | A | 445 | 71.682 | −17.052 | 91.137 | 1.00 | 66.51 |
| 1813 | O | GLN | A | 445 | 71.462 | −17.588 | 92.244 | 1.00 | 61.81 |
| 1814 | N | ASP | A | 446 | 72.730 | −16.262 | 90.925 | 1.00 | 70.85 |
| 1815 | CA | ASP | A | 446 | 73.628 | −15.959 | 92.035 | 1.00 | 72.12 |
| 1816 | CB | ASP | A | 446 | 75.105 | −15.935 | 91.595 | 1.00 | 76.38 |
| 1817 | CG | ASP | A | 446 | 76.046 | −15.907 | 92.793 | 1.00 | 83.33 |
| 1818 | OD1 | ASP | A | 446 | 76.229 | −16.979 | 93.418 | 1.00 | 82.21 |
| 1819 | OD2 | ASP | A | 446 | 76.572 | −14.813 | 93.137 | 1.00 | 83.93 |
| 1820 | C | ASP | A | 446 | 73.217 | −14.568 | 92.562 | 1.00 | 68.79 |
| 1821 | O | ASP | A | 446 | 73.930 | −13.925 | 93.345 | 1.00 | 72.15 |
| 1822 | N | LYS | A | 447 | 72.054 | −14.110 | 92.123 | 1.00 | 61.71 |
| 1823 | CA | LYS | A | 447 | 71.568 | −12.820 | 92.537 | 1.00 | 56.09 |
| 1824 | CB | LYS | A | 447 | 70.763 | −12.215 | 91.401 | 1.00 | 49.97 |
| 1825 | CG | LYS | A | 447 | 71.562 | −11.250 | 90.572 | 1.00 | 55.67 |
| 1826 | CD | LYS | A | 447 | 71.274 | −11.363 | 89.065 | 1.00 | 63.06 |
| 1827 | CE | LYS | A | 447 | 72.560 | −11.557 | 88.271 | 1.00 | 63.18 |
| 1828 | NZ | LYS | A | 447 | 73.105 | −12.933 | 88.506 | 1.00 | 72.36 |
| 1829 | C | LYS | A | 447 | 70.743 | −12.903 | 93.822 | 1.00 | 53.51 |
| 1830 | O | LYS | A | 447 | 69.635 | −13.451 | 93.839 | 1.00 | 52.52 |
| 1831 | N | LYS | A | 448 | 71.304 | −12.377 | 94.907 | 1.00 | 49.20 |
| 1832 | CA | LYS | A | 448 | 70.604 | −12.378 | 96.169 | 1.00 | 47.16 |
| 1833 | CB | LYS | A | 448 | 71.445 | −13.071 | 97.233 | 1.00 | 49.48 |
| 1834 | CG | LYS | A | 448 | 71.046 | −14.549 | 97.444 | 1.00 | 62.61 |
| 1835 | CD | LYS | A | 448 | 69.851 | −14.716 | 98.413 | 1.00 | 67.54 |
| 1836 | CE | LYS | A | 448 | 68.552 | −14.056 | 97.904 | 1.00 | 66.83 |
| 1837 | NZ | LYS | A | 448 | 68.053 | −13.052 | 98.888 | 1.00 | 63.74 |
| 1838 | C | LYS | A | 448 | 70.210 | −10.972 | 96.600 | 1.00 | 46.23 |
| 1839 | O | LYS | A | 448 | 70.942 | −9.999 | 96.385 | 1.00 | 46.82 |
| 1840 | N | LEU | A | 449 | 69.025 | −10.880 | 97.202 | 1.00 | 42.49 |
| 1841 | CA | LEU | A | 449 | 68.477 | −9.606 | 97.658 | 1.00 | 38.18 |
| 1842 | CB | LEU | A | 449 | 66.964 | −9.753 | 97.815 | 1.00 | 33.91 |
| 1843 | CG | LEU | A | 449 | 66.269 | −10.457 | 96.646 | 1.00 | 36.17 |
| 1844 | CD1 | LEU | A | 449 | 64.771 | −10.696 | 96.955 | 1.00 | 10.94 |
| 1845 | CD2 | LEU | A | 449 | 66.458 | −9.591 | 95.389 | 1.00 | 35.40 |
| 1846 | C | LEU | A | 449 | 69.084 | −9.118 | 98.969 | 1.00 | 34.08 |
| 1847 | O | LEU | A | 449 | 69.184 | −9.875 | 99.912 | 1.00 | 40.43 |
| 1848 | N | PRO | A | 450 | 69.530 | −7.857 | 99.034 | 1.00 | 28.11 |
| 1849 | CD | PRO | A | 450 | 69.629 | −6.817 | 98.006 | 1.00 | 21.01 |
| 1850 | CA | PRO | A | 450 | 70.097 | −7.415 | 100.312 | 1.00 | 27.75 |
| 1851 | CB | PRO | A | 450 | 70.333 | −5.933 | 100.116 | 1.00 | 21.74 |
| 1852 | CG | PRO | A | 450 | 69.528 | −5.608 | 98.846 | 1.00 | 26.89 |
| 1853 | C | PRO | A | 450 | 69.061 | −7.665 | 101.374 | 1.00 | 30.49 |
| 1854 | O | PRO | A | 450 | 67.858 | −7.637 | 101.071 | 1.00 | 28.32 |
| 1855 | N | PRO | A | 451 | 69.504 | −7.909 | 102.631 | 1.00 | 32.59 |
| 1856 | CD | PRO | A | 451 | 70.885 | −7.627 | 103.044 | 1.00 | 28.81 |
| 1857 | CA | PRO | A | 451 | 68.680 | −8.187 | 103.821 | 1.00 | 32.25 |
| 1858 | CB | PRO | A | 451 | 69.584 | −7.758 | 104.992 | 1.00 | 24.82 |
| 1859 | CG | PRO | A | 451 | 70.664 | −6.951 | 104.345 | 1.00 | 23.17 |
| 1860 | C | PRO | A | 451 | 67.274 | −7.569 | 103.909 | 1.00 | 30.85 |
| 1861 | O | PRO | A | 451 | 66.282 | −8.303 | 104.031 | 1.00 | 33.81 |
| 1862 | N | LEU | A | 452 | 67.177 | −6.245 | 103.862 | 1.00 | 24.53 |
| 1863 | CA | LEU | A | 452 | 65.863 | −5.616 | 103.951 | 1.00 | 27.08 |
| 1864 | CB | LEU | A | 452 | 65.978 | −4.111 | 103.775 | 1.00 | 30.47 |
| 1865 | CG | LEU | A | 452 | 64.687 | −3.314 | 103.885 | 1.00 | 21.32 |
| 1866 | CD1 | LEU | A | 452 | 64.116 | −3.523 | 105.260 | 1.00 | 27.16 |
| 1867 | CD2 | LEU | A | 452 | 64.959 | −1.820 | 103.642 | 1.00 | 23.69 |
| 1868 | C | LEU | A | 452 | 64.839 | −6.149 | 102.940 | 1.00 | 26.24 |
| 1869 | O | LEU | A | 452 | 63.734 | −6.521 | 103.327 | 1.00 | 25.87 |
| 1870 | N | LEU | A | 453 | 65.206 | −6.176 | 101.657 | 1.00 | 25.20 |
| 1871 | CA | LEU | A | 453 | 64.325 | −6.665 | 100.605 | 1.00 | 24.92 |
| 1872 | CB | LEU | A | 453 | 64.973 | −6.487 | 99.234 | 1.00 | 19.86 |
| 1873 | CG | LEU | A | 453 | 65.268 | −5.025 | 98.933 | 1.00 | 21.10 |
| 1874 | CD1 | LEU | A | 453 | 65.936 | −4.836 | 97.561 | 1.00 | 17.34 |
| 1875 | CD2 | LEU | A | 453 | 63.942 | −4.295 | 98.975 | 1.00 | 10.81 |
| 1876 | C | LEU | A | 453 | 64.037 | −8.125 | 100.835 | 1.00 | 26.37 |
| 1877 | O | LEU | A | 453 | 62.884 | −8.565 | 100.836 | 1.00 | 28.39 |
| 1878 | N | SER | A | 454 | 65.110 | −8.880 | 101.024 | 1.00 | 29.03 |
| 1879 | CA | SER | A | 454 | 65.013 | −10.315 | 101.266 | 1.00 | 27.64 |
| 1880 | CB | SER | A | 454 | 66.397 | −10.859 | 101.638 | 1.00 | 20.09 |
| 1881 | OG | SER | A | 454 | 66.275 | −12.168 | 102.148 | 1.00 | 34.94 |
| 1882 | C | SER | A | 454 | 64.004 | −10.660 | 102.377 | 1.00 | 23.26 |
| 1883 | O | SER | A | 454 | 63.401 | −11.723 | 102.368 | 1.00 | 22.09 |
| 1884 | N | GLU | A | 455 | 63.828 | −9.743 | 103.321 | 1.00 | 19.56 |
| 1885 | CA | GLU | A | 455 | 62.935 | −9.975 | 104.428 | 1.00 | 20.53 |
| 1886 | CB | GLU | A | 455 | 63.162 | −8.919 | 105.473 | 1.00 | 17.19 |
| 1887 | CG | GLU | A | 455 | 62.467 | −9.149 | 106.738 | 1.00 | 28.76 |
| 1888 | CD | GLU | A | 455 | 62.962 | −8.167 | 107.813 | 1.00 | 36.58 |
| 1889 | OE1 | GLU | A | 455 | 64.138 | −8.236 | 108.230 | 1.00 | 34.71 |
| 1890 | OE2 | GLU | A | 455 | 62.187 | −7.292 | 108.241 | 1.00 | 34.84 |
| 1891 | C | GLU | A | 455 | 61.523 | −9.945 | 103.955 | 1.00 | 24.54 |
| 1892 | O | GLU | A | 455 | 60.768 | −10.879 | 104.206 | 1.00 | 25.12 |
| 1893 | N | ILE | A | 456 | 61.187 | −8.880 | 103.229 | 1.00 | 28.65 |
| 1894 | CA | ILE | A | 456 | 59.847 | −8.657 | 102.688 | 1.00 | 28.59 |
| 1895 | CB | ILE | A | 456 | 59.691 | −7.181 | 102.223 | 1.00 | 35.98 |
| 1896 | CG2 | ILE | A | 456 | 58.287 | −6.906 | 101.712 | 1.00 | 44.98 |
| 1897 | CG1 | ILE | A | 456 | 59.972 | −6.255 | 103.391 | 1.00 | 39.73 |
| 1898 | CD1 | ILE | A | 456 | 60.085 | −4.841 | 102.959 | 1.00 | 48.93 |
| 1899 | C | ILE | A | 456 | 59.473 | −9.527 | 101.524 | 1.00 | 24.83 |
| 1900 | O | ILE | A | 456 | 58.292 | −9.774 | 101.305 | 1.00 | 23.51 |
| 1901 | N | TRP | A | 457 | 60.480 | −10.011 | 100.801 | 1.00 | 26.84 |
| 1902 | CA | TRP | A | 457 | 60.237 | −10.789 | 99.594 | 1.00 | 30.79 |
| 1903 | CB | TRP | A | 457 | 60.793 | −10.025 | 98.378 | 1.00 | 30.70 |
| 1904 | CG | TRP | A | 457 | 60.180 | −8.671 | 98.224 | 1.00 | 26.75 |
| 1905 | CD2 | TRP | A | 457 | 60.724 | −7.544 | 97.533 | 1.00 | 25.63 |
| 1906 | CE2 | TRP | A | 457 | 59.788 | −6.485 | 97.660 | 1.00 | 28.89 |
| 1907 | CE3 | TRP | A | 457 | 61.914 | −7.313 | 96.836 | 1.00 | 17.92 |
| 1908 | CD1 | TRP | A | 457 | 58.966 | −8.270 | 98.716 | 1.00 | 21.14 |
| 1909 | NE1 | TRP | A | 457 | 58.723 | −6.971 | 98.383 | 1.00 | 16.57 |
| 1910 | CZ2 | TRP | A | 457 | 59.992 | −5.216 | 97.095 | 1.00 | 23.51 |
| 1911 | CZ3 | TRP | A | 457 | 62.112 | −6.066 | 96.283 | 1.00 | 27.28 |
| 1912 | CH2 | TRP | A | 457 | 61.152 | −5.024 | 96.423 | 1.00 | 22.51 |
| 1913 | C | TRP | A | 457 | 60.710 | −12.214 | 99.538 | 1.00 | 31.41 |
| 1914 | O | TRP | A | 457 | 60.088 | −13.063 | 98.892 | 1.00 | 29.63 |
| 1915 | N | ASP | A | 458 | 61.818 | −12.479 | 100.197 | 1.00 | 32.34 |
| 1916 | CA | ASP | A | 458 | 62.337 | −13.814 | 100.159 | 1.00 | 35.03 |
| 1917 | CB | ASP | A | 458 | 63.768 | −13.824 | 100.670 | 1.00 | 34.90 |
| 1918 | CG | ASP | A | 458 | 64.769 | −13.691 | 99.569 | 1.00 | 38.51 |
| 1919 | OD1 | ASP | A | 458 | 64.570 | −14.363 | 98.521 | 1.00 | 43.44 |
| 1920 | OD2 | ASP | A | 458 | 65.762 | −12.945 | 99.769 | 1.00 | 42.94 |
| 1921 | C | ASP | A | 458 | 61.482 | −14.771 | 100.971 | 1.00 | 40.93 |
| 1922 | O | ASP | A | 458 | 60.890 | −14.395 | 101.989 | 1.00 | 41.04 |
| 1923 | N | VAL | A | 459 | 61.392 | −16.012 | 100.502 | 1.00 | 47.90 |
| 1924 | CA | VAL | A | 459 | 60.634 | −17.040 | 101.215 | 1.00 | 51.13 |
| 1925 | CB | VAL | A | 459 | 60.116 | −18.089 | 100.266 | 1.00 | 36.52 |
| 1926 | CG1 | VAL | A | 459 | 59.392 | −19.139 | 101.029 | 1.00 | 46.60 |
| 1927 | CG2 | VAL | A | 459 | 59.202 | −17.439 | 99.278 | 1.00 | 44.26 |
| 1928 | C | VAL | A | 459 | 61.599 | −17.699 | 102.193 | 1.00 | 58.53 |
| 1929 | O | VAL | A | 459 | 62.471 | −18.482 | 101.794 | 1.00 | 60.86 |
| 1930 | N | HIS | A | 460 | 61.454 | −17.382 | 103.472 | 1.00 | 65.09 |
| 1931 | CA | HIS | A | 460 | 62.350 | −17.948 | 104.474 | 1.00 | 75.10 |
| 1932 | CB | HIS | A | 460 | 63.470 | −16.945 | 104.783 | 1.00 | 78.97 |
| 1933 | CG | HIS | A | 460 | 62.963 | −15.598 | 105.207 | 1.00 | 83.29 |
| 1934 | CD2 | HIS | A | 460 | 63.001 | −14.961 | 106.405 | 1.00 | 85.25 |
| 1935 | ND1 | HIS | A | 460 | 62.268 | −14.759 | 104.360 | 1.00 | 84.43 |
| 1936 | CE1 | HIS | A | 460 | 61.898 | −13.670 | 105.011 | 1.00 | 78.08 |
| 1937 | NE2 | HIS | A | 460 | 62.331 | −13.768 | 106.256 | 1.00 | 85.09 |
| 1938 | C | HIS | A | 460 | 61.595 | −18.282 | 105.751 | 1.00 | 77.65 |
| 1939 | O | HIS | A | 460 | 60.932 | −17.394 | 106.305 | 1.00 | 80.63 |
| 1940 | N | ALA | A | 461 | 61.701 | −19.539 | 106.205 | 1.00 | 76.94 |
| 1941 | CA | ALA | A | 461 | 61.032 | −19.991 | 107.429 | 1.00 | 77.86 |
| 1942 | CB | ALA | A | 461 | 62.036 | −20.677 | 108.335 | 1.00 | 68.64 |
| 1943 | C | ALA | A | 461 | 60.326 | −18.851 | 108.198 | 1.00 | 81.03 |
| 1944 | O | ALA | A | 461 | 60.979 | −17.817 | 108.514 | 1.00 | 84.40 |
| 1945 | OT | ALA | A | 461 | 59.108 | −18.987 | 108.475 | 1.00 | 81.32 |
| 1946 | | ALA | A | 461 | | | | | |
| 1947 | CB | ALA | B | 218 | 19.034 | 7.478 | 89.070 | 1.00 | 43.15 |
| 1948 | C | ALA | B | 218 | 17.931 | 5.243 | 89.097 | 1.00 | 46.46 |
| 1949 | O | ALA | B | 218 | 18.818 | 4.400 | 89.085 | 1.00 | 52.84 |
| 1950 | N | ALA | B | 218 | 18.545 | 6.261 | 91.233 | 1.00 | 43.35 |
| 1951 | CA | ALA | B | 218 | 18.073 | 6.541 | 89.839 | 1.00 | 47.62 |
| 1952 | N | ALA | B | 219 | 16.788 | 5.128 | 88.454 | 1.00 | 44.52 |
| 1953 | CA | ALA | B | 219 | 16.409 | 3.957 | 87.713 | 1.00 | 41.03 |
| 1954 | CB | ALA | B | 219 | 14.932 | 4.005 | 87.422 | 1.00 | 34.92 |
| 1955 | C | ALA | B | 219 | 17.137 | 3.602 | 86.440 | 1.00 | 41.94 |
| 1956 | O | ALA | B | 219 | 17.480 | 4.420 | 85.616 | 1.00 | 44.70 |
| 1957 | N | LEU | B | 220 | 17.324 | 2.297 | 86.334 | 1.00 | 42.54 |
| 1958 | CA | LEU | B | 220 | 17.916 | 1.521 | 85.247 | 1.00 | 34.74 |
| 1959 | CB | LEU | B | 220 | 17.599 | 0.059 | 85.510 | 1.00 | 42.30 |
| 1960 | CG | LEU | B | 220 | 18.568 | −0.758 | 86.318 | 1.00 | 37.19 |
| 1961 | CD1 | LEU | B | 220 | 18.090 | −2.155 | 86.542 | 1.00 | 46.36 |
| 1962 | CD2 | LEU | B | 220 | 19.816 | −0.778 | 85.512 | 1.00 | 47.29 |
| 1963 | C | LEU | B | 220 | 17.235 | 1.846 | 83.934 | 1.00 | 32.94 |
| 1964 | O | LEU | B | 220 | 16.066 | 2.210 | 83.879 | 1.00 | 27.71 |
| 1965 | N | THR | B | 221 | 17.947 | 1.664 | 82.847 | 1.00 | 31.90 |
| 1966 | CA | THR | B | 221 | 17.305 | 1.913 | 81.579 | 1.00 | 31.38 |
| 1967 | CB | THR | B | 221 | 18.289 | 2.280 | 80.478 | 1.00 | 27.57 |
| 1968 | OG1 | THR | B | 221 | 19.084 | 1.137 | 80.152 | 1.00 | 29.79 |
| 1969 | CG2 | THR | B | 221 | 19.160 | 3.402 | 80.917 | 1.00 | 17.32 |
| 1970 | C | THR | B | 221 | 16.660 | 0.565 | 81.201 | 1.00 | 33.47 |
| 1971 | O | THR | B | 221 | 16.822 | −0.457 | 81.904 | 1.00 | 39.22 |
| 1972 | N | ALA | B | 222 | 15.915 | 0.569 | 80.103 | 1.00 | 27.17 |
| 1973 | CA | ALA | B | 222 | 15.240 | −0.625 | 79.640 | 1.00 | 25.42 |
| 1974 | CB | ALA | B | 222 | 14.261 | −0.253 | 78.495 | 1.00 | 10.46 |
| 1975 | C | ALA | B | 222 | 16.306 | −1.579 | 79.145 | 1.00 | 23.41 |
| 1976 | O | ALA | B | 222 | 16.246 | −2.793 | 79.414 | 1.00 | 20.42 |
| 1977 | N | ALA | B | 223 | 17.274 | −1.013 | 78.424 | 1.00 | 20.88 |
| 1978 | CA | ALA | B | 223 | 18.341 | −1.809 | 77.874 | 1.00 | 24.15 |
| 1979 | CB | ALA | B | 223 | 19.287 | −0.931 | 77.082 | 1.00 | 20.53 |
| 1980 | C | ALA | B | 223 | 19.083 | −2.542 | 78.993 | 1.00 | 28.88 |
| 1981 | O | ALA | B | 223 | 19.419 | −3.748 | 78.865 | 1.00 | 28.82 |
| 1982 | N | GLN | B | 224 | 19.334 | −1.826 | 80.093 | 1.00 | 29.60 |
| 1983 | CA | GLN | B | 224 | 20.056 | −2.423 | 81.211 | 1.00 | 31.76 |
| 1984 | CB | GLN | B | 224 | 20.563 | −1.320 | 82.158 | 1.00 | 27.51 |
| 1985 | CG | GLN | B | 224 | 21.613 | −0.405 | 81.584 | 1.00 | 29.93 |
| 1986 | CD | GLN | B | 224 | 22.080 | 0.627 | 82.596 | 1.00 | 33.83 |
| 1987 | OE1 | GLN | B | 224 | 21.263 | 1.292 | 83.253 | 1.00 | 25.53 |
| 1988 | NE2 | GLN | B | 224 | 23.397 | 0.779 | 82.719 | 1.00 | 28.08 |
| 1989 | C | GLN | B | 224 | 19.207 | −3.461 | 81.983 | 1.00 | 30.31 |
| 1990 | O | GLN | B | 224 | 19.723 | −4.440 | 82.527 | 1.00 | 29.42 |
| 1991 | N | GLU | B | 225 | 17.906 | −3.239 | 82.026 | 1.00 | 29.37 |
| 1992 | CA | GLU | B | 225 | 17.048 | −4.150 | 82.720 | 1.00 | 30.65 |
| 1993 | CB | GLU | B | 225 | 15.651 | −3.573 | 82.868 | 1.00 | 39.85 |
| 1994 | CG | GLU | B | 225 | 14.883 | −4.208 | 84.022 | 1.00 | 48.26 |
| 1995 | CD | GLU | B | 225 | 13.600 | −3.484 | 84.317 | 1.00 | 56.77 |
| 1996 | OE1 | GLU | B | 225 | 13.649 | −2.238 | 84.448 | 1.00 | 65.13 |
| 1997 | OE2 | GLU | B | 225 | 12.552 | −4.160 | 84.418 | 1.00 | 49.77 |
| 1998 | C | GLU | B | 225 | 16.993 | −5.440 | 81.943 | 1.00 | 29.19 |
| 1999 | O | GLU | B | 225 | 17.083 | −6.522 | 82.538 | 1.00 | 31.21 |
| 2000 | N | LEU | B | 226 | 16.850 | −5.335 | 80.621 | 1.00 | 26.33 |
| 2001 | CA | LEU | B | 226 | 16.807 | −6.527 | 79.779 | 1.00 | 27.01 |
| 2002 | CB | LEU | B | 226 | 16.699 | −6.185 | 78.302 | 1.00 | 23.38 |
| 2003 | CG | LEU | B | 226 | 15.535 | −6.924 | 77.627 | 1.00 | 31.95 |
| 2004 | CD1 | LEU | B | 226 | 15.517 | −8.381 | 78.105 | 1.00 | 34.89 |
| 2005 | CD2 | LEU | B | 226 | 14.186 | −6.253 | 78.003 | 1.00 | 29.33 |
| 2006 | C | LEU | B | 226 | 18.054 | −7.349 | 79.967 | 1.00 | 28.78 |
| 2007 | O | LEU | B | 226 | 17.977 | −8.537 | 80.302 | 1.00 | 32.80 |
| 2008 | N | MET | B | 227 | 19.201 | −6.702 | 79.771 | 1.00 | 27.80 |
| 2009 | CA | MET | B | 227 | 20.515 | −7.342 | 79.898 | 1.00 | 27.07 |
| 2010 | CB | MET | B | 227 | 21.590 | −6.285 | 79.727 | 1.00 | 22.08 |
| 2011 | CG | MET | B | 227 | 22.970 | −6.838 | 79.790 | 1.00 | 30.34 |
| 2012 | SD | MET | B | 227 | 23.710 | −6.385 | 81.297 | 1.00 | 33.46 |
| 2013 | CE | MET | B | 227 | 23.478 | −4.568 | 81.248 | 1.00 | 42.18 |
| 2014 | C | MET | B | 227 | 20.733 | −8.099 | 81.212 | 1.00 | 26.99 |
| 2015 | O | MET | B | 227 | 21.194 | −9.240 | 81.231 | 1.00 | 27.73 |
| 2016 | N | ILE | B | 228 | 20.393 | −7.444 | 82.312 | 1.00 | 26.23 |
| 2017 | CA | ILE | B | 228 | 20.523 | −8.043 | 83.633 | 1.00 | 21.67 |
| 2018 | CB | ILE | B | 228 | 20.181 | −7.039 | 84.776 | 1.00 | 18.41 |
| 2019 | CG2 | ILE | B | 228 | 20.292 | −7.744 | 86.087 | 1.00 | 14.71 |
| 2020 | CG1 | ILE | B | 228 | 21.122 | −5.825 | 84.734 | 1.00 | 11.85 |
| 2021 | CD1 | ILE | B | 228 | 20.822 | −4.797 | 85.744 | 1.00 | 5.36 |
| 2022 | C | ILE | B | 228 | 19.582 | −9.227 | 83.758 | 1.00 | 19.46 |
| 2023 | O | ILE | B | 228 | 19.979 | −10.299 | 84.207 | 1.00 | 13.61 |
| 2024 | N | GLN | B | 229 | 18.336 | −9.025 | 83.344 | 1.00 | 23.63 |
| 2025 | CA | GLN | B | 229 | 17.327 | −10.080 | 83.409 | 1.00 | 28.46 |
| 2026 | CB | GLN | B | 229 | 15.990 | −9.587 | 82.881 | 1.00 | 28.33 |
| 2027 | CG | GLN | B | 229 | 14.828 | −10.407 | 83.399 | 1.00 | 38.50 |
| 2028 | CD | GLN | B | 229 | 13.560 | −10.231 | 82.552 | 1.00 | 52.93 |
| 2029 | OE1 | GLN | B | 229 | 13.351 | −10.913 | 81.513 | 1.00 | 43.06 |
| 2030 | NE2 | GLN | B | 229 | 12.713 | −9.303 | 82.982 | 1.00 | 52.10 |
| 2031 | C | GLN | B | 229 | 17.764 | −11.292 | 82.613 | 1.00 | 28.34 |
| 2032 | O | GLN | B | 229 | 17.600 | −12.432 | 83.064 | 1.00 | 31.45 |
| 2033 | N | GLN | B | 230 | 18.352 | −11.041 | 81.449 | 1.00 | 28.55 |
| 2034 | CA | GLN | B | 230 | 18.840 | −12.110 | 80.575 | 1.00 | 33.32 |
| 2035 | CB | GLN | B | 230 | 19.151 | −11.527 | 79.203 | 1.00 | 40.65 |
| 2036 | CG | GLN | B | 230 | 17.968 | −10.761 | 78.647 | 1.00 | 51.44 |
| 2037 | CD | GLN | B | 230 | 18.292 | −10.024 | 77.374 | 1.00 | 60.22 |
| 2038 | OE1 | GLN | B | 230 | 19.391 | −9.433 | 77.211 | 1.00 | 66.06 |
| 2039 | NE2 | GLN | B | 230 | 17.330 | −10.034 | 76.452 | 1.00 | 56.15 |
| 2040 | C | GLN | B | 230 | 20.081 | −12.839 | 81.118 | 1.00 | 34.16 |
| 2041 | O | GLN | B | 230 | 20.192 | −14.061 | 81.000 | 1.00 | 34.60 |
| 2042 | N | LEU | B | 231 | 21.022 | −12.086 | 81.690 | 1.00 | 33.62 |
| 2043 | CA | LEU | B | 231 | 22.232 | −12.672 | 82.247 | 1.00 | 27.03 |
| 2044 | CB | LEU | B | 231 | 23.105 | −11.596 | 82.862 | 1.00 | 24.13 |
| 2045 | CG | LEU | B | 231 | 24.142 | −10.884 | 82.027 | 1.00 | 30.54 |
| 2046 | CD1 | LEU | B | 231 | 25.107 | −10.252 | 83.033 | 1.00 | 39.41 |
| 2047 | CD2 | LEU | B | 231 | 24.901 | −11.854 | 81.118 | 1.00 | 21.02 |
| 2048 | C | LEU | B | 231 | 21.838 | −13.656 | 83.331 | 1.00 | 25.58 |
| 2049 | O | LEU | B | 231 | 22.368 | −14.754 | 83.421 | 1.00 | 24.70 |
| 2050 | N | VAL | B | 232 | 20.901 | −13.252 | 84.172 | 1.00 | 23.19 |
| 2051 | CA | VAL | B | 232 | 20.478 | −14.123 | 85.237 | 1.00 | 20.58 |
| 2052 | CB | VAL | B | 232 | 19.534 | −13.397 | 86.181 | 1.00 | 19.93 |
| 2053 | CG1 | VAL | B | 232 | 18.784 | −14.414 | 87.042 | 1.00 | 6.79 |
| 2054 | CG2 | VAL | B | 232 | 20.365 | −12.359 | 87.027 | 1.00 | 5.25 |
| 2055 | C | VAL | B | 232 | 19.809 | −15.346 | 84.658 | 1.00 | 20.54 |
| 2056 | O | VAL | B | 232 | 20.014 | −16.461 | 85.156 | 1.00 | 24.31 |
| 2057 | N | ALA | B | 233 | 19.013 | −15.145 | 83.609 | 1.00 | 18.87 |
| 2058 | CA | ALA | B | 233 | 18.334 | −16.272 | 82.970 | 1.00 | 17.83 |
| 2059 | CB | ALA | B | 233 | 17.430 | −15.786 | 81.857 | 1.00 | 13.61 |
| 2060 | C | ALA | B | 233 | 19.361 | −17.247 | 82.416 | 1.00 | 15.43 |
| 2061 | O | ALA | B | 233 | 19.247 | −18.457 | 82.583 | 1.00 | 15.77 |
| 2062 | N | ALA | B | 234 | 20.375 | −16.701 | 81.763 | 1.00 | 19.63 |
| 2063 | CA | ALA | B | 234 | 21.435 | −17.500 | 81.165 | 1.00 | 23.28 |
| 2064 | CB | ALA | B | 234 | 22.367 | −16.612 | 80.382 | 1.00 | 17.57 |
| 2065 | C | ALA | B | 234 | 22.207 | −18.240 | 82.254 | 1.00 | 27.69 |
| 2066 | O | ALA | B | 234 | 22.658 | −19.372 | 82.050 | 1.00 | 34.29 |
| 2067 | N | GLN | B | 235 | 22.347 | −17.626 | 83.421 | 1.00 | 24.90 |
| 2068 | CA | GLN | B | 235 | 23.075 | −18.279 | 84.484 | 1.00 | 26.11 |
| 2069 | CB | GLN | B | 235 | 23.219 | −17.344 | 85.662 | 1.00 | 30.42 |
| 2070 | CG | GLN | B | 235 | 24.135 | −17.845 | 86.735 | 1.00 | 42.13 |
| 2071 | CD | GLN | B | 235 | 25.458 | −17.067 | 86.789 | 1.00 | 50.21 |
| 2072 | OE1 | GLN | B | 235 | 25.674 | −16.205 | 87.680 | 1.00 | 33.87 |
| 2073 | NE2 | GLN | B | 235 | 26.348 | −17.351 | 85.819 | 1.00 | 46.88 |
| 2074 | C | GLN | B | 235 | 22.310 | −19.491 | 84.919 | 1.00 | 26.10 |
| 2075 | O | GLN | B | 235 | 22.888 | −20.519 | 85.235 | 1.00 | 27.29 |
| 2076 | N | LEU | B | 236 | 20.988 | −19.371 | 84.901 | 1.00 | 28.38 |
| 2077 | CA | LEU | B | 236 | 20.123 | −20.465 | 85.337 | 1.00 | 30.72 |
| 2078 | CB | LEU | B | 236 | 18.703 | −19.945 | 85.664 | 1.00 | 27.81 |
| 2079 | CG | LEU | B | 236 | 17.924 | −20.844 | 86.651 | 1.00 | 28.43 |
| 2080 | CD1 | LEU | B | 236 | 18.850 | −21.322 | 87.773 | 1.00 | 11.13 |
| 2081 | CD2 | LEU | B | 236 | 16.768 | −20.057 | 87.251 | 1.00 | 18.53 |
| 2082 | C | LEU | B | 236 | 20.043 | −21.610 | 84.335 | 1.00 | 30.32 |
| 2083 | O | LEU | B | 236 | 20.228 | −22.794 | 84.676 | 1.00 | 28.58 |
| 2084 | N | GLN | B | 237 | 19.783 | −21.250 | 83.091 | 1.00 | 26.79 |
| 2085 | CA | GLN | B | 237 | 19.687 | −22.249 | 82.069 | 1.00 | 28.98 |
| 2086 | CB | GLN | B | 237 | 19.259 | −21.576 | 80.760 | 1.00 | 35.06 |
| 2087 | CG | GLN | B | 237 | 17.742 | −21.445 | 80.658 | 1.00 | 47.36 |
| 2088 | CD | GLN | B | 237 | 17.128 | −22.745 | 80.202 | 1.00 | 53.79 |
| 2089 | OE1 | GLN | B | 237 | 16.011 | −23.102 | 80.584 | 1.00 | 60.26 |
| 2090 | NE2 | GLN | B | 237 | 17.872 | −23.473 | 79.366 | 1.00 | 42.25 |
| 2091 | C | GLN | B | 237 | 20.990 | −23.031 | 81.918 | 1.00 | 28.95 |
| 2092 | O | GLN | B | 237 | 20.979 | −24.268 | 81.847 | 1.00 | 28.13 |
| 2093 | N | CYS | B | 238 | 22.111 | −22.315 | 81.881 | 1.00 | 28.66 |
| 2094 | CA | CYS | B | 238 | 23.397 | −22.972 | 81.729 | 1.00 | 29.72 |
| 2095 | CB | CYS | B | 238 | 24.507 | −21.942 | 81.564 | 1.00 | 34.27 |
| 2096 | SG | CYS | B | 238 | 24.593 | −21.236 | 79.948 | 1.00 | 27.05 |
| 2097 | C | CYS | B | 238 | 23.680 | −23.845 | 82.933 | 1.00 | 26.51 |
| 2098 | O | CYS | B | 238 | 24.299 | −24.913 | 82.819 | 1.00 | 24.18 |
| 2099 | N | ASN | B | 239 | 23.204 | −23.392 | 84.083 | 1.00 | 24.48 |
| 2100 | CA | ASN | B | 239 | 23.413 | −24.149 | 85.290 | 1.00 | 28.69 |
| 2101 | CB | ASN | B | 239 | 23.089 | −23.285 | 86.518 | 1.00 | 25.28 |
| 2102 | CG | ASN | B | 239 | 23.332 | −24.028 | 87.820 | 1.00 | 29.79 |
| 2103 | OD1 | ASN | B | 239 | 24.423 | −24.519 | 88.053 | 1.00 | 32.08 |
| 2104 | ND2 | ASN | B | 239 | 22.311 | −24.119 | 88.669 | 1.00 | 42.98 |
| 2105 | C | ASN | B | 239 | 22.548 | −25.430 | 85.274 | 1.00 | 31.07 |
| 2106 | O | ASN | B | 239 | 22.998 | −26.550 | 85.621 | 1.00 | 29.58 |
| 2107 | N | ALA | B | 240 | 21.303 | −25.263 | 84.846 | 1.00 | 31.87 |
| 2108 | CA | ALA | B | 240 | 20.380 | −26.392 | 84.803 | 1.00 | 32.86 |
| 2109 | CB | ALA | B | 240 | 18.951 | −25.912 | 84.500 | 1.00 | 19.97 |
| 2110 | C | ALA | B | 240 | 20.833 | −27.385 | 83.756 | 1.00 | 35.15 |
| 2111 | O | ALA | B | 240 | 20.611 | −28.567 | 83.881 | 1.00 | 38.25 |
| 2112 | N | ARG | B | 241 | 21.495 | −26.886 | 82.724 | 1.00 | 38.35 |
| 2113 | CA | ARG | B | 241 | 21.977 | −27.692 | 81.605 | 1.00 | 37.53 |
| 2114 | CB | ARG | B | 241 | 22.541 | −26.766 | 80.560 | 1.00 | 37.26 |
| 2115 | CG | ARG | B | 241 | 23.010 | −27.420 | 79.286 | 1.00 | 40.31 |
| 2116 | CD | ARG | B | 241 | 22.676 | −26.557 | 78.049 | 1.00 | 48.97 |
| 2117 | NE | ARG | B | 241 | 22.668 | −25.105 | 78.299 | 1.00 | 59.22 |
| 2118 | CZ | ARG | B | 241 | 21.778 | −24.271 | 77.761 | 1.00 | 58.75 |
| 2119 | NH1 | ARG | B | 241 | 20.858 | −24.767 | 76.952 | 1.00 | 58.70 |
| 2120 | NH2 | ARG | B | 241 | 21.770 | −22.967 | 78.066 | 1.00 | 53.27 |
| 2121 | C | ARG | B | 241 | 23.047 | −28.699 | 81.959 | 1.00 | 39.86 |
| 2122 | O | ARG | B | 241 | 23.026 | −29.840 | 81.534 | 1.00 | 42.08 |
| 2123 | N | SER | B | 242 | 23.981 | −28.259 | 82.776 | 1.00 | 42.38 |
| 2124 | CA | SER | B | 242 | 25.109 | −29.069 | 83.161 | 1.00 | 39.72 |
| 2125 | CB | SER | B | 242 | 26.323 | −28.140 | 83.355 | 1.00 | 44.91 |
| 2126 | OG | SER | B | 242 | 26.690 | −27.421 | 82.175 | 1.00 | 39.22 |
| 2127 | C | SER | B | 242 | 24.904 | −29.917 | 84.394 | 1.00 | 38.57 |
| 2128 | O | SER | B | 242 | 25.572 | −30.929 | 84.543 | 1.00 | 31.83 |
| 2129 | N | PHE | B | 243 | 23.966 | −29.513 | 85.250 | 1.00 | 41.47 |
| 2130 | CA | PHE | B | 243 | 23.736 | −30.217 | 86.497 | 1.00 | 42.59 |
| 2131 | CB | PHE | B | 243 | 24.229 | −29.345 | 87.639 | 1.00 | 41.86 |
| 2132 | CG | PHE | B | 243 | 25.712 | −29.064 | 87.593 | 1.00 | 41.55 |
| 2133 | CD1 | PHE | B | 243 | 26.206 | −27.924 | 86.946 | 1.00 | 41.40 |
| 2134 | CD2 | PHE | B | 243 | 26.621 | −29.956 | 88.201 | 1.00 | 35.89 |
| 2135 | CE1 | PHE | B | 243 | 27.603 | −27.672 | 86.904 | 1.00 | 41.54 |
| 2136 | CE2 | PHE | B | 243 | 28.009 | −29.724 | 88.167 | 1.00 | 33.17 |
| 2137 | CZ | PHE | B | 243 | 28.501 | −28.575 | 87.515 | 1.00 | 44.01 |
| 2138 | C | PHE | B | 243 | 22.346 | −30.730 | 86.824 | 1.00 | 44.54 |
| 2139 | O | PHE | B | 243 | 22.173 | −31.408 | 87.815 | 1.00 | 49.89 |
| 2140 | N | SER | B | 244 | 21.355 | −30.398 | 86.013 | 1.00 | 45.17 |
| 2141 | CA | SER | B | 244 | 20.013 | −30.917 | 86.225 | 1.00 | 44.26 |
| 2142 | CB | SER | B | 244 | 19.172 | −30.724 | 84.975 | 1.00 | 48.33 |
| 2143 | OG | SER | B | 244 | 18.426 | −29.525 | 85.050 | 1.00 | 51.93 |
| 2144 | C | SER | B | 244 | 20.112 | −32.402 | 86.496 | 1.00 | 46.85 |
| 2145 | O | SER | B | 244 | 19.657 | −32.890 | 87.522 | 1.00 | 49.00 |
| 2146 | N | ASP | B | 245 | 20.717 | −33.106 | 85.550 | 1.00 | 49.90 |
| 2147 | CA | ASP | B | 245 | 20.938 | −34.530 | 85.655 | 1.00 | 52.24 |
| 2148 | CB | ASP | B | 245 | 21.253 | −35.097 | 84.272 | 1.00 | 61.31 |
| 2149 | CG | ASP | B | 245 | 20.013 | −35.253 | 83.453 | 1.00 | 73.36 |
| 2150 | OD1 | ASP | B | 245 | 18.925 | −35.317 | 84.092 | 1.00 | 81.72 |
| 2151 | OD2 | ASP | B | 245 | 20.118 | −35.316 | 82.203 | 1.00 | 79.76 |
| 2152 | C | ASP | B | 245 | 22.083 | −34.775 | 86.611 | 1.00 | 49.00 |
| 2153 | O | ASP | B | 245 | 23.127 | −34.151 | 86.512 | 1.00 | 50.73 |
| 2154 | N | GLN | B | 246 | 21.886 | −35.677 | 87.550 | 1.00 | 45.53 |
| 2155 | CA | GLN | B | 246 | 22.928 | −35.942 | 88.500 | 1.00 | 43.82 |
| 2156 | CB | GLN | B | 246 | 22.429 | −36.961 | 89.497 | 1.00 | 49.76 |
| 2157 | CG | GLN | B | 246 | 23.483 | −37.580 | 90.380 | 1.00 | 53.84 |
| 2158 | CD | GLN | B | 246 | 22.870 | −37.989 | 91.686 | 1.00 | 54.86 |
| 2159 | OE1 | GLN | B | 246 | 23.045 | −39.112 | 92.155 | 1.00 | 55.22 |
| 2160 | NE2 | GLN | B | 246 | 22.122 | −37.066 | 92.287 | 1.00 | 56.87 |
| 2161 | C | GLN | B | 246 | 24.203 | −36.419 | 87.837 | 1.00 | 43.71 |
| 2162 | O | GLN | B | 246 | 24.186 | −37.286 | 86.960 | 1.00 | 44.65 |
| 2163 | N | PRO | B | 247 | 25.341 | −35.853 | 88.252 | 1.00 | 43.54 |
| 2164 | CD | PRO | B | 247 | 25.529 | −34.846 | 89.315 | 1.00 | 43.31 |
| 2165 | CA | PRO | B | 247 | 26.611 | −36.269 | 87.651 | 1.00 | 43.57 |
| 2166 | CB | PRO | B | 247 | 27.665 | −35.411 | 88.352 | 1.00 | 43.02 |
| 2167 | CG | PRO | B | 247 | 26.919 | −34.345 | 89.074 | 1.00 | 41.73 |
| 2168 | C | PRO | B | 247 | 26.939 | −37.735 | 87.855 | 1.00 | 45.12 |
| 2169 | O | PRO | B | 247 | 26.759 | −38.268 | 88.954 | 1.00 | 44.37 |
| 2170 | N | LYS | B | 248 | 27.448 | −38.362 | 86.792 | 1.00 | 48.61 |
| 2171 | CA | LYS | B | 248 | 27.897 | −39.766 | 86.812 | 1.00 | 45.27 |
| 2172 | CB | LYS | B | 248 | 27.730 | −40.391 | 85.424 | 1.00 | 50.09 |
| 2173 | CG | LYS | B | 248 | 26.380 | −40.138 | 84.785 | 1.00 | 54.27 |
| 2174 | CD | LYS | B | 248 | 25.309 | −41.021 | 85.407 | 1.00 | 37.79 |
| 2175 | CE | LYS | B | 248 | 24.054 | −41.016 | 84.558 | 1.00 | 45.41 |
| 2176 | NZ | LYS | B | 248 | 23.084 | −41.991 | 85.091 | 1.00 | 48.02 |
| 2177 | C | LYS | B | 248 | 29.399 | −39.607 | 87.110 | 1.00 | 42.33 |
| 2178 | O | LYS | B | 248 | 30.246 | −39.782 | 86.250 | 1.00 | 47.14 |
| 2179 | N | VAL | B | 249 | 29.728 | −39.276 | 88.341 | 1.00 | 37.10 |
| 2180 | CA | VAL | B | 249 | 31.107 | −39.014 | 88.698 | 1.00 | 33.75 |
| 2181 | CB | VAL | B | 249 | 31.175 | −37.518 | 89.217 | 1.00 | 30.79 |
| 2182 | CG1 | VAL | B | 249 | 32.573 | −37.103 | 89.545 | 1.00 | 31.67 |
| 2183 | CG2 | VAL | B | 249 | 30.612 | −36.576 | 88.177 | 1.00 | 22.73 |
| 2184 | C | VAL | B | 249 | 31.652 | −39.991 | 89.765 | 1.00 | 34.87 |
| 2185 | O | VAL | B | 249 | 30.892 | −40.641 | 90.500 | 1.00 | 37.01 |
| 2186 | N | THR | B | 250 | 32.971 | −40.115 | 89.848 | 1.00 | 32.17 |
| 2187 | CA | THR | B | 250 | 33.555 | −40.935 | 90.902 | 1.00 | 31.48 |
| 2188 | CB | THR | B | 250 | 35.075 | −40.691 | 91.007 | 1.00 | 30.25 |
| 2189 | OG1 | THR | B | 250 | 35.711 | −41.205 | 89.831 | 1.00 | 38.00 |
| 2190 | CG2 | THR | B | 250 | 35.642 | −41.378 | 92.248 | 1.00 | 25.30 |
| 2191 | C | THR | B | 250 | 32.870 | −40.495 | 92.203 | 1.00 | 27.91 |
| 2192 | O | THR | B | 250 | 32.908 | −39.320 | 92.552 | 1.00 | 26.73 |
| 2193 | N | PRO | B | 251 | 32.213 | −41.425 | 92.922 | 1.00 | 28.52 |
| 2194 | CD | PRO | B | 251 | 32.263 | −42.887 | 92.717 | 1.00 | 29.08 |
| 2195 | CA | PRO | B | 251 | 31.518 | −41.087 | 94.184 | 1.00 | 28.02 |
| 2196 | CB | PRO | B | 251 | 30.795 | −42.379 | 94.532 | 1.00 | 25.83 |
| 2197 | CG | PRO | B | 251 | 31.771 | −43.428 | 94.058 | 1.00 | 30.66 |
| 2198 | C | PRO | B | 251 | 32.428 | −40.620 | 95.337 | 1.00 | 24.48 |
| 2199 | O | PRO | B | 251 | 33.609 | −41.024 | 95.405 | 1.00 | 23.99 |
| 2200 | N | TRP | B | 252 | 31.899 | −39.755 | 96.208 | 1.00 | 19.10 |
| 2201 | CA | TRP | B | 252 | 32.685 | −39.295 | 97.335 | 1.00 | 21.37 |
| 2202 | CB | TRP | B | 252 | 32.013 | −38.096 | 97.975 | 1.00 | 18.10 |
| 2203 | CG | TRP | B | 252 | 32.754 | −37.556 | 99.194 | 1.00 | 31.18 |
| 2204 | CD2 | TRP | B | 252 | 33.789 | −36.556 | 99.217 | 1.00 | 35.34 |
| 2205 | CE2 | TRP | B | 252 | 34.150 | −36.355 | 100.581 | 1.00 | 38.50 |
| 2206 | CE3 | TRP | B | 252 | 34.463 | −35.820 | 98.226 | 1.00 | 37.47 |
| 2207 | CD1 | TRP | B | 252 | 32.535 | −37.904 | 100.517 | 1.00 | 31.91 |
| 2208 | NE1 | TRP | B | 252 | 33.366 | −37.183 | 101.348 | 1.00 | 30.53 |
| 2209 | CZ2 | TRP | B | 252 | 35.139 | −35.424 | 100.969 | 1.00 | 33.94 |
| 2210 | CZ3 | TRP | B | 252 | 35.453 | −34.896 | 98.624 | 1.00 | 38.18 |
| 2211 | CH2 | TRP | B | 252 | 35.779 | −34.717 | 99.976 | 1.00 | 30.25 |
| 2212 | C | TRP | B | 252 | 32.802 | −40.479 | 98.313 | 1.00 | 27.08 |
| 2213 | O | TRP | B | 252 | 31.830 | −41.038 | 98.769 | 1.00 | 25.70 |
| 2214 | N | PRO | B | 253 | 34.012 | −40.872 | 98.653 | 1.00 | 32.95 |
| 2215 | CD | PRO | B | 253 | 35.349 | −40.373 | 98.286 | 1.00 | 42.21 |
| 2216 | CA | PRO | B | 253 | 34.082 | −42.010 | 99.566 | 1.00 | 39.27 |
| 2217 | CB | PRO | B | 253 | 35.532 | −42.483 | 99.409 | 1.00 | 36.35 |
| 2218 | CG | PRO | B | 253 | 36.272 | −41.159 | 99.242 | 1.00 | 46.96 |
| 2219 | C | PRO | B | 253 | 33.752 | −41.712 | 101.026 | 1.00 | 46.94 |
| 2220 | O | PRO | B | 253 | 33.999 | −40.605 | 101.516 | 1.00 | 49.86 |
| 2221 | N | LEU | B | 254 | 33.216 | −42.694 | 101.750 | 1.00 | 51.33 |
| 2222 | CA | LEU | B | 254 | 32.953 | −42.526 | 103.192 | 1.00 | 57.46 |
| 2223 | CB | LEU | B | 254 | 31.395 | −42.907 | 103.314 | 1.00 | 53.44 |
| 2224 | CG | LEU | B | 254 | 30.274 | −42.099 | 102.497 | 1.00 | 56.64 |
| 2225 | CD1 | LEU | B | 254 | 28.822 | −42.493 | 102.882 | 1.00 | 52.37 |
| 2226 | CD2 | LEU | B | 254 | 30.438 | −40.584 | 102.757 | 1.00 | 56.12 |
| 2227 | C | LEU | B | 254 | 34.020 | −43.213 | 104.160 | 1.00 | 60.78 |
| 2228 | O | LEU | B | 254 | 33.580 | −43.849 | 105.300 | 1.00 | 64.15 |
| 2229 | N | ALA | B | 258 | 35.369 | −42.937 | 103.834 | 1.00 | 65.16 |
| 2230 | CA | ALA | B | 258 | 36.450 | −43.716 | 104.419 | 1.00 | 66.69 |
| 2231 | CB | ALA | B | 258 | 36.889 | −44.471 | 103.293 | 1.00 | 72.53 |
| 2232 | C | ALA | B | 258 | 37.782 | −43.192 | 105.164 | 1.00 | 70.13 |
| 2233 | O | ALA | B | 258 | 37.780 | −42.100 | 105.635 | 1.00 | 71.40 |
| 2234 | N | GLN | B | 259 | 38.939 | −43.934 | 105.136 | 1.00 | 72.67 |
| 2235 | CA | GLN | B | 259 | 40.241 | −43.402 | 105.670 | 1.00 | 76.37 |
| 2236 | CB | GLN | B | 259 | 40.305 | −43.514 | 107.206 | 1.00 | 68.08 |
| 2237 | CG | GLN | B | 259 | 41.502 | −42.815 | 107.804 | 1.00 | 69.45 |
| 2238 | CD | GLN | B | 259 | 41.637 | −41.366 | 107.330 | 1.00 | 76.44 |
| 2239 | OE1 | GLN | B | 259 | 41.888 | −41.087 | 106.147 | 1.00 | 76.42 |
| 2240 | NE2 | GLN | B | 259 | 41.461 | −40.438 | 108.254 | 1.00 | 80.79 |
| 2241 | C | GLN | B | 259 | 41.473 | −44.121 | 105.020 | 1.00 | 79.69 |
| 2242 | O | GLN | B | 259 | 41.876 | −45.192 | 105.497 | 1.00 | 85.25 |
| 2243 | N | SER | B | 260 | 42.110 | −43.532 | 103.996 | 1.00 | 77.25 |
| 2244 | CA | SER | B | 260 | 43.195 | −44.247 | 103.321 | 1.00 | 74.33 |
| 2245 | CB | SER | B | 260 | 42.564 | −45.456 | 102.632 | 1.00 | 73.41 |
| 2246 | OG | SER | B | 260 | 43.486 | −46.188 | 101.864 | 1.00 | 85.05 |
| 2247 | C | SER | B | 260 | 43.921 | −43.391 | 102.287 | 1.00 | 74.57 |
| 2248 | O | SER | B | 260 | 43.700 | −42.186 | 102.196 | 1.00 | 72.81 |
| 2249 | N | ARG | B | 261 | 44.785 | −44.006 | 101.488 | 1.00 | 74.22 |
| 2250 | CA | ARG | B | 261 | 45.466 | −43.213 | 100.476 | 1.00 | 74.23 |
| 2251 | CB | ARG | B | 261 | 46.889 | −43.712 | 100.212 | 1.00 | 75.27 |
| 2252 | CG | ARG | B | 261 | 47.032 | −44.508 | 98.959 | 1.00 | 81.06 |
| 2253 | CD | ARG | B | 261 | 48.426 | −44.464 | 98.488 | 1.00 | 83.24 |
| 2254 | NE | ARG | B | 261 | 48.809 | −43.162 | 97.961 | 1.00 | 78.10 |
| 2255 | CZ | ARG | B | 261 | 49.819 | −43.022 | 97.121 | 1.00 | 74.06 |
| 2256 | NH1 | ARG | B | 261 | 50.477 | −44.107 | 96.756 | 1.00 | 72.36 |
| 2257 | NH2 | ARG | B | 261 | 50.181 | −41.823 | 96.678 | 1.00 | 73.29 |
| 2258 | C | ARG | B | 261 | 44.627 | −43.306 | 99.208 | 1.00 | 73.70 |
| 2259 | O | ARG | B | 261 | 44.635 | −42.398 | 98.374 | 1.00 | 75.08 |
| 2260 | N | ASP | B | 262 | 43.891 | −44.410 | 99.080 | 1.00 | 70.69 |
| 2261 | CA | ASP | B | 262 | 43.022 | −44.633 | 97.931 | 1.00 | 66.13 |
| 2262 | CB | ASP | B | 262 | 42.615 | −46.117 | 97.868 | 1.00 | 73.04 |
| 2263 | CG | ASP | B | 262 | 43.741 | −47.005 | 97.374 | 1.00 | 77.88 |
| 2264 | OD1 | ASP | B | 262 | 44.905 | −46.722 | 97.730 | 1.00 | 82.47 |
| 2265 | OD2 | ASP | B | 262 | 43.468 | −47.979 | 96.632 | 1.00 | 80.57 |
| 2266 | C | ASP | B | 262 | 41.805 | −43.738 | 98.101 | 1.00 | 59.93 |
| 2267 | O | ASP | B | 262 | 41.185 | −43.334 | 97.126 | 1.00 | 59.72 |
| 2268 | N | ALA | B | 263 | 41.494 | −43.413 | 99.352 | 1.00 | 52.51 |
| 2269 | CA | ALA | B | 263 | 40.366 | −42.561 | 99.652 | 1.00 | 47.78 |
| 2270 | CB | ALA | B | 263 | 40.012 | −42.646 | 101.127 | 1.00 | 48.71 |
| 2271 | C | ALA | B | 263 | 40.688 | −41.134 | 99.274 | 1.00 | 43.34 |
| 2272 | O | ALA | B | 263 | 39.812 | −40.389 | 98.828 | 1.00 | 41.68 |
| 2273 | N | ARG | B | 264 | 41.946 | −40.751 | 99.457 | 1.00 | 39.93 |
| 2274 | CA | ARG | B | 264 | 42.383 | −39.400 | 99.106 | 1.00 | 43.90 |
| 2275 | CB | ARG | B | 264 | 43.821 | −39.158 | 99.522 | 1.00 | 49.19 |
| 2276 | CG | ARG | B | 264 | 43.975 | −37.848 | 100.271 | 1.00 | 56.17 |
| 2277 | CD | ARG | B | 264 | 45.403 | −37.361 | 100.268 | 1.00 | 49.39 |
| 2278 | NE | ARG | B | 264 | 46.233 | −37.978 | 101.289 | 1.00 | 46.87 |
| 2279 | CZ | ARG | B | 264 | 47.402 | −38.513 | 101.019 | 1.00 | 56.69 |
| 2280 | NH1 | ARG | B | 264 | 47.826 | −38.500 | 99.767 | 1.00 | 62.48 |
| 2281 | NH2 | ARG | B | 264 | 48.149 | −39.018 | 101.996 | 1.00 | 66.89 |
| 2282 | C | ARG | B | 264 | 42.290 | −39.200 | 97.599 | 1.00 | 45.41 |
| 2283 | O | ARG | B | 264 | 41.719 | −38.220 | 97.100 | 1.00 | 46.21 |
| 2284 | N | GLN | B | 265 | 42.865 | −40.138 | 96.868 | 1.00 | 43.29 |
| 2285 | CA | GLN | B | 265 | 42.788 | −40.084 | 95.430 | 1.00 | 40.44 |
| 2286 | CB | GLN | B | 265 | 43.486 | −41.331 | 94.836 | 1.00 | 51.49 |
| 2287 | CG | GLN | B | 265 | 43.962 | −41.136 | 93.408 | 1.00 | 57.42 |
| 2288 | CD | GLN | B | 265 | 44.831 | −39.884 | 93.300 | 1.00 | 68.00 |
| 2289 | OE1 | GLN | B | 265 | 44.359 | −38.806 | 92.939 | 1.00 | 71.18 |
| 2290 | NE2 | GLN | B | 265 | 46.097 | −40.019 | 93.653 | 1.00 | 68.29 |
| 2291 | C | GLN | B | 265 | 41.296 | −40.032 | 94.968 | 1.00 | 38.02 |
| 2292 | O | GLN | B | 265 | 40.968 | −39.357 | 93.981 | 1.00 | 35.36 |
| 2293 | N | GLN | B | 266 | 40.398 | −40.737 | 95.661 | 1.00 | 30.69 |
| 2294 | CA | GLN | B | 266 | 39.022 | −40.754 | 95.227 | 1.00 | 27.06 |
| 2295 | CB | GLN | B | 266 | 38.235 | −41.787 | 95.990 | 1.00 | 28.96 |
| 2296 | CG | GLN | B | 266 | 37.447 | −42.711 | 95.111 | 1.00 | 23.71 |
| 2297 | CD | GLN | B | 266 | 36.420 | −43.468 | 95.896 | 1.00 | 21.89 |
| 2298 | OE1 | GLN | B | 266 | 35.372 | −42.935 | 96.183 | 1.00 | 21.16 |
| 2299 | NE2 | GLN | B | 266 | 36.722 | −44.717 | 96.260 | 1.00 | 30.55 |
| 2300 | C | GLN | B | 266 | 38.378 | −39.415 | 95.419 | 1.00 | 27.40 |
| 2301 | O | GLN | B | 266 | 37.747 | −38.876 | 94.487 | 1.00 | 29.30 |
| 2302 | N | ARG | B | 267 | 38.514 | −38.877 | 96.631 | 1.00 | 24.33 |
| 2303 | CA | ARG | B | 267 | 37.920 | −37.572 | 96.930 | 1.00 | 26.12 |
| 2304 | CB | ARG | B | 267 | 38.280 | −37.143 | 98.361 | 1.00 | 25.38 |
| 2305 | CG | ARG | B | 267 | 38.071 | −38.259 | 99.360 | 1.00 | 27.21 |
| 2306 | CD | ARG | B | 267 | 37.421 | −37.794 | 100.678 | 1.00 | 34.15 |
| 2307 | NE | ARG | B | 267 | 38.348 | −37.806 | 101.814 | 1.00 | 34.85 |
| 2308 | CZ | ARG | B | 267 | 38.780 | −38.907 | 102.420 | 1.00 | 36.34 |
| 2309 | NH1 | ARG | B | 267 | 38.363 | −40.098 | 102.011 | 1.00 | 45.28 |
| 2310 | NH2 | ARG | B | 267 | 39.658 | −38.828 | 103.409 | 1.00 | 32.14 |
| 2311 | C | ARG | B | 267 | 38.491 | −36.610 | 95.873 | 1.00 | 22.84 |
| 2312 | O | ARG | B | 267 | 37.790 | −35.806 | 95.269 | 1.00 | 15.70 |
| 2313 | N | PHE | B | 268 | 39.785 | −36.731 | 95.626 | 1.00 | 23.95 |
| 2314 | CA | PHE | B | 268 | 40.409 | −35.895 | 94.613 | 1.00 | 25.07 |
| 2315 | CB | PHE | B | 268 | 41.904 | −36.184 | 94.530 | 1.00 | 24.74 |
| 2316 | CG | PHE | B | 268 | 42.607 | −35.373 | 93.493 | 1.00 | 25.39 |
| 2317 | CD1 | PHE | B | 268 | 42.895 | −35.917 | 92.257 | 1.00 | 16.45 |
| 2318 | CD2 | PHE | B | 268 | 42.950 | −34.043 | 93.736 | 1.00 | 27.98 |
| 2319 | CE1 | PHE | B | 268 | 43.503 | −35.165 | 91.282 | 1.00 | 11.20 |
| 2320 | CE2 | PHE | B | 268 | 43.573 | −33.271 | 92.742 | 1.00 | 14.52 |
| 2321 | CZ | PHE | B | 268 | 43.848 | −33.837 | 91.523 | 1.00 | 14.38 |
| 2322 | C | PHE | B | 268 | 39.770 | −36.089 | 93.229 | 1.00 | 22.23 |
| 2323 | O | PHE | B | 268 | 39.548 | −35.130 | 92.488 | 1.00 | 24.28 |
| 2324 | N | ALA | B | 269 | 39.495 | −37.330 | 92.865 | 1.00 | 17.72 |
| 2325 | CA | ALA | B | 269 | 38.871 | −37.587 | 91.575 | 1.00 | 15.85 |
| 2326 | CB | ALA | B | 269 | 38.697 | −39.087 | 91.327 | 1.00 | 8.82 |
| 2327 | C | ALA | B | 269 | 37.527 | −36.913 | 91.601 | 1.00 | 14.49 |
| 2328 | O | ALA | B | 269 | 37.184 | −36.165 | 90.664 | 1.00 | 13.42 |
| 2329 | N | HIS | B | 270 | 36.778 | −37.160 | 92.684 | 1.00 | 12.69 |
| 2330 | CA | HIS | B | 270 | 35.453 | −36.570 | 92.828 | 1.00 | 17.33 |
| 2331 | CB | HIS | B | 270 | 34.978 | −36.787 | 94.254 | 1.00 | 8.12 |
| 2332 | CG | HIS | B | 270 | 33.552 | −36.393 | 94.493 | 1.00 | 10.92 |
| 2333 | CD2 | HIS | B | 270 | 33.006 | −35.389 | 95.224 | 1.00 | 15.72 |
| 2334 | ND1 | HIS | B | 270 | 32.491 | −37.145 | 94.040 | 1.00 | 5.25 |
| 2335 | CE1 | HIS | B | 270 | 31.359 | −36.637 | 94.491 | 1.00 | 5.25 |
| 2336 | NE2 | HIS | B | 270 | 31.643 | −35.572 | 95.216 | 1.00 | 5.25 |
| 2337 | C | HIS | B | 270 | 35.477 | −35.047 | 92.466 | 1.00 | 17.96 |
| 2338 | O | HIS | B | 270 | 34.770 | −34.568 | 91.570 | 1.00 | 18.90 |
| 2339 | N | PHE | B | 271 | 36.333 | −34.300 | 93.140 | 1.00 | 20.16 |
| 2340 | CA | PHE | B | 271 | 36.426 | −32.876 | 92.880 | 1.00 | 23.78 |
| 2341 | CB | PHE | B | 271 | 37.450 | −32.216 | 93.790 | 1.00 | 21.72 |
| 2342 | CG | PHE | B | 271 | 36.886 | −31.701 | 95.056 | 1.00 | 14.87 |
| 2343 | CD1 | PHE | B | 271 | 35.683 | −32.169 | 95.537 | 1.00 | 19.48 |
| 2344 | CD2 | PHE | B | 271 | 37.608 | −30.792 | 95.821 | 1.00 | 25.55 |
| 2345 | CE1 | PHE | B | 271 | 35.184 | −31.742 | 96.799 | 1.00 | 37.20 |
| 2346 | CE2 | PHE | B | 271 | 37.133 | −30.354 | 97.076 | 1.00 | 36.95 |
| 2347 | CZ | PHE | B | 271 | 35.906 | −30.839 | 97.570 | 1.00 | 35.14 |
| 2348 | C | PHE | B | 271 | 36.786 | −32.579 | 91.457 | 1.00 | 22.69 |
| 2349 | O | PHE | B | 271 | 36.042 | −31.890 | 90.800 | 1.00 | 21.96 |
| 2350 | N | THR | B | 272 | 37.917 | −33.102 | 90.989 | 1.00 | 25.49 |
| 2351 | CA | THR | B | 272 | 38.384 | −32.863 | 89.626 | 1.00 | 27.42 |
| 2352 | CB | THR | B | 272 | 39.608 | −33.695 | 89.306 | 1.00 | 26.81 |
| 2353 | OG1 | THR | B | 272 | 39.429 | −35.012 | 89.825 | 1.00 | 31.00 |
| 2354 | CG2 | THR | B | 272 | 40.847 | −33.075 | 89.948 | 1.00 | 35.42 |
| 2355 | C | THR | B | 272 | 37.309 | −33.174 | 88.612 | 1.00 | 32.00 |
| 2356 | O | THR | B | 272 | 37.042 | −32.340 | 87.722 | 1.00 | 35.46 |
| 2357 | N | GLU | B | 273 | 36.669 | −34.348 | 88.731 | 1.00 | 31.84 |
| 2358 | CA | GLU | B | 273 | 35.600 | −34.701 | 87.785 | 1.00 | 28.90 |
| 2359 | CB | GLU | B | 273 | 35.083 | −36.112 | 88.056 | 1.00 | 24.12 |
| 2360 | CG | GLU | B | 273 | 36.140 | −37.185 | 87.911 | 1.00 | 23.98 |
| 2361 | CD | GLU | B | 273 | 35.649 | −38.584 | 88.369 | 1.00 | 23.19 |
| 2362 | OE1 | GLU | B | 273 | 36.451 | −39.488 | 88.617 | 1.00 | 21.84 |
| 2363 | OE2 | GLU | B | 273 | 34.446 | −38.797 | 88.483 | 1.00 | 23.39 |
| 2364 | C | GLU | B | 273 | 34.434 | −33.684 | 87.779 | 1.00 | 26.54 |
| 2365 | O | GLU | B | 273 | 33.786 | −33.498 | 86.749 | 1.00 | 26.59 |
| 2366 | N | LEU | B | 274 | 34.177 | −33.031 | 88.916 | 1.00 | 23.99 |
| 2367 | CA | LEU | B | 274 | 33.110 | −32.034 | 89.008 | 1.00 | 24.74 |
| 2368 | CB | LEU | B | 274 | 32.719 | −31.761 | 90.483 | 1.00 | 26.78 |
| 2369 | CG | LEU | B | 274 | 31.960 | −32.918 | 91.164 | 1.00 | 32.47 |
| 2370 | CD1 | LEU | B | 274 | 31.737 | −32.573 | 92.582 | 1.00 | 35.05 |
| 2371 | CD2 | LEU | B | 274 | 30.627 | −33.199 | 90.490 | 1.00 | 12.62 |
| 2372 | C | LEU | B | 274 | 33.590 | −30.756 | 88.334 | 1.00 | 22.06 |
| 2373 | O | LEU | B | 274 | 32.895 | −30.191 | 87.499 | 1.00 | 23.23 |
| 2374 | N | ALA | B | 275 | 34.788 | −30.318 | 88.701 | 1.00 | 17.79 |
| 2375 | CA | ALA | B | 275 | 35.415 | −29.136 | 88.138 | 1.00 | 16.53 |
| 2376 | CB | ALA | B | 275 | 36.901 | −29.184 | 88.419 | 1.00 | 13.07 |
| 2377 | C | ALA | B | 275 | 35.173 | −29.077 | 86.633 | 1.00 | 18.81 |
| 2378 | O | ALA | B | 275 | 34.791 | −28.025 | 86.097 | 1.00 | 16.04 |
| 2379 | N | ILE | B | 276 | 35.410 | −30.213 | 85.965 | 1.00 | 16.66 |
| 2380 | CA | ILE | B | 276 | 35.211 | −30.336 | 84.532 | 1.00 | 16.32 |
| 2381 | CB | ILE | B | 276 | 35.528 | −31.759 | 84.053 | 1.00 | 13.21 |
| 2382 | CG2 | ILE | B | 276 | 35.144 | −31.904 | 82.626 | 1.00 | 5.25 |
| 2383 | CG1 | ILE | B | 276 | 37.027 | −32.038 | 84.263 | 1.00 | 15.91 |
| 2384 | CD1 | ILE | B | 276 | 37.448 | −33.430 | 83.945 | 1.00 | 13.43 |
| 2385 | C | ILE | B | 276 | 33.797 | −29.959 | 84.104 | 1.00 | 17.75 |
| 2386 | O | ILE | B | 276 | 33.616 | −29.173 | 83.162 | 1.00 | 18.39 |
| 2387 | N | ILE | B | 277 | 32.789 | −30.504 | 84.779 | 1.00 | 18.95 |
| 2388 | CA | ILE | B | 277 | 31.409 | −30.146 | 84.417 | 1.00 | 19.47 |
| 2389 | CB | ILE | B | 277 | 30.370 | −30.865 | 85.313 | 1.00 | 13.25 |
| 2390 | CG2 | ILE | B | 277 | 28.989 | −30.524 | 84.874 | 1.00 | 5.25 |
| 2391 | CG1 | ILE | B | 277 | 30.580 | −32.367 | 85.238 | 1.00 | 17.70 |
| 2392 | CD1 | ILE | B | 277 | 29.506 | −33.160 | 85.901 | 1.00 | 18.78 |
| 2393 | C | ILE | B | 277 | 31.311 | −28.627 | 84.646 | 1.00 | 20.85 |
| 2394 | O | ILE | B | 277 | 30.694 | −27.878 | 83.889 | 1.00 | 19.66 |
| 2395 | N | SER | B | 278 | 31.950 | −28.173 | 85.711 | 1.00 | 22.75 |
| 2396 | CA | SER | B | 278 | 31.931 | −26.768 | 86.016 | 1.00 | 23.72 |
| 2397 | CB | SER | B | 278 | 32.685 | −26.516 | 87.317 | 1.00 | 21.12 |
| 2398 | OG | SER | B | 278 | 32.715 | −25.120 | 87.581 | 1.00 | 22.54 |
| 2399 | C | SER | B | 278 | 32.536 | −25.924 | 84.853 | 1.00 | 25.47 |
| 2400 | O | SER | B | 278 | 31.984 | −24.852 | 84.531 | 1.00 | 25.59 |
| 2401 | N | VAL | B | 279 | 33.649 | −26.380 | 84.245 | 1.00 | 22.83 |
| 2402 | CA | VAL | B | 279 | 34.256 | −25.631 | 83.142 | 1.00 | 21.95 |
| 2403 | CB | VAL | B | 279 | 35.545 | −26.284 | 82.603 | 1.00 | 19.86 |
| 2404 | CG1 | VAL | B | 279 | 36.057 | −25.500 | 81.376 | 1.00 | 9.36 |
| 2405 | CG2 | VAL | B | 279 | 36.610 | −26.268 | 83.653 | 1.00 | 16.85 |
| 2406 | C | VAL | B | 279 | 33.263 | −25.553 | 81.992 | 1.00 | 23.30 |
| 2407 | O | VAL | B | 279 | 33.157 | −24.526 | 81.301 | 1.00 | 25.73 |
| 2408 | N | GLN | B | 280 | 32.525 | −26.640 | 81.803 | 1.00 | 21.66 |
| 2409 | CA | GLN | B | 280 | 31.527 | −26.697 | 80.766 | 1.00 | 20.75 |
| 2410 | CB | GLN | B | 280 | 30.966 | −28.120 | 80.653 | 1.00 | 25.14 |
| 2411 | CG | GLN | B | 280 | 32.012 | −29.144 | 80.267 | 1.00 | 22.03 |
| 2412 | CD | GLN | B | 280 | 31.458 | −30.563 | 80.305 | 1.00 | 41.57 |
| 2413 | OE1 | GLN | B | 280 | 30.340 | −30.809 | 80.804 | 1.00 | 47.16 |
| 2414 | NE2 | GLN | B | 280 | 32.236 | −31.509 | 79.783 | 1.00 | 36.98 |
| 2415 | C | GLN | B | 280 | 30.398 | −25.697 | 81.047 | 1.00 | 19.68 |
| 2416 | O | GLN | B | 280 | 29.809 | −25.156 | 80.093 | 1.00 | 17.78 |
| 2417 | N | GLU | B | 281 | 30.064 | −25.473 | 82.329 | 1.00 | 16.74 |
| 2418 | CA | GLU | B | 281 | 29.014 | −24.495 | 82.636 | 1.00 | 16.62 |
| 2419 | CB | GLU | B | 281 | 28.607 | −24.546 | 84.098 | 1.00 | 8.05 |
| 2420 | CG | GLU | B | 281 | 27.475 | −23.574 | 84.423 | 1.00 | 25.22 |
| 2421 | CD | GLU | B | 281 | 27.028 | −23.649 | 85.882 | 1.00 | 29.00 |
| 2422 | OE1 | GLU | B | 281 | 26.021 | −22.965 | 86.242 | 1.00 | 29.66 |
| 2423 | OE2 | GLU | B | 281 | 27.692 | −24.391 | 86.656 | 1.00 | 27.05 |
| 2424 | C | GLU | B | 281 | 29.545 | −23.084 | 82.280 | 1.00 | 16.32 |
| 2425 | O | GLU | B | 281 | 28.867 | −22.312 | 81.548 | 1.00 | 9.48 |
| 2426 | N | ILE | B | 282 | 30.755 | −22.779 | 82.786 | 1.00 | 15.22 |
| 2427 | CA | ILE | B | 282 | 31.429 | −21.515 | 82.531 | 1.00 | 16.61 |
| 2428 | CB | ILE | B | 282 | 32.798 | −21.492 | 83.185 | 1.00 | 12.62 |
| 2429 | CG2 | ILE | B | 282 | 33.667 | −20.365 | 82.596 | 1.00 | 11.77 |
| 2430 | CG1 | ILE | B | 282 | 32.587 | −21.243 | 84.673 | 1.00 | 15.38 |
| 2431 | CD1 | ILE | B | 282 | 33.841 | −21.159 | 85.506 | 1.00 | 8.99 |
| 2432 | C | ILE | B | 282 | 31.550 | −21.212 | 81.040 | 1.00 | 18.29 |
| 2433 | O | ILE | B | 282 | 31.031 | −20.180 | 80.558 | 1.00 | 14.48 |
| 2434 | N | VAL | B | 283 | 32.252 | −22.082 | 80.316 | 1.00 | 15.81 |
| 2435 | CA | VAL | B | 283 | 32.363 | −21.908 | 78.874 | 1.00 | 15.19 |
| 2436 | CB | VAL | B | 283 | 33.009 | −23.127 | 78.275 | 1.00 | 7.26 |
| 2437 | CG1 | VAL | B | 283 | 32.934 | −23.062 | 76.811 | 1.00 | 7.61 |
| 2438 | CG2 | VAL | B | 283 | 34.431 | −23.206 | 78.710 | 1.00 | 6.21 |
| 2439 | C | VAL | B | 283 | 30.951 | −21.706 | 78.221 | 1.00 | 17.61 |
| 2440 | O | VAL | B | 283 | 30.755 | −20.882 | 77.325 | 1.00 | 20.88 |
| 2441 | N | ASP | B | 284 | 29.966 | −22.435 | 78.721 | 1.00 | 18.45 |
| 2442 | CA | ASP | B | 284 | 28.614 | −22.388 | 78.210 | 1.00 | 21.25 |
| 2443 | CB | ASP | B | 284 | 27.825 | −23.516 | 78.871 | 1.00 | 35.01 |
| 2444 | CG | ASP | B | 284 | 26.585 | −23.902 | 78.114 | 1.00 | 40.84 |
| 2445 | OD1 | ASP | B | 284 | 26.695 | −24.069 | 76.884 | 1.00 | 58.98 |
| 2446 | OD2 | ASP | B | 284 | 25.515 | −24.074 | 78.749 | 1.00 | 47.00 |
| 2447 | C | ASP | B | 284 | 27.957 | −21.054 | 78.497 | 1.00 | 22.42 |
| 2448 | O | ASP | B | 284 | 27.123 | −20.628 | 77.745 | 1.00 | 25.11 |
| 2449 | N | PHE | B | 285 | 28.310 | −20.419 | 79.607 | 1.00 | 23.18 |
| 2450 | CA | PHE | B | 285 | 27.731 | −19.141 | 79.984 | 1.00 | 20.36 |
| 2451 | CB | PHE | B | 285 | 27.863 | −18.927 | 81.491 | 1.00 | 15.46 |
| 2452 | CG | PHE | B | 285 | 27.184 | −17.683 | 81.969 | 1.00 | 28.69 |
| 2453 | CD1 | PHE | B | 285 | 25.779 | −17.633 | 82.025 | 1.00 | 22.82 |
| 2454 | CD2 | PHE | B | 285 | 27.931 | −16.539 | 82.310 | 1.00 | 22.64 |
| 2455 | CE1 | PHE | B | 285 | 25.129 | −16.474 | 82.408 | 1.00 | 29.41 |
| 2456 | CE2 | PHE | B | 285 | 27.287 | −15.364 | 82.698 | 1.00 | 34.28 |
| 2457 | CZ | PHE | B | 285 | 25.883 | −15.322 | 82.749 | 1.00 | 33.57 |
| 2458 | C | PHE | B | 285 | 28.424 | −17.972 | 79.244 | 1.00 | 22.96 |
| 2459 | O | PHE | B | 285 | 27.771 | −16.987 | 78.851 | 1.00 | 26.62 |
| 2460 | N | ALA | B | 286 | 29.747 | −18.050 | 79.085 | 1.00 | 19.90 |
| 2461 | CA | ALA | B | 286 | 30.460 | −17.012 | 78.347 | 1.00 | 19.13 |
| 2462 | CB | ALA | B | 286 | 31.948 | −17.373 | 78.215 | 1.00 | 14.49 |
| 2463 | C | ALA | B | 286 | 29.794 | −16.865 | 76.949 | 1.00 | 18.53 |
| 2464 | O | ALA | B | 286 | 29.433 | −15.754 | 76.538 | 1.00 | 20.73 |
| 2465 | N | LYS | B | 287 | 29.625 | −17.982 | 76.240 | 1.00 | 15.74 |
| 2466 | CA | LYS | B | 287 | 28.995 | −17.958 | 74.942 | 1.00 | 21.37 |
| 2467 | CB | LYS | B | 287 | 28.667 | −19.373 | 74.480 | 1.00 | 33.02 |
| 2468 | CG | LYS | B | 287 | 29.817 | −20.189 | 73.913 | 1.00 | 30.82 |
| 2469 | CD | LYS | B | 287 | 29.255 | −21.152 | 72.890 | 1.00 | 40.12 |
| 2470 | CE | LYS | B | 287 | 30.245 | −22.209 | 72.466 | 1.00 | 44.68 |
| 2471 | NZ | LYS | B | 287 | 30.510 | −23.148 | 73.581 | 1.00 | 37.69 |
| 2472 | C | LYS | B | 287 | 27.702 | −17.157 | 74.953 | 1.00 | 22.71 |
| 2473 | O | LYS | B | 287 | 27.255 | −16.707 | 73.910 | 1.00 | 22.47 |
| 2474 | N | GLN | B | 288 | 27.117 | −16.967 | 76.138 | 1.00 | 25.38 |
| 2475 | CA | GLN | B | 288 | 25.841 | −16.256 | 76.278 | 1.00 | 23.59 |
| 2476 | CB | GLN | B | 288 | 24.912 | −17.076 | 77.176 | 1.00 | 24.27 |
| 2477 | CG | GLN | B | 288 | 24.675 | −18.469 | 76.639 | 1.00 | 28.01 |
| 2478 | CD | GLN | B | 288 | 23.224 | −18.774 | 76.525 | 1.00 | 42.38 |
| 2479 | OE1 | GLN | B | 288 | 22.640 | −19.488 | 77.363 | 1.00 | 51.37 |
| 2480 | NE2 | GLN | B | 288 | 22.601 | −18.212 | 75.498 | 1.00 | 45.89 |
| 2481 | C | GLN | B | 288 | 25.919 | −14.817 | 76.777 | 1.00 | 20.41 |
| 2482 | O | GLN | B | 288 | 24.895 | −14.098 | 76.809 | 1.00 | 18.32 |
| 2483 | N | VAL | B | 289 | 27.122 | −14.396 | 77.160 | 1.00 | 17.16 |
| 2484 | CA | VAL | B | 289 | 27.303 | −13.035 | 77.633 | 1.00 | 18.12 |
| 2485 | CB | VAL | B | 289 | 28.555 | −12.911 | 78.503 | 1.00 | 18.92 |
| 2486 | CG1 | VAL | B | 289 | 28.756 | −11.468 | 78.863 | 1.00 | 7.11 |
| 2487 | CG2 | VAL | B | 289 | 28.405 | −13.791 | 79.757 | 1.00 | 19.44 |
| 2488 | C | VAL | B | 289 | 27.472 | −12.161 | 76.415 | 1.00 | 19.12 |
| 2489 | O | VAL | B | 289 | 28.429 | −12.304 | 75.657 | 1.00 | 22.10 |
| 2490 | N | PRO | B | 290 | 26.540 | −11.250 | 76.189 | 1.00 | 17.24 |
| 2491 | CD | PRO | B | 290 | 25.445 | −10.859 | 77.086 | 1.00 | 19.88 |
| 2492 | CA | PRO | B | 290 | 26.624 | −10.359 | 75.022 | 1.00 | 16.50 |
| 2493 | CB | PRO | B | 290 | 25.610 | −9.268 | 75.345 | 1.00 | 9.12 |
| 2494 | CG | PRO | B | 290 | 24.614 | −9.963 | 76.177 | 1.00 | 15.86 |
| 2495 | C | PRO | B | 290 | 28.031 | −9.776 | 74.811 | 1.00 | 20.18 |
| 2496 | O | PRO | B | 290 | 28.652 | −9.221 | 75.734 | 1.00 | 19.50 |
| 2497 | N | GLY | B | 291 | 28.533 | −9.899 | 73.589 | 1.00 | 24.22 |
| 2498 | CA | GLY | B | 291 | 29.840 | −9.354 | 73.308 | 1.00 | 19.13 |
| 2499 | C | GLY | B | 291 | 30.901 | −10.410 | 73.325 | 1.00 | 18.84 |
| 2500 | O | GLY | B | 291 | 31.825 | −10.372 | 72.525 | 1.00 | 25.00 |
| 2501 | N | PHE | B | 292 | 30.768 | −11.383 | 74.206 | 1.00 | 18.68 |
| 2502 | CA | PHE | B | 292 | 31.795 | −12.397 | 74.250 | 1.00 | 19.53 |
| 2503 | CB | PHE | B | 292 | 31.475 | −13.491 | 75.280 | 1.00 | 19.98 |
| 2504 | CG | PHE | B | 292 | 32.624 | −14.467 | 75.489 | 1.00 | 16.63 |
| 2505 | CD1 | PHE | B | 292 | 33.768 | −14.058 | 76.131 | 1.00 | 7.41 |
| 2506 | CD2 | PHE | B | 292 | 32.565 | −15.776 | 75.005 | 1.00 | 19.33 |
| 2507 | CE1 | PHE | B | 292 | 34.838 | −14.916 | 76.296 | 1.00 | 14.65 |
| 2508 | CE2 | PHE | B | 292 | 33.635 | −16.651 | 75.167 | 1.00 | 16.74 |
| 2509 | CZ | PHE | B | 292 | 34.778 | −16.227 | 75.817 | 1.00 | 15.51 |
| 2510 | C | PHE | B | 292 | 32.113 | −13.051 | 72.913 | 1.00 | 18.23 |
| 2511 | O | PHE | B | 292 | 33.291 | −13.184 | 72.576 | 1.00 | 19.50 |
| 2512 | N | LEU | B | 293 | 31.089 | −13.479 | 72.162 | 1.00 | 18.86 |
| 2513 | CA | LEU | B | 293 | 31.334 | −14.145 | 70.851 | 1.00 | 21.39 |
| 2514 | CB | LEU | B | 293 | 30.117 | −14.989 | 70.407 | 1.00 | 12.01 |
| 2515 | CG | LEU | B | 293 | 29.913 | −16.208 | 71.350 | 1.00 | 20.64 |
| 2516 | CD1 | LEU | B | 293 | 28.598 | −16.911 | 71.160 | 1.00 | 14.53 |
| 2517 | CD2 | LEU | B | 293 | 31.060 | −17.156 | 71.128 | 1.00 | 13.13 |
| 2518 | C | LEU | B | 293 | 31.762 | −13.226 | 69.701 | 1.00 | 22.36 |
| 2519 | O | LEU | B | 293 | 31.917 | −13.661 | 68.571 | 1.00 | 22.27 |
| 2520 | N | GLN | B | 294 | 31.965 | −11.954 | 70.000 | 1.00 | 24.19 |
| 2521 | CA | GLN | B | 294 | 32.425 | −11.047 | 68.985 | 1.00 | 27.26 |
| 2522 | CB | GLN | B | 294 | 31.917 | −9.663 | 69.284 | 1.00 | 30.86 |
| 2523 | CG | GLN | B | 294 | 30.408 | −9.595 | 69.201 | 1.00 | 42.01 |
| 2524 | CD | GLN | B | 294 | 29.894 | −8.225 | 69.558 | 1.00 | 49.66 |
| 2525 | OE1 | GLN | B | 294 | 28.697 | −7.945 | 69.428 | 1.00 | 57.97 |
| 2526 | NE2 | GLN | B | 294 | 30.800 | −7.353 | 70.019 | 1.00 | 49.92 |
| 2527 | C | GLN | B | 294 | 33.955 | −11.067 | 68.945 | 1.00 | 27.72 |
| 2528 | O | GLN | B | 294 | 34.558 | −10.640 | 67.967 | 1.00 | 31.18 |
| 2529 | N | LEU | B | 295 | 34.586 | −11.568 | 70.005 | 1.00 | 23.34 |
| 2530 | CA | LEU | B | 295 | 36.038 | −11.637 | 70.045 | 1.00 | 19.78 |
| 2531 | CB | LEU | B | 295 | 36.579 | −11.666 | 71.494 | 1.00 | 13.80 |
| 2532 | CG | LEU | B | 295 | 36.213 | −10.437 | 72.368 | 1.00 | 20.64 |
| 2533 | CD1 | LEU | B | 295 | 36.591 | −10.633 | 73.831 | 1.00 | 16.87 |
| 2534 | CD2 | LEU | B | 295 | 36.833 | −9.207 | 71.780 | 1.00 | 5.37 |
| 2535 | C | LEU | B | 295 | 36.522 | −12.861 | 69.286 | 1.00 | 23.15 |
| 2536 | O | LEU | B | 295 | 35.770 | −13.808 | 69.013 | 1.00 | 27.08 |
| 2537 | N | GLY | B | 296 | 37.797 | −12.829 | 68.930 | 1.00 | 20.51 |
| 2538 | CA | GLY | B | 296 | 38.369 | −13.926 | 68.181 | 1.00 | 22.00 |
| 2539 | C | GLY | B | 296 | 38.613 | −15.083 | 69.115 | 1.00 | 24.52 |
| 2540 | O | GLY | B | 296 | 38.856 | −14.879 | 70.294 | 1.00 | 26.91 |
| 2541 | N | ARG | B | 297 | 38.544 | −16.299 | 68.585 | 1.00 | 24.81 |
| 2542 | CA | ARG | B | 297 | 38.758 | −17.494 | 69.369 | 1.00 | 22.38 |
| 2543 | CB | ARG | B | 297 | 38.884 | −18.685 | 68.435 | 1.00 | 14.04 |
| 2544 | CG | ARG | B | 297 | 37.824 | −19.715 | 68.690 | 1.00 | 27.39 |
| 2545 | CD | ARG | B | 297 | 36.525 | −19.321 | 68.018 | 1.00 | 38.77 |
| 2546 | NE | ARG | B | 297 | 35.344 | −19.780 | 68.741 | 1.00 | 36.12 |
| 2547 | CZ | ARG | B | 297 | 34.228 | −20.166 | 68.148 | 1.00 | 41.67 |
| 2548 | NH1 | ARG | B | 297 | 34.151 | −20.165 | 66.833 | 1.00 | 52.02 |
| 2549 | NH2 | ARG | B | 297 | 33.177 | −20.513 | 68.876 | 1.00 | 59.39 |
| 2550 | C | ARG | B | 297 | 39.983 | −17.389 | 70.315 | 1.00 | 22.84 |
| 2551 | O | ARG | B | 297 | 39.952 | −17.875 | 71.470 | 1.00 | 24.28 |
| 2552 | N | GLU | B | 298 | 41.047 | −16.752 | 69.835 | 1.00 | 20.17 |
| 2553 | CA | GLU | B | 298 | 42.249 | −16.567 | 70.636 | 1.00 | 18.15 |
| 2554 | CB | GLU | B | 298 | 43.283 | −15.806 | 69.799 | 1.00 | 24.82 |
| 2555 | CG | GLU | B | 298 | 43.994 | −16.663 | 68.776 | 1.00 | 40.30 |
| 2556 | CD | GLU | B | 298 | 44.975 | −17.637 | 69.425 | 1.00 | 59.47 |
| 2557 | OE1 | GLU | B | 298 | 45.919 | −17.155 | 70.101 | 1.00 | 73.88 |
| 2558 | OE2 | GLU | B | 298 | 44.809 | −18.879 | 69.280 | 1.00 | 61.89 |
| 2559 | C | GLU | B | 298 | 41.944 | −15.827 | 71.965 | 1.00 | 17.48 |
| 2560 | O | GLU | B | 298 | 42.270 | −16.311 | 73.054 | 1.00 | 15.91 |
| 2561 | N | ASP | B | 299 | 41.301 | −14.664 | 71.865 | 1.00 | 19.09 |
| 2562 | CA | ASP | B | 299 | 40.951 | −13.852 | 73.018 | 1.00 | 16.81 |
| 2563 | CB | ASP | B | 299 | 40.294 | −12.534 | 72.585 | 1.00 | 21.76 |
| 2564 | CG | ASP | B | 299 | 41.279 | −11.505 | 72.019 | 1.00 | 28.31 |
| 2565 | OD1 | ASP | B | 299 | 42.310 | −11.253 | 72.659 | 1.00 | 26.29 |
| 2566 | OD2 | ASP | B | 299 | 41.011 | −10.919 | 70.930 | 1.00 | 42.01 |
| 2567 | C | ASP | B | 299 | 39.949 | −14.587 | 73.902 | 1.00 | 18.84 |
| 2568 | O | ASP | B | 299 | 39.937 | −14.347 | 75.117 | 1.00 | 19.02 |
| 2569 | N | GLN | B | 300 | 39.107 | −15.444 | 73.293 | 1.00 | 14.38 |
| 2570 | CA | GLN | B | 300 | 38.072 | −16.181 | 74.015 | 1.00 | 14.01 |
| 2571 | CB | GLN | B | 300 | 37.160 | −16.932 | 73.057 | 1.00 | 12.01 |
| 2572 | CG | GLN | B | 300 | 35.794 | −16.406 | 72.996 | 1.00 | 19.29 |
| 2573 | CD | GLN | B | 300 | 35.069 | −16.819 | 71.734 | 1.00 | 35.24 |
| 2574 | OE1 | GLN | B | 300 | 35.417 | −16.386 | 70.634 | 1.00 | 32.75 |
| 2575 | NE2 | GLN | B | 300 | 34.051 | −17.660 | 71.882 | 1.00 | 41.69 |
| 2576 | C | GLN | B | 300 | 38.695 | −17.166 | 74.927 | 1.00 | 14.12 |
| 2577 | O | GLN | B | 300 | 38.340 | −17.265 | 76.094 | 1.00 | 15.64 |
| 2578 | N | ILE | B | 301 | 39.629 | −17.918 | 74.374 | 1.00 | 15.96 |
| 2579 | CA | ILE | B | 301 | 40.310 | −18.921 | 75.151 | 1.00 | 17.05 |
| 2580 | CB | ILE | B | 301 | 41.218 | −19.750 | 74.249 | 1.00 | 11.32 |
| 2581 | CG2 | ILE | B | 301 | 42.140 | −20.674 | 75.096 | 1.00 | 5.57 |
| 2582 | CG1 | ILE | B | 301 | 40.307 | −20.560 | 73.313 | 1.00 | 10.47 |
| 2583 | CD1 | ILE | B | 301 | 40.956 | −20.976 | 72.023 | 1.00 | 8.58 |
| 2584 | C | ILE | B | 301 | 41.073 | −18.178 | 76.204 | 1.00 | 16.30 |
| 2585 | O | ILE | B | 301 | 40.911 | −18.422 | 77.382 | 1.00 | 14.82 |
| 2586 | N | ALA | B | 302 | 41.904 | −17.247 | 75.781 | 1.00 | 18.66 |
| 2587 | CA | ALA | B | 302 | 42.660 | −16.459 | 76.764 | 1.00 | 22.11 |
| 2588 | CB | ALA | B | 302 | 43.287 | −15.206 | 76.078 | 1.00 | 25.04 |
| 2589 | C | ALA | B | 302 | 41.803 | −16.005 | 77.985 | 1.00 | 20.32 |
| 2590 | O | ALA | B | 302 | 42.133 | −16.284 | 79.148 | 1.00 | 17.57 |
| 2591 | N | LEU | B | 303 | 40.713 | −15.295 | 77.700 | 1.00 | 17.46 |
| 2592 | CA | LEU | B | 303 | 39.823 | −14.803 | 78.741 | 1.00 | 18.81 |
| 2593 | CB | LEU | B | 303 | 38.724 | −13.935 | 78.154 | 1.00 | 7.57 |
| 2594 | CG | LEU | B | 303 | 39.180 | −12.585 | 77.629 | 1.00 | 7.97 |
| 2595 | CD1 | LEU | B | 303 | 37.974 | −11.752 | 77.334 | 1.00 | 15.12 |
| 2596 | CD2 | LEU | B | 303 | 40.013 | −11.885 | 78.686 | 1.00 | 15.05 |
| 2597 | C | LEU | B | 303 | 39.181 | −15.880 | 79.613 | 1.00 | 21.50 |
| 2598 | O | LEU | B | 303 | 38.929 | −15.651 | 80.792 | 1.00 | 23.19 |
| 2599 | N | LEU | B | 304 | 38.901 | −17.046 | 79.038 | 1.00 | 21.15 |
| 2600 | CA | LEU | B | 304 | 38.330 | −18.127 | 79.819 | 1.00 | 17.97 |
| 2601 | CB | LEU | B | 304 | 37.754 | −19.181 | 78.925 | 1.00 | 16.23 |
| 2602 | CG | LEU | B | 304 | 36.346 | −18.908 | 78.446 | 1.00 | 19.16 |
| 2603 | CD1 | LEU | B | 304 | 35.689 | −20.216 | 78.103 | 1.00 | 34.29 |
| 2604 | CD2 | LEU | B | 304 | 35.582 | −18.246 | 79.514 | 1.00 | 25.48 |
| 2605 | C | LEU | B | 304 | 39.405 | −18.760 | 80.676 | 1.00 | 19.88 |
| 2606 | O | LEU | B | 304 | 39.251 | −18.916 | 81.893 | 1.00 | 26.25 |
| 2607 | N | LYS | B | 305 | 40.499 | −19.121 | 80.029 | 1.00 | 14.19 |
| 2608 | CA | LYS | B | 305 | 41.629 | −19.721 | 80.708 | 1.00 | 17.19 |
| 2609 | CB | LYS | B | 305 | 42.858 | −19.681 | 79.767 | 1.00 | 19.18 |
| 2610 | CG | LYS | B | 305 | 44.134 | −20.300 | 80.336 | 1.00 | 22.69 |
| 2611 | CD | LYS | B | 305 | 45.188 | −20.446 | 79.256 | 1.00 | 31.13 |
| 2612 | CE | LYS | B | 305 | 46.538 | −20.816 | 79.794 | 1.00 | 42.37 |
| 2613 | NZ | LYS | B | 305 | 46.514 | −22.177 | 80.375 | 1.00 | 54.77 |
| 2614 | C | LYS | B | 305 | 41.959 | −19.070 | 82.070 | 1.00 | 15.90 |
| 2615 | O | LYS | B | 305 | 42.185 | −19.735 | 83.047 | 1.00 | 19.41 |
| 2616 | N | ALA | B | 306 | 41.958 | −17.760 | 82.145 | 1.00 | 18.52 |
| 2617 | CA | ALA | B | 306 | 42.283 | −17.111 | 83.389 | 1.00 | 15.98 |
| 2618 | CB | ALA | B | 306 | 42.919 | −15.776 | 83.123 | 1.00 | 11.91 |
| 2619 | C | ALA | B | 306 | 41.117 | −16.914 | 84.322 | 1.00 | 17.98 |
| 2620 | O | ALA | B | 306 | 41.329 | −16.974 | 85.537 | 1.00 | 19.87 |
| 2621 | N | SER | B | 307 | 39.905 | −16.680 | 83.792 | 1.00 | 17.73 |
| 2622 | CA | SER | B | 307 | 38.739 | −16.421 | 84.672 | 1.00 | 25.04 |
| 2623 | CB | SER | B | 307 | 37.692 | −15.593 | 83.953 | 1.00 | 24.67 |
| 2624 | OG | SER | B | 307 | 37.262 | −16.308 | 82.812 | 1.00 | 33.34 |
| 2625 | C | SER | B | 307 | 38.031 | −17.634 | 85.282 | 1.00 | 25.83 |
| 2626 | O | SER | B | 307 | 37.280 | −17.475 | 86.244 | 1.00 | 21.14 |
| 2627 | N | THR | B | 308 | 38.290 | −18.819 | 84.710 | 1.00 | 25.98 |
| 2628 | CA | THR | B | 308 | 37.691 | −20.064 | 85.149 | 1.00 | 18.07 |
| 2629 | CB | THR | B | 308 | 38.323 | −21.269 | 84.467 | 1.00 | 18.27 |
| 2630 | OG1 | THR | B | 308 | 38.066 | −21.207 | 83.066 | 1.00 | 12.82 |
| 2631 | CG2 | THR | B | 308 | 37.743 | −22.537 | 85.004 | 1.00 | 15.40 |
| 2632 | C | THR | B | 308 | 37.818 | −20.251 | 86.624 | 1.00 | 17.49 |
| 2633 | O | THR | B | 308 | 36.823 | −20.354 | 87.332 | 1.00 | 17.50 |
| 2634 | N | ILE | B | 309 | 39.040 | −20.259 | 87.111 | 1.00 | 15.34 |
| 2635 | CA | ILE | B | 309 | 39.235 | −20.467 | 88.539 | 1.00 | 15.21 |
| 2636 | CB | ILE | B | 309 | 40.738 | −20.482 | 88.886 | 1.00 | 13.09 |
| 2637 | CG2 | ILE | B | 309 | 41.349 | −19.105 | 88.725 | 1.00 | 20.27 |
| 2638 | CG1 | ILE | B | 309 | 40.926 | −20.891 | 90.312 | 1.00 | 10.55 |
| 2639 | CD1 | ILE | B | 309 | 40.405 | −22.213 | 90.598 | 1.00 | 10.98 |
| 2640 | C | ILE | B | 309 | 38.543 | −19.382 | 89.377 | 1.00 | 19.61 |
| 2641 | O | ILE | B | 309 | 37.942 | −19.662 | 90.437 | 1.00 | 17.36 |
| 2642 | N | GLU | B | 310 | 38.614 | −18.142 | 88.891 | 1.00 | 21.83 |
| 2643 | CA | GLU | B | 310 | 38.024 | −17.041 | 89.626 | 1.00 | 24.74 |
| 2644 | CB | GLU | B | 310 | 38.473 | −15.713 | 89.017 | 1.00 | 20.41 |
| 2645 | CG | GLU | B | 310 | 39.978 | −15.592 | 88.940 | 1.00 | 20.71 |
| 2646 | CD | GLU | B | 310 | 40.446 | −14.170 | 88.695 | 1.00 | 21.40 |
| 2647 | OE1 | GLU | B | 310 | 39.575 | −13.329 | 88.420 | 1.00 | 21.25 |
| 2648 | OE2 | GLU | B | 310 | 41.672 | −13.889 | 88.776 | 1.00 | 15.00 |
| 2649 | C | GLU | B | 310 | 36.491 | −17.137 | 89.712 | 1.00 | 25.09 |
| 2650 | O | GLU | B | 310 | 35.862 | −16.889 | 90.772 | 1.00 | 27.49 |
| 2651 | N | ILE | B | 311 | 35.890 | −17.510 | 88.595 | 1.00 | 23.06 |
| 2652 | CA | ILE | B | 311 | 34.455 | −17.680 | 88.538 | 1.00 | 20.02 |
| 2653 | CB | ILE | B | 311 | 33.986 | −18.013 | 87.077 | 1.00 | 22.03 |
| 2654 | CG2 | ILE | B | 311 | 32.467 | −18.325 | 87.037 | 1.00 | 24.02 |
| 2655 | CG1 | ILE | B | 311 | 34.283 | −16.829 | 86.154 | 1.00 | 13.12 |
| 2656 | CD1 | ILE | B | 311 | 33.588 | −16.899 | 84.849 | 1.00 | 5.25 |
| 2657 | C | ILE | B | 311 | 34.060 | −18.830 | 89.497 | 1.00 | 21.66 |
| 2658 | O | ILE | B | 311 | 33.125 | −18.690 | 90.303 | 1.00 | 19.46 |
| 2659 | N | MET | B | 312 | 34.781 | −19.954 | 89.436 | 1.00 | 16.11 |
| 2660 | CA | MET | B | 312 | 34.459 | −21.083 | 90.324 | 1.00 | 13.84 |
| 2661 | CB | MET | B | 312 | 35.479 | −22.210 | 90.188 | 1.00 | 14.56 |
| 2662 | CG | MET | B | 312 | 35.628 | −22.790 | 88.815 | 1.00 | 17.34 |
| 2663 | SD | MET | B | 312 | 36.931 | −24.091 | 88.709 | 1.00 | 16.56 |
| 2664 | CE | MET | B | 312 | 35.811 | −25.492 | 88.752 | 1.00 | 37.17 |
| 2665 | C | MET | B | 312 | 34.445 | −20.609 | 91.771 | 1.00 | 14.68 |
| 2666 | O | MET | B | 312 | 33.574 | −20.995 | 92.574 | 1.00 | 14.77 |
| 2667 | N | LEU | B | 313 | 35.423 | −19.782 | 92.112 | 1.00 | 12.44 |
| 2668 | CA | LEU | B | 313 | 35.449 | −19.255 | 93.465 | 1.00 | 15.01 |
| 2669 | CB | LEU | B | 313 | 36.771 | −18.557 | 93.683 | 1.00 | 11.43 |
| 2670 | CG | LEU | B | 313 | 37.924 | −19.537 | 93.730 | 1.00 | 13.95 |
| 2671 | CD1 | LEU | B | 313 | 39.176 | −18.752 | 93.734 | 1.00 | 24.08 |
| 2672 | CD2 | LEU | B | 313 | 37.846 | −20.418 | 94.976 | 1.00 | 5.25 |
| 2673 | C | LEU | B | 313 | 34.248 | −18.304 | 93.730 | 1.00 | 11.72 |
| 2674 | O | LEU | B | 313 | 33.721 | −18.215 | 94.869 | 1.00 | 5.25 |
| 2675 | N | LEU | B | 314 | 33.823 | −17.588 | 92.684 | 1.00 | 9.59 |
| 2676 | CA | LEU | B | 314 | 32.676 | −16.721 | 92.878 | 1.00 | 12.83 |
| 2677 | CB | LEU | B | 314 | 32.414 | −15.842 | 91.644 | 1.00 | 7.74 |
| 2678 | CG | LEU | B | 314 | 33.346 | −14.608 | 91.510 | 1.00 | 13.68 |
| 2679 | CD1 | LEU | B | 314 | 33.382 | −14.060 | 90.117 | 1.00 | 5.25 |
| 2680 | CD2 | LEU | B | 314 | 32.889 | −13.530 | 92.466 | 1.00 | 5.25 |
| 2681 | C | LEU | B | 314 | 31.491 | −17.625 | 93.200 | 1.00 | 12.72 |
| 2682 | O | LEU | B | 314 | 30.765 | −17.361 | 94.157 | 1.00 | 10.28 |
| 2683 | N | GLU | B | 315 | 31.332 | −18.716 | 92.437 | 1.00 | 16.60 |
| 2684 | CA | GLU | B | 315 | 30.235 | −19.676 | 92.655 | 1.00 | 19.32 |
| 2685 | CB | GLU | B | 315 | 30.249 | −20.797 | 91.623 | 1.00 | 20.97 |
| 2686 | CG | GLU | B | 315 | 29.891 | −20.418 | 90.256 | 1.00 | 25.79 |
| 2687 | CD | GLU | B | 315 | 28.474 | −20.048 | 90.136 | 1.00 | 24.04 |
| 2688 | OE1 | GLU | B | 315 | 27.633 | −20.671 | 90.812 | 1.00 | 30.51 |
| 2689 | OE2 | GLU | B | 315 | 28.192 | −19.129 | 89.350 | 1.00 | 35.73 |
| 2690 | C | GLU | B | 315 | 30.401 | −20.325 | 94.042 | 1.00 | 22.91 |
| 2691 | O | GLU | B | 315 | 29.436 | −20.433 | 94.829 | 1.00 | 25.74 |
| 2692 | N | THR | B | 316 | 31.620 | −20.783 | 94.330 | 1.00 | 19.17 |
| 2693 | CA | THR | B | 316 | 31.918 | −21.380 | 95.632 | 1.00 | 16.62 |
| 2694 | CB | THR | B | 316 | 33.393 | −21.538 | 95.830 | 1.00 | 17.06 |
| 2695 | OG1 | THR | B | 316 | 33.863 | −22.615 | 95.011 | 1.00 | 20.32 |
| 2696 | CG2 | THR | B | 316 | 33.669 | −21.801 | 97.232 | 1.00 | 14.20 |
| 2697 | C | THR | B | 316 | 31.418 | −20.448 | 96.704 | 1.00 | 13.14 |
| 2698 | O | THR | B | 316 | 30.828 | −20.897 | 97.644 | 1.00 | 17.68 |
| 2699 | N | ALA | B | 317 | 31.663 | −19.147 | 96.552 | 1.00 | 12.23 |
| 2700 | CA | ALA | B | 317 | 31.184 | −18.156 | 97.505 | 1.00 | 10.33 |
| 2701 | CB | ALA | B | 317 | 31.714 | −16.784 | 97.158 | 1.00 | 5.89 |
| 2702 | C | ALA | B | 317 | 29.658 | −18.134 | 97.491 | 1.00 | 13.35 |
| 2703 | O | ALA | B | 317 | 29.024 | −18.231 | 98.537 | 1.00 | 15.23 |
| 2704 | N | ARG | B | 318 | 29.063 | −18.003 | 96.310 | 1.00 | 14.74 |
| 2705 | CA | ARG | B | 318 | 27.610 | −17.976 | 96.210 | 1.00 | 14.84 |
| 2706 | CB | ARG | B | 318 | 27.193 | −17.799 | 94.741 | 1.00 | 14.50 |
| 2707 | CG | ARG | B | 318 | 25.694 | −18.024 | 94.503 | 1.00 | 14.19 |
| 2708 | CD | ARG | B | 318 | 25.184 | −17.373 | 93.220 | 1.00 | 25.75 |
| 2709 | NE | ARG | B | 318 | 25.755 | −17.926 | 91.994 | 1.00 | 23.63 |
| 2710 | CZ | ARG | B | 318 | 25.484 | −17.446 | 90.778 | 1.00 | 34.61 |
| 2711 | NH1 | ARG | B | 318 | 24.653 | −16.424 | 90.636 | 1.00 | 34.00 |
| 2712 | NH2 | ARG | B | 318 | 26.059 | −17.967 | 89.699 | 1.00 | 31.00 |
| 2713 | C | ARG | B | 318 | 26.892 | −19.202 | 96.808 | 1.00 | 17.70 |
| 2714 | O | ARG | B | 318 | 25.785 | −19.072 | 97.320 | 1.00 | 18.96 |
| 2715 | N | ARG | B | 319 | 27.509 | −20.386 | 96.726 | 1.00 | 20.72 |
| 2716 | CA | ARG | B | 319 | 26.921 | −21.632 | 97.244 | 1.00 | 16.92 |
| 2717 | CB | ARG | B | 319 | 27.303 | −22.800 | 96.329 | 1.00 | 18.39 |
| 2718 | CG | ARG | B | 319 | 26.921 | −22.642 | 94.898 | 1.00 | 5.97 |
| 2719 | CD | ARG | B | 319 | 25.751 | −23.607 | 94.553 | 1.00 | 28.69 |
| 2720 | NE | ARG | B | 319 | 25.163 | −23.287 | 93.253 | 1.00 | 11.54 |
| 2721 | CZ | ARG | B | 319 | 25.913 | −22.910 | 92.240 | 1.00 | 24.11 |
| 2722 | NH1 | ARG | B | 319 | 27.250 | −22.851 | 92.434 | 1.00 | 16.61 |
| 2723 | NH2 | ARG | B | 319 | 25.327 | −22.539 | 91.098 | 1.00 | 21.88 |
| 2724 | C | ARG | B | 319 | 27.390 | −21.940 | 98.677 | 1.00 | 13.41 |
| 2725 | O | ARG | B | 319 | 27.282 | −23.054 | 99.145 | 1.00 | 14.44 |
| 2726 | N | TYR | B | 320 | 27.919 | −20.938 | 99.359 | 1.00 | 16.42 |
| 2727 | CA | TYR | B | 320 | 28.386 | −21.074 | 100.733 | 1.00 | 19.36 |
| 2728 | CB | TYR | B | 320 | 29.586 | −20.170 | 100.965 | 1.00 | 17.23 |
| 2729 | CG | TYR | B | 320 | 29.990 | −20.112 | 102.426 | 1.00 | 12.95 |
| 2730 | CD1 | TYR | B | 320 | 29.587 | −19.082 | 103.223 | 1.00 | 9.66 |
| 2731 | CE1 | TYR | B | 320 | 29.974 | −18.993 | 104.518 | 1.00 | 7.34 |
| 2732 | CD2 | TYR | B | 320 | 30.802 | −21.073 | 102.983 | 1.00 | 19.08 |
| 2733 | CE2 | TYR | B | 320 | 31.200 | −21.001 | 104.292 | 1.00 | 14.64 |
| 2734 | CZ | TYR | B | 320 | 30.785 | −19.946 | 105.061 | 1.00 | 20.88 |
| 2735 | OH | TYR | B | 320 | 31.214 | −19.801 | 106.379 | 1.00 | 32.48 |
| 2736 | C | TYR | B | 320 | 27.318 | −20.724 | 101.766 | 1.00 | 22.75 |
| 2737 | O | TYR | B | 320 | 26.672 | −19.655 | 101.670 | 1.00 | 27.48 |
| 2738 | N | ASN | B | 321 | 27.122 | −21.591 | 102.759 | 1.00 | 23.31 |
| 2739 | CA | ASN | B | 321 | 26.109 | −21.283 | 103.782 | 1.00 | 29.74 |
| 2740 | CB | ASN | B | 321 | 25.036 | −22.398 | 103.883 | 1.00 | 33.20 |
| 2741 | CG | ASN | B | 321 | 25.077 | −23.161 | 105.188 | 1.00 | 28.54 |
| 2742 | OD1 | ASN | B | 321 | 24.968 | −22.604 | 106.274 | 1.00 | 21.25 |
| 2743 | ND2 | ASN | B | 321 | 25.216 | −24.462 | 105.075 | 1.00 | 38.75 |
| 2744 | C | ASN | B | 321 | 26.817 | −21.077 | 105.092 | 1.00 | 31.37 |
| 2745 | O | ASN | B | 321 | 27.676 | −21.870 | 105.472 | 1.00 | 29.68 |
| 2746 | N | HIS | B | 322 | 26.451 | −19.999 | 105.772 | 1.00 | 34.77 |
| 2747 | CA | HIS | B | 322 | 27.070 | −19.642 | 107.033 | 1.00 | 40.14 |
| 2748 | CB | HIS | B | 322 | 26.755 | −18.189 | 107.364 | 1.00 | 51.27 |
| 2749 | CG | HIS | B | 322 | 27.574 | −17.637 | 108.495 | 1.00 | 68.36 |
| 2750 | CD2 | HIS | B | 322 | 28.820 | −17.098 | 108.517 | 1.00 | 75.91 |
| 2751 | ND1 | HIS | B | 322 | 27.127 | −17.608 | 109.800 | 1.00 | 75.30 |
| 2752 | CE1 | HIS | B | 322 | 28.056 | −17.075 | 110.576 | 1.00 | 77.35 |
| 2753 | NE2 | HIS | B | 322 | 29.095 | −16.757 | 109.821 | 1.00 | 81.24 |
| 2754 | C | HIS | B | 322 | 26.741 | −20.512 | 108.234 | 1.00 | 41.26 |
| 2755 | O | HIS | B | 322 | 27.593 | −20.722 | 109.094 | 1.00 | 42.57 |
| 2756 | N | GLU | B | 323 | 25.530 | −21.040 | 108.310 | 1.00 | 42.04 |
| 2757 | CA | GLU | B | 323 | 25.206 | −21.849 | 109.469 | 1.00 | 45.58 |
| 2758 | CB | GLU | B | 323 | 23.682 | −22.076 | 109.546 | 1.00 | 44.87 |
| 2759 | CG | GLU | B | 323 | 22.973 | −22.133 | 108.226 | 1.00 | 57.51 |
| 2760 | CD | GLU | B | 323 | 21.495 | −22.487 | 108.369 | 1.00 | 67.14 |
| 2761 | OE1 | GLU | B | 323 | 21.166 | −23.373 | 109.195 | 1.00 | 63.90 |
| 2762 | OE2 | GLU | B | 323 | 20.660 | −21.893 | 107.643 | 1.00 | 71.98 |
| 2763 | C | GLU | B | 323 | 26.012 | −23.162 | 109.548 | 1.00 | 43.21 |
| 2764 | O | GLU | B | 323 | 26.608 | −23.476 | 110.581 | 1.00 | 44.00 |
| 2765 | N | THR | B | 324 | 26.060 | −23.905 | 108.450 | 1.00 | 42.19 |
| 2766 | CA | THR | B | 324 | 26.793 | −25.159 | 108.442 | 1.00 | 40.90 |
| 2767 | CB | THR | B | 324 | 26.243 | −26.152 | 107.364 | 1.00 | 42.53 |
| 2768 | OG1 | THR | B | 324 | 26.829 | −25.852 | 106.089 | 1.00 | 41.85 |
| 2769 | CG2 | THR | B | 324 | 24.737 | −26.050 | 107.234 | 1.00 | 41.53 |
| 2770 | C | THR | B | 324 | 28.262 | −24.893 | 108.101 | 1.00 | 38.75 |
| 2771 | O | THR | B | 324 | 29.147 | −25.720 | 108.378 | 1.00 | 36.54 |
| 2772 | N | GLU | B | 325 | 28.508 | −23.728 | 107.513 | 1.00 | 33.32 |
| 2773 | CA | GLU | B | 325 | 29.842 | −23.357 | 107.061 | 1.00 | 33.46 |
| 2774 | CB | GLU | B | 325 | 30.856 | −23.420 | 108.213 | 1.00 | 23.98 |
| 2775 | CG | GLU | B | 325 | 31.022 | −22.081 | 108.892 | 1.00 | 41.70 |
| 2776 | CD | GLU | B | 325 | 31.807 | −22.141 | 110.193 | 1.00 | 42.71 |
| 2777 | OE1 | GLU | B | 325 | 33.001 | −22.528 | 110.156 | 1.00 | 40.97 |
| 2778 | OE2 | GLU | B | 325 | 31.216 | −21.807 | 111.251 | 1.00 | 38.92 |
| 2779 | C | GLU | B | 325 | 30.295 | −24.247 | 105.867 | 1.00 | 29.73 |
| 2780 | O | GLU | B | 325 | 31.496 | −24.326 | 105.548 | 1.00 | 29.60 |
| 2781 | N | CYS | B | 326 | 29.329 | −24.897 | 105.210 | 1.00 | 19.72 |
| 2782 | CA | CYS | B | 326 | 29.630 | −25.738 | 104.068 | 1.00 | 17.47 |
| 2783 | CB | CYS | B | 326 | 28.948 | −27.087 | 104.197 | 1.00 | 17.62 |
| 2784 | SG | CYS | B | 326 | 29.778 | −28.099 | 105.458 | 1.00 | 19.81 |
| 2785 | C | CYS | B | 326 | 29.282 | −25.132 | 102.735 | 1.00 | 13.98 |
| 2786 | O | CYS | B | 326 | 28.472 | −24.224 | 102.624 | 1.00 | 14.46 |
| 2787 | N | ILE | B | 327 | 29.934 | −25.641 | 101.713 | 1.00 | 13.56 |
| 2788 | CA | ILE | B | 327 | 29.722 | −25.166 | 100.374 | 1.00 | 14.00 |
| 2789 | CB | ILE | B | 327 | 31.105 | −24.880 | 99.722 | 1.00 | 21.76 |
| 2790 | CG2 | ILE | B | 327 | 30.987 | −24.629 | 98.209 | 1.00 | 15.85 |
| 2791 | CG1 | ILE | B | 327 | 31.744 | −23.685 | 100.431 | 1.00 | 19.00 |
| 2792 | CD1 | ILE | B | 327 | 33.200 | −23.498 | 100.053 | 1.00 | 30.37 |
| 2793 | C | ILE | B | 327 | 28.978 | −26.277 | 99.640 | 1.00 | 15.70 |
| 2794 | O | ILE | B | 327 | 29.495 | −27.397 | 99.480 | 1.00 | 17.00 |
| 2795 | N | THR | B | 328 | 27.749 | −26.014 | 99.225 | 1.00 | 12.99 |
| 2796 | CA | THR | B | 328 | 27.042 | −27.061 | 98.498 | 1.00 | 15.42 |
| 2797 | CB | THR | B | 328 | 25.597 | −27.188 | 99.065 | 1.00 | 17.94 |
| 2798 | OG1 | THR | B | 328 | 24.631 | −27.122 | 97.998 | 1.00 | 16.43 |
| 2799 | CG2 | THR | B | 328 | 25.363 | −26.095 | 100.081 | 1.00 | 15.23 |
| 2800 | C | THR | B | 328 | 27.064 | −26.828 | 96.951 | 1.00 | 14.11 |
| 2801 | O | THR | B | 328 | 26.342 | −25.970 | 96.416 | 1.00 | 14.69 |
| 2802 | N | PHE | B | 329 | 27.913 | −27.588 | 96.257 | 1.00 | 17.12 |
| 2803 | CA | PHE | B | 329 | 28.068 | −27.515 | 94.785 | 1.00 | 24.47 |
| 2804 | CB | PHE | B | 329 | 28.948 | −28.686 | 94.274 | 1.00 | 19.96 |
| 2805 | CG | PHE | B | 329 | 30.309 | −28.730 | 94.908 | 1.00 | 11.22 |
| 2806 | CD1 | PHE | B | 329 | 30.773 | −27.638 | 95.651 | 1.00 | 16.78 |
| 2807 | CD2 | PHE | B | 329 | 31.123 | −29.850 | 94.761 | 1.00 | 17.63 |
| 2808 | CE1 | PHE | B | 329 | 32.019 | −27.651 | 96.241 | 1.00 | 12.06 |
| 2809 | CE2 | PHE | B | 329 | 32.388 | −29.900 | 95.340 | 1.00 | 14.62 |
| 2810 | CZ | PHE | B | 329 | 32.834 | −28.783 | 96.089 | 1.00 | 25.43 |
| 2811 | C | PHE | B | 329 | 26.717 | −27.505 | 94.071 | 1.00 | 27.50 |
| 2812 | O | PHE | B | 329 | 26.329 | −26.543 | 93.424 | 1.00 | 31.32 |
| 2813 | N | LEU | B | 330 | 26.005 | −28.603 | 94.162 | 1.00 | 29.49 |
| 2814 | CA | LEU | B | 330 | 24.678 | −28.677 | 93.591 | 1.00 | 32.93 |
| 2815 | CB | LEU | B | 330 | 24.586 | −29.629 | 92.414 | 1.00 | 20.77 |
| 2816 | CG | LEU | B | 330 | 25.611 | −30.766 | 92.428 | 1.00 | 19.16 |
| 2817 | CD1 | LEU | B | 330 | 25.156 | −31.901 | 91.621 | 1.00 | 13.76 |
| 2818 | CD2 | LEU | B | 330 | 26.910 | −30.266 | 91.897 | 1.00 | 18.33 |
| 2819 | C | LEU | B | 330 | 24.084 | −29.384 | 94.724 | 1.00 | 41.67 |
| 2820 | O | LEU | B | 330 | 24.790 | −29.756 | 95.688 | 1.00 | 48.74 |
| 2821 | N | LYS | B | 331 | 22.778 | −29.531 | 94.708 | 1.00 | 46.64 |
| 2822 | CA | LYS | B | 331 | 22.380 | −30.323 | 95.792 | 1.00 | 51.51 |
| 2823 | CB | LYS | B | 331 | 21.170 | −29.901 | 96.632 | 1.00 | 51.06 |
| 2824 | CG | LYS | B | 331 | 21.365 | −28.780 | 97.591 | 1.00 | 53.95 |
| 2825 | CD | LYS | B | 331 | 20.017 | −28.140 | 97.871 | 1.00 | 48.73 |
| 2826 | CE | LYS | B | 331 | 20.173 | −26.727 | 98.404 | 1.00 | 48.40 |
| 2827 | NZ | LYS | B | 331 | 20.371 | −25.761 | 97.305 | 1.00 | 47.10 |
| 2828 | C | LYS | B | 331 | 22.181 | −31.688 | 95.570 | 1.00 | 50.52 |
| 2829 | O | LYS | B | 331 | 21.849 | −32.204 | 94.478 | 1.00 | 46.66 |
| 2830 | N | ASP | B | 332 | 22.887 | −32.032 | 96.624 | 1.00 | 52.67 |
| 2831 | CA | ASP | B | 332 | 23.025 | −33.222 | 97.365 | 1.00 | 55.22 |
| 2832 | CB | ASP | B | 332 | 22.073 | −34.369 | 96.915 | 1.00 | 59.22 |
| 2833 | CG | ASP | B | 332 | 20.592 | −33.897 | 96.918 | 1.00 | 63.62 |
| 2834 | OD1 | ASP | B | 332 | 20.155 | −33.161 | 97.849 | 1.00 | 60.93 |
| 2835 | OD2 | ASP | B | 332 | 19.873 | −34.259 | 95.952 | 1.00 | 61.76 |
| 2836 | C | ASP | B | 332 | 24.404 | −33.483 | 97.729 | 1.00 | 52.77 |
| 2837 | O | ASP | B | 332 | 24.697 | −34.166 | 98.715 | 1.00 | 57.76 |
| 2838 | N | PHE | B | 333 | 25.166 | −32.599 | 97.109 | 1.00 | 45.92 |
| 2839 | CA | PHE | B | 333 | 26.596 | −32.480 | 97.146 | 1.00 | 42.30 |
| 2840 | CB | PHE | B | 333 | 27.042 | −32.335 | 95.715 | 1.00 | 39.83 |
| 2841 | CG | PHE | B | 333 | 26.642 | −33.497 | 94.836 | 1.00 | 39.89 |
| 2842 | CD1 | PHE | B | 333 | 27.585 | −34.434 | 94.498 | 1.00 | 42.14 |
| 2843 | CD2 | PHE | B | 333 | 25.351 | −33.645 | 94.339 | 1.00 | 39.58 |
| 2844 | CE1 | PHE | B | 333 | 27.271 | −35.506 | 93.681 | 1.00 | 34.24 |
| 2845 | CE2 | PHE | B | 333 | 25.033 | −34.713 | 93.526 | 1.00 | 39.73 |
| 2846 | CZ | PHE | B | 333 | 26.006 | −35.644 | 93.199 | 1.00 | 37.98 |
| 2847 | C | PHE | B | 333 | 27.104 | −31.309 | 97.942 | 1.00 | 40.11 |
| 2848 | O | PHE | B | 333 | 27.208 | −30.199 | 97.420 | 1.00 | 42.42 |
| 2849 | N | THR | B | 334 | 27.449 | −31.564 | 99.198 | 1.00 | 37.35 |
| 2850 | CA | THR | B | 334 | 27.939 | −30.488 | 100.056 | 1.00 | 35.15 |
| 2851 | CB | THR | B | 334 | 26.935 | −30.139 | 101.149 | 1.00 | 27.27 |
| 2852 | OG1 | THR | B | 334 | 25.661 | −30.006 | 100.544 | 1.00 | 40.46 |
| 2853 | CG2 | THR | B | 334 | 27.210 | −28.800 | 101.745 | 1.00 | 35.21 |
| 2854 | C | THR | B | 334 | 29.258 | −30.878 | 100.676 | 1.00 | 33.56 |
| 2855 | O | THR | B | 334 | 29.553 | −32.065 | 100.805 | 1.00 | 32.24 |
| 2856 | N | TYR | B | 335 | 30.067 | −29.866 | 101.011 | 1.00 | 30.56 |
| 2857 | CA | TYR | B | 335 | 31.384 | −30.087 | 101.593 | 1.00 | 23.37 |
| 2858 | CB | TYR | B | 335 | 32.419 | −30.188 | 100.488 | 1.00 | 17.76 |
| 2859 | CG | TYR | B | 335 | 31.948 | −31.134 | 99.385 | 1.00 | 22.34 |
| 2860 | CD1 | TYR | B | 335 | 32.229 | −32.504 | 99.434 | 1.00 | 16.82 |
| 2861 | CE1 | TYR | B | 335 | 31.759 | −33.364 | 98.477 | 1.00 | 21.70 |
| 2862 | CD2 | TYR | B | 335 | 31.168 | −30.671 | 98.338 | 1.00 | 14.59 |
| 2863 | CE2 | TYR | B | 335 | 30.678 | −31.533 | 97.389 | 1.00 | 17.96 |
| 2864 | CZ | TYR | B | 335 | 30.975 | −32.868 | 97.452 | 1.00 | 20.27 |
| 2865 | OH | TYR | B | 335 | 30.488 | −33.727 | 96.499 | 1.00 | 12.76 |
| 2866 | C | TYR | B | 335 | 31.769 | −29.007 | 102.572 | 1.00 | 22.39 |
| 2867 | O | TYR | B | 335 | 31.346 | −27.848 | 102.484 | 1.00 | 22.73 |
| 2868 | N | SER | B | 336 | 32.577 | −29.444 | 103.519 | 1.00 | 18.36 |
| 2869 | CA | SER | B | 336 | 33.087 | −28.641 | 104.605 | 1.00 | 17.31 |
| 2870 | CB | SER | B | 336 | 33.167 | −29.511 | 105.826 | 1.00 | 14.17 |
| 2871 | OG | SER | B | 336 | 34.231 | −30.442 | 105.643 | 1.00 | 5.25 |
| 2872 | C | SER | B | 336 | 34.511 | −28.249 | 104.242 | 1.00 | 19.59 |
| 2873 | O | SER | B | 336 | 35.079 | −28.790 | 103.331 | 1.00 | 18.34 |
| 2874 | N | LYS | B | 337 | 35.107 | −27.336 | 104.988 | 1.00 | 24.25 |
| 2875 | CA | LYS | B | 337 | 36.472 | −26.939 | 104.683 | 1.00 | 30.39 |
| 2876 | CB | LYS | B | 337 | 36.974 | −25.910 | 105.704 | 1.00 | 24.85 |
| 2877 | CG | LYS | B | 337 | 38.128 | −25.057 | 105.208 | 1.00 | 24.92 |
| 2878 | CD | LYS | B | 337 | 38.965 | −24.367 | 106.323 | 1.00 | 10.90 |
| 2879 | CE | LYS | B | 337 | 38.155 | −23.365 | 107.092 | 1.00 | 12.99 |
| 2880 | NZ | LYS | B | 337 | 39.074 | −22.574 | 107.894 | 1.00 | 14.94 |
| 2881 | C | LYS | B | 337 | 37.295 | −28.230 | 104.806 | 1.00 | 34.31 |
| 2882 | O | LYS | B | 337 | 38.262 | −28.511 | 104.044 | 1.00 | 39.45 |
| 2883 | N | ASP | B | 338 | 36.871 | −29.039 | 105.763 | 1.00 | 32.24 |
| 2884 | CA | ASP | B | 338 | 37.555 | −30.281 | 106.046 | 1.00 | 29.77 |
| 2885 | CB | ASP | B | 338 | 36.851 | −30.970 | 107.187 | 1.00 | 37.61 |
| 2886 | CG | ASP | B | 338 | 37.724 | −31.967 | 107.868 | 1.00 | 45.54 |
| 2887 | OD1 | ASP | B | 338 | 38.827 | −31.553 | 108.301 | 1.00 | 45.53 |
| 2888 | OD2 | ASP | B | 338 | 37.304 | −33.153 | 107.970 | 1.00 | 53.47 |
| 2889 | C | ASP | B | 338 | 37.604 | −31.197 | 104.858 | 1.00 | 25.03 |
| 2890 | O | ASP | B | 338 | 38.665 | −31.776 | 104.560 | 1.00 | 14.70 |
| 2891 | N | ASP | B | 339 | 36.440 | −31.347 | 104.216 | 1.00 | 20.92 |
| 2892 | CA | ASP | B | 339 | 36.335 | −32.178 | 103.037 | 1.00 | 23.16 |
| 2893 | CB | ASP | B | 339 | 34.939 | −32.097 | 102.433 | 1.00 | 20.17 |
| 2894 | CG | ASP | B | 339 | 33.865 | −32.866 | 103.280 | 1.00 | 27.23 |
| 2895 | OD1 | ASP | B | 339 | 34.021 | −34.070 | 103.623 | 1.00 | 19.08 |
| 2896 | OD2 | ASP | B | 339 | 32.822 | −32.257 | 103.597 | 1.00 | 19.77 |
| 2897 | C | ASP | B | 339 | 37.397 | −31.793 | 102.006 | 1.00 | 24.92 |
| 2898 | O | ASP | B | 339 | 37.956 | −32.655 | 101.325 | 1.00 | 24.48 |
| 2899 | N | PHE | B | 340 | 37.721 | −30.511 | 101.906 | 1.00 | 26.15 |
| 2900 | CA | PHE | B | 340 | 38.754 | −30.115 | 100.952 | 1.00 | 26.11 |
| 2901 | CB | PHE | B | 340 | 38.901 | −28.586 | 100.891 | 1.00 | 29.18 |
| 2902 | CG | PHE | B | 340 | 37.815 | −27.885 | 100.114 | 1.00 | 28.11 |
| 2903 | CD1 | PHE | B | 340 | 38.137 | −26.833 | 99.236 | 1.00 | 22.50 |
| 2904 | CD2 | PHE | B | 340 | 36.476 | −28.254 | 100.255 | 1.00 | 14.50 |
| 2905 | CE1 | PHE | B | 340 | 37.122 | −26.152 | 98.496 | 1.00 | 9.98 |
| 2906 | CE2 | PHE | B | 340 | 35.453 | −27.588 | 99.522 | 1.00 | 12.32 |
| 2907 | CZ | PHE | B | 340 | 35.779 | −26.540 | 98.642 | 1.00 | 14.96 |
| 2908 | C | PHE | B | 340 | 40.104 | −30.744 | 101.346 | 1.00 | 27.23 |
| 2909 | O | PHE | B | 340 | 40.738 | −31.386 | 100.519 | 1.00 | 21.29 |
| 2910 | N | HIS | B | 341 | 40.532 | −30.546 | 102.601 | 1.00 | 29.89 |
| 2911 | CA | HIS | B | 341 | 41.798 | −31.090 | 103.086 | 1.00 | 29.99 |
| 2912 | CB | HIS | B | 341 | 41.871 | −31.061 | 104.616 | 1.00 | 40.75 |
| 2913 | CG | HIS | B | 341 | 42.227 | −29.717 | 105.203 | 1.00 | 52.52 |
| 2914 | CD2 | HIS | B | 341 | 43.428 | −29.152 | 105.504 | 1.00 | 51.07 |
| 2915 | ND1 | HIS | B | 341 | 41.274 | −28.795 | 105.585 | 1.00 | 59.86 |
| 2916 | CE1 | HIS | B | 341 | 41.866 | −27.727 | 106.094 | 1.00 | 55.78 |
| 2917 | NE2 | HIS | B | 341 | 43.175 | −27.918 | 106.055 | 1.00 | 57.87 |
| 2918 | C | HIS | B | 341 | 41.902 | −32.513 | 102.631 | 1.00 | 27.80 |
| 2919 | O | HIS | B | 341 | 42.897 | −32.913 | 102.032 | 1.00 | 34.95 |
| 2920 | N | ARG | B | 342 | 40.848 | −33.279 | 102.890 | 1.00 | 25.91 |
| 2921 | CA | ARG | B | 342 | 40.808 | −34.698 | 102.505 | 1.00 | 21.72 |
| 2922 | CB | ARG | B | 342 | 39.530 | −35.344 | 103.014 | 1.00 | 23.52 |
| 2923 | CG | ARG | B | 342 | 39.552 | −35.591 | 104.518 | 1.00 | 17.95 |
| 2924 | CD | ARG | B | 342 | 38.174 | −35.868 | 105.004 | 1.00 | 16.70 |
| 2925 | NE | ARG | B | 342 | 38.202 | −36.219 | 106.406 | 1.00 | 35.04 |
| 2926 | CZ | ARG | B | 342 | 37.208 | −35.982 | 107.246 | 1.00 | 44.04 |
| 2927 | NH1 | ARG | B | 342 | 36.106 | −35.382 | 106.810 | 1.00 | 44.47 |
| 2928 | NH2 | ARG | B | 342 | 37.328 | −36.359 | 108.517 | 1.00 | 59.88 |
| 2929 | C | ARG | B | 342 | 40.943 | −34.976 | 101.036 | 1.00 | 17.70 |
| 2930 | O | ARG | B | 342 | 41.451 | −36.009 | 100.669 | 1.00 | 12.22 |
| 2931 | N | ALA | B | 343 | 40.496 | −34.037 | 100.206 | 1.00 | 21.91 |
| 2932 | CA | ALA | B | 343 | 40.562 | −34.182 | 98.749 | 1.00 | 22.78 |
| 2933 | CB | ALA | B | 343 | 39.538 | −33.254 | 98.055 | 1.00 | 10.94 |
| 2934 | C | ALA | B | 343 | 41.955 | −33.853 | 98.261 | 1.00 | 26.32 |
| 2935 | O | ALA | B | 343 | 42.180 | −33.763 | 97.044 | 1.00 | 31.56 |
| 2936 | N | GLY | B | 344 | 42.882 | −33.674 | 99.211 | 1.00 | 25.61 |
| 2937 | CA | GLY | B | 344 | 44.261 | −33.350 | 98.868 | 1.00 | 25.49 |
| 2938 | C | GLY | B | 344 | 44.617 | −31.872 | 98.760 | 1.00 | 23.60 |
| 2939 | O | GLY | B | 344 | 45.780 | −31.508 | 98.561 | 1.00 | 26.94 |
| 2940 | N | LEU | B | 345 | 43.631 | −31.002 | 98.884 | 1.00 | 19.91 |
| 2941 | CA | LEU | B | 345 | 43.948 | −29.597 | 98.832 | 1.00 | 21.14 |
| 2942 | CB | LEU | B | 345 | 42.662 | −28.777 | 98.752 | 1.00 | 18.78 |
| 2943 | CG | LEU | B | 345 | 41.720 | −29.293 | 97.655 | 1.00 | 18.00 |
| 2944 | CD1 | LEU | B | 345 | 40.533 | −28.388 | 97.525 | 1.00 | 16.17 |
| 2945 | CD2 | LEU | B | 345 | 42.454 | −29.398 | 96.346 | 1.00 | 17.82 |
| 2946 | C | LEU | B | 345 | 44.808 | −29.150 | 100.031 | 1.00 | 24.55 |
| 2947 | O | LEU | B | 345 | 44.765 | −29.733 | 101.102 | 1.00 | 24.17 |
| 2948 | N | GLN | B | 346 | 45.618 | −28.121 | 99.801 | 1.00 | 31.30 |
| 2949 | CA | GLN | B | 346 | 46.490 | −27.508 | 100.803 | 1.00 | 28.94 |
| 2950 | CB | GLN | B | 346 | 47.551 | −26.644 | 100.128 | 1.00 | 29.62 |
| 2951 | CG | GLN | B | 346 | 48.427 | −27.381 | 99.193 | 1.00 | 35.38 |
| 2952 | CD | GLN | B | 346 | 49.364 | −26.444 | 98.485 | 1.00 | 40.33 |
| 2953 | OE1 | GLN | B | 346 | 48.924 | −25.417 | 97.959 | 1.00 | 47.15 |
| 2954 | NE2 | GLN | B | 346 | 50.664 | −26.781 | 98.460 | 1.00 | 37.94 |
| 2955 | C | GLN | B | 346 | 45.708 | −26.573 | 101.741 | 1.00 | 27.28 |
| 2956 | O | GLN | B | 346 | 44.650 | −26.030 | 101.411 | 1.00 | 27.98 |
| 2957 | N | VAL | B | 347 | 46.287 | −26.330 | 102.900 | 1.00 | 24.67 |
| 2958 | CA | VAL | B | 347 | 45.646 | −25.477 | 103.846 | 1.00 | 18.01 |
| 2959 | CB | VAL | B | 347 | 46.257 | −25.722 | 105.278 | 1.00 | 10.17 |
| 2960 | CG1 | VAL | B | 347 | 47.379 | −24.760 | 105.559 | 1.00 | 10.52 |
| 2961 | CG2 | VAL | B | 347 | 45.188 | −25.644 | 106.323 | 1.00 | 12.51 |
| 2962 | C | VAL | B | 347 | 45.834 | −24.044 | 103.346 | 1.00 | 17.79 |
| 2963 | O | VAL | B | 347 | 45.013 | −23.175 | 103.577 | 1.00 | 22.76 |
| 2964 | N | GLU | B | 348 | 46.899 | −23.796 | 102.605 | 1.00 | 17.73 |
| 2965 | CA | GLU | B | 348 | 47.163 | −22.436 | 102.170 | 1.00 | 13.53 |
| 2966 | CB | GLU | B | 348 | 48.639 | −22.295 | 101.753 | 1.00 | 14.31 |
| 2967 | CG | GLU | B | 348 | 49.467 | −23.583 | 101.838 | 1.00 | 31.23 |
| 2968 | CD | GLU | B | 348 | 50.376 | −23.631 | 103.046 | 1.00 | 44.88 |
| 2969 | OE1 | GLU | B | 348 | 49.869 | −23.564 | 104.198 | 1.00 | 51.92 |
| 2970 | OE2 | GLU | B | 348 | 51.613 | −23.725 | 102.843 | 1.00 | 50.16 |
| 2971 | C | GLU | B | 348 | 46.245 | −22.001 | 101.060 | 1.00 | 13.72 |
| 2972 | O | GLU | B | 348 | 46.186 | −20.826 | 100.716 | 1.00 | 15.06 |
| 2973 | N | PHE | B | 349 | 45.531 | −22.950 | 100.476 | 1.00 | 16.82 |
| 2974 | CA | PHE | B | 349 | 44.587 | −22.625 | 99.406 | 1.00 | 13.74 |
| 2975 | CB | PHE | B | 349 | 44.708 | −23.669 | 98.281 | 1.00 | 18.78 |
| 2976 | CG | PHE | B | 349 | 43.740 | −23.473 | 97.114 | 1.00 | 10.99 |
| 2977 | CD1 | PHE | B | 349 | 43.459 | −22.220 | 96.614 | 1.00 | 18.13 |
| 2978 | CD2 | PHE | B | 349 | 43.132 | −24.560 | 96.507 | 1.00 | 13.56 |
| 2979 | CE1 | PHE | B | 349 | 42.573 | −22.050 | 95.525 | 1.00 | 6.01 |
| 2980 | CE2 | PHE | B | 349 | 42.255 | −24.397 | 95.419 | 1.00 | 5.27 |
| 2981 | CZ | PHE | B | 349 | 41.981 | −23.144 | 94.937 | 1.00 | 5.69 |
| 2982 | C | PHE | B | 349 | 43.193 | −22.668 | 100.063 | 1.00 | 15.65 |
| 2983 | O | PHE | B | 349 | 42.428 | −21.690 | 100.012 | 1.00 | 13.18 |
| 2984 | N | ILE | B | 350 | 42.888 | −23.793 | 100.719 | 1.00 | 18.64 |
| 2985 | CA | ILE | B | 350 | 41.599 | −24.029 | 101.411 | 1.00 | 15.73 |
| 2986 | CB | ILE | B | 350 | 41.711 | −25.319 | 102.245 | 1.00 | 15.61 |
| 2987 | CG2 | ILE | B | 350 | 40.518 | −25.557 | 103.096 | 1.00 | 19.07 |
| 2988 | CG1 | ILE | B | 350 | 41.814 | −26.498 | 101.316 | 1.00 | 17.50 |
| 2989 | CD1 | ILE | B | 350 | 42.135 | −27.753 | 102.103 | 1.00 | 27.64 |
| 2990 | C | ILE | B | 350 | 41.176 | −22.844 | 102.296 | 1.00 | 17.73 |
| 2991 | O | ILE | B | 350 | 40.153 | −22.176 | 102.039 | 1.00 | 17.58 |
| 2992 | N | ASN | B | 351 | 41.964 | −22.558 | 103.329 | 1.00 | 15.36 |
| 2993 | CA | ASN | B | 351 | 41.630 | −21.426 | 104.200 | 1.00 | 21.50 |
| 2994 | CB | ASN | B | 351 | 42.773 | −21.092 | 105.184 | 1.00 | 18.55 |
| 2995 | CG | ASN | B | 351 | 43.060 | −22.266 | 106.118 | 1.00 | 33.34 |
| 2996 | OD1 | ASN | B | 351 | 42.180 | −23.130 | 106.341 | 1.00 | 30.31 |
| 2997 | ND2 | ASN | B | 351 | 44.276 | −22.324 | 106.657 | 1.00 | 32.74 |
| 2998 | C | ASN | B | 351 | 41.200 | −20.192 | 103.432 | 1.00 | 17.98 |
| 2999 | O | ASN | B | 351 | 40.127 | −19.675 | 103.672 | 1.00 | 19.95 |
| 3000 | N | PRO | B | 352 | 42.023 | −19.723 | 102.498 | 1.00 | 17.67 |
| 3001 | CD | PRO | B | 352 | 43.393 | −20.189 | 102.230 | 1.00 | 16.16 |
| 3002 | CA | PRO | B | 352 | 41.709 | −18.543 | 101.689 | 1.00 | 17.00 |
| 3003 | CB | PRO | B | 352 | 42.870 | −18.483 | 100.713 | 1.00 | 11.78 |
| 3004 | CG | PRO | B | 352 | 43.981 | −18.986 | 101.503 | 1.00 | 13.56 |
| 3005 | C | PRO | B | 352 | 40.358 | −18.651 | 100.942 | 1.00 | 18.90 |
| 3006 | O | PRO | B | 352 | 39.594 | −17.663 | 100.873 | 1.00 | 15.40 |
| 3007 | N | ILE | B | 353 | 40.022 | −19.835 | 100.415 | 1.00 | 15.19 |
| 3008 | CA | ILE | B | 353 | 38.747 | −19.892 | 99.693 | 1.00 | 16.53 |
| 3009 | CB | ILE | B | 353 | 38.596 | −21.164 | 98.749 | 1.00 | 13.88 |
| 3010 | CG2 | ILE | B | 353 | 39.954 | −21.816 | 98.408 | 1.00 | 26.94 |
| 3011 | CG1 | ILE | B | 353 | 37.724 | −22.179 | 99.393 | 1.00 | 33.16 |
| 3012 | CD1 | ILE | B | 353 | 36.288 | −21.967 | 99.061 | 1.00 | 45.07 |
| 3013 | C | ILE | B | 353 | 37.579 | −19.748 | 100.646 | 1.00 | 11.77 |
| 3014 | O | ILE | B | 353 | 36.557 | −19.178 | 100.319 | 1.00 | 17.06 |
| 3015 | N | PHE | B | 354 | 37.728 | −20.236 | 101.856 | 1.00 | 13.84 |
| 3016 | CA | PHE | B | 354 | 36.629 | −20.093 | 102.813 | 1.00 | 16.88 |
| 3017 | CB | PHE | B | 354 | 36.720 | −21.198 | 103.879 | 1.00 | 14.93 |
| 3018 | CG | PHE | B | 354 | 36.075 | −22.477 | 103.456 | 1.00 | 16.09 |
| 3019 | CD1 | PHE | B | 354 | 36.685 | −23.305 | 102.518 | 1.00 | 24.52 |
| 3020 | CD2 | PHE | B | 354 | 34.824 | −22.815 | 103.921 | 1.00 | 18.19 |
| 3021 | CE1 | PHE | B | 354 | 36.057 | −24.442 | 102.041 | 1.00 | 12.44 |
| 3022 | CE2 | PHE | B | 354 | 34.177 | −23.952 | 103.450 | 1.00 | 10.08 |
| 3023 | CZ | PHE | B | 354 | 34.797 | −24.755 | 102.515 | 1.00 | 20.01 |
| 3024 | C | PHE | B | 354 | 36.543 | −18.679 | 103.467 | 1.00 | 16.92 |
| 3025 | O | PHE | B | 354 | 35.533 | −18.254 | 104.046 | 1.00 | 8.38 |
| 3026 | N | GLU | B | 355 | 37.604 | −17.921 | 103.338 | 1.00 | 20.86 |
| 3027 | CA | GLU | B | 355 | 37.561 | −16.616 | 103.904 | 1.00 | 24.71 |
| 3028 | CB | GLU | B | 355 | 38.979 | −16.173 | 104.263 | 1.00 | 26.64 |
| 3029 | CG | GLU | B | 355 | 39.198 | −15.870 | 105.748 | 1.00 | 45.42 |
| 3030 | CD | GLU | B | 355 | 40.442 | −16.586 | 106.324 | 1.00 | 62.33 |
| 3031 | OE1 | GLU | B | 355 | 40.307 | −17.709 | 106.894 | 1.00 | 72.73 |
| 3032 | OE2 | GLU | B | 355 | 41.565 | −16.039 | 106.187 | 1.00 | 65.39 |
| 3033 | C | GLU | B | 355 | 36.916 | −15.726 | 102.813 | 1.00 | 25.19 |
| 3034 | O | GLU | B | 355 | 36.197 | −14.750 | 103.097 | 1.00 | 24.85 |
| 3035 | N | PHE | B | 356 | 37.148 | −16.085 | 101.560 | 1.00 | 19.78 |
| 3036 | CA | PHE | B | 356 | 36.585 | −15.306 | 100.472 | 1.00 | 17.79 |
| 3037 | CB | PHE | B | 356 | 37.097 | −15.856 | 99.132 | 1.00 | 18.91 |
| 3038 | CG | PHE | B | 356 | 36.512 | −15.196 | 97.925 | 1.00 | 14.83 |
| 3039 | CD1 | PHE | B | 356 | 36.826 | −13.860 | 97.610 | 1.00 | 5.25 |
| 3040 | CD2 | PHE | B | 356 | 35.707 | −15.932 | 97.058 | 1.00 | 9.68 |
| 3041 | CE1 | PHE | B | 356 | 36.349 | −13.276 | 96.451 | 1.00 | 5.25 |
| 3042 | CE2 | PHE | B | 356 | 35.217 | −15.346 | 95.869 | 1.00 | 12.90 |
| 3043 | CZ | PHE | B | 356 | 35.551 | −14.009 | 95.576 | 1.00 | 7.76 |
| 3044 | C | PHE | B | 356 | 35.063 | −15.423 | 100.525 | 1.00 | 17.85 |
| 3045 | O | PHE | B | 356 | 34.336 | −14.417 | 100.378 | 1.00 | 13.00 |
| 3046 | N | SER | B | 357 | 34.600 | −16.664 | 100.715 | 1.00 | 17.34 |
| 3047 | CA | SER | B | 357 | 33.182 | −16.957 | 100.782 | 1.00 | 16.99 |
| 3048 | CB | SER | B | 357 | 32.961 | −18.441 | 101.009 | 1.00 | 22.49 |
| 3049 | OG | SER | B | 357 | 33.091 | −19.171 | 99.819 | 1.00 | 31.90 |
| 3050 | C | SER | B | 357 | 32.586 | −16.187 | 101.935 | 1.00 | 16.72 |
| 3051 | O | SER | B | 357 | 31.539 | −15.548 | 101.801 | 1.00 | 15.43 |
| 3052 | N | ARG | B | 358 | 33.255 | −16.250 | 103.085 | 1.00 | 18.90 |
| 3053 | CA | ARG | B | 358 | 32.755 | −15.521 | 104.240 | 1.00 | 18.40 |
| 3054 | CB | ARG | B | 358 | 33.647 | −15.774 | 105.467 | 1.00 | 14.72 |
| 3055 | CG | ARG | B | 358 | 33.420 | −17.131 | 106.105 | 1.00 | 7.82 |
| 3056 | CD | ARG | B | 358 | 34.071 | −17.220 | 107.487 | 1.00 | 8.08 |
| 3057 | NE | ARG | B | 358 | 35.545 | −17.239 | 107.462 | 1.00 | 17.36 |
| 3058 | CZ | ARG | B | 358 | 36.292 | −18.310 | 107.173 | 1.00 | 26.94 |
| 3059 | NH1 | ARG | B | 358 | 35.732 | −19.490 | 106.876 | 1.00 | 31.94 |
| 3060 | NH2 | ARG | B | 358 | 37.612 | −18.205 | 107.178 | 1.00 | 20.34 |
| 3061 | C | ARG | B | 358 | 32.640 | −14.022 | 103.896 | 1.00 | 15.62 |
| 3062 | O | ARG | B | 358 | 31.602 | −13.371 | 104.137 | 1.00 | 19.53 |
| 3063 | N | ALA | B | 359 | 33.686 | −13.469 | 103.308 | 1.00 | 12.15 |
| 3064 | CA | ALA | B | 359 | 33.619 | −12.061 | 102.903 | 1.00 | 18.05 |
| 3065 | CB | ALA | B | 359 | 34.972 | −11.565 | 102.396 | 1.00 | 8.61 |
| 3066 | C | ALA | B | 359 | 32.536 | −11.765 | 101.856 | 1.00 | 19.02 |
| 3067 | O | ALA | B | 359 | 31.962 | −10.688 | 101.883 | 1.00 | 18.91 |
| 3068 | N | MET | B | 360 | 32.254 | −12.699 | 100.948 | 1.00 | 17.99 |
| 3069 | CA | MET | B | 360 | 31.214 | −12.460 | 99.961 | 1.00 | 22.61 |
| 3070 | CB | MET | B | 360 | 31.225 | −13.523 | 98.842 | 1.00 | 26.94 |
| 3071 | CG | MET | B | 360 | 32.423 | −13.472 | 97.882 | 1.00 | 32.65 |
| 3072 | SD | MET | B | 360 | 32.647 | −11.916 | 96.899 | 1.00 | 18.14 |
| 3073 | CE | MET | B | 360 | 31.021 | −11.313 | 96.962 | 1.00 | 24.58 |
| 3074 | C | MET | B | 360 | 29.837 | −12.462 | 100.634 | 1.00 | 26.84 |
| 3075 | O | MET | B | 360 | 28.944 | −11.707 | 100.227 | 1.00 | 31.39 |
| 3076 | N | ARG | B | 361 | 29.633 | −13.334 | 101.622 | 1.00 | 26.66 |
| 3077 | CA | ARG | B | 361 | 28.359 | −13.361 | 102.321 | 1.00 | 27.45 |
| 3078 | CB | ARG | B | 361 | 28.368 | −14.465 | 103.342 | 1.00 | 32.86 |
| 3079 | CG | ARG | B | 361 | 27.103 | −14.454 | 104.197 | 1.00 | 48.02 |
| 3080 | CD | ARG | B | 361 | 27.327 | −15.124 | 105.526 | 1.00 | 50.09 |
| 3081 | NE | ARG | B | 361 | 26.385 | −14.640 | 106.520 | 1.00 | 53.80 |
| 3082 | CZ | ARG | B | 361 | 25.103 | −14.970 | 106.558 | 1.00 | 61.07 |
| 3083 | NH1 | ARG | B | 361 | 24.602 | −15.792 | 105.648 | 1.00 | 49.75 |
| 3084 | NH2 | ARG | B | 361 | 24.336 | −14.487 | 107.529 | 1.00 | 70.63 |
| 3085 | C | ARG | B | 361 | 28.136 | −12.011 | 103.039 | 1.00 | 30.85 |
| 3086 | O | ARG | B | 361 | 26.998 | −11.534 | 103.201 | 1.00 | 28.80 |
| 3087 | N | ARG | B | 362 | 29.241 | −11.413 | 103.479 | 1.00 | 33.49 |
| 3088 | CA | ARG | B | 362 | 29.244 | −10.115 | 104.142 | 1.00 | 34.01 |
| 3089 | CB | ARG | B | 362 | 30.661 | −9.784 | 104.607 | 1.00 | 41.72 |
| 3090 | CG | ARG | B | 362 | 31.017 | −10.242 | 106.011 | 1.00 | 51.35 |
| 3091 | CD | ARG | B | 362 | 32.398 | −9.709 | 106.379 | 1.00 | 58.26 |
| 3092 | NE | ARG | B | 362 | 33.420 | −10.743 | 106.288 | 1.00 | 53.85 |
| 3093 | CZ | ARG | B | 362 | 34.679 | −10.504 | 105.961 | 1.00 | 56.47 |
| 3094 | NH1 | ARG | B | 362 | 35.054 | −9.255 | 105.687 | 1.00 | 46.23 |
| 3095 | NH2 | ARG | B | 362 | 35.541 | −11.522 | 105.929 | 1.00 | 59.73 |
| 3096 | C | ARG | B | 362 | 28.764 | −8.970 | 103.249 | 1.00 | 34.69 |
| 3097 | O | ARG | B | 362 | 28.657 | −7.836 | 103.722 | 1.00 | 40.31 |
| 3098 | N | LEU | B | 363 | 28.525 | −9.236 | 101.962 | 1.00 | 30.88 |
| 3099 | CA | LEU | B | 363 | 28.057 | −8.179 | 101.042 | 1.00 | 28.18 |
| 3100 | CB | LEU | B | 363 | 28.981 | −8.014 | 99.833 | 1.00 | 26.95 |
| 3101 | CG | LEU | B | 363 | 30.405 | −7.667 | 100.163 | 1.00 | 27.79 |
| 3102 | CD1 | LEU | B | 363 | 31.253 | −7.675 | 98.903 | 1.00 | 32.32 |
| 3103 | CD2 | LEU | B | 363 | 30.410 | −6.323 | 100.820 | 1.00 | 30.61 |
| 3104 | C | LEU | B | 363 | 26.657 | −8.448 | 100.550 | 1.00 | 22.85 |
| 3105 | O | LEU | B | 363 | 26.039 | −7.540 | 99.992 | 1.00 | 21.59 |
| 3106 | N | GLY | B | 364 | 26.209 | −9.693 | 100.747 | 1.00 | 17.08 |
| 3107 | CA | GLY | B | 364 | 24.877 | −10.120 | 100.391 | 1.00 | 16.34 |
| 3108 | C | GLY | B | 364 | 24.509 | −9.830 | 98.972 | 1.00 | 16.19 |
| 3109 | O | GLY | B | 364 | 23.456 | −9.193 | 98.727 | 1.00 | 12.85 |
| 3110 | N | LEU | B | 365 | 25.367 | −10.296 | 98.057 | 1.00 | 13.81 |
| 3111 | CA | LEU | B | 365 | 25.139 | −10.118 | 96.630 | 1.00 | 13.79 |
| 3112 | CB | LEU | B | 365 | 26.407 | −10.484 | 95.853 | 1.00 | 14.20 |
| 3113 | CG | LEU | B | 365 | 27.688 | −9.876 | 96.405 | 1.00 | 21.72 |
| 3114 | CD1 | LEU | B | 365 | 28.706 | −9.735 | 95.330 | 1.00 | 31.54 |
| 3115 | CD2 | LEU | B | 365 | 27.391 | −8.524 | 96.953 | 1.00 | 39.02 |
| 3116 | C | LEU | B | 365 | 23.974 | −10.995 | 96.179 | 1.00 | 13.42 |
| 3117 | O | LEU | B | 365 | 23.917 | −12.142 | 96.546 | 1.00 | 13.34 |
| 3118 | N | ASP | B | 366 | 23.039 | −10.443 | 95.394 | 1.00 | 19.07 |
| 3119 | CA | ASP | B | 366 | 21.862 | −11.184 | 94.884 | 1.00 | 16.89 |
| 3120 | CB | ASP | B | 366 | 20.668 | −10.270 | 94.648 | 1.00 | 13.26 |
| 3121 | CG | ASP | B | 366 | 20.962 | −9.161 | 93.670 | 1.00 | 23.83 |
| 3122 | OD1 | ASP | B | 366 | 21.482 | −9.459 | 92.574 | 1.00 | 26.79 |
| 3123 | OD2 | ASP | B | 366 | 20.653 | −7.987 | 93.996 | 1.00 | 36.61 |
| 3124 | C | ASP | B | 366 | 22.213 | −11.842 | 93.575 | 1.00 | 19.58 |
| 3125 | O | ASP | B | 366 | 23.296 | −11.576 | 93.006 | 1.00 | 13.52 |
| 3126 | N | ASP | B | 367 | 21.298 | −12.680 | 93.087 | 1.00 | 20.02 |
| 3127 | CA | ASP | B | 367 | 21.518 | −13.426 | 91.839 | 1.00 | 22.33 |
| 3128 | CB | ASP | B | 367 | 20.271 | −14.245 | 91.504 | 1.00 | 24.27 |
| 3129 | CG | ASP | B | 367 | 19.890 | −15.194 | 92.616 | 1.00 | 43.14 |
| 3130 | OD1 | ASP | B | 367 | 20.540 | −16.280 | 92.703 | 1.00 | 53.43 |
| 3131 | OD2 | ASP | B | 367 | 18.958 | −14.843 | 93.413 | 1.00 | 48.18 |
| 3132 | C | ASP | B | 367 | 21.943 | −12.596 | 90.604 | 1.00 | 19.76 |
| 3133 | O | ASP | B | 367 | 22.597 | −13.127 | 89.719 | 1.00 | 11.62 |
| 3134 | N | ALA | B | 368 | 21.581 | −11.308 | 90.564 | 1.00 | 19.98 |
| 3135 | CA | ALA | B | 368 | 21.918 | −10.406 | 89.458 | 1.00 | 19.72 |
| 3136 | CB | ALA | B | 368 | 20.915 | −9.338 | 89.372 | 1.00 | 15.55 |
| 3137 | C | ALA | B | 368 | 23.295 | −9.774 | 89.595 | 1.00 | 22.54 |
| 3138 | O | ALA | B | 368 | 23.994 | −9.511 | 88.596 | 1.00 | 22.54 |
| 3139 | N | GLU | B | 369 | 23.677 | −9.504 | 90.840 | 1.00 | 23.40 |
| 3140 | CA | GLU | B | 369 | 24.980 | −8.925 | 91.113 | 1.00 | 20.72 |
| 3141 | CB | GLU | B | 369 | 25.057 | −8.470 | 92.564 | 1.00 | 17.66 |
| 3142 | CG | GLU | B | 369 | 23.891 | −7.514 | 92.884 | 1.00 | 25.36 |
| 3143 | CD | GLU | B | 369 | 23.962 | −6.878 | 94.263 | 1.00 | 29.99 |
| 3144 | OE1 | GLU | B | 369 | 24.111 | −7.633 | 95.253 | 1.00 | 26.69 |
| 3145 | OE2 | GLU | B | 369 | 23.866 | −5.627 | 94.362 | 1.00 | 16.79 |
| 3146 | C | GLU | B | 369 | 26.028 | −9.960 | 90.769 | 1.00 | 18.11 |
| 3147 | O | GLU | B | 369 | 27.003 | −9.670 | 90.085 | 1.00 | 20.17 |
| 3148 | N | TYR | B | 370 | 25.799 | −11.195 | 91.178 | 1.00 | 18.30 |
| 3149 | CA | TYR | B | 370 | 26.749 | −12.237 | 90.852 | 1.00 | 15.68 |
| 3150 | CB | TYR | B | 370 | 26.302 | −13.623 | 91.353 | 1.00 | 19.37 |
| 3151 | CG | TYR | B | 370 | 26.827 | −14.016 | 92.727 | 1.00 | 7.23 |
| 3152 | CD1 | TYR | B | 370 | 26.032 | −13.928 | 93.846 | 1.00 | 17.22 |
| 3153 | CE1 | TYR | B | 370 | 26.514 | −14.243 | 95.109 | 1.00 | 15.61 |
| 3154 | CD2 | TYR | B | 370 | 28.116 | −14.429 | 92.895 | 1.00 | 9.51 |
| 3155 | CE2 | TYR | B | 370 | 28.614 | −14.734 | 94.142 | 1.00 | 15.58 |
| 3156 | CZ | TYR | B | 370 | 27.819 | −14.637 | 95.246 | 1.00 | 14.19 |
| 3157 | OH | TYR | B | 370 | 28.371 | −14.858 | 96.486 | 1.00 | 24.16 |
| 3158 | C | TYR | B | 370 | 26.898 | −12.270 | 89.358 | 1.00 | 12.51 |
| 3159 | O | TYR | B | 370 | 27.972 | −12.022 | 88.827 | 1.00 | 17.46 |
| 3160 | N | ALA | B | 371 | 25.815 | −12.556 | 88.665 | 1.00 | 11.93 |
| 3161 | CA | ALA | B | 371 | 25.889 | −12.626 | 87.217 | 1.00 | 14.57 |
| 3162 | CB | ALA | B | 371 | 24.497 | −12.590 | 86.620 | 1.00 | 5.25 |
| 3163 | C | ALA | B | 371 | 26.755 | −11.477 | 86.681 | 1.00 | 17.10 |
| 3164 | O | ALA | B | 371 | 27.791 | −11.716 | 85.993 | 1.00 | 16.34 |
| 3165 | N | LEU | B | 372 | 26.372 | −10.242 | 87.028 | 1.00 | 17.56 |
| 3166 | CA | LEU | B | 372 | 27.135 | −9.070 | 86.558 | 1.00 | 18.82 |
| 3167 | CB | LEU | B | 372 | 26.540 | −7.775 | 87.107 | 1.00 | 13.33 |
| 3168 | CG | LEU | B | 372 | 25.210 | −7.389 | 86.494 | 1.00 | 12.05 |
| 3169 | CD1 | LEU | B | 372 | 24.794 | −6.048 | 87.024 | 1.00 | 15.59 |
| 3170 | CD2 | LEU | B | 372 | 25.382 | −7.304 | 84.965 | 1.00 | 16.90 |
| 3171 | C | LEU | B | 372 | 28.625 | −9.147 | 86.917 | 1.00 | 16.85 |
| 3172 | O | LEU | B | 372 | 29.492 | −8.975 | 86.048 | 1.00 | 13.14 |
| 3173 | N | LEU | B | 373 | 28.910 | −9.444 | 88.185 | 1.00 | 13.85 |
| 3174 | CA | LEU | B | 373 | 30.281 | −9.548 | 88.629 | 1.00 | 12.62 |
| 3175 | CB | LEU | B | 373 | 30.329 | −9.940 | 90.118 | 1.00 | 7.59 |
| 3176 | CG | LEU | B | 373 | 31.566 | −9.456 | 90.844 | 1.00 | 8.37 |
| 3177 | CD1 | LEU | B | 373 | 31.398 | −7.985 | 91.121 | 1.00 | 9.21 |
| 3178 | CD2 | LEU | B | 373 | 31.715 | −10.206 | 92.083 | 1.00 | 5.25 |
| 3179 | C | LEU | B | 373 | 31.023 | −10.587 | 87.762 | 1.00 | 11.70 |
| 3180 | O | LEU | B | 373 | 32.143 | −10.350 | 87.336 | 1.00 | 13.99 |
| 3181 | N | ILE | B | 374 | 30.400 | −11.735 | 87.504 | 1.00 | 12.16 |
| 3182 | CA | ILE | B | 374 | 31.028 | −12.752 | 86.677 | 1.00 | 9.86 |
| 3183 | CB | ILE | B | 374 | 30.151 | −14.010 | 86.561 | 1.00 | 5.59 |
| 3184 | CG2 | ILE | B | 374 | 30.777 | −15.013 | 85.607 | 1.00 | 5.25 |
| 3185 | CG1 | ILE | B | 374 | 30.042 | −14.676 | 87.918 | 1.00 | 10.39 |
| 3186 | CD1 | ILE | B | 374 | 29.306 | −16.037 | 87.893 | 1.00 | 7.46 |
| 3187 | C | ILE | B | 374 | 31.337 | −12.221 | 85.259 | 1.00 | 14.32 |
| 3188 | O | ILE | B | 374 | 32.444 | −12.492 | 84.724 | 1.00 | 11.00 |
| 3189 | N | ALA | B | 375 | 30.387 | −11.479 | 84.658 | 1.00 | 13.12 |
| 3190 | CA | ALA | B | 375 | 30.627 | −10.941 | 83.334 | 1.00 | 13.52 |
| 3191 | CB | ALA | B | 375 | 29.407 | −10.217 | 82.820 | 1.00 | 20.18 |
| 3192 | C | ALA | B | 375 | 31.818 | −9.980 | 83.427 | 1.00 | 14.33 |
| 3193 | O | ALA | B | 375 | 32.787 | −10.096 | 82.677 | 1.00 | 19.14 |
| 3194 | N | ILE | B | 376 | 31.753 | −9.030 | 84.351 | 1.00 | 14.01 |
| 3195 | CA | ILE | B | 376 | 32.851 | −8.085 | 84.524 | 1.00 | 11.25 |
| 3196 | CB | ILE | B | 376 | 32.680 | −7.249 | 85.834 | 1.00 | 8.47 |
| 3197 | CG2 | ILE | B | 376 | 33.993 | −6.556 | 86.203 | 1.00 | 13.86 |
| 3198 | CG1 | ILE | B | 376 | 31.583 | −6.193 | 85.633 | 1.00 | 7.99 |
| 3199 | CD1 | ILE | B | 376 | 31.150 | −5.454 | 86.914 | 1.00 | 12.21 |
| 3200 | C | ILE | B | 376 | 34.153 | −8.860 | 84.581 | 1.00 | 9.44 |
| 3201 | O | ILE | B | 376 | 35.108 | −8.589 | 83.860 | 1.00 | 11.32 |
| 3202 | N | ASN | B | 377 | 34.183 | −9.848 | 85.452 | 1.00 | 12.26 |
| 3203 | CA | ASN | B | 377 | 35.359 | −10.671 | 85.620 | 1.00 | 11.47 |
| 3204 | CB | ASN | B | 377 | 35.061 | −11.776 | 86.624 | 1.00 | 9.20 |
| 3205 | CG | ASN | B | 377 | 36.219 | −12.689 | 86.832 | 1.00 | 7.74 |
| 3206 | OD1 | ASN | B | 377 | 36.523 | −13.554 | 86.005 | 1.00 | 23.45 |
| 3207 | ND2 | ASN | B | 377 | 36.886 | −12.512 | 87.940 | 1.00 | 13.06 |
| 3208 | C | ASN | B | 377 | 35.831 | −11.254 | 84.299 | 1.00 | 12.17 |
| 3209 | O | ASN | B | 377 | 36.985 | −11.166 | 84.000 | 1.00 | 15.74 |
| 3210 | N | ILE | B | 378 | 34.947 | −11.869 | 83.517 | 1.00 | 17.76 |
| 3211 | CA | ILE | B | 378 | 35.327 | −12.466 | 82.214 | 1.00 | 16.66 |
| 3212 | CB | ILE | B | 378 | 34.124 | −12.997 | 81.466 | 1.00 | 9.28 |
| 3213 | CG2 | ILE | B | 378 | 34.549 | −13.578 | 80.132 | 1.00 | 13.78 |
| 3214 | CG1 | ILE | B | 378 | 33.475 | −14.086 | 82.288 | 1.00 | 7.47 |
| 3215 | CD1 | ILE | B | 378 | 32.276 | −14.711 | 81.563 | 1.00 | 14.99 |
| 3216 | C | ILE | B | 378 | 36.021 | −11.473 | 81.284 | 1.00 | 16.69 |
| 3217 | O | ILE | B | 378 | 37.026 | −11.816 | 80.626 | 1.00 | 17.57 |
| 3218 | N | PHE | B | 379 | 35.483 | −10.256 | 81.242 | 1.00 | 11.33 |
| 3219 | CA | PHE | B | 379 | 36.071 | −9.198 | 80.440 | 1.00 | 16.27 |
| 3220 | CB | PHE | B | 379 | 34.967 | −8.240 | 79.995 | 1.00 | 11.04 |
| 3221 | CG | PHE | B | 379 | 33.936 | −8.871 | 79.063 | 1.00 | 13.52 |
| 3222 | CD1 | PHE | B | 379 | 34.322 | −9.497 | 77.873 | 1.00 | 11.53 |
| 3223 | CD2 | PHE | B | 379 | 32.572 | −8.842 | 79.391 | 1.00 | 12.09 |
| 3224 | CE1 | PHE | B | 379 | 33.354 | −10.109 | 77.000 | 1.00 | 5.76 |
| 3225 | CE2 | PHE | B | 379 | 31.627 | −9.420 | 78.570 | 1.00 | 11.76 |
| 3226 | CZ | PHE | B | 379 | 32.029 | −10.073 | 77.341 | 1.00 | 8.68 |
| 3227 | C | PHE | B | 379 | 37.258 | −8.419 | 81.088 | 1.00 | 14.55 |
| 3228 | O | PHE | B | 379 | 37.265 | −7.178 | 81.133 | 1.00 | 14.20 |
| 3229 | N | SER | B | 380 | 38.258 | −9.163 | 81.564 | 1.00 | 16.28 |
| 3230 | CA | SER | B | 380 | 39.459 | −8.574 | 82.167 | 1.00 | 19.10 |
| 3231 | CB | SER | B | 380 | 40.137 | −9.553 | 83.122 | 1.00 | 19.33 |
| 3232 | OG | SER | B | 380 | 39.358 | −9.913 | 84.218 | 1.00 | 15.59 |
| 3233 | C | SER | B | 380 | 40.488 | −8.236 | 81.063 | 1.00 | 21.09 |
| 3234 | O | SER | B | 380 | 41.286 | −9.075 | 80.621 | 1.00 | 17.26 |
| 3235 | N | ALA | B | 381 | 40.483 | −7.002 | 80.605 | 1.00 | 24.34 |
| 3236 | CA | ALA | B | 381 | 41.439 | −6.620 | 79.562 | 1.00 | 25.10 |
| 3237 | CB | ALA | B | 381 | 41.296 | −5.095 | 79.277 | 1.00 | 25.42 |
| 3238 | C | ALA | B | 381 | 42.922 | −6.974 | 79.827 | 1.00 | 18.35 |
| 3239 | O | ALA | B | 381 | 43.728 | −7.012 | 78.913 | 1.00 | 25.10 |
| 3240 | N | ASP | B | 382 | 43.284 | −7.252 | 81.067 | 1.00 | 15.56 |
| 3241 | CA | ASP | B | 382 | 44.693 | −7.524 | 81.396 | 1.00 | 18.77 |
| 3242 | CB | ASP | B | 382 | 45.111 | −6.726 | 82.654 | 1.00 | 10.75 |
| 3243 | CG | ASP | B | 382 | 44.614 | −7.355 | 83.958 | 1.00 | 23.74 |
| 3244 | OD1 | ASP | B | 382 | 43.539 | −7.994 | 83.980 | 1.00 | 24.72 |
| 3245 | OD2 | ASP | B | 382 | 45.298 | −7.202 | 84.991 | 1.00 | 26.03 |
| 3246 | C | ASP | B | 382 | 45.101 | −8.982 | 81.590 | 1.00 | 20.40 |
| 3247 | O | ASP | B | 382 | 46.159 | −9.260 | 82.165 | 1.00 | 13.95 |
| 3248 | N | ARG | B | 383 | 44.257 | −9.910 | 81.130 | 1.00 | 26.01 |
| 3249 | CA | ARG | B | 383 | 44.552 | −11.339 | 81.283 | 1.00 | 25.61 |
| 3250 | CB | ARG | B | 383 | 43.355 | −12.220 | 80.901 | 1.00 | 25.21 |
| 3251 | CG | ARG | B | 383 | 42.121 | −12.108 | 81.792 | 1.00 | 27.60 |
| 3252 | CD | ARG | B | 383 | 42.392 | −12.504 | 83.244 | 1.00 | 31.36 |
| 3253 | NE | ARG | B | 383 | 41.145 | −12.534 | 84.004 | 1.00 | 25.93 |
| 3254 | CZ | ARG | B | 383 | 41.066 | −12.723 | 85.314 | 1.00 | 23.67 |
| 3255 | NH1 | ARG | B | 383 | 42.182 | −12.906 | 86.033 | 1.00 | 13.10 |
| 3256 | NH2 | ARG | B | 383 | 39.855 | −12.713 | 85.886 | 1.00 | 8.01 |
| 3257 | C | ARG | B | 383 | 45.736 | −11.689 | 80.405 | 1.00 | 22.00 |
| 3258 | O | ARG | B | 383 | 46.011 | −11.017 | 79.408 | 1.00 | 20.47 |
| 3259 | N | PRO | B | 384 | 46.486 | −12.725 | 80.794 | 1.00 | 23.24 |
| 3260 | CD | PRO | B | 384 | 46.524 | −13.377 | 82.121 | 1.00 | 20.49 |
| 3261 | CA | PRO | B | 384 | 47.644 | −13.110 | 79.970 | 1.00 | 24.77 |
| 3262 | CB | PRO | B | 384 | 48.212 | −14.336 | 80.704 | 1.00 | 16.49 |
| 3263 | CG | PRO | B | 384 | 47.919 | −13.981 | 82.165 | 1.00 | 11.88 |
| 3264 | C | PRO | B | 384 | 47.252 | −13.436 | 78.521 | 1.00 | 28.40 |
| 3265 | O | PRO | B | 384 | 46.275 | −14.170 | 78.245 | 1.00 | 35.48 |
| 3266 | N | ASN | B | 385 | 47.998 | −12.840 | 77.607 | 1.00 | 27.81 |
| 3267 | CA | ASN | B | 385 | 47.841 | −13.074 | 76.202 | 1.00 | 25.50 |
| 3268 | CB | ASN | B | 385 | 48.117 | −14.551 | 75.896 | 1.00 | 26.56 |
| 3269 | CG | ASN | B | 385 | 49.579 | −14.914 | 76.084 | 1.00 | 29.73 |
| 3270 | OD1 | ASN | B | 385 | 50.078 | −15.015 | 77.200 | 1.00 | 25.94 |
| 3271 | ND2 | ASN | B | 385 | 50.274 | −15.103 | 74.977 | 1.00 | 42.58 |
| 3272 | C | ASN | B | 385 | 46.559 | −12.664 | 75.558 | 1.00 | 23.58 |
| 3273 | O | ASN | B | 385 | 45.944 | −13.463 | 74.901 | 1.00 | 30.94 |
| 3274 | N | VAL | B | 386 | 46.162 | −11.418 | 75.726 | 1.00 | 22.19 |
| 3275 | CA | VAL | B | 386 | 44.952 | −10.898 | 75.103 | 1.00 | 20.30 |
| 3276 | CB | VAL | B | 386 | 44.150 | −10.087 | 76.141 | 1.00 | 20.63 |
| 3277 | CG1 | VAL | B | 386 | 42.930 | −9.437 | 75.494 | 1.00 | 11.24 |
| 3278 | CG2 | VAL | B | 386 | 43.749 | −11.017 | 77.277 | 1.00 | 13.39 |
| 3279 | C | VAL | B | 386 | 45.393 | −10.003 | 73.923 | 1.00 | 24.43 |
| 3280 | O | VAL | B | 386 | 46.189 | −9.046 | 74.085 | 1.00 | 26.09 |
| 3281 | N | GLN | B | 387 | 44.883 | −10.303 | 72.732 | 1.00 | 26.72 |
| 3282 | CA | GLN | B | 387 | 45.245 | −9.536 | 71.536 | 1.00 | 24.67 |
| 3283 | CB | GLN | B | 387 | 45.170 | −10.437 | 70.315 | 1.00 | 33.85 |
| 3284 | CG | GLN | B | 387 | 46.258 | −11.505 | 70.260 | 1.00 | 48.03 |
| 3285 | CD | GLN | B | 387 | 46.156 | −12.422 | 69.054 | 1.00 | 57.45 |
| 3286 | OE1 | GLN | B | 387 | 46.907 | −13.398 | 68.963 | 1.00 | 62.38 |
| 3287 | NE2 | GLN | B | 387 | 45.233 | −12.118 | 68.119 | 1.00 | 59.65 |
| 3288 | C | GLN | B | 387 | 44.414 | −8.274 | 71.316 | 1.00 | 20.65 |
| 3289 | O | GLN | B | 387 | 44.804 | −7.346 | 70.616 | 1.00 | 20.76 |
| 3290 | N | GLU | B | 388 | 43.260 | −8.212 | 71.930 | 1.00 | 17.86 |
| 3291 | CA | GLU | B | 388 | 42.499 | −7.013 | 71.754 | 1.00 | 16.12 |
| 3292 | CB | GLU | B | 388 | 41.348 | −7.271 | 70.804 | 1.00 | 14.57 |
| 3293 | CG | GLU | B | 388 | 41.835 | −7.575 | 69.398 | 1.00 | 5.36 |
| 3294 | CD | GLU | B | 388 | 40.693 | −7.841 | 68.436 | 1.00 | 12.71 |
| 3295 | OE1 | GLU | B | 388 | 40.072 | −8.916 | 68.529 | 1.00 | 29.33 |
| 3296 | OE2 | GLU | B | 388 | 40.377 | −6.984 | 67.594 | 1.00 | 32.32 |
| 3297 | C | GLU | B | 388 | 42.013 | −6.445 | 73.076 | 1.00 | 11.41 |
| 3298 | O | GLU | B | 388 | 40.808 | −6.341 | 73.320 | 1.00 | 13.14 |
| 3299 | N | PRO | B | 389 | 42.958 | −6.055 | 73.938 | 1.00 | 6.71 |
| 3300 | CD | PRO | B | 389 | 44.404 | −6.004 | 73.676 | 1.00 | 8.43 |
| 3301 | CA | PRO | B | 389 | 42.642 | −5.481 | 75.241 | 1.00 | 14.43 |
| 3302 | CB | PRO | B | 389 | 44.008 | −5.126 | 75.798 | 1.00 | 10.21 |
| 3303 | CG | PRO | B | 389 | 44.809 | −4.909 | 74.567 | 1.00 | 16.14 |
| 3304 | C | PRO | B | 389 | 41.707 | −4.273 | 75.120 | 1.00 | 22.57 |
| 3305 | O | PRO | B | 389 | 40.637 | −4.222 | 75.761 | 1.00 | 27.08 |
| 3306 | N | GLY | B | 390 | 42.089 | −3.298 | 74.299 | 1.00 | 25.43 |
| 3307 | CA | GLY | B | 390 | 41.218 | −2.146 | 74.161 | 1.00 | 25.69 |
| 3308 | C | GLY | B | 390 | 39.768 | −2.583 | 73.961 | 1.00 | 24.27 |
| 3309 | O | GLY | B | 390 | 38.809 | −2.072 | 74.561 | 1.00 | 27.74 |
| 3310 | N | ARG | B | 391 | 39.619 | −3.569 | 73.099 | 1.00 | 21.41 |
| 3311 | CA | ARG | B | 391 | 38.315 | −4.089 | 72.785 | 1.00 | 18.95 |
| 3312 | CB | ARG | B | 391 | 38.449 | −5.049 | 71.614 | 1.00 | 13.78 |
| 3313 | CG | ARG | B | 391 | 37.148 | −5.406 | 71.026 | 1.00 | 23.30 |
| 3314 | CD | ARG | B | 391 | 37.301 | −5.840 | 69.585 | 1.00 | 18.87 |
| 3315 | NE | ARG | B | 391 | 36.124 | −6.604 | 69.192 | 1.00 | 14.58 |
| 3316 | CZ | ARG | B | 391 | 36.188 | −7.622 | 68.359 | 1.00 | 28.26 |
| 3317 | NH1 | ARG | B | 391 | 37.376 | −7.966 | 67.850 | 1.00 | 32.20 |
| 3318 | NH2 | ARG | B | 391 | 35.077 | −8.280 | 68.049 | 1.00 | 40.77 |
| 3319 | C | ARG | B | 391 | 37.680 | −4.759 | 74.004 | 1.00 | 19.06 |
| 3320 | O | ARG | B | 391 | 36.495 | −4.530 | 74.321 | 1.00 | 19.44 |
| 3321 | N | VAL | B | 392 | 38.455 | −5.589 | 74.697 | 1.00 | 16.37 |
| 3322 | CA | VAL | B | 392 | 37.925 | −6.241 | 75.892 | 1.00 | 20.01 |
| 3323 | CB | VAL | B | 392 | 38.947 | −7.292 | 76.450 | 1.00 | 23.55 |
| 3324 | CG1 | VAL | B | 392 | 38.544 | −7.800 | 77.854 | 1.00 | 25.16 |
| 3325 | CG2 | VAL | B | 392 | 38.975 | −8.485 | 75.493 | 1.00 | 22.93 |
| 3326 | C | VAL | B | 392 | 37.569 | −5.165 | 76.960 | 1.00 | 19.85 |
| 3327 | O | VAL | B | 392 | 36.435 | −5.106 | 77.479 | 1.00 | 17.66 |
| 3328 | N | GLU | B | 393 | 38.510 | −4.280 | 77.260 | 1.00 | 19.42 |
| 3329 | CA | GLU | B | 393 | 38.203 | −3.262 | 78.237 | 1.00 | 21.86 |
| 3330 | CB | GLU | B | 393 | 39.319 | −2.215 | 78.329 | 1.00 | 21.31 |
| 3331 | CG | GLU | B | 393 | 39.323 | −1.489 | 79.696 | 1.00 | 15.00 |
| 3332 | CD | GLU | B | 393 | 40.435 | −0.488 | 79.825 | 1.00 | 18.00 |
| 3333 | OE1 | GLU | B | 393 | 41.492 | −0.681 | 79.209 | 1.00 | 36.45 |
| 3334 | OE2 | GLU | B | 393 | 40.274 | 0.510 | 80.541 | 1.00 | 26.03 |
| 3335 | C | GLU | B | 393 | 36.870 | −2.578 | 77.920 | 1.00 | 22.57 |
| 3336 | O | GLU | B | 393 | 36.132 | −2.213 | 78.819 | 1.00 | 25.44 |
| 3337 | N | ALA | B | 394 | 36.531 | −2.424 | 76.651 | 1.00 | 19.40 |
| 3338 | CA | ALA | B | 394 | 35.288 | −1.766 | 76.340 | 1.00 | 14.16 |
| 3339 | CB | ALA | B | 394 | 35.291 | −1.385 | 74.928 | 1.00 | 8.52 |
| 3340 | C | ALA | B | 394 | 34.094 | −2.661 | 76.638 | 1.00 | 17.63 |
| 3341 | O | ALA | B | 394 | 33.086 | −2.197 | 77.154 | 1.00 | 17.76 |
| 3342 | N | LEU | B | 395 | 34.186 | −3.945 | 76.297 | 1.00 | 20.44 |
| 3343 | CA | LEU | B | 395 | 33.070 | −4.870 | 76.557 | 1.00 | 26.34 |
| 3344 | CB | LEU | B | 395 | 33.430 | −6.306 | 76.095 | 1.00 | 28.81 |
| 3345 | CG | LEU | B | 395 | 33.585 | −6.556 | 74.589 | 1.00 | 30.31 |
| 3346 | CD1 | LEU | B | 395 | 34.129 | −7.967 | 74.323 | 1.00 | 19.04 |
| 3347 | CD2 | LEU | B | 395 | 32.248 | −6.358 | 73.917 | 1.00 | 18.75 |
| 3348 | C | LEU | B | 395 | 32.668 | −4.890 | 78.039 | 1.00 | 24.18 |
| 3349 | O | LEU | B | 395 | 31.505 | −4.972 | 78.399 | 1.00 | 24.80 |
| 3350 | N | GLN | B | 396 | 33.660 | −4.785 | 78.898 | 1.00 | 22.89 |
| 3351 | CA | GLN | B | 396 | 33.409 | −4.813 | 80.316 | 1.00 | 23.73 |
| 3352 | CB | GLN | B | 396 | 34.740 | −5.018 | 81.060 | 1.00 | 18.79 |
| 3353 | CG | GLN | B | 396 | 34.581 | −5.032 | 82.527 | 1.00 | 26.79 |
| 3354 | CD | GLN | B | 396 | 35.865 | −5.097 | 83.232 | 1.00 | 27.74 |
| 3355 | OE1 | GLN | B | 396 | 36.681 | −4.191 | 83.117 | 1.00 | 35.13 |
| 3356 | NE2 | GLN | B | 396 | 36.078 | −6.174 | 83.979 | 1.00 | 35.02 |
| 3357 | C | GLN | B | 396 | 32.671 | −3.592 | 80.863 | 1.00 | 23.41 |
| 3358 | O | GLN | B | 396 | 31.845 | −3.714 | 81.759 | 1.00 | 25.74 |
| 3359 | N | GLN | B | 397 | 32.967 | −2.413 | 80.341 | 1.00 | 20.51 |
| 3360 | CA | GLN | B | 397 | 32.300 | −1.202 | 80.833 | 1.00 | 21.67 |
| 3361 | CB | GLN | B | 397 | 32.679 | −0.005 | 79.956 | 1.00 | 14.93 |
| 3362 | CG | GLN | B | 397 | 34.175 | 0.084 | 79.732 | 1.00 | 33.91 |
| 3363 | CD | GLN | B | 397 | 34.957 | 0.475 | 80.982 | 1.00 | 34.76 |
| 3364 | OE1 | GLN | B | 397 | 34.782 | 1.594 | 81.504 | 1.00 | 41.56 |
| 3365 | NE2 | GLN | B | 397 | 35.832 | −0.434 | 81.463 | 1.00 | 24.98 |
| 3366 | C | GLN | B | 397 | 30.748 | −1.288 | 80.956 | 1.00 | 18.34 |
| 3367 | O | GLN | B | 397 | 30.154 | −1.031 | 82.029 | 1.00 | 15.53 |
| 3368 | N | PRO | B | 398 | 30.082 | −1.701 | 79.879 | 1.00 | 17.23 |
| 3369 | CD | PRO | B | 398 | 30.564 | −2.367 | 78.652 | 1.00 | 12.02 |
| 3370 | CA | PRO | B | 398 | 28.624 | −1.757 | 79.990 | 1.00 | 13.09 |
| 3371 | CB | PRO | B | 398 | 28.203 | −2.431 | 78.688 | 1.00 | 13.76 |
| 3372 | CG | PRO | B | 398 | 29.363 | −2.218 | 77.750 | 1.00 | 22.69 |
| 3373 | C | PRO | B | 398 | 28.185 | −2.525 | 81.223 | 1.00 | 13.98 |
| 3374 | O | PRO | B | 398 | 27.182 | −2.197 | 81.859 | 1.00 | 17.80 |
| 3375 | N | TYR | B | 399 | 28.933 | −3.574 | 81.556 | 1.00 | 18.65 |
| 3376 | CA | TYR | B | 399 | 28.615 | −4.414 | 82.725 | 1.00 | 19.43 |
| 3377 | CB | TYR | B | 399 | 29.343 | −5.754 | 82.666 | 1.00 | 11.20 |
| 3378 | CG | TYR | B | 399 | 28.852 | −6.615 | 81.555 | 1.00 | 15.79 |
| 3379 | CD1 | TYR | B | 399 | 27.613 | −7.241 | 81.641 | 1.00 | 12.49 |
| 3380 | CE1 | TYR | B | 399 | 27.121 | −7.990 | 80.583 | 1.00 | 6.49 |
| 3381 | CD2 | TYR | B | 399 | 29.603 | −6.760 | 80.380 | 1.00 | 20.08 |
| 3382 | CE2 | TYR | B | 399 | 29.137 | −7.490 | 79.322 | 1.00 | 7.84 |
| 3383 | CZ | TYR | B | 399 | 27.882 | −8.114 | 79.419 | 1.00 | 19.70 |
| 3384 | OH | TYR | B | 399 | 27.414 | −8.862 | 78.341 | 1.00 | 22.13 |
| 3385 | C | TYR | B | 399 | 28.980 | −3.709 | 84.017 | 1.00 | 18.81 |
| 3386 | O | TYR | B | 399 | 28.216 | −3.749 | 84.967 | 1.00 | 20.59 |
| 3387 | N | VAL | B | 400 | 30.131 | −3.049 | 84.069 | 1.00 | 16.23 |
| 3388 | CA | VAL | B | 400 | 30.460 | −2.334 | 85.297 | 1.00 | 19.14 |
| 3389 | CB | VAL | B | 400 | 31.853 | −1.643 | 85.227 | 1.00 | 18.68 |
| 3390 | CG1 | VAL | B | 400 | 31.994 | −0.551 | 86.289 | 1.00 | 5.30 |
| 3391 | CG2 | VAL | B | 400 | 32.919 | −2.691 | 85.449 | 1.00 | 19.53 |
| 3392 | C | VAL | B | 400 | 29.384 | −1.288 | 85.544 | 1.00 | 17.89 |
| 3393 | O | VAL | B | 400 | 28.964 | −1.110 | 86.664 | 1.00 | 23.07 |
| 3394 | N | GLU | B | 401 | 28.911 | −0.623 | 84.495 | 1.00 | 15.04 |
| 3395 | CA | GLU | B | 401 | 27.886 | 0.398 | 84.662 | 1.00 | 11.15 |
| 3396 | CB | GLU | B | 401 | 27.698 | 1.140 | 83.360 | 1.00 | 7.42 |
| 3397 | CG | GLU | B | 401 | 26.608 | 2.123 | 83.364 | 1.00 | 20.38 |
| 3398 | CD | GLU | B | 401 | 26.757 | 3.074 | 82.228 | 1.00 | 37.37 |
| 3399 | OE1 | GLU | B | 401 | 26.867 | 2.620 | 81.057 | 1.00 | 39.44 |
| 3400 | OE2 | GLU | B | 401 | 26.780 | 4.282 | 82.518 | 1.00 | 39.71 |
| 3401 | C | GLU | B | 401 | 26.557 | −0.211 | 85.119 | 1.00 | 13.41 |
| 3402 | O | GLU | B | 401 | 25.847 | 0.386 | 85.938 | 1.00 | 12.31 |
| 3403 | N | ALA | B | 402 | 26.225 | −1.404 | 84.623 | 1.00 | 11.22 |
| 3404 | CA | ALA | B | 402 | 24.965 | −2.000 | 85.000 | 1.00 | 12.10 |
| 3405 | CB | ALA | B | 402 | 24.760 | −3.240 | 84.180 | 1.00 | 15.56 |
| 3406 | C | ALA | B | 402 | 25.021 | −2.304 | 86.519 | 1.00 | 16.14 |
| 3407 | O | ALA | B | 402 | 24.032 | −2.230 | 87.233 | 1.00 | 16.28 |
| 3408 | N | LEU | B | 403 | 26.211 | −2.608 | 87.018 | 1.00 | 14.33 |
| 3409 | CA | LEU | B | 403 | 26.364 | −2.917 | 88.424 | 1.00 | 12.70 |
| 3410 | CB | LEU | B | 403 | 27.694 | −3.571 | 88.660 | 1.00 | 14.10 |
| 3411 | CG | LEU | B | 403 | 27.950 | −4.153 | 90.036 | 1.00 | 5.25 |
| 3412 | CD1 | LEU | B | 403 | 27.039 | −5.370 | 90.321 | 1.00 | 5.25 |
| 3413 | CD2 | LEU | B | 403 | 29.407 | −4.531 | 90.070 | 1.00 | 13.26 |
| 3414 | C | LEU | B | 403 | 26.279 | −1.670 | 89.270 | 1.00 | 16.33 |
| 3415 | O | LEU | B | 403 | 25.606 | −1.654 | 90.309 | 1.00 | 20.44 |
| 3416 | N | LEU | B | 404 | 26.979 | −0.630 | 88.825 | 1.00 | 15.15 |
| 3417 | CA | LEU | B | 404 | 26.984 | 0.646 | 89.500 | 1.00 | 18.81 |
| 3418 | CB | LEU | B | 404 | 27.784 | 1.664 | 88.701 | 1.00 | 9.81 |
| 3419 | CG | LEU | B | 404 | 27.772 | 3.017 | 89.442 | 1.00 | 19.99 |
| 3420 | CD1 | LEU | B | 404 | 28.565 | 2.881 | 90.769 | 1.00 | 21.31 |
| 3421 | CD2 | LEU | B | 404 | 28.367 | 4.094 | 88.591 | 1.00 | 17.42 |
| 3422 | C | LEU | B | 404 | 25.567 | 1.200 | 89.713 | 1.00 | 25.26 |
| 3423 | O | LEU | B | 404 | 25.214 | 1.654 | 90.819 | 1.00 | 27.46 |
| 3424 | N | SER | B | 405 | 24.770 | 1.173 | 88.645 | 1.00 | 28.43 |
| 3425 | CA | SER | B | 405 | 23.413 | 1.681 | 88.708 | 1.00 | 30.86 |
| 3426 | CB | SER | B | 405 | 22.834 | 1.806 | 87.291 | 1.00 | 27.87 |
| 3427 | OG | SER | B | 405 | 21.426 | 1.931 | 87.341 | 1.00 | 49.84 |
| 3428 | C | SER | B | 405 | 22.572 | 0.760 | 89.589 | 1.00 | 28.69 |
| 3429 | O | SER | B | 405 | 21.738 | 1.217 | 90.362 | 1.00 | 34.53 |
| 3430 | N | TYR | B | 406 | 22.827 | −0.536 | 89.498 | 1.00 | 25.87 |
| 3431 | CA | TYR | B | 406 | 22.091 | −1.514 | 90.290 | 1.00 | 23.94 |
| 3432 | CB | TYR | B | 406 | 22.499 | −2.918 | 89.855 | 1.00 | 16.25 |
| 3433 | CG | TYR | B | 406 | 21.671 | −3.995 | 90.459 | 1.00 | 22.54 |
| 3434 | CD1 | TYR | B | 406 | 20.454 | −4.365 | 89.886 | 1.00 | 24.85 |
| 3435 | CE1 | TYR | B | 406 | 19.646 | −5.363 | 90.474 | 1.00 | 24.09 |
| 3436 | CD2 | TYR | B | 406 | 22.082 | −4.640 | 91.638 | 1.00 | 27.13 |
| 3437 | CE2 | TYR | B | 406 | 21.291 | −5.633 | 92.237 | 1.00 | 26.94 |
| 3438 | CZ | TYR | B | 406 | 20.065 | −5.997 | 91.643 | 1.00 | 30.69 |
| 3439 | OH | TYR | B | 406 | 19.290 | −7.027 | 92.192 | 1.00 | 26.76 |
| 3440 | C | TYR | B | 406 | 22.328 | −1.357 | 91.802 | 1.00 | 22.98 |
| 3441 | O | TYR | B | 406 | 21.387 | −1.179 | 92.575 | 1.00 | 22.41 |
| 3442 | N | THR | B | 407 | 23.588 | −1.408 | 92.221 | 1.00 | 21.64 |
| 3443 | CA | THR | B | 407 | 23.910 | −1.304 | 93.641 | 1.00 | 22.30 |
| 3444 | CB | THR | B | 407 | 25.424 | −1.403 | 93.879 | 1.00 | 18.93 |
| 3445 | OG1 | THR | B | 407 | 26.077 | −0.330 | 93.181 | 1.00 | 20.67 |
| 3446 | CG2 | THR | B | 407 | 25.967 | −2.755 | 93.419 | 1.00 | 10.74 |
| 3447 | C | THR | B | 407 | 23.441 | 0.027 | 94.224 | 1.00 | 27.40 |
| 3448 | O | THR | B | 407 | 23.142 | 0.125 | 95.438 | 1.00 | 28.52 |
| 3449 | N | ARG | B | 408 | 23.366 | 1.043 | 93.359 | 1.00 | 25.32 |
| 3450 | CA | ARG | B | 408 | 22.941 | 2.364 | 93.778 | 1.00 | 22.43 |
| 3451 | CB | ARG | B | 408 | 23.170 | 3.363 | 92.651 | 1.00 | 18.62 |
| 3452 | CG | ARG | B | 408 | 23.166 | 4.801 | 93.095 | 1.00 | 31.32 |
| 3453 | CD | ARG | B | 408 | 24.367 | 5.146 | 93.954 | 1.00 | 39.94 |
| 3454 | NE | ARG | B | 408 | 24.314 | 6.530 | 94.424 | 1.00 | 62.46 |
| 3455 | CZ | ARG | B | 408 | 24.457 | 7.599 | 93.640 | 1.00 | 70.70 |
| 3456 | NH1 | ARG | B | 408 | 24.671 | 7.436 | 92.342 | 1.00 | 71.99 |
| 3457 | NH2 | ARG | B | 408 | 24.363 | 8.830 | 94.146 | 1.00 | 68.86 |
| 3458 | C | ARG | B | 408 | 21.463 | 2.331 | 94.153 | 1.00 | 25.92 |
| 3459 | O | ARG | B | 408 | 21.051 | 2.960 | 95.132 | 1.00 | 29.04 |
| 3460 | N | ILE | B | 409 | 20.671 | 1.594 | 93.380 | 1.00 | 23.14 |
| 3461 | CA | ILE | B | 409 | 19.245 | 1.477 | 93.624 | 1.00 | 24.07 |
| 3462 | CB | ILE | B | 409 | 18.549 | 0.982 | 92.334 | 1.00 | 31.63 |
| 3463 | CG2 | ILE | B | 409 | 17.158 | 0.415 | 92.611 | 1.00 | 31.53 |
| 3464 | CG1 | ILE | B | 409 | 18.472 | 2.131 | 91.336 | 1.00 | 27.41 |
| 3465 | CD1 | ILE | B | 409 | 18.056 | 1.651 | 89.934 | 1.00 | 38.63 |
| 3466 | C | ILE | B | 409 | 18.958 | 0.517 | 94.781 | 1.00 | 29.16 |
| 3467 | O | ILE | B | 409 | 17.977 | 0.679 | 95.503 | 1.00 | 30.54 |
| 3468 | N | LYS | B | 410 | 19.820 | −0.481 | 94.951 | 1.00 | 30.14 |
| 3469 | CA | LYS | B | 410 | 19.672 | −1.457 | 96.019 | 1.00 | 29.72 |
| 3470 | CB | LYS | B | 410 | 20.548 | −2.682 | 95.741 | 1.00 | 28.57 |
| 3471 | CG | LYS | B | 410 | 20.378 | −3.815 | 96.754 | 1.00 | 34.90 |
| 3472 | CD | LYS | B | 410 | 21.126 | −5.069 | 96.330 | 1.00 | 34.66 |
| 3473 | CE | LYS | B | 410 | 20.786 | −6.234 | 97.205 | 1.00 | 22.65 |
| 3474 | NZ | LYS | B | 410 | 21.487 | −7.471 | 96.748 | 1.00 | 27.10 |
| 3475 | C | LYS | B | 410 | 20.048 | −0.861 | 97.389 | 1.00 | 27.38 |
| 3476 | O | LYS | B | 410 | 19.389 | −1.134 | 98.380 | 1.00 | 28.37 |
| 3477 | N | ARG | B | 411 | 21.101 | −0.060 | 97.467 | 1.00 | 23.84 |
| 3478 | CA | ARG | B | 411 | 21.446 | 0.510 | 98.773 | 1.00 | 24.63 |
| 3479 | CB | ARG | B | 411 | 22.529 | −0.333 | 99.478 | 1.00 | 26.88 |
| 3480 | CG | ARG | B | 411 | 22.214 | −1.822 | 99.449 | 1.00 | 31.02 |
| 3481 | CD | ARG | B | 411 | 22.225 | −2.418 | 100.835 | 1.00 | 40.94 |
| 3482 | NE | ARG | B | 411 | 23.438 | −3.184 | 101.133 | 1.00 | 44.64 |
| 3483 | CZ | ARG | B | 411 | 23.475 | −4.259 | 101.923 | 1.00 | 39.45 |
| 3484 | NH1 | ARG | B | 411 | 22.363 | −4.700 | 102.486 | 1.00 | 49.81 |
| 3485 | NH2 | ARG | B | 411 | 24.625 | −4.880 | 102.173 | 1.00 | 37.84 |
| 3486 | C | ARG | B | 411 | 21.894 | 1.942 | 98.541 | 1.00 | 20.38 |
| 3487 | O | ARG | B | 411 | 23.078 | 2.252 | 98.482 | 1.00 | 26.11 |
| 3488 | N | PRO | B | 412 | 20.918 | 2.841 | 98.398 | 1.00 | 17.18 |
| 3489 | CD | PRO | B | 412 | 19.491 | 2.471 | 98.413 | 1.00 | 10.00 |
| 3490 | CA | PRO | B | 412 | 21.106 | 4.270 | 98.143 | 1.00 | 11.86 |
| 3491 | CB | PRO | B | 412 | 19.703 | 4.801 | 98.122 | 1.00 | 5.25 |
| 3492 | CG | PRO | B | 412 | 18.885 | 3.585 | 97.700 | 1.00 | 6.95 |
| 3493 | C | PRO | B | 412 | 21.933 | 4.941 | 99.151 | 1.00 | 17.42 |
| 3494 | O | PRO | B | 412 | 22.670 | 5.826 | 98.814 | 1.00 | 15.03 |
| 3495 | N | GLN | B | 413 | 21.808 | 4.499 | 100.400 | 1.00 | 27.59 |
| 3496 | CA | GLN | B | 413 | 22.542 | 5.104 | 101.502 | 1.00 | 28.72 |
| 3497 | CB | GLN | B | 413 | 21.660 | 5.093 | 102.749 | 1.00 | 40.02 |
| 3498 | CG | GLN | B | 413 | 20.436 | 6.039 | 102.688 | 1.00 | 58.84 |
| 3499 | CD | GLN | B | 413 | 19.102 | 5.279 | 102.647 | 1.00 | 73.73 |
| 3500 | OE1 | GLN | B | 413 | 18.725 | 4.708 | 101.606 | 1.00 | 77.93 |
| 3501 | NE2 | GLN | B | 413 | 18.398 | 5.240 | 103.789 | 1.00 | 69.98 |
| 3502 | C | GLN | B | 413 | 23.896 | 4.451 | 101.802 | 1.00 | 29.82 |
| 3503 | O | GLN | B | 413 | 24.511 | 4.711 | 102.836 | 1.00 | 37.66 |
| 3504 | N | ASP | B | 414 | 24.365 | 3.594 | 100.905 | 1.00 | 26.65 |
| 3505 | CA | ASP | B | 414 | 25.645 | 2.928 | 101.086 | 1.00 | 19.28 |
| 3506 | CB | ASP | B | 414 | 25.455 | 1.499 | 101.596 | 1.00 | 17.43 |
| 3507 | CG | ASP | B | 414 | 26.779 | 0.815 | 101.943 | 1.00 | 18.73 |
| 3508 | OD1 | ASP | B | 414 | 27.805 | 1.519 | 102.103 | 1.00 | 26.26 |
| 3509 | OD2 | ASP | B | 414 | 26.785 | −0.421 | 102.075 | 1.00 | 6.65 |
| 3510 | C | ASP | B | 414 | 26.393 | 2.906 | 99.763 | 1.00 | 17.97 |
| 3511 | O | ASP | B | 414 | 26.588 | 1.853 | 99.171 | 1.00 | 13.81 |
| 3512 | N | GLN | B | 415 | 26.831 | 4.079 | 99.324 | 1.00 | 18.40 |
| 3513 | CA | GLN | B | 415 | 27.526 | 4.162 | 98.079 | 1.00 | 23.60 |
| 3514 | CB | GLN | B | 415 | 27.506 | 5.585 | 97.547 | 1.00 | 30.14 |
| 3515 | CG | GLN | B | 415 | 28.044 | 6.586 | 98.515 | 1.00 | 47.25 |
| 3516 | CD | GLN | B | 415 | 28.254 | 7.923 | 97.837 | 1.00 | 59.35 |
| 3517 | OE1 | GLN | B | 415 | 27.408 | 8.350 | 97.021 | 1.00 | 57.51 |
| 3518 | NE2 | GLN | B | 415 | 29.378 | 8.599 | 98.160 | 1.00 | 51.01 |
| 3519 | C | GLN | B | 415 | 28.941 | 3.629 | 98.135 | 1.00 | 22.02 |
| 3520 | O | GLN | B | 415 | 29.747 | 3.860 | 97.217 | 1.00 | 23.65 |
| 3521 | N | LEU | B | 416 | 29.265 | 2.914 | 99.200 | 1.00 | 19.34 |
| 3522 | CA | LEU | B | 416 | 30.599 | 2.325 | 99.251 | 1.00 | 20.84 |
| 3523 | CB | LEU | B | 416 | 31.280 | 2.641 | 100.572 | 1.00 | 19.15 |
| 3524 | CG | LEU | B | 416 | 31.886 | 4.041 | 100.799 | 1.00 | 25.91 |
| 3525 | CD1 | LEU | B | 416 | 33.230 | 3.855 | 101.446 | 1.00 | 24.35 |
| 3526 | CD2 | LEU | B | 416 | 32.101 | 4.795 | 99.502 | 1.00 | 24.30 |
| 3527 | C | LEU | B | 416 | 30.525 | 0.795 | 99.020 | 1.00 | 21.13 |
| 3528 | O | LEU | B | 416 | 31.530 | 0.097 | 98.900 | 1.00 | 17.19 |
| 3529 | N | ARG | B | 417 | 29.304 | 0.284 | 98.930 | 1.00 | 24.16 |
| 3530 | CA | ARG | B | 417 | 29.069 | −1.136 | 98.685 | 1.00 | 21.61 |
| 3531 | CB | ARG | B | 417 | 27.565 | −1.406 | 98.680 | 1.00 | 29.84 |
| 3532 | CG | ARG | B | 417 | 27.165 | −2.819 | 98.393 | 1.00 | 16.14 |
| 3533 | CD | ARG | B | 417 | 25.781 | −3.094 | 98.956 | 1.00 | 17.27 |
| 3534 | NE | ARG | B | 417 | 25.517 | −4.520 | 98.917 | 1.00 | 19.36 |
| 3535 | CZ | ARG | B | 417 | 24.965 | −5.158 | 97.897 | 1.00 | 21.88 |
| 3536 | NH1 | ARG | B | 417 | 24.567 | −4.531 | 96.799 | 1.00 | 24.73 |
| 3537 | NH2 | ARG | B | 417 | 24.871 | −6.461 | 97.962 | 1.00 | 26.24 |
| 3538 | C | ARG | B | 417 | 29.639 | −1.467 | 97.316 | 1.00 | 22.39 |
| 3539 | O | ARG | B | 417 | 30.498 | −2.350 | 97.178 | 1.00 | 19.83 |
| 3540 | N | PHE | B | 418 | 29.171 | −0.757 | 96.288 | 1.00 | 21.46 |
| 3541 | CA | PHE | B | 418 | 29.688 | −1.037 | 94.946 | 1.00 | 19.87 |
| 3542 | CB | PHE | B | 418 | 29.267 | 0.028 | 93.977 | 1.00 | 13.06 |
| 3543 | CG | PHE | B | 418 | 29.834 | −0.152 | 92.643 | 1.00 | 17.77 |
| 3544 | CD1 | PHE | B | 418 | 29.412 | −1.212 | 91.841 | 1.00 | 27.33 |
| 3545 | CD2 | PHE | B | 418 | 30.830 | 0.700 | 92.186 | 1.00 | 19.47 |
| 3546 | CE1 | PHE | B | 418 | 29.986 | −1.432 | 90.593 | 1.00 | 22.32 |
| 3547 | CE2 | PHE | B | 418 | 31.420 | 0.488 | 90.927 | 1.00 | 19.06 |
| 3548 | CZ | PHE | B | 418 | 30.997 | −0.571 | 90.144 | 1.00 | 27.55 |
| 3549 | C | PHE | B | 418 | 31.220 | −1.189 | 94.909 | 1.00 | 18.37 |
| 3550 | O | PHE | B | 418 | 31.737 | −2.168 | 94.371 | 1.00 | 20.12 |
| 3551 | N | PRO | B | 419 | 31.958 | −0.242 | 95.505 | 1.00 | 14.48 |
| 3552 | CD | PRO | B | 419 | 31.567 | 1.103 | 95.939 | 1.00 | 12.36 |
| 3553 | CA | PRO | B | 419 | 33.410 | −0.359 | 95.494 | 1.00 | 14.31 |
| 3554 | CB | PRO | B | 419 | 33.863 | 0.945 | 96.141 | 1.00 | 15.64 |
| 3555 | CG | PRO | B | 419 | 32.884 | 1.896 | 95.745 | 1.00 | 5.25 |
| 3556 | C | PRO | B | 419 | 33.894 | −1.589 | 96.264 | 1.00 | 18.29 |
| 3557 | O | PRO | B | 419 | 34.961 | −2.131 | 95.950 | 1.00 | 22.41 |
| 3558 | N | ARG | B | 420 | 33.131 | −2.039 | 97.262 | 1.00 | 17.55 |
| 3559 | CA | ARG | B | 420 | 33.563 | −3.218 | 98.030 | 1.00 | 18.76 |
| 3560 | CB | ARG | B | 420 | 32.657 | −3.515 | 99.228 | 1.00 | 16.09 |
| 3561 | CG | ARG | B | 420 | 32.818 | −2.592 | 100.389 | 1.00 | 23.27 |
| 3562 | CD | ARG | B | 420 | 32.034 | −3.061 | 101.593 | 1.00 | 31.13 |
| 3563 | NE | ARG | B | 420 | 32.051 | −2.010 | 102.608 | 1.00 | 53.34 |
| 3564 | CZ | ARG | B | 420 | 31.211 | −0.972 | 102.659 | 1.00 | 53.62 |
| 3565 | NH1 | ARG | B | 420 | 30.249 | −0.838 | 101.764 | 1.00 | 45.38 |
| 3566 | NH2 | ARG | B | 420 | 31.366 | −0.038 | 103.594 | 1.00 | 56.87 |
| 3567 | C | ARG | B | 420 | 33.516 | −4.405 | 97.092 | 1.00 | 23.08 |
| 3568 | O | ARG | B | 420 | 34.392 | −5.272 | 97.113 | 1.00 | 25.02 |
| 3569 | N | MET | B | 421 | 32.473 | −4.471 | 96.271 | 1.00 | 26.24 |
| 3570 | CA | MET | B | 421 | 32.392 | −5.571 | 95.320 | 1.00 | 26.72 |
| 3571 | CB | MET | B | 421 | 31.156 | −5.438 | 94.462 | 1.00 | 26.46 |
| 3572 | CG | MET | B | 421 | 29.952 | −5.847 | 95.217 | 1.00 | 24.52 |
| 3573 | SD | MET | B | 421 | 28.542 | −5.881 | 94.219 | 1.00 | 22.16 |
| 3574 | CE | MET | B | 421 | 27.317 | −5.174 | 95.426 | 1.00 | 22.65 |
| 3575 | C | MET | B | 421 | 33.620 | −5.604 | 94.438 | 1.00 | 26.26 |
| 3576 | O | MET | B | 421 | 34.280 | −6.652 | 94.375 | 1.00 | 31.69 |
| 3577 | N | LEU | B | 422 | 33.927 | −4.465 | 93.791 | 1.00 | 20.52 |
| 3578 | CA | LEU | B | 422 | 35.067 | −4.356 | 92.919 | 1.00 | 15.96 |
| 3579 | CB | LEU | B | 422 | 35.222 | −2.931 | 92.432 | 1.00 | 23.55 |
| 3580 | CG | LEU | B | 422 | 34.273 | −2.525 | 91.276 | 1.00 | 25.39 |
| 3581 | CD1 | LEU | B | 422 | 34.433 | −1.038 | 90.897 | 1.00 | 23.98 |
| 3582 | CD2 | LEU | B | 422 | 34.584 | −3.354 | 90.115 | 1.00 | 26.78 |
| 3583 | C | LEU | B | 422 | 36.295 | −4.815 | 93.652 | 1.00 | 17.24 |
| 3584 | O | LEU | B | 422 | 37.150 | −5.462 | 93.075 | 1.00 | 15.66 |
| 3585 | N | MET | B | 423 | 36.380 | −4.521 | 94.940 | 1.00 | 16.96 |
| 3586 | CA | MET | B | 423 | 37.537 | −4.971 | 95.701 | 1.00 | 18.89 |
| 3587 | CB | MET | B | 423 | 37.480 | −4.349 | 97.097 | 1.00 | 21.05 |
| 3588 | CG | MET | B | 423 | 38.772 | −3.653 | 97.532 | 1.00 | 37.96 |
| 3589 | SD | MET | B | 423 | 39.133 | −2.155 | 96.569 | 1.00 | 48.80 |
| 3590 | CE | MET | B | 423 | 37.726 | −1.154 | 97.116 | 1.00 | 55.61 |
| 3591 | C | MET | B | 423 | 37.628 | −6.540 | 95.775 | 1.00 | 19.54 |
| 3592 | O | MET | B | 423 | 38.704 | −7.109 | 96.010 | 1.00 | 22.90 |
| 3593 | N | LYS | B | 424 | 36.511 | −7.242 | 95.562 | 1.00 | 19.78 |
| 3594 | CA | LYS | B | 424 | 36.522 | −8.709 | 95.587 | 1.00 | 18.28 |
| 3595 | CB | LYS | B | 424 | 35.102 | −9.217 | 95.740 | 1.00 | 14.35 |
| 3596 | CG | LYS | B | 424 | 34.494 | −8.824 | 97.077 | 1.00 | 19.78 |
| 3597 | CD | LYS | B | 424 | 35.192 | −9.579 | 98.220 | 1.00 | 16.47 |
| 3598 | CE | LYS | B | 424 | 34.653 | −9.192 | 99.592 | 1.00 | 26.47 |
| 3599 | NZ | LYS | B | 424 | 35.607 | −8.267 | 100.347 | 1.00 | 29.05 |
| 3600 | C | LYS | B | 424 | 37.192 | −9.278 | 94.314 | 1.00 | 19.45 |
| 3601 | O | LYS | B | 424 | 37.642 | −10.432 | 94.275 | 1.00 | 14.12 |
| 3602 | N | LEU | B | 425 | 37.257 | −8.465 | 93.261 | 1.00 | 20.50 |
| 3603 | CA | LEU | B | 425 | 37.945 | −8.906 | 92.057 | 1.00 | 17.30 |
| 3604 | CB | LEU | B | 425 | 37.693 | −7.970 | 90.891 | 1.00 | 10.42 |
| 3605 | CG | LEU | B | 425 | 36.256 | −7.932 | 90.363 | 1.00 | 22.59 |
| 3606 | CD1 | LEU | B | 425 | 36.129 | −6.841 | 89.275 | 1.00 | 12.90 |
| 3607 | CD2 | LEU | B | 425 | 35.887 | −9.270 | 89.785 | 1.00 | 12.37 |
| 3608 | C | LEU | B | 425 | 39.429 | −8.884 | 92.424 | 1.00 | 15.79 |
| 3609 | O | LEU | B | 425 | 40.200 | −9.738 | 91.989 | 1.00 | 21.26 |
| 3610 | N | VAL | B | 426 | 39.830 | −7.929 | 93.248 | 1.00 | 10.39 |
| 3611 | CA | VAL | B | 426 | 41.229 | −7.853 | 93.642 | 1.00 | 15.67 |
| 3612 | CB | VAL | B | 426 | 41.456 | −6.711 | 94.680 | 1.00 | 15.21 |
| 3613 | CG1 | VAL | B | 426 | 42.861 | −6.798 | 95.251 | 1.00 | 6.06 |
| 3614 | CG2 | VAL | B | 426 | 41.235 | −5.329 | 94.031 | 1.00 | 12.87 |
| 3615 | C | VAL | B | 426 | 41.655 | −9.213 | 94.264 | 1.00 | 19.24 |
| 3616 | O | VAL | B | 426 | 42.706 | −9.820 | 93.930 | 1.00 | 19.27 |
| 3617 | N | SER | B | 427 | 40.823 | −9.702 | 95.166 | 1.00 | 16.66 |
| 3618 | CA | SER | B | 427 | 41.103 | −10.963 | 95.825 | 1.00 | 17.77 |
| 3619 | CB | SER | B | 427 | 40.050 | −11.220 | 96.864 | 1.00 | 15.50 |
| 3620 | OG | SER | B | 427 | 39.982 | −10.130 | 97.760 | 1.00 | 28.60 |
| 3621 | C | SER | B | 427 | 41.148 | −12.136 | 94.875 | 1.00 | 21.81 |
| 3622 | O | SER | B | 427 | 42.012 | −12.995 | 95.008 | 1.00 | 25.86 |
| 3623 | N | LEU | B | 428 | 40.220 | −12.194 | 93.926 | 1.00 | 19.86 |
| 3624 | CA | LEU | B | 428 | 40.257 | −13.302 | 92.987 | 1.00 | 18.69 |
| 3625 | CB | LEU | B | 428 | 39.150 | −13.169 | 91.930 | 1.00 | 21.55 |
| 3626 | CG | LEU | B | 428 | 37.756 | −13.238 | 92.528 | 1.00 | 14.78 |
| 3627 | CD1 | LEU | B | 428 | 36.745 | −12.976 | 91.449 | 1.00 | 13.20 |
| 3628 | CD2 | LEU | B | 428 | 37.579 | −14.607 | 93.183 | 1.00 | 5.25 |
| 3629 | C | LEU | B | 428 | 41.621 | −13.379 | 92.300 | 1.00 | 16.34 |
| 3630 | O | LEU | B | 428 | 42.053 | −14.454 | 91.922 | 1.00 | 20.34 |
| 3631 | N | ARG | B | 429 | 42.292 | −12.250 | 92.118 | 1.00 | 13.61 |
| 3632 | CA | ARG | B | 429 | 43.575 | −12.319 | 91.481 | 1.00 | 14.98 |
| 3633 | CB | ARG | B | 429 | 44.131 | −10.936 | 91.220 | 1.00 | 22.87 |
| 3634 | CG | ARG | B | 429 | 43.547 | −10.244 | 90.010 | 1.00 | 17.78 |
| 3635 | CD | ARG | B | 429 | 43.500 | −11.204 | 88.851 | 1.00 | 26.45 |
| 3636 | NE | ARG | B | 429 | 42.578 | −10.691 | 87.848 | 1.00 | 25.15 |
| 3637 | CZ | ARG | B | 429 | 42.874 | −9.711 | 87.008 | 1.00 | 21.97 |
| 3638 | NH1 | ARG | B | 429 | 44.065 | −9.153 | 87.042 | 1.00 | 19.57 |
| 3639 | NH2 | ARG | B | 429 | 41.969 | −9.278 | 86.147 | 1.00 | 35.54 |
| 3640 | C | ARG | B | 429 | 44.481 | −13.081 | 92.421 | 1.00 | 16.08 |
| 3641 | O | ARG | B | 429 | 45.044 | −14.111 | 92.057 | 1.00 | 21.79 |
| 3642 | N | THR | B | 430 | 44.621 | −12.595 | 93.642 | 1.00 | 14.07 |
| 3643 | CA | THR | B | 430 | 45.452 | −13.296 | 94.620 | 1.00 | 15.97 |
| 3644 | CB | THR | B | 430 | 45.339 | −12.646 | 96.079 | 1.00 | 9.39 |
| 3645 | OG1 | THR | B | 430 | 45.995 | −11.357 | 96.080 | 1.00 | 14.87 |
| 3646 | CG2 | THR | B | 430 | 46.033 | −13.520 | 97.122 | 1.00 | 5.25 |
| 3647 | C | THR | B | 430 | 45.060 | −14.795 | 94.676 | 1.00 | 17.85 |
| 3648 | O | THR | B | 430 | 45.911 | −15.685 | 94.529 | 1.00 | 18.15 |
| 3649 | N | LEU | B | 431 | 43.781 | −15.085 | 94.887 | 1.00 | 17.45 |
| 3650 | CA | LEU | B | 431 | 43.366 | −16.479 | 94.964 | 1.00 | 16.00 |
| 3651 | CB | LEU | B | 431 | 41.852 | −16.597 | 95.030 | 1.00 | 14.31 |
| 3652 | CG | LEU | B | 431 | 41.172 | −16.422 | 96.386 | 1.00 | 10.47 |
| 3653 | CD1 | LEU | B | 431 | 39.683 | −16.470 | 96.162 | 1.00 | 21.26 |
| 3654 | CD2 | LEU | B | 431 | 41.612 | −17.516 | 97.342 | 1.00 | 16.24 |
| 3655 | C | LEU | B | 431 | 43.891 | −17.251 | 93.773 | 1.00 | 16.84 |
| 3656 | O | LEU | B | 431 | 44.572 | −18.284 | 93.947 | 1.00 | 19.07 |
| 3657 | N | SER | B | 432 | 43.593 | −16.736 | 92.575 | 1.00 | 13.15 |
| 3658 | CA | SER | B | 432 | 44.051 | −17.351 | 91.331 | 1.00 | 12.49 |
| 3659 | CB | SER | B | 432 | 43.841 | −16.400 | 90.163 | 1.00 | 5.25 |
| 3660 | OG | SER | B | 432 | 44.497 | −16.878 | 89.014 | 1.00 | 16.69 |
| 3661 | C | SER | B | 432 | 45.518 | −17.704 | 91.466 | 1.00 | 12.52 |
| 3662 | O | SER | B | 432 | 45.979 | −18.771 | 91.060 | 1.00 | 14.34 |
| 3663 | N | SER | B | 433 | 46.248 | −16.811 | 92.102 | 1.00 | 12.12 |
| 3664 | CA | SER | B | 433 | 47.669 | −17.032 | 92.326 | 1.00 | 11.06 |
| 3665 | CB | SER | B | 433 | 48.306 | −15.754 | 92.899 | 1.00 | 7.83 |
| 3666 | OG | SER | B | 433 | 49.703 | −15.834 | 92.934 | 1.00 | 20.00 |
| 3667 | C | SER | B | 433 | 47.909 | −18.200 | 93.265 | 1.00 | 11.27 |
| 3668 | O | SER | B | 433 | 48.640 | −19.084 | 92.941 | 1.00 | 17.43 |
| 3669 | N | VAL | B | 434 | 47.284 | −18.201 | 94.432 | 1.00 | 13.16 |
| 3670 | CA | VAL | B | 434 | 47.480 | −19.271 | 95.410 | 1.00 | 15.82 |
| 3671 | CB | VAL | B | 434 | 46.644 | −19.041 | 96.638 | 1.00 | 13.23 |
| 3672 | CG1 | VAL | B | 434 | 46.769 | −20.194 | 97.581 | 1.00 | 28.07 |
| 3673 | CG2 | VAL | B | 434 | 47.099 | −17.807 | 97.299 | 1.00 | 19.18 |
| 3674 | C | VAL | B | 434 | 47.074 | −20.602 | 94.821 | 1.00 | 17.34 |
| 3675 | O | VAL | B | 434 | 47.662 | −21.655 | 95.136 | 1.00 | 15.32 |
| 3676 | N | HIS | B | 435 | 46.067 | −20.539 | 93.955 | 1.00 | 19.92 |
| 3677 | CA | HIS | B | 435 | 45.558 | −21.719 | 93.263 | 1.00 | 20.76 |
| 3678 | CB | HIS | B | 435 | 44.291 | −21.354 | 92.522 | 1.00 | 16.13 |
| 3679 | CG | HIS | B | 435 | 43.938 | −22.327 | 91.449 | 1.00 | 13.43 |
| 3680 | CD2 | HIS | B | 435 | 44.084 | −22.273 | 90.099 | 1.00 | 19.51 |
| 3681 | ND1 | HIS | B | 435 | 43.386 | −23.552 | 91.723 | 1.00 | 11.55 |
| 3682 | CE1 | HIS | B | 435 | 43.199 | −24.214 | 90.590 | 1.00 | 21.95 |
| 3683 | NE2 | HIS | B | 435 | 43.617 | −23.458 | 89.589 | 1.00 | 18.37 |
| 3684 | C | HIS | B | 435 | 46.587 | −22.325 | 92.265 | 1.00 | 22.94 |
| 3685 | O | HIS | B | 435 | 46.665 | −23.554 | 92.081 | 1.00 | 22.54 |
| 3686 | N | SER | B | 436 | 47.372 | −21.469 | 91.617 | 1.00 | 22.22 |
| 3687 | CA | SER | B | 436 | 48.376 | −21.988 | 90.703 | 1.00 | 26.24 |
| 3688 | CB | SER | B | 436 | 48.882 | −20.900 | 89.784 | 1.00 | 16.08 |
| 3689 | OG | SER | B | 436 | 49.629 | −19.982 | 90.515 | 1.00 | 31.23 |
| 3690 | C | SER | B | 436 | 49.517 | −22.559 | 91.551 | 1.00 | 27.57 |
| 3691 | O | SER | B | 436 | 50.224 | −23.486 | 91.135 | 1.00 | 31.33 |
| 3692 | N | GLU | B | 437 | 49.690 | −22.026 | 92.755 | 1.00 | 24.78 |
| 3693 | CA | GLU | B | 437 | 50.717 | −22.575 | 93.595 | 1.00 | 30.51 |
| 3694 | CB | GLU | B | 437 | 51.020 | −21.669 | 94.765 | 1.00 | 32.25 |
| 3695 | CG | GLU | B | 437 | 51.777 | −20.430 | 94.342 | 1.00 | 52.73 |
| 3696 | CD | GLU | B | 437 | 52.395 | −19.681 | 95.513 | 1.00 | 57.67 |
| 3697 | OE1 | GLU | B | 437 | 51.659 | −18.973 | 96.260 | 1.00 | 58.28 |
| 3698 | OE2 | GLU | B | 437 | 53.627 | −19.813 | 95.688 | 1.00 | 55.84 |
| 3699 | C | GLU | B | 437 | 50.239 | −23.904 | 94.088 | 1.00 | 30.02 |
| 3700 | O | GLU | B | 437 | 51.023 | −24.686 | 94.598 | 1.00 | 31.65 |
| 3701 | N | GLN | B | 438 | 48.943 | −24.152 | 93.923 | 1.00 | 29.86 |
| 3702 | CA | GLN | B | 438 | 48.350 | −25.419 | 94.332 | 1.00 | 31.57 |
| 3703 | CB | GLN | B | 438 | 46.843 | −25.222 | 94.617 | 1.00 | 32.61 |
| 3704 | CG | GLN | B | 438 | 46.058 | −26.530 | 94.780 | 1.00 | 28.99 |
| 3705 | CD | GLN | B | 438 | 46.512 | −27.280 | 95.992 | 1.00 | 18.79 |
| 3706 | OE1 | GLN | B | 438 | 45.973 | −27.124 | 97.083 | 1.00 | 7.08 |
| 3707 | NE2 | GLN | B | 438 | 47.529 | −28.071 | 95.818 | 1.00 | 14.08 |
| 3708 | C | GLN | B | 438 | 48.552 | −26.473 | 93.204 | 1.00 | 30.40 |
| 3709 | O | GLN | B | 438 | 48.848 | −27.652 | 93.454 | 1.00 | 20.62 |
| 3710 | N | VAL | B | 439 | 48.372 | −26.026 | 91.963 | 1.00 | 28.89 |
| 3711 | CA | VAL | B | 439 | 48.517 | −26.898 | 90.817 | 1.00 | 32.98 |
| 3712 | CB | VAL | B | 439 | 48.106 | −26.171 | 89.555 | 1.00 | 28.41 |
| 3713 | CG1 | VAL | B | 439 | 48.478 | −26.980 | 88.331 | 1.00 | 29.66 |
| 3714 | CG2 | VAL | B | 439 | 46.618 | −25.936 | 89.597 | 1.00 | 31.84 |
| 3715 | C | VAL | B | 439 | 49.964 | −27.352 | 90.721 | 1.00 | 35.16 |
| 3716 | O | VAL | B | 439 | 50.253 | −28.511 | 90.461 | 1.00 | 37.48 |
| 3717 | N | PHE | B | 440 | 50.877 | −26.425 | 90.930 | 1.00 | 35.36 |
| 3718 | CA | PHE | B | 440 | 52.284 | −26.758 | 90.916 | 1.00 | 37.14 |
| 3719 | CB | PHE | B | 440 | 53.069 | −25.504 | 91.166 | 1.00 | 42.75 |
| 3720 | CG | PHE | B | 440 | 54.504 | −25.742 | 91.492 | 1.00 | 52.63 |
| 3721 | CD1 | PHE | B | 440 | 55.000 | −25.476 | 92.756 | 1.00 | 55.53 |
| 3722 | CD2 | PHE | B | 440 | 55.385 | −26.121 | 90.505 | 1.00 | 58.98 |
| 3723 | CE1 | PHE | B | 440 | 56.369 | −25.579 | 93.012 | 1.00 | 58.23 |
| 3724 | CE2 | PHE | B | 440 | 56.751 | −26.226 | 90.756 | 1.00 | 60.45 |
| 3725 | CZ | PHE | B | 440 | 57.240 | −25.953 | 91.999 | 1.00 | 58.33 |
| 3726 | C | PHE | B | 440 | 52.533 | −27.724 | 92.058 | 1.00 | 38.13 |
| 3727 | O | PHE | B | 440 | 53.277 | −28.683 | 91.916 | 1.00 | 36.27 |
| 3728 | N | ALA | B | 441 | 51.897 | −27.433 | 93.193 | 1.00 | 41.03 |
| 3729 | CA | ALA | B | 441 | 52.006 | −28.231 | 94.404 | 1.00 | 38.74 |
| 3730 | CB | ALA | B | 441 | 51.241 | −27.558 | 95.544 | 1.00 | 38.21 |
| 3731 | C | ALA | B | 441 | 51.492 | −29.653 | 94.202 | 1.00 | 41.20 |
| 3732 | O | ALA | B | 441 | 52.049 | −30.598 | 94.778 | 1.00 | 44.01 |
| 3733 | N | LEU | B | 442 | 50.428 | −29.827 | 93.418 | 1.00 | 39.40 |
| 3734 | CA | LEU | B | 442 | 49.938 | −31.179 | 93.223 | 1.00 | 45.00 |
| 3735 | CB | LEU | B | 442 | 48.402 | −31.291 | 93.408 | 1.00 | 44.36 |
| 3736 | CG | LEU | B | 442 | 47.553 | −30.244 | 92.704 | 1.00 | 48.78 |
| 3737 | CD1 | LEU | B | 442 | 47.759 | −30.335 | 91.219 | 1.00 | 47.07 |
| 3738 | CD2 | LEU | B | 442 | 46.126 | −30.447 | 93.039 | 1.00 | 40.98 |
| 3739 | C | LEU | B | 442 | 50.380 | −31.721 | 91.881 | 1.00 | 49.14 |
| 3740 | O | LEU | B | 442 | 49.609 | −32.344 | 91.144 | 1.00 | 50.63 |
| 3741 | N | ARG | B | 443 | 51.636 | −31.445 | 91.556 | 1.00 | 51.74 |
| 3742 | CA | ARG | B | 443 | 52.214 | −31.973 | 90.344 | 1.00 | 56.94 |
| 3743 | CB | ARG | B | 443 | 52.483 | −30.893 | 89.301 | 1.00 | 64.35 |
| 3744 | CG | ARG | B | 443 | 53.907 | −30.512 | 89.207 | 1.00 | 73.15 |
| 3745 | CD | ARG | B | 443 | 54.252 | −29.690 | 87.962 | 1.00 | 75.45 |
| 3746 | NE | ARG | B | 443 | 55.411 | −28.853 | 88.278 | 1.00 | 84.04 |
| 3747 | CZ | ARG | B | 443 | 56.364 | −29.204 | 89.148 | 1.00 | 84.49 |
| 3748 | NH1 | ARG | B | 443 | 56.292 | −30.364 | 89.778 | 1.00 | 84.77 |
| 3749 | NH2 | ARG | B | 443 | 57.396 | −28.402 | 89.394 | 1.00 | 84.34 |
| 3750 | C | ARG | B | 443 | 53.524 | −32.656 | 90.765 | 1.00 | 58.29 |
| 3751 | O | ARG | B | 443 | 54.010 | −33.553 | 90.087 | 1.00 | 61.51 |
| 3752 | N | LEU | B | 444 | 54.108 | −32.219 | 91.875 | 1.00 | 57.37 |
| 3753 | CA | LEU | B | 444 | 55.303 | −32.866 | 92.387 | 1.00 | 56.22 |
| 3754 | CB | LEU | B | 444 | 56.356 | −31.839 | 92.780 | 1.00 | 46.58 |
| 3755 | CG | LEU | B | 444 | 55.832 | −30.667 | 93.566 | 1.00 | 45.81 |
| 3756 | CD1 | LEU | B | 444 | 55.264 | −31.114 | 94.891 | 1.00 | 40.63 |
| 3757 | CD2 | LEU | B | 444 | 56.956 | −29.725 | 93.812 | 1.00 | 42.09 |
| 3758 | C | LEU | B | 444 | 54.821 | −33.697 | 93.570 | 1.00 | 60.75 |
| 3759 | O | LEU | B | 444 | 55.598 | −34.344 | 94.288 | 1.00 | 64.57 |
| 3760 | N | GLN | B | 445 | 53.512 | −33.628 | 93.795 | 1.00 | 63.20 |
| 3761 | CA | GLN | B | 445 | 52.894 | −34.448 | 94.817 | 1.00 | 65.62 |
| 3762 | CB | GLN | B | 445 | 51.586 | −33.855 | 95.304 | 1.00 | 66.95 |
| 3763 | CG | GLN | B | 445 | 51.441 | −33.848 | 96.784 | 1.00 | 67.17 |
| 3764 | CD | GLN | B | 445 | 50.011 | −33.686 | 97.187 | 1.00 | 78.52 |
| 3765 | OE1 | GLN | B | 445 | 49.706 | −33.566 | 98.373 | 1.00 | 83.85 |
| 3766 | NE2 | GLN | B | 445 | 49.107 | −33.697 | 96.202 | 1.00 | 75.18 |
| 3767 | C | GLN | B | 445 | 52.601 | −35.594 | 93.843 | 1.00 | 68.02 |
| 3768 | O | GLN | B | 445 | 53.156 | −35.627 | 92.722 | 1.00 | 70.38 |
| 3769 | N | ASP | B | 446 | 51.712 | −36.510 | 94.195 | 1.00 | 65.55 |
| 3770 | CA | ASP | B | 446 | 51.463 | −37.605 | 93.275 | 1.00 | 65.65 |
| 3771 | CB | ASP | B | 446 | 51.003 | −38.828 | 94.074 | 1.00 | 68.46 |
| 3772 | CG | ASP | B | 446 | 49.491 | −38.970 | 94.087 | 1.00 | 80.66 |
| 3773 | OD1 | ASP | B | 446 | 48.821 | −38.082 | 94.675 | 1.00 | 85.07 |
| 3774 | OD2 | ASP | B | 446 | 48.973 | −39.950 | 93.481 | 1.00 | 84.14 |
| 3775 | C | ASP | B | 446 | 50.517 | −37.389 | 92.073 | 1.00 | 65.38 |
| 3776 | O | ASP | B | 446 | 50.828 | −37.814 | 90.943 | 1.00 | 65.01 |
| 3777 | N | LYS | B | 447 | 49.413 | −36.674 | 92.268 | 1.00 | 62.03 |
| 3778 | CA | LYS | B | 447 | 48.428 | −36.590 | 91.201 | 1.00 | 57.38 |
| 3779 | CB | LYS | B | 447 | 47.075 | −36.604 | 91.863 | 1.00 | 53.29 |
| 3780 | CG | LYS | B | 447 | 46.954 | −35.520 | 92.915 | 1.00 | 44.15 |
| 3781 | CD | LYS | B | 447 | 46.999 | −36.068 | 94.334 | 1.00 | 53.51 |
| 3782 | CE | LYS | B | 447 | 45.817 | −36.985 | 94.621 | 1.00 | 51.24 |
| 3783 | NZ | LYS | B | 447 | 46.116 | −37.877 | 95.783 | 1.00 | 46.86 |
| 3784 | C | LYS | B | 447 | 48.414 | −35.564 | 90.054 | 1.00 | 56.84 |
| 3785 | O | LYS | B | 447 | 48.875 | −34.437 | 90.218 | 1.00 | 56.28 |
| 3786 | N | LYS | B | 448 | 47.855 | −35.977 | 88.904 | 1.00 | 52.56 |
| 3787 | CA | LYS | B | 448 | 47.745 | −35.114 | 87.730 | 1.00 | 47.66 |
| 3788 | CB | LYS | B | 448 | 48.241 | −35.852 | 86.476 | 1.00 | 52.17 |
| 3789 | CG | LYS | B | 448 | 49.387 | −36.809 | 86.784 | 1.00 | 59.51 |
| 3790 | CD | LYS | B | 448 | 50.336 | −37.016 | 85.619 | 1.00 | 72.25 |
| 3791 | CE | LYS | B | 448 | 51.569 | −37.802 | 86.080 | 1.00 | 76.88 |
| 3792 | NZ | LYS | B | 448 | 52.651 | −37.852 | 85.038 | 1.00 | 83.91 |
| 3793 | C | LYS | B | 448 | 46.272 | −34.712 | 87.573 | 1.00 | 41.95 |
| 3794 | O | LYS | B | 448 | 45.432 | −35.114 | 88.367 | 1.00 | 36.65 |
| 3795 | N | LEU | B | 449 | 45.964 | −33.933 | 86.542 | 1.00 | 36.83 |
| 3796 | CA | LEU | B | 449 | 44.600 | −33.475 | 86.317 | 1.00 | 29.86 |
| 3797 | CB | LEU | B | 449 | 44.586 | −31.939 | 86.315 | 1.00 | 29.17 |
| 3798 | CG | LEU | B | 449 | 45.319 | −31.191 | 87.441 | 1.00 | 21.55 |
| 3799 | CD1 | LEU | B | 449 | 45.698 | −29.739 | 87.026 | 1.00 | 13.48 |
| 3800 | CD2 | LEU | B | 449 | 44.414 | −31.191 | 88.666 | 1.00 | 25.46 |
| 3801 | C | LEU | B | 449 | 44.013 | −33.966 | 85.001 | 1.00 | 25.36 |
| 3802 | O | LEU | B | 449 | 44.727 | −34.159 | 84.017 | 1.00 | 20.95 |
| 3803 | N | PRO | B | 450 | 42.693 | −34.159 | 84.959 | 1.00 | 23.78 |
| 3804 | CD | PRO | B | 450 | 41.633 | −34.036 | 85.973 | 1.00 | 15.08 |
| 3805 | CA | PRO | B | 450 | 42.158 | −34.616 | 83.670 | 1.00 | 24.94 |
| 3806 | GB | PRO | B | 450 | 40.642 | −34.726 | 83.918 | 1.00 | 19.41 |
| 3807 | CG | PRO | B | 450 | 40.405 | −33.860 | 85.110 | 1.00 | 19.53 |
| 3808 | C | PRO | B | 450 | 42.498 | −33.588 | 82.618 | 1.00 | 27.11 |
| 3809 | O | PRO | B | 450 | 42.523 | −32.384 | 82.933 | 1.00 | 26.09 |
| 3810 | N | PRO | B | 451 | 42.775 | −34.040 | 81.366 | 1.00 | 29.87 |
| 3811 | CD | PRO | B | 451 | 42.281 | −35.332 | 80.852 | 1.00 | 26.06 |
| 3812 | CA | PRO | B | 451 | 43.127 | −33.160 | 80.233 | 1.00 | 27.77 |
| 3813 | CB | PRO | B | 451 | 42.705 | −33.991 | 79.011 | 1.00 | 25.33 |
| 3814 | CG | PRO | B | 451 | 41.672 | −34.911 | 79.548 | 1.00 | 28.40 |
| 3815 | C | PRO | B | 451 | 42.537 | −31.742 | 80.216 | 1.00 | 23.36 |
| 3816 | O | PRO | B | 451 | 43.259 | −30.771 | 80.128 | 1.00 | 27.32 |
| 3817 | N | LEU | B | 452 | 41.227 | −31.635 | 80.298 | 1.00 | 19.34 |
| 3818 | CA | LEU | B | 452 | 40.610 | −30.336 | 80.289 | 1.00 | 18.25 |
| 3819 | CB | LEU | B | 452 | 39.095 | −30.464 | 80.525 | 1.00 | 14.12 |
| 3820 | CG | LEU | B | 452 | 38.325 | −29.138 | 80.411 | 1.00 | 13.10 |
| 3821 | CD1 | LEU | B | 452 | 38.711 | −28.489 | 79.089 | 1.00 | 18.06 |
| 3822 | CD2 | LEU | B | 452 | 36.855 | −29.329 | 80.488 | 1.00 | 5.25 |
| 3823 | C | LEU | B | 452 | 41.244 | −29.424 | 81.346 | 1.00 | 19.73 |
| 3824 | O | LEU | B | 452 | 41.904 | −28.434 | 80.990 | 1.00 | 18.11 |
| 3825 | N | LEU | B | 453 | 41.033 | −29.737 | 82.629 | 1.00 | 18.83 |
| 3826 | CA | LEU | B | 453 | 41.602 | −28.927 | 83.707 | 1.00 | 19.53 |
| 3827 | GB | LEU | B | 453 | 41.444 | −29.610 | 85.060 | 1.00 | 13.81 |
| 3828 | CG | LEU | B | 453 | 40.005 | −29.927 | 85.467 | 1.00 | 22.28 |
| 3829 | CD1 | LEU | B | 453 | 40.019 | −30.669 | 86.841 | 1.00 | 12.32 |
| 3830 | CD2 | LEU | B | 453 | 39.206 | −28.627 | 85.529 | 1.00 | 10.46 |
| 3831 | C | LEU | B | 453 | 43.081 | −28.680 | 83.463 | 1.00 | 22.21 |
| 3832 | O | LEU | B | 453 | 43.583 | −27.565 | 83.690 | 1.00 | 23.09 |
| 3833 | N | SER | B | 454 | 43.770 | −29.728 | 83.011 | 1.00 | 22.76 |
| 3834 | CA | SER | B | 454 | 45.193 | −29.644 | 82.712 | 1.00 | 23.20 |
| 3835 | CB | SER | B | 454 | 45.702 | −31.022 | 82.293 | 1.00 | 28.99 |
| 3836 | OG | SER | B | 454 | 47.115 | −30.997 | 82.118 | 1.00 | 31.87 |
| 3837 | C | SER | B | 454 | 45.494 | −28.593 | 81.591 | 1.00 | 21.00 |
| 3838 | O | SER | B | 454 | 46.543 | −27.934 | 81.598 | 1.00 | 16.82 |
| 3839 | N | GLU | B | 455 | 44.582 | −28.469 | 80.635 | 1.00 | 17.12 |
| 3840 | CA | GLU | B | 455 | 44.726 | −27.496 | 79.576 | 1.00 | 24.84 |
| 3841 | CB | GLU | B | 455 | 43.526 | −27.501 | 78.662 | 1.00 | 26.84 |
| 3842 | CG | GLU | B | 455 | 43.700 | −28.184 | 77.353 | 1.00 | 18.84 |
| 3843 | CD | GLU | B | 455 | 42.476 | −27.976 | 76.444 | 1.00 | 38.40 |
| 3844 | OE1 | GLU | B | 455 | 42.120 | −28.905 | 75.655 | 1.00 | 30.86 |
| 3845 | OE2 | GLU | B | 455 | 41.859 | −26.868 | 76.516 | 1.00 | 26.15 |
| 3846 | C | GLU | B | 455 | 44.794 | −26.119 | 80.183 | 1.00 | 30.03 |
| 3847 | O | GLU | B | 455 | 45.732 | −25.375 | 79.938 | 1.00 | 33.86 |
| 3848 | N | ILE | B | 456 | 43.788 | −25.790 | 80.983 | 1.00 | 32.39 |
| 3849 | CA | ILE | B | 456 | 43.691 | −24.492 | 81.643 | 1.00 | 33.77 |
| 3850 | CB | ILE | B | 456 | 42.298 | −24.352 | 82.263 | 1.00 | 37.16 |
| 3851 | CG2 | ILE | B | 456 | 42.093 | −22.971 | 82.780 | 1.00 | 44.25 |
| 3852 | CG1 | ILE | B | 456 | 41.238 | −24.668 | 81.231 | 1.00 | 32.75 |
| 3853 | CD1 | ILE | B | 456 | 39.825 | −24.585 | 81.833 | 1.00 | 44.84 |
| 3854 | C | ILE | B | 456 | 44.703 | −24.174 | 82.755 | 1.00 | 31.98 |
| 3855 | O | ILE | B | 456 | 45.170 | −23.053 | 82.861 | 1.00 | 22.75 |
| 3856 | N | TRP | B | 457 | 45.057 | −25.178 | 83.552 | 1.00 | 35.21 |
| 3857 | CA | TRP | B | 457 | 45.928 | −24.959 | 84.705 | 1.00 | 40.82 |
| 3858 | CB | TRP | B | 457 | 45.155 | −25.417 | 85.942 | 1.00 | 38.48 |
| 3859 | CG | TRP | B | 457 | 43.835 | −24.729 | 86.039 | 1.00 | 22.11 |
| 3860 | CD2 | TRP | B | 457 | 42.656 | −25.242 | 86.652 | 1.00 | 19.60 |
| 3861 | CE2 | TRP | B | 457 | 41.699 | −24.212 | 86.632 | 1.00 | 19.91 |
| 3862 | CE3 | TRP | B | 457 | 42.315 | −26.478 | 87.214 | 1.00 | 16.21 |
| 3863 | CD1 | TRP | B | 457 | 43.567 | −23.463 | 85.673 | 1.00 | 5.25 |
| 3864 | NE1 | TRP | B | 457 | 42.301 | −23.137 | 86.023 | 1.00 | 9.21 |
| 3865 | CZ2 | TRP | B | 457 | 40.404 | −24.358 | 87.167 | 1.00 | 21.85 |
| 3866 | CZ3 | TRP | B | 457 | 41.031 | −26.639 | 87.748 | 1.00 | 20.82 |
| 3867 | CH2 | TRP | B | 457 | 40.087 | −25.578 | 87.717 | 1.00 | 28.52 |
| 3868 | C | TRP | B | 457 | 47.370 | −25.460 | 84.769 | 1.00 | 43.65 |
| 3869 | O | TRP | B | 457 | 48.235 | −24.810 | 85.323 | 1.00 | 42.42 |
| 3870 | N | ASP | B | 458 | 47.616 | −26.629 | 84.223 | 1.00 | 49.96 |
| 3871 | CA | ASP | B | 458 | 48.943 | −27.233 | 84.202 | 1.00 | 57.57 |
| 3872 | CB | ASP | B | 458 | 48.814 | −28.592 | 83.517 | 1.00 | 53.25 |
| 3873 | CG | ASP | B | 458 | 48.626 | −29.711 | 84.456 | 1.00 | 53.35 |
| 3874 | OD1 | ASP | B | 458 | 48.604 | −29.447 | 85.668 | 1.00 | 53.90 |
| 3875 | OD2 | ASP | B | 458 | 48.517 | −30.865 | 83.990 | 1.00 | 47.19 |
| 3876 | C | ASP | B | 458 | 49.880 | −26.432 | 83.291 | 1.00 | 66.03 |
| 3877 | O | ASP | B | 458 | 51.116 | −26.670 | 83.226 | 1.00 | 69.33 |
| 3878 | N | VAL | B | 459 | 49.313 | −25.415 | 82.668 | 1.00 | 72.79 |
| 3879 | CA | VAL | B | 459 | 49.997 | −24.823 | 81.543 | 1.00 | 77.10 |
| 3880 | CB | VAL | B | 459 | 49.706 | −25.870 | 80.387 | 1.00 | 82.78 |
| 3881 | CG1 | VAL | B | 459 | 49.387 | −25.195 | 79.068 | 1.00 | 79.26 |
| 3882 | CG2 | VAL | B | 459 | 50.805 | −26.921 | 80.294 | 1.00 | 77.57 |
| 3883 | C | VAL | B | 459 | 49.287 | −23.512 | 81.206 | 1.00 | 78.30 |
| 3884 | O | VAL | B | 459 | 48.034 | −23.590 | 81.120 | 1.00 | 74.12 |
| 3885 | OT | VAL | B | 459 | 49.966 | −22.483 | 80.966 | 1.00 | 85.25 |
| 3886 | | VAL | B | 459 | | | | | |
| 3887 | CB | ALA | C | −3 | 54.311 | −13.946 | 114.812 | 1.00 | 76.41 |
| 3888 | C | ALA | C | −3 | 56.087 | −12.625 | 113.749 | 1.00 | 73.80 |
| 3889 | O | ALA | C | −3 | 57.155 | −12.786 | 113.165 | 1.00 | 76.36 |
| 3890 | N | ALA | C | −3 | 55.919 | −12.651 | 116.146 | 1.00 | 69.92 |
| 3891 | CA | ALA | C | −3 | 55.735 | −13.496 | 114.929 | 1.00 | 71.93 |
| 3892 | N | LEU | C | −2 | 55.158 | −11.736 | 113.386 | 1.00 | 72.81 |
| 3893 | CA | LEU | C | −2 | 55.342 | −10.770 | 112.302 | 1.00 | 69.21 |
| 3894 | CB | LEU | C | −2 | 53.991 | −10.325 | 111.752 | 1.00 | 63.26 |
| 3895 | CG | LEU | C | −2 | 53.533 | −11.140 | 110.563 | 1.00 | 54.22 |
| 3896 | CD1 | LEU | C | −2 | 52.080 | −10.849 | 110.306 | 1.00 | 49.29 |
| 3897 | CD2 | LEU | C | −2 | 54.399 | −10.811 | 109.369 | 1.00 | 49.99 |
| 3898 | C | LEU | C | −2 | 55.993 | −9.591 | 113.019 | 1.00 | 69.59 |
| 3899 | O | LEU | C | −2 | 55.292 | −8.692 | 113.507 | 1.00 | 73.65 |
| 3900 | N | ALA | C | −1 | 57.318 | −9.580 | 113.095 | 1.00 | 66.78 |
| 3901 | CA | ALA | C | −1 | 58.036 | −8.509 | 113.802 | 1.00 | 69.32 |
| 3902 | CB | ALA | C | −1 | 57.372 | −8.206 | 115.172 | 1.00 | 60.13 |
| 3903 | C | ALA | C | −1 | 59.414 | −9.108 | 114.000 | 1.00 | 67.12 |
| 3904 | O | ALA | C | −1 | 60.454 | −8.491 | 113.732 | 1.00 | 71.81 |
| 3905 | N | ALA | C | 0 | 59.389 | −10.329 | 114.513 | 1.00 | 63.04 |
| 3906 | CA | ALA | C | 0 | 60.592 | −11.111 | 114.692 | 1.00 | 56.99 |
| 3907 | CB | ALA | C | 0 | 60.285 | −12.293 | 115.582 | 1.00 | 51.89 |
| 3908 | C | ALA | C | 0 | 60.969 | −11.582 | 113.261 | 1.00 | 53.09 |
| 3909 | O | ALA | C | 0 | 62.120 | −11.486 | 112.833 | 1.00 | 48.76 |
| 3910 | N | ARG | C | 1 | 59.982 | −12.037 | 112.493 | 1.00 | 51.49 |
| 3911 | CA | ARG | C | 1 | 60.244 | −12.530 | 111.150 | 1.00 | 46.31 |
| 3912 | CB | ARG | C | 1 | 58.969 | −13.173 | 110.567 | 1.00 | 53.19 |
| 3913 | CG | ARG | C | 1 | 58.887 | −14.686 | 110.805 | 1.00 | 46.35 |
| 3914 | CD | ARG | C | 1 | 57.488 | −15.191 | 111.211 | 1.00 | 56.12 |
| 3915 | NE | ARG | C | 1 | 56.938 | −16.060 | 110.172 | 1.00 | 62.40 |
| 3916 | CZ | ARG | C | 1 | 56.402 | −15.603 | 109.038 | 1.00 | 70.46 |
| 3917 | NH1 | ARG | C | 1 | 56.342 | −14.295 | 108.832 | 1.00 | 70.27 |
| 3918 | NH2 | ARG | C | 1 | 55.965 | −16.439 | 108.090 | 1.00 | 60.91 |
| 3919 | C | ARG | C | 1 | 60.752 | −11.403 | 110.254 | 1.00 | 45.64 |
| 3920 | O | ARG | C | 1 | 61.385 | −11.646 | 109.208 | 1.00 | 44.18 |
| 3921 | N | HIS | C | 2 | 60.520 | −10.167 | 110.698 | 1.00 | 40.66 |
| 3922 | CA | HIS | C | 2 | 60.909 | −8.991 | 109.926 | 1.00 | 35.21 |
| 3923 | CB | HIS | C | 2 | 59.663 | −8.408 | 109.271 | 1.00 | 35.35 |
| 3924 | CG | HIS | C | 2 | 58.977 | −9.362 | 108.341 | 1.00 | 36.61 |
| 3925 | CD2 | HIS | C | 2 | 58.701 | −9.277 | 107.017 | 1.00 | 39.53 |
| 3926 | ND1 | HIS | C | 2 | 58.499 | −10.589 | 108.749 | 1.00 | 39.75 |
| 3927 | CE1 | HIS | C | 2 | 57.963 | −11.218 | 107.719 | 1.00 | 47.92 |
| 3928 | NE2 | HIS | C | 2 | 58.073 | −10.441 | 106.655 | 1.00 | 45.80 |
| 3929 | C | HIS | C | 2 | 61.612 | −7.918 | 110.743 | 1.00 | 30.30 |
| 3930 | O | HIS | C | 2 | 61.117 | −6.806 | 110.857 | 1.00 | 23.01 |
| 3931 | N | LYS | C | 3 | 62.789 | −8.257 | 111.263 | 1.00 | 32.28 |
| 3932 | CA | LYS | C | 3 | 63.564 | −7.371 | 112.122 | 1.00 | 32.74 |
| 3933 | CB | LYS | C | 3 | 64.897 | −8.049 | 112.468 | 1.00 | 39.84 |
| 3934 | CG | LYS | C | 3 | 65.426 | −7.709 | 113.849 | 1.00 | 63.43 |
| 3935 | CD | LYS | C | 3 | 66.560 | −8.657 | 114.292 | 1.00 | 81.08 |
| 3936 | CE | LYS | C | 3 | 67.102 | −8.316 | 115.700 | 1.00 | 83.76 |
| 3937 | NZ | LYS | C | 3 | 68.174 | −9.293 | 116.108 | 1.00 | 81.79 |
| 3938 | C | LYS | C | 3 | 63.793 | −5.948 | 111.593 | 1.00 | 33.33 |
| 3939 | O | LYS | C | 3 | 63.344 | −4.979 | 112.227 | 1.00 | 28.13 |
| 3940 | N | ILE | C | 4 | 64.470 | −5.822 | 110.441 | 1.00 | 31.52 |
| 3941 | CA | ILE | C | 4 | 64.768 | −4.505 | 109.868 | 1.00 | 31.05 |
| 3942 | CB | ILE | C | 4 | 65.504 | −4.602 | 108.524 | 1.00 | 27.45 |
| 3943 | CG2 | ILE | C | 4 | 65.802 | −3.201 | 108.015 | 1.00 | 15.44 |
| 3944 | CG1 | ILE | C | 4 | 66.793 | −5.400 | 108.658 | 1.00 | 15.17 |
| 3945 | CD1 | ILE | C | 4 | 67.456 | −5.634 | 107.333 | 1.00 | 5.25 |
| 3946 | C | ILE | C | 4 | 63.487 | −3.703 | 109.601 | 1.00 | 35.03 |
| 3947 | O | ILE | C | 4 | 63.380 | −2.504 | 109.892 | 1.00 | 38.81 |
| 3948 | N | LEU | C | 5 | 62.501 | −4.367 | 109.036 | 1.00 | 32.79 |
| 3949 | CA | LEU | C | 5 | 61.284 | −3.666 | 108.760 | 1.00 | 31.98 |
| 3950 | CB | LEU | C | 5 | 60.278 | −4.606 | 108.116 | 1.00 | 28.91 |
| 3951 | CG | LEU | C | 5 | 59.032 | −3.918 | 107.587 | 1.00 | 21.35 |
| 3952 | CD1 | LEU | C | 5 | 59.475 | −3.018 | 106.479 | 1.00 | 27.75 |
| 3953 | CD2 | LEU | C | 5 | 58.025 | −4.921 | 107.073 | 1.00 | 28.10 |
| 3954 | C | LEU | C | 5 | 60.732 | −3.127 | 110.055 | 1.00 | 34.05 |
| 3955 | O | LEU | C | 5 | 60.301 | −1.981 | 110.131 | 1.00 | 37.58 |
| 3956 | N | HIS | C | 6 | 60.743 | −3.959 | 111.089 | 1.00 | 37.37 |
| 3957 | CA | HIS | C | 6 | 60.210 | −3.543 | 112.385 | 1.00 | 40.64 |
| 3958 | CB | HIS | C | 6 | 60.402 | −4.640 | 113.451 | 1.00 | 45.69 |
| 3959 | CG | HIS | C | 6 | 59.721 | −4.343 | 114.764 | 1.00 | 52.81 |
| 3960 | CD2 | HIS | C | 6 | 60.134 | −3.641 | 115.848 | 1.00 | 48.43 |
| 3961 | ND1 | HIS | C | 6 | 58.425 | −4.734 | 115.044 | 1.00 | 56.64 |
| 3962 | CE1 | HIS | C | 6 | 58.071 | −4.283 | 116.233 | 1.00 | 47.70 |
| 3963 | NE2 | HIS | C | 6 | 59.091 | −3.614 | 116.743 | 1.00 | 45.83 |
| 3964 | C | HIS | C | 6 | 60.910 | −2.284 | 112.853 | 1.00 | 42.95 |
| 3965 | O | HIS | C | 6 | 60.257 | −1.390 | 113.365 | 1.00 | 46.57 |
| 3966 | N | ARG | C | 7 | 62.233 | −2.237 | 112.667 | 1.00 | 41.77 |
| 3967 | CA | ARG | C | 7 | 63.072 | −1.112 | 113.064 | 1.00 | 39.07 |
| 3968 | CB | ARG | C | 7 | 64.536 | −1.425 | 112.743 | 1.00 | 47.69 |
| 3969 | CG | ARG | C | 7 | 65.542 | −0.516 | 113.444 | 1.00 | 53.50 |
| 3970 | CD | ARG | C | 7 | 66.864 | −0.572 | 112.734 | 1.00 | 59.29 |
| 3971 | NE | ARG | C | 7 | 66.855 | 0.241 | 111.514 | 1.00 | 74.08 |
| 3972 | CZ | ARG | C | 7 | 67.445 | −0.109 | 110.371 | 1.00 | 78.20 |
| 3973 | NH1 | ARG | C | 7 | 68.084 | −1.271 | 110.290 | 1.00 | 83.39 |
| 3974 | NH2 | ARG | C | 7 | 67.427 | 0.721 | 109.320 | 1.00 | 72.71 |
| 3975 | C | ARG | C | 7 | 62.663 | 0.199 | 112.376 | 1.00 | 36.66 |
| 3976 | O | ARG | C | 7 | 62.613 | 1.274 | 113.005 | 1.00 | 34.51 |
| 3977 | N | LEU | C | 8 | 62.376 | 0.124 | 111.082 | 1.00 | 32.76 |
| 3978 | CA | LEU | C | 8 | 61.949 | 1.330 | 110.368 | 1.00 | 33.44 |
| 3979 | CB | LEU | C | 8 | 61.714 | 1.050 | 108.882 | 1.00 | 26.26 |
| 3980 | CG | LEU | C | 8 | 62.997 | 0.774 | 108.087 | 1.00 | 26.09 |
| 3981 | CD1 | LEU | C | 8 | 62.728 | 0.308 | 106.677 | 1.00 | 18.55 |
| 3982 | CD2 | LEU | C | 8 | 63.796 | 2.047 | 108.092 | 1.00 | 30.16 |
| 3983 | C | LEU | C | 8 | 60.678 | 1.904 | 110.965 | 1.00 | 33.67 |
| 3984 | O | LEU | C | 8 | 60.523 | 3.110 | 111.030 | 1.00 | 35.99 |
| 3985 | N | LEU | C | 9 | 59.762 | 1.056 | 111.410 | 1.00 | 31.98 |
| 3986 | CA | LEU | C | 9 | 58.538 | 1.589 | 111.984 | 1.00 | 35.05 |
| 3987 | CB | LEU | C | 9 | 57.514 | 0.489 | 112.090 | 1.00 | 25.12 |
| 3988 | CG | LEU | C | 9 | 57.147 | 0.051 | 110.716 | 1.00 | 20.67 |
| 3989 | CD1 | LEU | C | 9 | 56.876 | −1.425 | 110.711 | 1.00 | 26.48 |
| 3990 | CD2 | LEU | C | 9 | 55.959 | 0.884 | 110.274 | 1.00 | 20.75 |
| 3991 | C | LEU | C | 9 | 58.717 | 2.244 | 113.349 | 1.00 | 40.45 |
| 3992 | O | LEU | C | 9 | 57.824 | 2.944 | 113.825 | 1.00 | 39.35 |
| 3993 | N | GLN | C | 10 | 59.870 | 2.025 | 113.974 | 1.00 | 46.16 |
| 3994 | CA | GLN | C | 10 | 60.125 | 2.585 | 115.300 | 1.00 | 52.79 |
| 3995 | CB | GLN | C | 10 | 60.679 | 1.510 | 116.251 | 1.00 | 49.83 |
| 3996 | CG | GLN | C | 10 | 59.666 | 0.491 | 116.727 | 1.00 | 45.94 |
| 3997 | CD | GLN | C | 10 | 60.162 | −0.289 | 117.943 | 1.00 | 46.74 |
| 3998 | OE1 | GLN | C | 10 | 61.137 | −1.038 | 117.865 | 1.00 | 47.34 |
| 3999 | NE2 | GLN | C | 10 | 59.494 | −0.099 | 119.078 | 1.00 | 45.75 |
| 4000 | C | GLN | C | 10 | 61.081 | 3.770 | 115.293 | 1.00 | 57.32 |
| 4001 | O | GLN | C | 10 | 61.180 | 4.476 | 116.298 | 1.00 | 58.42 |
| 4002 | N | GLU | C | 11 | 61.782 | 3.992 | 114.179 | 1.00 | 60.53 |
| 4003 | CA | GLU | C | 11 | 62.709 | 5.114 | 114.127 | 1.00 | 66.82 |
| 4004 | CB | GLU | C | 11 | 63.652 | 5.000 | 112.931 | 1.00 | 64.61 |
| 4005 | CG | GLU | C | 11 | 63.118 | 5.565 | 111.627 | 1.00 | 75.77 |
| 4006 | CD | GLU | C | 11 | 64.147 | 5.526 | 110.498 | 1.00 | 80.79 |
| 4007 | OE1 | GLU | C | 11 | 64.583 | 4.419 | 110.101 | 1.00 | 79.00 |
| 4008 | OE2 | GLU | C | 11 | 64.529 | 6.611 | 110.004 | 1.00 | 85.25 |
| 4009 | C | GLU | C | 11 | 61.934 | 6.431 | 114.051 | 1.00 | 71.59 |
| 4010 | O | GLU | C | 11 | 62.520 | 7.506 | 114.216 | 1.00 | 74.45 |
| 4011 | N | GLY | C | 12 | 60.619 | 6.340 | 113.816 | 1.00 | 73.58 |
| 4012 | CA | GLY | C | 12 | 59.781 | 7.531 | 113.735 | 1.00 | 73.73 |
| 4013 | C | GLY | C | 12 | 59.669 | 8.271 | 115.065 | 1.00 | 73.31 |
| 4014 | O | GLY | C | 12 | 60.274 | 7.811 | 116.063 | 1.00 | 70.87 |
| 4015 | OT | GLY | C | 12 | 58.974 | 9.314 | 115.117 | 1.00 | 73.17 |
| 4016 | | GLY | C | 12 | | | | | |
| 4017 | CB | ARG | D | 1 | 46.885 | −26.379 | 71.137 | 1.00 | 46.99 |
| 4018 | CG | ARG | D | 1 | 46.803 | −25.652 | 69.764 | 1.00 | 48.39 |
| 4019 | CD | ARG | D | 1 | 46.249 | −24.242 | 69.898 | 1.00 | 55.16 |
| 4020 | NE | ARG | D | 1 | 44.834 | −24.129 | 69.566 | 1.00 | 57.32 |
| 4021 | CZ | ARG | D | 1 | 43.994 | −23.298 | 70.171 | 1.00 | 59.78 |
| 4022 | NH1 | ARG | D | 1 | 44.417 | −22.515 | 71.155 | 1.00 | 58.58 |
| 4023 | NH2 | ARG | D | 1 | 42.740 | −23.226 | 69.759 | 1.00 | 56.99 |
| 4024 | C | ARG | D | 1 | 46.009 | −25.804 | 73.394 | 1.00 | 52.97 |
| 4025 | O | ARG | D | 1 | 46.351 | −26.313 | 74.496 | 1.00 | 55.64 |
| 4026 | N | ARG | D | 1 | 48.363 | −25.358 | 72.954 | 1.00 | 43.08 |
| 4027 | CA | ARG | D | 1 | 47.013 | −25.417 | 72.355 | 1.00 | 50.28 |
| 4028 | N | HIS | D | 2 | 44.763 | −25.584 | 73.045 | 1.00 | 53.02 |
| 4029 | CA | HIS | D | 2 | 43.746 | −25.866 | 73.986 | 1.00 | 47.41 |
| 4030 | CB | HIS | D | 2 | 43.273 | −24.534 | 74.534 | 1.00 | 45.72 |
| 4031 | CG | HIS | D | 2 | 44.278 | −23.864 | 75.434 | 1.00 | 49.31 |
| 4032 | CD2 | HIS | D | 2 | 44.255 | −23.654 | 76.776 | 1.00 | 47.68 |
| 4033 | ND1 | HIS | D | 2 | 45.484 | −23.349 | 74.995 | 1.00 | 54.30 |
| 4034 | CE1 | HIS | D | 2 | 46.156 | −22.857 | 76.026 | 1.00 | 49.22 |
| 4035 | NE2 | HIS | D | 2 | 45.430 | −23.032 | 77.120 | 1.00 | 38.45 |
| 4036 | C | HIS | D | 2 | 42.710 | −26.589 | 73.193 | 1.00 | 48.14 |
| 4037 | O | HIS | D | 2 | 41.662 | −26.036 | 72.884 | 1.00 | 47.23 |
| 4038 | N | LYS | D | 3 | 43.036 | −27.825 | 72.812 | 1.00 | 46.92 |
| 4039 | CA | LYS | D | 3 | 42.103 | −28.609 | 72.023 | 1.00 | 43.56 |
| 4040 | CB | LYS | D | 3 | 42.672 | −30.006 | 71.775 | 1.00 | 46.80 |
| 4041 | CG | LYS | D | 3 | 43.825 | −30.070 | 70.783 | 1.00 | 49.39 |
| 4042 | CD | LYS | D | 3 | 44.951 | −30.977 | 71.272 | 1.00 | 60.15 |
| 4043 | CE | LYS | D | 3 | 44.485 | −32.407 | 71.569 | 1.00 | 65.20 |
| 4044 | NZ | LYS | D | 3 | 45.623 | −33.253 | 72.085 | 1.00 | 55.18 |
| 4045 | C | LYS | D | 3 | 40.696 | −28.706 | 72.638 | 1.00 | 43.26 |
| 4046 | O | LYS | D | 3 | 39.721 | −28.297 | 71.998 | 1.00 | 43.79 |
| 4047 | N | ILE | D | 4 | 40.578 | −29.185 | 73.879 | 1.00 | 40.38 |
| 4048 | CA | ILE | D | 4 | 39.256 | −29.346 | 74.456 | 1.00 | 39.52 |
| 4049 | CB | ILE | D | 4 | 39.312 | −30.051 | 75.802 | 1.00 | 34.88 |
| 4050 | CG2 | ILE | D | 4 | 37.906 | −30.280 | 76.289 | 1.00 | 26.09 |
| 4051 | CG1 | ILE | D | 4 | 40.057 | −31.381 | 75.664 | 1.00 | 27.24 |
| 4052 | CD1 | ILE | D | 4 | 40.402 | −32.080 | 76.974 | 1.00 | 15.88 |
| 4053 | C | ILE | D | 4 | 38.486 | −28.046 | 74.623 | 1.00 | 42.72 |
| 4054 | O | ILE | D | 4 | 37.365 | −27.874 | 74.104 | 1.00 | 42.17 |
| 4055 | N | LEU | D | 5 | 39.104 | −27.119 | 75.345 | 1.00 | 43.51 |
| 4056 | CA | LEU | D | 5 | 38.487 | −25.818 | 75.604 | 1.00 | 41.72 |
| 4057 | CB | LEU | D | 5 | 39.492 | −24.902 | 76.303 | 1.00 | 34.56 |
| 4058 | CG | LEU | D | 5 | 38.972 | −23.526 | 76.646 | 1.00 | 12.80 |
| 4059 | CD1 | LEU | D | 5 | 37.986 | −23.667 | 77.770 | 1.00 | 23.18 |
| 4060 | CD2 | LEU | D | 5 | 40.111 | −22.651 | 77.080 | 1.00 | 18.96 |
| 4061 | C | LEU | D | 5 | 38.016 | −25.190 | 74.303 | 1.00 | 41.97 |
| 4062 | O | LEU | D | 5 | 36.878 | −24.766 | 74.163 | 1.00 | 42.53 |
| 4063 | N | HIS | D | 6 | 38.914 | −25.134 | 73.337 | 1.00 | 46.29 |
| 4064 | CA | HIS | D | 6 | 38.587 | −24.574 | 72.027 | 1.00 | 46.35 |
| 4065 | CB | HIS | D | 6 | 39.770 | −24.777 | 71.085 | 1.00 | 48.78 |
| 4066 | CG | HIS | D | 6 | 39.536 | −24.257 | 69.702 | 1.00 | 49.82 |
| 4067 | CD2 | HIS | D | 6 | 40.214 | −23.348 | 68.965 | 1.00 | 52.01 |
| 4068 | ND1 | HIS | D | 6 | 38.500 | −24.689 | 68.904 | 1.00 | 52.61 |
| 4069 | CE1 | HIS | D | 6 | 38.548 | −24.073 | 67.739 | 1.00 | 37.70 |
| 4070 | NE2 | HIS | D | 6 | 39.581 | −23.252 | 67.752 | 1.00 | 45.90 |
| 4071 | C | HIS | D | 6 | 37.369 | −25.306 | 71.480 | 1.00 | 43.76 |
| 4072 | O | HIS | D | 6 | 36.400 | −24.687 | 71.069 | 1.00 | 44.39 |
| 4073 | N | ARG | D | 7 | 37.449 | −26.631 | 71.492 | 1.00 | 40.72 |
| 4074 | CA | ARG | D | 7 | 36.385 | −27.490 | 71.019 | 1.00 | 42.50 |
| 4075 | CB | ARG | D | 7 | 36.724 | −28.940 | 71.370 | 1.00 | 52.07 |
| 4076 | CG | ARG | D | 7 | 35.783 | −29.963 | 70.763 | 1.00 | 60.29 |
| 4077 | CD | ARG | D | 7 | 36.198 | −31.348 | 71.144 | 1.00 | 69.09 |
| 4078 | NE | ARG | D | 7 | 35.732 | −32.356 | 70.195 | 1.00 | 82.74 |
| 4079 | CZ | ARG | D | 7 | 36.078 | −33.641 | 70.259 | 1.00 | 85.02 |
| 4080 | NH1 | ARG | D | 7 | 36.883 | −34.044 | 71.232 | 1.00 | 84.76 |
| 4081 | NH2 | ARG | D | 7 | 35.642 | −34.516 | 69.345 | 1.00 | 84.04 |
| 4082 | C | ARG | D | 7 | 35.028 | −27.110 | 71.615 | 1.00 | 41.71 |
| 4083 | O | ARG | D | 7 | 34.006 | −27.079 | 70.917 | 1.00 | 41.93 |
| 4084 | N | LEU | D | 8 | 35.015 | −26.826 | 72.917 | 1.00 | 40.13 |
| 4085 | CA | LEU | D | 8 | 33.780 | −26.424 | 73.610 | 1.00 | 34.27 |
| 4086 | CB | LEU | D | 8 | 34.005 | −26.284 | 75.111 | 1.00 | 22.98 |
| 4087 | CG | LEU | D | 8 | 34.147 | −27.629 | 75.837 | 1.00 | 29.94 |
| 4088 | CD1 | LEU | D | 8 | 34.817 | −27.430 | 77.210 | 1.00 | 8.95 |
| 4089 | CD2 | LEU | D | 8 | 32.754 | −28.265 | 75.961 | 1.00 | 11.55 |
| 4090 | C | LEU | D | 8 | 33.263 | −25.114 | 73.074 | 1.00 | 33.85 |
| 4091 | O | LEU | D | 8 | 32.060 | −24.992 | 72.874 | 1.00 | 35.85 |
| 4092 | N | LEU | D | 9 | 34.163 | −24.153 | 72.830 | 1.00 | 30.89 |
| 4093 | CA | LEU | D | 9 | 33.758 | −22.867 | 72.308 | 1.00 | 31.74 |
| 4094 | CB | LEU | D | 9 | 34.987 | −22.001 | 72.118 | 1.00 | 28.12 |
| 4095 | CG | LEU | D | 9 | 35.548 | −21.424 | 73.414 | 1.00 | 28.94 |
| 4096 | CD1 | LEU | D | 9 | 37.001 | −20.987 | 73.211 | 1.00 | 30.37 |
| 4097 | CD2 | LEU | D | 9 | 34.660 | −20.246 | 73.863 | 1.00 | 24.09 |
| 4098 | C | LEU | D | 9 | 32.938 | −22.970 | 71.013 | 1.00 | 35.76 |
| 4099 | O | LEU | D | 9 | 32.121 | −22.106 | 70.710 | 1.00 | 31.76 |
| 4100 | N | GLN | D | 10 | 33.108 | −24.049 | 70.265 | 1.00 | 43.66 |
| 4101 | CA | GLN | D | 10 | 32.350 | −24.174 | 69.039 | 1.00 | 55.74 |
| 4102 | CB | GLN | D | 10 | 33.028 | −25.151 | 68.108 | 1.00 | 53.38 |
| 4103 | CG | GLN | D | 10 | 34.255 | −24.503 | 67.495 | 1.00 | 70.76 |
| 4104 | CD | GLN | D | 10 | 34.942 | −25.365 | 66.456 | 1.00 | 82.40 |
| 4105 | OE1 | GLN | D | 10 | 35.249 | −26.535 | 66.706 | 1.00 | 85.25 |
| 4106 | NE2 | GLN | D | 10 | 35.192 | −24.791 | 65.282 | 1.00 | 83.74 |
| 4107 | C | GLN | D | 10 | 30.897 | −24.529 | 69.271 | 1.00 | 63.68 |
| 4108 | O | GLN | D | 10 | 30.002 | −23.797 | 68.811 | 1.00 | 68.67 |
| 4109 | N | GLU | D | 11 | 30.638 | −25.617 | 69.985 | 1.00 | 67.43 |
| 4110 | CA | GLU | D | 11 | 29.249 | −25.956 | 70.260 | 1.00 | 72.87 |
| 4111 | CB | GLU | D | 11 | 28.361 | −25.659 | 69.036 | 1.00 | 72.17 |
| 4112 | CG | GLU | D | 11 | 28.363 | −26.723 | 67.979 | 1.00 | 77.23 |
| 4113 | CD | GLU | D | 11 | 27.039 | −26.794 | 67.283 | 1.00 | 77.48 |
| 4114 | OE1 | GLU | D | 11 | 26.074 | −26.166 | 67.778 | 1.00 | 82.39 |
| 4115 | OE2 | GLU | D | 11 | 26.951 | −27.477 | 66.243 | 1.00 | 84.23 |
| 4116 | C | GLU | D | 11 | 29.037 | −27.402 | 70.720 | 1.00 | 75.56 |
| 4117 | O | GLU | D | 11 | 28.474 | −28.237 | 69.996 | 1.00 | 78.13 |
| 4118 | N | GLY | D | 12 | 29.481 | −27.709 | 71.933 | 1.00 | 74.76 |
| 4119 | CA | GLY | D | 12 | 29.276 | −29.057 | 72.415 | 1.00 | 76.84 |
| 4120 | C | GLY | D | 12 | 30.267 | −29.492 | 73.451 | 1.00 | 79.67 |
| 4121 | O | GLY | D | 12 | 31.471 | −29.337 | 73.263 | 1.00 | 81.38 |
| 4122 | N | ALA | D | 13 | 29.748 | −30.039 | 74.550 | 1.00 | 81.59 |
| 4123 | CA | ALA | D | 13 | 30.594 | −30.530 | 75.638 | 1.00 | 82.83 |
| 4124 | CB | ALA | D | 13 | 29.784 | −30.691 | 76.915 | 1.00 | 78.50 |
| 4125 | C | ALA | D | 13 | 31.237 | −31.872 | 75.245 | 1.00 | 83.89 |
| 4126 | O | ALA | D | 13 | 30.559 | −32.921 | 75.372 | 1.00 | 84.11 |
| 4127 | OT | ALA | D | 13 | 32.413 | −31.859 | 74.786 | 1.00 | 85.25 |
| 4128 | | ALA | D | 13 | | | | | |
| 4129 | C1 | MON | E | 1 | 62.540 | 2.237 | 92.135 | 1.00 | 23.02 |
| 4130 | C2 | MON | E | 1 | 62.923 | 0.723 | 92.177 | 1.00 | 28.08 |
| 4131 | C3 | MON | E | 1 | 61.083 | 2.417 | 92.743 | 1.00 | 26.18 |
| 4132 | C4 | MON | E | 1 | 63.558 | 3.145 | 92.927 | 1.00 | 24.77 |
| 4133 | C5 | MON | E | 1 | 61.958 | −0.187 | 91.323 | 1.00 | 26.56 |
| 4134 | C6 | MON | E | 1 | 64.314 | 0.292 | 91.658 | 1.00 | 30.49 |
| 4135 | C7 | MON | E | 1 | 60.644 | 3.957 | 92.790 | 1.00 | 27.34 |
| 4136 | C8 | MON | E | 1 | 60.038 | 1.470 | 92.014 | 1.00 | 19.55 |
| 4137 | C9 | MON | E | 1 | 63.044 | 4.480 | 93.489 | 1.00 | 36.10 |
| 4138 | C10 | MON | E | 1 | 62.683 | −1.560 | 91.586 | 1.00 | 24.95 |
| 4139 | C11 | MON | E | 1 | 60.537 | −0.009 | 92.002 | 1.00 | 29.51 |
| 4140 | C12 | MON | E | 1 | 61.841 | 0.186 | 89.786 | 1.00 | 16.20 |
| 4141 | C13 | MON | E | 1 | 64.175 | −1.229 | 91.419 | 1.00 | 18.12 |
| 4142 | C14 | MON | E | 1 | 61.755 | 4.860 | 93.463 | 1.00 | 30.42 |
| 4143 | C15 | MON | E | 1 | 59.373 | 4.137 | 93.699 | 1.00 | 30.42 |
| 4144 | C16 | MON | E | 1 | 60.385 | 4.442 | 91.315 | 1.00 | 24.14 |
| 4145 | C17 | MON | E | 1 | 62.229 | −2.860 | 90.768 | 1.00 | 29.22 |
| 4146 | C18 | MON | E | 1 | 61.404 | 6.156 | 94.244 | 1.00 | 35.00 |
| 4147 | C19 | MON | E | 1 | 58.862 | 5.583 | 93.936 | 1.00 | 46.46 |
| 4148 | C20 | MON | E | 1 | 62.844 | −4.206 | 91.248 | 1.00 | 36.67 |
| 4149 | C21 | MON | E | 1 | 60.689 | −3.060 | 90.528 | 1.00 | 25.16 |
| 4150 | C22 | MON | E | 1 | 59.966 | 6.678 | 94.082 | 1.00 | 52.71 |
| 4151 | C23 | MON | E | 1 | 61.934 | −5.259 | 91.890 | 1.00 | 28.32 |
| 4152 | O24 | MON | E | 1 | 59.955 | 7.475 | 92.901 | 1.00 | 85.00 |
| 4153 | C25 | MON | E | 1 | 62.616 | −6.595 | 92.271 | 1.00 | 33.33 |
| 4154 | C26 | MON | E | 1 | 64.049 | −6.802 | 92.694 | 1.00 | 31.56 |
| 4155 | O27 | MON | E | 1 | 62.870 | −7.033 | 93.557 | 1.00 | 34.02 |
| 4156 | C28 | MON | E | 1 | 65.035 | −5.610 | 92.962 | 1.00 | 32.42 |
| 4157 | C29 | MON | E | 1 | 64.668 | −8.091 | 92.254 | 1.00 | 25.44 |
| 4158 | | MON | E | 1 | | | | | |
| 4159 | C1 | MON | F | 1 | 33.143 | −27.208 | 92.268 | 1.00 | 31.39 |
| 4160 | C2 | MON | F | 1 | 34.685 | −27.478 | 92.340 | 1.00 | 33.41 |
| 4161 | C3 | MON | F | 1 | 32.909 | −25.745 | 91.643 | 1.00 | 34.01 |
| 4162 | C4 | MON | F | 1 | 32.368 | −28.285 | 91.413 | 1.00 | 28.68 |
| 4163 | C5 | MON | F | 1 | 35.449 | −26.445 | 93.275 | 1.00 | 31.71 |
| 4164 | C6 | MON | F | 1 | 35.179 | −28.845 | 92.873 | 1.00 | 30.43 |
| 4165 | C7 | MON | F | 1 | 31.352 | −25.416 | 91.473 | 1.00 | 29.98 |
| 4166 | C8 | MON | F | 1 | 33.719 | −24.625 | 92.441 | 1.00 | 32.49 |
| 4167 | C9 | MON | F | 1 | 31.055 | −27.856 | 90.739 | 1.00 | 30.66 |
| 4168 | C10 | MON | F | 1 | 36.887 | −27.086 | 93.091 | 1.00 | 25.29 |
| 4169 | C11 | MON | F | 1 | 35.231 | −25.010 | 92.612 | 1.00 | 30.79 |
| 4170 | C12 | MON | F | 1 | 34.930 | −26.361 | 94.778 | 1.00 | 29.42 |
| 4171 | C13 | MON | F | 1 | 36.664 | −28.611 | 93.183 | 1.00 | 24.46 |
| 4172 | C14 | MON | F | 1 | 30.589 | −26.596 | 90.731 | 1.00 | 26.79 |
| 4173 | C15 | MON | F | 1 | 31.148 | −24.136 | 90.567 | 1.00 | 26.84 |
| 4174 | C16 | MON | F | 1 | 30.744 | −25.211 | 92.903 | 1.00 | 20.76 |
| 4175 | C17 | MON | F | 1 | 38.081 | −26.548 | 93.991 | 1.00 | 27.49 |
| 4176 | C18 | MON | F | 1 | 29.330 | −26.345 | 89.865 | 1.00 | 32.12 |
| 4177 | C19 | MON | F | 1 | 29.688 | −23.746 | 90.220 | 1.00 | 32.60 |
| 4178 | C20 | MON | F | 1 | 39.496 | −27.164 | 93.664 | 1.00 | 22.54 |
| 4179 | C21 | MON | F | 1 | 38.180 | −24.968 | 94.088 | 1.00 | 16.40 |
| 4180 | C22 | MON | F | 1 | 28.700 | −24.946 | 89.952 | 1.00 | 28.64 |
| 4181 | C23 | MON | F | 1 | 40.186 | −26.571 | 92.450 | 1.00 | 19.64 |
| 4182 | O24 | MON | F | 1 | 27.766 | −25.016 | 91.017 | 1.00 | 67.54 |
| 4183 | C25 | MON | F | 1 | 41.575 | −27.102 | 92.134 | 1.00 | 14.61 |
| 4184 | C26 | MON | F | 1 | 41.899 | −28.527 | 91.780 | 1.00 | 23.10 |
| 4185 | O27 | MON | F | 1 | 42.005 | −27.366 | 90.863 | 1.00 | 30.73 |
| 4186 | C28 | MON | F | 1 | 40.778 | −29.602 | 91.579 | 1.00 | 21.29 |
| 4187 | C29 | MON | F | 1 | 43.302 | −29.021 | 92.054 | 1.00 | 32.60 |
| 4188 | | MON | F | 1 | | | | | |
| 4189 | O | HOH | W | 1 | 28.934 | −19.880 | 85.585 | 1.00 | 12.31 |
| 4190 | O | HOH | W | 2 | 55.303 | 6.531 | 98.855 | 1.00 | 12.91 |
| 4191 | O | HOH | W | 3 | 48.804 | −3.531 | 102.603 | 1.00 | 16.12 |
| 4192 | O | HOH | W | 4 | 27.154 | 4.730 | 78.611 | 1.00 | 11.66 |
| 4193 | O | HOH | W | 5 | 58.488 | 4.566 | 97.443 | 1.00 | 15.31 |
| 4194 | O | HOH | W | 6 | 40.150 | −3.783 | 102.943 | 1.00 | 16.64 |
| 4195 | O | HOH | W | 7 | 38.828 | −13.399 | 81.631 | 1.00 | 26.36 |
| 4196 | O | HOH | W | 8 | 40.894 | −5.696 | 85.588 | 1.00 | 23.67 |
| 4197 | O | HOH | W | 9 | 21.066 | −19.167 | 91.749 | 1.00 | 23.16 |
| 4198 | O | HOH | W | 10 | 23.739 | −13.477 | 73.847 | 1.00 | 27.83 |
| 4199 | O | HOH | W | 11 | 45.315 | −13.580 | 85.785 | 1.00 | 17.40 |
| 4200 | O | HOH | W | 12 | 42.829 | −3.310 | 82.124 | 1.00 | 13.24 |
| 4201 | O | HOH | W | 13 | 25.909 | −21.766 | 87.993 | 1.00 | 30.21 |
| 4202 | O | HOH | W | 14 | 43.284 | −12.942 | 99.122 | 1.00 | 34.84 |
| 4203 | O | HOH | W | 15 | 55.638 | −6.539 | 98.489 | 1.00 | 18.17 |
| 4204 | O | HOH | W | 16 | 17.863 | −15.351 | 90.350 | 1.00 | 35.08 |
| 4205 | O | HOH | W | 17 | 39.261 | 18.348 | 90.387 | 1.00 | 22.46 |
| 4206 | O | HOH | W | 18 | 60.616 | −28.385 | 89.919 | 1.00 | 15.85 |
| 4207 | O | HOH | W | 19 | 61.230 | 9.208 | 103.371 | 1.00 | 16.60 |
| 4208 | O | HOH | W | 20 | 50.860 | −42.107 | 89.699 | 1.00 | 17.48 |
| 4209 | O | HOH | W | 21 | 46.189 | 15.061 | 85.809 | 1.00 | 24.53 |
| 4210 | O | HOH | W | 22 | 52.856 | 8.642 | 98.253 | 1.00 | 36.93 |
| 4211 | O | HOH | W | 23 | 69.585 | 9.042 | 98.729 | 1.00 | 12.63 |
| 4212 | O | HOH | W | 24 | 24.849 | −16.489 | 97.853 | 1.00 | 17.98 |
| 4213 | O | HOH | W | 25 | 16.214 | −18.924 | 82.110 | 1.00 | 23.23 |
| 4214 | O | HOH | W | 26 | 23.966 | −19.961 | 100.138 | 1.00 | 18.47 |
| 4215 | O | HOH | W | 27 | 30.621 | 2.102 | 77.894 | 1.00 | 26.09 |
| 4216 | O | HOH | W | 28 | 46.695 | −34.723 | 97.299 | 1.00 | 31.88 |
| 4217 | O | HOH | W | 29 | 73.329 | 0.091 | 101.018 | 1.00 | 25.82 |
| 4218 | O | HOH | W | 30 | 57.802 | 11.513 | 84.158 | 1.00 | 28.34 |
| 4219 | O | HOH | W | 31 | 46.580 | −8.461 | 78.008 | 1.00 | 12.33 |
| 4220 | O | HOH | W | 32 | 34.842 | −5.244 | 107.956 | 1.00 | 31.46 |
| 4221 | O | HOH | W | 33 | 20.487 | 0.000 | 73.780 | 1.00 | 24.32 |
| 4222 | O | HOH | W | 34 | 36.203 | 1.678 | 77.127 | 1.00 | 32.71 |
| 4223 | O | HOH | W | 35 | 44.110 | −31.439 | 104.995 | 1.00 | 33.24 |
| 4224 | O | HOH | W | 36 | 34.481 | −11.772 | 72.134 | 1.00 | 70.97 |
| 4225 | O | HOH | W | 37 | 64.593 | 14.069 | 94.531 | 1.00 | 12.19 |
| 4226 | O | HOH | W | 38 | 40.360 | 0.619 | 76.551 | 1.00 | 23.37 |
| 4227 | O | HOH | W | 39 | 41.537 | 15.302 | 81.147 | 1.00 | 19.35 |
| 4228 | O | HOH | W | 40 | 43.053 | 13.888 | 83.764 | 1.00 | 17.50 |
| 4229 | O | HOH | W | 41 | 55.008 | 13.302 | 94.632 | 1.00 | 36.73 |
| 4230 | O | HOH | W | 42 | 48.001 | −20.353 | 68.412 | 1.00 | 20.59 |
| 4231 | O | HOH | W | 43 | 16.865 | 1.283 | 76.436 | 1.00 | 27.34 |
| 4232 | O | HOH | W | 44 | 66.328 | −9.830 | 75.869 | 1.00 | 18.16 |
| 4233 | O | HOH | W | 45 | 19.997 | 1.552 | 101.776 | 1.00 | 22.89 |
| 4234 | O | HOH | W | 46 | 46.772 | 17.189 | 93.223 | 1.00 | 31.59 |
| 4235 | O | HOH | W | 47 | 14.350 | −13.006 | 73.032 | 1.00 | 41.74 |
| 4236 | O | HOH | W | 48 | 79.024 | 1.593 | 96.331 | 1.00 | 28.42 |
| 4237 | O | HOH | W | 49 | 49.770 | 19.140 | 94.124 | 1.00 | 32.22 |
| 4238 | O | HOH | W | 50 | 66.156 | 13.292 | 84.665 | 1.00 | 19.89 |
| 4239 | O | HOH | W | 51 | 51.382 | −9.782 | 104.986 | 1.00 | 28.61 |
| 4240 | O | HOH | W | 52 | 58.197 | −17.427 | 114.144 | 1.00 | 25.62 |
| 4241 | O | HOH | W | 53 | 55.351 | 14.653 | 106.539 | 1.00 | 38.93 |
| 4242 | O | HOH | W | 54 | 48.609 | −32.570 | 106.727 | 1.00 | 20.34 |
| 4243 | O | HOH | W | 55 | 61.782 | 4.900 | 107.127 | 1.00 | 40.04 |
| 4244 | O | HOH | W | 56 | 74.497 | −5.736 | 100.029 | 1.00 | 30.53 |
| 4245 | O | HOH | W | 57 | 22.456 | −28.095 | 102.555 | 1.00 | 30.31 |
| 4246 | O | HOH | W | 58 | 20.657 | −20.305 | 78.494 | 1.00 | 40.51 |
| 4247 | O | HOH | W | 59 | 25.060 | −20.535 | 73.154 | 1.00 | 22.40 |
| 4248 | O | HOH | W | 60 | 53.571 | −9.609 | 114.984 | 1.00 | 31.88 |
| 4249 | O | HOH | W | 61 | 69.559 | −11.740 | 86.572 | 1.00 | 31.42 |
| 4250 | O | HOH | W | 62 | 51.818 | 2.263 | 74.063 | 1.00 | 29.20 |
| 4251 | O | HOH | W | 63 | 47.012 | −20.795 | 71.045 | 1.00 | 27.78 |
| 4252 | O | HOH | W | 64 | 38.871 | −10.924 | 69.744 | 1.00 | 35.12 |
| 4253 | O | HOH | W | 65 | 52.462 | −13.956 | 98.987 | 1.00 | 27.92 |
| 4254 | O | HOH | W | 66 | 23.969 | −22.605 | 99.762 | 1.00 | 32.94 |
| 4255 | O | HOH | W | 67 | 69.709 | 14.988 | 85.155 | 1.00 | 31.37 |
| 4256 | O | HOH | W | 68 | 33.026 | −35.022 | 84.323 | 1.00 | 31.30 |
| 4257 | O | HOH | W | 69 | 16.576 | −6.819 | 87.543 | 1.00 | 31.40 |
| 4258 | O | HOH | W | 70 | 65.039 | −10.371 | 114.303 | 1.00 | 32.85 |
| 4259 | O | HOH | W | 71 | 22.767 | −19.389 | 87.881 | 1.00 | 32.56 |
| 4260 | O | HOH | W | 72 | 43.815 | −10.534 | 105.710 | 1.00 | 28.31 |
| 4261 | O | HOH | W | 73 | 45.987 | 13.056 | 105.640 | 1.00 | 31.16 |
| 4262 | O | HOH | W | 74 | 47.274 | −13.091 | 87.837 | 1.00 | 32.00 |
| 4263 | O | HOH | W | 75 | 27.058 | −15.700 | 98.366 | 1.00 | 32.70 |
| 4264 | O | HOH | W | 76 | 53.257 | 20.711 | 104.933 | 1.00 | 29.91 |
| 4265 | O | HOH | W | 77 | 41.043 | −5.268 | 98.662 | 1.00 | 34.17 |
| 4266 | O | HOH | W | 78 | 44.789 | −1.809 | 84.049 | 1.00 | 29.68 |
| 4267 | O | HOH | W | 79 | 41.310 | −18.434 | 108.680 | 1.00 | 30.78 |
| 4268 | O | HOH | W | 80 | 38.207 | 11.844 | 105.526 | 1.00 | 32.08 |
| 4269 | O | HOH | W | 81 | 75.986 | −0.830 | 81.601 | 1.00 | 34.96 |
| 4270 | O | HOH | W | 82 | 58.059 | −18.316 | 88.869 | 1.00 | 33.11 |
| 4271 | O | HOH | W | 83 | 56.683 | −14.479 | 100.385 | 1.00 | 27.89 |
| 4272 | O | HOH | W | 84 | 36.703 | −25.555 | 86.528 | 1.00 | 36.34 |
| 4273 | O | HOH | W | 85 | 23.801 | −15.167 | 88.443 | 1.00 | 37.39 |
| 4274 | O | HOH | W | 86 | 42.474 | 18.828 | 97.026 | 1.00 | 31.16 |
| 4275 | O | HOH | W | 87 | 48.669 | −7.536 | 85.188 | 1.00 | 28.43 |
| 4276 | O | HOH | W | 88 | 21.591 | 0.859 | 78.322 | 1.00 | 31.94 |
| 4277 | O | HOH | W | 89 | 39.296 | −33.752 | 80.236 | 1.00 | 30.91 |
| 4278 | O | HOH | W | 90 | 47.239 | −3.382 | 83.812 | 1.00 | 33.55 |
| 4279 | O | HOH | W | 91 | 67.185 | −20.200 | 93.278 | 1.00 | 38.18 |
| 4280 | O | HOH | W | 92 | 35.195 | 19.669 | 96.797 | 1.00 | 35.99 |
| 4281 | O | HOH | W | 93 | 49.803 | −20.688 | 69.737 | 1.00 | 30.68 |
| 4282 | O | HOH | W | 94 | 64.688 | −13.968 | 95.519 | 1.00 | 31.91 |
| 4283 | O | HOH | W | 95 | 79.308 | 4.287 | 82.277 | 1.00 | 36.78 |
| 4284 | O | HOH | W | 96 | 52.001 | 10.689 | 86.394 | 1.00 | 30.39 |
| 4285 | O | HOH | W | 97 | 62.214 | 9.705 | 94.059 | 1.00 | 31.68 |
| 4286 | O | HOH | W | 98 | 67.772 | −13.671 | 78.429 | 1.00 | 32.38 |
| 4287 | O | HOH | W | 99 | 29.988 | −3.122 | 102.391 | 1.00 | 35.15 |
| 4288 | O | HOH | W | 100 | 47.533 | −20.550 | 105.577 | 1.00 | 32.36 |
| 4289 | O | HOH | W | 101 | 28.468 | −35.314 | 99.506 | 1.00 | 31.25 |
| 4290 | | HOH | W | 101 | | | | | |

TABLE 3
ATOMIC COORDINATE DATA FOR CRYSTALLIZED HUMAN LXR IN
COMPLEX WITH T317

| | ATOM | | | | | | | | |
|---|---|---|---|---|---|---|---|---|---|
| ATOM | TYPE | RESIDUE | PROTEIN # | # | X | Y | Z | OCC | B |
| 1 | N | GLN | A | 219 | 55.096 | 66.212 | 96.800 | 1.00 | 85.67 |
| 2 | CA | GLN | A | 219 | 54.529 | 67.185 | 95.712 | 1.00 | 82.61 |
| 3 | CB | GLN | A | 219 | 54.350 | 66.509 | 94.375 | 1.00 | 83.27 |
| 4 | CG | GLN | A | 219 | 54.180 | 67.585 | 93.195 | 1.00 | 85.89 |
| 5 | CD | GLN | A | 219 | 54.278 | 66.783 | 91.923 | 1.00 | 95.95 |
| 6 | OE1 | GLN | A | 219 | 54.336 | 65.475 | 91.973 | 1.00 | 103.23 |
| 7 | NE2 | GLN | A | 219 | 54.359 | 67.414 | 90.809 | 1.00 | 89.35 |
| 8 | C | GLN | A | 219 | 53.058 | 67.823 | 96.033 | 1.00 | 82.67 |
| 9 | O | GLN | A | 219 | 52.107 | 67.093 | 96.511 | 1.00 | 79.24 |
| 10 | N | LEU | A | 220 | 52.898 | 69.094 | 95.693 | 1.00 | 79.24 |
| 11 | CA | LEU | A | 220 | 51.820 | 69.917 | 96.065 | 1.00 | 79.91 |
| 12 | CB | LEU | A | 220 | 51.158 | 69.388 | 97.282 | 1.00 | 80.64 |
| 13 | CG | LEU | A | 220 | 49.656 | 69.547 | 97.370 | 1.00 | 84.65 |
| 14 | CD1 | LEU | A | 220 | 48.849 | 69.361 | 96.088 | 1.00 | 79.69 |
| 15 | CD2 | LEU | A | 220 | 49.117 | 68.499 | 98.222 | 1.00 | 77.73 |
| 16 | C | LEU | A | 220 | 52.364 | 71.275 | 96.415 | 1.00 | 78.44 |
| 17 | O | LEU | A | 220 | 52.933 | 71.867 | 95.640 | 1.00 | 79.65 |
| 18 | N | THR | A | 221 | 52.200 | 71.813 | 97.579 | 1.00 | 80.24 |
| 19 | CA | THR | A | 221 | 52.798 | 73.106 | 98.039 | 1.00 | 74.95 |
| 20 | CB | THR | A | 221 | 52.210 | 74.371 | 97.456 | 1.00 | 75.35 |
| 21 | OG1 | THR | A | 221 | 50.852 | 74.573 | 97.853 | 1.00 | 71.53 |
| 22 | CG2 | THR | A | 221 | 52.375 | 74.405 | 96.081 | 1.00 | 75.02 |
| 23 | C | THR | A | 221 | 52.439 | 73.251 | 99.482 | 1.00 | 73.49 |
| 24 | O | THR | A | 221 | 51.715 | 72.620 | 99.956 | 1.00 | 73.98 |
| 25 | N | ALA | A | 222 | 53.115 | 74.057 | 100.113 | 1.00 | 74.29 |
| 26 | CA | ALA | A | 222 | 53.083 | 74.324 | 101.434 | 1.00 | 73.25 |
| 27 | CB | ALA | A | 222 | 54.004 | 75.789 | 101.677 | 1.00 | 74.00 |
| 28 | C | ALA | A | 222 | 51.715 | 74.696 | 101.469 | 1.00 | 73.19 |
| 29 | O | ALA | A | 222 | 50.917 | 74.126 | 102.291 | 1.00 | 77.41 |
| 30 | N | ALA | A | 223 | 51.341 | 75.731 | 100.749 | 1.00 | 69.56 |
| 31 | CA | ALA | A | 223 | 50.011 | 76.343 | 100.979 | 1.00 | 67.22 |
| 32 | CB | ALA | A | 223 | 49.942 | 77.645 | 100.030 | 1.00 | 67.04 |
| 33 | C | ALA | A | 223 | 48.810 | 75.386 | 100.549 | 1.00 | 62.36 |
| 34 | O | ALA | A | 223 | 48.033 | 74.990 | 101.401 | 1.00 | 72.14 |
| 35 | N | GLN | A | 224 | 48.818 | 74.892 | 99.343 | 1.00 | 57.24 |
| 36 | CA | GLN | A | 224 | 47.951 | 73.815 | 99.012 | 1.00 | 54.17 |
| 37 | CB | GLN | A | 224 | 48.281 | 73.169 | 97.735 | 1.00 | 51.05 |
| 38 | CG | GLN | A | 224 | 48.294 | 74.054 | 96.470 | 1.00 | 53.95 |
| 39 | CD | GLN | A | 224 | 48.784 | 73.339 | 95.226 | 1.00 | 54.65 |
| 40 | OE1 | GLN | A | 224 | 49.714 | 72.333 | 95.279 | 1.00 | 54.13 |
| 41 | NE2 | GLN | A | 224 | 48.218 | 73.688 | 94.078 | 1.00 | 51.29 |
| 42 | C | GLN | A | 224 | 47.618 | 72.858 | 100.028 | 1.00 | 49.42 |
| 43 | O | GLN | A | 224 | 46.548 | 72.535 | 100.328 | 1.00 | 49.40 |
| 44 | N | GLU | A | 225 | 48.731 | 72.445 | 100.710 | 1.00 | 55.66 |
| 45 | CA | GLU | A | 225 | 48.574 | 71.451 | 101.769 | 1.00 | 55.71 |
| 46 | CB | GLU | A | 225 | 49.917 | 70.803 | 102.206 | 1.00 | 50.35 |
| 47 | CG | GLU | A | 225 | 49.846 | 69.389 | 102.481 | 1.00 | 61.55 |
| 48 | CD | GLU | A | 225 | 51.273 | 68.720 | 102.141 | 1.00 | 71.52 |
| 49 | OE1 | GLU | A | 225 | 52.220 | 69.379 | 101.660 | 1.00 | 64.80 |
| 50 | OE2 | GLU | A | 225 | 51.344 | 67.494 | 102.382 | 1.00 | 73.78 |
| 51 | C | GLU | A | 225 | 47.701 | 71.907 | 102.867 | 1.00 | 52.09 |
| 52 | O | GLU | A | 225 | 46.781 | 71.164 | 103.301 | 1.00 | 54.24 |
| 53 | N | LEU | A | 226 | 48.006 | 73.078 | 103.293 | 1.00 | 55.30 |
| 54 | CA | LEU | A | 226 | 47.256 | 73.687 | 104.375 | 1.00 | 63.12 |
| 55 | CB | LEU | A | 226 | 48.088 | 74.929 | 105.072 | 1.00 | 64.83 |
| 56 | CG | LEU | A | 226 | 47.422 | 75.271 | 106.441 | 1.00 | 60.16 |
| 57 | CD1 | LEU | A | 226 | 48.170 | 74.146 | 107.428 | 1.00 | 71.82 |
| 58 | CD2 | LEU | A | 226 | 47.731 | 76.481 | 106.932 | 1.00 | 69.43 |
| 59 | C | LEU | A | 226 | 45.854 | 74.173 | 103.963 | 1.00 | 53.65 |
| 60 | O | LEU | A | 226 | 44.871 | 74.165 | 104.661 | 1.00 | 56.35 |
| 61 | N | MET | A | 227 | 45.821 | 74.622 | 102.833 | 1.00 | 51.57 |
| 62 | CA | MET | A | 227 | 44.494 | 75.143 | 102.213 | 1.00 | 46.46 |
| 63 | CB | MET | A | 227 | 44.707 | 75.666 | 100.839 | 1.00 | 40.11 |
| 64 | CG | MET | A | 227 | 43.351 | 75.935 | 100.159 | 1.00 | 57.58 |
| 65 | SD | MET | A | 227 | 43.040 | 74.836 | 98.642 | 1.00 | 55.13 |
| 66 | CE | MET | A | 227 | 43.443 | 76.156 | 97.164 | 1.00 | 43.97 |
| 67 | C | MET | A | 227 | 43.500 | 73.806 | 102.197 | 1.00 | 46.27 |
| 68 | O | MET | A | 227 | 42.314 | 73.931 | 102.564 | 1.00 | 44.18 |
| 69 | N | ILE | A | 228 | 44.026 | 72.642 | 101.809 | 1.00 | 41.32 |
| 70 | CA | ILE | A | 228 | 43.169 | 71.503 | 101.779 | 1.00 | 46.75 |
| 71 | CB | ILE | A | 228 | 43.899 | 70.245 | 101.070 | 1.00 | 47.57 |
| 72 | CG1 | ILE | A | 228 | 43.966 | 70.644 | 99.538 | 1.00 | 48.88 |
| 73 | CD1 | ILE | A | 228 | 44.649 | 69.699 | 98.798 | 1.00 | 35.69 |
| 74 | CG2 | ILE | A | 228 | 43.088 | 69.008 | 101.220 | 1.00 | 40.13 |
| 75 | C | ILE | A | 228 | 42.873 | 71.002 | 103.186 | 1.00 | 46.79 |
| 76 | O | ILE | A | 228 | 41.711 | 70.652 | 103.505 | 1.00 | 51.05 |
| 77 | N | GLN | A | 229 | 43.808 | 71.198 | 104.076 | 1.00 | 48.96 |
| 78 | CA | GLN | A | 229 | 43.612 | 70.846 | 105.505 | 1.00 | 48.59 |
| 79 | CB | GLN | A | 229 | 45.088 | 71.164 | 106.354 | 1.00 | 53.21 |
| 80 | CG | GLN | A | 229 | 45.941 | 69.894 | 106.483 | 1.00 | 49.89 |
| 81 | CD | GLN | A | 229 | 47.568 | 70.062 | 106.489 | 1.00 | 50.76 |
| 82 | OE1 | GLN | A | 229 | 48.046 | 71.110 | 106.750 | 1.00 | 50.99 |
| 83 | NE2 | GLN | A | 229 | 48.206 | 69.215 | 105.818 | 1.00 | 45.36 |
| 84 | C | GLN | A | 229 | 42.663 | 71.845 | 106.000 | 1.00 | 45.24 |
| 85 | O | GLN | A | 229 | 41.754 | 71.505 | 106.682 | 1.00 | 49.82 |
| 86 | N | GLN | A | 230 | 42.798 | 73.073 | 105.581 | 1.00 | 45.98 |
| 87 | CA | GLN | A | 230 | 41.832 | 74.124 | 105.936 | 1.00 | 47.46 |
| 88 | CB | GLN | A | 230 | 42.321 | 75.354 | 105.158 | 1.00 | 54.17 |
| 89 | CG | GLN | A | 230 | 41.872 | 76.768 | 105.720 | 1.00 | 56.74 |
| 90 | CD | GLN | A | 230 | 43.023 | 77.796 | 105.391 | 1.00 | 62.88 |
| 91 | OE1 | GLN | A | 230 | 44.232 | 77.845 | 106.181 | 1.00 | 62.21 |
| 92 | NE2 | GLN | A | 230 | 42.870 | 78.490 | 104.372 | 1.00 | 49.94 |
| 93 | C | GLN | A | 230 | 40.383 | 73.767 | 105.479 | 1.00 | 45.84 |
| 94 | O | GLN | A | 230 | 39.482 | 73.992 | 106.224 | 1.00 | 50.05 |
| 95 | N | LEU | A | 231 | 40.143 | 73.388 | 104.196 | 1.00 | 40.18 |
| 96 | CA | LEU | A | 231 | 38.803 | 73.117 | 103.696 | 1.00 | 36.97 |
| 97 | CB | LEU | A | 231 | 38.847 | 72.671 | 102.249 | 1.00 | 34.49 |
| 98 | CG | LEU | A | 231 | 39.003 | 73.787 | 101.287 | 1.00 | 39.49 |
| 99 | CD1 | LEU | A | 231 | 39.085 | 73.333 | 99.955 | 1.00 | 32.02 |
| 100 | CD2 | LEU | A | 231 | 37.892 | 74.819 | 101.569 | 1.00 | 38.12 |
| 101 | C | LEU | A | 231 | 38.331 | 71.856 | 104.456 | 1.00 | 39.71 |
| 102 | O | LEU | A | 231 | 37.235 | 71.863 | 104.897 | 1.00 | 36.04 |
| 103 | N | VAL | A | 232 | 39.238 | 70.801 | 104.575 | 1.00 | 37.04 |
| 104 | CA | VAL | A | 232 | 38.802 | 69.651 | 105.266 | 1.00 | 39.92 |
| 105 | CB | VAL | A | 232 | 39.767 | 68.563 | 105.281 | 1.00 | 41.89 |
| 106 | CG1 | VAL | A | 232 | 39.460 | 67.424 | 106.242 | 1.00 | 35.24 |
| 107 | CG2 | VAL | A | 232 | 39.789 | 67.924 | 103.872 | 1.00 | 34.57 |
| 108 | C | VAL | A | 232 | 38.425 | 69.942 | 106.720 | 1.00 | 40.19 |
| 109 | O | VAL | A | 232 | 37.240 | 69.481 | 107.193 | 1.00 | 37.03 |
| 110 | N | ALA | A | 233 | 39.056 | 70.859 | 107.305 | 1.00 | 39.26 |
| 111 | CA | ALA | A | 233 | 38.716 | 71.119 | 108.791 | 1.00 | 40.47 |
| 112 | CB | ALA | A | 233 | 40.043 | 71.949 | 109.543 | 1.00 | 42.60 |
| 113 | C | ALA | A | 233 | 37.408 | 71.881 | 108.792 | 1.00 | 43.83 |
| 114 | O | ALA | A | 233 | 36.576 | 71.750 | 109.707 | 1.00 | 42.76 |
| 115 | N | ALA | A | 234 | 37.222 | 72.791 | 107.913 | 1.00 | 41.01 |
| 116 | CA | ALA | A | 234 | 35.966 | 73.476 | 107.940 | 1.00 | 43.14 |
| 117 | CB | ALA | A | 234 | 35.940 | 74.622 | 106.955 | 1.00 | 36.75 |
| 118 | C | ALA | A | 234 | 34.904 | 72.584 | 107.775 | 1.00 | 43.94 |
| 119 | O | ALA | A | 234 | 33.856 | 72.638 | 108.338 | 1.00 | 46.99 |
| 120 | N | GLN | A | 235 | 35.068 | 71.667 | 106.881 | 1.00 | 47.45 |
| 121 | CA | GLN | A | 235 | 34.069 | 70.721 | 106.554 | 1.00 | 48.08 |
| 122 | CB | GLN | A | 235 | 34.546 | 69.825 | 105.467 | 1.00 | 52.03 |
| 123 | CG | GLN | A | 235 | 33.783 | 68.617 | 105.047 | 1.00 | 53.41 |
| 124 | CD | GLN | A | 235 | 34.678 | 67.450 | 104.499 | 1.00 | 52.47 |
| 125 | OE1 | GLN | A | 235 | 34.652 | 67.038 | 103.328 | 1.00 | 50.48 |
| 126 | NE2 | GLN | A | 235 | 35.311 | 66.792 | 105.516 | 1.00 | 54.98 |
| 127 | C | GLN | A | 235 | 33.789 | 69.936 | 107.752 | 1.00 | 53.30 |
| 128 | O | GLN | A | 235 | 32.550 | 69.602 | 107.989 | 1.00 | 55.90 |
| 129 | N | LEU | A | 236 | 34.758 | 69.511 | 108.526 | 1.00 | 50.61 |
| 130 | CA | LEU | A | 236 | 34.344 | 68.716 | 109.825 | 1.00 | 57.13 |
| 131 | CB | LEU | A | 236 | 35.603 | 68.079 | 110.441 | 1.00 | 57.67 |
| 132 | CG | LEU | A | 236 | 35.237 | 66.766 | 111.196 | 1.00 | 61.22 |
| 133 | CD1 | LEU | A | 236 | 34.524 | 65.745 | 110.294 | 1.00 | 52.82 |
| 134 | CD2 | LEU | A | 236 | 36.559 | 66.177 | 111.825 | 1.00 | 58.45 |
| 135 | C | LEU | A | 236 | 33.716 | 69.546 | 110.974 | 1.00 | 51.69 |
| 136 | O | LEU | A | 236 | 32.895 | 68.997 | 111.546 | 1.00 | 55.63 |
| 137 | N | GLN | A | 237 | 34.116 | 70.794 | 111.176 | 1.00 | 53.41 |
| 138 | CA | GLN | A | 237 | 33.573 | 71.628 | 112.172 | 1.00 | 58.99 |
| 139 | CB | GLN | A | 237 | 34.387 | 72.925 | 112.344 | 1.00 | 61.90 |
| 140 | CG | GLN | A | 237 | 34.237 | 73.820 | 113.590 | 1.00 | 70.99 |
| 141 | CD | GLN | A | 237 | 35.116 | 75.089 | 113.520 | 1.00 | 85.40 |
| 142 | OE1 | GLN | A | 237 | 36.371 | 74.899 | 113.536 | 1.00 | 89.97 |
| 143 | NE2 | GLN | A | 237 | 34.525 | 76.336 | 113.541 | 1.00 | 84.12 |
| 144 | C | GLN | A | 237 | 32.156 | 71.934 | 111.994 | 1.00 | 63.64 |
| 145 | O | GLN | A | 237 | 31.394 | 72.275 | 112.889 | 1.00 | 65.52 |
| 146 | N | CYS | A | 238 | 31.808 | 72.009 | 110.759 | 1.00 | 66.85 |
| 147 | CA | CYS | A | 238 | 30.384 | 72.242 | 110.386 | 1.00 | 70.91 |
| 148 | CB | CYS | A | 238 | 30.325 | 72.710 | 108.807 | 1.00 | 67.17 |
| 149 | SG | CYS | A | 238 | 31.327 | 74.207 | 108.665 | 1.00 | 71.62 |
| 150 | C | CYS | A | 238 | 29.541 | 70.943 | 110.485 | 1.00 | 66.87 |
| 151 | O | CYS | A | 238 | 28.518 | 71.003 | 110.575 | 1.00 | 72.18 |
| 152 | N | ASN | A | 239 | 30.100 | 69.844 | 110.273 | 1.00 | 72.55 |
| 153 | CA | ASN | A | 239 | 29.441 | 68.505 | 110.351 | 1.00 | 79.18 |
| 154 | CB | ASN | A | 239 | 30.340 | 67.296 | 109.802 | 1.00 | 77.58 |
| 155 | CG | ASN | A | 239 | 30.400 | 67.258 | 108.284 | 1.00 | 79.69 |
| 156 | OD1 | ASN | A | 239 | 31.077 | 66.423 | 107.777 | 1.00 | 83.87 |
| 157 | ND2 | ASN | A | 239 | 29.573 | 68.038 | 107.558 | 1.00 | 77.69 |
| 158 | C | ASN | A | 239 | 29.003 | 68.107 | 111.728 | 1.00 | 75.62 |
| 159 | O | ASN | A | 239 | 27.914 | 67.730 | 111.832 | 1.00 | 74.50 |
| 160 | N | LYS | A | 240 | 29.869 | 68.235 | 112.721 | 1.00 | 80.50 |
| 161 | CA | LYS | A | 240 | 29.464 | 67.794 | 114.144 | 1.00 | 86.13 |
| 162 | CB | LYS | A | 240 | 30.706 | 67.336 | 115.048 | 1.00 | 90.30 |
| 163 | CG | LYS | A | 240 | 30.231 | 66.406 | 116.357 | 1.00 | 94.45 |
| 164 | CD | LYS | A | 240 | 31.176 | 66.647 | 117.471 | 1.00 | 99.51 |
| 165 | CE | LYS | A | 240 | 32.269 | 65.606 | 117.395 | 1.00 | 114.82 |
| 166 | NZ | LYS | A | 240 | 32.562 | 65.041 | 115.858 | 1.00 | 114.06 |
| 167 | C | LYS | A | 240 | 28.644 | 68.816 | 114.771 | 1.00 | 85.69 |
| 168 | O | LYS | A | 240 | 27.964 | 68.439 | 115.796 | 1.00 | 89.68 |
| 169 | N | ALA | A | 241 | 28.677 | 70.099 | 114.193 | 1.00 | 87.24 |
| 170 | CA | ALA | A | 241 | 27.928 | 71.179 | 114.681 | 1.00 | 87.61 |
| 171 | CB | ALA | A | 241 | 28.294 | 72.335 | 114.124 | 1.00 | 88.25 |
| 172 | C | ALA | A | 241 | 26.500 | 70.745 | 114.193 | 1.00 | 94.00 |
| 173 | O | ALA | A | 241 | 25.570 | 71.490 | 114.394 | 1.00 | 96.99 |
| 174 | N | SER | A | 242 | 26.289 | 69.518 | 113.629 | 1.00 | 96.44 |
| 175 | CA | SER | A | 242 | 24.966 | 69.057 | 113.224 | 1.00 | 94.27 |
| 176 | CB | SER | A | 242 | 25.126 | 68.841 | 111.704 | 1.00 | 100.35 |
| 177 | OG | SER | A | 242 | 24.364 | 69.685 | 110.752 | 1.00 | 93.83 |
| 178 | C | SER | A | 242 | 24.649 | 67.769 | 113.864 | 1.00 | 93.07 |
| 179 | O | SER | A | 242 | 23.418 | 67.451 | 114.167 | 1.00 | 97.73 |
| 180 | N | ALA | A | 243 | 25.650 | 66.945 | 114.079 | 1.00 | 86.82 |
| 181 | CA | ALA | A | 243 | 25.374 | 65.747 | 114.932 | 1.00 | 86.98 |
| 182 | CB | ALA | A | 243 | 26.393 | 64.855 | 114.994 | 1.00 | 82.85 |
| 183 | C | ALA | A | 243 | 25.174 | 66.286 | 116.424 | 1.00 | 88.53 |
| 184 | O | ALA | A | 243 | 24.515 | 65.662 | 117.261 | 1.00 | 89.29 |
| 185 | N | SER | A | 244 | 25.886 | 67.365 | 116.766 | 1.00 | 87.28 |
| 186 | CA | SER | A | 244 | 25.885 | 67.925 | 118.137 | 1.00 | 87.90 |
| 187 | CB | SER | A | 244 | 27.031 | 68.890 | 118.287 | 1.00 | 84.27 |
| 188 | OG | SER | A | 244 | 28.220 | 68.341 | 117.980 | 1.00 | 83.58 |
| 189 | C | SER | A | 244 | 24.541 | 68.719 | 118.279 | 1.00 | 88.50 |
| 190 | O | SER | A | 244 | 24.130 | 68.910 | 119.279 | 1.00 | 88.97 |
| 191 | N | ASP | A | 245 | 23.948 | 69.122 | 117.150 | 1.00 | 88.90 |
| 192 | CA | ASP | A | 245 | 22.774 | 69.984 | 116.979 | 1.00 | 90.49 |
| 193 | CB | ASP | A | 245 | 22.831 | 71.363 | 116.239 | 1.00 | 86.09 |
| 194 | CG | ASP | A | 245 | 24.065 | 72.203 | 116.513 | 1.00 | 96.91 |
| 195 | OD1 | ASP | A | 245 | 25.050 | 71.944 | 117.405 | 1.00 | 111.52 |
| 196 | OD2 | ASP | A | 245 | 24.146 | 73.306 | 115.856 | 1.00 | 104.66 |
| 197 | C | ASP | A | 245 | 21.813 | 69.325 | 116.144 | 1.00 | 91.23 |
| 198 | O | ASP | A | 245 | 20.894 | 70.003 | 115.611 | 1.00 | 90.41 |
| 199 | N | GLN | A | 246 | 21.951 | 67.987 | 116.053 | 1.00 | 94.12 |
| 200 | CA | GLN | A | 246 | 20.851 | 67.266 | 115.352 | 1.00 | 94.06 |
| 201 | CB | GLN | A | 246 | 21.208 | 65.780 | 115.151 | 1.00 | 91.22 |
| 202 | CG | GLN | A | 246 | 19.998 | 65.123 | 114.700 | 1.00 | 87.58 |
| 203 | CD | GLN | A | 246 | 19.410 | 65.721 | 113.431 | 1.00 | 91.60 |
| 204 | OE1 | GLN | A | 246 | 20.212 | 66.018 | 112.476 | 1.00 | 98.62 |
| 205 | NE2 | GLN | A | 246 | 18.059 | 65.907 | 113.362 | 1.00 | 85.44 |
| 206 | C | GLN | A | 246 | 19.547 | 67.427 | 116.192 | 1.00 | 96.50 |
| 207 | O | GLN | A | 246 | 18.568 | 66.700 | 115.794 | 1.00 | 99.52 |
| 208 | N | PRO | A | 247 | 19.436 | 68.354 | 117.319 | 1.00 | 99.12 |
| 209 | CA | PRO | A | 247 | 18.094 | 68.308 | 117.993 | 1.00 | 101.73 |
| 210 | CB | PRO | A | 247 | 18.370 | 68.795 | 119.535 | 1.00 | 100.37 |
| 211 | CG | PRO | A | 247 | 19.989 | 69.087 | 119.532 | 1.00 | 96.35 |
| 212 | CD | PRO | A | 247 | 20.260 | 69.364 | 118.087 | 1.00 | 97.58 |
| 213 | C | PRO | A | 247 | 17.010 | 69.133 | 117.063 | 1.00 | 99.53 |
| 214 | O | PRO | A | 247 | 17.355 | 69.673 | 115.984 | 1.00 | 99.61 |
| 215 | N | LYS | A | 248 | 15.765 | 69.119 | 117.599 | 1.00 | 97.51 |
| 216 | CA | LYS | A | 248 | 14.676 | 69.814 | 117.113 | 1.00 | 91.20 |
| 217 | CB | LYS | A | 248 | 15.054 | 71.316 | 117.046 | 1.00 | 94.85 |
| 218 | CG | LYS | A | 248 | 16.592 | 71.766 | 116.943 | 1.00 | 97.98 |
| 219 | CD | LYS | A | 248 | 16.733 | 73.049 | 117.751 | 1.00 | 97.99 |
| 220 | CE | LYS | A | 248 | 16.110 | 72.909 | 119.070 | 1.00 | 86.59 |
| 221 | NZ | LYS | A | 248 | 17.446 | 72.996 | 119.902 | 1.00 | 101.33 |
| 222 | C | LYS | A | 248 | 14.035 | 69.257 | 115.840 | 1.00 | 85.99 |
| 223 | O | LYS | A | 248 | 13.563 | 70.079 | 115.076 | 1.00 | 87.81 |
| 224 | N | VAL | A | 249 | 13.806 | 67.947 | 115.695 | 1.00 | 78.52 |
| 225 | CA | VAL | A | 249 | 13.169 | 67.528 | 114.477 | 1.00 | 73.17 |
| 226 | CB | VAL | A | 249 | 14.142 | 66.871 | 113.498 | 1.00 | 73.37 |
| 227 | CG1 | VAL | A | 249 | 14.618 | 65.525 | 114.094 | 1.00 | 79.27 |
| 228 | CG2 | VAL | A | 249 | 13.499 | 66.651 | 112.317 | 1.00 | 70.29 |
| 229 | C | VAL | A | 249 | 11.941 | 66.663 | 114.641 | 1.00 | 73.94 |
| 230 | O | VAL | A | 249 | 11.781 | 65.828 | 115.543 | 1.00 | 67.47 |
| 231 | N | THR | A | 250 | 11.036 | 66.879 | 113.685 | 1.00 | 75.15 |
| 232 | CA | THR | A | 250 | 9.765 | 66.196 | 113.495 | 1.00 | 68.88 |
| 233 | CB | THR | A | 250 | 8.903 | 66.734 | 112.251 | 1.00 | 62.96 |
| 234 | OG1 | THR | A | 250 | 8.354 | 67.933 | 112.477 | 1.00 | 59.52 |
| 235 | CG2 | THR | A | 250 | 7.735 | 65.788 | 111.930 | 1.00 | 67.09 |
| 236 | C | THR | A | 250 | 10.091 | 64.666 | 113.423 | 1.00 | 71.32 |
| 237 | O | THR | A | 250 | 10.778 | 64.237 | 112.642 | 1.00 | 72.94 |
| 238 | N | PRO | A | 251 | 9.483 | 63.858 | 114.238 | 1.00 | 74.41 |
| 239 | CA | PRO | A | 251 | 9.750 | 62.512 | 114.198 | 1.00 | 72.78 |
| 240 | CB | PRO | A | 251 | 9.052 | 62.108 | 115.505 | 1.00 | 72.73 |
| 241 | CG | PRO | A | 251 | 8.954 | 63.316 | 116.364 | 1.00 | 69.09 |
| 242 | CD | PRO | A | 251 | 8.519 | 64.152 | 115.411 | 1.00 | 71.69 |
| 243 | C | PRO | A | 251 | 9.001 | 61.917 | 113.044 | 1.00 | 77.05 |
| 244 | O | PRO | A | 251 | 7.996 | 62.327 | 112.468 | 1.00 | 79.06 |
| 245 | N | TRP | A | 252 | 9.579 | 60.819 | 112.659 | 1.00 | 75.63 |
| 246 | CA | TRP | A | 252 | 9.071 | 60.047 | 111.528 | 1.00 | 76.35 |
| 247 | CB | TRP | A | 252 | 10.425 | 59.392 | 110.932 | 1.00 | 70.22 |
| 248 | CG | TRP | A | 252 | 10.341 | 58.587 | 109.881 | 1.00 | 55.93 |
| 249 | CD1 | TRP | A | 252 | 10.066 | 57.235 | 109.857 | 1.00 | 65.14 |
| 250 | NE1 | TRP | A | 252 | 10.031 | 56.722 | 108.564 | 1.00 | 61.03 |
| 251 | CE2 | TRP | A | 252 | 10.278 | 57.810 | 107.707 | 1.00 | 58.03 |
| 252 | CD2 | TRP | A | 252 | 10.462 | 58.969 | 108.498 | 1.00 | 54.59 |
| 253 | CE3 | TRP | A | 252 | 10.596 | 60.178 | 107.878 | 1.00 | 51.44 |
| 254 | CZ3 | TRP | A | 252 | 10.557 | 60.237 | 106.538 | 1.00 | 55.11 |
| 255 | CH2 | TRP | A | 252 | 10.407 | 59.050 | 105.787 | 1.00 | 57.74 |
| 256 | CZ2 | TRP | A | 252 | 10.237 | 57.844 | 106.405 | 1.00 | 57.19 |
| 257 | C | TRP | A | 252 | 8.133 | 58.878 | 112.057 | 1.00 | 78.02 |
| 258 | O | TRP | A | 252 | 8.477 | 58.241 | 113.199 | 1.00 | 80.22 |
| 259 | N | PRO | A | 253 | 7.117 | 58.530 | 111.261 | 1.00 | 79.55 |
| 260 | CA | PRO | A | 253 | 6.089 | 57.490 | 111.359 | 1.00 | 81.01 |
| 261 | CB | PRO | A | 253 | 5.541 | 57.495 | 109.968 | 1.00 | 77.67 |
| 262 | CG | PRO | A | 253 | 6.227 | 58.502 | 109.284 | 1.00 | 83.93 |
| 263 | CD | PRO | A | 253 | 6.718 | 59.510 | 110.314 | 1.00 | 82.51 |
| 264 | C | PRO | A | 253 | 6.428 | 55.966 | 111.829 | 1.00 | 88.29 |
| 265 | O | PRO | A | 253 | 7.520 | 55.359 | 111.483 | 1.00 | 94.15 |
| 266 | N | ALA | A | 254 | 5.573 | 55.304 | 112.625 | 1.00 | 89.95 |
| 267 | CA | ALA | A | 254 | 5.653 | 53.907 | 112.956 | 1.00 | 89.10 |
| 268 | CB | ALA | A | 254 | 4.547 | 53.743 | 113.813 | 1.00 | 92.19 |
| 269 | C | ALA | A | 254 | 5.330 | 53.237 | 111.636 | 1.00 | 92.55 |
| 270 | O | ALA | A | 254 | 5.413 | 51.940 | 111.550 | 1.00 | 89.38 |
| 271 | N | GLY | A | 255 | 4.897 | 54.159 | 110.611 | 1.00 | 93.89 |
| 272 | CA | GLY | A | 255 | 4.572 | 53.948 | 109.170 | 1.00 | 94.59 |
| 273 | C | GLY | A | 255 | 3.390 | 53.131 | 108.738 | 1.00 | 96.06 |
| 274 | O | GLY | A | 255 | 3.756 | 52.461 | 107.673 | 1.00 | 88.95 |
| 275 | OXT | GLY | A | 255 | 2.282 | 53.352 | 109.541 | 1.00 | 100.39 |
| 276 | N | GLN | A | 259 | −0.551 | 53.657 | 105.997 | 1.00 | 100.98 |
| 277 | CA | GLN | A | 259 | −0.692 | 54.510 | 104.638 | 1.00 | 101.82 |
| 278 | CB | GLN | A | 259 | −1.095 | 53.722 | 103.374 | 1.00 | 103.68 |
| 279 | CG | GLN | A | 259 | −0.017 | 52.868 | 102.666 | 1.00 | 102.32 |
| 280 | CD | GLN | A | 259 | −0.108 | 51.348 | 102.981 | 1.00 | 109.86 |
| 281 | OE1 | GLN | A | 259 | −0.298 | 50.854 | 104.208 | 1.00 | 111.68 |
| 282 | NE2 | GLN | A | 259 | −0.018 | 50.545 | 101.859 | 1.00 | 111.42 |
| 283 | C | GLN | A | 259 | −1.796 | 55.517 | 104.748 | 1.00 | 101.08 |
| 284 | O | GLN | A | 259 | −2.998 | 55.218 | 104.317 | 1.00 | 102.37 |
| 285 | N | SER | A | 260 | −1.436 | 56.680 | 105.356 | 1.00 | 97.79 |
| 286 | CA | SER | A | 260 | −2.368 | 57.733 | 105.632 | 1.00 | 92.33 |
| 287 | CB | SER | A | 260 | −2.273 | 57.895 | 107.119 | 1.00 | 92.61 |
| 288 | OG | SER | A | 260 | −1.724 | 59.173 | 107.506 | 1.00 | 78.41 |
| 289 | C | SER | A | 260 | −2.091 | 59.051 | 104.948 | 1.00 | 95.05 |
| 290 | O | SER | A | 260 | −1.459 | 59.094 | 103.938 | 1.00 | 99.90 |
| 291 | N | ARG | A | 261 | −2.519 | 60.156 | 105.517 | 1.00 | 94.75 |
| 292 | CA | ARG | A | 261 | −2.360 | 61.518 | 104.997 | 1.00 | 93.13 |
| 293 | CB | ARG | A | 261 | −3.737 | 62.062 | 104.724 | 1.00 | 94.35 |
| 294 | CG | ARG | A | 261 | −4.900 | 61.003 | 105.277 | 1.00 | 101.94 |
| 295 | CD | ARG | A | 261 | −6.376 | 61.721 | 105.522 | 1.00 | 101.64 |
| 296 | NE | ARG | A | 261 | −6.597 | 62.608 | 104.359 | 1.00 | 104.47 |
| 297 | CZ | ARG | A | 261 | −7.787 | 63.041 | 104.006 | 1.00 | 101.15 |
| 298 | NH1 | ARG | A | 261 | −8.781 | 62.772 | 104.792 | 1.00 | 102.77 |
| 299 | NH2 | ARG | A | 261 | −7.943 | 63.793 | 102.950 | 1.00 | 94.43 |
| 300 | C | ARG | A | 261 | −1.665 | 62.476 | 105.906 | 1.00 | 90.89 |
| 301 | O | ARG | A | 261 | −1.034 | 63.368 | 105.471 | 1.00 | 88.73 |
| 302 | N | ASP | A | 262 | −1.868 | 62.289 | 107.191 | 1.00 | 90.00 |
| 303 | CA | ASP | A | 262 | −1.239 | 63.018 | 108.247 | 1.00 | 87.96 |
| 304 | CB | ASP | A | 262 | −1.906 | 62.624 | 109.569 | 1.00 | 90.25 |
| 305 | CG | ASP | A | 262 | −1.449 | 63.451 | 110.915 | 1.00 | 98.49 |
| 306 | OD1 | ASP | A | 262 | −0.729 | 64.514 | 110.865 | 1.00 | 98.82 |
| 307 | OD2 | ASP | A | 262 | −1.884 | 63.091 | 112.109 | 1.00 | 96.43 |
| 308 | C | ASP | A | 262 | 0.289 | 62.562 | 108.232 | 1.00 | 83.28 |
| 309 | O | ASP | A | 262 | 1.166 | 63.340 | 108.472 | 1.00 | 79.22 |
| 310 | N | ALA | A | 263 | 0.501 | 61.324 | 107.911 | 1.00 | 79.80 |
| 311 | CA | ALA | A | 263 | 1.734 | 60.758 | 107.923 | 1.00 | 80.97 |
| 312 | CB | ALA | A | 263 | 1.643 | 59.237 | 107.933 | 1.00 | 80.93 |
| 313 | C | ALA | A | 263 | 2.631 | 61.323 | 106.771 | 1.00 | 82.33 |
| 314 | O | ALA | A | 263 | 3.839 | 61.747 | 107.036 | 1.00 | 78.28 |
| 315 | N | ARG | A | 264 | 1.990 | 61.404 | 105.605 | 1.00 | 81.80 |
| 316 | CA | ARG | A | 264 | 2.549 | 62.024 | 104.495 | 1.00 | 80.25 |
| 317 | CB | ARG | A | 264 | 1.761 | 61.997 | 103.248 | 1.00 | 78.27 |
| 318 | CG | ARG | A | 264 | 2.546 | 62.624 | 102.058 | 1.00 | 86.83 |
| 319 | CD | ARG | A | 264 | 2.165 | 62.205 | 100.699 | 1.00 | 85.55 |
| 320 | NE | ARG | A | 264 | 0.897 | 62.764 | 100.331 | 1.00 | 98.12 |
| 321 | CZ | ARG | A | 264 | 0.405 | 62.740 | 99.061 | 1.00 | 105.97 |
| 322 | NH1 | ARG | A | 264 | 1.140 | 62.255 | 98.026 | 1.00 | 97.67 |
| 323 | NH2 | ARG | A | 264 | −0.870 | 63.206 | 98.822 | 1.00 | 112.57 |
| 324 | C | ARG | A | 264 | 3.147 | 63.361 | 104.847 | 1.00 | 81.58 |
| 325 | O | ARG | A | 264 | 4.483 | 63.647 | 104.638 | 1.00 | 83.41 |
| 326 | N | GLN | A | 265 | 2.342 | 64.122 | 105.517 | 1.00 | 79.93 |
| 327 | CA | GLN | A | 265 | 2.863 | 65.499 | 105.847 | 1.00 | 81.17 |
| 328 | CB | GLN | A | 265 | 1.635 | 66.454 | 106.366 | 1.00 | 76.97 |
| 329 | CG | GLN | A | 265 | 2.165 | 68.042 | 106.549 | 1.00 | 74.71 |
| 330 | CD | GLN | A | 265 | 2.449 | 68.677 | 105.089 | 1.00 | 84.93 |
| 331 | OE1 | GLN | A | 265 | 2.513 | 67.904 | 104.022 | 1.00 | 92.74 |
| 332 | NE2 | GLN | A | 265 | 2.537 | 69.964 | 104.979 | 1.00 | 82.66 |
| 333 | C | GLN | A | 265 | 4.059 | 65.441 | 106.811 | 1.00 | 73.92 |
| 334 | O | GLN | A | 265 | 4.932 | 66.164 | 106.657 | 1.00 | 70.94 |
| 335 | N | GLN | A | 266 | 3.929 | 64.519 | 107.799 | 1.00 | 73.59 |
| 336 | CA | GLN | A | 266 | 4.927 | 64.390 | 108.780 | 1.00 | 75.99 |
| 337 | CB | GLN | A | 266 | 4.713 | 63.205 | 109.660 | 1.00 | 78.43 |
| 338 | CG | GLN | A | 266 | 3.870 | 63.514 | 110.967 | 1.00 | 90.20 |
| 339 | CD | GLN | A | 266 | 3.961 | 62.361 | 111.974 | 1.00 | 95.67 |
| 340 | OE1 | GLN | A | 266 | 5.119 | 62.116 | 112.694 | 1.00 | 93.72 |
| 341 | NE2 | GLN | A | 266 | 2.764 | 61.670 | 112.138 | 1.00 | 85.07 |
| 342 | C | GLN | A | 266 | 6.389 | 64.303 | 108.113 | 1.00 | 72.79 |
| 343 | O | GLN | A | 266 | 7.354 | 64.959 | 108.523 | 1.00 | 68.40 |
| 344 | N | ARG | A | 267 | 6.500 | 63.418 | 107.183 | 1.00 | 71.03 |
| 345 | CA | ARG | A | 267 | 7.795 | 63.115 | 106.615 | 1.00 | 69.36 |
| 346 | CB | ARG | A | 267 | 7.878 | 61.736 | 106.087 | 1.00 | 68.79 |
| 347 | CG | ARG | A | 267 | 6.764 | 61.073 | 105.456 | 1.00 | 70.16 |
| 348 | CD | ARG | A | 267 | 6.749 | 59.637 | 105.649 | 1.00 | 68.84 |
| 349 | NE | ARG | A | 267 | 5.458 | 59.074 | 105.072 | 1.00 | 95.99 |
| 350 | CZ | ARG | A | 267 | 4.611 | 58.178 | 105.644 | 1.00 | 81.18 |
| 351 | NH1 | ARG | A | 267 | 4.908 | 57.599 | 106.858 | 1.00 | 69.37 |
| 352 | NH2 | ARG | A | 267 | 3.576 | 57.980 | 104.906 | 1.00 | 79.08 |
| 353 | C | ARG | A | 267 | 8.188 | 64.191 | 105.527 | 1.00 | 67.58 |
| 354 | O | ARG | A | 267 | 9.252 | 64.618 | 105.548 | 1.00 | 62.90 |
| 355 | N | PHE | A | 268 | 7.281 | 64.706 | 104.844 | 1.00 | 60.14 |
| 356 | CA | PHE | A | 268 | 7.581 | 65.759 | 104.173 | 1.00 | 57.88 |
| 357 | CB | PHE | A | 268 | 6.506 | 66.256 | 103.170 | 1.00 | 58.70 |
| 358 | CG | PHE | A | 268 | 6.780 | 67.668 | 102.543 | 1.00 | 58.81 |
| 359 | CD1 | PHE | A | 268 | 6.475 | 68.803 | 103.236 | 1.00 | 55.39 |
| 360 | CE1 | PHE | A | 268 | 6.467 | 70.043 | 102.736 | 1.00 | 53.30 |
| 361 | CZ | PHE | A | 268 | 7.111 | 70.158 | 101.441 | 1.00 | 62.89 |
| 362 | CE2 | PHE | A | 268 | 7.479 | 69.081 | 100.800 | 1.00 | 62.35 |
| 363 | CD2 | PHE | A | 268 | 7.290 | 67.757 | 101.374 | 1.00 | 58.36 |
| 364 | C | PHE | A | 268 | 8.190 | 66.807 | 104.855 | 1.00 | 57.70 |
| 365 | O | PHE | A | 268 | 9.280 | 67.425 | 104.500 | 1.00 | 58.64 |
| 366 | N | ALA | A | 269 | 7.596 | 67.091 | 105.887 | 1.00 | 56.07 |
| 367 | CA | ALA | A | 269 | 8.134 | 68.161 | 106.730 | 1.00 | 51.01 |
| 368 | CB | ALA | A | 269 | 6.867 | 68.463 | 107.890 | 1.00 | 55.80 |
| 369 | C | ALA | A | 269 | 9.366 | 67.877 | 107.323 | 1.00 | 44.97 |
| 370 | O | ALA | A | 269 | 10.056 | 68.732 | 107.752 | 1.00 | 46.96 |
| 371 | N | HIS | A | 270 | 9.506 | 66.632 | 107.587 | 1.00 | 45.75 |
| 372 | CA | HIS | A | 270 | 10.624 | 65.965 | 108.205 | 1.00 | 45.27 |
| 373 | CB | HIS | A | 270 | 10.428 | 64.358 | 108.271 | 1.00 | 39.46 |
| 374 | CG | HIS | A | 270 | 11.596 | 63.579 | 108.555 | 1.00 | 41.35 |
| 375 | ND1 | HIS | A | 270 | 12.016 | 63.285 | 109.849 | 1.00 | 52.22 |
| 376 | CE1 | HIS | A | 270 | 13.145 | 62.576 | 109.839 | 1.00 | 44.10 |
| 377 | NE2 | HIS | A | 270 | 13.506 | 62.476 | 108.575 | 1.00 | 45.77 |
| 378 | CD2 | HIS | A | 270 | 12.575 | 63.037 | 107.702 | 1.00 | 40.47 |
| 379 | C | HIS | A | 270 | 11.888 | 66.365 | 107.194 | 1.00 | 43.21 |
| 380 | O | HIS | A | 270 | 12.903 | 66.708 | 107.731 | 1.00 | 42.38 |
| 381 | N | PHE | A | 271 | 11.673 | 66.108 | 105.982 | 1.00 | 39.83 |
| 382 | CA | PHE | A | 271 | 12.588 | 66.267 | 104.971 | 1.00 | 49.60 |
| 383 | CB | PHE | A | 271 | 12.108 | 65.888 | 103.577 | 1.00 | 41.02 |
| 384 | CG | PHE | A | 271 | 12.208 | 64.333 | 103.457 | 1.00 | 45.71 |
| 385 | CD1 | PHE | A | 271 | 13.357 | 63.618 | 103.931 | 1.00 | 46.76 |
| 386 | CE1 | PHE | A | 271 | 13.520 | 62.289 | 103.766 | 1.00 | 45.86 |
| 387 | CZ | PHE | A | 271 | 12.552 | 61.512 | 103.235 | 1.00 | 52.48 |
| 388 | CE2 | PHE | A | 271 | 11.341 | 62.249 | 102.795 | 1.00 | 53.70 |
| 389 | CD2 | PHE | A | 271 | 11.333 | 63.686 | 102.941 | 1.00 | 51.87 |
| 390 | C | PHE | A | 271 | 13.106 | 67.901 | 104.921 | 1.00 | 55.37 |
| 391 | O | PHE | A | 271 | 14.338 | 68.174 | 104.794 | 1.00 | 43.89 |
| 392 | N | THR | A | 272 | 12.028 | 68.835 | 104.905 | 1.00 | 48.02 |
| 393 | CA | THR | A | 272 | 12.280 | 70.108 | 104.829 | 1.00 | 46.08 |
| 394 | CB | THR | A | 272 | 11.040 | 70.934 | 104.818 | 1.00 | 49.72 |
| 395 | OG1 | THR | A | 272 | 10.377 | 70.754 | 106.059 | 1.00 | 61.56 |
| 396 | CG2 | THR | A | 272 | 10.155 | 70.798 | 103.621 | 1.00 | 46.29 |
| 397 | C | THR | A | 272 | 13.104 | 70.535 | 106.046 | 1.00 | 48.09 |
| 398 | O | THR | A | 272 | 14.062 | 71.485 | 105.902 | 1.00 | 46.74 |
| 399 | N | GLU | A | 273 | 12.921 | 69.900 | 107.131 | 1.00 | 43.20 |
| 400 | CA | GLU | A | 273 | 13.665 | 70.298 | 108.300 | 1.00 | 45.93 |
| 401 | CB | GLU | A | 273 | 12.995 | 69.744 | 109.651 | 1.00 | 41.12 |
| 402 | CG | GLU | A | 273 | 11.618 | 70.435 | 109.957 | 1.00 | 51.85 |
| 403 | CD | GLU | A | 273 | 10.548 | 69.697 | 110.822 | 1.00 | 57.32 |
| 404 | OE1 | GLU | A | 273 | 9.213 | 69.838 | 110.486 | 1.00 | 59.67 |
| 405 | OE2 | GLU | A | 273 | 10.992 | 68.926 | 111.572 | 1.00 | 54.48 |
| 406 | C | GLU | A | 273 | 15.116 | 69.827 | 108.243 | 1.00 | 45.61 |
| 407 | O | GLU | A | 273 | 16.031 | 70.436 | 108.840 | 1.00 | 44.13 |
| 408 | N | LEU | A | 274 | 15.279 | 68.724 | 107.592 | 1.00 | 43.53 |
| 409 | CA | LEU | A | 274 | 16.710 | 68.181 | 107.376 | 1.00 | 46.73 |
| 410 | CB | LEU | A | 274 | 16.702 | 66.703 | 106.832 | 1.00 | 44.16 |
| 411 | CG | LEU | A | 274 | 16.085 | 65.666 | 107.751 | 1.00 | 47.12 |
| 412 | CD1 | LEU | A | 274 | 16.213 | 64.321 | 107.207 | 1.00 | 49.05 |
| 413 | CD2 | LEU | A | 274 | 16.793 | 65.661 | 109.189 | 1.00 | 53.81 |
| 414 | C | LEU | A | 274 | 17.380 | 69.082 | 106.296 | 1.00 | 37.65 |
| 415 | O | LEU | A | 274 | 18.559 | 69.429 | 106.475 | 1.00 | 40.99 |
| 416 | N | ALA | A | 275 | 16.651 | 69.567 | 105.389 | 1.00 | 36.60 |
| 417 | CA | ALA | A | 275 | 17.202 | 70.457 | 104.430 | 1.00 | 38.52 |
| 418 | CB | ALA | A | 275 | 16.224 | 70.659 | 103.268 | 1.00 | 39.37 |
| 419 | C | ALA | A | 275 | 17.691 | 71.778 | 105.001 | 1.00 | 40.85 |
| 420 | O | ALA | A | 275 | 18.752 | 72.357 | 104.671 | 1.00 | 38.61 |
| 421 | N | ILE | A | 276 | 16.877 | 72.342 | 105.919 | 1.00 | 42.17 |
| 422 | CA | ILE | A | 276 | 17.186 | 73.531 | 106.488 | 1.00 | 40.42 |
| 423 | CB | ILE | A | 276 | 15.979 | 73.854 | 107.589 | 1.00 | 44.47 |
| 424 | CG1 | ILE | A | 276 | 14.852 | 74.498 | 106.855 | 1.00 | 39.46 |
| 425 | CD1 | ILE | A | 276 | 13.541 | 74.531 | 107.865 | 1.00 | 41.41 |
| 426 | CG2 | ILE | A | 276 | 16.526 | 74.936 | 108.670 | 1.00 | 35.38 |
| 427 | C | ILE | A | 276 | 18.506 | 73.478 | 107.236 | 1.00 | 37.69 |
| 428 | O | ILE | A | 276 | 19.269 | 74.309 | 107.080 | 1.00 | 38.14 |
| 429 | N | ILE | A | 277 | 18.699 | 72.477 | 108.088 | 1.00 | 38.21 |
| 430 | CA | ILE | A | 277 | 19.862 | 72.336 | 108.757 | 1.00 | 39.69 |
| 431 | CB | ILE | A | 277 | 19.902 | 71.185 | 109.703 | 1.00 | 39.90 |
| 432 | CG1 | ILE | A | 277 | 20.708 | 70.218 | 109.290 | 1.00 | 48.55 |
| 433 | CD1 | ILE | A | 277 | 22.031 | 70.004 | 110.001 | 1.00 | 41.91 |
| 434 | CG2 | ILE | A | 277 | 18.381 | 70.692 | 110.105 | 1.00 | 40.45 |
| 435 | C | ILE | A | 277 | 21.048 | 72.223 | 107.854 | 1.00 | 42.32 |
| 436 | O | ILE | A | 277 | 22.135 | 72.732 | 108.134 | 1.00 | 43.22 |
| 437 | N | SER | A | 278 | 20.826 | 71.625 | 106.662 | 1.00 | 40.01 |
| 438 | CA | SER | A | 278 | 21.900 | 71.440 | 105.603 | 1.00 | 40.22 |
| 439 | CB | SER | A | 278 | 21.478 | 70.430 | 104.608 | 1.00 | 42.15 |
| 440 | OG | SER | A | 278 | 22.347 | 70.093 | 103.788 | 1.00 | 45.60 |
| 441 | C | SER | A | 278 | 22.213 | 72.786 | 104.954 | 1.00 | 39.58 |
| 442 | O | SER | A | 278 | 23.334 | 73.158 | 104.791 | 1.00 | 36.14 |
| 443 | N | VAL | A | 279 | 21.143 | 73.566 | 104.698 | 1.00 | 39.92 |
| 444 | CA | VAL | A | 279 | 21.339 | 74.810 | 104.187 | 1.00 | 38.47 |
| 445 | CB | VAL | A | 279 | 20.056 | 75.584 | 104.049 | 1.00 | 39.82 |
| 446 | CG1 | VAL | A | 279 | 20.358 | 77.235 | 103.798 | 1.00 | 35.21 |
| 447 | CG2 | VAL | A | 279 | 19.247 | 75.129 | 102.844 | 1.00 | 34.92 |
| 448 | C | VAL | A | 279 | 22.329 | 75.561 | 105.095 | 1.00 | 45.22 |
| 449 | O | VAL | A | 279 | 23.125 | 76.471 | 104.647 | 1.00 | 42.04 |
| 450 | N | GLN | A | 280 | 22.150 | 75.400 | 106.392 | 1.00 | 45.82 |
| 451 | CA | GLN | A | 280 | 22.964 | 76.151 | 107.344 | 1.00 | 46.04 |
| 452 | CB | GLN | A | 280 | 22.405 | 76.092 | 108.992 | 1.00 | 55.38 |
| 453 | CG | GLN | A | 280 | 21.139 | 76.574 | 109.143 | 1.00 | 62.18 |
| 454 | CD | GLN | A | 280 | 20.329 | 75.819 | 110.251 | 1.00 | 68.24 |
| 455 | OE1 | GLN | A | 280 | 19.182 | 75.919 | 110.230 | 1.00 | 72.94 |
| 456 | NE2 | GLN | A | 280 | 21.036 | 74.983 | 111.202 | 1.00 | 81.56 |
| 457 | C | GLN | A | 280 | 24.316 | 75.708 | 107.475 | 1.00 | 38.58 |
| 458 | O | GLN | A | 280 | 25.206 | 76.572 | 107.578 | 1.00 | 42.13 |
| 459 | N | GLU | A | 281 | 24.507 | 74.458 | 107.521 | 1.00 | 35.85 |
| 460 | CA | GLU | A | 281 | 25.926 | 73.894 | 107.421 | 1.00 | 35.48 |
| 461 | CB | GLU | A | 281 | 25.783 | 72.340 | 107.247 | 1.00 | 49.21 |
| 462 | CG | GLU | A | 281 | 27.013 | 71.521 | 107.283 | 1.00 | 46.94 |
| 463 | CD | GLU | A | 281 | 26.679 | 70.045 | 107.345 | 1.00 | 55.11 |
| 464 | OE1 | GLU | A | 281 | 27.566 | 69.080 | 107.089 | 1.00 | 62.34 |
| 465 | OE2 | GLU | A | 281 | 25.561 | 69.778 | 107.501 | 1.00 | 54.34 |
| 466 | C | GLU | A | 281 | 26.557 | 74.462 | 106.211 | 1.00 | 36.88 |
| 467 | O | GLU | A | 281 | 27.705 | 75.002 | 106.324 | 1.00 | 40.12 |
| 468 | N | ILE | A | 282 | 25.873 | 74.468 | 105.032 | 1.00 | 32.93 |
| 469 | CA | ILE | A | 282 | 26.483 | 74.937 | 103.824 | 1.00 | 37.10 |
| 470 | CB | ILE | A | 282 | 25.563 | 74.673 | 102.623 | 1.00 | 36.60 |
| 471 | CG1 | ILE | A | 282 | 25.542 | 73.172 | 102.424 | 1.00 | 33.12 |
| 472 | CD1 | ILE | A | 282 | 24.398 | 72.648 | 101.607 | 1.00 | 28.41 |
| 473 | CG2 | ILE | A | 282 | 26.058 | 75.501 | 101.462 | 1.00 | 34.63 |
| 474 | C | ILE | A | 282 | 26.812 | 76.328 | 103.903 | 1.00 | 36.22 |
| 475 | O | ILE | A | 282 | 27.931 | 76.779 | 103.498 | 1.00 | 35.64 |
| 476 | N | VAL | A | 283 | 25.928 | 77.183 | 104.449 | 1.00 | 35.19 |
| 477 | CA | VAL | A | 283 | 26.314 | 78.657 | 104.550 | 1.00 | 35.61 |
| 478 | CB | VAL | A | 283 | 25.197 | 79.416 | 105.251 | 1.00 | 49.06 |
| 479 | CG1 | VAL | A | 283 | 25.552 | 80.798 | 105.622 | 1.00 | 41.24 |
| 480 | CG2 | VAL | A | 283 | 23.909 | 79.435 | 104.238 | 1.00 | 38.80 |
| 481 | C | VAL | A | 283 | 27.442 | 78.860 | 105.564 | 1.00 | 41.26 |
| 482 | O | VAL | A | 283 | 28.388 | 79.567 | 105.271 | 1.00 | 41.99 |
| 483 | N | ASP | A | 284 | 27.591 | 77.996 | 106.639 | 1.00 | 38.72 |
| 484 | CA | ASP | A | 284 | 28.711 | 78.049 | 107.437 | 1.00 | 38.65 |
| 485 | CB | ASP | A | 284 | 28.573 | 77.059 | 108.724 | 1.00 | 46.77 |
| 486 | CG | ASP | A | 284 | 27.750 | 77.687 | 109.857 | 1.00 | 55.54 |
| 487 | OD1 | ASP | A | 284 | 27.540 | 78.969 | 109.916 | 1.00 | 60.09 |
| 488 | OD2 | ASP | A | 284 | 27.147 | 76.899 | 110.567 | 1.00 | 63.52 |
| 489 | C | ASP | A | 284 | 29.957 | 77.605 | 106.801 | 1.00 | 39.66 |
| 490 | O | ASP | A | 284 | 31.004 | 78.247 | 107.049 | 1.00 | 39.55 |
| 491 | N | PHE | A | 285 | 29.933 | 76.487 | 106.050 | 1.00 | 34.06 |
| 492 | CA | PHE | A | 285 | 31.170 | 76.046 | 105.376 | 1.00 | 32.16 |
| 493 | CB | PHE | A | 285 | 30.800 | 74.806 | 104.501 | 1.00 | 32.46 |
| 494 | CG | PHE | A | 285 | 31.957 | 74.319 | 103.589 | 1.00 | 35.38 |
| 495 | CD1 | PHE | A | 285 | 33.006 | 73.657 | 104.233 | 1.00 | 35.99 |
| 496 | CE1 | PHE | A | 285 | 34.102 | 73.254 | 103.450 | 1.00 | 31.35 |
| 497 | CZ | PHE | A | 285 | 34.040 | 73.480 | 101.977 | 1.00 | 37.66 |
| 498 | CE2 | PHE | A | 285 | 32.990 | 74.139 | 101.494 | 1.00 | 33.00 |
| 499 | CD2 | PHE | A | 285 | 32.024 | 74.562 | 102.287 | 1.00 | 29.15 |
| 500 | C | PHE | A | 285 | 31.637 | 77.114 | 104.372 | 1.00 | 34.62 |
| 501 | O | PHE | A | 285 | 32.781 | 77.476 | 104.358 | 1.00 | 37.72 |
| 502 | N | ALA | A | 286 | 30.745 | 77.696 | 103.563 | 1.00 | 36.82 |
| 503 | CA | ALA | A | 286 | 31.084 | 78.702 | 102.552 | 1.00 | 36.74 |
| 504 | CB | ALA | A | 286 | 29.775 | 79.238 | 101.842 | 1.00 | 29.00 |
| 505 | C | ALA | A | 286 | 31.849 | 79.881 | 103.085 | 1.00 | 36.02 |
| 506 | O | ALA | A | 286 | 32.839 | 80.338 | 102.523 | 1.00 | 33.70 |
| 507 | N | LYS | A | 287 | 31.424 | 80.349 | 104.251 | 1.00 | 39.16 |
| 508 | CA | LYS | A | 287 | 32.048 | 81.465 | 104.851 | 1.00 | 37.96 |
| 509 | CB | LYS | A | 287 | 31.225 | 81.937 | 106.082 | 1.00 | 41.83 |
| 510 | CG | LYS | A | 287 | 29.862 | 82.657 | 105.722 | 1.00 | 45.12 |
| 511 | CD | LYS | A | 287 | 29.353 | 83.213 | 107.138 | 1.00 | 49.61 |
| 512 | CE | LYS | A | 287 | 28.172 | 83.922 | 106.877 | 1.00 | 61.11 |
| 513 | NZ | LYS | A | 287 | 27.335 | 84.148 | 108.309 | 1.00 | 63.99 |
| 514 | C | LYS | A | 287 | 33.423 | 81.108 | 105.370 | 1.00 | 42.47 |
| 515 | O | LYS | A | 287 | 34.187 | 81.974 | 105.686 | 1.00 | 43.87 |
| 516 | N | GLN | A | 288 | 33.785 | 79.855 | 105.415 | 1.00 | 37.42 |
| 517 | CA | GLN | A | 288 | 35.135 | 79.594 | 105.830 | 1.00 | 38.23 |
| 518 | CB | GLN | A | 288 | 35.120 | 78.300 | 106.750 | 1.00 | 44.53 |
| 519 | CG | GLN | A | 288 | 35.041 | 78.737 | 108.331 | 1.00 | 49.18 |
| 520 | CD | GLN | A | 288 | 34.698 | 77.600 | 109.135 | 1.00 | 64.09 |
| 521 | OE1 | GLN | A | 288 | 33.359 | 77.135 | 109.051 | 1.00 | 67.22 |
| 522 | NE2 | GLN | A | 288 | 35.667 | 76.969 | 109.849 | 1.00 | 52.35 |
| 523 | C | GLN | A | 288 | 36.014 | 79.205 | 104.588 | 1.00 | 41.53 |
| 524 | O | GLN | A | 288 | 37.165 | 78.881 | 104.710 | 1.00 | 37.36 |
| 525 | N | VAL | A | 289 | 35.399 | 79.288 | 103.371 | 1.00 | 37.03 |
| 526 | CA | VAL | A | 289 | 36.199 | 79.035 | 102.188 | 1.00 | 35.20 |
| 527 | CB | VAL | A | 289 | 35.351 | 78.477 | 101.018 | 1.00 | 34.98 |
| 528 | CG1 | VAL | A | 289 | 36.061 | 78.437 | 99.707 | 1.00 | 27.92 |
| 529 | CG2 | VAL | A | 289 | 34.811 | 77.046 | 101.400 | 1.00 | 23.48 |
| 530 | C | VAL | A | 289 | 36.982 | 80.255 | 101.840 | 1.00 | 34.92 |
| 531 | O | VAL | A | 289 | 36.453 | 81.265 | 101.702 | 1.00 | 36.02 |
| 532 | N | PRO | A | 290 | 38.322 | 80.172 | 101.764 | 1.00 | 37.23 |
| 533 | CA | PRO | A | 290 | 39.182 | 81.368 | 101.488 | 1.00 | 37.79 |
| 534 | CB | PRO | A | 290 | 40.557 | 80.827 | 101.405 | 1.00 | 33.46 |
| 535 | CG | PRO | A | 290 | 40.570 | 79.722 | 102.556 | 1.00 | 33.40 |
| 536 | CD | PRO | A | 290 | 39.192 | 78.974 | 102.164 | 1.00 | 34.43 |
| 537 | C | PRO | A | 290 | 38.794 | 82.090 | 100.251 | 1.00 | 38.43 |
| 538 | O | PRO | A | 290 | 38.684 | 81.423 | 99.169 | 1.00 | 41.81 |
| 539 | N | GLY | A | 291 | 38.546 | 83.337 | 100.306 | 1.00 | 35.20 |
| 540 | CA | GLY | A | 291 | 38.177 | 84.204 | 99.153 | 1.00 | 32.43 |
| 541 | C | GLY | A | 291 | 36.583 | 84.456 | 99.134 | 1.00 | 34.08 |
| 542 | O | GLY | A | 291 | 36.164 | 85.491 | 98.633 | 1.00 | 34.21 |
| 543 | N | PHE | A | 292 | 35.783 | 83.615 | 99.677 | 1.00 | 30.18 |
| 544 | CA | PHE | A | 292 | 34.369 | 83.906 | 99.628 | 1.00 | 32.64 |
| 545 | CB | PHE | A | 292 | 33.584 | 82.739 | 100.290 | 1.00 | 29.03 |
| 546 | CG | PHE | A | 292 | 32.075 | 82.790 | 100.082 | 1.00 | 30.95 |
| 547 | CD1 | PHE | A | 292 | 31.467 | 82.417 | 98.840 | 1.00 | 29.16 |
| 548 | CE1 | PHE | A | 292 | 30.172 | 82.389 | 98.678 | 1.00 | 29.51 |
| 549 | CZ | PHE | A | 292 | 29.187 | 82.781 | 99.831 | 1.00 | 29.82 |
| 550 | CE2 | PHE | A | 292 | 29.833 | 83.315 | 100.964 | 1.00 | 23.75 |
| 551 | CD2 | PHE | A | 292 | 31.206 | 83.313 | 101.160 | 1.00 | 26.41 |
| 552 | C | PHE | A | 292 | 33.957 | 85.205 | 100.213 | 1.00 | 36.18 |
| 553 | O | PHE | A | 292 | 32.977 | 85.823 | 99.748 | 1.00 | 37.86 |
| 554 | N | LEU | A | 293 | 34.587 | 85.654 | 101.303 | 1.00 | 41.21 |
| 555 | CA | LEU | A | 293 | 34.198 | 86.932 | 102.011 | 1.00 | 41.20 |
| 556 | CB | LEU | A | 293 | 34.586 | 86.881 | 103.397 | 1.00 | 39.17 |
| 557 | CG | LEU | A | 293 | 33.915 | 85.823 | 104.283 | 1.00 | 39.38 |
| 558 | CD1 | LEU | A | 293 | 34.284 | 85.790 | 105.797 | 1.00 | 38.40 |
| 559 | CD2 | LEU | A | 293 | 32.322 | 86.131 | 104.340 | 1.00 | 40.30 |
| 560 | C | LEU | A | 293 | 34.802 | 88.115 | 101.349 | 1.00 | 42.23 |
| 561 | O | LEU | A | 293 | 34.300 | 89.237 | 101.556 | 1.00 | 41.57 |
| 562 | N | GLN | A | 294 | 35.665 | 87.896 | 100.338 | 1.00 | 38.50 |
| 563 | CA | GLN | A | 294 | 36.202 | 89.108 | 99.660 | 1.00 | 38.07 |
| 564 | CB | GLN | A | 294 | 37.558 | 88.679 | 98.896 | 1.00 | 43.74 |
| 565 | CG | GLN | A | 294 | 38.741 | 88.503 | 99.899 | 1.00 | 45.87 |
| 566 | CD | GLN | A | 294 | 39.847 | 87.812 | 99.200 | 1.00 | 57.56 |
| 567 | OE1 | GLN | A | 294 | 40.862 | 87.334 | 99.953 | 1.00 | 71.17 |
| 568 | NE2 | GLN | A | 294 | 39.921 | 87.843 | 97.758 | 1.00 | 55.46 |
| 569 | C | GLN | A | 294 | 35.258 | 89.437 | 98.561 | 1.00 | 38.66 |
| 570 | O | GLN | A | 294 | 35.466 | 90.400 | 97.831 | 1.00 | 38.79 |
| 571 | N | LEU | A | 295 | 34.155 | 88.691 | 98.405 | 1.00 | 36.89 |
| 572 | CA | LEU | A | 295 | 33.103 | 89.116 | 97.416 | 1.00 | 31.31 |
| 573 | CB | LEU | A | 295 | 32.445 | 87.883 | 96.889 | 1.00 | 28.74 |
| 574 | CG | LEU | A | 295 | 33.323 | 87.020 | 95.927 | 1.00 | 29.80 |
| 575 | CD1 | LEU | A | 295 | 32.637 | 85.475 | 95.793 | 1.00 | 25.37 |
| 576 | CD2 | LEU | A | 295 | 33.453 | 87.750 | 94.464 | 1.00 | 22.18 |
| 577 | C | LEU | A | 295 | 32.165 | 89.985 | 98.121 | 1.00 | 30.65 |
| 578 | O | LEU | A | 295 | 31.913 | 89.837 | 99.395 | 1.00 | 30.96 |
| 579 | N | GLY | A | 296 | 31.419 | 90.721 | 97.364 | 1.00 | 33.64 |
| 580 | CA | GLY | A | 296 | 30.282 | 91.558 | 97.960 | 1.00 | 34.26 |
| 581 | C | GLY | A | 296 | 29.253 | 90.658 | 98.472 | 1.00 | 38.96 |
| 582 | O | GLY | A | 296 | 29.183 | 89.442 | 98.088 | 1.00 | 33.27 |
| 583 | N | ARG | A | 297 | 28.330 | 91.197 | 99.373 | 1.00 | 36.07 |
| 584 | CA | ARG | A | 297 | 27.400 | 90.404 | 99.968 | 1.00 | 31.87 |
| 585 | CB | ARG | A | 297 | 26.541 | 91.369 | 100.901 | 1.00 | 41.49 |
| 586 | CG | ARG | A | 297 | 25.152 | 90.646 | 101.517 | 1.00 | 42.67 |
| 587 | CD | ARG | A | 297 | 24.353 | 91.552 | 102.368 | 1.00 | 57.56 |
| 588 | NE | ARG | A | 297 | 22.938 | 91.051 | 102.888 | 1.00 | 57.97 |
| 589 | CZ | ARG | A | 297 | 22.911 | 89.948 | 103.622 | 1.00 | 60.51 |
| 590 | NH1 | ARG | A | 297 | 24.123 | 89.340 | 103.925 | 1.00 | 56.84 |
| 591 | NH2 | ARG | A | 297 | 21.759 | 89.300 | 103.920 | 1.00 | 51.82 |
| 592 | C | ARG | A | 297 | 26.495 | 89.884 | 98.843 | 1.00 | 31.57 |
| 593 | O | ARG | A | 297 | 25.980 | 88.825 | 98.896 | 1.00 | 32.83 |
| 594 | N | GLU | A | 298 | 26.102 | 90.797 | 97.920 | 1.00 | 34.95 |
| 595 | CA | GLU | A | 298 | 25.254 | 90.424 | 96.907 | 1.00 | 31.54 |
| 596 | CB | GLU | A | 298 | 25.098 | 91.508 | 95.814 | 1.00 | 36.90 |
| 597 | CG | GLU | A | 298 | 24.397 | 92.795 | 96.272 | 1.00 | 49.73 |
| 598 | CD | GLU | A | 298 | 25.378 | 93.783 | 97.000 | 1.00 | 47.92 |
| 599 | OE1 | GLU | A | 298 | 26.530 | 93.382 | 97.482 | 1.00 | 41.29 |
| 600 | OE2 | GLU | A | 298 | 24.864 | 94.893 | 97.235 | 1.00 | 51.31 |
| 601 | C | GLU | A | 298 | 25.800 | 89.071 | 96.190 | 1.00 | 27.71 |
| 602 | O | GLU | A | 298 | 25.030 | 88.123 | 96.026 | 1.00 | 24.56 |
| 603 | N | ASP | A | 299 | 26.978 | 89.108 | 95.749 | 1.00 | 27.21 |
| 604 | CA | ASP | A | 299 | 27.557 | 87.886 | 95.122 | 1.00 | 29.51 |
| 605 | CB | ASP | A | 299 | 28.912 | 88.174 | 94.507 | 1.00 | 27.66 |
| 606 | CG | ASP | A | 299 | 28.756 | 88.835 | 93.084 | 1.00 | 27.40 |
| 607 | OD1 | ASP | A | 299 | 27.657 | 89.010 | 92.621 | 1.00 | 25.56 |
| 608 | OD2 | ASP | A | 299 | 29.783 | 89.143 | 92.441 | 1.00 | 30.55 |
| 609 | C | ASP | A | 299 | 27.588 | 86.627 | 96.040 | 1.00 | 29.89 |
| 610 | O | ASP | A | 299 | 27.284 | 85.521 | 95.579 | 1.00 | 32.13 |
| 611 | N | GLN | A | 300 | 27.798 | 86.770 | 97.355 | 1.00 | 28.64 |
| 612 | CA | GLN | A | 300 | 27.835 | 85.720 | 98.203 | 1.00 | 32.43 |
| 613 | CB | GLN | A | 300 | 28.239 | 86.128 | 99.711 | 1.00 | 28.61 |
| 614 | CG | GLN | A | 300 | 29.593 | 86.674 | 99.827 | 1.00 | 32.36 |
| 615 | CD | GLN | A | 300 | 29.871 | 87.335 | 101.248 | 1.00 | 32.93 |
| 616 | OE1 | GLN | A | 300 | 30.740 | 88.124 | 101.345 | 1.00 | 35.53 |
| 617 | NE2 | GLN | A | 300 | 29.140 | 87.059 | 102.102 | 1.00 | 29.24 |
| 618 | C | GLN | A | 300 | 26.507 | 85.010 | 98.138 | 1.00 | 30.55 |
| 619 | O | GLN | A | 300 | 26.410 | 83.784 | 98.174 | 1.00 | 27.91 |
| 620 | N | ILE | A | 301 | 25.542 | 85.840 | 98.143 | 1.00 | 32.70 |
| 621 | CA | ILE | A | 301 | 24.073 | 85.277 | 98.165 | 1.00 | 31.27 |
| 622 | CB | ILE | A | 301 | 23.059 | 86.481 | 98.415 | 1.00 | 33.15 |
| 623 | CG1 | ILE | A | 301 | 23.331 | 86.954 | 99.914 | 1.00 | 35.57 |
| 624 | CD1 | ILE | A | 301 | 22.305 | 88.193 | 100.298 | 1.00 | 30.35 |
| 625 | CG2 | ILE | A | 301 | 21.623 | 86.044 | 98.089 | 1.00 | 28.70 |
| 626 | C | ILE | A | 301 | 23.748 | 84.676 | 96.805 | 1.00 | 28.75 |
| 627 | O | ILE | A | 301 | 23.161 | 83.620 | 96.848 | 1.00 | 30.67 |
| 628 | N | ALA | A | 302 | 24.133 | 85.241 | 95.747 | 1.00 | 23.83 |
| 629 | CA | ALA | A | 302 | 23.746 | 84.638 | 94.490 | 1.00 | 30.56 |
| 630 | CB | ALA | A | 302 | 24.220 | 85.673 | 93.221 | 1.00 | 25.06 |
| 631 | C | ALA | A | 302 | 24.536 | 83.249 | 94.217 | 1.00 | 28.16 |
| 632 | O | ALA | A | 302 | 23.947 | 82.343 | 93.852 | 1.00 | 27.71 |
| 633 | N | LEU | A | 303 | 25.829 | 83.164 | 94.593 | 1.00 | 29.86 |
| 634 | CA | LEU | A | 303 | 26.527 | 81.954 | 94.511 | 1.00 | 31.92 |
| 635 | CB | LEU | A | 303 | 28.070 | 82.240 | 94.976 | 1.00 | 29.58 |
| 636 | CG | LEU | A | 303 | 28.750 | 83.153 | 93.983 | 1.00 | 31.40 |
| 637 | CD1 | LEU | A | 303 | 30.229 | 83.203 | 94.524 | 1.00 | 31.82 |
| 638 | CD2 | LEU | A | 303 | 28.685 | 82.579 | 92.531 | 1.00 | 24.33 |
| 639 | C | LEU | A | 303 | 26.019 | 80.903 | 95.445 | 1.00 | 30.24 |
| 640 | O | LEU | A | 303 | 25.844 | 79.752 | 95.063 | 1.00 | 33.92 |
| 641 | N | LEU | A | 304 | 25.633 | 81.290 | 96.655 | 1.00 | 31.43 |
| 642 | CA | LEU | A | 304 | 25.029 | 80.237 | 97.609 | 1.00 | 33.42 |
| 643 | CB | LEU | A | 304 | 24.984 | 80.782 | 98.962 | 1.00 | 29.58 |
| 644 | CG | LEU | A | 304 | 26.099 | 80.188 | 99.959 | 1.00 | 41.99 |
| 645 | CD1 | LEU | A | 304 | 25.744 | 78.928 | 100.571 | 1.00 | 43.21 |
| 646 | CD2 | LEU | A | 304 | 27.365 | 79.965 | 99.309 | 1.00 | 38.21 |
| 647 | C | LEU | A | 304 | 23.677 | 79.796 | 97.146 | 1.00 | 32.19 |
| 648 | O | LEU | A | 304 | 23.272 | 78.625 | 97.289 | 1.00 | 28.76 |
| 649 | N | LYS | A | 305 | 22.929 | 80.761 | 96.577 | 1.00 | 28.77 |
| 650 | CA | LYS | A | 305 | 21.523 | 80.408 | 96.180 | 1.00 | 29.45 |
| 651 | CB | LYS | A | 305 | 20.926 | 81.642 | 95.604 | 1.00 | 37.48 |
| 652 | CG | LYS | A | 305 | 19.491 | 81.504 | 95.045 | 1.00 | 42.80 |
| 653 | CD | LYS | A | 305 | 18.626 | 82.867 | 95.605 | 1.00 | 46.29 |
| 654 | CE | LYS | A | 305 | 17.714 | 83.286 | 94.535 | 1.00 | 47.99 |
| 655 | NZ | LYS | A | 305 | 16.658 | 82.418 | 94.316 | 1.00 | 47.49 |
| 656 | C | LYS | A | 305 | 21.578 | 79.366 | 94.968 | 1.00 | 29.76 |
| 657 | O | LYS | A | 305 | 20.765 | 78.372 | 94.990 | 1.00 | 29.63 |
| 658 | N | ALA | A | 306 | 22.456 | 79.535 | 94.094 | 1.00 | 24.49 |
| 659 | CA | ALA | A | 306 | 22.503 | 78.638 | 92.981 | 1.00 | 26.72 |
| 660 | CB | ALA | A | 306 | 23.216 | 79.225 | 91.740 | 1.00 | 17.94 |
| 661 | C | ALA | A | 306 | 23.301 | 77.341 | 93.404 | 1.00 | 27.93 |
| 662 | O | ALA | A | 306 | 22.931 | 76.218 | 92.857 | 1.00 | 28.47 |
| 663 | N | SER | A | 307 | 24.185 | 77.424 | 94.325 | 1.00 | 21.05 |
| 664 | CA | SER | A | 307 | 24.864 | 76.085 | 94.550 | 1.00 | 26.49 |
| 665 | CB | SER | A | 307 | 26.459 | 76.352 | 94.716 | 1.00 | 31.86 |
| 666 | OG | SER | A | 307 | 26.747 | 77.116 | 95.765 | 1.00 | 32.86 |
| 667 | C | SER | A | 307 | 24.442 | 75.252 | 95.719 | 1.00 | 26.76 |
| 668 | O | SER | A | 307 | 24.789 | 74.091 | 95.779 | 1.00 | 25.81 |
| 669 | N | THR | A | 308 | 23.483 | 75.743 | 96.543 | 1.00 | 26.28 |
| 670 | CA | THR | A | 308 | 23.022 | 75.056 | 97.654 | 1.00 | 27.51 |
| 671 | CB | THR | A | 308 | 21.941 | 75.893 | 98.398 | 1.00 | 27.16 |
| 672 | OG1 | THR | A | 308 | 22.578 | 76.918 | 99.064 | 1.00 | 29.21 |
| 673 | CG2 | THR | A | 308 | 21.261 | 74.912 | 99.449 | 1.00 | 17.76 |
| 674 | C | THR | A | 308 | 22.485 | 73.614 | 97.354 | 1.00 | 27.46 |
| 675 | O | THR | A | 308 | 22.802 | 72.648 | 98.066 | 1.00 | 24.75 |
| 676 | N | ILE | A | 309 | 21.545 | 73.538 | 96.378 | 1.00 | 26.93 |
| 677 | CA | ILE | A | 309 | 21.043 | 72.203 | 96.043 | 1.00 | 23.04 |
| 678 | CB | ILE | A | 309 | 19.812 | 72.359 | 94.990 | 1.00 | 26.72 |
| 679 | CG1 | ILE | A | 309 | 19.084 | 70.966 | 94.795 | 1.00 | 23.33 |
| 680 | CD1 | ILE | A | 309 | 18.514 | 70.435 | 96.105 | 1.00 | 23.17 |
| 681 | CG2 | ILE | A | 309 | 20.331 | 72.855 | 93.642 | 1.00 | 21.02 |
| 682 | C | ILE | A | 309 | 22.120 | 71.257 | 95.486 | 1.00 | 23.12 |
| 683 | O | ILE | A | 309 | 22.134 | 70.091 | 95.714 | 1.00 | 26.33 |
| 684 | N | GLU | A | 310 | 23.059 | 71.822 | 94.742 | 1.00 | 27.82 |
| 685 | CA | GLU | A | 310 | 24.208 | 71.093 | 94.160 | 1.00 | 22.59 |
| 686 | CB | GLU | A | 310 | 24.850 | 72.012 | 93.127 | 1.00 | 25.76 |
| 687 | CG | GLU | A | 310 | 23.984 | 72.351 | 91.953 | 1.00 | 26.34 |
| 688 | CD | GLU | A | 310 | 24.749 | 73.174 | 90.841 | 1.00 | 31.59 |
| 689 | OE1 | GLU | A | 310 | 25.997 | 73.129 | 90.798 | 1.00 | 21.67 |
| 690 | OE2 | GLU | A | 310 | 24.134 | 73.847 | 89.993 | 1.00 | 33.55 |
| 691 | C | GLU | A | 310 | 25.192 | 70.623 | 95.160 | 1.00 | 22.68 |
| 692 | O | GLU | A | 310 | 25.596 | 69.488 | 95.185 | 1.00 | 24.16 |
| 693 | N | ILE | A | 311 | 25.417 | 71.433 | 96.209 | 1.00 | 24.92 |
| 694 | CA | ILE | A | 311 | 26.276 | 70.946 | 97.306 | 1.00 | 24.59 |
| 695 | CB | ILE | A | 311 | 26.671 | 72.144 | 98.161 | 1.00 | 27.83 |
| 696 | CG1 | ILE | A | 311 | 27.596 | 73.082 | 97.376 | 1.00 | 27.28 |
| 697 | CD1 | ILE | A | 311 | 28.166 | 74.243 | 98.165 | 1.00 | 28.66 |
| 698 | CG2 | ILE | A | 311 | 27.445 | 71.689 | 99.477 | 1.00 | 24.31 |
| 699 | C | ILE | A | 311 | 25.603 | 69.910 | 98.096 | 1.00 | 26.73 |
| 700 | O | ILE | A | 311 | 26.163 | 68.877 | 98.518 | 1.00 | 25.76 |
| 701 | N | MET | A | 312 | 24.243 | 70.038 | 98.334 | 1.00 | 26.95 |
| 702 | CA | MET | A | 312 | 23.519 | 69.100 | 99.078 | 1.00 | 28.20 |
| 703 | CB | MET | A | 312 | 21.963 | 69.530 | 99.265 | 1.00 | 32.65 |
| 704 | CG | MET | A | 312 | 21.777 | 70.545 | 100.262 | 1.00 | 31.50 |
| 705 | SD | MET | A | 312 | 20.124 | 71.390 | 99.991 | 1.00 | 36.03 |
| 706 | CE | MET | A | 312 | 19.206 | 70.423 | 100.744 | 1.00 | 36.52 |
| 707 | C | MET | A | 312 | 23.544 | 67.684 | 98.337 | 1.00 | 25.43 |
| 708 | O | MET | A | 312 | 23.566 | 66.654 | 98.953 | 1.00 | 25.55 |
| 709 | N | LEU | A | 313 | 23.526 | 67.789 | 97.024 | 1.00 | 21.81 |
| 710 | CA | LEU | A | 313 | 23.605 | 66.542 | 96.263 | 1.00 | 21.12 |
| 711 | CB | LEU | A | 313 | 23.324 | 66.877 | 94.882 | 1.00 | 23.38 |
| 712 | CG | LEU | A | 313 | 21.760 | 67.105 | 94.603 | 1.00 | 25.11 |
| 713 | CD1 | LEU | A | 313 | 21.567 | 67.910 | 93.299 | 1.00 | 19.37 |
| 714 | CD2 | LEU | A | 313 | 21.209 | 65.754 | 94.499 | 1.00 | 27.49 |
| 715 | C | LEU | A | 313 | 25.053 | 65.882 | 96.420 | 1.00 | 20.35 |
| 716 | O | LEU | A | 313 | 25.151 | 64.736 | 96.646 | 1.00 | 19.92 |
| 717 | N | LEU | A | 314 | 26.069 | 66.745 | 96.347 | 1.00 | 24.80 |
| 718 | CA | LEU | A | 314 | 27.484 | 66.236 | 96.553 | 1.00 | 29.84 |
| 719 | CB | LEU | A | 314 | 28.457 | 67.428 | 96.397 | 1.00 | 24.30 |
| 720 | CG | LEU | A | 314 | 28.701 | 67.669 | 94.866 | 1.00 | 24.45 |
| 721 | CD1 | LEU | A | 314 | 29.562 | 69.031 | 94.743 | 1.00 | 29.60 |
| 722 | CD2 | LEU | A | 314 | 29.585 | 66.500 | 94.195 | 1.00 | 25.08 |
| 723 | C | LEU | A | 314 | 27.630 | 65.642 | 98.007 | 1.00 | 26.91 |
| 724 | O | LEU | A | 314 | 28.079 | 64.566 | 98.118 | 1.00 | 27.48 |
| 725 | N | GLU | A | 315 | 27.002 | 66.300 | 99.021 | 1.00 | 27.94 |
| 726 | CA | GLU | A | 315 | 27.090 | 65.741 | 100.410 | 1.00 | 27.46 |
| 727 | CB | GLU | A | 315 | 26.703 | 66.772 | 101.475 | 1.00 | 27.00 |
| 728 | CG | GLU | A | 315 | 27.660 | 67.838 | 101.618 | 1.00 | 35.38 |
| 729 | CD | GLU | A | 315 | 28.831 | 67.348 | 102.496 | 1.00 | 39.57 |
| 730 | OE1 | GLU | A | 315 | 28.643 | 66.362 | 103.285 | 1.00 | 41.14 |
| 731 | OE2 | GLU | A | 315 | 29.801 | 67.943 | 102.451 | 1.00 | 47.59 |
| 732 | C | GLU | A | 315 | 26.197 | 64.509 | 100.542 | 1.00 | 27.37 |
| 733 | O | GLU | A | 315 | 26.440 | 63.633 | 101.216 | 1.00 | 26.90 |
| 734 | N | THR | A | 316 | 25.111 | 64.467 | 99.789 | 1.00 | 29.35 |
| 735 | CA | THR | A | 316 | 24.282 | 63.248 | 99.855 | 1.00 | 25.85 |
| 736 | CB | THR | A | 316 | 22.955 | 63.569 | 98.999 | 1.00 | 25.11 |
| 737 | OG1 | THR | A | 316 | 22.209 | 64.367 | 99.778 | 1.00 | 24.63 |
| 738 | CG2 | THR | A | 316 | 22.277 | 62.300 | 98.644 | 1.00 | 30.27 |
| 739 | C | THR | A | 316 | 25.018 | 62.075 | 99.152 | 1.00 | 23.34 |
| 740 | O | THR | A | 316 | 25.182 | 61.015 | 99.749 | 1.00 | 25.72 |
| 741 | N | ALA | A | 317 | 25.713 | 62.346 | 97.969 | 1.00 | 21.51 |
| 742 | CA | ALA | A | 317 | 26.541 | 61.304 | 97.400 | 1.00 | 22.63 |
| 743 | CB | ALA | A | 317 | 27.287 | 61.849 | 96.075 | 1.00 | 21.57 |
| 744 | C | ALA | A | 317 | 27.720 | 60.896 | 98.383 | 1.00 | 26.32 |
| 745 | O | ALA | A | 317 | 27.894 | 59.724 | 98.569 | 1.00 | 26.21 |
| 746 | N | ARG | A | 318 | 28.285 | 61.849 | 99.070 | 1.00 | 24.26 |
| 747 | CA | ARG | A | 318 | 29.334 | 61.518 | 99.942 | 1.00 | 30.43 |
| 748 | CB | ARG | A | 318 | 30.036 | 62.850 | 100.434 | 1.00 | 28.78 |
| 749 | CG | ARG | A | 318 | 31.222 | 62.420 | 101.411 | 1.00 | 29.03 |
| 750 | CD | ARG | A | 318 | 31.994 | 63.794 | 101.741 | 1.00 | 35.38 |
| 751 | NE | ARG | A | 318 | 31.277 | 64.625 | 102.652 | 1.00 | 35.32 |
| 752 | CZ | ARG | A | 318 | 31.590 | 64.852 | 103.821 | 1.00 | 40.22 |
| 753 | NH1 | ARG | A | 318 | 32.773 | 64.250 | 104.443 | 1.00 | 42.05 |
| 754 | NH2 | ARG | A | 318 | 30.874 | 65.745 | 104.607 | 1.00 | 35.76 |
| 755 | C | ARG | A | 318 | 28.952 | 60.655 | 101.238 | 1.00 | 28.90 |
| 756 | O | ARG | A | 318 | 29.701 | 59.862 | 101.681 | 1.00 | 27.55 |
| 757 | N | ARG | A | 319 | 27.819 | 60.885 | 101.784 | 1.00 | 31.21 |
| 758 | CA | ARG | A | 319 | 27.401 | 60.142 | 102.999 | 1.00 | 27.42 |
| 759 | CB | ARG | A | 319 | 26.595 | 61.181 | 103.854 | 1.00 | 32.16 |
| 760 | CG | ARG | A | 319 | 27.289 | 62.317 | 104.256 | 1.00 | 37.82 |
| 761 | CD | ARG | A | 319 | 26.295 | 63.143 | 105.099 | 1.00 | 49.91 |
| 762 | NE | ARG | A | 319 | 26.949 | 64.323 | 105.979 | 1.00 | 59.32 |
| 763 | CZ | ARG | A | 319 | 27.151 | 65.577 | 105.576 | 1.00 | 60.21 |
| 764 | NH1 | ARG | A | 319 | 26.781 | 66.044 | 104.383 | 1.00 | 63.33 |
| 765 | NH2 | ARG | A | 319 | 27.618 | 66.510 | 106.498 | 1.00 | 67.19 |
| 766 | C | ARG | A | 319 | 26.450 | 58.968 | 102.592 | 1.00 | 28.30 |
| 767 | O | ARG | A | 319 | 25.897 | 58.308 | 103.478 | 1.00 | 31.01 |
| 768 | N | TYR | A | 320 | 26.148 | 58.776 | 101.300 | 1.00 | 26.84 |
| 769 | CA | TYR | A | 320 | 25.385 | 57.621 | 100.880 | 1.00 | 25.78 |
| 770 | CB | TYR | A | 320 | 25.175 | 57.653 | 99.428 | 1.00 | 24.29 |
| 771 | CG | TYR | A | 320 | 24.229 | 56.664 | 98.812 | 1.00 | 25.21 |
| 772 | CD1 | TYR | A | 320 | 24.639 | 55.378 | 98.426 | 1.00 | 20.61 |
| 773 | CE1 | TYR | A | 320 | 23.790 | 54.407 | 97.720 | 1.00 | 25.95 |
| 774 | CZ | TYR | A | 320 | 22.248 | 54.829 | 97.649 | 1.00 | 32.66 |
| 775 | OH | TYR | A | 320 | 21.501 | 53.957 | 97.050 | 1.00 | 24.66 |
| 776 | CE2 | TYR | A | 320 | 21.862 | 56.053 | 97.976 | 1.00 | 21.62 |
| 777 | CD2 | TYR | A | 320 | 22.843 | 57.025 | 98.602 | 1.00 | 26.81 |
| 778 | C | TYR | A | 320 | 25.805 | 56.288 | 101.364 | 1.00 | 26.52 |
| 779 | O | TYR | A | 320 | 26.968 | 55.930 | 101.180 | 1.00 | 28.23 |
| 780 | N | ASN | A | 321 | 24.941 | 55.500 | 101.837 | 1.00 | 25.11 |
| 781 | CA | ASN | A | 321 | 25.334 | 54.099 | 102.302 | 1.00 | 28.42 |
| 782 | CB | ASN | A | 321 | 24.383 | 53.766 | 103.482 | 1.00 | 28.30 |
| 783 | CG | ASN | A | 321 | 24.836 | 52.413 | 104.120 | 1.00 | 31.95 |
| 784 | OD1 | ASN | A | 321 | 25.389 | 51.540 | 103.415 | 1.00 | 31.12 |
| 785 | ND2 | ASN | A | 321 | 24.416 | 52.201 | 105.316 | 1.00 | 35.12 |
| 786 | C | ASN | A | 321 | 25.158 | 53.118 | 101.168 | 1.00 | 26.49 |
| 787 | O | ASN | A | 321 | 23.975 | 52.749 | 100.848 | 1.00 | 27.67 |
| 788 | N | HIS | A | 322 | 26.169 | 52.696 | 100.586 | 1.00 | 25.24 |
| 789 | CA | HIS | A | 322 | 26.128 | 51.761 | 99.497 | 1.00 | 27.12 |
| 790 | CB | HIS | A | 322 | 27.525 | 51.852 | 98.665 | 1.00 | 27.16 |
| 791 | CG | HIS | A | 322 | 27.558 | 53.165 | 97.858 | 1.00 | 29.49 |
| 792 | ND1 | HIS | A | 322 | 28.192 | 54.312 | 98.266 | 1.00 | 31.90 |
| 793 | CE1 | HIS | A | 322 | 28.037 | 55.289 | 97.434 | 1.00 | 26.71 |
| 794 | NE2 | HIS | A | 322 | 27.244 | 54.797 | 96.472 | 1.00 | 32.75 |
| 795 | CD2 | HIS | A | 322 | 26.937 | 53.461 | 96.704 | 1.00 | 26.25 |
| 796 | C | HIS | A | 322 | 26.004 | 50.314 | 99.969 | 1.00 | 31.64 |
| 797 | O | HIS | A | 322 | 25.774 | 49.416 | 99.115 | 1.00 | 32.39 |
| 798 | N | GLU | A | 323 | 26.013 | 50.052 | 101.259 | 1.00 | 27.45 |
| 799 | CA | GLU | A | 323 | 25.622 | 48.676 | 101.716 | 1.00 | 28.04 |
| 800 | CB | GLU | A | 323 | 26.154 | 48.463 | 103.239 | 1.00 | 30.66 |
| 801 | CG | GLU | A | 323 | 27.813 | 48.469 | 103.350 | 1.00 | 32.86 |
| 802 | CD | GLU | A | 323 | 28.260 | 48.311 | 104.726 | 1.00 | 37.39 |
| 803 | OE1 | GLU | A | 323 | 27.438 | 48.232 | 105.689 | 1.00 | 37.69 |
| 804 | OE2 | GLU | A | 323 | 29.409 | 48.392 | 104.899 | 1.00 | 40.31 |
| 805 | C | GLU | A | 323 | 24.244 | 48.515 | 101.810 | 1.00 | 30.19 |
| 806 | O | GLU | A | 323 | 23.667 | 47.456 | 101.420 | 1.00 | 31.86 |
| 807 | N | THR | A | 324 | 23.486 | 49.571 | 102.143 | 1.00 | 32.78 |
| 808 | CA | THR | A | 324 | 22.051 | 49.464 | 102.260 | 1.00 | 29.67 |
| 809 | CB | THR | A | 324 | 21.460 | 50.155 | 103.526 | 1.00 | 33.02 |
| 810 | OG1 | THR | A | 324 | 21.978 | 51.535 | 103.648 | 1.00 | 34.94 |
| 811 | CG2 | THR | A | 324 | 21.911 | 49.410 | 104.845 | 1.00 | 27.84 |
| 812 | C | THR | A | 324 | 21.376 | 50.117 | 101.095 | 1.00 | 34.32 |
| 813 | O | THR | A | 324 | 20.207 | 49.942 | 100.844 | 1.00 | 32.06 |
| 814 | N | GLU | A | 325 | 22.216 | 50.925 | 100.336 | 1.00 | 32.96 |
| 815 | CA | GLU | A | 325 | 21.551 | 51.720 | 99.197 | 1.00 | 30.82 |
| 816 | CB | GLU | A | 325 | 20.864 | 50.794 | 98.278 | 1.00 | 34.24 |
| 817 | CG | GLU | A | 325 | 21.772 | 49.654 | 97.698 | 1.00 | 40.03 |
| 818 | CD | GLU | A | 325 | 21.176 | 49.062 | 96.382 | 1.00 | 31.22 |
| 819 | OE1 | GLU | A | 325 | 21.435 | 49.641 | 95.380 | 1.00 | 32.46 |
| 820 | OE2 | GLU | A | 325 | 20.444 | 48.024 | 96.426 | 1.00 | 36.90 |
| 821 | C | GLU | A | 325 | 20.538 | 52.695 | 99.759 | 1.00 | 29.33 |
| 822 | O | GLU | A | 325 | 19.437 | 52.847 | 99.261 | 1.00 | 28.96 |
| 823 | N | CYS | A | 326 | 20.966 | 53.414 | 100.806 | 1.00 | 28.37 |
| 824 | CA | CYS | A | 326 | 20.204 | 54.402 | 101.402 | 1.00 | 33.99 |
| 825 | CB | CYS | A | 326 | 19.736 | 54.058 | 102.905 | 1.00 | 30.63 |
| 826 | SG | CYS | A | 326 | 18.387 | 52.785 | 102.879 | 1.00 | 37.75 |
| 827 | C | CYS | A | 326 | 20.850 | 55.806 | 101.400 | 1.00 | 33.40 |
| 828 | O | CYS | A | 326 | 22.066 | 55.910 | 101.595 | 1.00 | 29.29 |
| 829 | N | ILE | A | 327 | 20.013 | 56.857 | 101.324 | 1.00 | 30.10 |
| 830 | CA | ILE | A | 327 | 20.479 | 58.168 | 101.551 | 1.00 | 31.08 |
| 831 | CB | ILE | A | 327 | 19.648 | 59.263 | 100.922 | 1.00 | 35.00 |
| 832 | CG1 | ILE | A | 327 | 19.922 | 59.335 | 99.456 | 1.00 | 32.21 |
| 833 | CD1 | ILE | A | 327 | 18.984 | 60.151 | 98.568 | 1.00 | 38.04 |
| 834 | CG2 | ILE | A | 327 | 19.824 | 60.608 | 101.766 | 1.00 | 24.53 |
| 835 | C | ILE | A | 327 | 20.560 | 58.271 | 103.081 | 1.00 | 37.38 |
| 836 | O | ILE | A | 327 | 19.615 | 57.873 | 103.805 | 1.00 | 31.19 |
| 837 | N | THR | A | 328 | 21.698 | 58.782 | 103.646 | 1.00 | 34.91 |
| 838 | CA | THR | A | 328 | 21.649 | 58.850 | 105.022 | 1.00 | 38.61 |
| 839 | CB | THR | A | 328 | 22.849 | 57.874 | 105.657 | 1.00 | 42.84 |
| 840 | OG1 | THR | A | 328 | 24.111 | 58.423 | 105.337 | 1.00 | 47.71 |
| 841 | CG2 | THR | A | 328 | 22.794 | 56.504 | 105.135 | 1.00 | 36.12 |
| 842 | C | THR | A | 328 | 22.028 | 60.302 | 105.495 | 1.00 | 45.59 |
| 843 | O | THR | A | 328 | 22.867 | 60.868 | 104.936 | 1.00 | 47.31 |
| 844 | N | PHE | A | 329 | 21.320 | 60.862 | 106.490 | 1.00 | 45.91 |
| 845 | CA | PHE | A | 329 | 21.630 | 62.013 | 107.122 | 1.00 | 48.86 |
| 846 | CB | PHE | A | 329 | 20.374 | 62.537 | 107.626 | 1.00 | 54.49 |
| 847 | CG | PHE | A | 329 | 20.375 | 64.083 | 107.850 | 1.00 | 57.05 |
| 848 | CD1 | PHE | A | 329 | 20.773 | 64.668 | 109.142 | 1.00 | 58.41 |
| 849 | CE1 | PHE | A | 329 | 20.836 | 65.959 | 109.330 | 1.00 | 53.09 |
| 850 | CZ | PHE | A | 329 | 20.472 | 66.792 | 108.224 | 1.00 | 58.19 |
| 851 | CE2 | PHE | A | 329 | 20.006 | 66.295 | 107.089 | 1.00 | 59.51 |
| 852 | CD2 | PHE | A | 329 | 20.038 | 64.861 | 106.864 | 1.00 | 59.08 |
| 853 | C | PHE | A | 329 | 22.544 | 61.989 | 108.310 | 1.00 | 56.52 |
| 854 | O | PHE | A | 329 | 23.478 | 62.853 | 108.405 | 1.00 | 55.51 |
| 855 | N | LEU | A | 330 | 22.599 | 60.870 | 109.145 | 1.00 | 60.17 |
| 856 | CA | LEU | A | 330 | 23.569 | 60.696 | 110.259 | 1.00 | 60.42 |
| 857 | CB | LEU | A | 330 | 22.931 | 61.142 | 111.628 | 1.00 | 65.77 |
| 858 | CG | LEU | A | 330 | 22.352 | 62.480 | 111.953 | 1.00 | 69.35 |
| 859 | CD1 | LEU | A | 330 | 21.234 | 62.451 | 113.038 | 1.00 | 58.83 |
| 860 | CD2 | LEU | A | 330 | 23.379 | 63.514 | 112.265 | 1.00 | 68.98 |
| 861 | C | LEU | A | 330 | 23.585 | 59.283 | 110.547 | 1.00 | 65.60 |
| 862 | O | LEU | A | 330 | 23.434 | 58.522 | 109.643 | 1.00 | 74.89 |
| 863 | N | LYS | A | 331 | 23.914 | 58.782 | 111.766 | 1.00 | 67.54 |
| 864 | CA | LYS | A | 331 | 24.161 | 57.267 | 112.064 | 1.00 | 65.86 |
| 865 | CB | LYS | A | 331 | 24.425 | 56.954 | 113.509 | 1.00 | 65.68 |
| 866 | CG | LYS | A | 331 | 24.386 | 55.393 | 113.819 | 1.00 | 65.19 |
| 867 | CD | LYS | A | 331 | 25.843 | 54.758 | 113.844 | 1.00 | 71.83 |
| 868 | CE | LYS | A | 331 | 25.926 | 53.704 | 112.636 | 1.00 | 75.49 |
| 869 | NZ | LYS | A | 331 | 27.122 | 52.694 | 112.810 | 1.00 | 69.11 |
| 870 | C | LYS | A | 331 | 22.844 | 56.568 | 111.679 | 1.00 | 68.90 |
| 871 | O | LYS | A | 331 | 22.655 | 55.556 | 110.771 | 1.00 | 73.86 |
| 872 | N | ASP | A | 332 | 21.791 | 57.094 | 112.183 | 1.00 | 65.17 |
| 873 | CA | ASP | A | 332 | 20.632 | 56.645 | 111.561 | 1.00 | 60.60 |
| 874 | CB | ASP | A | 332 | 19.713 | 56.336 | 112.511 | 1.00 | 69.63 |
| 875 | CG | ASP | A | 332 | 18.518 | 56.016 | 111.882 | 1.00 | 75.01 |
| 876 | OD1 | ASP | A | 332 | 18.294 | 54.751 | 111.433 | 1.00 | 71.44 |
| 877 | OD2 | ASP | A | 332 | 17.661 | 57.004 | 111.855 | 1.00 | 81.67 |
| 878 | C | ASP | A | 332 | 20.115 | 57.971 | 110.877 | 1.00 | 62.41 |
| 879 | O | ASP | A | 332 | 21.027 | 58.937 | 110.472 | 1.00 | 59.25 |
| 880 | N | PHE | A | 333 | 18.885 | 57.972 | 110.615 | 1.00 | 55.78 |
| 881 | CA | PHE | A | 333 | 18.411 | 59.028 | 109.692 | 1.00 | 53.49 |
| 882 | CB | PHE | A | 333 | 18.841 | 60.348 | 110.084 | 1.00 | 53.02 |
| 883 | CG | PHE | A | 333 | 18.261 | 60.740 | 111.480 | 1.00 | 65.51 |
| 884 | CD1 | PHE | A | 333 | 16.924 | 61.279 | 111.645 | 1.00 | 60.74 |
| 885 | CE1 | PHE | A | 333 | 16.361 | 61.547 | 112.836 | 1.00 | 64.61 |
| 886 | CZ | PHE | A | 333 | 17.014 | 61.292 | 114.094 | 1.00 | 63.64 |
| 887 | CE2 | PHE | A | 333 | 18.384 | 60.674 | 113.944 | 1.00 | 66.77 |
| 888 | CD2 | PHE | A | 333 | 18.945 | 60.322 | 112.619 | 1.00 | 60.92 |
| 889 | C | PHE | A | 333 | 18.817 | 58.523 | 108.320 | 1.00 | 50.37 |
| 890 | O | PHE | A | 333 | 19.720 | 59.126 | 107.757 | 1.00 | 53.42 |
| 891 | N | THR | A | 334 | 18.260 | 57.449 | 107.859 | 1.00 | 44.86 |
| 892 | CA | THR | A | 334 | 18.637 | 56.862 | 106.598 | 1.00 | 50.38 |
| 893 | CB | THR | A | 334 | 19.446 | 55.608 | 106.621 | 1.00 | 40.77 |
| 894 | OG1 | THR | A | 334 | 18.592 | 54.729 | 107.063 | 1.00 | 49.86 |
| 895 | CG2 | THR | A | 334 | 20.279 | 55.736 | 107.619 | 1.00 | 45.74 |
| 896 | C | THR | A | 334 | 17.326 | 56.680 | 105.822 | 1.00 | 45.06 |
| 897 | O | THR | A | 334 | 16.483 | 56.488 | 106.505 | 1.00 | 42.68 |
| 898 | N | TYR | A | 335 | 17.296 | 56.814 | 104.413 | 1.00 | 38.65 |
| 899 | CA | TYR | A | 335 | 16.190 | 56.725 | 103.734 | 1.00 | 34.67 |
| 900 | CB | TYR | A | 335 | 15.626 | 58.253 | 103.429 | 1.00 | 35.54 |
| 901 | CG | TYR | A | 335 | 15.684 | 59.043 | 104.542 | 1.00 | 36.77 |
| 902 | CD1 | TYR | A | 335 | 14.552 | 58.964 | 105.658 | 1.00 | 44.72 |
| 903 | CE1 | TYR | A | 335 | 14.500 | 59.789 | 106.758 | 1.00 | 36.72 |
| 904 | CZ | TYR | A | 335 | 15.590 | 60.509 | 107.070 | 1.00 | 38.85 |
| 905 | OH | TYR | A | 335 | 15.901 | 61.059 | 108.257 | 1.00 | 43.46 |
| 906 | CE2 | TYR | A | 335 | 16.823 | 60.402 | 106.209 | 1.00 | 48.91 |
| 907 | CD2 | TYR | A | 335 | 16.763 | 59.725 | 104.854 | 1.00 | 38.14 |
| 908 | C | TYR | A | 335 | 16.345 | 56.075 | 102.417 | 1.00 | 37.64 |
| 909 | O | TYR | A | 335 | 17.182 | 56.316 | 101.689 | 1.00 | 36.38 |
| 910 | N | SER | A | 336 | 15.301 | 55.219 | 102.027 | 1.00 | 39.92 |
| 911 | CA | SER | A | 336 | 15.232 | 54.471 | 100.786 | 1.00 | 36.88 |
| 912 | CB | SER | A | 336 | 14.536 | 53.093 | 101.117 | 1.00 | 35.99 |
| 913 | OG | SER | A | 336 | 13.154 | 53.432 | 101.130 | 1.00 | 42.46 |
| 914 | C | SER | A | 336 | 14.454 | 55.251 | 99.867 | 1.00 | 33.88 |
| 915 | O | SER | A | 336 | 13.945 | 56.283 | 100.248 | 1.00 | 37.82 |
| 916 | N | LYS | A | 337 | 14.434 | 54.855 | 98.650 | 1.00 | 41.15 |
| 917 | CA | LYS | A | 337 | 13.616 | 55.428 | 97.582 | 1.00 | 41.23 |
| 918 | CB | LYS | A | 337 | 13.529 | 54.657 | 96.356 | 1.00 | 36.95 |
| 919 | CG | LYS | A | 337 | 15.009 | 54.557 | 95.596 | 1.00 | 45.49 |
| 920 | CD | LYS | A | 337 | 14.894 | 54.356 | 94.258 | 1.00 | 41.74 |
| 921 | CE | LYS | A | 337 | 16.307 | 54.149 | 93.449 | 1.00 | 34.02 |
| 922 | NZ | LYS | A | 337 | 16.044 | 54.036 | 92.021 | 1.00 | 36.78 |
| 923 | C | LYS | A | 337 | 12.189 | 55.459 | 98.093 | 1.00 | 48.52 |
| 924 | O | LYS | A | 337 | 11.417 | 56.559 | 98.072 | 1.00 | 45.96 |
| 925 | N | ASP | A | 338 | 11.775 | 54.388 | 98.698 | 1.00 | 47.64 |
| 926 | CA | ASP | A | 338 | 10.467 | 54.351 | 99.250 | 1.00 | 46.38 |
| 927 | CB | ASP | A | 338 | 10.281 | 52.998 | 99.993 | 1.00 | 53.22 |
| 928 | CG | ASP | A | 338 | 8.789 | 52.612 | 100.012 | 1.00 | 64.06 |
| 929 | OD1 | ASP | A | 338 | 8.167 | 52.411 | 98.833 | 1.00 | 64.38 |
| 930 | OD2 | ASP | A | 338 | 8.237 | 52.624 | 101.173 | 1.00 | 66.10 |
| 931 | C | ASP | A | 338 | 10.083 | 55.401 | 100.113 | 1.00 | 46.94 |
| 932 | O | ASP | A | 338 | 8.913 | 55.975 | 99.972 | 1.00 | 45.82 |
| 933 | N | ASP | A | 339 | 10.932 | 55.731 | 101.049 | 1.00 | 47.85 |
| 934 | CA | ASP | A | 339 | 10.713 | 56.747 | 101.948 | 1.00 | 47.16 |
| 935 | CB | ASP | A | 339 | 11.729 | 56.874 | 103.045 | 1.00 | 49.83 |
| 936 | CG | ASP | A | 339 | 11.845 | 55.620 | 103.954 | 1.00 | 60.04 |
| 937 | OD1 | ASP | A | 339 | 10.791 | 55.173 | 104.487 | 1.00 | 53.48 |
| 938 | OD2 | ASP | A | 339 | 13.021 | 55.140 | 104.230 | 1.00 | 52.58 |
| 939 | C | ASP | A | 339 | 10.476 | 58.146 | 101.295 | 1.00 | 53.26 |
| 940 | O | ASP | A | 339 | 9.605 | 58.969 | 101.768 | 1.00 | 55.83 |
| 941 | N | PHE | A | 340 | 11.191 | 58.416 | 100.275 | 1.00 | 54.63 |
| 942 | CA | PHE | A | 340 | 11.047 | 59.664 | 99.572 | 1.00 | 55.04 |
| 943 | CB | PHE | A | 340 | 12.235 | 59.822 | 98.583 | 1.00 | 48.17 |
| 944 | CG | PHE | A | 340 | 13.503 | 60.320 | 99.262 | 1.00 | 43.47 |
| 945 | CD1 | PHE | A | 340 | 13.622 | 61.684 | 99.643 | 1.00 | 46.40 |
| 946 | CE1 | PHE | A | 340 | 14.746 | 62.234 | 100.336 | 1.00 | 37.14 |
| 947 | CZ | PHE | A | 340 | 15.703 | 61.253 | 100.757 | 1.00 | 43.67 |
| 948 | CE2 | PHE | A | 340 | 15.586 | 59.964 | 100.282 | 1.00 | 40.75 |
| 949 | CD2 | PHE | A | 340 | 14.458 | 59.505 | 99.639 | 1.00 | 39.97 |
| 950 | C | PHE | A | 340 | 9.683 | 59.640 | 98.977 | 1.00 | 57.44 |
| 951 | O | PHE | A | 340 | 9.039 | 60.788 | 98.918 | 1.00 | 60.00 |
| 952 | N | ALA | A | 341 | 9.364 | 58.561 | 98.326 | 1.00 | 52.40 |
| 953 | CA | ALA | A | 341 | 8.080 | 58.384 | 97.615 | 1.00 | 58.89 |
| 954 | CB | ALA | A | 341 | 8.005 | 56.891 | 97.006 | 1.00 | 58.65 |
| 955 | C | ALA | A | 341 | 6.931 | 58.616 | 98.619 | 1.00 | 60.38 |
| 956 | O | ALA | A | 341 | 6.067 | 59.406 | 98.483 | 1.00 | 60.21 |
| 957 | N | ARG | A | 342 | 7.087 | 58.039 | 99.749 | 1.00 | 63.11 |
| 958 | CA | ARG | A | 342 | 6.030 | 58.242 | 100.768 | 1.00 | 64.22 |
| 959 | CB | ARG | A | 342 | 6.255 | 57.371 | 101.965 | 1.00 | 61.32 |
| 960 | CG | ARG | A | 342 | 5.973 | 55.945 | 102.077 | 1.00 | 62.09 |
| 961 | CD | ARG | A | 342 | 6.818 | 55.614 | 103.454 | 1.00 | 75.15 |
| 962 | NE | ARG | A | 342 | 6.826 | 54.231 | 103.990 | 1.00 | 81.85 |
| 963 | CZ | ARG | A | 342 | 7.986 | 53.453 | 103.861 | 1.00 | 90.20 |
| 964 | NH1 | ARG | A | 342 | 9.079 | 53.917 | 103.227 | 1.00 | 76.80 |
| 965 | NH2 | ARG | A | 342 | 8.054 | 52.196 | 104.362 | 1.00 | 94.52 |
| 966 | C | ARG | A | 342 | 5.874 | 59.675 | 101.260 | 1.00 | 65.87 |
| 967 | O | ARG | A | 342 | 4.717 | 60.059 | 101.953 | 1.00 | 60.63 |
| 968 | N | ALA | A | 343 | 6.872 | 60.483 | 100.965 | 1.00 | 60.17 |
| 969 | CA | ALA | A | 343 | 6.584 | 61.826 | 101.407 | 1.00 | 57.27 |
| 970 | CB | ALA | A | 343 | 7.909 | 62.466 | 101.859 | 1.00 | 57.16 |
| 971 | C | ALA | A | 343 | 5.916 | 62.603 | 100.352 | 1.00 | 56.52 |
| 972 | O | ALA | A | 343 | 5.756 | 63.831 | 100.516 | 1.00 | 62.82 |
| 973 | N | GLY | A | 344 | 5.628 | 62.014 | 99.187 | 1.00 | 57.55 |
| 974 | CA | GLY | A | 344 | 4.963 | 62.749 | 98.095 | 1.00 | 57.86 |
| 975 | C | GLY | A | 344 | 5.934 | 63.355 | 97.042 | 1.00 | 65.75 |
| 976 | O | GLY | A | 344 | 5.547 | 64.325 | 96.335 | 1.00 | 66.13 |
| 977 | N | LEU | A | 345 | 7.154 | 62.667 | 96.692 | 1.00 | 62.47 |
| 978 | CA | LEU | A | 345 | 7.996 | 63.147 | 95.694 | 1.00 | 55.91 |
| 979 | CB | LEU | A | 345 | 9.295 | 63.261 | 96.392 | 1.00 | 61.92 |
| 980 | CG | LEU | A | 345 | 9.344 | 63.843 | 97.823 | 1.00 | 68.77 |
| 981 | CD1 | LEU | A | 345 | 10.803 | 63.704 | 98.515 | 1.00 | 68.43 |
| 982 | CD2 | LEU | A | 345 | 8.863 | 65.212 | 97.921 | 1.00 | 59.11 |
| 983 | C | LEU | A | 345 | 7.999 | 62.433 | 94.515 | 1.00 | 49.37 |
| 984 | O | LEU | A | 345 | 7.821 | 61.282 | 94.442 | 1.00 | 53.23 |
| 985 | N | GLN | A | 346 | 8.221 | 63.061 | 93.411 | 1.00 | 53.41 |
| 986 | CA | GLN | A | 346 | 8.260 | 62.430 | 92.052 | 1.00 | 55.12 |
| 987 | CB | GLN | A | 346 | 8.620 | 63.542 | 90.955 | 1.00 | 61.52 |
| 988 | CG | GLN | A | 346 | 7.597 | 64.461 | 90.620 | 1.00 | 71.32 |
| 989 | CD | GLN | A | 346 | 8.231 | 65.855 | 89.889 | 1.00 | 69.01 |
| 990 | OE1 | GLN | A | 346 | 9.433 | 66.147 | 89.952 | 1.00 | 71.20 |
| 991 | NE2 | GLN | A | 346 | 7.411 | 66.614 | 89.181 | 1.00 | 81.10 |
| 992 | C | GLN | A | 346 | 9.301 | 61.292 | 92.036 | 1.00 | 55.12 |
| 993 | O | GLN | A | 346 | 10.375 | 61.517 | 92.703 | 1.00 | 55.52 |
| 994 | N | VAL | A | 347 | 9.097 | 60.428 | 91.127 | 1.00 | 54.09 |
| 995 | CA | VAL | A | 347 | 10.096 | 59.503 | 90.928 | 1.00 | 58.16 |
| 996 | CB | VAL | A | 347 | 9.468 | 58.181 | 90.310 | 1.00 | 58.60 |
| 997 | CG1 | VAL | A | 347 | 9.789 | 58.039 | 88.819 | 1.00 | 61.76 |
| 998 | CG2 | VAL | A | 347 | 9.978 | 57.044 | 91.036 | 1.00 | 60.39 |
| 999 | C | VAL | A | 347 | 11.131 | 60.187 | 90.012 | 1.00 | 55.68 |
| 1000 | O | VAL | A | 347 | 12.265 | 59.797 | 89.921 | 1.00 | 49.89 |
| 1001 | N | GLU | A | 348 | 10.736 | 61.178 | 89.370 | 1.00 | 52.48 |
| 1002 | CA | GLU | A | 348 | 11.492 | 61.768 | 88.390 | 1.00 | 49.37 |
| 1003 | CB | GLU | A | 348 | 10.663 | 62.783 | 87.401 | 1.00 | 49.65 |
| 1004 | CG | GLU | A | 348 | 8.990 | 62.445 | 87.511 | 1.00 | 63.05 |
| 1005 | CD | GLU | A | 348 | 8.496 | 61.453 | 86.590 | 1.00 | 70.49 |
| 1006 | OE1 | GLU | A | 348 | 8.720 | 60.141 | 86.764 | 1.00 | 81.28 |
| 1007 | OE2 | GLU | A | 348 | 7.971 | 61.742 | 85.393 | 1.00 | 83.18 |
| 1008 | C | GLU | A | 348 | 12.614 | 62.639 | 89.157 | 1.00 | 48.56 |
| 1009 | O | GLU | A | 348 | 13.661 | 63.034 | 88.442 | 1.00 | 46.22 |
| 1010 | N | PHE | A | 349 | 12.458 | 62.792 | 90.386 | 1.00 | 41.51 |
| 1011 | CA | PHE | A | 349 | 13.435 | 63.585 | 91.173 | 1.00 | 42.56 |
| 1012 | CB | PHE | A | 349 | 12.722 | 64.383 | 92.160 | 1.00 | 35.26 |
| 1013 | CG | PHE | A | 349 | 13.570 | 64.980 | 93.173 | 1.00 | 38.81 |
| 1014 | CD1 | PHE | A | 349 | 14.670 | 65.775 | 92.802 | 1.00 | 36.08 |
| 1015 | CE1 | PHE | A | 349 | 15.522 | 66.444 | 93.729 | 1.00 | 31.95 |
| 1016 | CZ | PHE | A | 349 | 15.393 | 66.088 | 95.047 | 1.00 | 37.40 |
| 1017 | CE2 | PHE | A | 349 | 14.315 | 65.302 | 95.471 | 1.00 | 38.65 |
| 1018 | CD2 | PHE | A | 349 | 13.371 | 64.784 | 94.510 | 1.00 | 37.78 |
| 1019 | C | PHE | A | 349 | 14.222 | 62.503 | 92.001 | 1.00 | 40.40 |
| 1020 | O | PHE | A | 349 | 15.470 | 62.477 | 91.940 | 1.00 | 40.17 |
| 1021 | N | ILE | A | 350 | 13.471 | 61.555 | 92.632 | 1.00 | 39.12 |
| 1022 | CA | ILE | A | 350 | 14.005 | 60.622 | 93.405 | 1.00 | 43.56 |
| 1023 | CB | ILE | A | 350 | 12.895 | 59.747 | 93.968 | 1.00 | 44.06 |
| 1024 | CG1 | ILE | A | 350 | 12.078 | 60.539 | 94.821 | 1.00 | 42.70 |
| 1025 | CD1 | ILE | A | 350 | 10.586 | 59.645 | 95.281 | 1.00 | 38.43 |
| 1026 | CG2 | ILE | A | 350 | 13.427 | 58.485 | 94.759 | 1.00 | 29.85 |
| 1027 | C | ILE | A | 350 | 15.130 | 59.579 | 92.719 | 1.00 | 38.73 |
| 1028 | O | ILE | A | 350 | 16.126 | 59.294 | 93.270 | 1.00 | 36.16 |
| 1029 | N | ASN | A | 351 | 14.715 | 59.060 | 91.562 | 1.00 | 33.28 |
| 1030 | CA | ASN | A | 351 | 15.622 | 58.280 | 90.866 | 1.00 | 37.86 |
| 1031 | CB | ASN | A | 351 | 15.056 | 57.716 | 89.573 | 1.00 | 33.38 |
| 1032 | CG | ASN | A | 351 | 14.137 | 56.437 | 89.867 | 1.00 | 42.08 |
| 1033 | OD1 | ASN | A | 351 | 14.375 | 55.739 | 90.755 | 1.00 | 42.74 |
| 1034 | ND2 | ASN | A | 351 | 13.142 | 56.144 | 88.889 | 1.00 | 40.71 |
| 1035 | C | ASN | A | 351 | 16.949 | 58.876 | 90.592 | 1.00 | 40.02 |
| 1036 | O | ASN | A | 351 | 17.994 | 58.406 | 90.937 | 1.00 | 36.40 |
| 1037 | N | PRO | A | 352 | 16.914 | 60.098 | 90.039 | 1.00 | 39.55 |
| 1038 | CA | PRO | A | 352 | 18.128 | 60.755 | 89.704 | 1.00 | 38.11 |
| 1039 | CB | PRO | A | 352 | 17.756 | 61.978 | 88.896 | 1.00 | 36.01 |
| 1040 | CG | PRO | A | 352 | 16.469 | 61.625 | 88.308 | 1.00 | 43.28 |
| 1041 | CD | PRO | A | 352 | 15.720 | 60.797 | 89.589 | 1.00 | 37.85 |
| 1042 | C | PRO | A | 352 | 19.082 | 61.164 | 90.906 | 1.00 | 32.74 |
| 1043 | O | PRO | A | 352 | 20.306 | 60.952 | 90.779 | 1.00 | 30.16 |
| 1044 | N | ILE | A | 353 | 18.538 | 61.191 | 92.104 | 1.00 | 31.77 |
| 1045 | CA | ILE | A | 353 | 19.448 | 61.624 | 93.177 | 1.00 | 32.21 |
| 1046 | CB | ILE | A | 353 | 18.830 | 62.379 | 94.299 | 1.00 | 34.16 |
| 1047 | CG1 | ILE | A | 353 | 17.761 | 61.547 | 95.059 | 1.00 | 28.49 |
| 1048 | CD1 | ILE | A | 353 | 17.200 | 62.312 | 96.280 | 1.00 | 25.50 |
| 1049 | CG2 | ILE | A | 353 | 18.223 | 63.880 | 93.727 | 1.00 | 40.63 |
| 1050 | C | ILE | A | 353 | 20.110 | 60.396 | 93.634 | 1.00 | 35.07 |
| 1051 | O | ILE | A | 353 | 21.247 | 60.453 | 94.107 | 1.00 | 33.62 |
| 1052 | N | PHE | A | 354 | 19.363 | 59.302 | 93.704 | 1.00 | 33.04 |
| 1053 | CA | PHE | A | 354 | 19.973 | 57.995 | 94.111 | 1.00 | 37.01 |
| 1054 | CB | PHE | A | 354 | 18.983 | 56.887 | 94.218 | 1.00 | 33.54 |
| 1055 | CG | PHE | A | 354 | 18.282 | 56.918 | 95.538 | 1.00 | 34.07 |
| 1056 | CD1 | PHE | A | 354 | 17.298 | 57.826 | 95.769 | 1.00 | 29.36 |
| 1057 | CE1 | PHE | A | 354 | 16.642 | 57.859 | 97.051 | 1.00 | 32.83 |
| 1058 | CZ | PHE | A | 354 | 17.082 | 56.950 | 98.051 | 1.00 | 35.19 |
| 1059 | CE2 | PHE | A | 354 | 18.010 | 56.095 | 97.810 | 1.00 | 36.74 |
| 1060 | CD2 | PHE | A | 354 | 18.653 | 56.011 | 96.491 | 1.00 | 34.92 |
| 1061 | C | PHE | A | 354 | 21.034 | 57.537 | 92.983 | 1.00 | 36.17 |
| 1062 | O | PHE | A | 354 | 22.079 | 57.025 | 93.330 | 1.00 | 38.46 |
| 1063 | N | GLU | A | 355 | 20.729 | 57.777 | 91.739 | 1.00 | 32.99 |
| 1064 | CA | GLU | A | 355 | 21.603 | 57.570 | 90.635 | 1.00 | 31.95 |
| 1065 | CB | GLU | A | 355 | 20.849 | 57.683 | 89.349 | 1.00 | 30.61 |
| 1066 | CG | GLU | A | 355 | 21.238 | 56.779 | 88.200 | 1.00 | 45.49 |
| 1067 | CD | GLU | A | 355 | 21.419 | 55.196 | 88.539 | 1.00 | 48.90 |
| 1068 | OE1 | GLU | A | 355 | 20.531 | 54.439 | 89.257 | 1.00 | 44.46 |
| 1069 | OE2 | GLU | A | 355 | 22.571 | 54.858 | 88.301 | 1.00 | 47.34 |
| 1070 | C | GLU | A | 355 | 22.846 | 58.352 | 90.660 | 1.00 | 29.03 |
| 1071 | O | GLU | A | 355 | 23.939 | 57.950 | 90.316 | 1.00 | 34.92 |
| 1072 | N | PHE | A | 356 | 22.700 | 59.587 | 91.085 | 1.00 | 29.34 |
| 1073 | CA | PHE | A | 356 | 23.879 | 60.438 | 91.159 | 1.00 | 28.44 |
| 1074 | CB | PHE | A | 356 | 23.511 | 62.025 | 91.372 | 1.00 | 24.84 |
| 1075 | CG | PHE | A | 356 | 24.666 | 62.810 | 91.513 | 1.00 | 26.82 |
| 1076 | CD1 | PHE | A | 356 | 25.398 | 63.241 | 90.389 | 1.00 | 20.56 |
| 1077 | CE1 | PHE | A | 356 | 26.417 | 64.033 | 90.504 | 1.00 | 22.91 |
| 1078 | CZ | PHE | A | 356 | 26.735 | 64.591 | 91.746 | 1.00 | 22.03 |
| 1079 | CE2 | PHE | A | 356 | 26.001 | 64.229 | 92.833 | 1.00 | 24.05 |
| 1080 | CD2 | PHE | A | 356 | 24.978 | 63.331 | 92.731 | 1.00 | 21.52 |
| 1081 | C | PHE | A | 356 | 24.779 | 59.836 | 92.315 | 1.00 | 24.60 |
| 1082 | O | PHE | A | 356 | 25.987 | 59.838 | 92.144 | 1.00 | 26.66 |
| 1083 | N | SER | A | 357 | 24.215 | 59.596 | 93.391 | 1.00 | 20.88 |
| 1084 | CA | SER | A | 357 | 25.002 | 59.033 | 94.542 | 1.00 | 26.21 |
| 1085 | CB | SER | A | 357 | 24.087 | 58.603 | 95.676 | 1.00 | 26.96 |
| 1086 | OG | SER | A | 357 | 23.475 | 59.777 | 96.266 | 1.00 | 35.03 |
| 1087 | C | SER | A | 357 | 25.747 | 57.742 | 94.006 | 1.00 | 27.43 |
| 1088 | O | SER | A | 357 | 26.969 | 57.600 | 94.360 | 1.00 | 26.40 |
| 1089 | N | ARG | A | 358 | 25.134 | 56.946 | 93.210 | 1.00 | 22.54 |
| 1090 | CA | ARG | A | 358 | 25.793 | 55.760 | 92.607 | 1.00 | 28.46 |
| 1091 | CB | ARG | A | 358 | 24.843 | 54.824 | 91.803 | 1.00 | 27.12 |
| 1092 | CG | ARG | A | 358 | 23.697 | 54.269 | 92.620 | 1.00 | 31.43 |
| 1093 | CD | ARG | A | 358 | 22.934 | 53.389 | 91.651 | 1.00 | 33.88 |
| 1094 | NE | ARG | A | 358 | 23.872 | 52.537 | 91.013 | 1.00 | 43.04 |
| 1095 | CZ | ARG | A | 358 | 23.589 | 51.618 | 89.966 | 1.00 | 46.48 |
| 1096 | NH1 | ARG | A | 358 | 22.381 | 51.806 | 89.350 | 1.00 | 41.56 |
| 1097 | NH2 | ARG | A | 358 | 24.554 | 50.764 | 89.510 | 1.00 | 38.99 |
| 1098 | C | ARG | A | 358 | 26.993 | 56.041 | 91.707 | 1.00 | 25.01 |
| 1099 | O | ARG | A | 358 | 28.092 | 55.610 | 91.893 | 1.00 | 29.34 |
| 1100 | N | ALA | A | 359 | 26.747 | 56.925 | 90.736 | 1.00 | 27.15 |
| 1101 | CA | ALA | A | 359 | 27.774 | 57.289 | 89.826 | 1.00 | 25.84 |
| 1102 | CB | ALA | A | 359 | 27.068 | 58.411 | 88.707 | 1.00 | 29.99 |
| 1103 | C | ALA | A | 359 | 28.951 | 58.049 | 90.486 | 1.00 | 28.51 |
| 1104 | O | ALA | A | 359 | 30.109 | 57.815 | 90.086 | 1.00 | 33.57 |
| 1105 | N | MET | A | 360 | 28.681 | 58.972 | 91.463 | 1.00 | 24.05 |
| 1106 | CA | MET | A | 360 | 29.724 | 59.525 | 92.232 | 1.00 | 27.60 |
| 1107 | CB | MET | A | 360 | 29.160 | 60.535 | 93.321 | 1.00 | 23.55 |
| 1108 | CG | MET | A | 360 | 28.826 | 62.038 | 92.888 | 1.00 | 21.34 |
| 1109 | SD | MET | A | 360 | 29.972 | 62.957 | 91.915 | 1.00 | 30.06 |
| 1110 | CE | MET | A | 360 | 31.260 | 63.327 | 93.225 | 1.00 | 27.47 |
| 1111 | C | MET | A | 360 | 30.659 | 58.521 | 92.938 | 1.00 | 26.47 |
| 1112 | O | MET | A | 360 | 31.912 | 58.707 | 92.962 | 1.00 | 24.69 |
| 1113 | N | ARG | A | 361 | 30.082 | 57.533 | 93.516 | 1.00 | 25.42 |
| 1114 | CA | ARG | A | 361 | 30.820 | 56.539 | 94.212 | 1.00 | 27.69 |
| 1115 | CB | ARG | A | 361 | 29.893 | 55.416 | 94.765 | 1.00 | 25.52 |
| 1116 | CG | ARG | A | 361 | 30.639 | 54.373 | 95.441 | 1.00 | 26.92 |
| 1117 | CD | ARG | A | 361 | 31.653 | 54.932 | 96.590 | 1.00 | 30.50 |
| 1118 | NE | ARG | A | 361 | 32.360 | 53.943 | 97.240 | 1.00 | 30.09 |
| 1119 | CZ | ARG | A | 361 | 33.541 | 53.386 | 96.823 | 1.00 | 34.94 |
| 1120 | NH1 | ARG | A | 361 | 34.182 | 53.879 | 95.769 | 1.00 | 27.55 |
| 1121 | NH2 | ARG | A | 361 | 34.015 | 52.393 | 97.463 | 1.00 | 31.71 |
| 1122 | C | ARG | A | 361 | 31.817 | 55.821 | 93.244 | 1.00 | 29.95 |
| 1123 | O | ARG | A | 361 | 32.842 | 55.424 | 93.659 | 1.00 | 32.18 |
| 1124 | N | ARG | A | 362 | 31.499 | 55.806 | 91.915 | 1.00 | 29.17 |
| 1125 | CA | ARG | A | 362 | 32.382 | 55.116 | 90.952 | 1.00 | 32.80 |
| 1126 | CB | ARG | A | 362 | 31.749 | 55.079 | 89.592 | 1.00 | 35.25 |
| 1127 | CG | ARG | A | 362 | 30.773 | 53.820 | 89.351 | 1.00 | 44.57 |
| 1128 | CD | ARG | A | 362 | 29.771 | 53.880 | 88.402 | 1.00 | 41.11 |
| 1129 | NE | ARG | A | 362 | 30.318 | 54.533 | 87.230 | 1.00 | 62.15 |
| 1130 | CZ | ARG | A | 362 | 31.219 | 53.932 | 86.299 | 1.00 | 56.43 |
| 1131 | NH1 | ARG | A | 362 | 31.790 | 52.909 | 86.664 | 1.00 | 76.32 |
| 1132 | NH2 | ARG | A | 362 | 31.752 | 54.433 | 85.291 | 1.00 | 58.84 |
| 1133 | C | ARG | A | 362 | 33.715 | 55.888 | 90.842 | 1.00 | 38.96 |
| 1134 | O | ARG | A | 362 | 34.742 | 55.280 | 90.453 | 1.00 | 34.44 |
| 1135 | N | LEU | A | 363 | 33.711 | 57.213 | 91.173 | 1.00 | 35.42 |
| 1136 | CA | LEU | A | 363 | 34.936 | 57.932 | 91.121 | 1.00 | 33.23 |
| 1137 | CB | LEU | A | 363 | 34.569 | 59.348 | 90.917 | 1.00 | 31.74 |
| 1138 | CG | LEU | A | 363 | 33.942 | 59.767 | 89.493 | 1.00 | 42.89 |
| 1139 | CD1 | LEU | A | 363 | 33.687 | 61.239 | 89.400 | 1.00 | 41.37 |
| 1140 | CD2 | LEU | A | 363 | 34.696 | 59.249 | 88.506 | 1.00 | 40.65 |
| 1141 | C | LEU | A | 363 | 35.862 | 57.697 | 92.410 | 1.00 | 36.34 |
| 1142 | O | LEU | A | 363 | 36.930 | 58.187 | 92.423 | 1.00 | 35.01 |
| 1143 | N | GLY | A | 364 | 35.343 | 57.163 | 93.426 | 1.00 | 32.97 |
| 1144 | CA | GLY | A | 364 | 36.041 | 56.985 | 94.595 | 1.00 | 30.77 |
| 1145 | C | GLY | A | 364 | 36.681 | 58.169 | 95.246 | 1.00 | 35.17 |
| 1146 | O | GLY | A | 364 | 37.993 | 58.033 | 95.528 | 1.00 | 33.06 |
| 1147 | N | LEU | A | 365 | 36.021 | 59.279 | 95.390 | 1.00 | 29.40 |
| 1148 | CA | LEU | A | 365 | 36.470 | 60.445 | 95.940 | 1.00 | 34.74 |
| 1149 | CB | LEU | A | 365 | 35.574 | 61.723 | 95.760 | 1.00 | 29.56 |
| 1150 | CG | LEU | A | 365 | 34.993 | 62.073 | 94.350 | 1.00 | 33.44 |
| 1151 | CD1 | LEU | A | 365 | 34.585 | 63.514 | 94.294 | 1.00 | 34.57 |
| 1152 | CD2 | LEU | A | 365 | 35.587 | 61.702 | 93.139 | 1.00 | 32.84 |
| 1153 | C | LEU | A | 365 | 36.716 | 60.411 | 97.557 | 1.00 | 35.27 |
| 1154 | O | LEU | A | 365 | 35.915 | 59.695 | 98.275 | 1.00 | 33.84 |
| 1155 | N | ASP | A | 366 | 37.723 | 61.044 | 98.025 | 1.00 | 29.59 |
| 1156 | CA | ASP | A | 366 | 37.895 | 61.028 | 99.465 | 1.00 | 31.14 |
| 1157 | CB | ASP | A | 366 | 39.394 | 60.887 | 99.824 | 1.00 | 29.81 |
| 1158 | CG | ASP | A | 366 | 40.289 | 61.840 | 99.143 | 1.00 | 31.81 |
| 1159 | OD1 | ASP | A | 366 | 39.942 | 63.003 | 98.946 | 1.00 | 34.95 |
| 1160 | OD2 | ASP | A | 366 | 41.394 | 61.408 | 98.737 | 1.00 | 39.23 |
| 1161 | C | ASP | A | 366 | 37.358 | 62.467 | 100.009 | 1.00 | 31.45 |
| 1162 | O | ASP | A | 366 | 36.950 | 63.298 | 99.266 | 1.00 | 27.08 |
| 1163 | N | ASP | A | 367 | 37.498 | 62.663 | 101.256 | 1.00 | 31.37 |
| 1164 | CA | ASP | A | 367 | 37.091 | 63.904 | 101.782 | 1.00 | 36.61 |
| 1165 | CB | ASP | A | 367 | 37.230 | 63.889 | 103.412 | 1.00 | 40.07 |
| 1166 | CG | ASP | A | 367 | 36.149 | 62.886 | 104.022 | 1.00 | 46.21 |
| 1167 | OD1 | ASP | A | 367 | 34.975 | 62.558 | 103.286 | 1.00 | 42.09 |
| 1168 | OD2 | ASP | A | 367 | 36.222 | 62.655 | 105.136 | 1.00 | 52.17 |
| 1169 | C | ASP | A | 367 | 37.681 | 65.205 | 101.248 | 1.00 | 35.74 |
| 1170 | O | ASP | A | 367 | 36.913 | 66.240 | 101.072 | 1.00 | 33.99 |
| 1171 | N | ALA | A | 368 | 38.844 | 65.150 | 100.887 | 1.00 | 29.92 |
| 1172 | CA | ALA | A | 368 | 39.457 | 66.373 | 100.299 | 1.00 | 30.49 |
| 1173 | CB | ALA | A | 368 | 41.098 | 66.112 | 100.092 | 1.00 | 26.50 |
| 1174 | C | ALA | A | 368 | 38.911 | 66.620 | 98.895 | 1.00 | 32.62 |
| 1175 | O | ALA | A | 368 | 38.829 | 67.801 | 98.523 | 1.00 | 36.37 |
| 1176 | N | GLU | A | 369 | 38.770 | 65.587 | 98.066 | 1.00 | 24.31 |
| 1177 | CA | GLU | A | 369 | 38.356 | 65.818 | 96.845 | 1.00 | 29.00 |
| 1178 | CB | GLU | A | 369 | 38.425 | 64.483 | 95.952 | 1.00 | 22.95 |
| 1179 | CG | GLU | A | 369 | 39.908 | 64.031 | 95.886 | 1.00 | 28.00 |
| 1180 | CD | GLU | A | 369 | 40.112 | 62.683 | 95.405 | 1.00 | 29.14 |
| 1181 | OE1 | GLU | A | 369 | 39.312 | 61.777 | 95.939 | 1.00 | 27.96 |
| 1182 | OE2 | GLU | A | 369 | 40.793 | 62.490 | 94.321 | 1.00 | 27.55 |
| 1183 | C | GLU | A | 369 | 36.897 | 66.411 | 96.735 | 1.00 | 28.40 |
| 1184 | O | GLU | A | 369 | 36.669 | 67.335 | 95.974 | 1.00 | 27.58 |
| 1185 | N | TYR | A | 370 | 36.018 | 65.878 | 97.623 | 1.00 | 27.68 |
| 1186 | CA | TYR | A | 370 | 34.700 | 66.410 | 97.861 | 1.00 | 25.95 |
| 1187 | CB | TYR | A | 370 | 33.971 | 65.677 | 98.870 | 1.00 | 27.29 |
| 1188 | CG | TYR | A | 370 | 33.067 | 64.500 | 98.317 | 1.00 | 30.67 |
| 1189 | CD1 | TYR | A | 370 | 33.375 | 63.179 | 98.520 | 1.00 | 29.65 |
| 1190 | CE1 | TYR | A | 370 | 32.559 | 62.169 | 98.044 | 1.00 | 24.96 |
| 1191 | CZ | TYR | A | 370 | 31.358 | 62.562 | 97.199 | 1.00 | 31.04 |
| 1192 | OH | TYR | A | 370 | 30.639 | 61.580 | 96.641 | 1.00 | 25.30 |
| 1193 | CE2 | TYR | A | 370 | 31.100 | 63.796 | 96.963 | 1.00 | 22.48 |
| 1194 | CD2 | TYR | A | 370 | 31.962 | 64.824 | 97.561 | 1.00 | 31.26 |
| 1195 | C | TYR | A | 370 | 34.743 | 67.837 | 98.315 | 1.00 | 28.81 |
| 1196 | O | TYR | A | 370 | 34.090 | 68.730 | 97.663 | 1.00 | 30.56 |
| 1197 | N | ALA | A | 371 | 35.610 | 68.180 | 99.251 | 1.00 | 27.51 |
| 1198 | CA | ALA | A | 371 | 35.671 | 69.536 | 99.783 | 1.00 | 22.07 |
| 1199 | CB | ALA | A | 371 | 36.645 | 69.600 | 101.022 | 1.00 | 26.40 |
| 1200 | C | ALA | A | 371 | 36.273 | 70.472 | 98.666 | 1.00 | 26.62 |
| 1201 | O | ALA | A | 371 | 35.793 | 71.530 | 98.503 | 1.00 | 27.10 |
| 1202 | N | LEU | A | 372 | 37.245 | 70.024 | 97.843 | 1.00 | 21.94 |
| 1203 | CA | LEU | A | 372 | 37.671 | 70.868 | 96.843 | 1.00 | 22.33 |
| 1204 | CB | LEU | A | 372 | 38.927 | 70.434 | 96.173 | 1.00 | 24.56 |
| 1205 | CG | LEU | A | 372 | 40.214 | 70.640 | 97.005 | 1.00 | 33.14 |
| 1206 | CD1 | LEU | A | 372 | 41.364 | 69.613 | 96.489 | 1.00 | 25.37 |
| 1207 | CD2 | LEU | A | 372 | 40.639 | 72.052 | 96.915 | 1.00 | 28.88 |
| 1208 | C | LEU | A | 372 | 36.621 | 71.088 | 95.683 | 1.00 | 22.60 |
| 1209 | O | LEU | A | 372 | 36.419 | 72.148 | 95.215 | 1.00 | 21.21 |
| 1210 | N | LEU | A | 373 | 35.964 | 69.974 | 95.242 | 1.00 | 25.15 |
| 1211 | CA | LEU | A | 373 | 34.993 | 70.043 | 94.214 | 1.00 | 22.32 |
| 1212 | CB | LEU | A | 373 | 34.404 | 68.597 | 94.110 | 1.00 | 21.91 |
| 1213 | CG | LEU | A | 373 | 33.494 | 68.425 | 92.811 | 1.00 | 25.88 |
| 1214 | CD1 | LEU | A | 373 | 34.350 | 68.939 | 91.548 | 1.00 | 22.43 |
| 1215 | CD2 | LEU | A | 373 | 33.089 | 66.945 | 92.660 | 1.00 | 18.83 |
| 1216 | C | LEU | A | 373 | 33.799 | 70.997 | 94.727 | 1.00 | 22.27 |
| 1217 | O | LEU | A | 373 | 33.235 | 71.741 | 93.878 | 1.00 | 22.92 |
| 1218 | N | ILE | A | 374 | 33.486 | 70.947 | 95.954 | 1.00 | 23.24 |
| 1219 | CA | ILE | A | 374 | 32.401 | 72.000 | 96.536 | 1.00 | 26.23 |
| 1220 | CB | ILE | A | 374 | 32.027 | 71.571 | 97.978 | 1.00 | 26.40 |
| 1221 | CG1 | ILE | A | 374 | 31.146 | 70.312 | 97.822 | 1.00 | 19.31 |
| 1222 | CD1 | ILE | A | 374 | 31.024 | 69.456 | 99.267 | 1.00 | 21.34 |
| 1223 | CG2 | ILE | A | 374 | 31.340 | 72.723 | 98.789 | 1.00 | 27.79 |
| 1224 | C | ILE | A | 374 | 32.924 | 73.408 | 96.489 | 1.00 | 25.86 |
| 1225 | O | ILE | A | 374 | 32.181 | 74.311 | 96.098 | 1.00 | 27.87 |
| 1226 | N | ALA | A | 375 | 34.227 | 73.650 | 96.815 | 1.00 | 24.08 |
| 1227 | CA | ALA | A | 375 | 34.790 | 75.051 | 96.714 | 1.00 | 25.33 |
| 1228 | CB | ALA | A | 375 | 36.278 | 75.151 | 97.234 | 1.00 | 31.90 |
| 1229 | C | ALA | A | 375 | 34.794 | 75.528 | 95.206 | 1.00 | 23.98 |
| 1230 | O | ALA | A | 375 | 34.462 | 76.633 | 94.933 | 1.00 | 26.40 |
| 1231 | N | ILE | A | 376 | 35.055 | 74.599 | 94.329 | 1.00 | 27.12 |
| 1232 | CA | ILE | A | 376 | 35.034 | 74.829 | 92.899 | 1.00 | 25.21 |
| 1233 | CB | ILE | A | 376 | 35.526 | 73.697 | 92.149 | 1.00 | 19.17 |
| 1234 | CG1 | ILE | A | 376 | 37.047 | 73.693 | 92.273 | 1.00 | 28.94 |
| 1235 | CD1 | ILE | A | 376 | 37.715 | 72.219 | 91.735 | 1.00 | 18.49 |
| 1236 | CG2 | ILE | A | 376 | 35.124 | 73.887 | 90.612 | 1.00 | 21.17 |
| 1237 | C | ILE | A | 376 | 33.591 | 75.176 | 92.431 | 1.00 | 26.62 |
| 1238 | O | ILE | A | 376 | 33.341 | 76.138 | 91.676 | 1.00 | 25.78 |
| 1239 | N | ASN | A | 377 | 32.665 | 74.415 | 92.956 | 1.00 | 26.58 |
| 1240 | CA | ASN | A | 377 | 31.236 | 74.589 | 92.619 | 1.00 | 27.26 |
| 1241 | CB | ASN | A | 377 | 30.414 | 73.465 | 93.392 | 1.00 | 32.12 |
| 1242 | CG | ASN | A | 377 | 28.883 | 73.492 | 93.003 | 1.00 | 33.33 |
| 1243 | OD1 | ASN | A | 377 | 28.115 | 74.333 | 93.591 | 1.00 | 34.73 |
| 1244 | ND2 | ASN | A | 377 | 28.480 | 72.681 | 92.131 | 1.00 | 31.41 |
| 1245 | C | ASN | A | 377 | 30.749 | 75.997 | 93.101 | 1.00 | 26.39 |
| 1246 | O | ASN | A | 377 | 30.098 | 76.708 | 92.346 | 1.00 | 24.60 |
| 1247 | N | ILE | A | 378 | 31.087 | 76.406 | 94.348 | 1.00 | 25.57 |
| 1248 | CA | ILE | A | 378 | 30.656 | 77.638 | 94.826 | 1.00 | 24.33 |
| 1249 | CB | ILE | A | 378 | 31.257 | 77.879 | 96.311 | 1.00 | 30.38 |
| 1250 | CG1 | ILE | A | 378 | 30.527 | 76.965 | 97.227 | 1.00 | 30.50 |
| 1251 | CD1 | ILE | A | 378 | 31.339 | 76.651 | 98.576 | 1.00 | 18.81 |
| 1252 | CG2 | ILE | A | 378 | 31.030 | 79.308 | 96.813 | 1.00 | 19.50 |
| 1253 | C | ILE | A | 378 | 31.144 | 78.792 | 93.969 | 1.00 | 25.80 |
| 1254 | O | ILE | A | 378 | 30.444 | 79.712 | 93.676 | 1.00 | 27.73 |
| 1255 | N | PHE | A | 379 | 32.372 | 78.795 | 93.503 | 1.00 | 27.64 |
| 1256 | CA | PHE | A | 379 | 32.922 | 79.862 | 92.687 | 1.00 | 24.14 |
| 1257 | CB | PHE | A | 379 | 34.452 | 80.154 | 93.022 | 1.00 | 22.61 |
| 1258 | CG | PHE | A | 379 | 34.733 | 80.516 | 94.437 | 1.00 | 24.59 |
| 1259 | CD1 | PHE | A | 379 | 34.273 | 81.719 | 94.913 | 1.00 | 27.74 |
| 1260 | CE1 | PHE | A | 379 | 34.674 | 82.266 | 96.127 | 1.00 | 33.61 |
| 1261 | CZ | PHE | A | 379 | 35.416 | 81.502 | 97.013 | 1.00 | 29.32 |
| 1262 | CE2 | PHE | A | 379 | 35.768 | 80.196 | 96.544 | 1.00 | 30.76 |
| 1263 | CD2 | PHE | A | 379 | 35.392 | 79.736 | 95.228 | 1.00 | 24.69 |
| 1264 | C | PHE | A | 379 | 32.696 | 79.763 | 91.182 | 1.00 | 30.53 |
| 1265 | O | PHE | A | 379 | 33.645 | 79.821 | 90.410 | 1.00 | 29.13 |
| 1266 | N | SER | A | 380 | 31.440 | 79.546 | 90.773 | 1.00 | 30.11 |
| 1267 | CA | SER | A | 380 | 31.049 | 79.544 | 89.382 | 1.00 | 28.70 |
| 1268 | CB | SER | A | 380 | 29.817 | 78.524 | 89.220 | 1.00 | 26.20 |
| 1269 | OG | SER | A | 380 | 30.267 | 77.214 | 89.815 | 1.00 | 25.22 |
| 1270 | C | SER | A | 380 | 30.583 | 80.844 | 88.829 | 1.00 | 25.94 |
| 1271 | O | SER | A | 380 | 29.671 | 81.382 | 89.306 | 1.00 | 27.86 |
| 1272 | N | ALA | A | 381 | 31.276 | 81.388 | 87.882 | 1.00 | 27.25 |
| 1273 | CA | ALA | A | 381 | 31.065 | 82.762 | 87.437 | 1.00 | 30.70 |
| 1274 | CB | ALA | A | 381 | 32.327 | 83.282 | 86.583 | 1.00 | 21.62 |
| 1275 | C | ALA | A | 381 | 29.755 | 82.911 | 86.622 | 1.00 | 32.31 |
| 1276 | O | ALA | A | 381 | 29.283 | 84.100 | 86.469 | 1.00 | 32.72 |
| 1277 | N | ASP | A | 382 | 29.169 | 81.863 | 86.171 | 1.00 | 29.52 |
| 1278 | CA | ASP | A | 382 | 27.968 | 81.981 | 85.348 | 1.00 | 30.16 |
| 1279 | CB | ASP | A | 382 | 28.024 | 80.802 | 84.249 | 1.00 | 33.90 |
| 1280 | CG | ASP | A | 382 | 27.902 | 79.417 | 84.934 | 1.00 | 41.16 |
| 1281 | OD1 | ASP | A | 382 | 28.184 | 79.312 | 86.135 | 1.00 | 40.14 |
| 1282 | OD2 | ASP | A | 382 | 27.449 | 78.515 | 84.394 | 1.00 | 50.33 |
| 1283 | C | ASP | A | 382 | 26.714 | 81.896 | 86.071 | 1.00 | 29.28 |
| 1284 | O | ASP | A | 382 | 25.666 | 81.833 | 85.479 | 1.00 | 30.47 |
| 1285 | N | ARG | A | 383 | 26.719 | 81.890 | 87.432 | 1.00 | 30.38 |
| 1286 | CA | ARG | A | 383 | 25.421 | 81.865 | 88.150 | 1.00 | 29.60 |
| 1287 | CB | ARG | A | 383 | 25.524 | 81.737 | 89.590 | 1.00 | 26.12 |
| 1288 | CG | ARG | A | 383 | 26.503 | 80.600 | 90.063 | 1.00 | 28.61 |
| 1289 | CD | ARG | A | 383 | 25.857 | 79.207 | 89.766 | 1.00 | 26.30 |
| 1290 | NE | ARG | A | 383 | 26.510 | 78.102 | 90.525 | 1.00 | 31.33 |
| 1291 | CZ | ARG | A | 383 | 26.154 | 76.823 | 90.516 | 1.00 | 32.37 |
| 1292 | NH1 | ARG | A | 383 | 25.111 | 76.412 | 89.784 | 1.00 | 26.78 |
| 1293 | NH2 | ARG | A | 383 | 26.843 | 76.032 | 91.264 | 1.00 | 30.05 |
| 1294 | C | ARG | A | 383 | 24.545 | 83.243 | 87.784 | 1.00 | 33.32 |
| 1295 | O | ARG | A | 383 | 25.139 | 84.301 | 87.551 | 1.00 | 38.00 |
| 1296 | N | PRO | A | 384 | 23.294 | 83.186 | 87.907 | 1.00 | 30.73 |
| 1297 | CA | PRO | A | 384 | 22.488 | 84.282 | 87.761 | 1.00 | 30.65 |
| 1298 | CB | PRO | A | 384 | 21.029 | 83.750 | 88.222 | 1.00 | 33.93 |
| 1299 | CG | PRO | A | 384 | 21.138 | 82.298 | 87.555 | 1.00 | 30.48 |
| 1300 | CD | PRO | A | 384 | 22.567 | 81.850 | 88.240 | 1.00 | 32.19 |
| 1301 | C | PRO | A | 384 | 22.735 | 85.434 | 88.723 | 1.00 | 28.49 |
| 1302 | O | PRO | A | 384 | 23.039 | 85.198 | 89.860 | 1.00 | 30.52 |
| 1303 | N | ASN | A | 385 | 22.779 | 86.627 | 88.247 | 1.00 | 33.92 |
| 1304 | CA | ASN | A | 385 | 22.909 | 87.850 | 88.864 | 1.00 | 28.88 |
| 1305 | CB | ASN | A | 385 | 21.950 | 87.815 | 89.956 | 1.00 | 39.11 |
| 1306 | CG | ASN | A | 385 | 20.556 | 87.667 | 89.404 | 1.00 | 45.66 |
| 1307 | OD1 | ASN | A | 385 | 19.776 | 86.886 | 90.011 | 1.00 | 44.67 |
| 1308 | ND2 | ASN | A | 385 | 20.231 | 88.327 | 88.320 | 1.00 | 37.49 |
| 1309 | C | ASN | A | 385 | 24.411 | 88.135 | 89.371 | 1.00 | 33.73 |
| 1310 | O | ASN | A | 385 | 24.598 | 89.183 | 90.049 | 1.00 | 36.83 |
| 1311 | N | VAL | A | 386 | 25.417 | 87.246 | 89.132 | 1.00 | 26.72 |
| 1312 | CA | VAL | A | 386 | 26.673 | 87.449 | 89.532 | 1.00 | 29.80 |
| 1313 | CB | VAL | A | 386 | 27.690 | 86.465 | 88.936 | 1.00 | 26.67 |
| 1314 | CG1 | VAL | A | 386 | 29.086 | 86.871 | 89.073 | 1.00 | 29.15 |
| 1315 | CG2 | VAL | A | 386 | 27.466 | 84.936 | 89.650 | 1.00 | 39.65 |
| 1316 | C | VAL | A | 386 | 27.173 | 88.968 | 89.082 | 1.00 | 29.38 |
| 1317 | O | VAL | A | 386 | 27.190 | 89.302 | 87.942 | 1.00 | 32.09 |
| 1318 | N | GLN | A | 387 | 27.603 | 89.705 | 90.002 | 1.00 | 28.15 |
| 1319 | CA | GLN | A | 387 | 28.024 | 91.166 | 89.544 | 1.00 | 30.91 |
| 1320 | CB | GLN | A | 387 | 27.797 | 92.077 | 90.694 | 1.00 | 31.43 |
| 1321 | CG | GLN | A | 387 | 26.434 | 92.007 | 91.257 | 1.00 | 27.96 |
| 1322 | CD | GLN | A | 387 | 26.370 | 93.075 | 92.471 | 1.00 | 34.77 |
| 1323 | OE1 | GLN | A | 387 | 25.322 | 93.682 | 92.727 | 1.00 | 32.52 |
| 1324 | NE2 | GLN | A | 387 | 27.419 | 93.109 | 93.204 | 1.00 | 27.17 |
| 1325 | C | GLN | A | 387 | 29.469 | 91.191 | 89.214 | 1.00 | 31.99 |
| 1326 | O | GLN | A | 387 | 29.869 | 91.925 | 88.354 | 1.00 | 31.63 |
| 1327 | N | GLU | A | 388 | 30.334 | 90.266 | 89.823 | 1.00 | 28.55 |
| 1328 | CA | GLU | A | 388 | 31.784 | 90.396 | 89.447 | 1.00 | 31.48 |
| 1329 | CB | GLU | A | 388 | 32.600 | 90.761 | 90.704 | 1.00 | 33.38 |
| 1330 | CG | GLU | A | 388 | 32.164 | 92.086 | 91.443 | 1.00 | 32.95 |
| 1331 | CD | GLU | A | 388 | 33.246 | 92.400 | 92.462 | 1.00 | 45.13 |
| 1332 | OE1 | GLU | A | 388 | 33.032 | 92.341 | 93.664 | 1.00 | 37.45 |
| 1333 | OE2 | GLU | A | 388 | 34.381 | 92.855 | 91.996 | 1.00 | 35.97 |
| 1334 | C | GLU | A | 388 | 32.227 | 88.922 | 88.985 | 1.00 | 31.66 |
| 1335 | O | GLU | A | 388 | 32.992 | 88.226 | 89.633 | 1.00 | 28.69 |
| 1336 | N | PRO | A | 389 | 31.764 | 88.550 | 87.802 | 1.00 | 31.67 |
| 1337 | CA | PRO | A | 389 | 32.119 | 87.193 | 87.288 | 1.00 | 33.14 |
| 1338 | CB | PRO | A | 389 | 31.345 | 87.202 | 85.757 | 1.00 | 31.36 |
| 1339 | CG | PRO | A | 389 | 31.401 | 88.824 | 85.428 | 1.00 | 30.48 |
| 1340 | CD | PRO | A | 389 | 31.011 | 89.459 | 86.763 | 1.00 | 27.05 |
| 1341 | C | PRO | A | 389 | 33.524 | 87.002 | 87.130 | 1.00 | 33.73 |
| 1342 | O | PRO | A | 389 | 33.989 | 85.937 | 87.387 | 1.00 | 34.71 |
| 1343 | N | GLY | A | 390 | 34.285 | 88.056 | 86.575 | 1.00 | 31.65 |
| 1344 | CA | GLY | A | 390 | 35.709 | 87.791 | 86.469 | 1.00 | 31.17 |
| 1345 | C | GLY | A | 390 | 36.469 | 87.587 | 87.823 | 1.00 | 31.16 |
| 1346 | O | GLY | A | 390 | 37.327 | 86.725 | 87.882 | 1.00 | 27.68 |
| 1347 | N | ARG | A | 391 | 35.986 | 88.273 | 88.926 | 1.00 | 30.04 |
| 1348 | CA | ARG | A | 391 | 36.571 | 87.993 | 90.215 | 1.00 | 30.27 |
| 1349 | CB | ARG | A | 391 | 36.059 | 88.919 | 91.287 | 1.00 | 31.81 |
| 1350 | CG | ARG | A | 391 | 36.876 | 88.961 | 92.606 | 1.00 | 36.05 |
| 1351 | CD | ARG | A | 391 | 38.301 | 89.717 | 92.397 | 1.00 | 41.83 |
| 1352 | NE | ARG | A | 391 | 39.252 | 88.791 | 91.670 | 1.00 | 44.01 |
| 1353 | CZ | ARG | A | 391 | 40.610 | 88.952 | 91.753 | 1.00 | 60.33 |
| 1354 | NH1 | ARG | A | 391 | 41.184 | 89.957 | 92.544 | 1.00 | 74.34 |
| 1355 | NH2 | ARG | A | 391 | 41.440 | 88.596 | 90.950 | 1.00 | 56.93 |
| 1356 | C | ARG | A | 391 | 36.244 | 86.563 | 90.653 | 1.00 | 29.49 |
| 1357 | O | ARG | A | 391 | 37.105 | 85.837 | 91.193 | 1.00 | 33.05 |
| 1358 | N | VAL | A | 392 | 34.992 | 86.194 | 90.444 | 1.00 | 29.58 |
| 1359 | CA | VAL | A | 392 | 34.548 | 84.837 | 90.857 | 1.00 | 28.07 |
| 1360 | CB | VAL | A | 392 | 33.045 | 84.512 | 90.527 | 1.00 | 25.28 |
| 1361 | CG1 | VAL | A | 392 | 32.675 | 82.920 | 90.768 | 1.00 | 20.82 |
| 1362 | CG2 | VAL | A | 392 | 32.179 | 85.402 | 91.408 | 1.00 | 18.99 |
| 1363 | C | VAL | A | 392 | 35.335 | 83.630 | 90.215 | 1.00 | 25.85 |
| 1364 | O | VAL | A | 392 | 35.683 | 82.672 | 90.839 | 1.00 | 24.37 |
| 1365 | N | GLU | A | 393 | 35.606 | 83.889 | 89.005 | 1.00 | 25.11 |
| 1366 | CA | GLU | A | 393 | 36.389 | 82.820 | 88.213 | 1.00 | 30.37 |
| 1367 | CB | GLU | A | 393 | 36.431 | 83.260 | 86.648 | 1.00 | 27.62 |
| 1368 | CG | GLU | A | 393 | 36.954 | 82.086 | 85.675 | 1.00 | 42.40 |
| 1369 | CD | GLU | A | 393 | 36.944 | 82.429 | 84.205 | 1.00 | 52.44 |
| 1370 | OE1 | GLU | A | 393 | 37.290 | 83.563 | 83.672 | 1.00 | 54.11 |
| 1371 | OE2 | GLU | A | 393 | 36.387 | 81.679 | 83.473 | 1.00 | 59.35 |
| 1372 | C | GLU | A | 393 | 37.862 | 82.831 | 88.653 | 1.00 | 24.86 |
| 1373 | O | GLU | A | 393 | 38.392 | 81.860 | 88.786 | 1.00 | 27.24 |
| 1374 | N | ALA | A | 394 | 38.392 | 83.974 | 89.054 | 1.00 | 28.71 |
| 1375 | CA | ALA | A | 394 | 39.853 | 83.981 | 89.532 | 1.00 | 27.69 |
| 1376 | CB | ALA | A | 394 | 40.413 | 85.489 | 89.467 | 1.00 | 26.95 |
| 1377 | C | ALA | A | 394 | 39.910 | 83.421 | 90.815 | 1.00 | 26.79 |
| 1378 | O | ALA | A | 394 | 40.871 | 82.728 | 91.065 | 1.00 | 37.81 |
| 1379 | N | LEU | A | 395 | 38.902 | 83.530 | 91.668 | 1.00 | 31.39 |
| 1380 | CA | LEU | A | 395 | 38.913 | 82.950 | 93.019 | 1.00 | 26.75 |
| 1381 | CB | LEU | A | 395 | 37.800 | 83.510 | 93.854 | 1.00 | 26.83 |
| 1382 | CG | LEU | A | 395 | 37.870 | 84.934 | 94.156 | 1.00 | 37.10 |
| 1383 | CD1 | LEU | A | 395 | 36.675 | 85.476 | 94.959 | 1.00 | 29.87 |
| 1384 | CD2 | LEU | A | 395 | 39.265 | 85.139 | 95.047 | 1.00 | 38.02 |
| 1385 | C | LEU | A | 395 | 38.691 | 81.426 | 92.890 | 1.00 | 29.53 |
| 1386 | O | LEU | A | 395 | 39.026 | 80.696 | 93.706 | 1.00 | 30.15 |
| 1387 | N | GLN | A | 396 | 38.087 | 80.955 | 91.788 | 1.00 | 30.18 |
| 1388 | CA | GLN | A | 396 | 37.872 | 79.548 | 91.564 | 1.00 | 30.65 |
| 1389 | CB | GLN | A | 396 | 36.798 | 79.306 | 90.484 | 1.00 | 26.71 |
| 1390 | CG | GLN | A | 396 | 36.379 | 77.817 | 90.332 | 1.00 | 27.41 |
| 1391 | CD | GLN | A | 396 | 35.582 | 77.586 | 89.046 | 1.00 | 30.01 |
| 1392 | OE1 | GLN | A | 396 | 36.112 | 77.887 | 87.939 | 1.00 | 34.46 |
| 1393 | NE2 | GLN | A | 396 | 34.449 | 77.071 | 89.157 | 1.00 | 23.49 |
| 1394 | C | GLN | A | 396 | 39.125 | 78.721 | 91.158 | 1.00 | 32.32 |
| 1395 | O | GLN | A | 396 | 39.346 | 77.612 | 91.588 | 1.00 | 31.21 |
| 1396 | N | GLN | A | 397 | 39.948 | 79.383 | 90.321 | 1.00 | 29.93 |
| 1397 | CA | GLN | A | 397 | 41.161 | 78.756 | 89.857 | 1.00 | 34.72 |
| 1398 | CB | GLN | A | 397 | 42.079 | 79.847 | 89.086 | 1.00 | 33.15 |
| 1399 | CG | GLN | A | 397 | 41.135 | 80.521 | 88.114 | 1.00 | 47.89 |
| 1400 | CD | GLN | A | 397 | 40.389 | 79.577 | 87.026 | 1.00 | 54.79 |
| 1401 | OE1 | GLN | A | 397 | 40.970 | 78.909 | 86.143 | 1.00 | 56.43 |
| 1402 | NE2 | GLN | A | 397 | 39.078 | 79.487 | 87.234 | 1.00 | 47.44 |
| 1403 | C | GLN | A | 397 | 42.079 | 78.034 | 90.789 | 1.00 | 31.50 |
| 1404 | O | GLN | A | 397 | 42.410 | 76.885 | 90.531 | 1.00 | 33.95 |
| 1405 | N | PRO | A | 398 | 42.488 | 78.652 | 91.901 | 1.00 | 32.17 |
| 1406 | CA | PRO | A | 398 | 43.417 | 77.939 | 92.836 | 1.00 | 33.30 |
| 1407 | CB | PRO | A | 398 | 43.694 | 78.885 | 94.123 | 1.00 | 30.34 |
| 1408 | CG | PRO | A | 398 | 43.243 | 80.298 | 93.380 | 1.00 | 33.80 |
| 1409 | CD | PRO | A | 398 | 42.194 | 80.090 | 92.358 | 1.00 | 27.53 |
| 1410 | C | PRO | A | 398 | 42.738 | 76.610 | 93.283 | 1.00 | 34.31 |
| 1411 | O | PRO | A | 398 | 43.492 | 75.605 | 93.531 | 1.00 | 37.90 |
| 1412 | N | TYR | A | 399 | 41.419 | 76.575 | 93.407 | 1.00 | 29.80 |
| 1413 | CA | TYR | A | 399 | 40.796 | 75.226 | 93.787 | 1.00 | 29.79 |
| 1414 | CB | TYR | A | 399 | 39.314 | 75.533 | 94.310 | 1.00 | 26.79 |
| 1415 | CG | TYR | A | 399 | 39.391 | 76.369 | 95.609 | 1.00 | 30.20 |
| 1416 | CD1 | TYR | A | 399 | 39.850 | 75.877 | 96.821 | 1.00 | 19.91 |
| 1417 | CE1 | TYR | A | 399 | 39.955 | 76.693 | 97.925 | 1.00 | 19.94 |
| 1418 | CZ | TYR | A | 399 | 39.474 | 78.001 | 97.852 | 1.00 | 28.75 |
| 1419 | OH | TYR | A | 399 | 39.610 | 78.833 | 98.939 | 1.00 | 32.92 |
| 1420 | CE2 | TYR | A | 399 | 39.080 | 78.598 | 96.645 | 1.00 | 27.99 |
| 1421 | CD2 | TYR | A | 399 | 39.036 | 77.786 | 95.541 | 1.00 | 32.38 |
| 1422 | C | TYR | A | 399 | 40.838 | 74.229 | 92.750 | 1.00 | 25.76 |
| 1423 | O | TYR | A | 399 | 40.948 | 73.086 | 92.974 | 1.00 | 27.61 |
| 1424 | N | VAL | A | 400 | 40.839 | 74.678 | 91.492 | 1.00 | 28.90 |
| 1425 | CA | VAL | A | 400 | 40.899 | 73.759 | 90.343 | 1.00 | 30.33 |
| 1426 | CB | VAL | A | 400 | 40.525 | 74.558 | 89.035 | 1.00 | 28.12 |
| 1427 | CG1 | VAL | A | 400 | 40.625 | 73.575 | 87.823 | 1.00 | 28.59 |
| 1428 | CG2 | VAL | A | 400 | 38.868 | 74.957 | 89.130 | 1.00 | 27.17 |
| 1429 | C | VAL | A | 400 | 42.343 | 73.282 | 90.248 | 1.00 | 32.13 |
| 1430 | O | VAL | A | 400 | 42.577 | 72.178 | 89.990 | 1.00 | 32.44 |
| 1431 | N | GLU | A | 401 | 43.294 | 74.187 | 90.477 | 1.00 | 34.53 |
| 1432 | CA | GLU | A | 401 | 44.728 | 73.837 | 90.461 | 1.00 | 31.84 |
| 1433 | CB | GLU | A | 401 | 45.553 | 75.137 | 90.815 | 1.00 | 35.52 |
| 1434 | CG | GLU | A | 401 | 45.554 | 76.108 | 89.776 | 1.00 | 50.30 |
| 1435 | CD | GLU | A | 401 | 46.461 | 77.373 | 90.237 | 1.00 | 71.99 |
| 1436 | OE1 | GLU | A | 401 | 47.623 | 76.993 | 90.690 | 1.00 | 75.14 |
| 1437 | OE2 | GLU | A | 401 | 46.044 | 78.745 | 90.202 | 1.00 | 60.74 |
| 1438 | C | GLU | A | 401 | 44.997 | 72.903 | 91.542 | 1.00 | 32.33 |
| 1439 | O | GLU | A | 401 | 45.615 | 71.865 | 91.249 | 1.00 | 33.73 |
| 1440 | N | ALA | A | 402 | 44.488 | 73.141 | 92.770 | 1.00 | 28.84 |
| 1441 | CA | ALA | A | 402 | 44.679 | 72.213 | 93.749 | 1.00 | 28.99 |
| 1442 | CB | ALA | A | 402 | 44.354 | 72.790 | 95.207 | 1.00 | 23.16 |
| 1443 | C | ALA | A | 402 | 44.135 | 70.909 | 93.551 | 1.00 | 33.91 |
| 1444 | O | ALA | A | 402 | 44.737 | 69.881 | 93.999 | 1.00 | 34.29 |
| 1445 | N | LEU | A | 403 | 42.856 | 70.792 | 93.023 | 1.00 | 31.11 |
| 1446 | CA | LEU | A | 403 | 42.286 | 69.471 | 92.813 | 1.00 | 29.68 |
| 1447 | CB | LEU | A | 403 | 40.732 | 69.679 | 92.331 | 1.00 | 31.27 |
| 1448 | CG | LEU | A | 403 | 39.888 | 68.502 | 92.054 | 1.00 | 22.85 |
| 1449 | CD1 | LEU | A | 403 | 39.975 | 67.378 | 93.212 | 1.00 | 12.67 |
| 1450 | CD2 | LEU | A | 403 | 38.459 | 68.653 | 91.865 | 1.00 | 30.67 |
| 1451 | C | LEU | A | 403 | 42.978 | 68.732 | 91.721 | 1.00 | 32.64 |
| 1452 | O | LEU | A | 403 | 43.081 | 67.511 | 91.743 | 1.00 | 29.24 |
| 1453 | N | LEU | A | 404 | 43.492 | 69.501 | 90.711 | 1.00 | 32.31 |
| 1454 | CA | LEU | A | 404 | 44.229 | 68.965 | 89.569 | 1.00 | 33.73 |
| 1455 | CB | LEU | A | 404 | 44.690 | 69.975 | 88.666 | 1.00 | 34.29 |
| 1456 | CG | LEU | A | 404 | 45.500 | 69.487 | 87.451 | 1.00 | 46.05 |
| 1457 | CD1 | LEU | A | 404 | 45.010 | 68.178 | 86.857 | 1.00 | 46.09 |
| 1458 | CD2 | LEU | A | 404 | 45.627 | 70.528 | 86.256 | 1.00 | 45.50 |
| 1459 | C | LEU | A | 404 | 45.476 | 68.218 | 90.066 | 1.00 | 41.28 |
| 1460 | O | LEU | A | 404 | 45.735 | 66.983 | 89.744 | 1.00 | 44.64 |
| 1461 | N | SER | A | 405 | 46.232 | 68.699 | 90.957 | 1.00 | 37.96 |
| 1462 | CA | SER | A | 405 | 47.467 | 68.220 | 91.476 | 1.00 | 44.39 |
| 1463 | CB | SER | A | 405 | 48.389 | 69.582 | 91.945 | 1.00 | 40.19 |
| 1464 | OG | SER | A | 405 | 48.190 | 69.736 | 93.256 | 1.00 | 52.87 |
| 1465 | C | SER | A | 405 | 47.136 | 67.132 | 92.536 | 1.00 | 43.99 |
| 1466 | O | SER | A | 405 | 47.820 | 66.053 | 92.550 | 1.00 | 42.67 |
| 1467 | N | TYR | A | 406 | 46.168 | 67.418 | 93.374 | 1.00 | 35.30 |
| 1468 | CA | TYR | A | 406 | 45.781 | 66.347 | 94.211 | 1.00 | 34.10 |
| 1469 | CB | TYR | A | 406 | 44.682 | 66.788 | 95.238 | 1.00 | 38.62 |
| 1470 | CG | TYR | A | 406 | 44.367 | 65.841 | 96.288 | 1.00 | 30.54 |
| 1471 | CD1 | TYR | A | 406 | 44.805 | 66.098 | 97.590 | 1.00 | 31.56 |
| 1472 | CE1 | TYR | A | 406 | 44.462 | 65.188 | 98.631 | 1.00 | 30.25 |
| 1473 | CZ | TYR | A | 406 | 43.804 | 64.086 | 98.329 | 1.00 | 26.94 |
| 1474 | OH | TYR | A | 406 | 43.423 | 63.233 | 99.436 | 1.00 | 40.42 |
| 1475 | CE2 | TYR | A | 406 | 43.301 | 63.860 | 97.184 | 1.00 | 30.74 |
| 1476 | CD2 | TYR | A | 406 | 43.573 | 64.795 | 96.035 | 1.00 | 26.35 |
| 1477 | C | TYR | A | 406 | 45.427 | 65.102 | 93.654 | 1.00 | 35.00 |
| 1478 | O | TYR | A | 406 | 45.805 | 63.964 | 94.172 | 1.00 | 38.25 |
| 1479 | N | THR | A | 407 | 44.775 | 65.109 | 92.611 | 1.00 | 36.43 |
| 1480 | CA | THR | A | 407 | 44.385 | 63.965 | 91.861 | 1.00 | 42.81 |
| 1481 | CB | THR | A | 407 | 43.137 | 64.087 | 90.910 | 1.00 | 32.55 |
| 1482 | OG1 | THR | A | 407 | 43.410 | 65.212 | 90.049 | 1.00 | 39.85 |
| 1483 | CG2 | THR | A | 407 | 41.899 | 64.584 | 91.677 | 1.00 | 40.39 |
| 1484 | C | THR | A | 407 | 45.514 | 63.245 | 91.065 | 1.00 | 42.98 |
| 1485 | O | THR | A | 407 | 45.504 | 62.114 | 90.801 | 1.00 | 42.83 |
| 1486 | N | ARG | A | 408 | 46.445 | 64.127 | 90.628 | 1.00 | 46.62 |
| 1487 | CA | ARG | A | 408 | 47.571 | 63.709 | 89.899 | 1.00 | 52.55 |
| 1488 | CB | ARG | A | 408 | 48.345 | 64.721 | 89.277 | 1.00 | 48.70 |
| 1489 | CG | ARG | A | 408 | 47.824 | 64.847 | 87.855 | 1.00 | 53.29 |
| 1490 | CD | ARG | A | 408 | 48.685 | 66.207 | 87.431 | 1.00 | 72.48 |
| 1491 | NE | ARG | A | 408 | 48.291 | 66.729 | 86.136 | 1.00 | 66.20 |
| 1492 | CZ | ARG | A | 408 | 48.839 | 67.847 | 85.647 | 1.00 | 64.96 |
| 1493 | NH1 | ARG | A | 408 | 49.702 | 68.464 | 86.414 | 1.00 | 63.99 |
| 1494 | NH2 | ARG | A | 408 | 48.478 | 68.288 | 84.370 | 1.00 | 69.82 |
| 1495 | C | ARG | A | 408 | 48.436 | 62.765 | 90.824 | 1.00 | 54.06 |
| 1496 | O | ARG | A | 408 | 48.991 | 61.713 | 90.391 | 1.00 | 49.02 |
| 1497 | N | ILE | A | 409 | 48.590 | 63.317 | 92.076 | 1.00 | 51.24 |
| 1498 | CA | ILE | A | 409 | 49.270 | 62.418 | 93.053 | 1.00 | 50.90 |
| 1499 | CB | ILE | A | 409 | 49.529 | 63.313 | 94.286 | 1.00 | 54.05 |
| 1500 | CG1 | ILE | A | 409 | 50.683 | 64.440 | 93.999 | 1.00 | 43.61 |
| 1501 | CD1 | ILE | A | 409 | 50.414 | 65.719 | 94.449 | 1.00 | 45.39 |
| 1502 | CG2 | ILE | A | 409 | 49.927 | 62.382 | 95.389 | 1.00 | 51.63 |
| 1503 | C | ILE | A | 409 | 48.661 | 61.134 | 93.530 | 1.00 | 54.26 |
| 1504 | O | ILE | A | 409 | 49.247 | 60.038 | 93.741 | 1.00 | 55.05 |
| 1505 | N | LYS | A | 410 | 47.343 | 61.152 | 93.544 | 1.00 | 54.70 |
| 1506 | CA | LYS | A | 410 | 46.555 | 60.110 | 94.103 | 1.00 | 50.99 |
| 1507 | CB | LYS | A | 410 | 45.161 | 60.665 | 94.544 | 1.00 | 47.69 |
| 1508 | CG | LYS | A | 410 | 44.229 | 59.623 | 94.947 | 1.00 | 51.67 |
| 1509 | CD | LYS | A | 410 | 42.947 | 60.151 | 95.775 | 1.00 | 55.94 |
| 1510 | CE | LYS | A | 410 | 41.975 | 59.090 | 96.148 | 1.00 | 44.96 |
| 1511 | NZ | LYS | A | 410 | 41.200 | 59.757 | 97.017 | 1.00 | 44.06 |
| 1512 | C | LYS | A | 410 | 46.509 | 59.074 | 93.032 | 1.00 | 49.55 |
| 1513 | O | LYS | A | 410 | 46.378 | 57.845 | 93.352 | 1.00 | 48.08 |
| 1514 | N | ARG | A | 411 | 46.407 | 59.465 | 91.775 | 1.00 | 52.32 |
| 1515 | CA | ARG | A | 411 | 46.319 | 58.384 | 90.703 | 1.00 | 52.88 |
| 1516 | CB | ARG | A | 411 | 44.877 | 58.019 | 90.242 | 1.00 | 54.89 |
| 1517 | CG | ARG | A | 411 | 44.056 | 57.302 | 91.277 | 1.00 | 67.48 |
| 1518 | CD | ARG | A | 411 | 44.746 | 56.064 | 91.754 | 1.00 | 66.12 |
| 1519 | NE | ARG | A | 411 | 44.451 | 55.896 | 93.193 | 1.00 | 68.99 |
| 1520 | CZ | ARG | A | 411 | 43.302 | 55.331 | 93.552 | 1.00 | 61.73 |
| 1521 | NH1 | ARG | A | 411 | 42.439 | 55.144 | 92.642 | 1.00 | 64.12 |
| 1522 | NH2 | ARG | A | 411 | 43.153 | 54.891 | 94.794 | 1.00 | 63.60 |
| 1523 | C | ARG | A | 411 | 47.153 | 58.796 | 89.515 | 1.00 | 57.53 |
| 1524 | O | ARG | A | 411 | 46.660 | 59.216 | 88.465 | 1.00 | 56.06 |
| 1525 | N | PRO | A | 412 | 48.481 | 58.714 | 89.670 | 1.00 | 60.42 |
| 1526 | CA | PRO | A | 412 | 49.366 | 59.100 | 88.559 | 1.00 | 60.45 |
| 1527 | CB | PRO | A | 412 | 50.792 | 58.654 | 89.055 | 1.00 | 56.65 |
| 1528 | CG | PRO | A | 412 | 50.638 | 58.752 | 90.601 | 1.00 | 69.55 |
| 1529 | CD | PRO | A | 412 | 49.227 | 58.268 | 90.861 | 1.00 | 65.25 |
| 1530 | C | PRO | A | 412 | 49.000 | 58.556 | 87.306 | 1.00 | 55.68 |
| 1531 | O | PRO | A | 412 | 49.182 | 59.133 | 86.255 | 1.00 | 54.06 |
| 1532 | N | GLN | A | 413 | 48.546 | 57.356 | 87.408 | 1.00 | 59.90 |
| 1533 | CA | GLN | A | 413 | 48.240 | 56.659 | 86.032 | 1.00 | 60.25 |
| 1534 | CB | GLN | A | 413 | 48.527 | 55.104 | 86.217 | 1.00 | 67.10 |
| 1535 | CG | GLN | A | 413 | 49.979 | 54.905 | 86.732 | 1.00 | 67.59 |
| 1536 | CD | GLN | A | 413 | 51.056 | 55.757 | 86.119 | 1.00 | 69.36 |
| 1537 | OE1 | GLN | A | 413 | 52.033 | 56.057 | 86.786 | 1.00 | 76.94 |
| 1538 | NE2 | GLN | A | 413 | 50.872 | 56.187 | 84.846 | 1.00 | 67.53 |
| 1539 | C | GLN | A | 413 | 47.009 | 56.779 | 85.481 | 1.00 | 59.10 |
| 1540 | O | GLN | A | 413 | 46.844 | 56.449 | 84.366 | 1.00 | 60.75 |
| 1541 | N | ASP | A | 414 | 45.896 | 57.364 | 86.336 | 1.00 | 63.03 |
| 1542 | CA | ASP | A | 414 | 44.555 | 57.575 | 85.738 | 1.00 | 55.65 |
| 1543 | CB | ASP | A | 414 | 43.565 | 56.927 | 86.446 | 1.00 | 53.04 |
| 1544 | CG | ASP | A | 414 | 42.148 | 57.197 | 85.879 | 1.00 | 59.05 |
| 1545 | OD1 | ASP | A | 414 | 41.969 | 58.000 | 84.911 | 1.00 | 68.82 |
| 1546 | OD2 | ASP | A | 414 | 41.126 | 56.572 | 86.289 | 1.00 | 57.97 |
| 1547 | C | ASP | A | 414 | 44.407 | 58.993 | 85.552 | 1.00 | 57.26 |
| 1548 | O | ASP | A | 414 | 43.743 | 59.555 | 86.419 | 1.00 | 60.96 |
| 1549 | N | GLN | A | 415 | 44.786 | 59.617 | 84.483 | 1.00 | 53.51 |
| 1550 | CA | GLN | A | 415 | 44.736 | 61.024 | 84.480 | 1.00 | 57.02 |
| 1551 | CB | GLN | A | 415 | 45.840 | 61.675 | 83.501 | 1.00 | 59.17 |
| 1552 | CG | GLN | A | 415 | 45.684 | 61.102 | 82.123 | 1.00 | 63.39 |
| 1553 | CD | GLN | A | 415 | 46.787 | 61.535 | 81.185 | 1.00 | 69.87 |
| 1554 | OE1 | GLN | A | 415 | 48.032 | 61.685 | 81.549 | 1.00 | 69.69 |
| 1555 | NE2 | GLN | A | 415 | 46.370 | 61.784 | 79.954 | 1.00 | 65.81 |
| 1556 | C | GLN | A | 415 | 43.300 | 61.480 | 83.949 | 1.00 | 59.76 |
| 1557 | O | GLN | A | 415 | 43.081 | 62.730 | 83.850 | 1.00 | 56.95 |
| 1558 | N | LEU | A | 416 | 42.429 | 60.561 | 83.629 | 1.00 | 50.69 |
| 1559 | CA | LEU | A | 416 | 41.262 | 60.915 | 83.182 | 1.00 | 51.95 |
| 1560 | CB | LEU | A | 416 | 40.604 | 59.909 | 82.263 | 1.00 | 53.16 |
| 1561 | CG | LEU | A | 416 | 40.848 | 60.187 | 80.807 | 1.00 | 57.50 |
| 1562 | CD1 | LEU | A | 416 | 39.912 | 59.258 | 80.067 | 1.00 | 61.02 |
| 1563 | CD2 | LEU | A | 416 | 40.336 | 61.419 | 80.230 | 1.00 | 61.68 |
| 1564 | C | LEU | A | 416 | 40.167 | 61.107 | 84.453 | 1.00 | 52.06 |
| 1565 | O | LEU | A | 416 | 39.004 | 61.326 | 84.201 | 1.00 | 48.57 |
| 1566 | N | ARG | A | 417 | 40.562 | 60.772 | 85.626 | 1.00 | 46.23 |
| 1567 | CA | ARG | A | 417 | 39.677 | 60.700 | 86.771 | 1.00 | 40.89 |
| 1568 | CB | ARG | A | 417 | 40.450 | 60.148 | 87.966 | 1.00 | 42.06 |
| 1569 | CG | ARG | A | 417 | 39.657 | 60.063 | 89.207 | 1.00 | 40.87 |
| 1570 | CD | ARG | A | 417 | 40.152 | 58.840 | 90.092 | 1.00 | 39.38 |
| 1571 | NE | ARG | A | 417 | 39.531 | 59.057 | 91.418 | 1.00 | 40.68 |
| 1572 | CZ | ARG | A | 417 | 39.876 | 59.801 | 92.391 | 1.00 | 35.79 |
| 1573 | NH1 | ARG | A | 417 | 40.779 | 60.686 | 92.220 | 1.00 | 36.07 |
| 1574 | NH2 | ARG | A | 417 | 39.229 | 59.755 | 93.478 | 1.00 | 41.09 |
| 1575 | C | ARG | A | 417 | 39.269 | 62.284 | 87.138 | 1.00 | 42.35 |
| 1576 | O | ARG | A | 417 | 38.155 | 62.476 | 87.367 | 1.00 | 42.23 |
| 1577 | N | PHE | A | 418 | 40.200 | 63.278 | 87.055 | 1.00 | 35.97 |
| 1578 | CA | PHE | A | 418 | 40.021 | 64.477 | 87.203 | 1.00 | 35.12 |
| 1579 | CB | PHE | A | 418 | 41.315 | 65.336 | 87.179 | 1.00 | 37.25 |
| 1580 | CG | PHE | A | 418 | 41.145 | 66.817 | 87.222 | 1.00 | 39.82 |
| 1581 | CD1 | PHE | A | 418 | 40.561 | 67.363 | 88.376 | 1.00 | 36.75 |
| 1582 | CE1 | PHE | A | 418 | 40.412 | 68.729 | 88.477 | 1.00 | 39.66 |
| 1583 | CZ | PHE | A | 418 | 40.913 | 69.635 | 87.493 | 1.00 | 38.39 |
| 1584 | CE2 | PHE | A | 418 | 41.303 | 69.057 | 86.338 | 1.00 | 41.04 |
| 1585 | CD2 | PHE | A | 418 | 41.411 | 67.677 | 86.155 | 1.00 | 38.75 |
| 1586 | C | PHE | A | 418 | 38.872 | 65.136 | 86.285 | 1.00 | 36.79 |
| 1587 | O | PHE | A | 418 | 37.975 | 65.773 | 86.756 | 1.00 | 34.40 |
| 1588 | N | PRO | A | 419 | 38.976 | 64.989 | 85.010 | 1.00 | 41.06 |
| 1589 | CA | PRO | A | 419 | 37.997 | 65.452 | 84.075 | 1.00 | 39.02 |
| 1590 | CB | PRO | A | 419 | 38.398 | 64.915 | 82.750 | 1.00 | 34.83 |
| 1591 | CG | PRO | A | 419 | 39.855 | 64.902 | 82.890 | 1.00 | 42.68 |
| 1592 | CD | PRO | A | 419 | 40.183 | 64.401 | 84.281 | 1.00 | 41.24 |
| 1593 | C | PRO | A | 419 | 36.585 | 64.848 | 84.467 | 1.00 | 39.16 |
| 1594 | O | PRO | A | 419 | 35.607 | 65.602 | 84.408 | 1.00 | 33.73 |
| 1595 | N | ARG | A | 420 | 36.600 | 63.577 | 84.771 | 1.00 | 35.41 |
| 1596 | CA | ARG | A | 420 | 35.250 | 62.979 | 85.162 | 1.00 | 37.98 |
| 1597 | CB | ARG | A | 420 | 35.532 | 61.478 | 85.670 | 1.00 | 42.52 |
| 1598 | CG | ARG | A | 420 | 35.774 | 60.547 | 84.395 | 1.00 | 46.42 |
| 1599 | CD | ARG | A | 420 | 36.028 | 59.128 | 84.767 | 1.00 | 50.85 |
| 1600 | NE | ARG | A | 420 | 37.172 | 58.711 | 83.941 | 1.00 | 64.11 |
| 1601 | CZ | ARG | A | 420 | 38.362 | 58.028 | 84.512 | 1.00 | 78.99 |
| 1602 | NH1 | ARG | A | 420 | 38.517 | 57.706 | 85.875 | 1.00 | 70.47 |
| 1603 | NH2 | ARG | A | 420 | 39.327 | 57.619 | 83.746 | 1.00 | 75.11 |
| 1604 | C | ARG | A | 420 | 34.767 | 63.697 | 86.391 | 1.00 | 36.80 |
| 1605 | O | ARG | A | 420 | 33.562 | 63.836 | 86.490 | 1.00 | 36.70 |
| 1606 | N | MET | A | 421 | 35.663 | 64.164 | 87.317 | 1.00 | 34.00 |
| 1607 | CA | MET | A | 421 | 35.064 | 64.925 | 88.565 | 1.00 | 36.38 |
| 1608 | CB | MET | A | 421 | 36.145 | 65.268 | 89.533 | 1.00 | 37.98 |
| 1609 | CG | MET | A | 421 | 37.009 | 64.221 | 89.994 | 1.00 | 42.30 |
| 1610 | SD | MET | A | 421 | 37.806 | 64.611 | 91.524 | 1.00 | 45.58 |
| 1611 | CE | MET | A | 421 | 38.300 | 62.962 | 92.178 | 1.00 | 44.14 |
| 1612 | C | MET | A | 421 | 34.438 | 66.104 | 88.237 | 1.00 | 35.14 |
| 1613 | O | MET | A | 421 | 33.286 | 66.389 | 88.607 | 1.00 | 30.64 |
| 1614 | N | LEU | A | 422 | 35.079 | 66.854 | 87.231 | 1.00 | 33.34 |
| 1615 | CA | LEU | A | 422 | 34.470 | 68.084 | 86.637 | 1.00 | 29.82 |
| 1616 | CB | LEU | A | 422 | 35.462 | 68.738 | 85.690 | 1.00 | 26.02 |
| 1617 | CG | LEU | A | 422 | 36.710 | 69.286 | 86.396 | 1.00 | 33.75 |
| 1618 | CD1 | LEU | A | 422 | 37.399 | 70.279 | 85.416 | 1.00 | 33.74 |
| 1619 | CD2 | LEU | A | 422 | 36.267 | 70.088 | 87.671 | 1.00 | 29.58 |
| 1620 | C | LEU | A | 422 | 33.294 | 67.769 | 85.929 | 1.00 | 27.96 |
| 1621 | O | LEU | A | 422 | 32.279 | 68.609 | 85.861 | 1.00 | 29.14 |
| 1622 | N | MET | A | 423 | 33.275 | 66.662 | 85.239 | 1.00 | 27.82 |
| 1623 | CA | MET | A | 423 | 32.065 | 66.404 | 84.346 | 1.00 | 32.40 |
| 1624 | CB | MET | A | 423 | 32.249 | 65.076 | 83.582 | 1.00 | 34.06 |
| 1625 | CG | MET | A | 423 | 31.903 | 65.119 | 82.269 | 1.00 | 49.31 |
| 1626 | SD | MET | A | 423 | 33.373 | 65.419 | 81.082 | 1.00 | 63.59 |
| 1627 | CE | MET | A | 423 | 34.237 | 63.848 | 81.579 | 1.00 | 57.13 |
| 1628 | C | MET | A | 423 | 30.806 | 66.227 | 85.315 | 1.00 | 30.73 |
| 1629 | O | MET | A | 423 | 29.727 | 66.509 | 84.918 | 1.00 | 29.61 |
| 1630 | N | LYS | A | 424 | 31.078 | 65.801 | 86.617 | 1.00 | 27.60 |
| 1631 | CA | LYS | A | 424 | 30.029 | 65.769 | 87.539 | 1.00 | 29.41 |
| 1632 | CB | LYS | A | 424 | 30.371 | 65.126 | 88.896 | 1.00 | 32.42 |
| 1633 | CG | LYS | A | 424 | 30.825 | 63.645 | 88.773 | 1.00 | 27.50 |
| 1634 | CD | LYS | A | 424 | 29.490 | 62.936 | 88.242 | 1.00 | 36.24 |
| 1635 | CE | LYS | A | 424 | 29.832 | 61.415 | 88.133 | 1.00 | 48.83 |
| 1636 | NZ | LYS | A | 424 | 29.139 | 60.782 | 86.961 | 1.00 | 49.91 |
| 1637 | C | LYS | A | 424 | 29.406 | 67.122 | 87.734 | 1.00 | 23.61 |
| 1638 | O | LYS | A | 424 | 28.142 | 67.230 | 88.073 | 1.00 | 30.83 |
| 1639 | N | LEU | A | 425 | 30.136 | 68.133 | 87.681 | 1.00 | 24.79 |
| 1640 | CA | LEU | A | 425 | 29.623 | 69.505 | 87.924 | 1.00 | 25.47 |
| 1641 | CB | LEU | A | 425 | 30.651 | 70.583 | 87.816 | 1.00 | 28.69 |
| 1642 | CG | LEU | A | 425 | 31.803 | 70.454 | 88.879 | 1.00 | 32.43 |
| 1643 | CD1 | LEU | A | 425 | 32.859 | 71.414 | 88.452 | 1.00 | 22.94 |
| 1644 | CD2 | LEU | A | 425 | 31.189 | 70.728 | 90.304 | 1.00 | 21.58 |
| 1645 | C | LEU | A | 425 | 28.500 | 69.750 | 86.792 | 1.00 | 29.35 |
| 1646 | O | LEU | A | 425 | 27.584 | 70.593 | 86.996 | 1.00 | 25.35 |
| 1647 | N | VAL | A | 426 | 28.621 | 69.038 | 85.684 | 1.00 | 27.10 |
| 1648 | CA | VAL | A | 426 | 27.675 | 69.220 | 84.602 | 1.00 | 28.71 |
| 1649 | CB | VAL | A | 426 | 28.187 | 68.537 | 83.182 | 1.00 | 29.02 |
| 1650 | CG1 | VAL | A | 426 | 27.146 | 68.755 | 82.165 | 1.00 | 25.81 |
| 1651 | CG2 | VAL | A | 426 | 29.488 | 69.113 | 82.785 | 1.00 | 22.35 |
| 1652 | C | VAL | A | 426 | 26.371 | 68.567 | 84.961 | 1.00 | 29.59 |
| 1653 | O | VAL | A | 426 | 25.330 | 69.197 | 84.784 | 1.00 | 25.85 |
| 1654 | N | SER | A | 427 | 26.486 | 67.295 | 85.446 | 1.00 | 25.25 |
| 1655 | CA | SER | A | 427 | 25.299 | 66.592 | 85.893 | 1.00 | 31.04 |
| 1656 | CB | SER | A | 427 | 25.646 | 65.239 | 86.541 | 1.00 | 29.34 |
| 1657 | OG | SER | A | 427 | 26.386 | 64.519 | 85.593 | 1.00 | 36.53 |
| 1658 | C | SER | A | 427 | 24.489 | 67.358 | 86.989 | 1.00 | 27.83 |
| 1659 | O | SER | A | 427 | 23.282 | 67.360 | 86.996 | 1.00 | 29.96 |
| 1660 | N | LEU | A | 428 | 25.241 | 68.094 | 87.855 | 1.00 | 25.96 |
| 1661 | CA | LEU | A | 428 | 24.574 | 68.851 | 88.874 | 1.00 | 25.88 |
| 1662 | CB | LEU | A | 428 | 25.615 | 69.374 | 89.908 | 1.00 | 26.13 |
| 1663 | CG | LEU | A | 428 | 26.242 | 68.388 | 90.765 | 1.00 | 28.00 |
| 1664 | CD1 | LEU | A | 428 | 27.333 | 68.878 | 91.619 | 1.00 | 25.90 |
| 1665 | CD2 | LEU | A | 428 | 25.160 | 67.651 | 91.617 | 1.00 | 25.47 |
| 1666 | C | LEU | A | 428 | 23.770 | 70.091 | 88.399 | 1.00 | 25.61 |
| 1667 | O | LEU | A | 428 | 22.797 | 70.426 | 89.070 | 1.00 | 27.26 |
| 1668 | N | ARG | A | 429 | 24.030 | 70.643 | 87.309 | 1.00 | 27.67 |
| 1669 | CA | ARG | A | 429 | 23.168 | 71.755 | 86.754 | 1.00 | 28.64 |
| 1670 | CB | ARG | A | 429 | 23.846 | 72.372 | 85.395 | 1.00 | 30.85 |
| 1671 | CG | ARG | A | 429 | 25.056 | 73.113 | 85.647 | 1.00 | 32.25 |
| 1672 | CD | ARG | A | 429 | 24.907 | 74.244 | 86.805 | 1.00 | 29.00 |
| 1673 | NE | ARG | A | 429 | 26.057 | 74.913 | 87.080 | 1.00 | 26.21 |
| 1674 | CZ | ARG | A | 429 | 26.474 | 76.062 | 86.553 | 1.00 | 34.64 |
| 1675 | NH1 | ARG | A | 429 | 25.821 | 76.717 | 85.593 | 1.00 | 22.70 |
| 1676 | NH2 | ARG | A | 429 | 27.717 | 76.569 | 86.962 | 1.00 | 23.50 |
| 1677 | C | ARG | A | 429 | 21.885 | 71.086 | 86.288 | 1.00 | 31.30 |
| 1678 | O | ARG | A | 429 | 20.758 | 71.548 | 86.717 | 1.00 | 32.96 |
| 1679 | N | THR | A | 430 | 21.963 | 69.916 | 85.636 | 1.00 | 30.33 |
| 1680 | CA | THR | A | 430 | 20.749 | 69.255 | 85.310 | 1.00 | 30.85 |
| 1681 | CB | THR | A | 430 | 20.935 | 68.120 | 84.531 | 1.00 | 35.73 |
| 1682 | OG1 | THR | A | 430 | 21.555 | 68.364 | 83.254 | 1.00 | 35.60 |
| 1683 | CG2 | THR | A | 430 | 19.717 | 67.347 | 84.442 | 1.00 | 37.10 |
| 1684 | C | THR | A | 430 | 19.923 | 68.792 | 86.599 | 1.00 | 37.17 |
| 1685 | O | THR | A | 430 | 18.697 | 68.925 | 86.688 | 1.00 | 34.48 |
| 1686 | N | LEU | A | 431 | 20.643 | 68.327 | 87.662 | 1.00 | 33.01 |
| 1687 | CA | LEU | A | 431 | 19.901 | 67.926 | 88.831 | 1.00 | 28.10 |
| 1688 | CB | LEU | A | 431 | 20.828 | 67.144 | 89.785 | 1.00 | 23.96 |
| 1689 | CG | LEU | A | 431 | 21.261 | 65.716 | 89.252 | 1.00 | 29.33 |
| 1690 | CD1 | LEU | A | 431 | 22.536 | 65.336 | 90.048 | 1.00 | 27.95 |
| 1691 | CD2 | LEU | A | 431 | 20.226 | 64.748 | 89.638 | 1.00 | 26.21 |
| 1692 | C | LEU | A | 431 | 19.241 | 69.061 | 89.469 | 1.00 | 30.75 |
| 1693 | O | LEU | A | 431 | 18.249 | 68.912 | 90.236 | 1.00 | 28.23 |
| 1694 | N | SER | A | 432 | 19.895 | 70.188 | 89.382 | 1.00 | 31.10 |
| 1695 | CA | SER | A | 432 | 19.306 | 71.382 | 90.063 | 1.00 | 34.73 |
| 1696 | CB | SER | A | 432 | 20.276 | 72.655 | 89.682 | 1.00 | 31.90 |
| 1697 | OG | SER | A | 432 | 19.792 | 73.610 | 90.459 | 1.00 | 34.94 |
| 1698 | C | SER | A | 432 | 17.914 | 71.747 | 89.269 | 1.00 | 33.59 |
| 1699 | O | SER | A | 432 | 16.960 | 72.011 | 89.974 | 1.00 | 32.44 |
| 1700 | N | SER | A | 433 | 17.903 | 71.747 | 87.942 | 1.00 | 27.95 |
| 1701 | CA | SER | A | 433 | 16.624 | 71.955 | 87.227 | 1.00 | 38.05 |
| 1702 | CB | SER | A | 433 | 16.663 | 71.739 | 85.662 | 1.00 | 32.77 |
| 1703 | OG | SER | A | 433 | 17.766 | 72.432 | 85.101 | 1.00 | 45.13 |
| 1704 | C | SER | A | 433 | 15.583 | 70.841 | 87.730 | 1.00 | 38.67 |
| 1705 | O | SER | A | 433 | 14.416 | 71.170 | 87.950 | 1.00 | 37.74 |
| 1706 | N | VAL | A | 434 | 16.048 | 69.555 | 87.805 | 1.00 | 36.17 |
| 1707 | CA | VAL | A | 434 | 15.102 | 68.479 | 88.235 | 1.00 | 32.23 |
| 1708 | CB | VAL | A | 434 | 15.767 | 67.297 | 88.257 | 1.00 | 32.68 |
| 1709 | CG1 | VAL | A | 434 | 14.919 | 66.195 | 88.855 | 1.00 | 30.28 |
| 1710 | CG2 | VAL | A | 434 | 16.210 | 66.792 | 86.800 | 1.00 | 31.93 |
| 1711 | C | VAL | A | 434 | 14.580 | 68.830 | 89.507 | 1.00 | 40.04 |
| 1712 | O | VAL | A | 434 | 13.439 | 68.531 | 89.830 | 1.00 | 38.10 |
| 1713 | N | HIS | A | 435 | 15.401 | 69.491 | 90.449 | 1.00 | 32.82 |
| 1714 | CA | HIS | A | 435 | 14.960 | 69.548 | 91.844 | 1.00 | 35.99 |
| 1715 | CB | HIS | A | 435 | 16.108 | 70.011 | 92.772 | 1.00 | 24.67 |
| 1716 | CG | HIS | A | 435 | 15.727 | 70.667 | 93.947 | 1.00 | 26.20 |
| 1717 | ND1 | HIS | A | 435 | 15.127 | 70.028 | 95.027 | 1.00 | 36.92 |
| 1718 | CE1 | HIS | A | 435 | 14.832 | 70.814 | 96.029 | 1.00 | 22.84 |
| 1719 | NE2 | HIS | A | 435 | 15.256 | 72.018 | 95.669 | 1.00 | 28.59 |
| 1720 | CD2 | HIS | A | 435 | 15.802 | 71.988 | 94.347 | 1.00 | 26.08 |
| 1721 | C | HIS | A | 435 | 14.007 | 70.990 | 91.768 | 1.00 | 37.15 |
| 1722 | O | HIS | A | 435 | 13.054 | 70.980 | 92.532 | 1.00 | 38.90 |
| 1723 | N | SER | A | 436 | 14.238 | 71.783 | 90.816 | 1.00 | 38.33 |
| 1724 | CA | SER | A | 436 | 13.399 | 73.019 | 90.653 | 1.00 | 44.55 |
| 1725 | CB | SER | A | 436 | 14.019 | 73.975 | 89.619 | 1.00 | 41.13 |
| 1726 | OG | SER | A | 436 | 15.049 | 74.750 | 90.099 | 1.00 | 54.11 |
| 1727 | C | SER | A | 436 | 12.003 | 72.383 | 90.259 | 1.00 | 42.03 |
| 1728 | O | SER | A | 436 | 10.981 | 72.945 | 90.755 | 1.00 | 42.41 |
| 1729 | N | GLU | A | 437 | 11.969 | 71.595 | 89.249 | 1.00 | 40.79 |
| 1730 | CA | GLU | A | 437 | 10.738 | 70.900 | 88.892 | 1.00 | 43.47 |
| 1731 | CB | GLU | A | 437 | 11.061 | 69.908 | 87.927 | 1.00 | 43.76 |
| 1732 | CG | GLU | A | 437 | 11.464 | 70.697 | 86.621 | 1.00 | 57.34 |
| 1733 | CD | GLU | A | 437 | 11.497 | 69.764 | 85.322 | 1.00 | 72.30 |
| 1734 | OE1 | GLU | A | 437 | 12.311 | 68.751 | 85.071 | 1.00 | 57.33 |
| 1735 | OE2 | GLU | A | 437 | 10.572 | 70.092 | 84.521 | 1.00 | 76.33 |
| 1736 | C | GLU | A | 437 | 9.970 | 70.284 | 90.017 | 1.00 | 46.04 |
| 1737 | O | GLU | A | 437 | 8.762 | 70.517 | 90.163 | 1.00 | 44.56 |
| 1738 | N | GLN | A | 438 | 10.698 | 69.499 | 90.812 | 1.00 | 46.25 |
| 1739 | CA | GLN | A | 438 | 10.148 | 68.959 | 91.933 | 1.00 | 43.05 |
| 1740 | CB | GLN | A | 438 | 11.160 | 67.968 | 92.704 | 1.00 | 45.87 |
| 1741 | CG | GLN | A | 438 | 10.821 | 67.763 | 94.135 | 1.00 | 49.29 |
| 1742 | CD | GLN | A | 438 | 9.663 | 66.598 | 94.156 | 1.00 | 55.63 |
| 1743 | OE1 | GLN | A | 438 | 9.841 | 65.568 | 93.633 | 1.00 | 55.13 |
| 1744 | NE2 | GLN | A | 438 | 8.524 | 67.028 | 94.581 | 1.00 | 51.65 |
| 1745 | C | GLN | A | 438 | 9.503 | 69.985 | 92.888 | 1.00 | 47.96 |
| 1746 | O | GLN | A | 438 | 8.328 | 69.879 | 93.358 | 1.00 | 51.78 |
| 1747 | N | VAL | A | 439 | 10.245 | 70.999 | 93.193 | 1.00 | 48.20 |
| 1748 | CA | VAL | A | 439 | 9.804 | 72.122 | 94.037 | 1.00 | 50.36 |
| 1749 | CB | VAL | A | 439 | 10.729 | 73.174 | 94.141 | 1.00 | 50.79 |
| 1750 | CG1 | VAL | A | 439 | 10.242 | 74.389 | 94.716 | 1.00 | 52.64 |
| 1751 | CG2 | VAL | A | 439 | 11.869 | 72.737 | 95.107 | 1.00 | 50.25 |
| 1752 | C | VAL | A | 439 | 8.379 | 72.797 | 93.386 | 1.00 | 57.07 |
| 1753 | O | VAL | A | 439 | 7.540 | 73.118 | 94.051 | 1.00 | 53.27 |
| 1754 | N | PHE | A | 440 | 8.308 | 72.784 | 92.083 | 1.00 | 57.76 |
| 1755 | CA | PHE | A | 440 | 7.226 | 73.397 | 91.444 | 1.00 | 66.63 |
| 1756 | CB | PHE | A | 440 | 7.548 | 73.880 | 89.971 | 1.00 | 62.94 |
| 1757 | CG | PHE | A | 440 | 6.717 | 73.131 | 88.956 | 1.00 | 73.05 |
| 1758 | CD1 | PHE | A | 440 | 6.802 | 71.677 | 88.717 | 1.00 | 85.94 |
| 1759 | CE1 | PHE | A | 440 | 5.902 | 70.839 | 87.909 | 1.00 | 73.25 |
| 1760 | CZ | PHE | A | 440 | 4.927 | 71.551 | 87.287 | 1.00 | 82.27 |
| 1761 | CE2 | PHE | A | 440 | 4.764 | 72.960 | 87.523 | 1.00 | 81.56 |
| 1762 | CD2 | PHE | A | 440 | 5.646 | 73.724 | 88.437 | 1.00 | 82.59 |
| 1763 | C | PHE | A | 440 | 6.060 | 72.275 | 91.573 | 1.00 | 66.87 |
| 1764 | O | PHE | A | 440 | 4.931 | 72.840 | 91.922 | 1.00 | 64.21 |
| 1765 | N | ALA | A | 441 | 6.261 | 71.010 | 91.246 | 1.00 | 60.46 |
| 1766 | CA | ALA | A | 441 | 5.173 | 70.271 | 91.239 | 1.00 | 65.34 |
| 1767 | CB | ALA | A | 441 | 5.647 | 68.767 | 90.689 | 1.00 | 61.82 |
| 1768 | C | ALA | A | 441 | 4.692 | 70.143 | 92.627 | 1.00 | 67.12 |
| 1769 | O | ALA | A | 441 | 3.901 | 69.380 | 92.845 | 1.00 | 73.23 |
| 1770 | N | LEU | A | 442 | 5.249 | 70.810 | 93.570 | 1.00 | 72.33 |
| 1771 | CA | LEU | A | 442 | 4.796 | 70.586 | 94.914 | 1.00 | 76.45 |
| 1772 | CB | LEU | A | 442 | 6.039 | 70.402 | 95.873 | 1.00 | 77.74 |
| 1773 | CG | LEU | A | 442 | 6.581 | 68.942 | 95.867 | 1.00 | 83.53 |
| 1774 | CD1 | LEU | A | 442 | 7.788 | 68.673 | 96.538 | 1.00 | 78.41 |
| 1775 | CD2 | LEU | A | 442 | 5.497 | 68.191 | 96.563 | 1.00 | 90.49 |
| 1776 | C | LEU | A | 442 | 4.015 | 71.796 | 95.331 | 1.00 | 78.34 |
| 1777 | O | LEU | A | 442 | 3.219 | 71.716 | 96.259 | 1.00 | 71.44 |
| 1778 | N | ARG | A | 443 | 4.350 | 72.960 | 94.684 | 1.00 | 78.55 |
| 1779 | CA | ARG | A | 443 | 3.805 | 74.263 | 95.098 | 1.00 | 78.60 |
| 1780 | CB | ARG | A | 443 | 4.504 | 75.444 | 94.406 | 1.00 | 77.87 |
| 1781 | CG | ARG | A | 443 | 3.994 | 76.809 | 94.630 | 1.00 | 85.09 |
| 1782 | CD | ARG | A | 443 | 4.855 | 77.895 | 94.376 | 1.00 | 86.30 |
| 1783 | NE | ARG | A | 443 | 4.485 | 78.950 | 95.329 | 1.00 | 85.73 |
| 1784 | CZ | ARG | A | 443 | 4.723 | 80.182 | 95.006 | 1.00 | 95.54 |
| 1785 | NH1 | ARG | A | 443 | 5.435 | 80.465 | 93.857 | 1.00 | 97.80 |
| 1786 | NH2 | ARG | A | 443 | 4.317 | 81.128 | 95.823 | 1.00 | 102.71 |
| 1787 | C | ARG | A | 443 | 2.264 | 74.178 | 94.653 | 1.00 | 84.02 |
| 1788 | O | ARG | A | 443 | 1.404 | 75.220 | 94.901 | 1.00 | 81.88 |
| 1789 | N | LEU | A | 444 | 1.934 | 72.989 | 94.066 | 1.00 | 84.66 |
| 1790 | CA | LEU | A | 444 | 0.650 | 72.774 | 93.482 | 1.00 | 86.44 |
| 1791 | CB | LEU | A | 444 | 0.724 | 72.607 | 91.957 | 1.00 | 84.71 |
| 1792 | CG | LEU | A | 444 | 1.212 | 73.978 | 91.396 | 1.00 | 90.51 |
| 1793 | CD1 | LEU | A | 444 | 1.943 | 73.586 | 90.075 | 1.00 | 93.16 |
| 1794 | CD2 | LEU | A | 444 | 0.213 | 75.394 | 91.338 | 1.00 | 107.20 |
| 1795 | C | LEU | A | 444 | 0.196 | 71.466 | 94.026 | 1.00 | 84.64 |
| 1796 | O | LEU | A | 444 | −0.162 | 70.680 | 93.258 | 1.00 | 86.26 |
| 1797 | N | ALA | A | 445 | 0.238 | 71.239 | 95.287 | 1.00 | 82.56 |
| 1798 | CA | ALA | A | 445 | −0.176 | 69.949 | 95.812 | 1.00 | 82.67 |
| 1799 | CB | ALA | A | 445 | 0.829 | 68.934 | 95.510 | 1.00 | 78.79 |
| 1800 | C | ALA | A | 445 | −0.464 | 70.183 | 97.356 | 1.00 | 85.71 |
| 1801 | O | ALA | A | 445 | −0.371 | 69.290 | 98.262 | 1.00 | 80.18 |
| 1802 | N | ALA | A | 446 | −0.832 | 71.475 | 97.600 | 1.00 | 92.16 |
| 1803 | CA | ALA | A | 446 | −1.182 | 72.091 | 98.988 | 1.00 | 97.18 |
| 1804 | CB | ALA | A | 446 | −2.513 | 71.589 | 99.617 | 1.00 | 99.10 |
| 1805 | C | ALA | A | 446 | 0.117 | 71.823 | 99.984 | 1.00 | 99.56 |
| 1806 | O | ALA | A | 446 | 0.058 | 71.252 | 101.258 | 1.00 | 100.00 |
| 1807 | N | LYS | A | 447 | 1.281 | 72.187 | 99.385 | 1.00 | 92.32 |
| 1808 | CA | LYS | A | 447 | 2.480 | 71.815 | 100.040 | 1.00 | 85.44 |
| 1809 | CB | LYS | A | 447 | 2.859 | 70.333 | 99.788 | 1.00 | 86.22 |
| 1810 | CG | LYS | A | 447 | 4.095 | 69.872 | 100.654 | 1.00 | 81.60 |
| 1811 | CD | LYS | A | 447 | 4.451 | 68.590 | 100.406 | 1.00 | 82.83 |
| 1812 | CE | LYS | A | 447 | 3.268 | 67.614 | 100.586 | 1.00 | 94.35 |
| 1813 | NZ | LYS | A | 447 | 3.579 | 66.004 | 100.401 | 1.00 | 95.29 |
| 1814 | C | LYS | A | 447 | 3.647 | 72.838 | 99.640 | 1.00 | 85.09 |
| 1815 | O | LYS | A | 447 | 4.149 | 72.846 | 98.461 | 1.00 | 87.81 |
| 1816 | N | LYS | A | 448 | 3.975 | 73.742 | 100.568 | 1.00 | 74.20 |
| 1817 | CA | LYS | A | 448 | 4.854 | 74.659 | 100.282 | 1.00 | 71.88 |
| 1818 | CB | LYS | A | 448 | 4.136 | 75.930 | 100.716 | 1.00 | 76.29 |
| 1819 | CG | LYS | A | 448 | 3.062 | 76.477 | 99.835 | 1.00 | 78.44 |
| 1820 | CD | LYS | A | 448 | 2.898 | 78.032 | 99.838 | 1.00 | 89.59 |
| 1821 | CE | LYS | A | 448 | 4.132 | 78.859 | 98.972 | 1.00 | 90.92 |
| 1822 | NZ | LYS | A | 448 | 4.726 | 80.124 | 99.676 | 1.00 | 78.09 |
| 1823 | C | LYS | A | 448 | 6.201 | 74.552 | 101.061 | 1.00 | 70.07 |
| 1824 | O | LYS | A | 448 | 6.299 | 74.181 | 102.250 | 1.00 | 68.77 |
| 1825 | N | LEU | A | 449 | 7.184 | 75.089 | 100.484 | 1.00 | 64.82 |
| 1826 | CA | LEU | A | 449 | 8.394 | 75.086 | 101.290 | 1.00 | 65.02 |
| 1827 | CB | LEU | A | 449 | 9.663 | 75.453 | 100.443 | 1.00 | 61.79 |
| 1828 | CG | LEU | A | 449 | 10.049 | 74.517 | 99.154 | 1.00 | 59.53 |
| 1829 | CD1 | LEU | A | 449 | 10.939 | 75.250 | 98.322 | 1.00 | 56.93 |
| 1830 | CD2 | LEU | A | 449 | 10.691 | 73.326 | 99.728 | 1.00 | 62.04 |
| 1831 | C | LEU | A | 449 | 8.353 | 75.993 | 102.569 | 1.00 | 61.28 |
| 1832 | O | LEU | A | 449 | 8.026 | 77.014 | 102.543 | 1.00 | 63.29 |
| 1833 | N | PRO | A | 450 | 8.901 | 75.553 | 103.645 | 1.00 | 63.84 |
| 1834 | CA | PRO | A | 450 | 8.980 | 76.446 | 104.765 | 1.00 | 58.74 |
| 1835 | CB | PRO | A | 450 | 9.770 | 75.580 | 105.907 | 1.00 | 64.22 |
| 1836 | CG | PRO | A | 450 | 10.837 | 74.448 | 104.960 | 1.00 | 57.82 |
| 1837 | CD | PRO | A | 450 | 9.913 | 74.288 | 103.764 | 1.00 | 60.43 |
| 1838 | C | PRO | A | 450 | 9.800 | 77.692 | 104.334 | 1.00 | 64.24 |
| 1839 | O | PRO | A | 450 | 10.649 | 77.870 | 103.396 | 1.00 | 67.98 |
| 1840 | N | PRO | A | 451 | 9.595 | 78.728 | 105.013 | 1.00 | 65.71 |
| 1841 | CA | PRO | A | 451 | 10.169 | 79.999 | 104.625 | 1.00 | 62.74 |
| 1842 | CB | PRO | A | 451 | 9.910 | 80.887 | 105.908 | 1.00 | 64.68 |
| 1843 | CG | PRO | A | 451 | 8.326 | 80.238 | 106.130 | 1.00 | 66.88 |
| 1844 | CD | PRO | A | 451 | 8.648 | 78.865 | 106.130 | 1.00 | 66.25 |
| 1845 | C | PRO | A | 451 | 11.679 | 80.077 | 104.296 | 1.00 | 58.54 |
| 1846 | O | PRO | A | 451 | 12.236 | 80.694 | 103.399 | 1.00 | 56.32 |
| 1847 | N | LEU | A | 452 | 12.416 | 79.445 | 105.043 | 1.00 | 58.56 |
| 1848 | CA | LEU | A | 452 | 13.817 | 79.391 | 104.748 | 1.00 | 55.44 |
| 1849 | CB | LEU | A | 452 | 14.568 | 78.564 | 105.805 | 1.00 | 61.54 |
| 1850 | CG | LEU | A | 452 | 14.894 | 79.450 | 107.096 | 1.00 | 60.59 |
| 1851 | CD1 | LEU | A | 452 | 15.037 | 78.629 | 108.494 | 1.00 | 60.37 |
| 1852 | CD2 | LEU | A | 452 | 16.218 | 80.025 | 106.721 | 1.00 | 55.27 |
| 1853 | C | LEU | A | 452 | 14.259 | 78.700 | 103.446 | 1.00 | 52.42 |
| 1854 | O | LEU | A | 452 | 14.980 | 79.281 | 102.671 | 1.00 | 51.12 |
| 1855 | N | LEU | A | 453 | 13.674 | 77.623 | 103.107 | 1.00 | 47.36 |
| 1856 | CA | LEU | A | 453 | 13.972 | 76.984 | 101.836 | 1.00 | 44.76 |
| 1857 | CB | LEU | A | 453 | 13.431 | 75.475 | 101.875 | 1.00 | 39.48 |
| 1858 | CG | LEU | A | 453 | 13.901 | 74.698 | 103.059 | 1.00 | 35.88 |
| 1859 | CD1 | LEU | A | 453 | 13.380 | 73.314 | 103.060 | 1.00 | 35.22 |
| 1860 | CD2 | LEU | A | 453 | 15.490 | 74.839 | 103.128 | 1.00 | 42.22 |
| 1861 | C | LEU | A | 453 | 13.376 | 77.699 | 100.831 | 1.00 | 46.42 |
| 1862 | O | LEU | A | 453 | 13.755 | 77.739 | 99.619 | 1.00 | 44.98 |
| 1863 | N | SER | A | 454 | 12.163 | 78.280 | 101.198 | 1.00 | 51.58 |
| 1864 | CA | SER | A | 454 | 11.330 | 79.070 | 100.141 | 1.00 | 52.68 |
| 1865 | CB | SER | A | 454 | 10.070 | 79.672 | 100.891 | 1.00 | 51.83 |
| 1866 | OG | SER | A | 454 | 9.261 | 80.092 | 99.852 | 1.00 | 53.35 |
| 1867 | C | SER | A | 454 | 12.180 | 80.297 | 99.505 | 1.00 | 45.72 |
| 1868 | O | SER | A | 454 | 12.132 | 80.501 | 98.407 | 1.00 | 44.83 |
| 1869 | N | GLU | A | 455 | 12.951 | 80.901 | 100.375 | 1.00 | 46.32 |
| 1870 | CA | GLU | A | 455 | 13.752 | 82.017 | 99.942 | 1.00 | 51.63 |
| 1871 | CB | GLU | A | 455 | 14.524 | 82.668 | 101.215 | 1.00 | 51.54 |
| 1872 | CG | GLU | A | 455 | 14.399 | 84.099 | 101.207 | 1.00 | 70.41 |
| 1873 | CD | GLU | A | 455 | 15.016 | 84.580 | 102.494 | 1.00 | 89.05 |
| 1874 | OE1 | GLU | A | 455 | 14.914 | 83.941 | 103.617 | 1.00 | 85.24 |
| 1875 | OE2 | GLU | A | 455 | 15.563 | 85.706 | 102.484 | 1.00 | 97.13 |
| 1876 | C | GLU | A | 455 | 14.934 | 81.578 | 98.853 | 1.00 | 58.03 |
| 1877 | O | GLU | A | 455 | 15.481 | 82.370 | 98.100 | 1.00 | 50.47 |
| 1878 | N | ILE | A | 456 | 15.328 | 80.264 | 98.862 | 1.00 | 54.73 |
| 1879 | CA | ILE | A | 456 | 16.244 | 79.882 | 97.829 | 1.00 | 49.19 |
| 1880 | CB | ILE | A | 456 | 17.125 | 78.697 | 98.531 | 1.00 | 49.97 |
| 1881 | CG1 | ILE | A | 456 | 17.408 | 79.156 | 99.957 | 1.00 | 51.26 |
| 1882 | CD1 | ILE | A | 456 | 18.058 | 77.967 | 100.909 | 1.00 | 50.93 |
| 1883 | CG2 | ILE | A | 456 | 18.346 | 78.644 | 97.712 | 1.00 | 47.70 |
| 1884 | C | ILE | A | 456 | 15.646 | 79.312 | 96.669 | 1.00 | 45.32 |
| 1885 | O | ILE | A | 456 | 16.109 | 79.467 | 95.617 | 1.00 | 45.86 |
| 1886 | N | TRP | A | 457 | 14.580 | 78.515 | 96.847 | 1.00 | 45.70 |
| 1887 | CA | TRP | A | 457 | 14.165 | 77.784 | 95.787 | 1.00 | 49.53 |
| 1888 | CB | TRP | A | 457 | 13.930 | 76.402 | 96.205 | 1.00 | 41.00 |
| 1889 | CG | TRP | A | 457 | 15.383 | 75.756 | 96.619 | 1.00 | 45.01 |
| 1890 | CD1 | TRP | A | 457 | 16.593 | 76.100 | 95.970 | 1.00 | 41.75 |
| 1891 | NE1 | TRP | A | 457 | 17.484 | 75.291 | 96.470 | 1.00 | 33.91 |
| 1892 | CE2 | TRP | A | 457 | 16.979 | 74.572 | 97.414 | 1.00 | 36.74 |
| 1893 | CD2 | TRP | A | 457 | 15.624 | 74.856 | 97.504 | 1.00 | 30.90 |
| 1894 | CE3 | TRP | A | 457 | 14.863 | 74.229 | 98.480 | 1.00 | 40.98 |
| 1895 | CZ3 | TRP | A | 457 | 15.407 | 73.254 | 99.341 | 1.00 | 40.95 |
| 1896 | CH2 | TRP | A | 457 | 16.852 | 72.924 | 99.238 | 1.00 | 35.65 |
| 1897 | CZ2 | TRP | A | 457 | 17.657 | 73.666 | 98.320 | 1.00 | 31.70 |
| 1898 | C | TRP | A | 457 | 12.761 | 78.288 | 95.009 | 1.00 | 55.80 |
| 1899 | O | TRP | A | 457 | 12.485 | 77.759 | 94.016 | 1.00 | 61.13 |
| 1900 | N | ASP | A | 458 | 12.044 | 79.154 | 95.538 | 1.00 | 59.64 |
| 1901 | CA | ASP | A | 458 | 10.846 | 79.680 | 94.653 | 1.00 | 67.90 |
| 1902 | CB | ASP | A | 458 | 9.572 | 79.760 | 95.500 | 1.00 | 69.43 |
| 1903 | CG | ASP | A | 458 | 8.757 | 78.391 | 95.413 | 1.00 | 62.47 |
| 1904 | OD1 | ASP | A | 458 | 8.323 | 77.972 | 94.165 | 1.00 | 59.31 |
| 1905 | OD2 | ASP | A | 458 | 8.678 | 77.795 | 96.429 | 1.00 | 51.87 |
| 1906 | C | ASP | A | 458 | 11.166 | 81.042 | 94.253 | 1.00 | 70.30 |
| 1907 | O | ASP | A | 458 | 10.615 | 81.528 | 93.355 | 1.00 | 69.09 |
| 1908 | N | VAL | A | 459 | 12.295 | 81.544 | 94.842 | 1.00 | 77.71 |
| 1909 | CA | VAL | A | 459 | 12.847 | 82.884 | 94.547 | 1.00 | 83.82 |
| 1910 | CB | VAL | A | 459 | 14.049 | 83.127 | 95.380 | 1.00 | 82.76 |
| 1911 | CG1 | VAL | A | 459 | 14.617 | 84.546 | 94.967 | 1.00 | 89.23 |
| 1912 | CG2 | VAL | A | 459 | 13.473 | 83.256 | 96.861 | 1.00 | 91.03 |
| 1913 | C | VAL | A | 459 | 13.168 | 83.060 | 93.128 | 1.00 | 84.97 |
| 1914 | O | VAL | A | 459 | 14.391 | 82.839 | 92.884 | 1.00 | 89.63 |
| 1915 | OXT | VAL | A | 459 | 12.329 | 83.419 | 92.298 | 1.00 | 86.95 |
| 1916 | N | GLU | B | 216 | 58.321 | 62.770 | 80.955 | 1.00 | 104.37 |
| 1917 | CA | GLU | B | 216 | 57.234 | 63.800 | 80.512 | 1.00 | 106.06 |
| 1918 | CB | GLU | B | 216 | 57.707 | 65.248 | 80.833 | 1.00 | 105.65 |
| 1919 | CG | GLU | B | 216 | 57.269 | 65.820 | 82.137 | 1.00 | 107.76 |
| 1920 | CD | GLU | B | 216 | 58.068 | 67.157 | 82.576 | 1.00 | 116.36 |
| 1921 | OE1 | GLU | B | 216 | 59.154 | 67.032 | 83.347 | 1.00 | 110.93 |
| 1922 | OE2 | GLU | B | 216 | 57.626 | 68.366 | 82.222 | 1.00 | 111.71 |
| 1923 | C | GLU | B | 216 | 57.034 | 63.737 | 78.964 | 1.00 | 106.02 |
| 1924 | O | GLU | B | 216 | 56.169 | 62.939 | 78.581 | 1.00 | 104.35 |
| 1925 | N | GLY | B | 217 | 57.779 | 64.617 | 78.135 | 1.00 | 107.22 |
| 1926 | CA | GLY | B | 217 | 57.789 | 64.688 | 76.647 | 1.00 | 105.67 |
| 1927 | C | GLY | B | 217 | 56.570 | 64.814 | 75.718 | 1.00 | 104.69 |
| 1928 | O | GLY | B | 217 | 56.440 | 65.771 | 74.937 | 1.00 | 106.74 |
| 1929 | N | VAL | B | 218 | 55.757 | 63.783 | 75.659 | 1.00 | 103.69 |
| 1930 | CA | VAL | B | 218 | 54.562 | 63.637 | 74.720 | 1.00 | 98.47 |
| 1931 | CB | VAL | B | 218 | 53.360 | 63.434 | 75.491 | 1.00 | 95.36 |
| 1932 | CG1 | VAL | B | 218 | 52.862 | 62.067 | 75.126 | 1.00 | 98.84 |
| 1933 | CG2 | VAL | B | 218 | 53.777 | 63.451 | 77.005 | 1.00 | 102.45 |
| 1934 | C | VAL | B | 218 | 54.102 | 64.529 | 73.625 | 1.00 | 94.37 |
| 1935 | O | VAL | B | 218 | 54.296 | 65.746 | 73.595 | 1.00 | 95.73 |
| 1936 | N | GLN | B | 219 | 53.393 | 63.853 | 72.775 | 1.00 | 89.99 |
| 1937 | CA | GLN | B | 219 | 52.771 | 64.333 | 71.624 | 1.00 | 87.50 |
| 1938 | CB | GLN | B | 219 | 53.740 | 64.376 | 70.514 | 1.00 | 88.79 |
| 1939 | CG | GLN | B | 219 | 54.370 | 65.737 | 70.260 | 1.00 | 99.78 |
| 1940 | CD | GLN | B | 219 | 55.356 | 65.774 | 68.921 | 1.00 | 102.13 |
| 1941 | OE1 | GLN | B | 219 | 56.064 | 66.782 | 68.670 | 1.00 | 99.81 |
| 1942 | NE2 | GLN | B | 219 | 55.316 | 64.709 | 68.100 | 1.00 | 97.21 |
| 1943 | C | GLN | B | 219 | 51.646 | 63.288 | 71.208 | 1.00 | 85.61 |
| 1944 | O | GLN | B | 219 | 51.756 | 62.153 | 71.651 | 1.00 | 87.59 |
| 1945 | N | LEU | B | 220 | 50.645 | 63.647 | 70.362 | 1.00 | 80.42 |
| 1946 | CA | LEU | B | 220 | 49.566 | 62.830 | 70.020 | 1.00 | 80.74 |
| 1947 | CB | LEU | B | 220 | 48.740 | 63.465 | 68.860 | 1.00 | 76.94 |
| 1948 | CG | LEU | B | 220 | 48.068 | 64.785 | 69.106 | 1.00 | 87.28 |
| 1949 | CD1 | LEU | B | 220 | 48.472 | 65.816 | 68.260 | 1.00 | 85.48 |
| 1950 | CD2 | LEU | B | 220 | 46.486 | 64.713 | 69.177 | 1.00 | 91.66 |
| 1951 | C | LEU | B | 220 | 50.039 | 61.426 | 69.453 | 1.00 | 86.14 |
| 1952 | O | LEU | B | 220 | 51.322 | 61.268 | 69.015 | 1.00 | 89.45 |
| 1953 | N | THR | B | 221 | 49.049 | 60.549 | 69.211 | 1.00 | 81.01 |
| 1954 | CA | THR | B | 221 | 49.299 | 59.278 | 68.777 | 1.00 | 80.01 |
| 1955 | CB | THR | B | 221 | 48.646 | 58.213 | 69.788 | 1.00 | 76.61 |
| 1956 | OG1 | THR | B | 221 | 47.242 | 58.289 | 69.709 | 1.00 | 77.41 |
| 1957 | CG2 | THR | B | 221 | 49.112 | 58.246 | 71.288 | 1.00 | 74.67 |
| 1958 | C | THR | B | 221 | 48.511 | 59.389 | 67.535 | 1.00 | 79.99 |
| 1959 | O | THR | B | 221 | 47.575 | 60.151 | 67.501 | 1.00 | 82.96 |
| 1960 | N | ALA | B | 222 | 48.738 | 58.531 | 66.521 | 1.00 | 76.73 |
| 1961 | CA | ALA | B | 222 | 48.058 | 58.652 | 65.304 | 1.00 | 71.98 |
| 1962 | CB | ALA | B | 222 | 48.579 | 57.685 | 64.258 | 1.00 | 83.74 |
| 1963 | C | ALA | B | 222 | 46.762 | 58.321 | 65.405 | 1.00 | 74.83 |
| 1964 | O | ALA | B | 222 | 45.833 | 58.670 | 64.494 | 1.00 | 69.80 |
| 1965 | N | ALA | B | 223 | 46.641 | 57.616 | 66.534 | 1.00 | 75.78 |
| 1966 | CA | ALA | B | 223 | 45.280 | 57.295 | 67.007 | 1.00 | 74.74 |
| 1967 | CB | ALA | B | 223 | 45.413 | 56.749 | 68.522 | 1.00 | 73.66 |
| 1968 | C | ALA | B | 223 | 44.429 | 58.548 | 66.964 | 1.00 | 66.81 |
| 1969 | O | ALA | B | 223 | 43.410 | 58.789 | 66.227 | 1.00 | 66.52 |
| 1970 | N | GLN | B | 224 | 44.952 | 59.362 | 67.758 | 1.00 | 67.53 |
| 1971 | CA | GLN | B | 224 | 44.188 | 60.715 | 67.901 | 1.00 | 69.66 |
| 1972 | CB | GLN | B | 224 | 44.506 | 61.233 | 69.174 | 1.00 | 63.64 |
| 1973 | CG | GLN | B | 224 | 45.919 | 61.023 | 69.742 | 1.00 | 54.82 |
| 1974 | CD | GLN | B | 224 | 45.914 | 60.881 | 71.222 | 1.00 | 61.31 |
| 1975 | OE1 | GLN | B | 224 | 46.978 | 61.214 | 71.967 | 1.00 | 60.03 |
| 1976 | NE2 | GLN | B | 224 | 44.709 | 60.497 | 71.807 | 1.00 | 60.04 |
| 1977 | C | GLN | B | 224 | 44.141 | 61.725 | 66.676 | 1.00 | 66.55 |
| 1978 | O | GLN | B | 224 | 42.993 | 62.319 | 66.151 | 1.00 | 62.07 |
| 1979 | N | GLU | B | 225 | 45.278 | 61.728 | 66.021 | 1.00 | 70.62 |
| 1980 | CA | GLU | B | 225 | 45.190 | 62.593 | 64.761 | 1.00 | 68.19 |
| 1981 | CB | GLU | B | 225 | 46.480 | 62.689 | 64.237 | 1.00 | 77.82 |
| 1982 | CG | GLU | B | 225 | 47.079 | 64.105 | 64.057 | 1.00 | 81.01 |
| 1983 | CD | GLU | B | 225 | 48.649 | 64.215 | 64.181 | 1.00 | 70.84 |
| 1984 | OE1 | GLU | B | 225 | 49.248 | 63.364 | 64.898 | 1.00 | 75.02 |
| 1985 | OE2 | GLU | B | 225 | 49.263 | 65.156 | 63.577 | 1.00 | 72.57 |
| 1986 | C | GLU | B | 225 | 44.372 | 62.290 | 63.734 | 1.00 | 62.50 |
| 1987 | O | GLU | B | 225 | 43.317 | 63.140 | 63.169 | 1.00 | 60.80 |
| 1988 | N | ALA | B | 226 | 44.342 | 60.972 | 63.597 | 1.00 | 69.45 |
| 1989 | CA | ALA | B | 226 | 42.963 | 60.527 | 62.746 | 1.00 | 70.76 |
| 1990 | CB | ALA | B | 226 | 42.721 | 59.008 | 62.661 | 1.00 | 77.42 |
| 1991 | C | ALA | B | 226 | 41.982 | 61.171 | 63.218 | 1.00 | 65.26 |
| 1992 | O | ALA | B | 226 | 41.034 | 61.841 | 62.464 | 1.00 | 65.23 |
| 1993 | N | MET | B | 227 | 41.615 | 60.849 | 64.432 | 1.00 | 73.79 |
| 1994 | CA | MET | B | 227 | 40.135 | 61.403 | 64.931 | 1.00 | 58.91 |
| 1995 | CB | MET | B | 227 | 40.047 | 60.908 | 66.356 | 1.00 | 63.81 |
| 1996 | CG | MET | B | 227 | 38.792 | 61.642 | 67.072 | 1.00 | 63.60 |
| 1997 | SD | MET | B | 227 | 39.312 | 61.820 | 68.847 | 1.00 | 74.22 |
| 1998 | CE | MET | B | 227 | 40.123 | 60.366 | 69.617 | 1.00 | 64.78 |
| 1999 | C | MET | B | 227 | 40.050 | 62.822 | 64.740 | 1.00 | 53.86 |
| 2000 | O | MET | B | 227 | 38.919 | 63.341 | 64.335 | 1.00 | 58.35 |
| 2001 | N | ILE | B | 228 | 41.123 | 63.528 | 64.912 | 1.00 | 52.26 |
| 2002 | CA | ILE | B | 228 | 40.924 | 64.993 | 64.635 | 1.00 | 54.73 |
| 2003 | CB | ILE | B | 228 | 42.273 | 65.657 | 65.304 | 1.00 | 57.09 |
| 2004 | CG1 | ILE | B | 228 | 42.424 | 65.342 | 66.623 | 1.00 | 54.21 |
| 2005 | CD1 | ILE | B | 228 | 43.637 | 65.693 | 67.065 | 1.00 | 71.68 |
| 2006 | CG2 | ILE | B | 228 | 42.290 | 67.084 | 65.280 | 1.00 | 66.30 |
| 2007 | C | ILE | B | 228 | 40.488 | 65.434 | 63.252 | 1.00 | 59.28 |
| 2008 | O | ILE | B | 228 | 39.399 | 66.221 | 63.112 | 1.00 | 55.72 |
| 2009 | N | GLN | B | 229 | 41.717 | 65.136 | 62.373 | 1.00 | 67.17 |
| 2010 | CA | GLN | B | 229 | 41.220 | 65.425 | 61.025 | 1.00 | 56.82 |
| 2011 | CB | GLN | B | 229 | 42.067 | 64.717 | 60.025 | 1.00 | 72.34 |
| 2012 | CG | GLN | B | 229 | 42.784 | 65.636 | 58.754 | 1.00 | 63.55 |
| 2013 | CD | GLN | B | 229 | 44.232 | 65.970 | 59.210 | 1.00 | 79.42 |
| 2014 | OE1 | GLN | B | 229 | 44.605 | 67.084 | 59.893 | 1.00 | 79.95 |
| 2015 | NE2 | GLN | B | 229 | 45.174 | 64.947 | 58.928 | 1.00 | 83.13 |
| 2016 | C | GLN | B | 229 | 39.932 | 65.027 | 60.659 | 1.00 | 53.09 |
| 2017 | O | GLN | B | 229 | 38.929 | 65.709 | 60.133 | 1.00 | 54.06 |
| 2018 | N | GLN | B | 230 | 39.742 | 63.883 | 60.942 | 1.00 | 57.65 |
| 2019 | CA | GLN | B | 230 | 38.128 | 63.382 | 60.670 | 1.00 | 62.36 |
| 2020 | CB | GLN | B | 230 | 37.940 | 61.858 | 61.286 | 1.00 | 68.75 |
| 2021 | CG | GLN | B | 230 | 36.517 | 61.303 | 61.428 | 1.00 | 66.09 |
| 2022 | CD | GLN | B | 230 | 36.302 | 59.984 | 60.740 | 1.00 | 72.78 |
| 2023 | OE1 | GLN | B | 230 | 35.581 | 59.153 | 61.300 | 1.00 | 79.24 |
| 2024 | NE2 | GLN | B | 230 | 36.751 | 59.851 | 59.385 | 1.00 | 79.99 |
| 2025 | C | GLN | B | 230 | 37.132 | 64.259 | 61.023 | 1.00 | 58.98 |
| 2026 | O | GLN | B | 230 | 36.234 | 64.761 | 60.243 | 1.00 | 65.60 |
| 2027 | N | LEU | B | 231 | 37.184 | 64.567 | 62.325 | 1.00 | 61.17 |
| 2028 | CA | LEU | B | 231 | 36.271 | 65.559 | 63.009 | 1.00 | 56.23 |
| 2029 | CB | LEU | B | 231 | 36.527 | 65.721 | 64.543 | 1.00 | 66.04 |
| 2030 | CG | LEU | B | 231 | 36.078 | 64.613 | 65.585 | 1.00 | 68.69 |
| 2031 | CD1 | LEU | B | 231 | 36.486 | 65.056 | 66.929 | 1.00 | 57.79 |
| 2032 | CD2 | LEU | B | 231 | 34.422 | 64.277 | 65.434 | 1.00 | 52.98 |
| 2033 | C | LEU | B | 231 | 36.329 | 66.995 | 62.437 | 1.00 | 54.78 |
| 2034 | O | LEU | B | 231 | 35.259 | 67.574 | 62.231 | 1.00 | 57.81 |
| 2035 | N | VAL | B | 232 | 37.321 | 67.640 | 62.034 | 1.00 | 62.54 |
| 2036 | CA | VAL | B | 232 | 37.149 | 69.043 | 61.447 | 1.00 | 66.58 |
| 2037 | CB | VAL | B | 232 | 38.485 | 69.812 | 61.477 | 1.00 | 67.20 |
| 2038 | CG1 | VAL | B | 232 | 39.817 | 69.150 | 62.503 | 1.00 | 61.81 |
| 2039 | CG2 | VAL | B | 232 | 38.915 | 69.807 | 60.241 | 1.00 | 79.63 |
| 2040 | C | VAL | B | 232 | 36.493 | 69.154 | 59.990 | 1.00 | 65.40 |
| 2041 | O | VAL | B | 232 | 35.559 | 69.981 | 59.698 | 1.00 | 60.73 |
| 2042 | N | ALA | B | 233 | 36.848 | 68.108 | 59.206 | 1.00 | 68.53 |
| 2043 | CA | ALA | B | 233 | 36.221 | 68.049 | 57.772 | 1.00 | 66.56 |
| 2044 | CB | ALA | B | 233 | 36.610 | 67.001 | 57.095 | 1.00 | 65.10 |
| 2045 | C | ALA | B | 233 | 34.713 | 68.055 | 57.800 | 1.00 | 73.51 |
| 2046 | O | ALA | B | 233 | 34.001 | 68.933 | 56.823 | 1.00 | 68.35 |
| 2047 | N | ALA | B | 234 | 34.180 | 67.208 | 58.815 | 1.00 | 67.41 |
| 2048 | CA | ALA | B | 234 | 32.789 | 67.089 | 58.936 | 1.00 | 68.39 |
| 2049 | CB | ALA | B | 234 | 32.406 | 66.087 | 60.009 | 1.00 | 68.31 |
| 2050 | C | ALA | B | 234 | 32.062 | 68.334 | 59.125 | 1.00 | 75.66 |
| 2051 | O | ALA | B | 234 | 30.789 | 68.666 | 58.669 | 1.00 | 79.35 |
| 2052 | N | GLN | B | 235 | 32.785 | 69.152 | 59.785 | 1.00 | 82.05 |
| 2053 | CA | GLN | B | 235 | 32.111 | 70.403 | 60.090 | 1.00 | 85.91 |
| 2054 | CB | GLN | B | 235 | 32.565 | 70.876 | 61.676 | 1.00 | 84.49 |
| 2055 | CG | GLN | B | 235 | 33.819 | 71.201 | 61.907 | 1.00 | 89.12 |
| 2056 | CD | GLN | B | 235 | 34.175 | 71.656 | 63.242 | 1.00 | 100.23 |
| 2057 | OE1 | GLN | B | 235 | 35.396 | 72.090 | 63.469 | 1.00 | 93.13 |
| 2058 | NE2 | GLN | B | 235 | 33.222 | 71.508 | 64.165 | 1.00 | 96.59 |
| 2059 | C | GLN | B | 235 | 32.178 | 71.421 | 59.033 | 1.00 | 83.43 |
| 2060 | O | GLN | B | 235 | 31.554 | 72.471 | 59.051 | 1.00 | 82.43 |
| 2061 | N | LEU | B | 236 | 33.072 | 71.063 | 58.122 | 1.00 | 91.29 |
| 2062 | CA | LEU | B | 236 | 33.234 | 71.793 | 56.844 | 1.00 | 89.72 |
| 2063 | CB | LEU | B | 236 | 34.558 | 71.576 | 56.363 | 1.00 | 87.89 |
| 2064 | CG | LEU | B | 236 | 35.548 | 72.011 | 57.426 | 1.00 | 95.20 |
| 2065 | CD1 | LEU | B | 236 | 36.945 | 72.282 | 56.958 | 1.00 | 87.66 |
| 2066 | CD2 | LEU | B | 236 | 35.131 | 73.303 | 58.312 | 1.00 | 108.16 |
| 2067 | C | LEU | B | 236 | 32.286 | 71.245 | 55.881 | 1.00 | 88.08 |
| 2068 | O | LEU | B | 236 | 31.926 | 71.960 | 55.003 | 1.00 | 93.63 |
| 2069 | N | GLN | B | 237 | 31.852 | 69.992 | 56.046 | 1.00 | 90.20 |
| 2070 | CA | GLN | B | 237 | 30.803 | 69.360 | 55.060 | 1.00 | 92.05 |
| 2071 | CB | GLN | B | 237 | 30.467 | 67.906 | 55.468 | 1.00 | 90.65 |
| 2072 | CG | GLN | B | 237 | 29.549 | 67.046 | 54.443 | 1.00 | 97.30 |
| 2073 | CD | GLN | B | 237 | 28.369 | 66.278 | 55.109 | 1.00 | 94.00 |
| 2074 | OE1 | GLN | B | 237 | 28.258 | 65.037 | 55.016 | 1.00 | 103.21 |
| 2075 | NE2 | GLN | B | 237 | 27.481 | 66.985 | 55.761 | 1.00 | 97.17 |
| 2076 | C | GLN | B | 237 | 29.587 | 70.312 | 55.242 | 1.00 | 90.77 |
| 2077 | O | GLN | B | 237 | 28.938 | 70.601 | 54.263 | 1.00 | 91.75 |
| 2078 | N | ALA | B | 238 | 29.372 | 70.672 | 56.527 | 1.00 | 86.00 |
| 2079 | CA | ALA | B | 238 | 28.534 | 71.693 | 57.027 | 1.00 | 90.09 |
| 2080 | CB | ALA | B | 238 | 29.561 | 72.807 | 57.626 | 1.00 | 88.02 |
| 2081 | C | ALA | B | 238 | 27.354 | 72.493 | 56.322 | 1.00 | 90.02 |
| 2082 | O | ALA | B | 238 | 26.284 | 72.317 | 56.754 | 1.00 | 90.59 |
| 2083 | N | ALA | B | 239 | 27.518 | 73.378 | 55.305 | 1.00 | 91.29 |
| 2084 | CA | ALA | B | 239 | 26.354 | 74.165 | 54.825 | 1.00 | 94.57 |
| 2085 | CB | ALA | B | 239 | 26.022 | 75.307 | 55.798 | 1.00 | 96.72 |
| 2086 | C | ALA | B | 239 | 26.437 | 74.827 | 53.438 | 1.00 | 96.93 |
| 2087 | O | ALA | B | 239 | 27.583 | 75.241 | 53.091 | 1.00 | 90.16 |
| 2088 | N | ALA | B | 240 | 25.177 | 75.029 | 52.800 | 1.00 | 96.92 |
| 2089 | CA | ALA | B | 240 | 24.941 | 75.529 | 51.426 | 1.00 | 98.11 |
| 2090 | CB | ALA | B | 240 | 23.284 | 75.630 | 51.054 | 1.00 | 99.06 |
| 2091 | C | ALA | B | 240 | 25.656 | 76.871 | 51.061 | 1.00 | 100.78 |
| 2092 | O | ALA | B | 240 | 25.725 | 77.284 | 49.807 | 1.00 | 100.97 |
| 2093 | N | ALA | B | 242 | 21.565 | 79.128 | 48.299 | 1.00 | 106.20 |
| 2094 | CA | ALA | B | 242 | 20.635 | 78.973 | 49.457 | 1.00 | 108.35 |
| 2095 | CB | ALA | B | 242 | 20.497 | 80.423 | 50.236 | 1.00 | 109.25 |
| 2096 | C | ALA | B | 242 | 19.265 | 78.460 | 49.037 | 1.00 | 106.40 |
| 2097 | O | ALA | B | 242 | 19.203 | 77.697 | 48.104 | 1.00 | 108.04 |
| 2098 | N | ALA | B | 243 | 18.207 | 78.966 | 49.715 | 1.00 | 105.11 |
| 2099 | CA | ALA | B | 243 | 16.768 | 78.714 | 49.496 | 1.00 | 101.10 |
| 2100 | CB | ALA | B | 243 | 16.502 | 77.309 | 49.423 | 1.00 | 97.27 |
| 2101 | C | ALA | B | 243 | 16.103 | 79.335 | 50.796 | 1.00 | 101.77 |
| 2102 | O | ALA | B | 243 | 16.810 | 79.916 | 51.666 | 1.00 | 101.06 |
| 2103 | N | ALA | B | 244 | 14.761 | 79.338 | 50.863 | 1.00 | 100.84 |
| 2104 | CA | ALA | B | 244 | 13.907 | 79.821 | 51.969 | 1.00 | 99.77 |
| 2105 | CB | ALA | B | 244 | 14.063 | 78.822 | 53.233 | 1.00 | 100.95 |
| 2106 | C | ALA | B | 244 | 13.795 | 81.316 | 52.457 | 1.00 | 101.34 |
| 2107 | O | ALA | B | 244 | 13.050 | 82.166 | 51.858 | 1.00 | 100.88 |
| 2108 | N | ALA | B | 245 | 14.517 | 81.556 | 53.593 | 1.00 | 102.46 |
| 2109 | CA | ALA | B | 245 | 14.675 | 82.740 | 54.351 | 1.00 | 101.68 |
| 2110 | CB | ALA | B | 245 | 15.956 | 83.504 | 53.854 | 1.00 | 103.15 |
| 2111 | C | ALA | B | 245 | 13.282 | 83.600 | 54.522 | 1.00 | 102.07 |
| 2112 | O | ALA | B | 245 | 12.242 | 83.072 | 55.091 | 1.00 | 98.13 |
| 2113 | N | ALA | B | 246 | 13.313 | 84.906 | 54.167 | 1.00 | 103.68 |
| 2114 | CA | ALA | B | 246 | 12.174 | 85.963 | 54.354 | 1.00 | 106.15 |
| 2115 | CB | ALA | B | 246 | 11.254 | 85.604 | 55.581 | 1.00 | 107.17 |
| 2116 | C | ALA | B | 246 | 12.768 | 87.336 | 54.611 | 1.00 | 103.67 |
| 2117 | O | ALA | B | 246 | 13.973 | 87.382 | 54.983 | 1.00 | 106.48 |
| 2118 | N | GLN | B | 266 | 6.953 | 87.068 | 62.544 | 1.00 | 79.67 |
| 2119 | CA | GLN | B | 266 | 7.541 | 87.520 | 61.272 | 1.00 | 87.37 |
| 2120 | CB | GLN | B | 266 | 7.673 | 89.025 | 61.102 | 1.00 | 88.42 |
| 2121 | CG | GLN | B | 266 | 6.268 | 89.732 | 60.996 | 1.00 | 98.96 |
| 2122 | CD | GLN | B | 266 | 5.890 | 90.602 | 62.314 | 1.00 | 92.78 |
| 2123 | OE1 | GLN | B | 266 | 5.183 | 91.529 | 62.175 | 1.00 | 86.17 |
| 2124 | NE2 | GLN | B | 266 | 6.550 | 90.324 | 63.519 | 1.00 | 92.39 |
| 2125 | C | GLN | B | 266 | 8.981 | 87.127 | 61.449 | 1.00 | 87.17 |
| 2126 | O | GLN | B | 266 | 9.512 | 86.310 | 60.885 | 1.00 | 83.42 |
| 2127 | N | ALA | B | 267 | 9.640 | 87.770 | 62.327 | 1.00 | 91.53 |
| 2128 | CA | ALA | B | 267 | 11.131 | 87.476 | 62.636 | 1.00 | 95.36 |
| 2129 | CB | ALA | B | 267 | 11.605 | 88.595 | 63.456 | 1.00 | 94.72 |
| 2130 | C | ALA | B | 267 | 11.491 | 86.138 | 63.438 | 1.00 | 92.03 |
| 2131 | O | ALA | B | 267 | 12.383 | 85.532 | 63.473 | 1.00 | 90.82 |
| 2132 | N | PHE | B | 268 | 10.539 | 85.702 | 64.144 | 1.00 | 92.21 |
| 2133 | CA | PHE | B | 268 | 10.383 | 84.455 | 64.990 | 1.00 | 86.22 |
| 2134 | CB | PHE | B | 268 | 9.005 | 84.449 | 65.761 | 1.00 | 89.59 |
| 2135 | CG | PHE | B | 268 | 8.833 | 83.386 | 66.595 | 1.00 | 90.51 |
| 2136 | CD1 | PHE | B | 268 | 9.929 | 83.048 | 67.309 | 1.00 | 98.22 |
| 2137 | CE1 | PHE | B | 268 | 9.897 | 81.943 | 68.142 | 1.00 | 93.68 |
| 2138 | CZ | PHE | B | 268 | 8.822 | 81.106 | 68.218 | 1.00 | 91.59 |
| 2139 | CE2 | PHE | B | 268 | 7.702 | 81.414 | 67.511 | 1.00 | 94.27 |
| 2140 | CD2 | PHE | B | 268 | 7.733 | 82.598 | 66.642 | 1.00 | 87.97 |
| 2141 | C | PHE | B | 268 | 10.531 | 83.323 | 64.071 | 1.00 | 84.42 |
| 2142 | O | PHE | B | 268 | 10.531 | 82.320 | 64.553 | 1.00 | 85.87 |
| 2143 | N | ALA | B | 269 | 10.425 | 83.506 | 62.776 | 1.00 | 82.21 |
| 2144 | CA | ALA | B | 269 | 10.531 | 82.387 | 61.808 | 1.00 | 79.55 |
| 2145 | CB | ALA | B | 269 | 9.447 | 82.574 | 60.559 | 1.00 | 86.99 |
| 2146 | C | ALA | B | 269 | 11.777 | 82.572 | 61.267 | 1.00 | 80.77 |
| 2147 | O | ALA | B | 269 | 12.488 | 81.571 | 60.924 | 1.00 | 80.47 |
| 2148 | N | HIS | B | 270 | 12.177 | 83.871 | 61.124 | 1.00 | 80.30 |
| 2149 | CA | HIS | B | 270 | 13.498 | 84.074 | 60.549 | 1.00 | 81.61 |
| 2150 | CB | HIS | B | 270 | 13.981 | 85.474 | 60.648 | 1.00 | 79.26 |
| 2151 | CG | HIS | B | 270 | 15.397 | 85.686 | 60.208 | 1.00 | 79.92 |
| 2152 | ND1 | HIS | B | 270 | 15.858 | 85.338 | 58.935 | 1.00 | 88.96 |
| 2153 | CE1 | HIS | B | 270 | 17.167 | 85.567 | 58.854 | 1.00 | 89.00 |
| 2154 | NE2 | HIS | B | 270 | 17.547 | 86.143 | 59.990 | 1.00 | 80.69 |
| 2155 | CD2 | HIS | B | 270 | 16.464 | 86.261 | 60.826 | 1.00 | 79.78 |
| 2156 | C | HIS | B | 270 | 14.364 | 83.169 | 61.501 | 1.00 | 77.66 |
| 2157 | O | HIS | B | 270 | 15.185 | 82.332 | 60.986 | 1.00 | 82.44 |
| 2158 | N | PHE | B | 271 | 14.167 | 83.269 | 62.823 | 1.00 | 73.18 |
| 2159 | CA | PHE | B | 271 | 15.350 | 82.615 | 63.646 | 1.00 | 80.03 |
| 2160 | CB | PHE | B | 271 | 15.176 | 83.094 | 65.106 | 1.00 | 77.22 |
| 2161 | CG | PHE | B | 271 | 16.097 | 84.140 | 65.441 | 1.00 | 84.35 |
| 2162 | CD1 | PHE | B | 271 | 17.573 | 83.924 | 65.446 | 1.00 | 84.34 |
| 2163 | CE1 | PHE | B | 271 | 18.374 | 85.069 | 65.878 | 1.00 | 86.87 |
| 2164 | CZ | PHE | B | 271 | 17.677 | 86.291 | 66.095 | 1.00 | 76.68 |
| 2165 | CE2 | PHE | B | 271 | 16.370 | 86.438 | 65.851 | 1.00 | 77.21 |
| 2166 | CD2 | PHE | B | 271 | 15.582 | 85.407 | 65.679 | 1.00 | 80.51 |
| 2167 | C | PHE | B | 271 | 15.250 | 81.086 | 63.568 | 1.00 | 75.88 |
| 2168 | O | PHE | B | 271 | 16.151 | 80.246 | 63.317 | 1.00 | 77.81 |
| 2169 | N | THR | B | 272 | 13.980 | 80.823 | 63.795 | 1.00 | 74.79 |
| 2170 | CA | THR | B | 272 | 13.200 | 79.536 | 63.756 | 1.00 | 73.99 |
| 2171 | CB | THR | B | 272 | 11.591 | 79.623 | 63.982 | 1.00 | 74.69 |
| 2172 | OG1 | THR | B | 272 | 11.267 | 80.058 | 65.296 | 1.00 | 78.22 |
| 2173 | CG2 | THR | B | 272 | 11.131 | 78.270 | 63.971 | 1.00 | 82.08 |
| 2174 | C | THR | B | 272 | 13.424 | 78.880 | 62.405 | 1.00 | 68.81 |
| 2175 | O | THR | B | 272 | 13.336 | 77.663 | 62.264 | 1.00 | 73.54 |
| 2176 | N | GLU | B | 273 | 13.892 | 79.676 | 61.456 | 1.00 | 68.43 |
| 2177 | CA | GLU | B | 273 | 14.180 | 79.135 | 60.200 | 1.00 | 69.62 |
| 2178 | CB | GLU | B | 273 | 13.896 | 80.301 | 59.176 | 1.00 | 73.97 |
| 2179 | CG | GLU | B | 273 | 12.639 | 80.045 | 58.219 | 1.00 | 78.18 |
| 2180 | CD | GLU | B | 273 | 12.078 | 81.415 | 57.497 | 1.00 | 78.36 |
| 2181 | OE1 | GLU | B | 273 | 11.442 | 82.220 | 58.142 | 1.00 | 71.56 |
| 2182 | OE2 | GLU | B | 273 | 12.284 | 81.645 | 56.297 | 1.00 | 74.81 |
| 2183 | C | GLU | B | 273 | 15.593 | 78.771 | 60.181 | 1.00 | 66.71 |
| 2184 | O | GLU | B | 273 | 16.091 | 77.764 | 59.765 | 1.00 | 64.57 |
| 2185 | N | LEU | B | 274 | 16.288 | 79.719 | 60.680 | 1.00 | 65.82 |
| 2186 | CA | LEU | B | 274 | 17.682 | 79.427 | 60.696 | 1.00 | 69.97 |
| 2187 | CB | LEU | B | 274 | 18.459 | 80.686 | 61.251 | 1.00 | 78.70 |
| 2188 | CG | LEU | B | 274 | 20.159 | 80.548 | 61.290 | 1.00 | 71.61 |
| 2189 | CD1 | LEU | B | 274 | 20.909 | 80.170 | 59.963 | 1.00 | 84.54 |
| 2190 | CD2 | LEU | B | 274 | 20.826 | 81.824 | 61.614 | 1.00 | 88.89 |
| 2191 | C | LEU | B | 274 | 18.164 | 78.304 | 61.694 | 1.00 | 63.14 |
| 2192 | O | LEU | B | 274 | 19.076 | 77.590 | 61.486 | 1.00 | 53.88 |
| 2193 | N | ALA | B | 275 | 17.421 | 78.053 | 62.713 | 1.00 | 63.35 |
| 2194 | CA | ALA | B | 275 | 17.751 | 76.963 | 63.565 | 1.00 | 63.41 |
| 2195 | CB | ALA | B | 275 | 16.697 | 77.133 | 64.830 | 1.00 | 61.70 |
| 2196 | C | ALA | B | 275 | 17.487 | 75.617 | 62.756 | 1.00 | 65.82 |
| 2197 | O | ALA | B | 275 | 18.409 | 74.716 | 62.871 | 1.00 | 59.36 |
| 2198 | N | ILE | B | 276 | 16.291 | 75.527 | 61.979 | 1.00 | 60.42 |
| 2199 | CA | ILE | B | 276 | 15.967 | 74.404 | 61.210 | 1.00 | 56.65 |
| 2200 | CB | ILE | B | 276 | 14.585 | 74.566 | 60.433 | 1.00 | 53.48 |
| 2201 | CG1 | ILE | B | 276 | 13.491 | 74.529 | 61.520 | 1.00 | 60.73 |
| 2202 | CD1 | ILE | B | 276 | 11.929 | 74.884 | 61.183 | 1.00 | 73.36 |
| 2203 | CG2 | ILE | B | 276 | 14.351 | 73.484 | 59.499 | 1.00 | 46.80 |
| 2204 | C | ILE | B | 276 | 16.994 | 74.036 | 60.418 | 1.00 | 55.64 |
| 2205 | O | ILE | B | 276 | 17.098 | 72.909 | 60.058 | 1.00 | 65.41 |
| 2206 | N | ILE | B | 277 | 17.822 | 74.899 | 59.999 | 1.00 | 58.61 |
| 2207 | CA | ILE | B | 277 | 19.026 | 74.628 | 59.151 | 1.00 | 54.52 |
| 2208 | CB | ILE | B | 277 | 19.458 | 76.021 | 58.603 | 1.00 | 58.00 |
| 2209 | CG1 | ILE | B | 277 | 18.132 | 76.618 | 58.041 | 1.00 | 58.20 |
| 2210 | CD1 | ILE | B | 277 | 18.060 | 78.023 | 58.363 | 1.00 | 67.84 |
| 2211 | CG2 | ILE | B | 277 | 20.582 | 75.792 | 57.634 | 1.00 | 62.11 |
| 2212 | C | ILE | B | 277 | 20.028 | 74.087 | 59.838 | 1.00 | 55.62 |
| 2213 | O | ILE | B | 277 | 20.757 | 73.255 | 59.425 | 1.00 | 58.46 |
| 2214 | N | SER | B | 278 | 20.165 | 74.668 | 61.119 | 1.00 | 55.68 |
| 2215 | CA | SER | B | 278 | 21.243 | 74.182 | 62.080 | 1.00 | 54.04 |
| 2216 | CB | SER | B | 278 | 21.279 | 74.978 | 63.347 | 1.00 | 61.52 |
| 2217 | OG | SER | B | 278 | 20.704 | 76.195 | 63.076 | 1.00 | 73.03 |
| 2218 | C | SER | B | 278 | 21.197 | 72.681 | 62.521 | 1.00 | 44.66 |
| 2219 | O | SER | B | 278 | 21.971 | 71.913 | 62.631 | 1.00 | 46.18 |
| 2220 | N | VAL | B | 279 | 19.911 | 72.306 | 62.666 | 1.00 | 51.44 |
| 2221 | CA | VAL | B | 279 | 19.498 | 70.964 | 63.038 | 1.00 | 49.70 |
| 2222 | CB | VAL | B | 279 | 18.252 | 70.872 | 63.069 | 1.00 | 56.96 |
| 2223 | CG1 | VAL | B | 279 | 17.285 | 69.449 | 63.474 | 1.00 | 60.86 |
| 2224 | CG2 | VAL | B | 279 | 17.405 | 71.876 | 64.073 | 1.00 | 58.26 |
| 2225 | C | VAL | B | 279 | 19.959 | 69.973 | 62.052 | 1.00 | 50.33 |
| 2226 | O | VAL | B | 279 | 20.198 | 68.818 | 62.507 | 1.00 | 50.44 |
| 2227 | N | GLN | B | 280 | 20.179 | 70.355 | 60.775 | 1.00 | 54.00 |
| 2228 | CA | GLN | B | 280 | 20.766 | 69.299 | 59.768 | 1.00 | 50.44 |
| 2229 | CB | GLN | B | 280 | 20.317 | 69.830 | 58.243 | 1.00 | 58.81 |
| 2230 | CG | GLN | B | 280 | 21.341 | 68.972 | 57.208 | 1.00 | 52.24 |
| 2231 | CD | GLN | B | 280 | 20.545 | 67.504 | 57.051 | 1.00 | 60.94 |
| 2232 | OE1 | GLN | B | 280 | 19.444 | 67.460 | 56.472 | 1.00 | 79.31 |
| 2233 | NE2 | GLN | B | 280 | 21.147 | 66.474 | 57.368 | 1.00 | 47.82 |
| 2234 | C | GLN | B | 280 | 22.137 | 69.264 | 59.730 | 1.00 | 55.32 |
| 2235 | O | GLN | B | 280 | 22.877 | 68.086 | 59.623 | 1.00 | 54.41 |
| 2236 | N | GLU | B | 281 | 22.822 | 70.548 | 59.851 | 1.00 | 57.18 |
| 2237 | CA | GLU | B | 281 | 24.342 | 70.372 | 59.794 | 1.00 | 51.39 |
| 2238 | CB | GLU | B | 281 | 24.689 | 71.870 | 59.981 | 1.00 | 60.52 |
| 2239 | CG | GLU | B | 281 | 23.537 | 72.847 | 59.755 | 1.00 | 65.49 |
| 2240 | CD | GLU | B | 281 | 23.967 | 74.410 | 60.323 | 1.00 | 81.25 |
| 2241 | OE1 | GLU | B | 281 | 24.406 | 74.654 | 61.490 | 1.00 | 76.30 |
| 2242 | OE2 | GLU | B | 281 | 23.880 | 75.392 | 59.525 | 1.00 | 73.76 |
| 2243 | C | GLU | B | 281 | 24.651 | 69.620 | 60.970 | 1.00 | 48.92 |
| 2244 | O | GLU | B | 281 | 25.664 | 69.008 | 61.028 | 1.00 | 56.76 |
| 2245 | N | ILE | B | 282 | 23.794 | 69.627 | 62.092 | 1.00 | 52.05 |
| 2246 | CA | ILE | B | 282 | 24.445 | 68.874 | 63.277 | 1.00 | 57.49 |
| 2247 | CB | ILE | B | 282 | 23.393 | 69.292 | 64.536 | 1.00 | 54.00 |
| 2248 | CG1 | ILE | B | 282 | 23.998 | 70.716 | 65.057 | 1.00 | 54.53 |
| 2249 | CD1 | ILE | B | 282 | 23.111 | 71.419 | 65.835 | 1.00 | 49.35 |
| 2250 | CG2 | ILE | B | 282 | 23.495 | 68.340 | 65.377 | 1.00 | 45.45 |
| 2251 | C | ILE | B | 282 | 24.212 | 67.317 | 63.085 | 1.00 | 54.21 |
| 2252 | O | ILE | B | 282 | 25.343 | 66.575 | 63.214 | 1.00 | 53.24 |
| 2253 | N | VAL | B | 283 | 23.089 | 66.821 | 62.692 | 1.00 | 50.83 |
| 2254 | CA | VAL | B | 283 | 22.999 | 65.423 | 62.436 | 1.00 | 56.24 |
| 2255 | CB | VAL | B | 283 | 21.731 | 65.072 | 61.917 | 1.00 | 58.66 |
| 2256 | CG1 | VAL | B | 283 | 21.849 | 63.626 | 61.445 | 1.00 | 54.04 |
| 2257 | CG2 | VAL | B | 283 | 20.315 | 65.468 | 62.959 | 1.00 | 50.38 |
| 2258 | C | VAL | B | 283 | 23.943 | 64.939 | 61.410 | 1.00 | 56.20 |
| 2259 | O | VAL | B | 283 | 24.523 | 63.848 | 61.557 | 1.00 | 51.77 |
| 2260 | N | ASP | B | 284 | 24.292 | 65.778 | 60.472 | 1.00 | 55.22 |
| 2261 | CA | ASP | B | 284 | 25.346 | 65.211 | 59.571 | 1.00 | 64.25 |
| 2262 | CB | ASP | B | 284 | 25.303 | 65.906 | 58.091 | 1.00 | 68.50 |
| 2263 | CG | ASP | B | 284 | 24.065 | 65.417 | 57.282 | 1.00 | 68.12 |
| 2264 | OD1 | ASP | B | 284 | 23.881 | 64.122 | 57.296 | 1.00 | 67.53 |
| 2265 | OD2 | ASP | B | 284 | 23.246 | 66.294 | 56.739 | 1.00 | 68.04 |
| 2266 | C | ASP | B | 284 | 26.612 | 65.167 | 60.047 | 1.00 | 63.92 |
| 2267 | O | ASP | B | 284 | 27.455 | 64.571 | 59.436 | 1.00 | 62.17 |
| 2268 | N | PHE | B | 285 | 26.849 | 66.018 | 61.097 | 1.00 | 64.71 |
| 2269 | CA | PHE | B | 285 | 28.289 | 65.938 | 61.652 | 1.00 | 61.65 |
| 2270 | CB | PHE | B | 285 | 28.516 | 67.217 | 62.474 | 1.00 | 63.46 |
| 2271 | CG | PHE | B | 285 | 29.633 | 67.192 | 63.322 | 1.00 | 57.88 |
| 2272 | CD1 | PHE | B | 285 | 30.710 | 67.208 | 62.677 | 1.00 | 62.04 |
| 2273 | CE1 | PHE | B | 285 | 32.294 | 67.127 | 63.392 | 1.00 | 65.42 |
| 2274 | CZ | PHE | B | 285 | 32.081 | 67.078 | 64.810 | 1.00 | 70.04 |
| 2275 | CE2 | PHE | B | 285 | 30.787 | 67.037 | 65.384 | 1.00 | 55.03 |
| 2276 | CD2 | PHE | B | 285 | 29.605 | 67.142 | 64.725 | 1.00 | 57.34 |
| 2277 | C | PHE | B | 285 | 28.250 | 64.787 | 62.666 | 1.00 | 57.92 |
| 2278 | O | PHE | B | 285 | 29.264 | 64.083 | 62.806 | 1.00 | 54.29 |
| 2279 | N | ALA | B | 286 | 26.974 | 64.544 | 63.356 | 1.00 | 62.27 |
| 2280 | CA | ALA | B | 286 | 27.063 | 63.462 | 64.418 | 1.00 | 56.88 |
| 2281 | CB | ALA | B | 286 | 25.756 | 63.582 | 65.092 | 1.00 | 55.00 |
| 2282 | C | ALA | B | 286 | 27.280 | 62.110 | 63.683 | 1.00 | 59.47 |
| 2283 | O | ALA | B | 286 | 28.273 | 61.334 | 64.050 | 1.00 | 59.93 |
| 2284 | N | LYS | B | 287 | 26.493 | 61.853 | 62.624 | 1.00 | 62.20 |
| 2285 | CA | LYS | B | 287 | 26.665 | 60.564 | 61.794 | 1.00 | 63.29 |
| 2286 | CB | LYS | B | 287 | 25.771 | 60.724 | 60.554 | 1.00 | 55.81 |
| 2287 | CG | LYS | B | 287 | 24.319 | 60.987 | 60.852 | 1.00 | 56.80 |
| 2288 | CD | LYS | B | 287 | 23.359 | 61.386 | 59.489 | 1.00 | 70.39 |
| 2289 | CE | LYS | B | 287 | 23.158 | 60.140 | 58.561 | 1.00 | 61.74 |
| 2290 | NZ | LYS | B | 287 | 22.811 | 60.223 | 57.189 | 1.00 | 60.01 |
| 2291 | C | LYS | B | 287 | 28.031 | 60.319 | 61.490 | 1.00 | 59.19 |
| 2292 | O | LYS | B | 287 | 28.487 | 59.291 | 61.648 | 1.00 | 57.30 |
| 2293 | N | GLN | B | 288 | 28.792 | 61.327 | 61.254 | 1.00 | 57.51 |
| 2294 | CA | GLN | B | 288 | 30.344 | 61.122 | 61.058 | 1.00 | 57.22 |
| 2295 | CB | GLN | B | 288 | 30.812 | 62.429 | 60.049 | 1.00 | 61.51 |
| 2296 | CG | GLN | B | 288 | 31.229 | 62.044 | 58.542 | 1.00 | 62.61 |
| 2297 | CD | GLN | B | 288 | 32.406 | 61.121 | 58.469 | 1.00 | 76.06 |
| 2298 | OE1 | GLN | B | 288 | 32.202 | 59.789 | 58.161 | 1.00 | 70.64 |
| 2299 | NE2 | GLN | B | 288 | 33.658 | 61.690 | 58.793 | 1.00 | 68.17 |
| 2300 | C | GLN | B | 288 | 31.051 | 61.018 | 62.019 | 1.00 | 54.37 |
| 2301 | O | GLN | B | 288 | 32.337 | 61.108 | 61.886 | 1.00 | 61.46 |
| 2302 | N | VAL | B | 289 | 30.419 | 61.010 | 63.190 | 1.00 | 61.78 |
| 2303 | CA | VAL | B | 289 | 31.531 | 60.817 | 64.348 | 1.00 | 58.20 |
| 2304 | CB | VAL | B | 289 | 30.984 | 61.685 | 65.746 | 1.00 | 63.17 |
| 2305 | CG1 | VAL | B | 289 | 32.033 | 61.537 | 66.877 | 1.00 | 56.94 |
| 2306 | CG2 | VAL | B | 289 | 30.675 | 63.103 | 65.519 | 1.00 | 53.71 |
| 2307 | C | VAL | B | 289 | 31.872 | 59.511 | 64.637 | 1.00 | 56.27 |
| 2308 | O | VAL | B | 289 | 31.115 | 58.717 | 64.937 | 1.00 | 67.55 |
| 2309 | N | PRO | B | 290 | 33.036 | 59.135 | 64.777 | 1.00 | 61.39 |
| 2310 | CA | PRO | B | 290 | 33.490 | 57.792 | 65.097 | 1.00 | 60.40 |
| 2311 | CB | PRO | B | 290 | 34.896 | 57.881 | 65.238 | 1.00 | 59.76 |
| 2312 | CG | PRO | B | 290 | 35.259 | 59.069 | 64.376 | 1.00 | 57.41 |
| 2313 | CD | PRO | B | 290 | 34.169 | 59.982 | 64.687 | 1.00 | 65.61 |
| 2314 | C | PRO | B | 290 | 32.877 | 57.106 | 66.282 | 1.00 | 62.07 |
| 2315 | O | PRO | B | 290 | 33.543 | 57.041 | 67.400 | 1.00 | 63.88 |
| 2316 | N | GLY | B | 291 | 31.787 | 56.458 | 66.045 | 1.00 | 53.48 |
| 2317 | CA | GLY | B | 291 | 31.118 | 55.692 | 67.264 | 1.00 | 52.08 |
| 2318 | C | GLY | B | 291 | 29.629 | 55.988 | 67.409 | 1.00 | 47.37 |
| 2319 | O | GLY | B | 291 | 28.827 | 55.279 | 67.830 | 1.00 | 47.64 |
| 2320 | N | PHE | B | 292 | 29.260 | 57.143 | 66.788 | 1.00 | 46.97 |
| 2321 | CA | PHE | B | 292 | 27.961 | 57.485 | 66.870 | 1.00 | 48.61 |
| 2322 | CB | PHE | B | 292 | 27.816 | 58.865 | 66.348 | 1.00 | 44.81 |
| 2323 | CG | PHE | B | 292 | 26.433 | 59.424 | 66.587 | 1.00 | 44.19 |
| 2324 | CD1 | PHE | B | 292 | 26.050 | 59.806 | 67.863 | 1.00 | 42.15 |
| 2325 | CE1 | PHE | B | 292 | 24.867 | 60.284 | 68.073 | 1.00 | 36.36 |
| 2326 | CZ | PHE | B | 292 | 23.924 | 60.297 | 67.124 | 1.00 | 41.35 |
| 2327 | CE2 | PHE | B | 292 | 24.222 | 59.911 | 65.844 | 1.00 | 44.46 |
| 2328 | CD2 | PHE | B | 292 | 25.541 | 59.565 | 65.559 | 1.00 | 42.70 |
| 2329 | C | PHE | B | 292 | 26.955 | 56.514 | 66.316 | 1.00 | 56.70 |
| 2330 | O | PHE | B | 292 | 25.969 | 56.113 | 67.050 | 1.00 | 56.57 |
| 2331 | N | LEU | B | 293 | 27.131 | 56.163 | 64.973 | 1.00 | 53.68 |
| 2332 | CA | LEU | B | 293 | 26.241 | 55.342 | 64.318 | 1.00 | 53.58 |
| 2333 | CB | LEU | B | 293 | 26.435 | 55.479 | 62.811 | 1.00 | 49.24 |
| 2334 | CG | LEU | B | 293 | 25.992 | 56.784 | 62.295 | 1.00 | 53.64 |
| 2335 | CD1 | LEU | B | 293 | 26.253 | 56.997 | 60.850 | 1.00 | 53.77 |
| 2336 | CD2 | LEU | B | 293 | 24.184 | 56.923 | 62.401 | 1.00 | 48.39 |
| 2337 | C | LEU | B | 293 | 26.103 | 53.878 | 64.768 | 1.00 | 52.40 |
| 2338 | O | LEU | B | 293 | 25.204 | 53.115 | 64.277 | 1.00 | 52.95 |
| 2339 | N | GLN | B | 294 | 27.063 | 53.528 | 65.590 | 1.00 | 54.66 |
| 2340 | CA | GLN | B | 294 | 27.133 | 52.177 | 66.240 | 1.00 | 58.82 |
| 2341 | CB | GLN | B | 294 | 28.534 | 51.766 | 66.446 | 1.00 | 55.99 |
| 2342 | CG | GLN | B | 294 | 29.381 | 52.586 | 65.434 | 1.00 | 61.48 |
| 2343 | CD | GLN | B | 294 | 30.925 | 52.325 | 65.623 | 1.00 | 74.17 |
| 2344 | OE1 | GLN | B | 294 | 31.319 | 51.724 | 66.545 | 1.00 | 75.85 |
| 2345 | NE2 | GLN | B | 294 | 31.784 | 52.888 | 64.776 | 1.00 | 69.33 |
| 2346 | C | GLN | B | 294 | 26.389 | 52.057 | 67.524 | 1.00 | 58.14 |
| 2347 | O | GLN | B | 294 | 26.326 | 51.003 | 68.038 | 1.00 | 61.97 |
| 2348 | N | LEU | B | 295 | 25.691 | 53.044 | 67.947 | 1.00 | 56.46 |
| 2349 | CA | LEU | B | 295 | 25.025 | 52.982 | 69.224 | 1.00 | 61.54 |
| 2350 | CB | LEU | B | 295 | 24.900 | 54.500 | 69.876 | 1.00 | 55.24 |
| 2351 | CG | LEU | B | 295 | 26.176 | 55.068 | 70.533 | 1.00 | 50.71 |
| 2352 | CD1 | LEU | B | 295 | 26.052 | 56.510 | 70.987 | 1.00 | 36.73 |
| 2353 | CD2 | LEU | B | 295 | 26.341 | 54.250 | 71.781 | 1.00 | 48.13 |
| 2354 | C | LEU | B | 295 | 23.605 | 52.533 | 68.800 | 1.00 | 60.78 |
| 2355 | O | LEU | B | 295 | 23.213 | 52.803 | 67.636 | 1.00 | 59.88 |
| 2356 | N | GLY | B | 296 | 22.742 | 52.176 | 69.774 | 1.00 | 59.07 |
| 2357 | CA | GLY | B | 296 | 21.359 | 51.909 | 69.435 | 1.00 | 55.12 |
| 2358 | C | GLY | B | 296 | 20.749 | 53.114 | 68.818 | 1.00 | 62.84 |
| 2359 | O | GLY | B | 296 | 21.137 | 54.384 | 69.095 | 1.00 | 60.06 |
| 2360 | N | ARG | B | 297 | 19.716 | 52.882 | 67.977 | 1.00 | 63.08 |
| 2361 | CA | ARG | B | 297 | 19.063 | 54.044 | 67.321 | 1.00 | 62.49 |
| 2362 | CB | ARG | B | 297 | 17.986 | 53.603 | 66.339 | 1.00 | 55.91 |
| 2363 | CG | ARG | B | 297 | 16.766 | 54.303 | 66.499 | 1.00 | 61.42 |
| 2364 | CD | ARG | B | 297 | 15.836 | 54.353 | 65.183 | 1.00 | 66.08 |
| 2365 | NE | ARG | B | 297 | 16.781 | 54.841 | 64.113 | 1.00 | 83.98 |
| 2366 | CZ | ARG | B | 297 | 16.394 | 55.084 | 62.853 | 1.00 | 76.13 |
| 2367 | NH1 | ARG | B | 297 | 15.378 | 54.775 | 62.444 | 1.00 | 84.00 |
| 2368 | NH2 | ARG | B | 297 | 17.227 | 55.522 | 61.910 | 1.00 | 68.07 |
| 2369 | C | ARG | B | 297 | 18.499 | 54.957 | 68.436 | 1.00 | 61.26 |
| 2370 | O | ARG | B | 297 | 18.481 | 56.167 | 68.385 | 1.00 | 63.34 |
| 2371 | N | GLU | B | 298 | 18.073 | 54.391 | 69.440 | 1.00 | 58.48 |
| 2372 | CA | GLU | B | 298 | 17.405 | 55.199 | 70.428 | 1.00 | 62.30 |
| 2373 | CB | GLU | B | 298 | 16.647 | 54.195 | 71.537 | 1.00 | 57.17 |
| 2374 | CG | GLU | B | 298 | 15.169 | 54.174 | 71.324 | 1.00 | 67.57 |
| 2375 | CD | GLU | B | 298 | 14.832 | 53.500 | 69.965 | 1.00 | 80.94 |
| 2376 | OE1 | GLU | B | 298 | 15.469 | 52.375 | 69.667 | 1.00 | 84.64 |
| 2377 | OE2 | GLU | B | 298 | 14.023 | 54.023 | 69.092 | 1.00 | 81.00 |
| 2378 | C | GLU | B | 298 | 18.476 | 56.177 | 71.163 | 1.00 | 51.80 |
| 2379 | O | GLU | B | 298 | 18.051 | 57.151 | 71.560 | 1.00 | 49.60 |
| 2380 | N | ASP | B | 299 | 19.539 | 55.564 | 71.522 | 1.00 | 47.86 |
| 2381 | CA | ASP | B | 299 | 20.497 | 56.312 | 72.187 | 1.00 | 51.84 |
| 2382 | CB | ASP | B | 299 | 21.629 | 55.407 | 72.765 | 1.00 | 45.01 |
| 2383 | CG | ASP | B | 299 | 21.158 | 54.667 | 73.927 | 1.00 | 44.83 |
| 2384 | OD1 | ASP | B | 299 | 20.149 | 55.035 | 74.559 | 1.00 | 45.60 |
| 2385 | OD2 | ASP | B | 299 | 21.877 | 53.829 | 74.379 | 1.00 | 48.45 |
| 2386 | C | ASP | B | 299 | 21.047 | 57.463 | 71.272 | 1.00 | 52.94 |
| 2387 | O | ASP | B | 299 | 21.683 | 58.425 | 71.778 | 1.00 | 52.05 |
| 2388 | N | GLN | B | 300 | 20.901 | 57.196 | 69.969 | 1.00 | 50.18 |
| 2389 | CA | GLN | B | 300 | 21.413 | 58.153 | 68.979 | 1.00 | 55.61 |
| 2390 | CB | GLN | B | 300 | 21.591 | 57.640 | 67.491 | 1.00 | 52.76 |
| 2391 | CG | GLN | B | 300 | 22.617 | 56.690 | 67.374 | 1.00 | 55.34 |
| 2392 | CD | GLN | B | 300 | 22.653 | 56.046 | 65.932 | 1.00 | 63.28 |
| 2393 | OE1 | GLN | B | 300 | 23.422 | 55.074 | 65.703 | 1.00 | 52.87 |
| 2394 | NE2 | GLN | B | 300 | 21.958 | 56.692 | 65.000 | 1.00 | 60.94 |
| 2395 | C | GLN | B | 300 | 20.536 | 59.360 | 68.892 | 1.00 | 51.50 |
| 2396 | O | GLN | B | 300 | 21.095 | 60.490 | 69.012 | 1.00 | 53.21 |
| 2397 | N | ILE | B | 301 | 19.309 | 59.133 | 69.107 | 1.00 | 43.21 |
| 2398 | CA | ILE | B | 301 | 18.474 | 60.115 | 69.204 | 1.00 | 44.82 |
| 2399 | CB | ILE | B | 301 | 16.916 | 59.617 | 68.821 | 1.00 | 44.76 |
| 2400 | CG1 | ILE | B | 301 | 15.936 | 60.581 | 69.007 | 1.00 | 41.61 |
| 2401 | CD1 | ILE | B | 301 | 14.749 | 60.228 | 69.501 | 1.00 | 46.97 |
| 2402 | CG2 | ILE | B | 301 | 16.727 | 58.642 | 69.732 | 1.00 | 50.79 |
| 2403 | C | ILE | B | 301 | 18.420 | 60.868 | 70.494 | 1.00 | 41.63 |
| 2404 | O | ILE | B | 301 | 18.064 | 62.079 | 70.638 | 1.00 | 35.91 |
| 2405 | N | ALA | B | 302 | 18.534 | 60.080 | 71.496 | 1.00 | 39.00 |
| 2406 | CA | ALA | B | 302 | 18.488 | 60.611 | 72.876 | 1.00 | 38.93 |
| 2407 | CB | ALA | B | 302 | 18.648 | 59.533 | 73.816 | 1.00 | 44.04 |
| 2408 | C | ALA | B | 302 | 19.757 | 61.609 | 73.031 | 1.00 | 39.30 |
| 2409 | O | ALA | B | 302 | 19.483 | 62.764 | 73.349 | 1.00 | 39.22 |
| 2410 | N | LEU | B | 303 | 20.888 | 61.185 | 72.606 | 1.00 | 38.64 |
| 2411 | CA | LEU | B | 303 | 22.029 | 61.991 | 72.654 | 1.00 | 45.39 |
| 2412 | CB | LEU | B | 303 | 23.367 | 61.219 | 72.213 | 1.00 | 42.55 |
| 2413 | CG | LEU | B | 303 | 23.701 | 60.032 | 73.096 | 1.00 | 37.97 |
| 2414 | CD1 | LEU | B | 303 | 25.083 | 59.516 | 72.592 | 1.00 | 28.68 |
| 2415 | CD2 | LEU | B | 303 | 23.953 | 60.625 | 74.516 | 1.00 | 29.84 |
| 2416 | C | LEU | B | 303 | 21.996 | 63.289 | 71.725 | 1.00 | 45.65 |
| 2417 | O | LEU | B | 303 | 22.299 | 64.460 | 72.187 | 1.00 | 40.78 |
| 2418 | N | LEU | B | 304 | 21.471 | 63.118 | 70.525 | 1.00 | 43.87 |
| 2419 | CA | LEU | B | 304 | 21.340 | 64.276 | 69.612 | 1.00 | 40.36 |
| 2420 | CB | LEU | B | 304 | 21.053 | 63.778 | 68.207 | 1.00 | 40.77 |
| 2421 | CG | LEU | B | 304 | 21.675 | 64.382 | 67.012 | 1.00 | 40.36 |
| 2422 | CD1 | LEU | B | 304 | 23.281 | 64.959 | 67.303 | 1.00 | 54.16 |
| 2423 | CD2 | LEU | B | 304 | 21.898 | 63.180 | 66.176 | 1.00 | 42.16 |
| 2424 | C | LEU | B | 304 | 20.229 | 65.320 | 70.153 | 1.00 | 38.75 |
| 2425 | O | LEU | B | 304 | 20.437 | 66.483 | 70.014 | 1.00 | 36.35 |
| 2426 | N | LYS | B | 305 | 19.163 | 64.828 | 70.594 | 1.00 | 37.85 |
| 2427 | CA | LYS | B | 305 | 18.097 | 65.705 | 71.029 | 1.00 | 45.89 |
| 2428 | CB | LYS | B | 305 | 16.814 | 64.982 | 71.652 | 1.00 | 44.35 |
| 2429 | CG | LYS | B | 305 | 15.700 | 64.899 | 70.682 | 1.00 | 61.08 |
| 2430 | CD | LYS | B | 305 | 14.407 | 64.197 | 71.605 | 1.00 | 60.08 |
| 2431 | CE | LYS | B | 305 | 13.300 | 64.282 | 70.594 | 1.00 | 64.90 |
| 2432 | NZ | LYS | B | 305 | 12.007 | 63.803 | 71.012 | 1.00 | 69.57 |
| 2433 | C | LYS | B | 305 | 18.599 | 66.663 | 72.127 | 1.00 | 46.48 |
| 2434 | O | LYS | B | 305 | 18.021 | 67.794 | 72.370 | 1.00 | 42.93 |
| 2435 | N | ALA | B | 306 | 19.386 | 66.009 | 73.098 | 1.00 | 44.50 |
| 2436 | CA | ALA | B | 306 | 19.806 | 66.844 | 74.329 | 1.00 | 40.48 |
| 2437 | CB | ALA | B | 306 | 20.225 | 65.957 | 75.470 | 1.00 | 40.95 |
| 2438 | C | ALA | B | 306 | 20.937 | 67.763 | 73.946 | 1.00 | 42.42 |
| 2439 | O | ALA | B | 306 | 21.034 | 68.874 | 74.591 | 1.00 | 41.21 |
| 2440 | N | SER | B | 307 | 21.821 | 67.339 | 72.987 | 1.00 | 38.22 |
| 2441 | CA | SER | B | 307 | 22.925 | 68.166 | 72.715 | 1.00 | 43.23 |
| 2442 | CB | SER | B | 307 | 24.204 | 67.314 | 72.686 | 1.00 | 42.01 |
| 2443 | OG | SER | B | 307 | 24.360 | 66.547 | 71.741 | 1.00 | 42.17 |
| 2444 | C | SER | B | 307 | 22.856 | 69.187 | 71.561 | 1.00 | 44.87 |
| 2445 | O | SER | B | 307 | 23.725 | 69.994 | 71.374 | 1.00 | 42.46 |
| 2446 | N | THR | B | 308 | 21.653 | 69.113 | 70.911 | 1.00 | 45.36 |
| 2447 | CA | THR | B | 308 | 21.517 | 69.993 | 69.685 | 1.00 | 45.53 |
| 2448 | CB | THR | B | 308 | 19.923 | 69.707 | 69.135 | 1.00 | 48.08 |
| 2449 | OG1 | THR | B | 308 | 20.273 | 68.570 | 68.313 | 1.00 | 48.56 |
| 2450 | CG2 | THR | B | 308 | 19.445 | 70.978 | 68.212 | 1.00 | 43.38 |
| 2451 | C | THR | B | 308 | 21.632 | 71.579 | 70.125 | 1.00 | 39.58 |
| 2452 | O | THR | B | 308 | 22.534 | 72.152 | 69.585 | 1.00 | 38.96 |
| 2453 | N | ILE | B | 309 | 20.758 | 72.042 | 70.902 | 1.00 | 39.26 |
| 2454 | CA | ILE | B | 309 | 20.846 | 73.393 | 71.489 | 1.00 | 37.98 |
| 2455 | CB | ILE | B | 309 | 19.882 | 73.642 | 72.397 | 1.00 | 37.29 |
| 2456 | CG1 | ILE | B | 309 | 19.695 | 75.107 | 72.719 | 1.00 | 42.58 |
| 2457 | CD1 | ILE | B | 309 | 19.585 | 75.887 | 71.264 | 1.00 | 48.19 |
| 2458 | CG2 | ILE | B | 309 | 19.977 | 72.945 | 73.613 | 1.00 | 41.10 |
| 2459 | C | ILE | B | 309 | 22.088 | 73.772 | 72.134 | 1.00 | 41.77 |
| 2460 | O | ILE | B | 309 | 22.443 | 74.978 | 72.097 | 1.00 | 39.52 |
| 2461 | N | GLU | B | 310 | 22.977 | 72.761 | 72.526 | 1.00 | 37.45 |
| 2462 | CA | GLU | B | 310 | 24.210 | 73.186 | 73.072 | 1.00 | 39.85 |
| 2463 | CB | GLU | B | 310 | 24.799 | 72.120 | 74.300 | 1.00 | 31.33 |
| 2464 | CG | GLU | B | 310 | 23.692 | 71.853 | 75.284 | 1.00 | 33.40 |
| 2465 | CD | GLU | B | 310 | 24.089 | 70.879 | 76.384 | 1.00 | 26.27 |
| 2466 | OE1 | GLU | B | 310 | 23.256 | 70.555 | 77.138 | 1.00 | 32.27 |
| 2467 | OE2 | GLU | B | 310 | 25.214 | 70.462 | 76.402 | 1.00 | 33.68 |
| 2468 | C | GLU | B | 310 | 25.093 | 73.360 | 72.066 | 1.00 | 40.80 |
| 2469 | O | GLU | B | 310 | 26.168 | 74.152 | 72.192 | 1.00 | 42.61 |
| 2470 | N | ILE | B | 311 | 25.157 | 72.387 | 71.084 | 1.00 | 40.99 |
| 2471 | CA | ILE | B | 311 | 26.170 | 72.590 | 69.923 | 1.00 | 36.36 |
| 2472 | CB | ILE | B | 311 | 26.066 | 71.421 | 69.009 | 1.00 | 42.14 |
| 2473 | CG1 | ILE | B | 311 | 26.191 | 70.196 | 69.587 | 1.00 | 39.35 |
| 2474 | CD1 | ILE | B | 311 | 25.826 | 68.938 | 68.734 | 1.00 | 41.03 |
| 2475 | CG2 | ILE | B | 311 | 27.183 | 71.480 | 67.817 | 1.00 | 43.34 |
| 2476 | C | ILE | B | 311 | 25.828 | 73.940 | 69.187 | 1.00 | 37.43 |
| 2477 | O | ILE | B | 311 | 26.810 | 74.547 | 68.730 | 1.00 | 41.57 |
| 2478 | N | MET | B | 312 | 24.620 | 74.369 | 69.197 | 1.00 | 35.81 |
| 2479 | CA | MET | B | 312 | 24.241 | 75.538 | 68.627 | 1.00 | 48.06 |
| 2480 | CB | MET | B | 312 | 22.632 | 75.799 | 68.411 | 1.00 | 50.08 |
| 2481 | CG | MET | B | 312 | 22.017 | 74.785 | 67.347 | 1.00 | 40.55 |
| 2482 | SD | MET | B | 312 | 20.256 | 75.219 | 67.641 | 1.00 | 56.76 |
| 2483 | CE | MET | B | 312 | 19.709 | 73.976 | 68.035 | 1.00 | 60.29 |
| 2484 | C | MET | B | 312 | 24.596 | 76.870 | 69.414 | 1.00 | 50.66 |
| 2485 | O | MET | B | 312 | 24.526 | 77.956 | 68.867 | 1.00 | 45.83 |
| 2486 | N | LEU | B | 313 | 24.690 | 76.736 | 70.718 | 1.00 | 44.93 |
| 2487 | CA | LEU | B | 313 | 25.178 | 77.734 | 71.666 | 1.00 | 41.55 |
| 2488 | CB | LEU | B | 313 | 24.975 | 77.413 | 73.033 | 1.00 | 29.22 |
| 2489 | CG | LEU | B | 313 | 23.619 | 77.798 | 73.316 | 1.00 | 32.64 |
| 2490 | CD1 | LEU | B | 313 | 22.989 | 77.020 | 74.564 | 1.00 | 39.23 |
| 2491 | CD2 | LEU | B | 313 | 23.443 | 79.305 | 73.363 | 1.00 | 30.06 |
| 2492 | C | LEU | B | 313 | 26.632 | 77.770 | 71.384 | 1.00 | 41.42 |
| 2493 | O | LEU | B | 313 | 27.115 | 78.864 | 71.203 | 1.00 | 39.31 |
| 2494 | N | LEU | B | 314 | 27.252 | 76.599 | 71.047 | 1.00 | 41.28 |
| 2495 | CA | LEU | B | 314 | 28.694 | 76.475 | 70.777 | 1.00 | 40.18 |
| 2496 | CB | LEU | B | 314 | 29.168 | 75.185 | 70.928 | 1.00 | 37.72 |
| 2497 | CG | LEU | B | 314 | 29.743 | 74.653 | 72.251 | 1.00 | 45.82 |
| 2498 | CD1 | LEU | B | 314 | 30.190 | 73.124 | 72.172 | 1.00 | 36.99 |
| 2499 | CD2 | LEU | B | 314 | 31.042 | 75.397 | 72.663 | 1.00 | 43.47 |
| 2500 | C | LEU | B | 314 | 29.115 | 77.121 | 69.392 | 1.00 | 54.06 |
| 2501 | O | LEU | B | 314 | 30.241 | 77.751 | 69.180 | 1.00 | 49.68 |
| 2502 | N | GLU | B | 315 | 28.345 | 76.790 | 68.346 | 1.00 | 60.26 |
| 2503 | CA | GLU | B | 315 | 28.711 | 77.225 | 66.958 | 1.00 | 57.22 |
| 2504 | CB | GLU | B | 315 | 27.789 | 76.349 | 65.926 | 1.00 | 54.82 |
| 2505 | CG | GLU | B | 315 | 28.473 | 74.937 | 65.578 | 1.00 | 47.55 |
| 2506 | CD | GLU | B | 315 | 29.959 | 75.061 | 65.018 | 1.00 | 66.56 |
| 2507 | OE1 | GLU | B | 315 | 30.378 | 76.107 | 64.386 | 1.00 | 71.34 |
| 2508 | OE2 | GLU | B | 315 | 30.873 | 74.212 | 65.250 | 1.00 | 66.80 |
| 2509 | C | GLU | B | 315 | 28.218 | 78.738 | 66.962 | 1.00 | 57.50 |
| 2510 | O | GLU | B | 315 | 28.795 | 79.532 | 66.191 | 1.00 | 59.15 |
| 2511 | N | THR | B | 316 | 27.217 | 79.105 | 67.678 | 1.00 | 52.78 |
| 2512 | CA | THR | B | 316 | 26.838 | 80.531 | 67.733 | 1.00 | 51.20 |
| 2513 | CB | THR | B | 316 | 25.834 | 80.898 | 68.717 | 1.00 | 57.50 |
| 2514 | OG1 | THR | B | 316 | 24.441 | 80.632 | 68.221 | 1.00 | 64.56 |
| 2515 | CG2 | THR | B | 316 | 25.756 | 82.238 | 69.198 | 1.00 | 52.91 |
| 2516 | C | THR | B | 316 | 27.989 | 81.288 | 68.430 | 1.00 | 57.95 |
| 2517 | O | THR | B | 316 | 28.623 | 82.231 | 67.837 | 1.00 | 61.85 |
| 2518 | N | ALA | B | 317 | 28.578 | 80.706 | 69.411 | 1.00 | 57.22 |
| 2519 | CA | ALA | B | 317 | 29.641 | 81.279 | 70.058 | 1.00 | 63.28 |
| 2520 | CB | ALA | B | 317 | 30.121 | 80.592 | 71.332 | 1.00 | 55.94 |
| 2521 | C | ALA | B | 317 | 30.798 | 81.416 | 69.076 | 1.00 | 62.31 |
| 2522 | O | ALA | B | 317 | 31.564 | 82.528 | 69.007 | 1.00 | 67.15 |
| 2523 | N | ARG | B | 318 | 31.047 | 80.390 | 68.426 | 1.00 | 61.09 |
| 2524 | CA | ARG | B | 318 | 32.295 | 80.537 | 67.603 | 1.00 | 63.74 |
| 2525 | CB | ARG | B | 318 | 32.730 | 79.203 | 67.163 | 1.00 | 64.66 |
| 2526 | CG | ARG | B | 318 | 32.441 | 78.894 | 65.959 | 1.00 | 66.74 |
| 2527 | CD | ARG | B | 318 | 33.501 | 77.925 | 65.208 | 1.00 | 70.68 |
| 2528 | NE | ARG | B | 318 | 32.930 | 76.486 | 64.841 | 1.00 | 71.26 |
| 2529 | CZ | ARG | B | 318 | 33.768 | 75.499 | 64.497 | 1.00 | 67.45 |
| 2530 | NH1 | ARG | B | 318 | 34.890 | 75.807 | 64.276 | 1.00 | 67.59 |
| 2531 | NH2 | ARG | B | 318 | 33.505 | 74.292 | 64.199 | 1.00 | 77.12 |
| 2532 | C | ARG | B | 318 | 32.073 | 81.428 | 66.369 | 1.00 | 64.55 |
| 2533 | O | ARG | B | 318 | 33.008 | 81.604 | 65.611 | 1.00 | 63.09 |
| 2534 | N | ARG | B | 319 | 30.860 | 82.095 | 66.351 | 1.00 | 59.57 |
| 2535 | CA | ARG | B | 319 | 30.765 | 82.999 | 65.160 | 1.00 | 68.49 |
| 2536 | CB | ARG | B | 319 | 29.473 | 82.521 | 64.326 | 1.00 | 61.30 |
| 2537 | CG | ARG | B | 319 | 30.060 | 81.853 | 63.067 | 1.00 | 69.41 |
| 2538 | CD | ARG | B | 319 | 29.235 | 80.581 | 62.763 | 1.00 | 73.24 |
| 2539 | NE | ARG | B | 319 | 30.175 | 79.647 | 62.098 | 1.00 | 75.10 |
| 2540 | CZ | ARG | B | 319 | 29.984 | 78.369 | 62.112 | 1.00 | 79.76 |
| 2541 | NH1 | ARG | B | 319 | 28.933 | 77.857 | 62.748 | 1.00 | 73.63 |
| 2542 | NH2 | ARG | B | 319 | 30.844 | 77.583 | 61.539 | 1.00 | 68.15 |
| 2543 | C | ARG | B | 319 | 30.514 | 84.508 | 65.637 | 1.00 | 65.44 |
| 2544 | O | ARG | B | 319 | 29.829 | 85.398 | 64.966 | 1.00 | 61.07 |
| 2545 | N | TYR | B | 320 | 30.987 | 84.701 | 66.895 | 1.00 | 63.21 |
| 2546 | CA | TYR | B | 320 | 30.819 | 85.999 | 67.487 | 1.00 | 61.59 |
| 2547 | CB | TYR | B | 320 | 30.775 | 85.901 | 68.977 | 1.00 | 58.07 |
| 2548 | CG | TYR | B | 320 | 30.812 | 87.230 | 69.711 | 1.00 | 53.02 |
| 2549 | CD1 | TYR | B | 320 | 31.930 | 87.725 | 70.264 | 1.00 | 54.55 |
| 2550 | CE1 | TYR | B | 320 | 31.836 | 88.964 | 70.950 | 1.00 | 53.67 |
| 2551 | CZ | TYR | B | 320 | 30.717 | 89.582 | 71.153 | 1.00 | 53.87 |
| 2552 | OH | TYR | B | 320 | 30.609 | 90.785 | 71.918 | 1.00 | 54.97 |
| 2553 | CE2 | TYR | B | 320 | 29.522 | 88.994 | 70.707 | 1.00 | 56.50 |
| 2554 | CD2 | TYR | B | 320 | 29.645 | 87.868 | 69.986 | 1.00 | 58.99 |
| 2555 | C | TYR | B | 320 | 31.899 | 86.832 | 67.011 | 1.00 | 55.23 |
| 2556 | O | TYR | B | 320 | 32.878 | 86.389 | 66.887 | 1.00 | 58.77 |
| 2557 | N | ASN | B | 321 | 31.580 | 88.000 | 66.684 | 1.00 | 65.16 |
| 2558 | CA | ASN | B | 321 | 32.583 | 89.035 | 66.203 | 1.00 | 67.00 |
| 2559 | CB | ASN | B | 321 | 31.783 | 89.909 | 65.073 | 1.00 | 65.00 |
| 2560 | CG | ASN | B | 321 | 32.836 | 90.583 | 64.077 | 1.00 | 68.86 |
| 2561 | OD1 | ASN | B | 321 | 32.218 | 91.221 | 63.132 | 1.00 | 76.03 |
| 2562 | ND2 | ASN | B | 321 | 34.315 | 90.514 | 64.302 | 1.00 | 56.08 |
| 2563 | C | ASN | B | 321 | 32.879 | 89.996 | 67.279 | 1.00 | 60.46 |
| 2564 | O | ASN | B | 321 | 31.942 | 90.604 | 67.867 | 1.00 | 65.67 |
| 2565 | N | HIS | B | 322 | 34.074 | 90.206 | 67.470 | 1.00 | 68.48 |
| 2566 | CA | HIS | B | 322 | 34.486 | 91.228 | 68.587 | 1.00 | 73.34 |
| 2567 | CB | HIS | B | 322 | 35.894 | 90.991 | 69.233 | 1.00 | 71.05 |
| 2568 | CG | HIS | B | 322 | 35.890 | 91.351 | 70.731 | 1.00 | 82.08 |
| 2569 | ND1 | HIS | B | 322 | 34.747 | 91.147 | 71.589 | 1.00 | 87.14 |
| 2570 | CE1 | HIS | B | 322 | 35.017 | 91.506 | 72.853 | 1.00 | 73.65 |
| 2571 | NE2 | HIS | B | 322 | 36.326 | 91.869 | 72.867 | 1.00 | 80.65 |
| 2572 | CD2 | HIS | B | 322 | 36.862 | 91.841 | 71.558 | 1.00 | 72.77 |
| 2573 | C | HIS | B | 322 | 34.164 | 92.768 | 68.375 | 1.00 | 66.40 |
| 2574 | O | HIS | B | 322 | 33.837 | 93.373 | 69.334 | 1.00 | 67.21 |
| 2575 | N | GLU | B | 323 | 34.174 | 93.291 | 67.174 | 1.00 | 71.21 |
| 2576 | CA | GLU | B | 323 | 33.707 | 94.564 | 67.212 | 1.00 | 77.68 |
| 2577 | CB | GLU | B | 323 | 34.541 | 95.721 | 66.906 | 1.00 | 80.99 |
| 2578 | CG | GLU | B | 323 | 34.254 | 96.831 | 68.101 | 1.00 | 81.52 |
| 2579 | CD | GLU | B | 323 | 34.486 | 98.300 | 67.726 | 1.00 | 90.73 |
| 2580 | OE1 | GLU | B | 323 | 35.475 | 98.510 | 66.917 | 1.00 | 102.87 |
| 2581 | OE2 | GLU | B | 323 | 33.744 | 99.246 | 68.246 | 1.00 | 91.80 |
| 2582 | C | GLU | B | 323 | 32.281 | 94.831 | 66.835 | 1.00 | 79.43 |
| 2583 | O | GLU | B | 323 | 31.626 | 95.785 | 67.535 | 1.00 | 80.47 |
| 2584 | N | THR | B | 324 | 31.741 | 93.954 | 65.982 | 1.00 | 72.14 |
| 2585 | CA | THR | B | 324 | 30.345 | 94.187 | 65.612 | 1.00 | 69.64 |
| 2586 | CB | THR | B | 324 | 29.930 | 93.339 | 64.552 | 1.00 | 67.93 |
| 2587 | OG1 | THR | B | 324 | 30.970 | 92.349 | 64.445 | 1.00 | 79.89 |
| 2588 | CG2 | THR | B | 324 | 30.008 | 94.074 | 63.113 | 1.00 | 72.90 |
| 2589 | C | THR | B | 324 | 29.613 | 93.736 | 66.704 | 1.00 | 71.35 |
| 2590 | O | THR | B | 324 | 28.393 | 93.991 | 66.829 | 1.00 | 70.48 |
| 2591 | N | GLU | B | 325 | 30.336 | 92.943 | 67.540 | 1.00 | 73.92 |
| 2592 | CA | GLU | B | 325 | 29.628 | 92.339 | 68.693 | 1.00 | 75.54 |
| 2593 | CB | GLU | B | 325 | 29.186 | 93.383 | 69.839 | 1.00 | 71.78 |
| 2594 | CG | GLU | B | 325 | 30.258 | 94.467 | 69.915 | 1.00 | 78.66 |
| 2595 | CD | GLU | B | 325 | 30.335 | 95.091 | 71.351 | 1.00 | 77.65 |
| 2596 | OE1 | GLU | B | 325 | 29.404 | 95.606 | 71.855 | 1.00 | 79.58 |
| 2597 | OE2 | GLU | B | 325 | 31.348 | 94.845 | 72.040 | 1.00 | 82.11 |
| 2598 | C | GLU | B | 325 | 28.260 | 91.603 | 68.082 | 1.00 | 70.73 |
| 2599 | O | GLU | B | 325 | 27.097 | 91.742 | 68.635 | 1.00 | 66.33 |
| 2600 | N | CYS | B | 326 | 28.494 | 91.022 | 66.965 | 1.00 | 64.52 |
| 2601 | CA | CYS | B | 326 | 27.337 | 90.359 | 66.312 | 1.00 | 67.07 |
| 2602 | CB | CYS | B | 326 | 26.830 | 91.209 | 64.945 | 1.00 | 74.74 |
| 2603 | SG | CYS | B | 326 | 25.854 | 92.706 | 65.368 | 1.00 | 68.93 |
| 2604 | C | CYS | B | 326 | 27.664 | 88.903 | 65.914 | 1.00 | 62.47 |
| 2605 | O | CYS | B | 326 | 28.803 | 88.540 | 65.629 | 1.00 | 61.32 |
| 2606 | N | ILE | B | 327 | 26.596 | 88.125 | 65.896 | 1.00 | 62.77 |
| 2607 | CA | ILE | B | 327 | 26.825 | 86.679 | 65.506 | 1.00 | 65.69 |
| 2608 | CB | ILE | B | 327 | 25.734 | 85.833 | 66.108 | 1.00 | 62.81 |
| 2609 | CG1 | ILE | B | 327 | 25.699 | 85.979 | 67.621 | 1.00 | 57.04 |
| 2610 | CD1 | ILE | B | 327 | 27.160 | 85.753 | 68.002 | 1.00 | 57.90 |
| 2611 | CG2 | ILE | B | 327 | 25.925 | 84.543 | 65.811 | 1.00 | 56.79 |
| 2612 | C | ILE | B | 327 | 26.814 | 86.560 | 63.980 | 1.00 | 70.85 |
| 2613 | O | ILE | B | 327 | 25.808 | 87.044 | 63.339 | 1.00 | 68.43 |
| 2614 | N | THR | B | 328 | 27.826 | 85.849 | 63.444 | 1.00 | 74.31 |
| 2615 | CA | THR | B | 328 | 27.862 | 85.617 | 62.027 | 1.00 | 77.88 |
| 2616 | CB | THR | B | 328 | 29.079 | 85.878 | 61.499 | 1.00 | 76.18 |
| 2617 | OG1 | THR | B | 328 | 29.054 | 86.699 | 60.193 | 1.00 | 87.07 |
| 2618 | CG2 | THR | B | 328 | 29.651 | 84.611 | 61.306 | 1.00 | 69.42 |
| 2619 | C | THR | B | 328 | 27.538 | 84.230 | 61.518 | 1.00 | 86.32 |
| 2620 | O | THR | B | 328 | 27.848 | 83.224 | 62.152 | 1.00 | 88.10 |
| 2621 | N | PHE | B | 329 | 26.775 | 84.166 | 60.411 | 1.00 | 91.72 |
| 2622 | CA | PHE | B | 329 | 26.452 | 82.823 | 59.890 | 1.00 | 96.31 |
| 2623 | CB | PHE | B | 329 | 24.995 | 82.831 | 59.432 | 1.00 | 96.03 |
| 2624 | CG | PHE | B | 329 | 24.811 | 83.479 | 58.144 | 1.00 | 96.98 |
| 2625 | CD1 | PHE | B | 329 | 24.808 | 82.750 | 56.887 | 1.00 | 108.83 |
| 2626 | CE1 | PHE | B | 329 | 24.702 | 83.429 | 55.570 | 1.00 | 111.72 |
| 2627 | CZ | PHE | B | 329 | 24.650 | 84.895 | 55.544 | 1.00 | 114.57 |
| 2628 | CE2 | PHE | B | 329 | 24.715 | 85.558 | 56.851 | 1.00 | 113.77 |
| 2629 | CD2 | PHE | B | 329 | 24.787 | 84.778 | 58.122 | 1.00 | 98.68 |
| 2630 | C | PHE | B | 329 | 27.463 | 82.596 | 58.771 | 1.00 | 97.95 |
| 2631 | O | PHE | B | 329 | 28.091 | 81.513 | 58.593 | 1.00 | 102.22 |
| 2632 | N | LEU | B | 330 | 27.801 | 83.692 | 58.122 | 1.00 | 99.46 |
| 2633 | CA | LEU | B | 330 | 28.763 | 83.609 | 57.048 | 1.00 | 99.12 |
| 2634 | CB | LEU | B | 330 | 28.448 | 82.496 | 56.112 | 1.00 | 98.10 |
| 2635 | CG | LEU | B | 330 | 29.481 | 82.278 | 54.984 | 1.00 | 102.41 |
| 2636 | CD1 | LEU | B | 330 | 31.027 | 82.287 | 55.382 | 1.00 | 100.19 |
| 2637 | CD2 | LEU | B | 330 | 29.081 | 80.857 | 54.343 | 1.00 | 112.83 |
| 2638 | C | LEU | B | 330 | 28.849 | 84.952 | 56.309 | 1.00 | 99.47 |
| 2639 | O | LEU | B | 330 | 29.974 | 85.384 | 56.049 | 1.00 | 103.83 |
| 2640 | N | LYS | B | 331 | 27.744 | 85.613 | 55.952 | 1.00 | 96.96 |
| 2641 | CA | LYS | B | 331 | 27.831 | 86.934 | 55.298 | 1.00 | 98.37 |
| 2642 | CB | LYS | B | 331 | 27.268 | 86.980 | 53.911 | 1.00 | 102.58 |
| 2643 | CG | LYS | B | 331 | 28.113 | 86.052 | 52.923 | 1.00 | 107.93 |
| 2644 | CD | LYS | B | 331 | 27.036 | 85.406 | 51.700 | 1.00 | 105.27 |
| 2645 | CE | LYS | B | 331 | 27.842 | 84.412 | 50.674 | 1.00 | 106.60 |
| 2646 | NZ | LYS | B | 331 | 27.126 | 83.719 | 49.532 | 1.00 | 87.77 |
| 2647 | C | LYS | B | 331 | 27.040 | 87.899 | 55.991 | 1.00 | 97.46 |
| 2648 | O | LYS | B | 331 | 26.720 | 87.723 | 57.162 | 1.00 | 100.77 |
| 2649 | N | ASP | B | 332 | 26.673 | 88.950 | 55.315 | 1.00 | 95.42 |
| 2650 | CA | ASP | B | 332 | 25.895 | 89.988 | 55.948 | 1.00 | 95.39 |
| 2651 | CB | ASP | B | 332 | 25.240 | 90.915 | 54.941 | 1.00 | 99.84 |
| 2652 | CG | ASP | B | 332 | 26.248 | 92.060 | 54.549 | 1.00 | 106.41 |
| 2653 | OD1 | ASP | B | 332 | 26.148 | 92.556 | 53.387 | 1.00 | 115.62 |
| 2654 | OD2 | ASP | B | 332 | 27.234 | 92.504 | 55.365 | 1.00 | 109.27 |
| 2655 | C | ASP | B | 332 | 24.930 | 89.807 | 57.002 | 1.00 | 93.78 |
| 2656 | O | ASP | B | 332 | 24.788 | 90.634 | 57.997 | 1.00 | 93.30 |
| 2657 | N | PHE | B | 333 | 24.205 | 88.751 | 56.815 | 1.00 | 92.59 |
| 2658 | CA | PHE | B | 333 | 23.288 | 88.392 | 57.908 | 1.00 | 93.01 |
| 2659 | CB | PHE | B | 333 | 22.366 | 87.298 | 57.381 | 1.00 | 93.27 |
| 2660 | CG | PHE | B | 333 | 21.276 | 87.850 | 56.476 | 1.00 | 100.71 |
| 2661 | CD1 | PHE | B | 333 | 21.676 | 88.659 | 55.313 | 1.00 | 104.96 |
| 2662 | CE1 | PHE | B | 333 | 20.671 | 89.353 | 54.480 | 1.00 | 103.05 |
| 2663 | CZ | PHE | B | 333 | 19.148 | 89.224 | 54.829 | 1.00 | 107.06 |
| 2664 | CE2 | PHE | B | 333 | 18.761 | 88.347 | 56.090 | 1.00 | 110.04 |
| 2665 | CD2 | PHE | B | 333 | 19.842 | 87.733 | 56.870 | 1.00 | 102.86 |
| 2666 | C | PHE | B | 333 | 23.996 | 88.040 | 59.385 | 1.00 | 90.88 |
| 2667 | O | PHE | B | 333 | 24.298 | 86.882 | 59.697 | 1.00 | 90.25 |
| 2668 | N | THR | B | 334 | 24.154 | 89.078 | 60.185 | 1.00 | 84.40 |
| 2669 | CA | THR | B | 334 | 24.695 | 89.099 | 61.408 | 1.00 | 83.07 |
| 2670 | CB | THR | B | 334 | 26.005 | 89.869 | 61.294 | 1.00 | 83.97 |
| 2671 | OG1 | THR | B | 334 | 26.424 | 89.498 | 60.004 | 1.00 | 83.93 |
| 2672 | CG2 | THR | B | 334 | 27.211 | 89.293 | 62.280 | 1.00 | 86.33 |
| 2673 | C | THR | B | 334 | 23.728 | 89.756 | 62.294 | 1.00 | 82.45 |
| 2674 | O | THR | B | 334 | 22.728 | 90.575 | 61.957 | 1.00 | 80.83 |
| 2675 | N | TYR | B | 335 | 23.975 | 89.349 | 63.551 | 1.00 | 75.81 |
| 2676 | CA | TYR | B | 335 | 22.942 | 89.947 | 64.572 | 1.00 | 75.00 |
| 2677 | CB | TYR | B | 335 | 21.682 | 89.016 | 64.794 | 1.00 | 74.93 |
| 2678 | CG | TYR | B | 335 | 21.364 | 88.123 | 63.677 | 1.00 | 78.20 |
| 2679 | CD1 | TYR | B | 335 | 22.187 | 87.145 | 63.030 | 1.00 | 77.22 |
| 2680 | CE1 | TYR | B | 335 | 21.444 | 86.449 | 61.924 | 1.00 | 77.92 |
| 2681 | CZ | TYR | B | 335 | 20.199 | 86.781 | 61.452 | 1.00 | 70.57 |
| 2682 | OH | TYR | B | 335 | 19.742 | 86.051 | 60.284 | 1.00 | 87.36 |
| 2683 | CE2 | TYR | B | 335 | 19.531 | 87.679 | 62.108 | 1.00 | 89.00 |
| 2684 | CD2 | TYR | B | 335 | 20.086 | 88.329 | 63.181 | 1.00 | 91.31 |
| 2685 | C | TYR | B | 335 | 23.422 | 90.459 | 65.896 | 1.00 | 69.32 |
| 2686 | O | TYR | B | 335 | 24.454 | 90.253 | 66.261 | 1.00 | 72.11 |
| 2687 | N | SER | B | 336 | 22.610 | 91.199 | 66.547 | 1.00 | 68.57 |
| 2688 | CA | SER | B | 336 | 22.786 | 91.985 | 67.682 | 1.00 | 62.50 |
| 2689 | CB | SER | B | 336 | 22.545 | 93.468 | 67.237 | 1.00 | 62.77 |
| 2690 | OG | SER | B | 336 | 21.204 | 93.805 | 67.256 | 1.00 | 54.34 |
| 2691 | C | SER | B | 336 | 21.839 | 91.719 | 68.722 | 1.00 | 56.95 |
| 2692 | O | SER | B | 336 | 20.837 | 91.110 | 68.474 | 1.00 | 55.57 |
| 2693 | N | LYS | B | 337 | 22.245 | 91.980 | 69.906 | 1.00 | 59.06 |
| 2694 | CA | LYS | B | 337 | 21.401 | 91.672 | 71.082 | 1.00 | 65.18 |
| 2695 | CB | LYS | B | 337 | 21.981 | 92.085 | 72.472 | 1.00 | 62.25 |
| 2696 | CG | LYS | B | 337 | 23.158 | 91.183 | 72.975 | 1.00 | 75.18 |
| 2697 | CD | LYS | B | 337 | 24.128 | 91.873 | 74.164 | 1.00 | 69.42 |
| 2698 | CE | LYS | B | 337 | 23.258 | 92.166 | 75.429 | 1.00 | 82.02 |
| 2699 | NZ | LYS | B | 337 | 22.988 | 93.840 | 75.729 | 1.00 | 88.14 |
| 2700 | C | LYS | B | 337 | 19.898 | 92.123 | 70.979 | 1.00 | 65.87 |
| 2701 | O | LYS | B | 337 | 18.790 | 91.514 | 71.305 | 1.00 | 62.80 |
| 2702 | N | ASP | B | 338 | 19.841 | 93.263 | 70.436 | 1.00 | 68.89 |
| 2703 | CA | ASP | B | 338 | 18.509 | 93.856 | 70.128 | 1.00 | 66.22 |
| 2704 | CB | ASP | B | 338 | 18.759 | 95.420 | 69.801 | 1.00 | 67.25 |
| 2705 | CG | ASP | B | 338 | 19.017 | 96.214 | 71.180 | 1.00 | 78.76 |
| 2706 | OD1 | ASP | B | 338 | 18.059 | 96.369 | 72.063 | 1.00 | 80.15 |
| 2707 | OD2 | ASP | B | 338 | 20.106 | 96.727 | 71.473 | 1.00 | 82.82 |
| 2708 | C | ASP | B | 338 | 17.752 | 93.074 | 69.013 | 1.00 | 63.09 |
| 2709 | O | ASP | B | 338 | 16.439 | 92.773 | 69.199 | 1.00 | 62.26 |
| 2710 | N | ASP | B | 339 | 18.472 | 92.767 | 67.957 | 1.00 | 53.26 |
| 2711 | CA | ASP | B | 339 | 17.730 | 92.045 | 67.051 | 1.00 | 60.52 |
| 2712 | CB | ASP | B | 339 | 18.725 | 91.352 | 65.998 | 1.00 | 61.80 |
| 2713 | CG | ASP | B | 339 | 19.365 | 92.313 | 65.200 | 1.00 | 63.07 |
| 2714 | OD1 | ASP | B | 339 | 18.679 | 93.307 | 64.790 | 1.00 | 70.88 |
| 2715 | OD2 | ASP | B | 339 | 20.437 | 92.232 | 64.854 | 1.00 | 67.08 |
| 2716 | C | ASP | B | 339 | 16.863 | 90.880 | 67.818 | 1.00 | 65.06 |
| 2717 | O | ASP | B | 339 | 15.506 | 90.720 | 67.561 | 1.00 | 61.45 |
| 2718 | N | PHE | B | 340 | 17.662 | 90.149 | 68.728 | 1.00 | 61.06 |
| 2719 | CA | PHE | B | 340 | 17.220 | 89.050 | 69.494 | 1.00 | 60.32 |
| 2720 | CB | PHE | B | 340 | 18.300 | 88.290 | 70.495 | 1.00 | 56.10 |
| 2721 | CG | PHE | B | 340 | 19.434 | 87.535 | 69.783 | 1.00 | 47.88 |
| 2722 | CD1 | PHE | B | 340 | 20.202 | 88.004 | 68.847 | 1.00 | 47.77 |
| 2723 | CE1 | PHE | B | 340 | 21.132 | 87.399 | 68.149 | 1.00 | 45.70 |
| 2724 | CZ | PHE | B | 340 | 21.320 | 85.899 | 68.401 | 1.00 | 55.25 |
| 2725 | CE2 | PHE | B | 340 | 20.409 | 85.312 | 69.255 | 1.00 | 49.14 |
| 2726 | CD2 | PHE | B | 340 | 19.482 | 86.220 | 69.988 | 1.00 | 55.69 |
| 2727 | C | PHE | B | 340 | 16.017 | 89.484 | 70.175 | 1.00 | 62.83 |
| 2728 | O | PHE | B | 340 | 14.955 | 88.699 | 70.110 | 1.00 | 67.32 |
| 2729 | N | HIS | B | 341 | 16.072 | 90.651 | 70.815 | 1.00 | 64.53 |
| 2730 | CA | HIS | B | 341 | 14.814 | 91.067 | 71.551 | 1.00 | 71.00 |
| 2731 | CB | HIS | B | 341 | 15.007 | 92.393 | 72.368 | 1.00 | 72.94 |
| 2732 | CG | HIS | B | 341 | 16.122 | 92.302 | 73.297 | 1.00 | 69.49 |
| 2733 | ND1 | HIS | B | 341 | 17.464 | 92.315 | 72.856 | 1.00 | 73.08 |
| 2734 | CE1 | HIS | B | 341 | 18.264 | 92.173 | 73.954 | 1.00 | 78.41 |
| 2735 | NE2 | HIS | B | 341 | 17.479 | 91.902 | 75.023 | 1.00 | 82.09 |
| 2736 | CD2 | HIS | B | 341 | 16.126 | 91.990 | 74.616 | 1.00 | 77.34 |
| 2737 | C | HIS | B | 341 | 13.616 | 91.295 | 70.652 | 1.00 | 66.90 |
| 2738 | O | HIS | B | 341 | 12.510 | 90.986 | 71.057 | 1.00 | 60.48 |
| 2739 | N | ARG | B | 342 | 14.012 | 91.759 | 69.483 | 1.00 | 67.75 |
| 2740 | CA | ARG | B | 342 | 13.037 | 91.936 | 68.350 | 1.00 | 73.18 |
| 2741 | CB | ARG | B | 342 | 13.829 | 92.432 | 67.008 | 1.00 | 74.27 |
| 2742 | CG | ARG | B | 342 | 13.585 | 94.024 | 66.904 | 1.00 | 61.57 |
| 2743 | CD | ARG | B | 342 | 14.575 | 94.742 | 66.346 | 1.00 | 60.29 |
| 2744 | NE | ARG | B | 342 | 14.705 | 96.162 | 66.846 | 1.00 | 71.95 |
| 2745 | CZ | ARG | B | 342 | 15.904 | 96.835 | 67.255 | 1.00 | 67.30 |
| 2746 | NH1 | ARG | B | 342 | 17.125 | 96.343 | 66.996 | 1.00 | 70.43 |
| 2747 | NH2 | ARG | B | 342 | 15.916 | 98.007 | 67.794 | 1.00 | 68.99 |
| 2748 | C | ARG | B | 342 | 12.209 | 90.669 | 67.936 | 1.00 | 70.00 |
| 2749 | O | ARG | B | 342 | 10.877 | 90.653 | 67.865 | 1.00 | 68.24 |
| 2750 | N | ALA | B | 343 | 12.938 | 89.619 | 67.870 | 1.00 | 65.37 |
| 2751 | CA | ALA | B | 343 | 12.336 | 88.282 | 67.551 | 1.00 | 57.52 |
| 2752 | CB | ALA | B | 343 | 13.328 | 87.443 | 66.900 | 1.00 | 52.06 |
| 2753 | C | ALA | B | 343 | 11.623 | 87.817 | 68.626 | 1.00 | 60.04 |
| 2754 | O | ALA | B | 343 | 10.975 | 86.799 | 68.619 | 1.00 | 63.88 |
| 2755 | N | GLY | B | 344 | 11.558 | 88.588 | 69.693 | 1.00 | 65.13 |
| 2756 | CA | GLY | B | 344 | 10.708 | 88.116 | 70.932 | 1.00 | 63.20 |
| 2757 | C | GLY | B | 344 | 11.503 | 87.321 | 72.005 | 1.00 | 61.28 |
| 2758 | O | GLY | B | 344 | 11.032 | 86.873 | 73.056 | 1.00 | 65.97 |
| 2759 | N | LEU | B | 345 | 12.792 | 87.264 | 71.820 | 1.00 | 64.60 |
| 2760 | CA | LEU | B | 345 | 13.716 | 86.536 | 72.858 | 1.00 | 62.54 |
| 2761 | CB | LEU | B | 345 | 15.061 | 86.201 | 72.183 | 1.00 | 57.64 |
| 2762 | CG | LEU | B | 345 | 15.157 | 85.573 | 70.740 | 1.00 | 62.93 |
| 2763 | CD1 | LEU | B | 345 | 16.123 | 84.434 | 70.841 | 1.00 | 76.97 |
| 2764 | CD2 | LEU | B | 345 | 13.802 | 85.098 | 69.952 | 1.00 | 59.00 |
| 2765 | C | LEU | B | 345 | 13.874 | 87.246 | 73.974 | 1.00 | 63.15 |
| 2766 | O | LEU | B | 345 | 14.359 | 88.327 | 73.807 | 1.00 | 76.57 |
| 2767 | N | GLN | B | 346 | 13.552 | 86.790 | 75.143 | 1.00 | 59.15 |
| 2768 | CA | GLN | B | 346 | 13.725 | 87.487 | 76.318 | 1.00 | 64.37 |
| 2769 | CB | GLN | B | 346 | 12.880 | 86.738 | 77.398 | 1.00 | 63.91 |
| 2770 | CG | GLN | B | 346 | 13.290 | 85.212 | 77.500 | 1.00 | 73.12 |
| 2771 | CD | GLN | B | 346 | 12.419 | 84.464 | 78.569 | 1.00 | 71.74 |
| 2772 | OE1 | GLN | B | 346 | 12.641 | 83.369 | 78.841 | 1.00 | 63.96 |
| 2773 | NE2 | GLN | B | 346 | 11.299 | 85.087 | 79.033 | 1.00 | 70.06 |
| 2774 | C | GLN | B | 346 | 15.261 | 87.723 | 76.891 | 1.00 | 65.83 |
| 2775 | O | GLN | B | 346 | 16.065 | 86.955 | 76.681 | 1.00 | 70.72 |
| 2776 | N | VAL | B | 347 | 15.586 | 88.724 | 77.526 | 1.00 | 59.97 |
| 2777 | CA | VAL | B | 347 | 16.843 | 89.122 | 77.777 | 1.00 | 58.82 |
| 2778 | CB | VAL | B | 347 | 16.779 | 90.406 | 78.635 | 1.00 | 59.13 |
| 2779 | CG1 | VAL | B | 347 | 15.764 | 90.275 | 80.040 | 1.00 | 60.73 |
| 2780 | CG2 | VAL | B | 347 | 18.034 | 90.918 | 79.113 | 1.00 | 67.69 |
| 2781 | C | VAL | B | 347 | 17.480 | 88.087 | 78.667 | 1.00 | 64.04 |
| 2782 | O | VAL | B | 347 | 18.669 | 88.031 | 78.690 | 1.00 | 62.73 |
| 2783 | N | GLU | B | 348 | 16.681 | 87.362 | 79.495 | 1.00 | 61.58 |
| 2784 | CA | GLU | B | 348 | 17.082 | 86.396 | 80.301 | 1.00 | 56.15 |
| 2785 | CB | GLU | B | 348 | 15.957 | 85.918 | 81.173 | 1.00 | 61.69 |
| 2786 | CG | GLU | B | 348 | 14.512 | 86.339 | 80.411 | 1.00 | 73.26 |
| 2787 | CD | GLU | B | 348 | 13.773 | 87.410 | 81.118 | 1.00 | 67.05 |
| 2788 | OE1 | GLU | B | 348 | 12.760 | 87.178 | 81.913 | 1.00 | 70.33 |
| 2789 | OE2 | GLU | B | 348 | 14.273 | 88.437 | 80.948 | 1.00 | 69.87 |
| 2790 | C | GLU | B | 348 | 17.671 | 85.224 | 79.631 | 1.00 | 57.15 |
| 2791 | O | GLU | B | 348 | 18.327 | 84.398 | 80.339 | 1.00 | 59.19 |
| 2792 | N | PHE | B | 349 | 17.792 | 85.189 | 78.303 | 1.00 | 55.88 |
| 2793 | CA | PHE | B | 349 | 18.444 | 84.324 | 77.568 | 1.00 | 46.69 |
| 2794 | CB | PHE | B | 349 | 17.345 | 83.467 | 76.750 | 1.00 | 54.67 |
| 2795 | CG | PHE | B | 349 | 17.898 | 82.462 | 75.751 | 1.00 | 56.05 |
| 2796 | CD1 | PHE | B | 349 | 18.897 | 81.521 | 76.196 | 1.00 | 57.34 |
| 2797 | CE1 | PHE | B | 349 | 19.455 | 80.643 | 75.414 | 1.00 | 52.74 |
| 2798 | CZ | PHE | B | 349 | 19.003 | 80.373 | 74.054 | 1.00 | 53.17 |
| 2799 | CE2 | PHE | B | 349 | 18.060 | 81.181 | 73.542 | 1.00 | 47.95 |
| 2800 | CD2 | PHE | B | 349 | 17.487 | 82.349 | 74.549 | 1.00 | 59.50 |
| 2801 | C | PHE | B | 349 | 19.381 | 84.858 | 76.696 | 1.00 | 46.68 |
| 2802 | O | PHE | B | 349 | 20.508 | 84.295 | 76.491 | 1.00 | 49.18 |
| 2803 | N | ILE | B | 350 | 19.087 | 85.971 | 76.173 | 1.00 | 44.99 |
| 2804 | CA | ILE | B | 350 | 20.099 | 86.710 | 75.203 | 1.00 | 47.50 |
| 2805 | CB | ILE | B | 350 | 19.427 | 88.047 | 74.637 | 1.00 | 47.55 |
| 2806 | CG1 | ILE | B | 350 | 18.113 | 87.541 | 73.797 | 1.00 | 48.34 |
| 2807 | CD1 | ILE | B | 350 | 17.011 | 88.771 | 74.039 | 1.00 | 59.54 |
| 2808 | CG2 | ILE | B | 350 | 20.235 | 88.800 | 73.720 | 1.00 | 55.42 |
| 2809 | C | ILE | B | 350 | 21.452 | 87.192 | 75.913 | 1.00 | 44.46 |
| 2810 | O | ILE | B | 350 | 22.534 | 86.952 | 75.262 | 1.00 | 45.96 |
| 2811 | N | ASN | B | 351 | 21.327 | 87.665 | 77.121 | 1.00 | 40.71 |
| 2812 | CA | ASN | B | 351 | 22.644 | 88.215 | 77.719 | 1.00 | 47.42 |
| 2813 | CB | ASN | B | 351 | 22.251 | 88.814 | 79.045 | 1.00 | 45.36 |
| 2814 | CG | ASN | B | 351 | 21.514 | 90.145 | 78.898 | 1.00 | 50.17 |
| 2815 | OD1 | ASN | B | 351 | 21.495 | 90.817 | 77.832 | 1.00 | 51.44 |
| 2816 | ND2 | ASN | B | 351 | 20.830 | 90.523 | 80.022 | 1.00 | 50.37 |
| 2817 | C | ASN | B | 351 | 23.588 | 86.934 | 78.059 | 1.00 | 41.66 |
| 2818 | O | ASN | B | 351 | 24.725 | 86.884 | 77.587 | 1.00 | 39.55 |
| 2819 | N | PRO | B | 352 | 22.937 | 85.903 | 78.537 | 1.00 | 36.22 |
| 2820 | CA | PRO | B | 352 | 23.682 | 84.683 | 78.700 | 1.00 | 34.72 |
| 2821 | CB | PRO | B | 352 | 22.753 | 83.723 | 79.279 | 1.00 | 36.88 |
| 2822 | CG | PRO | B | 352 | 21.811 | 84.653 | 80.164 | 1.00 | 46.71 |
| 2823 | CD | PRO | B | 352 | 21.621 | 85.892 | 79.041 | 1.00 | 35.82 |
| 2824 | C | PRO | B | 352 | 24.237 | 84.224 | 77.530 | 1.00 | 36.30 |
| 2825 | O | PRO | B | 352 | 25.419 | 83.761 | 77.383 | 1.00 | 40.39 |
| 2826 | N | ILE | B | 353 | 23.474 | 84.391 | 76.372 | 1.00 | 41.10 |
| 2827 | CA | ILE | B | 353 | 24.122 | 83.951 | 75.074 | 1.00 | 39.30 |
| 2828 | CB | ILE | B | 353 | 23.105 | 83.987 | 74.027 | 1.00 | 42.13 |
| 2829 | CG1 | ILE | B | 353 | 21.891 | 82.903 | 74.260 | 1.00 | 43.94 |
| 2830 | CD1 | ILE | B | 353 | 20.748 | 82.937 | 73.139 | 1.00 | 35.70 |
| 2831 | CG2 | ILE | B | 353 | 23.816 | 83.728 | 72.610 | 1.00 | 32.18 |
| 2832 | C | ILE | B | 353 | 25.291 | 84.644 | 74.751 | 1.00 | 38.70 |
| 2833 | O | ILE | B | 353 | 26.320 | 84.176 | 74.244 | 1.00 | 38.63 |
| 2834 | N | PHE | B | 354 | 25.195 | 85.973 | 75.050 | 1.00 | 47.94 |
| 2835 | CA | PHE | B | 354 | 26.388 | 86.815 | 74.814 | 1.00 | 50.23 |
| 2836 | CB | PHE | B | 354 | 25.749 | 88.409 | 74.654 | 1.00 | 50.03 |
| 2837 | CG | PHE | B | 354 | 25.108 | 88.608 | 73.274 | 1.00 | 55.16 |
| 2838 | CD1 | PHE | B | 354 | 23.865 | 88.155 | 73.072 | 1.00 | 55.03 |
| 2839 | CE1 | PHE | B | 354 | 23.259 | 88.195 | 71.851 | 1.00 | 54.17 |
| 2840 | CZ | PHE | B | 354 | 23.881 | 88.570 | 70.739 | 1.00 | 56.66 |
| 2841 | CE2 | PHE | B | 354 | 25.226 | 89.026 | 70.978 | 1.00 | 68.88 |
| 2842 | CD2 | PHE | B | 354 | 25.851 | 88.951 | 72.206 | 1.00 | 45.25 |
| 2843 | C | PHE | B | 354 | 27.526 | 86.798 | 75.713 | 1.00 | 40.23 |
| 2844 | O | PHE | B | 354 | 28.733 | 86.925 | 75.321 | 1.00 | 43.29 |
| 2845 | N | GLU | B | 355 | 27.211 | 86.597 | 76.921 | 1.00 | 40.93 |
| 2846 | CA | GLU | B | 355 | 28.523 | 86.397 | 77.978 | 1.00 | 37.60 |
| 2847 | CB | GLU | B | 355 | 27.902 | 86.380 | 79.277 | 1.00 | 44.07 |
| 2848 | CG | GLU | B | 355 | 27.453 | 87.768 | 79.784 | 1.00 | 40.87 |
| 2849 | CD | GLU | B | 355 | 26.128 | 87.705 | 80.643 | 1.00 | 54.01 |
| 2850 | OE1 | GLU | B | 355 | 25.652 | 86.568 | 81.136 | 1.00 | 55.64 |
| 2851 | OE2 | GLU | B | 355 | 25.515 | 88.734 | 80.860 | 1.00 | 57.08 |
| 2852 | C | GLU | B | 355 | 29.098 | 85.076 | 77.698 | 1.00 | 35.92 |
| 2853 | O | GLU | B | 355 | 30.454 | 84.938 | 77.539 | 1.00 | 34.43 |
| 2854 | N | PHE | B | 356 | 28.248 | 84.085 | 77.222 | 1.00 | 36.19 |
| 2855 | CA | PHE | B | 356 | 28.898 | 82.751 | 76.709 | 1.00 | 36.57 |
| 2856 | CB | PHE | B | 356 | 27.756 | 81.632 | 76.594 | 1.00 | 35.87 |
| 2857 | CG | PHE | B | 356 | 28.340 | 80.354 | 76.177 | 1.00 | 35.72 |
| 2858 | CD1 | PHE | B | 356 | 29.015 | 79.569 | 77.101 | 1.00 | 33.72 |
| 2859 | CE1 | PHE | B | 356 | 29.544 | 78.291 | 76.771 | 1.00 | 39.96 |
| 2860 | CZ | PHE | B | 356 | 29.540 | 77.881 | 75.360 | 1.00 | 40.70 |
| 2861 | CE2 | PHE | B | 356 | 28.833 | 78.674 | 74.387 | 1.00 | 32.50 |
| 2862 | CD2 | PHE | B | 356 | 28.366 | 79.897 | 74.811 | 1.00 | 41.44 |
| 2863 | C | PHE | B | 356 | 29.852 | 82.882 | 75.662 | 1.00 | 33.57 |
| 2864 | O | PHE | B | 356 | 31.075 | 82.374 | 75.608 | 1.00 | 35.52 |
| 2865 | N | SER | B | 357 | 29.407 | 83.703 | 74.694 | 1.00 | 34.35 |
| 2866 | CA | SER | B | 357 | 30.297 | 83.917 | 73.570 | 1.00 | 33.77 |
| 2867 | CB | SER | B | 357 | 29.364 | 84.692 | 72.534 | 1.00 | 47.00 |
| 2868 | OG | SER | B | 357 | 27.969 | 84.212 | 72.666 | 1.00 | 41.44 |
| 2869 | C | SER | B | 357 | 31.489 | 84.718 | 73.839 | 1.00 | 31.31 |
| 2870 | O | SER | B | 357 | 32.573 | 84.504 | 73.262 | 1.00 | 32.90 |
| 2871 | N | ARG | B | 358 | 31.360 | 85.621 | 74.748 | 1.00 | 34.45 |
| 2872 | CA | ARG | B | 358 | 32.615 | 86.391 | 75.106 | 1.00 | 34.95 |
| 2873 | CB | ARG | B | 358 | 32.189 | 87.572 | 76.022 | 1.00 | 39.55 |
| 2874 | CG | ARG | B | 358 | 31.744 | 88.997 | 75.200 | 1.00 | 41.56 |
| 2875 | CD | ARG | B | 358 | 31.059 | 89.917 | 76.494 | 1.00 | 44.77 |
| 2876 | NE | ARG | B | 358 | 29.771 | 90.142 | 76.015 | 1.00 | 45.47 |
| 2877 | CZ | ARG | B | 358 | 28.789 | 90.137 | 76.850 | 1.00 | 49.55 |
| 2878 | NH1 | ARG | B | 358 | 28.973 | 89.970 | 78.162 | 1.00 | 42.75 |
| 2879 | NH2 | ARG | B | 358 | 27.510 | 90.492 | 76.442 | 1.00 | 56.37 |
| 2880 | C | ARG | B | 358 | 33.558 | 85.457 | 75.884 | 1.00 | 35.05 |
| 2881 | O | ARG | B | 358 | 34.790 | 85.462 | 75.700 | 1.00 | 35.35 |
| 2882 | N | ALA | B | 359 | 32.926 | 84.629 | 76.657 | 1.00 | 35.52 |
| 2883 | CA | ALA | B | 359 | 33.812 | 83.717 | 77.462 | 1.00 | 35.54 |
| 2884 | CB | ALA | B | 359 | 32.818 | 82.947 | 78.509 | 1.00 | 31.23 |
| 2885 | C | ALA | B | 359 | 34.462 | 82.680 | 76.523 | 1.00 | 37.97 |
| 2886 | O | ALA | B | 359 | 35.668 | 82.350 | 76.637 | 1.00 | 33.88 |
| 2887 | N | MET | B | 360 | 33.706 | 82.100 | 75.643 | 1.00 | 41.47 |
| 2888 | CA | MET | B | 360 | 34.449 | 81.111 | 74.707 | 1.00 | 36.12 |
| 2889 | CB | MET | B | 360 | 33.314 | 80.532 | 73.676 | 1.00 | 35.78 |
| 2890 | CG | MET | B | 360 | 32.295 | 79.532 | 74.412 | 1.00 | 36.49 |
| 2891 | SD | MET | B | 360 | 33.230 | 78.304 | 75.227 | 1.00 | 33.45 |
| 2892 | CE | MET | B | 360 | 33.781 | 77.421 | 73.620 | 1.00 | 44.23 |
| 2893 | C | MET | B | 360 | 35.405 | 81.879 | 74.052 | 1.00 | 38.81 |
| 2894 | O | MET | B | 360 | 36.534 | 81.346 | 73.856 | 1.00 | 38.37 |
| 2895 | N | ARG | B | 361 | 35.055 | 83.136 | 73.576 | 1.00 | 37.37 |
| 2896 | CA | ARG | B | 361 | 36.248 | 83.826 | 72.876 | 1.00 | 44.82 |
| 2897 | CB | ARG | B | 361 | 35.780 | 85.316 | 72.522 | 1.00 | 44.87 |
| 2898 | CG | ARG | B | 361 | 36.987 | 86.109 | 71.904 | 1.00 | 44.84 |
| 2899 | CD | ARG | B | 361 | 36.466 | 87.439 | 71.655 | 1.00 | 51.90 |
| 2900 | NE | ARG | B | 361 | 37.276 | 88.236 | 71.019 | 1.00 | 60.42 |
| 2901 | CZ | ARG | B | 361 | 38.134 | 89.185 | 71.515 | 1.00 | 68.73 |
| 2902 | NH1 | ARG | B | 361 | 38.077 | 89.389 | 72.838 | 1.00 | 60.60 |
| 2903 | NH2 | ARG | B | 361 | 38.846 | 90.055 | 70.621 | 1.00 | 78.26 |
| 2904 | C | ARG | B | 361 | 37.480 | 83.977 | 73.692 | 1.00 | 46.62 |
| 2905 | O | ARG | B | 361 | 38.727 | 83.585 | 73.269 | 1.00 | 49.98 |
| 2906 | N | ARG | B | 362 | 37.353 | 84.518 | 74.826 | 1.00 | 41.39 |
| 2907 | CA | ARG | B | 362 | 38.668 | 84.561 | 75.685 | 1.00 | 42.68 |
| 2908 | CB | ARG | B | 362 | 38.178 | 85.012 | 77.142 | 1.00 | 42.44 |
| 2909 | CG | ARG | B | 362 | 37.619 | 86.567 | 77.136 | 1.00 | 42.82 |
| 2910 | CD | ARG | B | 362 | 37.308 | 86.945 | 78.575 | 1.00 | 38.22 |
| 2911 | NE | ARG | B | 362 | 35.868 | 87.079 | 78.788 | 1.00 | 41.73 |
| 2912 | CZ | ARG | B | 362 | 35.255 | 86.428 | 79.619 | 1.00 | 38.95 |
| 2913 | NH1 | ARG | B | 362 | 35.845 | 85.463 | 80.223 | 1.00 | 45.47 |
| 2914 | NH2 | ARG | B | 362 | 33.998 | 86.508 | 79.660 | 1.00 | 36.14 |
| 2915 | C | ARG | B | 362 | 39.358 | 83.319 | 75.688 | 1.00 | 40.83 |
| 2916 | O | ARG | B | 362 | 40.460 | 83.238 | 76.042 | 1.00 | 48.50 |
| 2917 | N | LEU | B | 363 | 38.700 | 82.197 | 75.579 | 1.00 | 38.38 |
| 2918 | CA | LEU | B | 363 | 39.437 | 80.922 | 75.664 | 1.00 | 39.93 |
| 2919 | CB | LEU | B | 363 | 38.359 | 79.771 | 75.741 | 1.00 | 40.71 |
| 2920 | CG | LEU | B | 363 | 37.949 | 79.299 | 77.014 | 1.00 | 38.90 |
| 2921 | CD1 | LEU | B | 363 | 36.937 | 78.240 | 76.961 | 1.00 | 33.87 |
| 2922 | CD2 | LEU | B | 363 | 39.129 | 78.816 | 77.709 | 1.00 | 42.27 |
| 2923 | C | LEU | B | 363 | 40.232 | 80.692 | 74.366 | 1.00 | 37.22 |
| 2924 | O | LEU | B | 363 | 41.222 | 79.915 | 74.404 | 1.00 | 34.01 |
| 2925 | N | GLY | B | 364 | 39.909 | 81.449 | 73.270 | 1.00 | 39.17 |
| 2926 | CA | GLY | B | 364 | 40.736 | 81.431 | 71.959 | 1.00 | 37.89 |
| 2927 | C | GLY | B | 364 | 40.810 | 79.959 | 71.399 | 1.00 | 44.21 |
| 2928 | O | GLY | B | 364 | 41.880 | 79.338 | 71.288 | 1.00 | 40.61 |
| 2929 | N | LEU | B | 365 | 39.637 | 79.369 | 71.190 | 1.00 | 43.34 |
| 2930 | CA | LEU | B | 365 | 39.694 | 77.882 | 70.801 | 1.00 | 49.01 |
| 2931 | CB | LEU | B | 365 | 38.272 | 77.164 | 71.229 | 1.00 | 42.87 |
| 2932 | CG | LEU | B | 365 | 38.161 | 77.232 | 72.788 | 1.00 | 49.95 |
| 2933 | CD1 | LEU | B | 365 | 36.743 | 76.607 | 72.908 | 1.00 | 58.47 |
| 2934 | CD2 | LEU | B | 365 | 39.116 | 76.275 | 73.403 | 1.00 | 50.07 |
| 2935 | C | LEU | B | 365 | 39.830 | 77.676 | 69.298 | 1.00 | 45.16 |
| 2936 | O | LEU | B | 365 | 39.156 | 78.429 | 68.524 | 1.00 | 45.24 |
| 2937 | N | ASP | B | 366 | 40.565 | 76.692 | 69.046 | 1.00 | 48.71 |
| 2938 | CA | ASP | B | 366 | 40.787 | 76.030 | 67.592 | 1.00 | 54.79 |
| 2939 | CB | ASP | B | 366 | 41.586 | 74.767 | 67.763 | 1.00 | 51.18 |
| 2940 | CG | ASP | B | 366 | 42.955 | 75.100 | 67.351 | 1.00 | 58.96 |
| 2941 | OD1 | ASP | B | 366 | 42.825 | 75.849 | 66.448 | 1.00 | 65.21 |
| 2942 | OD2 | ASP | B | 366 | 44.005 | 74.650 | 67.781 | 1.00 | 53.99 |
| 2943 | C | ASP | B | 366 | 39.659 | 75.446 | 67.061 | 1.00 | 59.19 |
| 2944 | O | ASP | B | 366 | 38.335 | 75.346 | 67.602 | 1.00 | 52.59 |
| 2945 | N | ASP | B | 367 | 40.020 | 74.916 | 65.925 | 1.00 | 60.01 |
| 2946 | CA | ASP | B | 367 | 38.976 | 74.117 | 65.057 | 1.00 | 53.72 |
| 2947 | CB | ASP | B | 367 | 39.632 | 74.063 | 63.588 | 1.00 | 56.58 |
| 2948 | CG | ASP | B | 367 | 39.019 | 75.126 | 62.645 | 1.00 | 69.39 |
| 2949 | OD1 | ASP | B | 367 | 38.272 | 76.070 | 63.065 | 1.00 | 68.05 |
| 2950 | OD2 | ASP | B | 367 | 39.339 | 75.032 | 61.444 | 1.00 | 78.09 |
| 2951 | C | ASP | B | 367 | 38.927 | 72.789 | 65.678 | 1.00 | 43.49 |
| 2952 | O | ASP | B | 367 | 37.896 | 72.221 | 65.734 | 1.00 | 49.56 |
| 2953 | N | ALA | B | 368 | 40.048 | 72.270 | 65.943 | 1.00 | 45.38 |
| 2954 | CA | ALA | B | 368 | 40.214 | 71.063 | 66.449 | 1.00 | 50.48 |
| 2955 | CB | ALA | B | 368 | 41.596 | 70.645 | 66.677 | 1.00 | 48.33 |
| 2956 | C | ALA | B | 368 | 39.304 | 70.977 | 67.802 | 1.00 | 52.64 |
| 2957 | O | ALA | B | 368 | 38.279 | 70.286 | 67.850 | 1.00 | 48.37 |
| 2958 | N | GLU | B | 369 | 39.807 | 71.840 | 68.718 | 1.00 | 45.67 |
| 2959 | CA | GLU | B | 369 | 39.101 | 72.143 | 70.017 | 1.00 | 47.42 |
| 2960 | CB | GLU | B | 369 | 39.774 | 73.382 | 70.752 | 1.00 | 39.41 |
| 2961 | CG | GLU | B | 369 | 41.226 | 73.138 | 70.884 | 1.00 | 47.88 |
| 2962 | CD | GLU | B | 369 | 42.177 | 74.344 | 71.359 | 1.00 | 50.37 |
| 2963 | OE1 | GLU | B | 369 | 41.953 | 75.426 | 70.859 | 1.00 | 49.12 |
| 2964 | OE2 | GLU | B | 369 | 43.018 | 74.078 | 72.219 | 1.00 | 42.55 |
| 2965 | C | GLU | B | 369 | 37.668 | 72.248 | 69.966 | 1.00 | 43.17 |
| 2966 | O | GLU | B | 369 | 36.937 | 71.556 | 70.687 | 1.00 | 49.38 |
| 2967 | N | TYR | B | 370 | 37.148 | 73.057 | 69.116 | 1.00 | 45.99 |
| 2968 | CA | TYR | B | 370 | 35.813 | 73.305 | 69.076 | 1.00 | 47.58 |
| 2969 | CB | TYR | B | 370 | 35.422 | 74.286 | 68.072 | 1.00 | 47.27 |
| 2970 | CG | TYR | B | 370 | 35.052 | 75.556 | 68.597 | 1.00 | 52.74 |
| 2971 | CD1 | TYR | B | 370 | 36.064 | 76.635 | 68.641 | 1.00 | 50.10 |
| 2972 | CE1 | TYR | B | 370 | 35.624 | 77.936 | 69.314 | 1.00 | 40.35 |
| 2973 | CZ | TYR | B | 370 | 34.554 | 78.036 | 69.840 | 1.00 | 40.16 |
| 2974 | OH | TYR | B | 370 | 33.991 | 79.170 | 70.276 | 1.00 | 44.26 |
| 2975 | CE2 | TYR | B | 370 | 33.424 | 76.804 | 69.704 | 1.00 | 47.37 |
| 2976 | CD2 | TYR | B | 370 | 33.712 | 75.833 | 69.190 | 1.00 | 42.67 |
| 2977 | C | TYR | B | 370 | 35.030 | 71.874 | 68.684 | 1.00 | 52.99 |
| 2978 | O | TYR | B | 370 | 33.758 | 71.658 | 69.130 | 1.00 | 49.77 |
| 2979 | N | ALA | B | 371 | 35.630 | 71.157 | 67.806 | 1.00 | 47.82 |
| 2980 | CA | ALA | B | 371 | 34.831 | 69.961 | 67.295 | 1.00 | 51.98 |
| 2981 | CB | ALA | B | 371 | 35.207 | 69.678 | 65.859 | 1.00 | 45.87 |
| 2982 | C | ALA | B | 371 | 35.094 | 68.670 | 68.370 | 1.00 | 46.13 |
| 2983 | O | ALA | B | 371 | 34.180 | 68.036 | 68.544 | 1.00 | 48.06 |
| 2984 | N | LEU | B | 372 | 36.211 | 68.555 | 68.932 | 1.00 | 37.70 |
| 2985 | CA | LEU | B | 372 | 36.500 | 67.670 | 69.899 | 1.00 | 40.88 |
| 2986 | CB | LEU | B | 372 | 37.925 | 67.769 | 70.424 | 1.00 | 37.06 |
| 2987 | CG | LEU | B | 372 | 38.958 | 66.880 | 69.768 | 1.00 | 44.88 |
| 2988 | CD1 | LEU | B | 372 | 40.510 | 67.353 | 70.167 | 1.00 | 41.45 |
| 2989 | CD2 | LEU | B | 372 | 38.785 | 65.261 | 70.084 | 1.00 | 38.26 |
| 2990 | C | LEU | B | 372 | 35.631 | 67.922 | 71.168 | 1.00 | 43.20 |
| 2991 | O | LEU | B | 372 | 35.194 | 66.999 | 71.837 | 1.00 | 41.82 |
| 2992 | N | LEU | B | 373 | 35.309 | 69.225 | 71.411 | 1.00 | 42.24 |
| 2993 | CA | LEU | B | 373 | 34.547 | 69.636 | 72.392 | 1.00 | 41.28 |
| 2994 | CB | LEU | B | 373 | 34.686 | 71.195 | 72.706 | 1.00 | 39.69 |
| 2995 | CG | LEU | B | 373 | 33.958 | 71.661 | 74.107 | 1.00 | 41.53 |
| 2996 | CD1 | LEU | B | 373 | 34.524 | 71.057 | 75.268 | 1.00 | 32.76 |
| 2997 | CD2 | LEU | B | 373 | 34.319 | 73.192 | 74.271 | 1.00 | 42.23 |
| 2998 | C | LEU | B | 373 | 33.189 | 69.144 | 72.164 | 1.00 | 40.89 |
| 2999 | O | LEU | B | 373 | 32.526 | 68.647 | 73.086 | 1.00 | 36.48 |
| 3000 | N | ILE | B | 374 | 32.726 | 69.362 | 70.974 | 1.00 | 38.82 |
| 3001 | CA | ILE | B | 374 | 31.222 | 68.979 | 70.736 | 1.00 | 40.41 |
| 3002 | CB | ILE | B | 374 | 30.792 | 69.588 | 69.360 | 1.00 | 49.48 |
| 3003 | CG1 | ILE | B | 374 | 30.806 | 71.010 | 69.376 | 1.00 | 40.21 |
| 3004 | CD1 | ILE | B | 374 | 30.576 | 71.719 | 67.853 | 1.00 | 39.35 |
| 3005 | CG2 | ILE | B | 374 | 29.433 | 69.176 | 68.783 | 1.00 | 50.16 |
| 3006 | C | ILE | B | 374 | 31.071 | 67.463 | 70.633 | 1.00 | 35.65 |
| 3007 | O | ILE | B | 374 | 30.044 | 67.013 | 70.952 | 1.00 | 37.33 |
| 3008 | N | ALA | B | 375 | 32.139 | 66.780 | 70.275 | 1.00 | 34.81 |
| 3009 | CA | ALA | B | 375 | 32.109 | 65.258 | 70.332 | 1.00 | 38.01 |
| 3010 | CB | ALA | B | 375 | 33.550 | 64.740 | 69.719 | 1.00 | 31.42 |
| 3011 | C | ALA | B | 375 | 31.850 | 64.791 | 71.650 | 1.00 | 38.73 |
| 3012 | O | ALA | B | 375 | 31.171 | 63.865 | 71.926 | 1.00 | 35.75 |
| 3013 | N | ILE | B | 376 | 32.786 | 65.389 | 72.622 | 1.00 | 41.09 |
| 3014 | CA | ILE | B | 376 | 32.744 | 65.055 | 74.022 | 1.00 | 35.50 |
| 3015 | CB | ILE | B | 376 | 33.708 | 65.860 | 74.712 | 1.00 | 33.93 |
| 3016 | CG1 | ILE | B | 376 | 35.058 | 65.278 | 74.378 | 1.00 | 37.82 |
| 3017 | CD1 | ILE | B | 376 | 36.260 | 66.085 | 74.970 | 1.00 | 42.61 |
| 3018 | CG2 | ILE | B | 376 | 33.578 | 65.909 | 76.213 | 1.00 | 35.07 |
| 3019 | C | ILE | B | 376 | 31.215 | 65.433 | 74.518 | 1.00 | 39.39 |
| 3020 | O | ILE | B | 376 | 30.600 | 64.662 | 75.226 | 1.00 | 31.63 |
| 3021 | N | ASN | B | 377 | 30.726 | 66.553 | 73.996 | 1.00 | 33.27 |
| 3022 | CA | ASN | B | 377 | 29.481 | 66.963 | 74.402 | 1.00 | 40.51 |
| 3023 | CB | ASN | B | 377 | 29.092 | 68.399 | 73.899 | 1.00 | 35.15 |
| 3024 | CG | ASN | B | 377 | 27.785 | 68.831 | 74.341 | 1.00 | 45.99 |
| 3025 | OD1 | ASN | B | 377 | 26.756 | 68.380 | 73.788 | 1.00 | 57.62 |
| 3026 | ND2 | ASN | B | 377 | 27.628 | 69.868 | 75.129 | 1.00 | 42.62 |
| 3027 | C | ASN | B | 377 | 28.318 | 65.895 | 73.951 | 1.00 | 39.66 |
| 3028 | O | ASN | B | 377 | 27.426 | 65.579 | 74.639 | 1.00 | 33.22 |
| 3029 | N | ILE | B | 378 | 28.475 | 65.476 | 72.689 | 1.00 | 41.09 |
| 3030 | CA | ILE | B | 378 | 27.466 | 64.500 | 72.126 | 1.00 | 36.18 |
| 3031 | CB | ILE | B | 378 | 27.826 | 64.197 | 70.678 | 1.00 | 38.50 |
| 3032 | CG1 | ILE | B | 378 | 27.152 | 65.256 | 69.760 | 1.00 | 35.97 |
| 3033 | CD1 | ILE | B | 378 | 27.776 | 65.473 | 68.277 | 1.00 | 36.60 |
| 3034 | CG2 | ILE | B | 378 | 27.258 | 62.699 | 70.244 | 1.00 | 33.25 |
| 3035 | C | ILE | B | 378 | 27.497 | 63.126 | 72.910 | 1.00 | 35.29 |
| 3036 | O | ILE | B | 378 | 26.423 | 62.693 | 73.370 | 1.00 | 36.52 |
| 3037 | N | PHE | B | 379 | 28.601 | 62.634 | 73.243 | 1.00 | 32.11 |
| 3038 | CA | PHE | B | 379 | 28.708 | 61.503 | 74.070 | 1.00 | 33.81 |
| 3039 | CB | PHE | B | 379 | 30.021 | 60.741 | 73.831 | 1.00 | 32.68 |
| 3040 | CG | PHE | B | 379 | 30.122 | 60.167 | 72.287 | 1.00 | 41.51 |
| 3041 | CD1 | PHE | B | 379 | 29.187 | 59.435 | 71.829 | 1.00 | 38.71 |
| 3042 | CE1 | PHE | B | 379 | 29.260 | 58.986 | 70.315 | 1.00 | 47.93 |
| 3043 | CZ | PHE | B | 379 | 30.147 | 59.236 | 69.593 | 1.00 | 42.87 |
| 3044 | CE2 | PHE | B | 379 | 31.361 | 60.099 | 70.327 | 1.00 | 52.03 |
| 3045 | CD2 | PHE | B | 379 | 31.139 | 60.524 | 71.507 | 1.00 | 40.23 |
| 3046 | C | PHE | B | 379 | 28.527 | 61.768 | 75.664 | 1.00 | 33.12 |
| 3047 | O | PHE | B | 379 | 29.455 | 61.342 | 76.400 | 1.00 | 32.97 |
| 3048 | N | SER | B | 380 | 27.448 | 62.253 | 76.030 | 1.00 | 31.16 |
| 3049 | CA | SER | B | 380 | 27.217 | 62.413 | 77.482 | 1.00 | 33.87 |
| 3050 | CB | SER | B | 380 | 26.593 | 63.847 | 77.671 | 1.00 | 35.32 |
| 3051 | OG | SER | B | 380 | 27.482 | 64.813 | 77.204 | 1.00 | 34.63 |
| 3052 | C | SER | B | 380 | 26.323 | 61.457 | 77.971 | 1.00 | 32.69 |
| 3053 | O | SER | B | 380 | 25.033 | 61.484 | 77.647 | 1.00 | 34.27 |
| 3054 | N | ALA | B | 381 | 26.835 | 60.585 | 78.833 | 1.00 | 31.35 |
| 3055 | CA | ALA | B | 381 | 26.112 | 59.445 | 79.336 | 1.00 | 36.71 |
| 3056 | CB | ALA | B | 381 | 26.981 | 58.621 | 79.982 | 1.00 | 34.88 |
| 3057 | C | ALA | B | 381 | 24.849 | 59.698 | 80.277 | 1.00 | 43.47 |
| 3058 | O | ALA | B | 381 | 23.935 | 58.858 | 80.375 | 1.00 | 39.75 |
| 3059 | N | ASP | B | 382 | 24.757 | 60.907 | 80.854 | 1.00 | 38.77 |
| 3060 | CA | ASP | B | 382 | 23.684 | 61.255 | 81.725 | 1.00 | 37.98 |
| 3061 | CB | ASP | B | 382 | 24.245 | 62.183 | 82.830 | 1.00 | 39.59 |
| 3062 | CG | ASP | B | 382 | 24.706 | 63.467 | 82.317 | 1.00 | 42.80 |
| 3063 | OD1 | ASP | B | 382 | 24.785 | 64.393 | 83.147 | 1.00 | 47.40 |
| 3064 | OD2 | ASP | B | 382 | 24.987 | 63.626 | 81.114 | 1.00 | 42.49 |
| 3065 | C | ASP | B | 382 | 22.458 | 61.983 | 80.969 | 1.00 | 38.07 |
| 3066 | O | ASP | B | 382 | 21.629 | 62.425 | 81.551 | 1.00 | 36.85 |
| 3067 | N | ARG | B | 383 | 22.506 | 61.946 | 79.627 | 1.00 | 33.91 |
| 3068 | CA | ARG | B | 383 | 21.389 | 62.586 | 79.022 | 1.00 | 43.30 |
| 3069 | CB | ARG | B | 383 | 21.483 | 62.676 | 77.426 | 1.00 | 38.18 |
| 3070 | CG | ARG | B | 383 | 22.666 | 63.403 | 76.929 | 1.00 | 38.53 |
| 3071 | CD | ARG | B | 383 | 22.759 | 64.827 | 77.504 | 1.00 | 35.18 |
| 3072 | NE | ARG | B | 383 | 23.761 | 65.525 | 76.860 | 1.00 | 37.54 |
| 3073 | CZ | ARG | B | 383 | 23.986 | 66.862 | 76.877 | 1.00 | 40.86 |
| 3074 | NH1 | ARG | B | 383 | 23.246 | 67.593 | 77.627 | 1.00 | 31.00 |
| 3075 | NH2 | ARG | B | 383 | 25.014 | 67.372 | 76.123 | 1.00 | 33.65 |
| 3076 | C | ARG | B | 383 | 20.092 | 61.718 | 79.407 | 1.00 | 41.21 |
| 3077 | O | ARG | B | 383 | 20.239 | 60.515 | 79.738 | 1.00 | 42.43 |
| 3078 | N | PRO | B | 384 | 18.965 | 62.322 | 79.384 | 1.00 | 40.98 |
| 3079 | CA | PRO | B | 384 | 17.715 | 61.469 | 79.656 | 1.00 | 42.66 |
| 3080 | CB | PRO | B | 384 | 16.564 | 62.496 | 79.569 | 1.00 | 44.34 |
| 3081 | CG | PRO | B | 384 | 17.305 | 63.907 | 79.871 | 1.00 | 38.75 |
| 3082 | CD | PRO | B | 384 | 18.605 | 63.775 | 79.084 | 1.00 | 37.14 |
| 3083 | C | PRO | B | 384 | 17.496 | 60.418 | 78.613 | 1.00 | 40.57 |
| 3084 | O | PRO | B | 384 | 17.827 | 60.633 | 77.482 | 1.00 | 40.45 |
| 3085 | N | ASN | B | 385 | 16.974 | 59.352 | 79.082 | 1.00 | 43.30 |
| 3086 | CA | ASN | B | 385 | 16.593 | 58.197 | 78.182 | 1.00 | 48.11 |
| 3087 | CB | ASN | B | 385 | 15.585 | 58.541 | 77.140 | 1.00 | 45.42 |
| 3088 | CG | ASN | B | 385 | 14.505 | 59.439 | 77.724 | 1.00 | 52.65 |
| 3089 | OD1 | ASN | B | 385 | 13.873 | 59.126 | 78.665 | 1.00 | 55.72 |
| 3090 | ND2 | ASN | B | 385 | 14.327 | 60.658 | 77.178 | 1.00 | 54.76 |
| 3091 | C | ASN | B | 385 | 17.816 | 57.479 | 77.451 | 1.00 | 48.64 |
| 3092 | O | ASN | B | 385 | 17.523 | 56.696 | 76.493 | 1.00 | 43.31 |
| 3093 | N | VAL | B | 386 | 19.021 | 57.602 | 77.984 | 1.00 | 41.33 |
| 3094 | CA | VAL | B | 386 | 20.060 | 56.863 | 77.388 | 1.00 | 42.45 |
| 3095 | CB | VAL | B | 386 | 21.433 | 57.459 | 77.739 | 1.00 | 41.84 |
| 3096 | CG1 | VAL | B | 386 | 22.550 | 56.396 | 77.457 | 1.00 | 36.52 |
| 3097 | CG2 | VAL | B | 386 | 21.585 | 58.739 | 76.787 | 1.00 | 48.51 |
| 3098 | C | VAL | B | 386 | 19.869 | 55.483 | 77.822 | 1.00 | 47.92 |
| 3099 | O | VAL | B | 386 | 19.865 | 55.185 | 78.942 | 1.00 | 52.17 |
| 3100 | N | GLN | B | 387 | 19.968 | 54.486 | 76.939 | 1.00 | 51.58 |
| 3101 | CA | GLN | B | 387 | 19.793 | 53.147 | 77.402 | 1.00 | 48.85 |
| 3102 | CB | GLN | B | 387 | 18.917 | 52.374 | 76.350 | 1.00 | 53.71 |
| 3103 | CG | GLN | B | 387 | 17.442 | 52.809 | 76.192 | 1.00 | 53.80 |
| 3104 | CD | GLN | B | 387 | 16.614 | 51.865 | 75.515 | 1.00 | 63.34 |
| 3105 | OE1 | GLN | B | 387 | 16.321 | 50.688 | 76.001 | 1.00 | 66.39 |
| 3106 | NE2 | GLN | B | 387 | 16.200 | 52.255 | 74.301 | 1.00 | 72.65 |
| 3107 | C | GLN | B | 387 | 20.918 | 52.350 | 77.686 | 1.00 | 51.24 |
| 3108 | O | GLN | B | 387 | 20.837 | 51.407 | 78.567 | 1.00 | 51.47 |
| 3109 | N | GLU | B | 388 | 22.116 | 52.752 | 77.087 | 1.00 | 49.23 |
| 3110 | CA | GLU | B | 388 | 23.346 | 52.227 | 77.309 | 1.00 | 44.52 |
| 3111 | CB | GLU | B | 388 | 23.934 | 51.490 | 76.035 | 1.00 | 48.85 |
| 3112 | CG | GLU | B | 388 | 22.981 | 50.173 | 75.625 | 1.00 | 58.45 |
| 3113 | CD | GLU | B | 388 | 23.286 | 49.659 | 74.132 | 1.00 | 60.60 |
| 3114 | OE1 | GLU | B | 388 | 22.905 | 48.548 | 73.897 | 1.00 | 77.84 |
| 3115 | OE2 | GLU | B | 388 | 23.680 | 50.333 | 73.228 | 1.00 | 61.44 |
| 3116 | C | GLU | B | 388 | 24.358 | 53.159 | 77.798 | 1.00 | 44.83 |
| 3117 | O | GLU | B | 388 | 25.470 | 53.409 | 77.170 | 1.00 | 48.61 |
| 3118 | N | PRO | B | 389 | 24.190 | 53.716 | 78.934 | 1.00 | 47.42 |
| 3119 | CA | PRO | B | 389 | 25.178 | 54.732 | 79.457 | 1.00 | 39.92 |
| 3120 | CB | PRO | B | 389 | 24.641 | 55.108 | 80.732 | 1.00 | 40.08 |
| 3121 | CG | PRO | B | 389 | 23.777 | 53.909 | 81.212 | 1.00 | 47.99 |
| 3122 | CD | PRO | B | 389 | 23.093 | 53.436 | 79.867 | 1.00 | 41.25 |
| 3123 | C | PRO | B | 389 | 26.597 | 54.326 | 79.438 | 1.00 | 42.93 |
| 3124 | O | PRO | B | 389 | 27.529 | 55.133 | 79.280 | 1.00 | 41.55 |
| 3125 | N | GLY | B | 390 | 26.883 | 53.146 | 79.869 | 1.00 | 49.43 |
| 3126 | CA | GLY | B | 390 | 28.215 | 52.662 | 80.145 | 1.00 | 43.80 |
| 3127 | C | GLY | B | 390 | 28.907 | 52.801 | 78.721 | 1.00 | 51.10 |
| 3128 | O | GLY | B | 390 | 30.087 | 53.361 | 78.713 | 1.00 | 50.45 |
| 3129 | N | ARG | B | 391 | 28.315 | 52.449 | 77.669 | 1.00 | 46.22 |
| 3130 | CA | ARG | B | 391 | 28.835 | 52.490 | 76.388 | 1.00 | 49.67 |
| 3131 | CB | ARG | B | 391 | 27.789 | 51.840 | 75.294 | 1.00 | 50.81 |
| 3132 | CG | ARG | B | 391 | 28.265 | 50.637 | 74.672 | 1.00 | 62.59 |
| 3133 | CD | ARG | B | 391 | 27.148 | 50.063 | 73.688 | 1.00 | 57.61 |
| 3134 | NE | ARG | B | 391 | 27.542 | 50.198 | 72.346 | 1.00 | 60.34 |
| 3135 | CZ | ARG | B | 391 | 26.712 | 49.956 | 71.336 | 1.00 | 65.60 |
| 3136 | NH1 | ARG | B | 391 | 25.489 | 49.448 | 71.571 | 1.00 | 63.01 |
| 3137 | NH2 | ARG | B | 391 | 27.137 | 50.044 | 70.028 | 1.00 | 59.69 |
| 3138 | C | ARG | B | 391 | 29.054 | 53.880 | 75.931 | 1.00 | 48.48 |
| 3139 | O | ARG | B | 391 | 30.203 | 54.230 | 75.317 | 1.00 | 49.56 |
| 3140 | N | VAL | B | 392 | 28.120 | 54.741 | 76.158 | 1.00 | 48.12 |
| 3141 | CA | VAL | B | 392 | 28.343 | 56.259 | 75.744 | 1.00 | 47.83 |
| 3142 | CB | VAL | B | 392 | 27.182 | 56.986 | 76.124 | 1.00 | 48.35 |
| 3143 | CG1 | VAL | B | 392 | 27.339 | 58.600 | 75.950 | 1.00 | 45.88 |
| 3144 | CG2 | VAL | B | 392 | 25.878 | 56.348 | 75.321 | 1.00 | 42.60 |
| 3145 | C | VAL | B | 392 | 29.594 | 56.730 | 76.511 | 1.00 | 43.63 |
| 3146 | O | VAL | B | 392 | 30.478 | 57.348 | 75.804 | 1.00 | 45.69 |
| 3147 | N | GLU | B | 393 | 29.745 | 56.411 | 77.768 | 1.00 | 37.90 |
| 3148 | CA | GLU | B | 393 | 30.705 | 56.936 | 78.557 | 1.00 | 42.47 |
| 3149 | CB | GLU | B | 393 | 30.552 | 56.493 | 80.014 | 1.00 | 46.80 |
| 3150 | CG | GLU | B | 393 | 31.086 | 57.536 | 81.110 | 1.00 | 52.70 |
| 3151 | CD | GLU | B | 393 | 30.912 | 56.905 | 82.636 | 1.00 | 62.94 |
| 3152 | OE1 | GLU | B | 393 | 29.985 | 55.972 | 82.850 | 1.00 | 58.05 |
| 3153 | OE2 | GLU | B | 393 | 31.558 | 57.350 | 83.570 | 1.00 | 57.70 |
| 3154 | C | GLU | B | 393 | 32.070 | 56.368 | 78.029 | 1.00 | 49.81 |
| 3155 | O | GLU | B | 393 | 33.100 | 56.998 | 78.094 | 1.00 | 46.21 |
| 3156 | N | ALA | B | 394 | 32.036 | 55.065 | 77.599 | 1.00 | 46.59 |
| 3157 | CA | ALA | B | 394 | 33.089 | 54.500 | 77.027 | 1.00 | 51.72 |
| 3158 | CB | ALA | B | 394 | 32.849 | 52.975 | 76.829 | 1.00 | 47.93 |
| 3159 | C | ALA | B | 394 | 33.426 | 55.225 | 75.548 | 1.00 | 49.33 |
| 3160 | O | ALA | B | 394 | 34.571 | 55.308 | 75.172 | 1.00 | 53.93 |
| 3161 | N | LEU | B | 395 | 32.512 | 55.542 | 74.841 | 1.00 | 47.24 |
| 3162 | CA | LEU | B | 395 | 32.713 | 56.174 | 73.588 | 1.00 | 47.77 |
| 3163 | CB | LEU | B | 395 | 31.375 | 56.420 | 73.002 | 1.00 | 47.27 |
| 3164 | CG | LEU | B | 395 | 30.650 | 55.089 | 72.518 | 1.00 | 52.97 |
| 3165 | CD1 | LEU | B | 395 | 29.486 | 55.532 | 71.568 | 1.00 | 56.50 |
| 3166 | CD2 | LEU | B | 395 | 31.633 | 54.369 | 71.629 | 1.00 | 65.08 |
| 3167 | C | LEU | B | 395 | 33.351 | 57.635 | 73.853 | 1.00 | 50.33 |
| 3168 | O | LEU | B | 395 | 34.224 | 57.986 | 73.190 | 1.00 | 43.23 |
| 3169 | N | GLN | B | 396 | 32.857 | 58.316 | 74.935 | 1.00 | 45.97 |
| 3170 | CA | GLN | B | 396 | 33.453 | 59.568 | 75.249 | 1.00 | 45.92 |
| 3171 | CB | GLN | B | 396 | 32.549 | 60.360 | 76.332 | 1.00 | 48.46 |
| 3172 | CG | GLN | B | 396 | 33.013 | 61.694 | 76.726 | 1.00 | 38.06 |
| 3173 | CD | GLN | B | 396 | 32.178 | 62.350 | 77.874 | 1.00 | 43.58 |
| 3174 | OE1 | GLN | B | 396 | 31.218 | 63.093 | 77.571 | 1.00 | 37.96 |
| 3175 | NE2 | GLN | B | 396 | 32.370 | 61.932 | 79.033 | 1.00 | 33.42 |
| 3176 | C | GLN | B | 396 | 34.794 | 59.728 | 75.616 | 1.00 | 39.07 |
| 3177 | O | GLN | B | 396 | 35.399 | 60.637 | 75.344 | 1.00 | 46.93 |
| 3178 | N | GLN | B | 397 | 35.306 | 58.840 | 76.278 | 1.00 | 43.51 |
| 3179 | CA | GLN | B | 397 | 36.502 | 58.774 | 76.766 | 1.00 | 45.19 |
| 3180 | CB | GLN | B | 397 | 36.753 | 57.518 | 77.438 | 1.00 | 50.22 |
| 3181 | CG | GLN | B | 397 | 38.002 | 57.491 | 78.398 | 1.00 | 53.48 |
| 3182 | CD | GLN | B | 397 | 38.113 | 56.049 | 79.202 | 1.00 | 74.85 |
| 3183 | OE1 | GLN | B | 397 | 37.123 | 55.689 | 79.894 | 1.00 | 73.18 |
| 3184 | NE2 | GLN | B | 397 | 39.234 | 55.224 | 78.998 | 1.00 | 69.16 |
| 3185 | C | GLN | B | 397 | 37.687 | 59.178 | 75.868 | 1.00 | 52.84 |
| 3186 | O | GLN | B | 397 | 38.635 | 60.070 | 76.246 | 1.00 | 49.93 |
| 3187 | N | PRO | B | 398 | 37.842 | 58.514 | 74.840 | 1.00 | 53.73 |
| 3188 | CA | PRO | B | 398 | 38.993 | 58.790 | 74.001 | 1.00 | 55.88 |
| 3189 | CB | PRO | B | 398 | 38.763 | 57.666 | 72.715 | 1.00 | 62.63 |
| 3190 | CG | PRO | B | 398 | 37.129 | 57.322 | 72.859 | 1.00 | 53.57 |
| 3191 | CD | PRO | B | 398 | 37.052 | 57.343 | 74.422 | 1.00 | 58.05 |
| 3192 | C | PRO | B | 398 | 38.851 | 60.214 | 73.388 | 1.00 | 52.09 |
| 3193 | O | PRO | B | 398 | 39.793 | 60.773 | 72.860 | 1.00 | 52.37 |
| 3194 | N | TYR | B | 399 | 37.751 | 60.840 | 73.430 | 1.00 | 52.85 |
| 3195 | CA | TYR | B | 399 | 37.678 | 62.200 | 72.875 | 1.00 | 50.56 |
| 3196 | CB | TYR | B | 399 | 36.329 | 62.567 | 72.524 | 1.00 | 47.48 |
| 3197 | CG | TYR | B | 399 | 35.877 | 61.893 | 71.280 | 1.00 | 58.67 |
| 3198 | CD1 | TYR | B | 399 | 36.390 | 62.258 | 69.997 | 1.00 | 58.36 |
| 3199 | CE1 | TYR | B | 399 | 36.185 | 61.595 | 68.906 | 1.00 | 57.41 |
| 3200 | CZ | TYR | B | 399 | 35.378 | 60.455 | 68.910 | 1.00 | 65.70 |
| 3201 | OH | TYR | B | 399 | 35.023 | 59.768 | 67.846 | 1.00 | 56.50 |
| 3202 | CE2 | TYR | B | 399 | 34.729 | 60.113 | 70.174 | 1.00 | 69.32 |
| 3203 | CD2 | TYR | B | 399 | 35.052 | 60.821 | 71.309 | 1.00 | 64.30 |
| 3204 | C | TYR | B | 399 | 38.168 | 63.070 | 74.069 | 1.00 | 54.09 |
| 3205 | O | TYR | B | 399 | 38.825 | 64.123 | 73.739 | 1.00 | 57.36 |
| 3206 | N | VAL | B | 400 | 37.965 | 62.696 | 75.271 | 1.00 | 45.95 |
| 3207 | CA | VAL | B | 400 | 38.468 | 63.498 | 76.324 | 1.00 | 45.82 |
| 3208 | CB | VAL | B | 400 | 37.815 | 63.108 | 77.785 | 1.00 | 38.52 |
| 3209 | CG1 | VAL | B | 400 | 38.430 | 63.876 | 78.843 | 1.00 | 38.05 |
| 3210 | CG2 | VAL | B | 400 | 36.331 | 63.403 | 77.774 | 1.00 | 42.03 |
| 3211 | C | VAL | B | 400 | 39.920 | 63.465 | 76.392 | 1.00 | 43.40 |
| 3212 | O | VAL | B | 400 | 40.539 | 64.426 | 76.892 | 1.00 | 41.68 |
| 3213 | N | GLU | B | 401 | 40.454 | 62.287 | 76.118 | 1.00 | 42.91 |
| 3214 | CA | GLU | B | 401 | 41.801 | 62.093 | 76.259 | 1.00 | 46.81 |
| 3215 | CB | GLU | B | 401 | 42.164 | 60.569 | 76.197 | 1.00 | 50.70 |
| 3216 | CG | GLU | B | 401 | 43.592 | 60.205 | 76.576 | 1.00 | 57.12 |
| 3217 | CD | GLU | B | 401 | 43.723 | 58.785 | 77.282 | 1.00 | 69.56 |
| 3218 | OE1 | GLU | B | 401 | 43.083 | 57.753 | 76.734 | 1.00 | 72.77 |
| 3219 | OE2 | GLU | B | 401 | 44.364 | 58.632 | 78.277 | 1.00 | 71.00 |
| 3220 | C | GLU | B | 401 | 42.614 | 62.830 | 75.112 | 1.00 | 43.66 |
| 3221 | O | GLU | B | 401 | 43.608 | 63.397 | 75.287 | 1.00 | 41.67 |
| 3222 | N | ALA | B | 402 | 41.947 | 62.883 | 74.025 | 1.00 | 48.95 |
| 3223 | CA | ALA | B | 402 | 42.520 | 63.600 | 72.910 | 1.00 | 51.49 |
| 3224 | CB | ALA | B | 402 | 41.662 | 63.395 | 71.667 | 1.00 | 45.05 |
| 3225 | C | ALA | B | 402 | 42.622 | 64.979 | 73.224 | 1.00 | 46.67 |
| 3226 | O | ALA | B | 402 | 43.676 | 65.642 | 72.938 | 1.00 | 47.78 |
| 3227 | N | LEU | B | 403 | 41.518 | 65.517 | 73.675 | 1.00 | 44.41 |
| 3228 | CA | LEU | B | 403 | 41.438 | 66.964 | 74.065 | 1.00 | 42.46 |
| 3229 | CB | LEU | B | 403 | 40.015 | 67.443 | 74.295 | 1.00 | 33.75 |
| 3230 | CG | LEU | B | 403 | 39.846 | 68.871 | 74.540 | 1.00 | 42.51 |
| 3231 | CD1 | LEU | B | 403 | 40.537 | 69.597 | 73.404 | 1.00 | 37.35 |
| 3232 | CD2 | LEU | B | 403 | 38.403 | 69.363 | 74.740 | 1.00 | 28.87 |
| 3233 | C | LEU | B | 403 | 42.509 | 67.341 | 75.098 | 1.00 | 43.11 |
| 3234 | O | LEU | B | 403 | 43.206 | 68.350 | 74.938 | 1.00 | 46.62 |
| 3235 | N | LEU | B | 404 | 42.741 | 66.497 | 76.077 | 1.00 | 43.05 |
| 3236 | CA | LEU | B | 404 | 43.682 | 66.687 | 77.057 | 1.00 | 43.92 |
| 3237 | CB | LEU | B | 404 | 43.621 | 65.709 | 78.122 | 1.00 | 42.57 |
| 3238 | CG | LEU | B | 404 | 44.754 | 65.651 | 79.179 | 1.00 | 49.81 |
| 3239 | CD1 | LEU | B | 404 | 44.964 | 67.122 | 79.790 | 1.00 | 49.83 |
| 3240 | CD2 | LEU | B | 404 | 44.445 | 64.698 | 80.172 | 1.00 | 53.06 |
| 3241 | C | LEU | B | 404 | 45.172 | 66.804 | 76.401 | 1.00 | 51.79 |
| 3242 | O | LEU | B | 404 | 46.052 | 67.766 | 76.669 | 1.00 | 48.42 |
| 3243 | N | SER | B | 405 | 45.506 | 65.747 | 75.777 | 1.00 | 50.35 |
| 3244 | CA | SER | B | 405 | 46.902 | 65.745 | 75.119 | 1.00 | 56.85 |
| 3245 | CB | SER | B | 405 | 47.000 | 64.285 | 74.387 | 1.00 | 48.60 |
| 3246 | OG | SER | B | 405 | 46.226 | 64.556 | 73.285 | 1.00 | 58.57 |
| 3247 | C | SER | B | 405 | 47.022 | 66.867 | 74.169 | 1.00 | 50.98 |
| 3248 | O | SER | B | 405 | 47.920 | 67.578 | 74.326 | 1.00 | 55.26 |
| 3249 | N | TYR | B | 406 | 46.086 | 67.082 | 73.349 | 1.00 | 48.23 |
| 3250 | CA | TYR | B | 406 | 46.173 | 68.198 | 72.461 | 1.00 | 51.31 |
| 3251 | CB | TYR | B | 406 | 44.919 | 68.339 | 71.790 | 1.00 | 47.60 |
| 3252 | CG | TYR | B | 406 | 44.890 | 69.267 | 70.640 | 1.00 | 59.55 |
| 3253 | CD1 | TYR | B | 406 | 45.552 | 68.953 | 69.407 | 1.00 | 55.72 |
| 3254 | CE1 | TYR | B | 406 | 45.630 | 69.909 | 68.299 | 1.00 | 55.02 |
| 3255 | CZ | TYR | B | 406 | 44.898 | 71.017 | 68.355 | 1.00 | 63.04 |
| 3256 | OH | TYR | B | 406 | 44.731 | 71.941 | 67.433 | 1.00 | 63.15 |
| 3257 | CE2 | TYR | B | 406 | 44.115 | 71.303 | 69.606 | 1.00 | 62.97 |
| 3258 | CD2 | TYR | B | 406 | 44.098 | 70.473 | 70.601 | 1.00 | 58.74 |
| 3259 | C | TYR | B | 406 | 46.444 | 69.486 | 73.174 | 1.00 | 58.44 |
| 3260 | O | TYR | B | 406 | 47.480 | 70.166 | 72.935 | 1.00 | 62.89 |
| 3261 | N | THR | B | 407 | 45.620 | 69.978 | 74.099 | 1.00 | 58.58 |
| 3262 | CA | THR | B | 407 | 45.817 | 71.225 | 74.797 | 1.00 | 48.27 |
| 3263 | CB | THR | B | 407 | 44.573 | 71.502 | 75.818 | 1.00 | 43.99 |
| 3264 | OG1 | THR | B | 407 | 44.442 | 70.504 | 76.852 | 1.00 | 44.98 |
| 3265 | CG2 | THR | B | 407 | 43.383 | 71.691 | 75.113 | 1.00 | 42.33 |
| 3266 | C | THR | B | 407 | 47.056 | 71.188 | 75.527 | 1.00 | 46.17 |
| 3267 | O | THR | B | 407 | 47.666 | 72.267 | 75.718 | 1.00 | 58.76 |
| 3268 | N | ARG | B | 408 | 47.555 | 70.144 | 75.977 | 1.00 | 50.52 |
| 3269 | CA | ARG | B | 408 | 48.814 | 70.143 | 76.616 | 1.00 | 57.73 |
| 3270 | CB | ARG | B | 408 | 49.161 | 68.795 | 77.190 | 1.00 | 60.99 |
| 3271 | CG | ARG | B | 408 | 49.267 | 68.643 | 78.710 | 1.00 | 65.94 |
| 3272 | CD | ARG | B | 408 | 50.252 | 69.792 | 79.272 | 1.00 | 91.42 |
| 3273 | NE | ARG | B | 408 | 51.660 | 69.419 | 79.440 | 1.00 | 88.13 |
| 3274 | CZ | ARG | B | 408 | 52.676 | 70.329 | 79.553 | 1.00 | 97.15 |
| 3275 | NH1 | ARG | B | 408 | 52.483 | 71.701 | 79.541 | 1.00 | 91.28 |
| 3276 | NH2 | ARG | B | 408 | 53.930 | 69.930 | 79.739 | 1.00 | 91.45 |
| 3277 | C | ARG | B | 408 | 49.940 | 70.584 | 75.468 | 1.00 | 63.60 |
| 3278 | O | ARG | B | 408 | 50.910 | 71.225 | 75.761 | 1.00 | 66.13 |
| 3279 | N | ILE | B | 409 | 49.651 | 70.297 | 74.231 | 1.00 | 59.66 |
| 3280 | CA | ILE | B | 409 | 50.375 | 70.830 | 73.100 | 1.00 | 62.29 |
| 3281 | CB | ILE | B | 409 | 50.109 | 69.867 | 71.770 | 1.00 | 57.98 |
| 3282 | CG1 | ILE | B | 409 | 50.780 | 68.550 | 72.071 | 1.00 | 52.77 |
| 3283 | CD1 | ILE | B | 409 | 50.198 | 67.240 | 70.873 | 1.00 | 57.84 |
| 3284 | CG2 | ILE | B | 409 | 50.806 | 70.472 | 70.532 | 1.00 | 59.90 |
| 3285 | C | ILE | B | 409 | 50.166 | 72.217 | 72.767 | 1.00 | 58.83 |
| 3286 | O | ILE | B | 409 | 51.090 | 72.968 | 72.861 | 1.00 | 63.13 |
| 3287 | N | LYS | B | 410 | 48.995 | 72.605 | 72.380 | 1.00 | 56.21 |
| 3288 | CA | LYS | B | 410 | 48.801 | 74.031 | 72.085 | 1.00 | 57.56 |
| 3289 | CB | LYS | B | 410 | 47.315 | 74.340 | 71.778 | 1.00 | 61.31 |
| 3290 | CG | LYS | B | 410 | 46.801 | 75.771 | 71.712 | 1.00 | 57.24 |
| 3291 | CD | LYS | B | 410 | 45.494 | 75.911 | 71.004 | 1.00 | 54.26 |
| 3292 | CE | LYS | B | 410 | 45.245 | 77.070 | 70.613 | 1.00 | 49.91 |
| 3293 | NZ | LYS | B | 410 | 44.007 | 77.304 | 69.876 | 1.00 | 63.13 |
| 3294 | C | LYS | B | 410 | 49.321 | 75.022 | 73.028 | 1.00 | 62.14 |
| 3295 | O | LYS | B | 410 | 50.108 | 75.881 | 72.807 | 1.00 | 65.63 |
| 3296 | N | ARG | B | 411 | 48.891 | 74.979 | 74.236 | 1.00 | 68.06 |
| 3297 | CA | ARG | B | 411 | 49.301 | 75.944 | 75.310 | 1.00 | 59.35 |
| 3298 | CB | ARG | B | 411 | 48.220 | 76.860 | 75.573 | 1.00 | 55.30 |
| 3299 | CG | ARG | B | 411 | 48.481 | 78.414 | 74.976 | 1.00 | 64.39 |
| 3300 | CD | ARG | B | 411 | 47.344 | 78.915 | 74.172 | 1.00 | 56.25 |
| 3301 | NE | ARG | B | 411 | 47.810 | 79.128 | 72.848 | 1.00 | 72.33 |
| 3302 | CZ | ARG | B | 411 | 46.991 | 79.535 | 71.859 | 1.00 | 80.77 |
| 3303 | NH1 | ARG | B | 411 | 45.616 | 79.983 | 72.098 | 1.00 | 76.20 |
| 3304 | NH2 | ARG | B | 411 | 47.548 | 79.604 | 70.599 | 1.00 | 82.01 |
| 3305 | C | ARG | B | 411 | 49.766 | 75.174 | 76.459 | 1.00 | 61.22 |
| 3306 | O | ARG | B | 411 | 49.183 | 74.996 | 77.456 | 1.00 | 65.25 |
| 3307 | N | PRO | B | 412 | 50.972 | 74.672 | 76.397 | 1.00 | 65.47 |
| 3308 | CA | PRO | B | 412 | 51.624 | 73.929 | 77.427 | 1.00 | 56.72 |
| 3309 | CB | PRO | B | 412 | 53.026 | 73.570 | 76.677 | 1.00 | 59.83 |
| 3310 | CG | PRO | B | 412 | 53.182 | 74.705 | 75.669 | 1.00 | 49.87 |
| 3311 | CD | PRO | B | 412 | 51.812 | 74.717 | 75.070 | 1.00 | 62.38 |
| 3312 | C | PRO | B | 412 | 51.804 | 74.467 | 78.748 | 1.00 | 62.03 |
| 3313 | O | PRO | B | 412 | 52.101 | 73.670 | 79.764 | 1.00 | 62.16 |
| 3314 | N | GLN | B | 413 | 52.033 | 75.746 | 78.871 | 1.00 | 62.83 |
| 3315 | CA | GLN | B | 413 | 52.334 | 76.137 | 80.246 | 1.00 | 63.94 |
| 3316 | CB | GLN | B | 413 | 53.399 | 77.310 | 80.331 | 1.00 | 64.15 |
| 3317 | CG | GLN | B | 413 | 54.633 | 76.893 | 81.238 | 1.00 | 63.58 |
| 3318 | CD | GLN | B | 413 | 55.637 | 75.992 | 80.326 | 1.00 | 76.45 |
| 3319 | OE1 | GLN | B | 413 | 56.098 | 74.947 | 80.815 | 1.00 | 71.82 |
| 3320 | NE2 | GLN | B | 413 | 55.851 | 76.315 | 79.015 | 1.00 | 75.29 |
| 3321 | C | GLN | B | 413 | 50.988 | 76.542 | 80.967 | 1.00 | 65.02 |
| 3322 | O | GLN | B | 413 | 50.959 | 77.209 | 82.072 | 1.00 | 67.96 |
| 3323 | N | ASP | B | 414 | 49.921 | 76.361 | 80.290 | 1.00 | 61.61 |
| 3324 | CA | ASP | B | 414 | 48.591 | 76.678 | 80.888 | 1.00 | 55.66 |
| 3325 | CB | ASP | B | 414 | 47.875 | 77.673 | 80.044 | 1.00 | 52.94 |
| 3326 | CG | ASP | B | 414 | 46.618 | 78.283 | 80.746 | 1.00 | 58.90 |
| 3327 | OD1 | ASP | B | 414 | 45.845 | 79.161 | 80.143 | 1.00 | 56.74 |
| 3328 | OD2 | ASP | B | 414 | 46.388 | 77.828 | 81.913 | 1.00 | 63.67 |
| 3329 | C | ASP | B | 414 | 47.819 | 75.431 | 81.040 | 1.00 | 47.48 |
| 3330 | O | ASP | B | 414 | 47.038 | 75.146 | 80.163 | 1.00 | 48.35 |
| 3331 | N | GLN | B | 415 | 48.057 | 74.745 | 82.083 | 1.00 | 47.21 |
| 3332 | CA | GLN | B | 415 | 47.333 | 73.518 | 82.410 | 1.00 | 48.12 |
| 3333 | CB | GLN | B | 415 | 47.994 | 72.750 | 83.478 | 1.00 | 45.31 |
| 3334 | CG | GLN | B | 415 | 49.319 | 72.153 | 82.945 | 1.00 | 67.82 |
| 3335 | CD | GLN | B | 415 | 50.253 | 71.461 | 84.067 | 1.00 | 89.11 |
| 3336 | OE1 | GLN | B | 415 | 51.322 | 70.767 | 83.723 | 1.00 | 85.93 |
| 3337 | NE2 | GLN | B | 415 | 49.893 | 71.715 | 85.443 | 1.00 | 83.43 |
| 3338 | C | GLN | B | 415 | 45.892 | 73.670 | 82.784 | 1.00 | 49.96 |
| 3339 | O | GLN | B | 415 | 45.079 | 72.671 | 82.694 | 1.00 | 43.98 |
| 3340 | N | LEU | B | 416 | 45.501 | 74.945 | 83.123 | 1.00 | 47.10 |
| 3341 | CA | LEU | B | 416 | 43.978 | 75.120 | 83.503 | 1.00 | 46.67 |
| 3342 | CB | LEU | B | 416 | 43.862 | 76.220 | 84.464 | 1.00 | 42.24 |
| 3343 | CG | LEU | B | 416 | 44.829 | 76.024 | 85.695 | 1.00 | 46.70 |
| 3344 | CD1 | LEU | B | 416 | 44.294 | 77.038 | 86.573 | 1.00 | 50.30 |
| 3345 | CD2 | LEU | B | 416 | 44.747 | 74.612 | 86.360 | 1.00 | 47.05 |
| 3346 | C | LEU | B | 416 | 43.141 | 75.423 | 82.359 | 1.00 | 47.55 |
| 3347 | O | LEU | B | 416 | 41.901 | 75.394 | 82.489 | 1.00 | 46.19 |
| 3348 | N | ARG | B | 417 | 43.750 | 75.643 | 81.152 | 1.00 | 43.61 |
| 3349 | CA | ARG | B | 417 | 42.849 | 75.985 | 79.953 | 1.00 | 45.54 |
| 3350 | CB | ARG | B | 417 | 43.772 | 76.394 | 78.800 | 1.00 | 47.18 |
| 3351 | CG | ARG | B | 417 | 43.003 | 76.958 | 77.607 | 1.00 | 52.36 |
| 3352 | CD | ARG | B | 417 | 44.016 | 77.709 | 76.629 | 1.00 | 43.04 |
| 3353 | NE | ARG | B | 417 | 43.332 | 77.806 | 75.322 | 1.00 | 48.08 |
| 3354 | CZ | ARG | B | 417 | 43.399 | 76.764 | 74.337 | 1.00 | 41.87 |
| 3355 | NH1 | ARG | B | 417 | 43.918 | 75.624 | 74.633 | 1.00 | 27.87 |
| 3356 | NH2 | ARG | B | 417 | 42.884 | 77.023 | 73.189 | 1.00 | 42.80 |
| 3357 | C | ARG | B | 417 | 41.925 | 74.750 | 79.581 | 1.00 | 44.39 |
| 3358 | O | ARG | B | 417 | 40.805 | 74.921 | 79.177 | 1.00 | 41.74 |
| 3359 | N | PHE | B | 418 | 42.410 | 73.546 | 79.764 | 1.00 | 41.38 |
| 3360 | CA | PHE | B | 418 | 41.673 | 72.442 | 79.433 | 1.00 | 44.48 |
| 3361 | CB | PHE | B | 418 | 42.645 | 71.168 | 79.588 | 1.00 | 38.64 |
| 3362 | CG | PHE | B | 418 | 41.886 | 69.814 | 79.523 | 1.00 | 40.60 |
| 3363 | CD1 | PHE | B | 418 | 41.185 | 69.445 | 78.460 | 1.00 | 46.74 |
| 3364 | CE1 | PHE | B | 418 | 40.649 | 68.249 | 78.287 | 1.00 | 37.85 |
| 3365 | CZ | PHE | B | 418 | 40.741 | 67.416 | 79.212 | 1.00 | 42.90 |
| 3366 | CE2 | PHE | B | 418 | 41.415 | 67.737 | 80.358 | 1.00 | 50.83 |
| 3367 | CD2 | PHE | B | 418 | 41.986 | 68.922 | 80.503 | 1.00 | 45.06 |
| 3368 | C | PHE | B | 418 | 40.423 | 72.254 | 80.431 | 1.00 | 38.51 |
| 3369 | O | PHE | B | 418 | 39.257 | 72.212 | 80.003 | 1.00 | 38.19 |
| 3370 | N | PRO | B | 419 | 40.670 | 72.255 | 81.676 | 1.00 | 39.54 |
| 3371 | CA | PRO | B | 419 | 39.392 | 72.192 | 82.585 | 1.00 | 34.08 |
| 3372 | CB | PRO | B | 419 | 40.078 | 72.081 | 84.039 | 1.00 | 32.89 |
| 3373 | CG | PRO | B | 419 | 41.394 | 72.736 | 83.884 | 1.00 | 33.97 |
| 3374 | CD | PRO | B | 419 | 41.834 | 72.229 | 82.436 | 1.00 | 36.76 |
| 3375 | C | PRO | B | 419 | 38.514 | 73.382 | 82.451 | 1.00 | 33.49 |
| 3376 | O | PRO | B | 419 | 37.294 | 73.252 | 82.531 | 1.00 | 30.11 |
| 3377 | N | ARG | B | 420 | 39.024 | 74.565 | 82.149 | 1.00 | 34.06 |
| 3378 | CA | ARG | B | 420 | 38.014 | 75.588 | 81.942 | 1.00 | 40.49 |
| 3379 | CB | ARG | B | 420 | 38.491 | 77.117 | 81.933 | 1.00 | 32.76 |
| 3380 | CG | ARG | B | 420 | 39.683 | 77.385 | 81.659 | 1.00 | 56.46 |
| 3381 | CD | ARG | B | 420 | 40.184 | 78.915 | 82.102 | 1.00 | 62.86 |
| 3382 | NE | ARG | B | 420 | 41.274 | 79.587 | 81.235 | 1.00 | 64.61 |
| 3383 | CZ | ARG | B | 420 | 42.623 | 79.785 | 81.598 | 1.00 | 72.93 |
| 3384 | NH1 | ARG | B | 420 | 43.135 | 79.398 | 82.846 | 1.00 | 69.01 |
| 3385 | NH2 | ARG | B | 420 | 43.371 | 80.432 | 80.745 | 1.00 | 67.63 |
| 3386 | C | ARG | B | 420 | 37.183 | 75.268 | 80.668 | 1.00 | 39.24 |
| 3387 | O | ARG | B | 420 | 35.978 | 75.681 | 80.562 | 1.00 | 36.26 |
| 3388 | N | MET | B | 421 | 37.861 | 74.665 | 79.714 | 1.00 | 38.39 |
| 3389 | CA | MET | B | 421 | 37.144 | 74.346 | 78.420 | 1.00 | 37.04 |
| 3390 | CB | MET | B | 421 | 37.987 | 73.658 | 77.523 | 1.00 | 41.19 |
| 3391 | CG | MET | B | 421 | 37.969 | 73.936 | 76.045 | 1.00 | 45.40 |
| 3392 | SD | MET | B | 421 | 39.536 | 73.304 | 75.190 | 1.00 | 51.40 |
| 3393 | CE | MET | B | 421 | 40.851 | 74.414 | 75.729 | 1.00 | 49.13 |
| 3394 | C | MET | B | 421 | 35.978 | 73.351 | 78.829 | 1.00 | 37.69 |
| 3395 | O | MET | B | 421 | 34.903 | 73.407 | 78.309 | 1.00 | 32.26 |
| 3396 | N | LEU | B | 422 | 36.355 | 72.416 | 79.710 | 1.00 | 36.06 |
| 3397 | CA | LEU | B | 422 | 35.330 | 71.374 | 79.968 | 1.00 | 35.29 |
| 3398 | CB | LEU | B | 422 | 36.000 | 70.198 | 80.904 | 1.00 | 36.87 |
| 3399 | CG | LEU | B | 422 | 36.653 | 69.113 | 80.172 | 1.00 | 35.04 |
| 3400 | CD1 | LEU | B | 422 | 36.781 | 67.981 | 81.004 | 1.00 | 33.56 |
| 3401 | CD2 | LEU | B | 422 | 35.908 | 68.687 | 78.963 | 1.00 | 36.81 |
| 3402 | C | LEU | B | 422 | 34.253 | 71.997 | 80.830 | 1.00 | 33.87 |
| 3403 | O | LEU | B | 422 | 33.061 | 71.535 | 80.667 | 1.00 | 33.92 |
| 3404 | N | MET | B | 423 | 34.585 | 72.912 | 81.650 | 1.00 | 29.57 |
| 3405 | CA | MET | B | 423 | 33.600 | 73.624 | 82.403 | 1.00 | 32.22 |
| 3406 | CB | MET | B | 423 | 34.294 | 74.536 | 83.477 | 1.00 | 31.57 |
| 3407 | CG | MET | B | 423 | 34.735 | 73.357 | 84.683 | 1.00 | 35.36 |
| 3408 | SD | MET | B | 423 | 35.107 | 74.163 | 86.203 | 1.00 | 53.40 |
| 3409 | CE | MET | B | 423 | 36.696 | 74.684 | 85.904 | 1.00 | 64.75 |
| 3410 | C | MET | B | 423 | 32.629 | 74.440 | 81.627 | 1.00 | 35.18 |
| 3411 | O | MET | B | 423 | 31.546 | 74.797 | 82.080 | 1.00 | 33.67 |
| 3412 | N | LYS | B | 424 | 32.978 | 74.679 | 80.330 | 1.00 | 31.98 |
| 3413 | CA | LYS | B | 424 | 32.001 | 75.338 | 79.457 | 1.00 | 34.96 |
| 3414 | CB | LYS | B | 424 | 32.566 | 75.736 | 78.047 | 1.00 | 31.64 |
| 3415 | CG | LYS | B | 424 | 33.665 | 76.876 | 78.245 | 1.00 | 33.70 |
| 3416 | CD | LYS | B | 424 | 33.028 | 78.047 | 78.937 | 1.00 | 37.16 |
| 3417 | CE | LYS | B | 424 | 33.860 | 79.098 | 79.095 | 1.00 | 51.16 |
| 3418 | NZ | LYS | B | 424 | 33.356 | 80.056 | 80.511 | 1.00 | 56.80 |
| 3419 | C | LYS | B | 424 | 30.799 | 74.347 | 79.172 | 1.00 | 27.90 |
| 3420 | O | LYS | B | 424 | 29.708 | 74.759 | 78.800 | 1.00 | 32.10 |
| 3421 | N | LEU | B | 425 | 31.088 | 73.093 | 79.416 | 1.00 | 26.30 |
| 3422 | CA | LEU | B | 425 | 29.951 | 72.098 | 79.182 | 1.00 | 27.36 |
| 3423 | CB | LEU | B | 425 | 30.568 | 70.643 | 79.277 | 1.00 | 29.69 |
| 3424 | CG | LEU | B | 425 | 31.624 | 70.197 | 78.046 | 1.00 | 33.03 |
| 3425 | CD1 | LEU | B | 425 | 32.018 | 68.907 | 78.317 | 1.00 | 27.54 |
| 3426 | CD2 | LEU | B | 425 | 30.744 | 70.186 | 76.580 | 1.00 | 28.56 |
| 3427 | C | LEU | B | 425 | 29.064 | 72.274 | 80.333 | 1.00 | 30.48 |
| 3428 | O | LEU | B | 425 | 27.778 | 72.085 | 80.229 | 1.00 | 28.12 |
| 3429 | N | VAL | B | 426 | 29.611 | 72.729 | 81.514 | 1.00 | 25.81 |
| 3430 | CA | VAL | B | 426 | 28.680 | 73.006 | 82.672 | 1.00 | 27.78 |
| 3431 | CB | VAL | B | 426 | 29.545 | 73.358 | 84.058 | 1.00 | 22.37 |
| 3432 | CG1 | VAL | B | 426 | 28.573 | 73.585 | 85.124 | 1.00 | 22.01 |
| 3433 | CG2 | VAL | B | 426 | 30.318 | 72.211 | 84.478 | 1.00 | 22.98 |
| 3434 | C | VAL | B | 426 | 27.886 | 74.163 | 82.329 | 1.00 | 28.34 |
| 3435 | O | VAL | B | 426 | 26.653 | 74.131 | 82.586 | 1.00 | 28.22 |
| 3436 | N | SER | B | 427 | 28.478 | 75.219 | 81.832 | 1.00 | 25.84 |
| 3437 | CA | SER | B | 427 | 27.549 | 76.284 | 81.534 | 1.00 | 32.96 |
| 3438 | CB | SER | B | 427 | 28.249 | 77.696 | 81.257 | 1.00 | 27.16 |
| 3439 | OG | SER | B | 427 | 29.452 | 77.559 | 80.757 | 1.00 | 46.67 |
| 3440 | C | SER | B | 427 | 26.494 | 76.012 | 80.378 | 1.00 | 32.01 |
| 3441 | O | SER | B | 427 | 25.426 | 76.561 | 80.366 | 1.00 | 28.93 |
| 3442 | N | LEU | B | 428 | 26.952 | 75.178 | 79.412 | 1.00 | 32.02 |
| 3443 | CA | LEU | B | 428 | 25.988 | 74.865 | 78.278 | 1.00 | 32.40 |
| 3444 | CB | LEU | B | 428 | 26.717 | 73.884 | 77.281 | 1.00 | 27.32 |
| 3445 | CG | LEU | B | 428 | 27.761 | 74.628 | 76.300 | 1.00 | 39.39 |
| 3446 | CD1 | LEU | B | 428 | 28.596 | 73.661 | 75.609 | 1.00 | 36.25 |
| 3447 | CD2 | LEU | B | 428 | 26.946 | 75.485 | 75.166 | 1.00 | 33.44 |
| 3448 | C | LEU | B | 428 | 24.795 | 74.142 | 78.896 | 1.00 | 31.04 |
| 3449 | O | LEU | B | 428 | 23.657 | 74.287 | 78.397 | 1.00 | 35.47 |
| 3450 | N | ARG | B | 429 | 24.965 | 73.285 | 79.880 | 1.00 | 32.04 |
| 3451 | CA | ARG | B | 429 | 23.903 | 72.579 | 80.461 | 1.00 | 32.80 |
| 3452 | CB | ARG | B | 429 | 24.442 | 71.546 | 81.475 | 1.00 | 30.73 |
| 3453 | CG | ARG | B | 429 | 23.264 | 70.488 | 81.922 | 1.00 | 40.21 |
| 3454 | CD | ARG | B | 429 | 23.022 | 69.498 | 80.606 | 1.00 | 33.81 |
| 3455 | NE | ARG | B | 429 | 24.243 | 68.638 | 80.318 | 1.00 | 28.60 |
| 3456 | CZ | ARG | B | 429 | 24.368 | 67.415 | 80.882 | 1.00 | 32.87 |
| 3457 | NH1 | ARG | B | 429 | 23.505 | 67.018 | 81.810 | 1.00 | 30.23 |
| 3458 | NH2 | ARG | B | 429 | 25.267 | 66.574 | 80.457 | 1.00 | 32.14 |
| 3459 | C | ARG | B | 429 | 22.813 | 73.454 | 80.953 | 1.00 | 33.59 |
| 3460 | O | ARG | B | 429 | 21.657 | 73.192 | 80.936 | 1.00 | 37.09 |
| 3461 | N | THR | B | 430 | 23.201 | 74.544 | 81.614 | 1.00 | 38.72 |
| 3462 | CA | THR | B | 430 | 22.306 | 75.483 | 82.243 | 1.00 | 37.01 |
| 3463 | CB | THR | B | 430 | 23.120 | 76.468 | 83.049 | 1.00 | 34.12 |
| 3464 | OG1 | THR | B | 430 | 23.513 | 75.732 | 84.268 | 1.00 | 38.65 |
| 3465 | CG2 | THR | B | 430 | 22.217 | 77.724 | 83.460 | 1.00 | 32.93 |
| 3466 | C | THR | B | 430 | 21.592 | 76.362 | 80.972 | 1.00 | 39.46 |
| 3467 | O | THR | B | 430 | 20.447 | 76.527 | 81.004 | 1.00 | 35.20 |
| 3468 | N | LEU | B | 431 | 22.416 | 76.778 | 80.127 | 1.00 | 36.01 |
| 3469 | CA | LEU | B | 431 | 21.991 | 77.479 | 78.958 | 1.00 | 42.02 |
| 3470 | CB | LEU | B | 431 | 23.139 | 77.674 | 78.016 | 1.00 | 31.12 |
| 3471 | CG | LEU | B | 431 | 24.016 | 78.912 | 78.364 | 1.00 | 35.43 |
| 3472 | CD1 | LEU | B | 431 | 25.168 | 78.964 | 77.609 | 1.00 | 34.81 |
| 3473 | CD2 | LEU | B | 431 | 23.470 | 80.210 | 78.169 | 1.00 | 37.53 |
| 3474 | C | LEU | B | 431 | 20.850 | 76.641 | 78.326 | 1.00 | 43.70 |
| 3475 | O | LEU | B | 431 | 19.839 | 77.114 | 78.022 | 1.00 | 44.09 |
| 3476 | N | SER | B | 432 | 21.079 | 75.379 | 78.252 | 1.00 | 46.54 |
| 3477 | CA | SER | B | 432 | 20.086 | 74.518 | 77.706 | 1.00 | 49.15 |
| 3478 | CB | SER | B | 432 | 20.435 | 73.053 | 77.806 | 1.00 | 48.94 |
| 3479 | OG | SER | B | 432 | 19.413 | 72.247 | 77.466 | 1.00 | 45.95 |
| 3480 | C | SER | B | 432 | 18.809 | 74.754 | 78.317 | 1.00 | 49.69 |
| 3481 | O | SER | B | 432 | 17.776 | 74.784 | 77.516 | 1.00 | 54.26 |
| 3482 | N | SER | B | 433 | 18.724 | 74.768 | 79.580 | 1.00 | 47.14 |
| 3483 | CA | SER | B | 433 | 17.494 | 74.980 | 80.296 | 1.00 | 47.20 |
| 3484 | CB | SER | B | 433 | 17.739 | 74.865 | 81.794 | 1.00 | 40.62 |
| 3485 | OG | SER | B | 433 | 18.081 | 73.584 | 82.108 | 1.00 | 49.73 |
| 3486 | C | SER | B | 433 | 16.946 | 76.447 | 80.127 | 1.00 | 49.50 |
| 3487 | O | SER | B | 433 | 15.794 | 76.592 | 80.321 | 1.00 | 51.14 |
| 3488 | N | VAL | B | 434 | 17.789 | 77.435 | 79.846 | 1.00 | 49.22 |
| 3489 | CA | VAL | B | 434 | 17.229 | 78.708 | 79.746 | 1.00 | 54.55 |
| 3490 | CB | VAL | B | 434 | 18.304 | 79.836 | 79.960 | 1.00 | 54.92 |
| 3491 | CG1 | VAL | B | 434 | 18.835 | 79.844 | 81.638 | 1.00 | 50.26 |
| 3492 | CG2 | VAL | B | 434 | 19.391 | 79.488 | 79.048 | 1.00 | 57.15 |
| 3493 | C | VAL | B | 434 | 16.649 | 78.814 | 78.329 | 1.00 | 51.87 |
| 3494 | O | VAL | B | 434 | 15.982 | 79.691 | 78.010 | 1.00 | 51.61 |
| 3495 | N | HIS | B | 435 | 17.105 | 78.026 | 77.512 | 1.00 | 50.38 |
| 3496 | CA | HIS | B | 435 | 16.629 | 77.927 | 76.158 | 1.00 | 55.58 |
| 3497 | CB | HIS | B | 435 | 17.473 | 77.180 | 75.326 | 1.00 | 56.02 |
| 3498 | CG | HIS | B | 435 | 16.897 | 76.877 | 74.054 | 1.00 | 69.93 |
| 3499 | ND1 | HIS | B | 435 | 17.388 | 77.444 | 72.810 | 1.00 | 81.64 |
| 3500 | CE1 | HIS | B | 435 | 16.679 | 76.915 | 71.782 | 1.00 | 69.58 |
| 3501 | NE2 | HIS | B | 435 | 15.850 | 75.956 | 72.306 | 1.00 | 62.02 |
| 3502 | CD2 | HIS | B | 435 | 16.030 | 75.888 | 73.694 | 1.00 | 64.25 |
| 3503 | C | HIS | B | 435 | 15.424 | 77.348 | 76.187 | 1.00 | 59.49 |
| 3504 | O | HIS | B | 435 | 14.621 | 77.689 | 75.624 | 1.00 | 63.73 |
| 3505 | N | SER | B | 436 | 15.158 | 76.559 | 77.155 | 1.00 | 62.97 |
| 3506 | CA | SER | B | 436 | 13.838 | 75.852 | 77.250 | 1.00 | 61.17 |
| 3507 | CB | SER | B | 436 | 14.017 | 74.539 | 77.996 | 1.00 | 59.04 |
| 3508 | OG | SER | B | 436 | 12.958 | 74.100 | 78.611 | 1.00 | 57.03 |
| 3509 | C | SER | B | 436 | 12.781 | 76.645 | 77.922 | 1.00 | 64.10 |
| 3510 | O | SER | B | 436 | 11.687 | 76.568 | 77.568 | 1.00 | 69.87 |
| 3511 | N | GLU | B | 437 | 12.941 | 77.315 | 78.991 | 1.00 | 65.48 |
| 3512 | CA | GLU | B | 437 | 11.928 | 78.060 | 79.623 | 1.00 | 68.26 |
| 3513 | CB | GLU | B | 437 | 12.252 | 78.369 | 81.135 | 1.00 | 69.08 |
| 3514 | CG | GLU | B | 437 | 13.770 | 78.578 | 81.471 | 1.00 | 77.67 |
| 3515 | CD | GLU | B | 437 | 13.995 | 78.877 | 82.975 | 1.00 | 84.13 |
| 3516 | OE1 | GLU | B | 437 | 12.918 | 79.204 | 83.662 | 1.00 | 96.00 |
| 3517 | OE2 | GLU | B | 437 | 15.114 | 78.770 | 83.468 | 1.00 | 88.44 |
| 3518 | C | GLU | B | 437 | 11.735 | 79.431 | 78.733 | 1.00 | 69.36 |
| 3519 | O | GLU | B | 437 | 10.776 | 80.203 | 78.843 | 1.00 | 74.35 |
| 3520 | N | GLN | B | 438 | 12.603 | 79.707 | 77.908 | 1.00 | 63.83 |
| 3521 | CA | GLN | B | 438 | 12.404 | 80.735 | 77.024 | 1.00 | 61.73 |
| 3522 | CB | GLN | B | 438 | 13.664 | 81.035 | 76.242 | 1.00 | 54.68 |
| 3523 | CG | GLN | B | 438 | 13.751 | 81.379 | 74.980 | 1.00 | 53.55 |
| 3524 | CD | GLN | B | 438 | 13.375 | 83.010 | 74.543 | 1.00 | 64.01 |
| 3525 | OE1 | GLN | B | 438 | 12.462 | 83.552 | 75.147 | 1.00 | 55.61 |
| 3526 | NE2 | GLN | B | 438 | 14.163 | 83.611 | 73.698 | 1.00 | 54.83 |
| 3527 | C | GLN | B | 438 | 11.392 | 80.405 | 76.119 | 1.00 | 71.21 |
| 3528 | O | GLN | B | 438 | 10.254 | 81.176 | 75.834 | 1.00 | 77.04 |
| 3529 | N | VAL | B | 439 | 11.707 | 79.347 | 75.472 | 1.00 | 73.56 |
| 3530 | CA | VAL | B | 439 | 10.781 | 78.826 | 74.528 | 1.00 | 77.85 |
| 3531 | CB | VAL | B | 439 | 11.370 | 77.428 | 73.962 | 1.00 | 77.61 |
| 3532 | CG1 | VAL | B | 439 | 10.095 | 76.647 | 73.195 | 1.00 | 83.57 |
| 3533 | CG2 | VAL | B | 439 | 12.539 | 77.639 | 73.039 | 1.00 | 72.22 |
| 3534 | C | VAL | B | 439 | 9.319 | 78.686 | 75.069 | 1.00 | 76.34 |
| 3535 | O | VAL | B | 439 | 8.341 | 78.881 | 74.376 | 1.00 | 74.98 |
| 3536 | N | PHE | B | 440 | 9.206 | 78.273 | 76.317 | 1.00 | 79.29 |
| 3537 | CA | PHE | B | 440 | 7.996 | 78.110 | 76.985 | 1.00 | 80.16 |
| 3538 | CB | PHE | B | 440 | 8.225 | 77.599 | 78.238 | 1.00 | 78.89 |
| 3539 | CG | PHE | B | 440 | 7.054 | 77.724 | 79.098 | 1.00 | 92.23 |
| 3540 | CD1 | PHE | B | 440 | 5.772 | 77.208 | 78.742 | 1.00 | 99.43 |
| 3541 | CE1 | PHE | B | 440 | 4.596 | 77.379 | 79.606 | 1.00 | 98.72 |
| 3542 | CZ | PHE | B | 440 | 4.740 | 78.132 | 80.830 | 1.00 | 100.73 |
| 3543 | CE2 | PHE | B | 440 | 5.999 | 78.723 | 81.122 | 1.00 | 99.88 |
| 3544 | CD2 | PHE | B | 440 | 7.139 | 78.412 | 80.292 | 1.00 | 101.24 |
| 3545 | C | PHE | B | 440 | 7.379 | 79.541 | 77.198 | 1.00 | 85.20 |
| 3546 | O | PHE | B | 440 | 6.111 | 79.770 | 77.380 | 1.00 | 84.97 |
| 3547 | N | ALA | B | 441 | 8.196 | 80.588 | 77.096 | 1.00 | 86.29 |
| 3548 | CA | ALA | B | 441 | 7.624 | 81.976 | 77.265 | 1.00 | 80.96 |
| 3549 | CB | ALA | B | 441 | 8.536 | 82.970 | 77.776 | 1.00 | 74.47 |
| 3550 | C | ALA | B | 441 | 7.220 | 82.419 | 75.924 | 1.00 | 80.06 |
| 3551 | O | ALA | B | 441 | 6.132 | 83.160 | 75.872 | 1.00 | 81.05 |
| 3552 | N | LEU | B | 442 | 8.025 | 82.135 | 74.899 | 1.00 | 78.80 |
| 3553 | CA | LEU | B | 442 | 7.568 | 82.409 | 73.552 | 1.00 | 82.11 |
| 3554 | CB | LEU | B | 442 | 8.443 | 81.895 | 72.505 | 1.00 | 81.69 |
| 3555 | CG | LEU | B | 442 | 9.624 | 82.735 | 72.473 | 1.00 | 83.56 |
| 3556 | CD1 | LEU | B | 442 | 10.612 | 82.111 | 71.506 | 1.00 | 80.19 |
| 3557 | CD2 | LEU | B | 442 | 9.248 | 84.129 | 72.041 | 1.00 | 77.57 |
| 3558 | C | LEU | B | 442 | 6.020 | 81.763 | 73.158 | 1.00 | 85.14 |
| 3559 | O | LEU | B | 442 | 5.034 | 82.354 | 72.611 | 1.00 | 85.70 |
| 3560 | N | ARG | B | 443 | 5.815 | 80.602 | 73.482 | 1.00 | 85.53 |
| 3561 | CA | ARG | B | 443 | 4.545 | 80.084 | 73.343 | 1.00 | 87.83 |
| 3562 | CB | ARG | B | 443 | 4.619 | 78.683 | 73.798 | 1.00 | 91.21 |
| 3563 | CG | ARG | B | 443 | 3.342 | 77.748 | 73.912 | 1.00 | 98.16 |
| 3564 | CD | ARG | B | 443 | 3.700 | 76.185 | 73.899 | 1.00 | 92.87 |
| 3565 | NE | ARG | B | 443 | 4.850 | 75.964 | 72.925 | 1.00 | 93.04 |
| 3566 | CZ | ARG | B | 443 | 4.869 | 76.230 | 71.575 | 1.00 | 96.00 |
| 3567 | NH1 | ARG | B | 443 | 3.705 | 76.661 | 70.926 | 1.00 | 102.88 |
| 3568 | NH2 | ARG | B | 443 | 5.990 | 75.956 | 70.841 | 1.00 | 82.77 |
| 3569 | C | ARG | B | 443 | 3.639 | 80.845 | 74.249 | 1.00 | 90.37 |
| 3570 | O | ARG | B | 443 | 2.454 | 81.127 | 73.835 | 1.00 | 92.49 |
| 3571 | N | ALA | B | 444 | 4.064 | 81.126 | 75.507 | 1.00 | 93.02 |
| 3572 | CA | ALA | B | 444 | 3.244 | 81.840 | 76.538 | 1.00 | 96.93 |
| 3573 | CB | ALA | B | 444 | 3.975 | 82.137 | 77.768 | 1.00 | 95.28 |
| 3574 | C | ALA | B | 444 | 2.606 | 83.229 | 75.946 | 1.00 | 102.42 |
| 3575 | O | ALA | B | 444 | 1.426 | 83.696 | 76.300 | 1.00 | 101.79 |
| 3576 | N | GLN | B | 445 | 3.350 | 83.862 | 75.003 | 1.00 | 102.89 |
| 3577 | CA | GLN | B | 445 | 2.908 | 85.111 | 74.382 | 1.00 | 101.00 |
| 3578 | CB | GLN | B | 445 | 4.009 | 86.219 | 74.353 | 1.00 | 100.25 |
| 3579 | CG | GLN | B | 445 | 3.857 | 87.230 | 75.518 | 1.00 | 99.82 |
| 3580 | CD | GLN | B | 445 | 2.569 | 88.091 | 75.407 | 1.00 | 104.75 |
| 3581 | OE1 | GLN | B | 445 | 1.973 | 88.471 | 76.447 | 1.00 | 101.58 |
| 3582 | NE2 | GLN | B | 445 | 2.156 | 88.408 | 74.153 | 1.00 | 101.48 |
| 3583 | C | GLN | B | 445 | 2.285 | 84.948 | 73.003 | 1.00 | 101.79 |
| 3584 | O | GLN | B | 445 | 1.020 | 85.221 | 72.835 | 1.00 | 107.41 |
| 3585 | N | ASP | B | 446 | 3.080 | 84.594 | 72.015 | 1.00 | 98.12 |
| 3586 | CA | ASP | B | 446 | 2.583 | 84.498 | 70.639 | 1.00 | 97.10 |
| 3587 | CB | ASP | B | 446 | 3.552 | 85.344 | 69.702 | 1.00 | 95.60 |
| 3588 | CG | ASP | B | 446 | 3.395 | 86.855 | 69.975 | 1.00 | 106.20 |
| 3589 | OD1 | ASP | B | 446 | 2.221 | 87.369 | 70.349 | 1.00 | 105.08 |
| 3590 | OD2 | ASP | B | 446 | 4.448 | 87.602 | 69.878 | 1.00 | 103.97 |
| 3591 | C | ASP | B | 446 | 2.492 | 83.188 | 70.052 | 1.00 | 90.74 |
| 3592 | O | ASP | B | 446 | 1.567 | 82.679 | 69.995 | 1.00 | 91.91 |
| 3593 | N | LYS | B | 447 | 3.603 | 82.850 | 69.535 | 1.00 | 91.18 |
| 3594 | CA | LYS | B | 447 | 3.986 | 81.746 | 68.713 | 1.00 | 92.43 |
| 3595 | CB | LYS | B | 447 | 4.987 | 82.291 | 67.750 | 1.00 | 92.71 |
| 3596 | CG | LYS | B | 447 | 5.694 | 83.445 | 68.230 | 1.00 | 98.18 |
| 3597 | CD | LYS | B | 447 | 6.175 | 83.217 | 69.631 | 1.00 | 93.65 |
| 3598 | CE | LYS | B | 447 | 6.434 | 84.685 | 70.142 | 1.00 | 117.01 |
| 3599 | NZ | LYS | B | 447 | 5.874 | 85.264 | 71.584 | 1.00 | 106.41 |
| 3600 | C | LYS | B | 447 | 4.554 | 80.409 | 69.258 | 1.00 | 90.83 |
| 3601 | O | LYS | B | 447 | 5.379 | 80.381 | 70.262 | 1.00 | 90.09 |
| 3602 | N | LYS | B | 448 | 4.126 | 79.366 | 68.489 | 1.00 | 88.27 |
| 3603 | CA | LYS | B | 448 | 4.415 | 78.029 | 68.617 | 1.00 | 82.99 |
| 3604 | CB | LYS | B | 448 | 3.292 | 77.102 | 68.031 | 1.00 | 88.96 |
| 3605 | CG | LYS | B | 448 | 1.812 | 77.531 | 68.035 | 1.00 | 85.79 |
| 3606 | CD | LYS | B | 448 | 1.034 | 76.813 | 67.089 | 1.00 | 87.76 |
| 3607 | CE | LYS | B | 448 | −0.305 | 77.719 | 66.689 | 1.00 | 92.06 |
| 3608 | NZ | LYS | B | 448 | −1.490 | 76.757 | 66.440 | 1.00 | 92.58 |
| 3609 | C | LYS | B | 448 | 5.674 | 77.916 | 67.825 | 1.00 | 84.11 |
| 3610 | O | LYS | B | 448 | 6.166 | 78.859 | 67.367 | 1.00 | 82.85 |
| 3611 | N | LEU | B | 449 | 6.304 | 76.694 | 67.799 | 1.00 | 87.83 |
| 3612 | CA | LEU | B | 449 | 7.532 | 76.347 | 67.134 | 1.00 | 80.67 |
| 3613 | CB | LEU | B | 449 | 8.129 | 75.591 | 68.224 | 1.00 | 80.57 |
| 3614 | CG | LEU | B | 449 | 8.368 | 76.165 | 69.573 | 1.00 | 78.58 |
| 3615 | CD1 | LEU | B | 449 | 8.768 | 74.939 | 70.442 | 1.00 | 82.07 |
| 3616 | CD2 | LEU | B | 449 | 9.688 | 77.084 | 69.684 | 1.00 | 74.82 |
| 3617 | C | LEU | B | 449 | 7.267 | 75.353 | 65.939 | 1.00 | 77.71 |
| 3618 | O | LEU | B | 449 | 6.382 | 74.513 | 66.111 | 1.00 | 77.67 |
| 3619 | N | PRO | B | 450 | 8.133 | 75.376 | 64.934 | 1.00 | 71.21 |
| 3620 | CA | PRO | B | 450 | 8.214 | 74.509 | 63.793 | 1.00 | 73.98 |
| 3621 | CB | PRO | B | 450 | 9.454 | 74.915 | 63.105 | 1.00 | 68.98 |
| 3622 | CG | PRO | B | 450 | 9.464 | 76.294 | 63.381 | 1.00 | 67.90 |
| 3623 | CD | PRO | B | 450 | 9.142 | 76.345 | 64.922 | 1.00 | 69.62 |
| 3624 | C | PRO | B | 450 | 8.135 | 73.055 | 64.176 | 1.00 | 77.78 |
| 3625 | O | PRO | B | 450 | 8.994 | 72.666 | 64.840 | 1.00 | 83.46 |
| 3626 | N | PRO | B | 451 | 7.029 | 72.292 | 63.742 | 1.00 | 79.07 |
| 3627 | CA | PRO | B | 451 | 6.995 | 70.864 | 64.051 | 1.00 | 72.39 |
| 3628 | CB | PRO | B | 451 | 6.181 | 70.317 | 62.928 | 1.00 | 72.77 |
| 3629 | CG | PRO | B | 451 | 5.066 | 71.463 | 62.706 | 1.00 | 80.55 |
| 3630 | CD | PRO | B | 451 | 6.059 | 72.638 | 62.734 | 1.00 | 73.48 |
| 3631 | C | PRO | B | 451 | 8.377 | 70.203 | 64.101 | 1.00 | 69.63 |
| 3632 | O | PRO | B | 451 | 8.447 | 69.326 | 64.847 | 1.00 | 74.53 |
| 3633 | N | LEU | B | 452 | 9.267 | 70.673 | 63.327 | 1.00 | 67.85 |
| 3634 | CA | LEU | B | 452 | 10.622 | 70.096 | 63.322 | 1.00 | 68.90 |
| 3635 | CB | LEU | B | 452 | 11.275 | 70.644 | 62.138 | 1.00 | 63.66 |
| 3636 | CG | LEU | B | 452 | 12.609 | 69.903 | 62.065 | 1.00 | 67.29 |
| 3637 | CD1 | LEU | B | 452 | 12.666 | 68.400 | 62.235 | 1.00 | 78.79 |
| 3638 | CD2 | LEU | B | 452 | 13.805 | 70.222 | 60.707 | 1.00 | 64.79 |
| 3639 | C | LEU | B | 452 | 11.498 | 70.348 | 64.590 | 1.00 | 76.88 |
| 3640 | O | LEU | B | 452 | 12.329 | 69.590 | 65.011 | 1.00 | 76.42 |
| 3641 | N | LEU | B | 453 | 11.215 | 71.485 | 65.226 | 1.00 | 75.46 |
| 3642 | CA | LEU | B | 453 | 11.931 | 71.872 | 66.324 | 1.00 | 74.07 |
| 3643 | CB | LEU | B | 453 | 12.344 | 73.481 | 66.225 | 1.00 | 76.58 |
| 3644 | CG | LEU | B | 453 | 13.177 | 73.917 | 65.021 | 1.00 | 73.28 |
| 3645 | CD1 | LEU | B | 453 | 13.359 | 75.377 | 64.905 | 1.00 | 84.76 |
| 3646 | CD2 | LEU | B | 453 | 14.370 | 73.304 | 65.144 | 1.00 | 83.41 |
| 3647 | C | LEU | B | 453 | 10.997 | 71.555 | 67.373 | 1.00 | 72.22 |
| 3648 | O | LEU | B | 453 | 11.406 | 71.504 | 68.556 | 1.00 | 73.02 |
| 3649 | N | SER | B | 454 | 9.726 | 71.580 | 67.055 | 1.00 | 70.01 |
| 3650 | CA | SER | B | 454 | 8.763 | 71.270 | 68.090 | 1.00 | 73.57 |
| 3651 | CB | SER | B | 454 | 7.450 | 71.287 | 67.508 | 1.00 | 71.14 |
| 3652 | OG | SER | B | 454 | 6.551 | 70.409 | 68.142 | 1.00 | 74.80 |
| 3653 | C | SER | B | 454 | 9.104 | 69.817 | 68.646 | 1.00 | 78.17 |
| 3654 | O | SER | B | 454 | 8.730 | 69.476 | 69.704 | 1.00 | 73.62 |
| 3655 | N | GLU | B | 455 | 9.847 | 69.020 | 67.786 | 1.00 | 82.20 |
| 3656 | CA | GLU | B | 455 | 10.159 | 67.663 | 68.045 | 1.00 | 84.44 |
| 3657 | CB | GLU | B | 455 | 10.787 | 66.996 | 66.773 | 1.00 | 89.19 |
| 3658 | CG | GLU | B | 455 | 10.166 | 65.593 | 66.486 | 1.00 | 96.86 |
| 3659 | CD | GLU | B | 455 | 8.492 | 65.711 | 66.504 | 1.00 | 115.60 |
| 3660 | OE1 | GLU | B | 455 | 7.775 | 66.633 | 65.744 | 1.00 | 117.40 |
| 3661 | OE2 | GLU | B | 455 | 7.931 | 64.946 | 67.394 | 1.00 | 106.60 |
| 3662 | C | GLU | B | 455 | 11.219 | 67.481 | 69.107 | 1.00 | 80.43 |
| 3663 | O | GLU | B | 455 | 11.181 | 66.527 | 69.876 | 1.00 | 76.11 |
| 3664 | N | ILE | B | 456 | 12.137 | 68.411 | 69.062 | 1.00 | 75.65 |
| 3665 | CA | ILE | B | 456 | 13.198 | 68.334 | 70.069 | 1.00 | 80.83 |
| 3666 | CB | ILE | B | 456 | 14.488 | 68.753 | 69.504 | 1.00 | 78.83 |
| 3667 | CG1 | ILE | B | 456 | 14.135 | 68.610 | 67.952 | 1.00 | 73.26 |
| 3668 | CD1 | ILE | B | 456 | 15.216 | 69.380 | 67.297 | 1.00 | 77.02 |
| 3669 | CG2 | ILE | B | 456 | 15.625 | 67.777 | 69.854 | 1.00 | 78.31 |
| 3670 | C | ILE | B | 456 | 13.115 | 69.203 | 71.185 | 1.00 | 76.42 |
| 3671 | O | ILE | B | 456 | 13.565 | 68.806 | 72.158 | 1.00 | 79.20 |
| 3672 | N | TRP | B | 457 | 12.365 | 70.242 | 71.086 | 1.00 | 71.39 |
| 3673 | CA | TRP | B | 457 | 12.292 | 71.151 | 72.176 | 1.00 | 76.38 |
| 3674 | CB | TRP | B | 457 | 12.896 | 72.533 | 71.803 | 1.00 | 68.20 |
| 3675 | CG | TRP | B | 457 | 14.232 | 72.476 | 71.499 | 1.00 | 68.30 |
| 3676 | CD1 | TRP | B | 457 | 15.126 | 71.846 | 72.231 | 1.00 | 57.85 |
| 3677 | NE1 | TRP | B | 457 | 16.342 | 71.998 | 71.601 | 1.00 | 64.57 |
| 3678 | CE2 | TRP | B | 457 | 16.274 | 72.814 | 70.549 | 1.00 | 61.33 |
| 3679 | CD2 | TRP | B | 457 | 14.970 | 73.180 | 70.458 | 1.00 | 58.63 |
| 3680 | CE3 | TRP | B | 457 | 14.636 | 74.007 | 69.475 | 1.00 | 68.32 |
| 3681 | CZ3 | TRP | B | 457 | 15.566 | 74.400 | 68.431 | 1.00 | 53.89 |
| 3682 | CH2 | TRP | B | 457 | 16.898 | 74.074 | 68.568 | 1.00 | 59.04 |
| 3683 | CZ2 | TRP | B | 457 | 17.320 | 73.320 | 69.692 | 1.00 | 60.47 |
| 3684 | C | TRP | B | 457 | 11.004 | 71.380 | 73.030 | 1.00 | 79.49 |
| 3685 | O | TRP | B | 457 | 11.014 | 71.998 | 74.102 | 1.00 | 84.07 |
| 3686 | N | ASP | B | 458 | 9.909 | 70.953 | 72.589 | 1.00 | 81.55 |
| 3687 | CA | ASP | B | 458 | 8.792 | 71.192 | 73.405 | 1.00 | 88.84 |
| 3688 | CB | ASP | B | 458 | 7.559 | 71.444 | 72.515 | 1.00 | 91.13 |
| 3689 | CG | ASP | B | 458 | 6.894 | 72.822 | 72.763 | 1.00 | 93.15 |
| 3690 | OD1 | ASP | B | 458 | 6.473 | 73.295 | 71.709 | 1.00 | 89.76 |
| 3691 | OD2 | ASP | B | 458 | 6.706 | 73.413 | 73.940 | 1.00 | 80.41 |
| 3692 | C | ASP | B | 458 | 8.384 | 70.242 | 74.504 | 1.00 | 94.89 |
| 3693 | O | ASP | B | 458 | 8.794 | 70.470 | 75.790 | 1.00 | 103.33 |
| 3694 | N | VAL | B | 459 | 7.573 | 69.231 | 74.181 | 1.00 | 94.94 |
| 3695 | CA | VAL | B | 459 | 7.086 | 68.485 | 75.295 | 1.00 | 101.47 |
| 3696 | CB | VAL | B | 459 | 5.754 | 69.037 | 75.851 | 1.00 | 99.71 |
| 3697 | CG1 | VAL | B | 459 | 5.476 | 68.479 | 77.239 | 1.00 | 105.10 |
| 3698 | CG2 | VAL | B | 459 | 5.911 | 70.574 | 76.027 | 1.00 | 109.26 |
| 3699 | C | VAL | B | 459 | 7.116 | 66.947 | 75.396 | 1.00 | 104.18 |
| 3700 | O | VAL | B | 459 | 7.313 | 66.156 | 74.438 | 1.00 | 101.60 |
| 3701 | N | HIS | B | 460 | 6.874 | 66.555 | 76.675 | 1.00 | 107.43 |
| 3702 | CA | HIS | B | 460 | 7.073 | 65.202 | 77.282 | 1.00 | 107.42 |
| 3703 | CB | HIS | B | 460 | 8.072 | 65.379 | 78.452 | 1.00 | 107.83 |
| 3704 | CG | HIS | B | 460 | 9.321 | 64.541 | 78.343 | 1.00 | 106.96 |
| 3705 | ND1 | HIS | B | 460 | 9.320 | 63.161 | 78.038 | 1.00 | 106.22 |
| 3706 | CE1 | HIS | B | 460 | 10.563 | 62.706 | 78.071 | 1.00 | 102.14 |
| 3707 | NE2 | HIS | B | 460 | 11.360 | 63.731 | 78.314 | 1.00 | 105.06 |
| 3708 | CD2 | HIS | B | 460 | 10.609 | 64.889 | 78.537 | 1.00 | 101.46 |
| 3709 | C | HIS | B | 460 | 5.855 | 64.306 | 77.786 | 1.00 | 107.54 |
| 3710 | O | HIS | B | 460 | 5.948 | 63.795 | 78.950 | 1.00 | 102.46 |
| 3711 | OXT | HIS | B | 460 | 4.828 | 64.047 | 77.024 | 1.00 | 111.13 |
| 3712 | F28 | MON | C | 1 | 19.808 | 64.508 | 97.381 | 1.00 | 43.78 |
| 3713 | C23 | MON | C | 1 | 19.416 | 65.568 | 97.788 | 1.00 | 45.54 |
| 3714 | F26 | MON | C | 1 | 20.407 | 66.480 | 97.502 | 1.00 | 50.09 |
| 3715 | F27 | MON | C | 1 | 18.437 | 66.125 | 96.966 | 1.00 | 56.18 |
| 3716 | C19 | MON | C | 1 | 18.942 | 65.415 | 99.375 | 1.00 | 54.41 |
| 3717 | N17 | MON | C | 1 | 18.486 | 66.619 | 99.923 | 1.00 | 52.26 |
| 3718 | C16 | MON | C | 1 | 17.301 | 67.177 | 99.588 | 1.00 | 52.94 |
| 3719 | C9 | MON | C | 1 | 17.046 | 68.586 | 99.514 | 1.00 | 59.65 |
| 3720 | C4 | MON | C | 1 | 15.773 | 69.251 | 99.221 | 1.00 | 44.55 |
| 3721 | C8 | MON | C | 1 | 16.109 | 66.497 | 99.440 | 1.00 | 58.47 |
| 3722 | C3 | MON | C | 1 | 14.797 | 67.221 | 99.189 | 1.00 | 44.43 |
| 3723 | C1 | MON | C | 1 | 14.619 | 68.582 | 99.044 | 1.00 | 39.54 |
| 3724 | C2 | MON | C | 1 | 13.324 | 69.219 | 98.845 | 1.00 | 43.70 |
| 3725 | O7 | MON | C | 1 | 13.595 | 70.614 | 98.325 | 1.00 | 42.48 |
| 3726 | C6 | MON | C | 1 | 12.271 | 68.544 | 97.976 | 1.00 | 41.28 |
| 3727 | F15 | MON | C | 1 | 11.230 | 69.333 | 97.783 | 1.00 | 46.95 |
| 3728 | F14 | MON | C | 1 | 11.833 | 67.406 | 98.331 | 1.00 | 43.11 |
| 3729 | F13 | MON | C | 1 | 12.731 | 68.407 | 96.707 | 1.00 | 43.88 |
| 3730 | C5 | MON | C | 1 | 12.672 | 69.514 | 100.243 | 1.00 | 42.61 |
| 3731 | F12 | MON | C | 1 | 11.527 | 70.162 | 100.099 | 1.00 | 49.55 |
| 3732 | F11 | MON | C | 1 | 12.431 | 68.417 | 100.854 | 1.00 | 45.69 |
| 3733 | F10 | MON | C | 1 | 13.360 | 70.263 | 101.134 | 1.00 | 39.44 |
| 3734 | S18 | MON | C | 1 | 19.334 | 66.990 | 101.578 | 1.00 | 78.47 |
| 3735 | O21 | MON | C | 1 | 18.906 | 68.160 | 102.353 | 1.00 | 74.53 |
| 3736 | O22 | MON | C | 1 | 20.928 | 66.810 | 101.606 | 1.00 | 69.11 |
| 3737 | C20 | MON | C | 1 | 18.676 | 65.896 | 102.715 | 1.00 | 59.07 |
| 3738 | C24 | MON | C | 1 | 19.476 | 64.928 | 103.096 | 1.00 | 64.28 |
| 3739 | C29 | MON | C | 1 | 19.096 | 63.952 | 103.934 | 1.00 | 70.23 |
| 3740 | C31 | MON | C | 1 | 17.745 | 63.902 | 104.213 | 1.00 | 73.30 |
| 3741 | C30 | MON | C | 1 | 16.831 | 64.859 | 103.631 | 1.00 | 67.05 |
| 3742 | C25 | MON | C | 1 | 17.311 | 65.865 | 102.880 | 1.00 | 57.46 |
| 3743 | F28 | MON | D | 1 | 22.651 | 80.942 | 70.764 | 1.00 | 69.44 |
| 3744 | C23 | MON | D | 1 | 21.798 | 80.169 | 70.148 | 1.00 | 57.82 |
| 3745 | F26 | MON | D | 1 | 22.543 | 79.119 | 70.213 | 1.00 | 62.78 |
| 3746 | F27 | MON | D | 1 | 20.755 | 80.001 | 70.992 | 1.00 | 61.86 |
| 3747 | C19 | MON | D | 1 | 21.215 | 80.654 | 68.630 | 1.00 | 75.45 |
| 3748 | N17 | MON | D | 1 | 20.440 | 79.761 | 67.906 | 1.00 | 76.60 |
| 3749 | C16 | MON | D | 1 | 19.149 | 79.599 | 68.328 | 1.00 | 68.06 |
| 3750 | C9 | MON | D | 1 | 18.512 | 78.416 | 68.236 | 1.00 | 67.68 |
| 3751 | C4 | MON | D | 1 | 17.186 | 78.252 | 68.702 | 1.00 | 64.13 |
| 3752 | C8 | MON | D | 1 | 18.382 | 80.560 | 68.704 | 1.00 | 67.84 |
| 3753 | C3 | MON | D | 1 | 17.052 | 80.405 | 69.140 | 1.00 | 71.18 |
| 3754 | C1 | MON | D | 1 | 16.449 | 79.271 | 69.191 | 1.00 | 69.42 |
| 3755 | C2 | MON | D | 1 | 14.956 | 79.086 | 69.415 | 1.00 | 68.85 |
| 3756 | O7 | MON | D | 1 | 14.845 | 77.784 | 69.704 | 1.00 | 73.28 |
| 3757 | C6 | MON | D | 1 | 14.333 | 80.022 | 70.298 | 1.00 | 73.50 |
| 3758 | F15 | MON | D | 1 | 13.033 | 79.709 | 70.767 | 1.00 | 73.67 |
| 3759 | F14 | MON | D | 1 | 14.461 | 81.177 | 69.631 | 1.00 | 75.64 |
| 3760 | F13 | MON | D | 1 | 14.843 | 80.117 | 71.635 | 1.00 | 78.70 |
| 3761 | C5 | MON | D | 1 | 14.026 | 79.078 | 68.126 | 1.00 | 65.40 |
| 3762 | F12 | MON | D | 1 | 12.814 | 78.813 | 68.422 | 1.00 | 56.90 |
| 3763 | F11 | MON | D | 1 | 14.145 | 80.191 | 67.512 | 1.00 | 65.11 |
| 3764 | F10 | MON | D | 1 | 14.594 | 78.322 | 67.116 | 1.00 | 67.88 |
| 3765 | S18 | MON | D | 1 | 20.952 | 79.543 | 66.221 | 1.00 | 91.37 |
| 3766 | O21 | MON | D | 1 | 20.850 | 80.997 | 65.429 | 1.00 | 87.99 |
| 3767 | O22 | MON | D | 1 | 19.809 | 79.087 | 65.442 | 1.00 | 87.36 |
| 3768 | C20 | MON | D | 1 | 22.391 | 78.840 | 65.550 | 1.00 | 83.26 |
| 3769 | C24 | MON | D | 1 | 22.355 | 78.117 | 64.483 | 1.00 | 84.25 |
| 3770 | C29 | MON | D | 1 | 23.485 | 77.423 | 64.112 | 1.00 | 105.04 |
| 3771 | C31 | MON | D | 1 | 24.753 | 77.652 | 64.776 | 1.00 | 98.94 |
| 3772 | C30 | MON | D | 1 | 24.682 | 78.634 | 65.738 | 1.00 | 97.53 |
| 3773 | C25 | MON | D | 1 | 23.523 | 79.120 | 66.188 | 1.00 | 88.16 |
| 3774 | F28 | MON | E | 1 | 18.863 | 67.289 | 66.183 | 0.68 | 73.54 |
| 3775 | C23 | MON | E | 1 | 17.901 | 66.491 | 66.389 | 0.68 | 58.02 |
| 3776 | F26 | MON | E | 1 | 18.133 | 66.060 | 67.479 | 0.68 | 59.55 |
| 3777 | F27 | MON | E | 1 | 16.774 | 67.178 | 66.683 | 0.68 | 68.46 |
| 3778 | C19 | MON | E | 1 | 17.457 | 65.561 | 65.236 | 0.68 | 67.48 |
| 3779 | N17 | MON | E | 1 | 16.984 | 64.432 | 65.323 | 0.68 | 70.21 |
| 3780 | C16 | MON | E | 1 | 17.580 | 63.374 | 64.933 | 0.68 | 62.25 |
| 3781 | C9 | MON | E | 1 | 17.739 | 63.087 | 63.589 | 0.68 | 61.03 |
| 3782 | C4 | MON | E | 1 | 18.513 | 61.940 | 63.159 | 0.68 | 61.27 |
| 3783 | C8 | MON | E | 1 | 18.041 | 62.421 | 65.826 | 0.68 | 61.34 |
| 3784 | C3 | MON | E | 1 | 18.743 | 61.167 | 65.343 | 0.68 | 54.14 |
| 3785 | C1 | MON | E | 1 | 18.987 | 60.960 | 64.029 | 0.68 | 52.79 |
| 3786 | C2 | MON | E | 1 | 19.619 | 59.651 | 63.617 | 0.68 | 64.04 |
| 3787 | O7 | MON | E | 1 | 20.348 | 59.164 | 64.675 | 0.68 | 55.74 |
| 3788 | C6 | MON | E | 1 | 18.393 | 58.583 | 63.328 | 0.68 | 53.08 |
| 3789 | F15 | MON | E | 1 | 19.132 | 57.425 | 63.011 | 0.68 | 57.55 |
| 3790 | F14 | MON | E | 1 | 17.755 | 58.998 | 62.281 | 0.68 | 54.49 |
| 3791 | F13 | MON | E | 1 | 17.552 | 58.341 | 64.329 | 0.68 | 53.53 |
| 3792 | C5 | MON | E | 1 | 20.593 | 59.844 | 62.509 | 0.68 | 66.00 |
| 3793 | F12 | MON | E | 1 | 21.145 | 58.702 | 62.164 | 0.68 | 71.56 |
| 3794 | F11 | MON | E | 1 | 20.052 | 60.351 | 61.466 | 0.68 | 54.24 |
| 3795 | F10 | MON | E | 1 | 21.505 | 60.695 | 62.917 | 0.68 | 58.61 |
| 3796 | S18 | MON | E | 1 | 15.447 | 64.822 | 65.753 | 0.68 | 83.56 |
| 3797 | O21 | MON | E | 1 | 14.518 | 65.274 | 64.634 | 0.68 | 93.03 |
| 3798 | O22 | MON | E | 1 | 15.130 | 65.547 | 66.980 | 0.68 | 80.99 |
| 3799 | C20 | MON | E | 1 | 14.228 | 63.373 | 65.915 | 0.68 | 77.88 |
| 3800 | C24 | MON | E | 1 | 13.224 | 63.230 | 66.928 | 0.68 | 68.92 |
| 3801 | C29 | MON | E | 1 | 12.436 | 62.100 | 67.028 | 0.68 | 73.62 |
| 3802 | C31 | MON | E | 1 | 12.646 | 60.970 | 66.232 | 0.68 | 70.40 |
| 3803 | C30 | MON | E | 1 | 13.636 | 61.041 | 65.250 | 0.68 | 82.63 |
| 3804 | C25 | MON | E | 1 | 14.431 | 62.235 | 65.055 | 0.68 | 79.09 |
| 3805 | O | HOH | W | 2 | 28.763 | 91.475 | 95.071 | 1.00 | 39.87 |
| 3806 | O | HOH | W | 3 | 46.618 | 53.254 | 66.806 | 1.00 | 48.01 |
| 3807 | O | HOH | W | 4 | 28.768 | 71.285 | 63.182 | 1.00 | 59.05 |
| 3808 | O | HOH | W | 5 | 28.489 | 58.092 | 96.686 | 1.00 | 20.96 |
| 3809 | O | HOH | W | 6 | 33.736 | 54.376 | 64.043 | 1.00 | 59.64 |
| 3810 | O | HOH | W | 7 | 14.597 | 69.385 | 75.168 | 1.00 | 72.38 |
| 3811 | O | HOH | W | 8 | 18.802 | 70.558 | 72.353 | 1.00 | 53.73 |
| 3812 | O | HOH | W | 9 | 33.884 | 83.187 | 70.518 | 1.00 | 55.24 |
| 3813 | O | HOH | W | 10 | 22.912 | 96.047 | 71.861 | 1.00 | 55.92 |
| 3814 | O | HOH | W | 11 | 18.761 | 96.307 | 66.664 | 1.00 | 60.45 |
| 3815 | O | HOH | W | 12 | 30.903 | 80.413 | 80.693 | 1.00 | 40.55 |
| 3816 | O | HOH | W | 13 | 25.337 | 91.192 | 77.632 | 1.00 | 72.99 |
| 3817 | O | HOH | W | 14 | 34.871 | 85.846 | 83.379 | 1.00 | 46.70 |
| 3818 | O | HOH | W | 15 | 29.736 | 65.196 | 80.159 | 1.00 | 57.86 |
| 3819 | O | HOH | W | 16 | 23.629 | 94.190 | 90.119 | 1.00 | 28.94 |
| 3820 | O | HOH | W | 17 | 17.330 | 58.608 | 82.548 | 1.00 | 54.28 |
| 3821 | O | HOH | W | 18 | 34.366 | 59.527 | 79.752 | 1.00 | 55.89 |
| 3822 | O | HOH | W | 19 | 44.065 | 83.193 | 100.457 | 1.00 | 79.63 |
| 3823 | O | HOH | W | 20 | 30.075 | 69.132 | 105.305 | 1.00 | 75.54 |
| 3824 | O | HOH | W | 21 | 30.061 | 70.867 | 103.341 | 1.00 | 62.79 |
| 3825 | O | HOH | W | 22 | 14.365 | 58.842 | 111.595 | 1.00 | 56.40 |
| 3826 | O | HOH | W | 23 | 2.852 | 72.225 | 103.788 | 1.00 | 58.58 |
| 3827 | O | HOH | W | 24 | 8.238 | 72.237 | 111.123 | 1.00 | 64.18 |
| 3828 | O | HOH | W | 25 | 35.890 | 83.185 | 103.291 | 1.00 | 37.51 |
| 3829 | O | HOH | W | 26 | 40.472 | 81.808 | 96.420 | 1.00 | 50.13 |
| 3830 | O | HOH | W | 27 | 34.642 | 91.721 | 104.562 | 1.00 | 51.95 |
| 3831 | O | HOH | W | 28 | 26.151 | 85.227 | 103.132 | 1.00 | 62.19 |
| 3832 | O | HOH | W | 29 | 20.708 | 86.222 | 94.639 | 1.00 | 37.83 |
| 3833 | O | HOH | W | 30 | 20.141 | 75.780 | 95.140 | 1.00 | 28.16 |
| 3834 | O | HOH | W | 31 | 23.371 | 64.729 | 103.731 | 1.00 | 54.24 |
| 3835 | O | HOH | W | 32 | 29.196 | 52.130 | 105.039 | 1.00 | 54.15 |
| 3836 | O | HOH | W | 33 | 21.723 | 58.410 | 113.952 | 1.00 | 56.23 |
| 3837 | O | HOH | W | 34 | 14.086 | 56.629 | 108.755 | 1.00 | 64.61 |
| 3838 | O | HOH | W | 35 | 14.464 | 63.003 | 85.862 | 1.00 | 60.12 |
| 3839 | O | HOH | W | 36 | 34.911 | 56.785 | 97.989 | 1.00 | 50.01 |
| 3840 | O | HOH | W | 37 | 38.951 | 63.054 | 106.082 | 1.00 | 52.37 |
| 3841 | O | HOH | W | 38 | 31.326 | 77.306 | 84.904 | 1.00 | 45.71 |
| 3842 | O | HOH | W | 39 | 29.270 | 92.583 | 85.702 | 1.00 | 35.13 |
| 3843 | O | HOH | W | 40 | 47.229 | 63.071 | 97.529 | 1.00 | 61.45 |
| 3844 | O | HOH | W | 41 | 46.228 | 60.875 | 98.976 | 1.00 | 63.36 |
| 3845 | O | HOH | W | 42 | 40.920 | 51.820 | 95.056 | 1.00 | 57.34 |
| 3846 | O | HOH | W | 43 | 47.377 | 55.626 | 89.451 | 1.00 | 49.82 |
| 3847 | O | HOH | W | 44 | 45.228 | 58.061 | 81.927 | 1.00 | 67.31 |
| 3848 | O | HOH | W | 45 | 37.354 | 61.041 | 81.512 | 1.00 | 55.21 |
| 3849 | O | HOH | W | 46 | 26.553 | 61.536 | 86.551 | 1.00 | 48.38 |
| 3850 | O | HOH | W | 47 | 23.871 | 57.832 | 86.725 | 1.00 | 63.15 |
| 3851 | O | HOH | W | 48 | 21.351 | 75.888 | 87.379 | 1.00 | 55.36 |
| 3852 | O | HOH | W | 49 | 16.336 | 66.979 | 82.247 | 1.00 | 52.35 |
| 3853 | O | HOH | W | 50 | 20.491 | 78.894 | 89.268 | 1.00 | 49.24 |
| 3854 | O | HOH | W | 51 | 8.641 | 79.155 | 97.921 | 1.00 | 41.86 |
| 3855 | O | HOH | W | 52 | 28.754 | 57.106 | 99.998 | 1.00 | 22.16 |
| 3856 | O | HOH | W | 53 | 27.743 | 79.059 | 92.733 | 1.00 | 24.38 |
| 3857 | O | HOH | W | 54 | 25.166 | 51.962 | 95.208 | 1.00 | 28.66 |
| 3858 | O | HOH | W | 55 | 23.573 | 60.455 | 101.796 | 1.00 | 26.48 |
| 3859 | O | HOH | W | 56 | 26.250 | 69.724 | 78.744 | 1.00 | 31.75 |
| 3860 | O | HOH | W | 57 | 34.323 | 60.294 | 100.383 | 1.00 | 40.53 |
| 3861 | O | HOH | W | 58 | 27.486 | 72.953 | 88.697 | 1.00 | 28.68 |
| 3862 | O | HOH | W | 59 | 34.889 | 90.573 | 88.517 | 1.00 | 31.09 |
| 3863 | O | HOH | W | 60 | 33.283 | 58.848 | 95.676 | 1.00 | 31.39 |
| 3864 | O | HOH | W | 61 | 30.953 | 58.858 | 97.106 | 1.00 | 23.93 |
| 3865 | O | HOH | W | 62 | 22.512 | 52.171 | 95.445 | 1.00 | 27.62 |
| 3866 | O | HOH | W | 63 | 20.598 | 70.234 | 76.468 | 1.00 | 51.57 |
| 3867 | O | HOH | W | 64 | 12.319 | 64.060 | 118.862 | 1.00 | 55.13 |
| 3868 | O | HOH | W | 65 | 31.209 | 90.825 | 94.490 | 1.00 | 39.05 |
| 3869 | O | HOH | W | 66 | 23.791 | 79.639 | 85.574 | 1.00 | 41.46 |
| 3870 | O | HOH | W | 67 | 37.984 | 84.467 | 81.167 | 1.00 | 33.22 |
| 3871 | O | HOH | W | 68 | 18.599 | 76.019 | 92.910 | 1.00 | 39.06 |
| 3872 | O | HOH | W | 69 | 28.327 | 53.170 | 101.909 | 1.00 | 35.64 |
| 3873 | O | HOH | W | 70 | 17.201 | 75.206 | 88.495 | 1.00 | 43.62 |
| 3874 | O | HOH | W | 71 | 19.557 | 52.534 | 71.813 | 1.00 | 43.84 |
| 3875 | O | HOH | W | 72 | 31.383 | 86.335 | 79.832 | 1.00 | 31.19 |
| 3876 | O | HOH | W | 73 | 45.534 | 69.874 | 81.890 | 1.00 | 70.44 |
| 3877 | O | HOH | W | 74 | 23.398 | 78.113 | 87.971 | 1.00 | 41.72 |
| 3878 | O | HOH | W | 75 | 24.985 | 79.636 | 81.937 | 1.00 | 40.70 |
| 3879 | O | HOH | W | 76 | 35.214 | 56.538 | 86.573 | 1.00 | 61.87 |
| 3880 | O | HOH | W | 77 | 8.312 | 72.048 | 61.316 | 1.00 | 51.15 |
| 3881 | O | HOH | W | 78 | 54.583 | 67.580 | 79.761 | 1.00 | 92.40 |
| 3882 | O | HOH | W | 79 | 30.058 | 58.304 | 87.420 | 1.00 | 45.56 |
| 3883 | O | HOH | W | 80 | 15.658 | 57.937 | 72.486 | 1.00 | 49.88 |
| 3884 | O | HOH | W | 81 | 38.406 | 60.747 | 103.355 | 1.00 | 49.62 |
| 3885 | O | HOH | W | 82 | 45.112 | 73.835 | 79.366 | 1.00 | 57.23 |
| 3886 | O | HOH | W | 83 | 24.311 | 56.019 | 87.791 | 1.00 | 49.24 |
| 3887 | O | HOH | W | 84 | 11.054 | 91.063 | 75.823 | 1.00 | 62.21 |
| 3888 | O | HOH | W | 85 | 25.110 | 79.361 | 60.187 | 1.00 | 70.53 |
| 3889 | O | HOH | W | 86 | 21.814 | 61.622 | 88.069 | 1.00 | 45.93 |
| 3890 | O | HOH | W | 87 | 17.326 | 70.560 | 74.981 | 1.00 | 49.19 |
| 3891 | O | HOH | W | 88 | 27.869 | 64.366 | 81.736 | 1.00 | 72.04 |
| 3892 | O | HOH | W | 89 | 32.821 | 76.136 | 112.204 | 1.00 | 54.40 |
| 3893 | O | HOH | W | 90 | 33.796 | 83.623 | 82.106 | 1.00 | 54.68 |
| 3894 | O | HOH | W | 91 | 18.105 | 70.460 | 58.689 | 1.00 | 51.59 |
| 3895 | O | HOH | W | 92 | 21.706 | 82.125 | 101.976 | 1.00 | 57.32 |
| 3896 | O | HOH | W | 93 | 27.381 | 71.426 | 59.765 | 1.00 | 58.02 |
| 3897 | O | HOH | W | 94 | 27.472 | 55.467 | 86.841 | 1.00 | 49.46 |
| 3898 | O | HOH | W | 95 | 22.395 | 88.520 | 95.592 | 1.00 | 50.28 |
| 3899 | O | HOH | W | 96 | −5.048 | 60.064 | 109.413 | 1.00 | 67.72 |
| 3900 | O | HOH | W | 97 | 14.636 | 51.620 | 97.555 | 1.00 | 72.51 |
| 3901 | O | HOH | W | 98 | 46.663 | 68.262 | 64.814 | 1.00 | 59.94 |
| 3902 | O | HOH | W | 99 | 51.526 | 66.481 | 63.513 | 1.00 | 60.60 |
| 3903 | O | HOH | W | 100 | 12.853 | 73.278 | 56.067 | 1.00 | 70.36 |
| 3904 | O | HOH | W | 101 | 17.395 | 50.850 | 69.827 | 1.00 | 51.25 |
| 3905 | O | HOH | W | 102 | 37.188 | 84.773 | 68.441 | 1.00 | 53.49 |
| 3906 | O | HOH | W | 103 | 26.301 | 84.156 | 54.117 | 1.00 | 74.97 |
| 3907 | O | HOH | W | 104 | 15.569 | 53.268 | 106.007 | 1.00 | 68.93 |
| 3908 | O | HOH | W | 105 | 11.106 | 82.832 | 80.170 | 1.00 | 52.24 |
| 3909 | O | HOH | W | 106 | 9.827 | 84.209 | 83.460 | 1.00 | 64.61 |
| 3910 | O | HOH | W | 107 | 39.542 | 90.057 | 76.485 | 1.00 | 58.59 |
| 3911 | O | HOH | W | 108 | 42.229 | 77.453 | 63.523 | 1.00 | 76.86 |
| 3912 | O | HOH | W | 109 | 27.370 | 67.242 | 78.876 | 1.00 | 41.58 |
| 3913 | O | HOH | W | 110 | 37.384 | 55.312 | 98.950 | 1.00 | 56.58 |
| 3914 | O | HOH | W | 111 | 31.815 | 58.721 | 100.040 | 1.00 | 43.21 |
| 3915 | O | HOH | W | 112 | 31.506 | 55.285 | 100.972 | 1.00 | 63.15 |
| 3916 | O | HOH | W | 113 | 38.178 | 54.915 | 91.789 | 1.00 | 58.54 |
| 3917 | O | HOH | W | 114 | 41.103 | 63.301 | 102.308 | 1.00 | 52.30 |
| 3918 | O | HOH | W | 115 | 19.518 | 79.361 | 85.632 | 1.00 | 56.13 |
| 3919 | O | HOH | W | 116 | 17.896 | 87.232 | 86.207 | 1.00 | 53.56 |
| 3920 | O | HOH | W | 117 | 13.629 | 61.460 | 82.107 | 1.00 | 53.88 |
| 3921 | O | HOH | W | 118 | 16.966 | 63.736 | 84.587 | 1.00 | 65.21 |
| 3922 | O | HOH | W | 119 | 28.949 | 48.535 | 78.225 | 1.00 | 54.95 |
| 3923 | O | HOH | W | 120 | 31.452 | 49.739 | 78.799 | 1.00 | 55.62 |
| 3924 | O | HOH | W | 121 | 19.965 | 71.727 | 82.620 | 1.00 | 60.17 |
| 3925 | O | HOH | W | 122 | 0.228 | 75.953 | 71.014 | 1.00 | 76.55 |
| 3926 | O | HOH | W | 123 | 0.158 | 73.725 | 64.810 | 1.00 | 65.34 |
| 3927 | O | HOH | W | 124 | 43.121 | 67.781 | 107.834 | 1.00 | 68.84 |
| 3928 | O | HOH | W | 125 | 42.068 | 71.355 | 112.215 | 1.00 | 71.87 |
| 3929 | O | HOH | W | 126 | 40.486 | 64.768 | 112.276 | 1.00 | 58.12 |
| 3930 | O | HOH | W | 127 | 35.045 | 83.128 | 108.243 | 1.00 | 81.25 |
| 3931 | O | HOH | W | 128 | 33.826 | 80.790 | 112.590 | 1.00 | 57.74 |
| 3932 | O | HOH | W | 129 | 30.755 | 80.445 | 110.066 | 1.00 | 68.05 |
| 3933 | O | HOH | W | 130 | 23.881 | 66.592 | 107.341 | 1.00 | 55.54 |
| 3934 | O | HOH | W | 131 | 21.181 | 65.654 | 120.113 | 1.00 | 66.27 |
| 3935 | O | HOH | W | 132 | −1.340 | 56.710 | 98.372 | 1.00 | 70.45 |
| 3936 | O | HOH | W | 133 | −3.291 | 66.354 | 109.085 | 1.00 | 76.45 |
| 3937 | O | HOH | W | 134 | −0.721 | 72.343 | 105.882 | 1.00 | 90.08 |
| 3938 | O | HOH | W | 135 | 18.563 | 85.475 | 104.514 | 1.00 | 66.59 |
| 3939 | O | HOH | W | 136 | 21.187 | 86.326 | 106.734 | 1.00 | 62.94 |
| 3940 | O | HOH | W | 137 | 24.589 | 87.631 | 107.644 | 1.00 | 51.47 |
| 3941 | O | HOH | W | 138 | 25.316 | 87.133 | 104.100 | 1.00 | 69.03 |
| 3942 | O | HOH | W | 139 | 19.895 | 84.386 | 100.975 | 1.00 | 66.18 |
| 3943 | O | HOH | W | 140 | 24.239 | 84.741 | 106.617 | 1.00 | 59.66 |
| 3944 | O | HOH | W | 141 | 41.754 | 83.998 | 98.620 | 1.00 | 71.11 |
| 3945 | O | HOH | W | 142 | 32.225 | 59.750 | 103.596 | 1.00 | 56.52 |
| 3946 | O | HOH | W | 143 | 11.765 | 49.929 | 100.176 | 1.00 | 64.61 |
| 3947 | O | HOH | W | 144 | 7.717 | 53.143 | 96.964 | 1.00 | 63.33 |
| 3948 | O | HOH | W | 145 | 40.860 | 54.461 | 84.748 | 1.00 | 90.92 |
| 3949 | O | HOH | W | 146 | 15.363 | 84.742 | 107.809 | 1.00 | 64.26 |
| 3950 | O | HOH | W | 147 | 15.633 | 85.708 | 99.119 | 1.00 | 67.76 |
| 3951 | O | HOH | W | 148 | 7.601 | 76.855 | 91.970 | 1.00 | 60.58 |
| 3952 | O | HOH | W | 149 | 9.623 | 82.594 | 90.860 | 1.00 | 72.36 |
| 3953 | O | HOH | W | 150 | 24.202 | 89.335 | 92.759 | 1.00 | 43.28 |
| 3954 | O | HOH | W | 151 | 24.526 | 64.149 | 74.154 | 1.00 | 36.12 |
| 3955 | O | HOH | W | 152 | 45.473 | 55.901 | 61.786 | 1.00 | 65.50 |
| 3956 | O | HOH | W | 153 | 29.439 | 74.941 | 88.426 | 1.00 | 26.89 |
| 3957 | O | HOH | W | 154 | 37.914 | 91.765 | 66.016 | 1.00 | 90.43 |
| 3958 | O | HOH | W | 155 | 14.460 | 76.420 | 92.234 | 1.00 | 55.50 |
| 3959 | O | HOH | W | 156 | 8.236 | 67.697 | 61.549 | 1.00 | 63.32 |
| 3960 | O | HOH | W | 157 | 29.107 | 60.295 | 79.228 | 1.00 | 43.90 |
| 3961 | O | HOH | W | 158 | 26.638 | 76.154 | 60.795 | 1.00 | 58.93 |
| 3962 | O | HOH | W | 159 | 33.709 | 80.210 | 87.528 | 1.00 | 38.57 |
| 3963 | O | HOH | W | 160 | 40.715 | 82.585 | 84.782 | 1.00 | 68.42 |
| 3964 | O | HOH | W | 161 | 11.233 | 56.537 | 68.262 | 1.00 | 63.15 |
| 3965 | O | HOH | W | 162 | 21.975 | 64.415 | 85.175 | 1.00 | 40.81 |
| 3966 | O | HOH | W | 163 | 21.602 | 75.374 | 90.625 | 1.00 | 41.66 |
| 3967 | O | HOH | W | 164 | 43.202 | 83.556 | 90.439 | 1.00 | 44.45 |
| 3968 | O | HOH | W | 165 | 47.305 | 61.806 | 77.748 | 1.00 | 56.29 |
| 3969 | O | HOH | W | 166 | 16.383 | 73.291 | 75.523 | 1.00 | 44.82 |
| 3970 | O | HOH | W | 167 | 29.502 | 82.147 | 80.221 | 1.00 | 45.92 |
| 3971 | O | HOH | W | 168 | 55.133 | 60.635 | 74.223 | 1.00 | 74.10 |
| 3972 | O | HOH | W | 169 | 53.642 | 66.189 | 77.639 | 1.00 | 67.27 |
| 3973 | O | HOH | W | 170 | 32.023 | 76.158 | 86.775 | 1.00 | 50.78 |
| 3974 | O | HOH | W | 171 | 42.714 | 59.651 | 72.633 | 1.00 | 45.98 |
| 3975 | O | HOH | W | 172 | 26.008 | 50.088 | 106.857 | 1.00 | 40.13 |
| 3976 | O | HOH | W | 173 | 24.123 | 74.327 | 110.723 | 1.00 | 62.76 |
| 3977 | O | HOH | W | 174 | 43.725 | 70.645 | 61.138 | 1.00 | 66.39 |
| 3978 | O | HOH | W | 175 | 14.448 | 62.212 | 116.064 | 1.00 | 62.04 |
| 3979 | O | HOH | W | 176 | 22.321 | 95.254 | 69.714 | 1.00 | 63.47 |
| 3980 | O | HOH | W | 177 | 42.597 | 84.404 | 72.736 | 1.00 | 66.33 |
| 3981 | O | HOH | W | 178 | 39.815 | 56.522 | 94.119 | 1.00 | 51.02 |
| 3982 | O | HOH | W | 179 | 48.572 | 80.734 | 89.655 | 1.00 | 58.93 |
| 3983 | O | HOH | W | 180 | 17.082 | 49.172 | 97.561 | 1.00 | 55.92 |
| 3984 | O | HOH | W | 181 | 33.421 | 65.376 | 55.404 | 1.00 | 53.64 |
| 3985 | O | HOH | W | 182 | 30.691 | 93.733 | 74.873 | 1.00 | 60.59 |
| 3986 | O | HOH | W | 183 | 43.334 | 61.083 | 90.066 | 1.00 | 52.70 |
| 3987 | O | HOH | W | 184 | 47.929 | 50.409 | 83.769 | 1.00 | 73.41 |
| 3988 | O | HOH | W | 185 | 27.889 | 62.068 | 57.384 | 1.00 | 58.23 |
| 3989 | O | HOH | W | 186 | 2.632 | 58.059 | 112.990 | 1.00 | 55.10 |
| 3990 | O | HOH | W | 187 | 49.666 | 81.692 | 68.959 | 1.00 | 69.04 |
| 3991 | O | HOH | W | 188 | 21.840 | 96.023 | 88.622 | 1.00 | 56.16 |
| 3992 | O | HOH | W | 189 | 20.531 | 81.031 | 91.634 | 1.00 | 48.68 |
| 3993 | O | HOH | W | 190 | 13.144 | 52.725 | 91.050 | 1.00 | 74.72 |
| 3994 | O | HOH | W | 191 | 16.078 | 55.158 | 84.219 | 1.00 | 76.76 |
| 3995 | O | HOH | W | 192 | 21.973 | 94.500 | 78.308 | 1.00 | 54.35 |
| 3996 | O | HOH | W | 193 | 16.039 | 69.725 | 82.713 | 1.00 | 58.13 |
| 3997 | O | HOH | W | 194 | 29.925 | 92.916 | 101.327 | 1.00 | 55.39 |
| 3998 | O | HOH | W | 195 | 39.875 | 83.519 | 69.873 | 1.00 | 76.51 |
| 3999 | O | HOH | W | 196 | 33.547 | 79.050 | 116.541 | 1.00 | 58.96 |
| 4000 | O | HOH | W | 197 | 31.777 | 97.620 | 69.127 | 1.00 | 58.13 |
| 4001 | O | HOH | W | 198 | 19.289 | 81.660 | 108.573 | 1.00 | 54.02 |
| 4002 | O | HOH | W | 199 | 19.307 | 90.323 | 83.858 | 1.00 | 73.87 |
| 4003 | O | HOH | W | 200 | 42.438 | 81.165 | 77.189 | 1.00 | 57.30 |
| 4004 | O | HOH | W | 201 | 24.719 | 88.867 | 111.158 | 1.00 | 71.06 |
| 4005 | O | HOH | W | 202 | 14.752 | 82.372 | 79.831 | 1.00 | 50.46 |
| 4006 | O | HOH | W | 203 | 29.593 | 62.478 | 107.228 | 1.00 | 56.35 |
| 4007 | O | HOH | W | 204 | 44.276 | 54.216 | 84.906 | 1.00 | 63.06 |
| 4008 | O | HOH | W | 205 | 43.714 | 79.770 | 68.714 | 1.00 | 53.81 |
| 4009 | O | HOH | W | 206 | 10.195 | 74.128 | 58.090 | 1.00 | 67.94 |
| 4010 | O | HOH | W | 207 | 52.198 | 56.890 | 69.158 | 1.00 | 82.99 |
| 4011 | O | HOH | W | 208 | 41.501 | 55.621 | 88.559 | 1.00 | 58.64 |
| 4012 | O | HOH | W | 209 | 12.404 | 54.801 | 113.084 | 1.00 | 61.38 |
| 4013 | O | HOH | W | 210 | 20.935 | 85.398 | 102.822 | 1.00 | 59.61 |
| 4014 | O | HOH | W | 211 | 1.884 | 58.378 | 100.966 | 1.00 | 52.81 |
| 4015 | O | HOH | W | 212 | 44.292 | 66.241 | 113.852 | 1.00 | 62.95 |
| 4016 | O | HOH | W | 213 | 20.461 | 99.227 | 68.950 | 1.00 | 81.54 |
| 4017 | O | HOH | W | 214 | 54.282 | 63.551 | 95.771 | 1.00 | 73.65 |
| 4018 | O | HOH | W | 215 | 52.630 | 59.645 | 95.163 | 1.00 | 61.37 |
| 4019 | O | HOH | W | 216 | 43.847 | 74.747 | 108.470 | 1.00 | 47.79 |
| 4020 | O | HOH | W | 217 | 13.821 | 49.683 | 70.834 | 1.00 | 62.38 |
| 4021 | O | HOH | W | 218 | 30.134 | 58.491 | 54.366 | 1.00 | 60.17 |
| 4022 | O | HOH | W | 219 | 19.590 | 62.628 | 116.493 | 1.00 | 64.94 |
| 4023 | O | HOH | W | 220 | 10.033 | 74.052 | 109.418 | 1.00 | 60.61 |
| 4024 | O | HOH | W | 221 | 37.721 | 52.622 | 84.421 | 1.00 | 52.75 |
| 4025 | O | HOH | W | 222 | 4.965 | 79.234 | 106.393 | 1.00 | 54.50 |
| 4026 | O | HOH | W | 223 | 35.059 | 78.092 | 85.271 | 1.00 | 79.95 |
| 4027 | O | HOH | W | 224 | 41.104 | 59.402 | 59.846 | 1.00 | 67.70 |
| 4028 | O | HOH | W | 225 | 0.204 | 75.441 | 102.085 | 1.00 | 72.03 |
| 4029 | O | HOH | W | 226 | 27.249 | 75.955 | 121.137 | 1.00 | 96.12 |
| 4030 | O | HOH | W | 227 | 40.480 | 81.950 | 80.258 | 1.00 | 69.28 |
| 4031 | O | HOH | W | 228 | 13.904 | 89.137 | 105.643 | 1.00 | 73.36 |
| 4032 | O | HOH | W | 229 | 18.966 | 46.160 | 73.696 | 1.00 | 60.83 |
| 4033 | O | HOH | W | 230 | 25.204 | 95.032 | 76.561 | 1.00 | 55.01 |
| 4034 | O | HOH | W | 231 | 44.154 | 87.852 | 102.404 | 1.00 | 81.52 |
| 4035 | O | HOH | W | 232 | 15.734 | 50.286 | 102.580 | 1.00 | 73.57 |
| 4036 | O | HOH | W | 233 | 34.230 | 91.950 | 53.379 | 1.00 | 64.79 |
| 4037 | O | HOH | W | 234 | 34.028 | 88.196 | 54.572 | 1.00 | 76.62 |
| 4038 | O | HOH | W | 235 | 6.510 | 81.703 | 81.936 | 1.00 | 67.44 |
| 4039 | O | HOH | W | 236 | 31.250 | 46.769 | 80.109 | 1.00 | 59.73 |
| 4040 | O | HOH | W | 237 | −4.328 | 66.322 | 104.598 | 1.00 | 72.03 |
| 4041 | O | HOH | W | 238 | −5.175 | 65.411 | 111.235 | 1.00 | 80.31 |
| 4042 | O | HOH | W | 239 | 42.619 | 86.327 | 93.196 | 1.00 | 55.26 |
| 4043 | O | HOH | W | 240 | 53.056 | 58.639 | 77.745 | 1.00 | 67.77 |
| 4044 | O | HOH | W | 241 | 55.986 | 68.627 | 74.749 | 1.00 | 67.32 |
| 4045 | O | HOH | W | 242 | 26.578 | 51.838 | 109.039 | 1.00 | 68.47 |
| 4046 | O | HOH | W | 243 | 27.401 | 98.889 | 69.713 | 1.00 | 65.37 |
| 4047 | O | HOH | W | 244 | 38.092 | 57.444 | 100.192 | 1.00 | 67.48 |
| 4048 | O | HOH | W | 245 | 5.883 | 60.647 | 120.458 | 1.00 | 62.41 |
| 4049 | O | HOH | W | 246 | 24.874 | 95.610 | 80.166 | 1.00 | 65.09 |
| 4050 | O | HOH | W | 247 | 31.317 | 98.945 | 72.700 | 1.00 | 55.79 |
| 4051 | O | HOH | W | 248 | 42.019 | 81.867 | 65.318 | 1.00 | 64.05 |
| 4052 | O | HOH | W | 249 | 11.354 | 77.091 | 54.840 | 1.00 | 58.78 |
| 4053 | O | HOH | W | 250 | 2.257 | 54.062 | 96.344 | 1.00 | 69.03 |
| 4054 | O | HOH | W | 251 | 21.865 | 103.114 | 67.026 | 1.00 | 88.24 |
| 4055 | O | HOH | W | 252 | 25.783 | 79.878 | 112.404 | 1.00 | 71.73 |
| 4056 | O | HOH | W | 254 | 15.451 | 47.612 | 78.503 | 1.00 | 58.78 |
| 4057 | O | HOH | W | 255 | 20.013 | 42.200 | 74.057 | 1.00 | 54.88 |
| 4058 | O | HOH | W | 256 | 47.186 | 86.427 | 95.913 | 1.00 | 69.52 |
| 4059 | O | HOH | W | 257 | 42.710 | 86.262 | 96.986 | 1.00 | 69.38 |
| 4060 | O | HOH | W | 258 | 32.597 | 86.812 | 48.714 | 1.00 | 80.06 |
| 4061 | O | HOH | W | 259 | 0.686 | 79.870 | 83.804 | 1.00 | 69.66 |
| 4062 | O | HOH | W | 260 | 53.472 | 56.751 | 82.708 | 1.00 | 63.88 |
| 4063 | O | HOH | W | 261 | 8.076 | 56.013 | 119.805 | 1.00 | 75.94 |
| 4064 | O | HOH | W | 262 | 3.711 | 56.666 | 118.238 | 1.00 | 61.92 |
| 4065 | O | HOH | W | 263 | 8.577 | 61.095 | 122.359 | 1.00 | 79.31 |
| 4066 | O | HOH | W | 264 | 1.591 | 76.706 | 58.691 | 1.00 | 61.27 |
| 4067 | O | HOH | W | 265 | 9.902 | 70.303 | 56.931 | 1.00 | 64.53 |
| 4068 | O | HOH | W | 266 | 3.286 | 73.378 | 61.337 | 1.00 | 70.97 |
| 4069 | O | HOH | W | 267 | 1.544 | 57.048 | 89.873 | 1.00 | 66.17 |
| 4070 | O | HOH | W | 268 | 58.203 | 52.547 | 96.098 | 1.00 | 59.53 |
| 4071 | O | HOH | W | 269 | 55.863 | 59.303 | 86.462 | 1.00 | 86.54 |
| 4072 | O | HOH | W | 270 | 39.454 | 93.055 | 102.022 | 1.00 | 73.13 |
| 4073 | O | HOH | W | 271 | 57.151 | 62.618 | 85.040 | 1.00 | 60.06 |
| 4074 | O | HOH | W | 272 | 61.388 | 66.554 | 79.136 | 1.00 | 75.24 |
| 4075 | O | HOH | W | 273 | 16.444 | 65.572 | 76.282 | 1.00 | 48.26 |
| 4076 | O | HOH | W | 274 | −7.019 | 56.050 | 104.617 | 1.00 | 68.20 |
| 4077 | O | HOH | W | 275 | 41.260 | 91.689 | 99.772 | 1.00 | 75.60 |
| 4078 | O | HOH | W | 276 | 7.750 | 72.640 | 69.911 | 1.00 | 65.48 |
| 4079 | O | HOH | W | 277 | −0.534 | 56.593 | 93.766 | 1.00 | 55.74 |
| 4080 | O | HOH | W | 278 | 45.690 | 60.610 | 73.854 | 1.00 | 55.45 |
| 4081 | O | HOH | W | 279 | 43.232 | 79.073 | 98.278 | 1.00 | 68.39 |
| 4082 | O | HOH | W | 280 | 33.018 | 56.513 | 99.871 | 1.00 | 47.87 |
| 4083 | O | HOH | W | 281 | 12.875 | 91.417 | 62.202 | 1.00 | 50.60 |
| 4084 | O | HOH | W | 282 | 23.740 | 91.795 | 87.843 | 1.00 | 50.12 |
| 4085 | O | HOH | W | 283 | 33.901 | 87.154 | 60.631 | 1.00 | 57.12 |
| 4086 | O | HOH | W | 284 | 50.111 | 69.534 | 88.258 | 1.00 | 61.69 |
| 4087 | O | HOH | W | 285 | 22.172 | 79.896 | 108.037 | 1.00 | 49.77 |
| 4088 | O | HOH | W | 286 | 21.199 | 90.174 | 96.915 | 1.00 | 62.16 |
| 4089 | O | HOH | W | 287 | 41.370 | 77.951 | 75.054 | 1.00 | 93.44 |
| 4090 | O | HOH | W | 288 | −3.670 | 64.205 | 109.234 | 1.00 | 80.74 |
| 4091 | O | HOH | W | 289 | 44.099 | 65.205 | 84.325 | 1.00 | 65.13 |
| 4092 | O | HOH | W | 290 | 18.230 | 84.669 | 98.089 | 1.00 | 73.24 |
| 4093 | O | HOH | W | 291 | 6.251 | 58.894 | 82.343 | 1.00 | 56.09 |
| 4094 | O | HOH | W | 292 | 4.533 | 87.103 | 81.626 | 1.00 | 62.59 |
| 4095 | O | HOH | W | 293 | 39.869 | 75.490 | 108.817 | 1.00 | 45.54 |
| 4096 | O | HOH | W | 294 | 23.699 | 54.140 | 107.333 | 1.00 | 46.52 |
| 4097 | O | HOH | W | 295 | 44.615 | 81.058 | 87.775 | 1.00 | 51.98 |
| 4098 | O | HOH | W | 296 | 5.134 | 86.562 | 85.564 | 1.00 | 59.40 |
| 4099 | O | HOH | W | 297 | 6.400 | 75.372 | 58.580 | 1.00 | 95.89 |
| 4100 | O | HOH | W | 298 | 15.511 | 101.122 | 67.844 | 1.00 | 83.89 |
| 4101 | O | HOH | W | 299 | 15.805 | 52.427 | 83.025 | 1.00 | 54.36 |
| 4102 | O | HOH | W | 300 | 31.021 | 74.527 | 57.113 | 1.00 | 58.88 |
| 4103 | O | HOH | W | 301 | 27.315 | 76.686 | 57.795 | 1.00 | 64.74 |
| 4104 | O | HOH | X | 302 | 16.794 | 73.496 | 92.416 | 1.00 | 43.24 |
| 4105 | O | HOH | X | 303 | 25.310 | 69.852 | 104.407 | 1.00 | 40.75 |
| 4106 | O | HOH | X | 304 | 14.601 | 56.696 | 93.871 | 1.00 | 64.02 |
| 4107 | O | HOH | X | 305 | 21.573 | 63.907 | 101.673 | 1.00 | 61.34 |
| 4108 | O | HOH | X | 306 | 47.225 | 61.635 | 66.073 | 1.00 | 72.40 |
| 4109 | O | HOH | X | 307 | 27.850 | 51.658 | 107.180 | 1.00 | 61.47 |
| 4110 | O | HOH | X | 308 | 4.828 | 61.226 | 96.660 | 1.00 | 63.69 |
| 4111 | O | HOH | X | 309 | 18.843 | 89.280 | 66.555 | 1.00 | 67.83 |
| 4112 | O | HOH | X | 310 | 31.275 | 70.993 | 63.564 | 1.00 | 76.09 |
| 4113 | O | HOH | X | 311 | 11.055 | 79.338 | 71.713 | 1.00 | 87.73 |
| 4114 | O | HOH | X | 312 | 35.732 | 92.142 | 66.782 | 1.00 | 70.59 |
| 4115 | O | HOH | X | 313 | 45.991 | 70.742 | 79.189 | 1.00 | 50.63 |
| 4116 | O | HOH | X | 314 | 42.103 | 62.040 | 87.599 | 1.00 | 58.63 |
| 4117 | O | HOH | X | 315 | 21.915 | 81.889 | 51.317 | 1.00 | 70.16 |
| 4118 | O | HOH | X | 316 | 19.123 | 72.199 | 60.303 | 1.00 | 49.30 |
| 4119 | O | HOH | X | 317 | 17.023 | 63.306 | 68.075 | 1.00 | 69.61 |
| 4120 | O | HOH | X | 318 | 20.375 | 72.024 | 116.048 | 1.00 | 68.20 |
| 4121 | O | HOH | X | 319 | 26.175 | 77.621 | 62.544 | 1.00 | 79.76 |
| 4122 | O | HOH | X | 320 | 44.798 | 57.627 | 62.912 | 1.00 | 96.29 |
| 4123 | O | HOH | X | 321 | 34.264 | 87.734 | 78.140 | 1.00 | 59.27 |
| 4124 | O | HOH | X | 322 | 36.913 | 85.471 | 83.195 | 1.00 | 68.12 |
| 4125 | O | HOH | X | 323 | 21.300 | 65.553 | 105.006 | 1.00 | 67.05 |
| 4126 | O | HOH | X | 324 | 22.477 | 83.111 | 92.028 | 1.00 | 45.27 |
| 4127 | O | HOH | X | 325 | 29.919 | 63.783 | 57.812 | 1.00 | 78.09 |
| 4128 | O | HOH | X | 326 | 21.773 | 58.872 | 81.134 | 1.00 | 50.84 |
| 4129 | O | HOH | X | 327 | 9.524 | 55.783 | 94.636 | 1.00 | 70.31 |
| 4130 | O | HOH | X | 328 | 49.945 | 71.633 | 88.156 | 1.00 | 60.20 |
| 4131 | O | HOH | X | 329 | 46.051 | 80.340 | 75.306 | 1.00 | 66.68 |
| 4132 | O | HOH | X | 330 | 19.780 | 53.571 | 86.923 | 1.00 | 52.32 |
| 4133 | O | HOH | X | 331 | 33.754 | 59.963 | 85.790 | 1.00 | 79.38 |
| 4134 | O | HOH | X | 332 | 44.741 | 56.622 | 88.452 | 1.00 | 83.35 |
| 4135 | O | HOH | X | 333 | 13.601 | 64.513 | 112.251 | 1.00 | 85.96 |
| 4136 | O | HOH | X | 334 | 24.193 | 61.120 | 87.313 | 1.00 | 54.44 |
| 4137 | O | HOH | X | 335 | 48.193 | 76.404 | 103.259 | 1.00 | 68.76 |
| 4138 | O | HOH | X | 336 | 9.407 | 73.167 | 71.266 | 1.00 | 74.67 |
| 4139 | O | HOH | X | 337 | 22.838 | 91.635 | 90.182 | 1.00 | 57.23 |

TABLE 4
ATOMIC COORDINATE DATA FOR HUMAN RARγ EMPLOYED IN THE
MOLECULAR REPLACEMENT SOLUTION OF HUMAN LXR LIGAND
BINDING DOMAIN CRYSTALS

| | ATOM | | | | | | | | |
|---|---|---|---|---|---|---|---|---|---|
| ATOM | TYPE | RESIDUE | PROTEIN # | # | X | Y | Z | BCC | O |
| 1 | CB | LEU | A | 182 | −27.418 | −10.420 | −1.042 | 1.00 | 32.61 |
| 2 | CG | LEU | A | 182 | −27.812 | −11.853 | −0.661 | 1.00 | 33.33 |
| 3 | CD1 | LEU | A | 182 | −26.844 | −12.934 | −1.224 | 1.00 | 33.15 |
| 4 | CD2 | LEU | A | 182 | −27.818 | −11.894 | 0.872 | 1.00 | 32.41 |
| 5 | C | LEU | A | 182 | −26.797 | −8.358 | −2.226 | 1.00 | 31.66 |
| 6 | O | LEU | A | 182 | −25.593 | −8.099 | −2.172 | 1.00 | 31.81 |
| 7 | N | LEU | A | 182 | −26.227 | −10.497 | −3.276 | 1.00 | 33.77 |
| 8 | CA | LEU | A | 182 | −27.267 | −9.788 | −2.446 | 1.00 | 33.01 |
| 9 | N | SER | A | 183 | −27.741 | −7.437 | −2.073 | 1.00 | 30.24 |
| 10 | CA | SER | A | 183 | −27.379 | −6.058 | −1.802 | 1.00 | 26.79 |
| 11 | CB | SER | A | 183 | −28.588 | −5.139 | −1.923 | 1.00 | 26.27 |
| 12 | OG | SER | A | 183 | −29.258 | −5.339 | −3.148 | 1.00 | 27.42 |
| 13 | C | SER | A | 183 | −26.871 | −6.058 | −0.370 | 1.00 | 25.85 |
| 14 | O | SER | A | 183 | −25.798 | −5.515 | −0.086 | 1.00 | 25.47 |
| 15 | N | PRO | A | 184 | −27.599 | −6.747 | 0.544 | 1.00 | 24.99 |
| 16 | CD | PRO | A | 184 | −28.906 | −7.426 | 0.422 | 1.00 | 22.22 |
| 17 | CA | PRO | A | 184 | −27.136 | −6.766 | 1.933 | 1.00 | 22.12 |
| 18 | CB | PRO | A | 184 | −28.236 | −7.546 | 2.655 | 1.00 | 23.65 |
| 19 | CG | PRO | A | 184 | −28.899 | −8.334 | 1.575 | 1.00 | 23.82 |
| 20 | C | PRO | A | 184 | −25.778 | −7.392 | 2.070 | 1.00 | 20.98 |
| 21 | O | PRO | A | 184 | −24.944 | −6.888 | 2.815 | 1.00 | 22.89 |
| 22 | N | ALA | A | 185 | −25.528 | −8.436 | 1.290 | 1.00 | 18.94 |
| 23 | CA | ALA | A | 185 | −24.245 | −9.136 | 1.293 | 1.00 | 17.11 |
| 24 | CB | ALA | A | 185 | −24.290 | −10.281 | 0.316 | 1.00 | 19.74 |
| 25 | C | ALA | A | 185 | −23.123 | −8.181 | 0.909 | 1.00 | 16.78 |
| 26 | O | ALA | A | 185 | −22.072 | −8.152 | 1.553 | 1.00 | 15.58 |
| 27 | N | LEU | A | 186 | −23.346 | −7.439 | −0.171 | 1.00 | 16.27 |
| 28 | CA | LEU | A | 186 | −22.402 | −6.451 | −0.636 | 1.00 | 17.77 |
| 29 | CB | LEU | A | 186 | −22.876 | −5.795 | −1.931 | 1.00 | 21.12 |
| 30 | CG | LEU | A | 186 | −22.329 | −6.276 | −3.281 | 1.00 | 25.48 |
| 31 | CD1 | LEU | A | 186 | −22.491 | −5.150 | −4.303 | 1.00 | 26.32 |
| 32 | CD2 | LEU | A | 186 | −20.847 | −6.665 | −3.196 | 1.00 | 26.08 |
| 33 | C | LEU | A | 186 | −22.222 | −5.354 | 0.407 | 1.00 | 16.80 |
| 34 | O | LEU | A | 186 | −21.121 | −4.846 | 0.587 | 1.00 | 17.47 |
| 35 | N | GLU | A | 187 | −23.287 | −4.971 | 1.090 | 1.00 | 14.83 |
| 36 | CA | GLU | A | 187 | −23.149 | −3.925 | 2.078 | 1.00 | 14.29 |
| 37 | CB | GLU | A | 187 | −24.498 | −3.501 | 2.644 | 1.00 | 13.39 |
| 38 | CG | GLU | A | 187 | −25.438 | −2.902 | 1.603 | 1.00 | 17.47 |
| 39 | CD | GLU | A | 187 | −24.911 | −1.645 | 0.907 | 1.00 | 18.66 |
| 40 | OE1 | GLU | A | 187 | −23.993 | −0.982 | 1.433 | 1.00 | 19.34 |
| 41 | OE2 | GLU | A | 187 | −25.446 | −1.303 | −0.166 | 1.00 | 18.74 |
| 42 | C | GLU | A | 187 | −22.255 | −4.371 | 3.196 | 1.00 | 15.23 |
| 43 | O | GLU | A | 187 | −21.495 | −3.579 | 3.717 | 1.00 | 14.48 |
| 44 | N | GLU | A | 188 | −22.367 | −5.628 | 3.607 | 1.00 | 16.77 |
| 45 | CA | GLU | A | 188 | −21.526 | −6.118 | 4.689 | 1.00 | 19.26 |
| 46 | CB | GLU | A | 188 | −22.175 | −7.317 | 5.353 | 1.00 | 21.32 |
| 47 | CG | GLU | A | 188 | −23.398 | −6.845 | 6.134 | 1.00 | 28.68 |
| 48 | CD | GLU | A | 188 | −24.275 | −7.966 | 6.623 | 1.00 | 30.83 |
| 49 | OE1 | GLU | A | 188 | −25.360 | −8.187 | 6.035 | 1.00 | 32.28 |
| 50 | OE2 | GLU | A | 188 | −23.887 | −8.615 | 7.611 | 1.00 | 34.12 |
| 51 | C | GLU | A | 188 | −20.061 | −6.366 | 4.321 | 1.00 | 18.83 |
| 52 | O | GLU | A | 188 | −19.174 | −6.172 | 5.147 | 1.00 | 18.44 |
| 53 | N | LEU | A | 189 | −19.805 | −6.729 | 3.069 | 1.00 | 17.42 |
| 54 | CA | LEU | A | 189 | −18.453 | −6.953 | 2.591 | 1.00 | 16.55 |
| 55 | CB | LEU | A | 189 | −18.512 | −7.441 | 1.148 | 1.00 | 16.21 |
| 56 | CG | LEU | A | 189 | −17.453 | −8.406 | 0.612 | 1.00 | 17.60 |
| 57 | CD1 | LEU | A | 189 | −17.165 | −8.041 | −0.823 | 1.00 | 15.95 |
| 58 | CD2 | LEU | A | 189 | −16.205 | −8.340 | 1.414 | 1.00 | 14.51 |
| 59 | C | LEU | A | 189 | −17.728 | −5.593 | 2.656 | 1.00 | 16.84 |
| 60 | O | LEU | A | 189 | −16.558 | −5.513 | 3.017 | 1.00 | 17.45 |
| 61 | N | ILE | A | 190 | −18.449 | −4.535 | 2.305 | 1.00 | 16.26 |
| 62 | CA | ILE | A | 190 | −17.926 | −3.189 | 2.322 | 1.00 | 15.67 |
| 63 | CB | ILE | A | 190 | −18.964 | −2.176 | 1.775 | 1.00 | 14.53 |
| 64 | CG2 | ILE | A | 190 | −18.452 | −0.762 | 1.952 | 1.00 | 14.55 |
| 65 | CG1 | ILE | A | 190 | −19.203 | −2.438 | 0.289 | 1.00 | 14.16 |
| 66 | CD1 | ILE | A | 190 | −20.363 | −1.652 | −0.369 | 1.00 | 15.08 |
| 67 | C | ILE | A | 190 | −17.512 | −2.808 | 3.737 | 1.00 | 16.88 |
| 68 | O | ILE | A | 190 | −16.428 | −2.271 | 3.952 | 1.00 | 16.52 |
| 69 | N | THR | A | 191 | −18.379 | −3.090 | 4.703 | 1.00 | 18.22 |
| 70 | CA | THR | A | 191 | −18.115 | −2.803 | 6.108 | 1.00 | 18.80 |
| 71 | CB | THR | A | 191 | −19.328 | −3.082 | 6.930 | 1.00 | 17.79 |
| 72 | OG1 | THR | A | 191 | −20.333 | −2.147 | 6.560 | 1.00 | 20.38 |
| 73 | CG2 | THR | A | 191 | −19.024 | −2.939 | 8.390 | 1.00 | 18.36 |
| 74 | C | THR | A | 191 | −16.963 | −3.623 | 6.653 | 1.00 | 20.64 |
| 75 | O | THR | A | 191 | −16.101 | −3.083 | 7.337 | 1.00 | 22.47 |
| 76 | N | LYS | A | 192 | −16.948 | −4.918 | 6.360 | 1.00 | 19.94 |
| 77 | CA | LYS | A | 192 | −15.875 | −5.774 | 6.816 | 1.00 | 20.28 |
| 78 | CB | LYS | A | 192 | −16.162 | −7.223 | 6.451 | 1.00 | 22.80 |
| 79 | CG | LYS | A | 192 | −17.174 | −7.832 | 7.439 | 1.00 | 26.17 |
| 80 | CD | LYS | A | 192 | −17.835 | −9.116 | 6.972 | 1.00 | 27.88 |
| 81 | CE | LYS | A | 192 | −18.790 | −9.613 | 8.072 | 1.00 | 29.77 |
| 82 | NZ | LYS | A | 192 | −20.058 | −10.190 | 7.537 | 1.00 | 32.37 |
| 83 | C | LYS | A | 192 | −14.532 | −5.312 | 6.288 | 1.00 | 20.29 |
| 84 | O | LYS | A | 192 | −13.644 | −4.992 | 7.064 | 1.00 | 21.51 |
| 85 | N | VAL | A | 193 | −14.395 | −5.201 | 4.976 | 1.00 | 20.89 |
| 86 | CA | VAL | A | 193 | −13.142 | −4.743 | 4.392 | 1.00 | 19.43 |
| 87 | CB | VAL | A | 193 | −13.254 | −4.626 | 2.876 | 1.00 | 20.16 |
| 88 | CG1 | VAL | A | 193 | −12.043 | −3.930 | 2.312 | 1.00 | 20.33 |
| 89 | CG2 | VAL | A | 193 | −13.402 | −6.003 | 2.265 | 1.00 | 16.58 |
| 90 | C | VAL | A | 193 | −12.748 | −3.400 | 4.988 | 1.00 | 19.77 |
| 91 | O | VAL | A | 193 | −11.640 | −3.230 | 5.459 | 1.00 | 20.14 |
| 92 | N | SER | A | 194 | −13.684 | −2.464 | 5.018 | 1.00 | 21.53 |
| 93 | CA | SER | A | 194 | −13.438 | −1.124 | 5.560 | 1.00 | 21.04 |
| 94 | CB | SER | A | 194 | −14.738 | −0.328 | 5.609 | 1.00 | 19.63 |
| 95 | OG | SER | A | 194 | −14.496 | 0.998 | 6.016 | 1.00 | 19.69 |
| 96 | C | SER | A | 194 | −12.812 | −1.169 | 6.946 | 1.00 | 21.80 |
| 97 | O | SER | A | 194 | −11.778 | −0.549 | 7.187 | 1.00 | 21.17 |
| 98 | N | LYS | A | 195 | −13.448 | −1.919 | 7.840 | 1.00 | 22.99 |
| 99 | CA | LYS | A | 195 | −13.006 | −2.093 | 9.218 | 1.00 | 22.87 |
| 100 | CB | LYS | A | 195 | −14.048 | −2.881 | 10.015 | 1.00 | 23.64 |
| 101 | CG | LYS | A | 195 | −15.116 | −1.991 | 10.650 | 1.00 | 28.29 |
| 102 | CD | LYS | A | 195 | −15.080 | −0.578 | 10.043 | 1.00 | 30.25 |
| 103 | CE | LYS | A | 195 | −16.005 | 0.403 | 10.767 | 1.00 | 32.32 |
| 104 | NZ | LYS | A | 195 | −15.549 | 0.724 | 12.161 | 1.00 | 33.22 |
| 105 | C | LYS | A | 195 | −11.649 | −2.752 | 9.319 | 1.00 | 22.28 |
| 106 | O | LYS | A | 195 | −10.822 | −2.311 | 10.092 | 1.00 | 23.38 |
| 107 | N | ALA | A | 196 | −11.407 | −3.801 | 8.543 | 1.00 | 21.84 |
| 108 | CA | ALA | A | 196 | −10.115 | −4.464 | 8.587 | 1.00 | 21.79 |
| 109 | CB | ALA | A | 196 | −10.096 | −5.682 | 7.660 | 1.00 | 20.88 |
| 110 | C | ALA | A | 196 | −9.054 | −3.446 | 8.176 | 1.00 | 21.34 |
| 111 | O | ALA | A | 196 | −7.982 | −3.371 | 8.777 | 1.00 | 23.62 |
| 112 | N | HIS | A | 197 | −9.349 | −2.627 | 7.174 | 1.00 | 20.81 |
| 113 | CA | HIS | A | 197 | −8.377 | −1.624 | 6.762 | 1.00 | 20.38 |
| 114 | CB | HIS | A | 197 | −8.776 | −0.931 | 5.468 | 1.00 | 18.26 |
| 115 | CG | HIS | A | 197 | −7.852 | 0.177 | 5.088 | 1.00 | 19.16 |
| 116 | CD2 | HIS | A | 197 | −7.957 | 1.517 | 5.242 | 1.00 | 17.84 |
| 117 | ND1 | HIS | A | 197 | −6.634 | −0.043 | 4.478 | 1.00 | 19.65 |
| 118 | CE1 | HIS | A | 197 | −6.034 | 1.112 | 4.266 | 1.00 | 18.43 |
| 119 | NE2 | HIS | A | 197 | −6.817 | 2.073 | 4.718 | 1.00 | 20.25 |
| 120 | C | HIS | A | 197 | −8.152 | −0.585 | 7.871 | 1.00 | 21.21 |
| 121 | O | HIS | A | 197 | −7.013 | −0.296 | 8.243 | 1.00 | 22.59 |
| 122 | N | GLN | A | 198 | −9.224 | −0.037 | 8.419 | 1.00 | 21.25 |
| 123 | CA | GLN | A | 198 | −9.070 | 0.951 | 9.463 | 1.00 | 23.59 |
| 124 | CB | GLN | A | 198 | −10.427 | 1.387 | 9.948 | 1.00 | 26.42 |
| 125 | CG | GLN | A | 198 | −11.141 | 2.386 | 9.083 | 1.00 | 29.27 |
| 126 | CD | GLN | A | 198 | −12.553 | 2.537 | 9.568 | 1.00 | 30.05 |
| 127 | OE1 | GLN | A | 198 | −12.820 | 2.362 | 10.762 | 1.00 | 31.98 |
| 128 | NE2 | GLN | A | 198 | −13.485 | 2.764 | 8.647 | 1.00 | 31.27 |
| 129 | C | GLN | A | 198 | −8.275 | 0.423 | 10.653 | 1.00 | 24.07 |
| 130 | O | GLN | A | 198 | −7.373 | 1.090 | 11.170 | 1.00 | 25.03 |
| 131 | N | GLU | A | 199 | −8.633 | −0.777 | 11.089 | 1.00 | 25.01 |
| 132 | CA | GLU | A | 199 | −7.991 | −1.437 | 12.214 | 1.00 | 24.53 |
| 133 | CB | GLU | A | 199 | −8.771 | −2.675 | 12.599 | 1.00 | 24.38 |
| 134 | CG | GLU | A | 199 | −10.086 | −2.311 | 13.229 | 1.00 | 27.91 |
| 135 | CD | GLU | A | 199 | −10.859 | −3.511 | 13.707 | 1.00 | 29.06 |
| 136 | OE1 | GLU | A | 199 | −12.107 | −3.480 | 13.615 | 1.00 | 32.53 |
| 137 | OE2 | GLU | A | 199 | −10.225 | −4.477 | 14.194 | 1.00 | 30.26 |
| 138 | C | GLU | A | 199 | −6.539 | −1.784 | 12.002 | 1.00 | 23.37 |
| 139 | O | GLU | A | 199 | −5.807 | −1.883 | 12.963 | 1.00 | 24.39 |
| 140 | N | THR | A | 200 | −6.114 | −1.949 | 10.756 | 1.00 | 21.99 |
| 141 | CA | THR | A | 200 | −4.719 | −2.276 | 10.462 | 1.00 | 20.58 |
| 142 | CB | THR | A | 200 | −4.624 | −3.474 | 9.488 | 1.00 | 20.42 |
| 143 | OG1 | THR | A | 200 | −5.031 | −3.059 | 8.169 | 1.00 | 17.20 |
| 144 | CG2 | THR | A | 200 | −5.515 | −4.633 | 9.999 | 1.00 | 18.24 |
| 145 | C | THR | A | 200 | −3.942 | −1.086 | 9.872 | 1.00 | 19.62 |
| 146 | O | THR | A | 200 | −2.761 | −1.202 | 9.541 | 1.00 | 17.71 |
| 147 | N | PHE | A | 201 | −4.614 | 0.045 | 9.704 | 1.00 | 20.00 |
| 148 | CA | PHE | A | 201 | −3.958 | 1.220 | 9.161 | 1.00 | 21.05 |
| 149 | CB | PHE | A | 201 | −3.941 | 1.160 | 7.648 | 1.00 | 22.20 |
| 150 | CG | PHE | A | 201 | −2.984 | 2.112 | 7.024 | 1.00 | 21.15 |
| 151 | CD1 | PHE | A | 201 | −3.430 | 3.109 | 6.187 | 1.00 | 23.24 |
| 152 | CD2 | PHE | A | 201 | −1.613 | 1.964 | 7.228 | 1.00 | 24.07 |
| 153 | CE1 | PHE | A | 201 | −2.532 | 3.946 | 5.546 | 1.00 | 21.82 |
| 154 | CE2 | PHE | A | 201 | −0.703 | 2.792 | 6.593 | 1.00 | 23.53 |
| 155 | CZ | PHE | A | 201 | −1.161 | 3.781 | 5.751 | 1.00 | 22.68 |
| 156 | C | PHE | A | 201 | −4.619 | 2.521 | 9.560 | 1.00 | 21.47 |
| 157 | O | PHE | A | 201 | −5.549 | 2.962 | 8.906 | 1.00 | 22.07 |
| 158 | N | PRO | A | 202 | −4.089 | 3.193 | 10.586 | 1.00 | 23.56 |
| 159 | CD | PRO | A | 202 | −3.018 | 2.664 | 11.445 | 1.00 | 24.92 |
| 160 | CA | PRO | A | 202 | −4.580 | 4.468 | 11.116 | 1.00 | 23.81 |
| 161 | CB | PRO | A | 202 | −3.570 | 4.784 | 12.206 | 1.00 | 23.88 |
| 162 | CG | PRO | A | 202 | −3.216 | 3.465 | 12.713 | 1.00 | 25.50 |
| 163 | C | PRO | A | 202 | −4.548 | 5.554 | 10.064 | 1.00 | 24.15 |
| 164 | O | PRO | A | 202 | −3.584 | 5.647 | 9.294 | 1.00 | 23.02 |
| 165 | N | SER | A | 203 | −5.588 | 6.381 | 10.028 | 1.00 | 25.14 |
| 166 | CA | SER | A | 203 | −5.629 | 7.466 | 9.064 | 1.00 | 26.45 |
| 167 | CB | SER | A | 203 | −7.057 | 7.981 | 8.836 | 1.00 | 26.50 |
| 168 | OG | SER | A | 203 | −7.803 | 8.001 | 10.039 | 1.00 | 29.67 |
| 169 | C | SER | A | 203 | −4.712 | 8.551 | 9.594 | 1.00 | 25.55 |
| 170 | O | SER | A | 203 | −4.392 | 8.573 | 10.779 | 1.00 | 25.33 |
| 171 | N | LEU | A | 204 | −4.238 | 9.405 | 8.698 | 1.00 | 26.68 |
| 172 | CA | LEU | A | 204 | −3.326 | 10.483 | 9.056 | 1.00 | 28.37 |
| 173 | CB | LEU | A | 204 | −3.110 | 11.361 | 7.828 | 1.00 | 27.72 |
| 174 | CG | LEU | A | 204 | −1.948 | 12.342 | 7.757 | 1.00 | 27.81 |
| 175 | CD1 | LEU | A | 204 | −0.725 | 11.797 | 8.433 | 1.00 | 26.92 |
| 176 | CD2 | LEU | A | 204 | −1.676 | 12.614 | 6.300 | 1.00 | 27.54 |
| 177 | C | LEU | A | 204 | −3.841 | 11.302 | 10.241 | 1.00 | 29.51 |
| 178 | O | LEU | A | 204 | −3.105 | 11.590 | 11.187 | 1.00 | 30.16 |
| 179 | N | CYS | A | 205 | −5.142 | 11.561 | 10.216 | 1.00 | 31.31 |
| 180 | CA | CYS | A | 205 | −5.849 | 12.341 | 11.218 | 1.00 | 33.60 |
| 181 | CB | CYS | A | 205 | −7.301 | 12.480 | 10.791 | 1.00 | 35.57 |
| 182 | SG | CYS | A | 205 | −7.559 | 11.823 | 9.119 | 1.00 | 41.57 |
| 183 | C | CYS | A | 205 | −5.798 | 11.776 | 12.625 | 1.00 | 33.68 |
| 184 | O | CYS | A | 205 | −5.892 | 12.523 | 13.607 | 1.00 | 34.82 |
| 185 | N | GLN | A | 206 | −5.690 | 10.459 | 12.730 | 1.00 | 33.19 |
| 186 | CA | GLN | A | 206 | −5.642 | 9.804 | 14.030 | 1.00 | 32.16 |
| 187 | CB | GLN | A | 206 | −6.136 | 8.359 | 13.887 | 1.00 | 33.06 |
| 188 | CG | GLN | A | 206 | −5.908 | 7.486 | 15.107 | 1.00 | 32.99 |
| 189 | CD | GLN | A | 206 | −6.222 | 6.034 | 14.849 | 1.00 | 34.01 |
| 190 | OE1 | GLN | A | 206 | −6.813 | 5.686 | 13.821 | 1.00 | 33.58 |
| 191 | NE2 | GLN | A | 206 | −5.827 | 5.168 | 15.782 | 1.00 | 34.48 |
| 192 | C | GLN | A | 206 | −4.217 | 9.784 | 14.562 | 1.00 | 31.63 |
| 193 | O | GLN | A | 206 | −3.975 | 9.430 | 15.719 | 1.00 | 32.61 |
| 194 | N | LEU | A | 207 | −3.262 | 10.186 | 13.742 | 1.00 | 29.82 |
| 195 | CA | LEU | A | 207 | −1.898 | 10.088 | 14.192 | 1.00 | 28.12 |
| 196 | CB | LEU | A | 207 | −0.999 | 9.600 | 13.054 | 1.00 | 27.92 |
| 197 | CG | LEU | A | 207 | −0.628 | 8.105 | 13.010 | 1.00 | 27.55 |
| 198 | CD1 | LEU | A | 207 | −1.631 | 7.215 | 13.731 | 1.00 | 27.18 |
| 199 | CD2 | LEU | A | 207 | −0.484 | 7.679 | 11.557 | 1.00 | 25.94 |
| 200 | C | LEU | A | 207 | −1.281 | 11.251 | 14.921 | 1.00 | 26.93 |
| 201 | O | LEU | A | 207 | −1.502 | 12.423 | 14.590 | 1.00 | 26.30 |
| 202 | N | GLY | A | 208 | −0.609 | 10.893 | 16.010 | 1.00 | 25.66 |
| 203 | CA | GLY | A | 208 | 0.110 | 11.870 | 16.790 | 1.00 | 24.28 |
| 204 | C | GLY | A | 208 | 1.398 | 12.007 | 15.992 | 1.00 | 22.29 |
| 205 | O | GLY | A | 208 | 2.269 | 11.135 | 16.061 | 1.00 | 20.48 |
| 206 | N | LYS | A | 209 | 1.448 | 13.015 | 15.131 | 1.00 | 21.40 |
| 207 | CA | LYS | A | 209 | 2.605 | 13.238 | 14.296 | 1.00 | 21.36 |
| 208 | CB | LYS | A | 209 | 2.265 | 14.194 | 13.160 | 1.00 | 22.61 |
| 209 | CG | LYS | A | 209 | 1.457 | 13.511 | 12.069 | 1.00 | 25.95 |
| 210 | CD | LYS | A | 209 | 0.684 | 14.484 | 11.192 | 1.00 | 26.65 |
| 211 | CE | LYS | A | 209 | 1.552 | 15.151 | 10.148 | 1.00 | 27.62 |
| 212 | NZ | LYS | A | 209 | 0.683 | 15.849 | 9.151 | 1.00 | 27.27 |
| 213 | C | LYS | A | 209 | 3.783 | 13.742 | 15.076 | 1.00 | 20.75 |
| 214 | O | LYS | A | 209 | 3.620 | 14.478 | 16.042 | 1.00 | 22.12 |
| 215 | N | TYR | A | 210 | 4.955 | 13.210 | 14.747 | 1.00 | 20.02 |
| 216 | CA | TYR | A | 210 | 6.192 | 13.647 | 15.370 | 1.00 | 20.49 |
| 217 | CB | TYR | A | 210 | 6.534 | 12.837 | 16.629 | 1.00 | 18.47 |
| 218 | CG | TYR | A | 210 | 7.130 | 11.494 | 16.353 | 1.00 | 18.13 |
| 219 | CD1 | TYR | A | 210 | 8.496 | 11.358 | 16.171 | 1.00 | 15.36 |
| 220 | CE1 | TYR | A | 210 | 9.045 | 10.142 | 15.867 | 1.00 | 16.09 |
| 221 | CD2 | TYR | A | 210 | 6.325 | 10.369 | 16.229 | 1.00 | 16.18 |
| 222 | CE2 | TYR | A | 210 | 6.866 | 9.144 | 15.925 | 1.00 | 15.25 |
| 223 | CZ | TYR | A | 210 | 8.229 | 9.037 | 15.744 | 1.00 | 15.38 |
| 224 | OH | TYR | A | 210 | 8.797 | 7.822 | 15.453 | 1.00 | 16.80 |
| 225 | C | TYR | A | 210 | 7.263 | 13.591 | 14.283 | 1.00 | 19.53 |
| 226 | O | TYR | A | 210 | 7.099 | 12.927 | 13.266 | 1.00 | 19.97 |
| 227 | N | THR | A | 211 | 8.332 | 14.335 | 14.483 | 1.00 | 20.32 |
| 228 | CA | THR | A | 211 | 9.392 | 14.441 | 13.504 | 1.00 | 19.85 |
| 229 | CB | THR | A | 211 | 9.434 | 15.910 | 13.061 | 1.00 | 20.41 |
| 230 | OG1 | THR | A | 211 | 9.454 | 16.758 | 14.222 | 1.00 | 22.41 |
| 231 | CG2 | THR | A | 211 | 8.177 | 16.261 | 12.291 | 1.00 | 22.14 |
| 232 | C | THR | A | 211 | 10.781 | 13.998 | 13.982 | 1.00 | 20.02 |
| 233 | O | THR | A | 211 | 10.945 | 13.465 | 15.077 | 1.00 | 22.75 |
| 234 | N | THR | A | 212 | 11.774 | 14.147 | 13.117 | 1.00 | 20.00 |
| 235 | CA | THR | A | 212 | 13.150 | 13.829 | 13.447 | 1.00 | 19.89 |
| 236 | CB | THR | A | 212 | 13.567 | 12.417 | 12.996 | 1.00 | 20.40 |
| 237 | OG1 | THR | A | 212 | 14.895 | 12.157 | 13.451 | 1.00 | 19.09 |
| 238 | CG2 | THR | A | 212 | 13.562 | 12.268 | 11.467 | 1.00 | 18.30 |
| 239 | C | THR | A | 212 | 14.001 | 14.876 | 12.737 | 1.00 | 22.16 |
| 240 | O | THR | A | 212 | 13.544 | 15.506 | 11.771 | 1.00 | 20.36 |
| 241 | N | ASN | A | 213 | 15.201 | 15.139 | 13.261 | 1.00 | 24.37 |
| 242 | CA | ASN | A | 213 | 16.080 | 16.104 | 12.608 | 1.00 | 24.35 |
| 243 | CB | ASN | A | 213 | 16.749 | 17.125 | 13.577 | 1.00 | 24.35 |
| 244 | CG | ASN | A | 213 | 17.424 | 16.484 | 14.788 | 1.00 | 23.76 |
| 245 | OD1 | ASN | A | 213 | 17.681 | 15.305 | 14.810 | 1.00 | 24.80 |
| 246 | ND2 | ASN | A | 213 | 17.707 | 17.294 | 15.794 | 1.00 | 22.88 |
| 247 | C | ASN | A | 213 | 17.083 | 15.434 | 11.688 | 1.00 | 25.10 |
| 248 | O | ASN | A | 213 | 17.747 | 16.090 | 10.890 | 1.00 | 26.32 |
| 249 | N | SER | A | 214 | 17.114 | 14.106 | 11.704 | 1.00 | 24.69 |
| 250 | CA | SER | A | 214 | 18.037 | 13.377 | 10.832 | 1.00 | 25.17 |
| 251 | CB | SER | A | 214 | 17.831 | 11.882 | 10.948 | 1.00 | 25.27 |
| 252 | OG | SER | A | 214 | 18.257 | 11.442 | 12.211 | 1.00 | 25.70 |
| 253 | C | SER | A | 214 | 17.940 | 13.795 | 9.369 | 1.00 | 25.21 |
| 254 | O | SER | A | 214 | 16.844 | 13.925 | 8.821 | 1.00 | 24.04 |
| 255 | N | SER | A | 215 | 19.107 | 14.043 | 8.768 | 1.00 | 26.72 |
| 256 | CA | SER | A | 215 | 19.264 | 14.441 | 7.377 | 1.00 | 25.06 |
| 257 | CB | SER | A | 215 | 19.734 | 13.237 | 6.595 | 1.00 | 23.52 |
| 258 | OG | SER | A | 215 | 20.845 | 12.696 | 7.296 | 1.00 | 27.19 |
| 259 | C | SER | A | 215 | 18.040 | 15.082 | 6.756 | 1.00 | 24.94 |
| 260 | O | SER | A | 215 | 17.514 | 14.611 | 5.759 | 1.00 | 26.62 |
| 261 | N | ALA | A | 216 | 17.527 | 16.084 | 7.451 | 1.00 | 26.24 |
| 262 | CA | ALA | A | 216 | 16.395 | 16.875 | 7.004 | 1.00 | 27.32 |
| 263 | CB | ALA | A | 216 | 15.686 | 17.514 | 8.207 | 1.00 | 24.42 |
| 264 | C | ALA | A | 216 | 17.012 | 17.930 | 6.092 | 1.00 | 28.48 |
| 265 | O | ALA | A | 216 | 16.372 | 18.427 | 5.159 | 1.00 | 29.23 |
| 266 | N | ASP | A | 217 | 18.308 | 18.158 | 6.324 | 1.00 | 28.95 |
| 267 | CA | ASP | A | 217 | 19.169 | 19.109 | 5.620 | 1.00 | 30.21 |
| 268 | CB | ASP | A | 217 | 20.647 | 18.851 | 6.056 | 1.00 | 32.59 |
| 269 | CG | ASP | A | 217 | 21.619 | 20.009 | 5.694 | 1.00 | 33.99 |
| 270 | OD1 | ASP | A | 217 | 22.382 | 20.458 | 6.603 | 1.00 | 37.14 |
| 271 | OD2 | ASP | A | 217 | 21.678 | 20.452 | 4.519 | 1.00 | 33.28 |
| 272 | C | ASP | A | 217 | 19.046 | 18.975 | 4.096 | 1.00 | 28.62 |
| 273 | O | ASP | A | 217 | 18.423 | 19.813 | 3.430 | 1.00 | 27.65 |
| 274 | N | HIS | A | 218 | 19.684 | 17.936 | 3.555 | 1.00 | 27.53 |
| 275 | CA | HIS | A | 218 | 19.707 | 17.691 | 2.116 | 1.00 | 25.97 |
| 276 | CB | HIS | A | 218 | 20.895 | 18.410 | 1.486 | 1.00 | 28.45 |
| 277 | CG | HIS | A | 218 | 22.207 | 17.735 | 1.741 | 1.00 | 29.85 |
| 278 | CD2 | HIS | A | 218 | 23.107 | 17.186 | 0.889 | 1.00 | 31.13 |
| 279 | ND1 | HIS | A | 218 | 22.711 | 17.529 | 3.012 | 1.00 | 30.86 |
| 280 | CE1 | HIS | A | 218 | 23.860 | 16.886 | 2.931 | 1.00 | 31.67 |
| 281 | NE2 | HIS | A | 218 | 24.125 | 16.665 | 1.654 | 1.00 | 32.39 |
| 282 | C | HIS | A | 218 | 19.854 | 16.204 | 1.859 | 1.00 | 23.88 |
| 283 | O | HIS | A | 218 | 20.188 | 15.445 | 2.766 | 1.00 | 25.08 |
| 284 | N | ARG | A | 219 | 19.741 | 15.824 | 0.599 | 1.00 | 22.00 |
| 285 | CA | ARG | A | 219 | 19.825 | 14.435 | 0.198 | 1.00 | 22.41 |
| 286 | CB | ARG | A | 219 | 19.159 | 14.261 | −1.166 | 1.00 | 22.50 |
| 287 | CG | ARG | A | 219 | 17.737 | 14.801 | −1.236 | 1.00 | 22.91 |
| 288 | CD | ARG | A | 219 | 17.098 | 14.440 | −2.571 | 1.00 | 22.98 |
| 289 | NE | ARG | A | 219 | 15.802 | 15.093 | −2.756 | 1.00 | 22.05 |
| 290 | CZ | ARG | A | 219 | 14.916 | 14.760 | −3.693 | 1.00 | 19.87 |
| 291 | NH1 | ARG | A | 219 | 15.158 | 13.768 | −4.544 | 1.00 | 17.44 |
| 292 | NH2 | ARG | A | 219 | 13.787 | 15.440 | −3.792 | 1.00 | 20.09 |
| 293 | C | ARG | A | 219 | 21.225 | 13.818 | 0.163 | 1.00 | 22.78 |
| 294 | O | ARG | A | 219 | 22.165 | 14.403 | −0.377 | 1.00 | 22.77 |
| 295 | N | VAL | A | 220 | 21.352 | 12.643 | 0.766 | 1.00 | 22.75 |
| 296 | CA | VAL | A | 220 | 22.603 | 11.898 | 0.779 | 1.00 | 22.59 |
| 297 | CB | VAL | A | 220 | 23.189 | 11.750 | 2.201 | 1.00 | 22.90 |
| 298 | CG1 | VAL | A | 220 | 23.673 | 13.119 | 2.722 | 1.00 | 24.17 |
| 299 | CG2 | VAL | A | 220 | 22.166 | 11.134 | 3.164 | 1.00 | 20.45 |
| 300 | C | VAL | A | 220 | 22.235 | 10.539 | 0.212 | 1.00 | 24.31 |
| 301 | O | VAL | A | 220 | 21.043 | 10.247 | 0.048 | 1.00 | 26.77 |
| 302 | N | GLN | A | 221 | 23.222 | 9.718 | −0.126 | 1.00 | 23.99 |
| 303 | CA | GLN | A | 221 | 22.932 | 8.402 | −0.680 | 1.00 | 24.25 |
| 304 | CB | GLN | A | 221 | 24.230 | 7.620 | −0.944 | 1.00 | 25.91 |
| 305 | CG | GLN | A | 221 | 24.002 | 6.223 | −1.582 | 1.00 | 29.12 |
| 306 | CD | GLN | A | 221 | 25.250 | 5.300 | −1.593 | 1.00 | 31.49 |
| 307 | OE1 | GLN | A | 221 | 26.142 | 5.465 | −2.421 | 1.00 | 34.56 |
| 308 | NE2 | GLN | A | 221 | 25.276 | 4.299 | −0.706 | 1.00 | 31.02 |
| 309 | C | GLN | A | 221 | 22.011 | 7.567 | 0.232 | 1.00 | 24.22 |
| 310 | O | GLN | A | 221 | 21.099 | 6.903 | −0.254 | 1.00 | 25.20 |
| 311 | N | LEU | A | 222 | 22.242 | 7.614 | 1.544 | 1.00 | 23.14 |
| 312 | CA | LEU | A | 222 | 21.464 | 6.814 | 2.477 | 1.00 | 22.34 |
| 313 | CB | LEU | A | 222 | 21.858 | 5.351 | 2.255 | 1.00 | 20.04 |
| 314 | CG | LEU | A | 222 | 21.047 | 4.138 | 2.682 | 1.00 | 20.69 |
| 315 | CD1 | LEU | A | 222 | 21.290 | 3.727 | 4.100 | 1.00 | 20.10 |
| 316 | CD2 | LEU | A | 222 | 19.596 | 4.438 | 2.430 | 1.00 | 21.58 |
| 317 | C | LEU | A | 222 | 21.788 | 7.197 | 3.921 | 1.00 | 22.91 |
| 318 | O | LEU | A | 222 | 22.967 | 7.309 | 4.290 | 1.00 | 24.38 |
| 319 | N | ASP | A | 223 | 20.755 | 7.418 | 4.735 | 1.00 | 22.66 |
| 320 | CA | ASP | A | 223 | 20.938 | 7.726 | 6.162 | 1.00 | 21.34 |
| 321 | CB | ASP | A | 223 | 20.079 | 8.925 | 6.610 | 1.00 | 22.74 |
| 322 | CG | ASP | A | 223 | 20.221 | 9.238 | 8.126 | 1.00 | 25.18 |
| 323 | OD1 | ASP | A | 223 | 20.458 | 10.412 | 8.508 | 1.00 | 26.69 |
| 324 | OD2 | ASP | A | 223 | 20.059 | 8.320 | 8.955 | 1.00 | 26.37 |
| 325 | C | ASP | A | 223 | 20.531 | 6.443 | 6.889 | 1.00 | 20.74 |
| 326 | O | ASP | A | 223 | 19.359 | 6.054 | 6.915 | 1.00 | 18.76 |
| 327 | N | LEU | A | 224 | 21.518 | 5.782 | 7.463 | 1.00 | 20.18 |
| 328 | CA | LEU | A | 224 | 21.303 | 4.524 | 8.145 | 1.00 | 19.89 |
| 329 | CB | LEU | A | 224 | 22.632 | 3.915 | 8.529 | 1.00 | 21.07 |
| 330 | CG | LEU | A | 224 | 23.322 | 3.093 | 7.463 | 1.00 | 22.72 |
| 331 | CD1 | LEU | A | 224 | 24.607 | 2.566 | 8.072 | 1.00 | 20.82 |
| 332 | CD2 | LEU | A | 224 | 22.418 | 1.935 | 7.050 | 1.00 | 22.65 |
| 333 | C | LEU | A | 224 | 20.411 | 4.607 | 9.358 | 1.00 | 19.76 |
| 334 | O | LEU | A | 224 | 19.666 | 3.681 | 9.635 | 1.00 | 20.96 |
| 335 | N | GLY | A | 225 | 20.524 | 5.679 | 10.118 | 1.00 | 17.05 |
| 336 | CA | GLY | A | 225 | 19.673 | 5.807 | 11.268 | 1.00 | 17.01 |
| 337 | C | GLY | A | 225 | 18.243 | 5.835 | 10.775 | 1.00 | 17.46 |
| 338 | O | GLY | A | 225 | 17.377 | 5.228 | 11.381 | 1.00 | 16.42 |
| 339 | N | LEU | A | 226 | 18.025 | 6.481 | 9.628 | 1.00 | 18.64 |
| 340 | CA | LEU | A | 226 | 16.699 | 6.608 | 9.012 | 1.00 | 18.78 |
| 341 | CB | LEU | A | 226 | 16.707 | 7.762 | 8.003 | 1.00 | 18.63 |
| 342 | CG | LEU | A | 226 | 15.798 | 9.002 | 8.195 | 1.00 | 19.85 |
| 343 | CD1 | LEU | A | 226 | 15.605 | 9.392 | 9.643 | 1.00 | 16.64 |
| 344 | CD2 | LEU | A | 226 | 16.337 | 10.201 | 7.380 | 1.00 | 17.14 |
| 345 | C | LEU | A | 226 | 16.231 | 5.295 | 8.359 | 1.00 | 18.28 |
| 346 | O | LEU | A | 226 | 15.073 | 4.886 | 8.530 | 1.00 | 16.45 |
| 347 | N | TRP | A | 227 | 17.124 | 4.624 | 7.634 | 1.00 | 17.71 |
| 348 | CA | TRP | A | 227 | 16.776 | 3.352 | 7.015 | 1.00 | 18.68 |
| 349 | CB | TRP | A | 227 | 17.926 | 2.775 | 6.206 | 1.00 | 17.57 |
| 350 | CG | TRP | A | 227 | 17.835 | 1.262 | 6.070 | 1.00 | 16.59 |
| 351 | CD2 | TRP | A | 227 | 16.919 | 0.501 | 5.253 | 1.00 | 15.32 |
| 352 | CE2 | TRP | A | 227 | 17.228 | −0.860 | 5.433 | 1.00 | 13.42 |
| 353 | CE3 | TRP | A | 227 | 15.866 | 0.838 | 4.392 | 1.00 | 17.17 |
| 354 | CD1 | TRP | A | 227 | 18.624 | 0.350 | 6.695 | 1.00 | 16.34 |
| 355 | NE1 | TRP | A | 227 | 18.270 | −0.926 | 6.316 | 1.00 | 15.17 |
| 356 | CZ2 | TRP | A | 227 | 16.529 | −1.882 | 4.786 | 1.00 | 13.09 |
| 357 | CZ3 | TRP | A | 227 | 15.168 | −0.186 | 3.747 | 1.00 | 15.40 |
| 358 | CH2 | TRP | A | 227 | 15.505 | −1.521 | 3.950 | 1.00 | 14.59 |
| 359 | C | TRP | A | 227 | 16.407 | 2.364 | 8.122 | 1.00 | 20.52 |
| 360 | O | TRP | A | 227 | 15.585 | 1.465 | 7.917 | 1.00 | 20.81 |
| 361 | N | ASP | A | 228 | 17.049 | 2.511 | 9.281 | 1.00 | 21.28 |
| 362 | CA | ASP | A | 228 | 16.772 | 1.649 | 10.418 | 1.00 | 21.73 |
| 363 | CB | ASP | A | 228 | 17.773 | 1.893 | 11.512 | 1.00 | 25.14 |
| 364 | CG | ASP | A | 228 | 18.798 | 0.815 | 11.570 | 1.00 | 28.26 |
| 365 | OD1 | ASP | A | 228 | 19.686 | 0.779 | 10.668 | 1.00 | 29.32 |
| 366 | OD2 | ASP | A | 228 | 18.682 | −0.012 | 12.504 | 1.00 | 31.33 |
| 367 | C | ASP | A | 228 | 15.379 | 1.823 | 10.970 | 1.00 | 20.87 |
| 368 | O | ASP | A | 228 | 14.695 | 0.851 | 11.249 | 1.00 | 22.70 |
| 369 | N | LYS | A | 229 | 14.982 | 3.062 | 11.192 | 1.00 | 20.53 |
| 370 | CA | LYS | A | 229 | 13.640 | 3.354 | 11.691 | 1.00 | 19.84 |
| 371 | CB | LYS | A | 229 | 13.511 | 4.877 | 11.931 | 1.00 | 19.61 |
| 372 | CG | LYS | A | 229 | 12.162 | 5.331 | 12.479 | 1.00 | 23.06 |
| 373 | CD | LYS | A | 229 | 11.776 | 4.555 | 13.750 | 1.00 | 25.62 |
| 374 | CE | LYS | A | 229 | 10.401 | 4.940 | 14.260 | 1.00 | 25.11 |
| 375 | NZ | LYS | A | 229 | 9.907 | 3.921 | 15.249 | 1.00 | 28.39 |
| 376 | C | LYS | A | 229 | 12.643 | 2.889 | 10.614 | 1.00 | 18.28 |
| 377 | O | LYS | A | 229 | 11.662 | 2.210 | 10.902 | 1.00 | 17.81 |
| 378 | N | PHE | A | 230 | 13.016 | 3.159 | 9.362 | 1.00 | 19.38 |
| 379 | CA | PHE | A | 230 | 12.238 | 2.857 | 8.170 | 1.00 | 17.62 |
| 380 | CB | PHE | A | 230 | 12.937 | 3.454 | 6.939 | 1.00 | 16.93 |
| 381 | CG | PHE | A | 230 | 12.211 | 3.217 | 5.658 | 1.00 | 16.42 |
| 382 | CD1 | PHE | A | 230 | 11.020 | 3.878 | 5.388 | 1.00 | 17.12 |
| 383 | CD2 | PHE | A | 230 | 12.683 | 2.284 | 4.744 | 1.00 | 18.74 |
| 384 | CE1 | PHE | A | 230 | 10.296 | 3.611 | 4.226 | 1.00 | 18.18 |
| 385 | CE2 | PHE | A | 230 | 11.968 | 1.997 | 3.569 | 1.00 | 19.04 |
| 386 | CZ | PHE | A | 230 | 10.769 | 2.666 | 3.314 | 1.00 | 18.96 |
| 387 | C | PHE | A | 230 | 11.938 | 1.380 | 7.959 | 1.00 | 17.02 |
| 388 | O | PHE | A | 230 | 10.776 | 1.000 | 7.777 | 1.00 | 17.25 |
| 389 | N | SER | A | 231 | 12.971 | 0.551 | 7.992 | 1.00 | 17.74 |
| 390 | CA | SER | A | 231 | 12.810 | −0.882 | 7.788 | 1.00 | 18.46 |
| 391 | CB | SER | A | 231 | 14.172 | −1.563 | 7.651 | 1.00 | 18.00 |
| 392 | OG | SER | A | 231 | 15.005 | −1.275 | 8.765 | 1.00 | 18.22 |
| 393 | C | SER | A | 231 | 12.005 | −1.524 | 8.916 | 1.00 | 18.14 |
| 394 | O | SER | A | 231 | 11.374 | −2.565 | 8.711 | 1.00 | 19.28 |
| 395 | N | GLU | A | 232 | 12.071 | −0.931 | 10.109 | 1.00 | 18.65 |
| 396 | CA | GLU | A | 232 | 11.329 | −1.420 | 11.277 | 1.00 | 18.42 |
| 397 | CB | GLU | A | 232 | 11.766 | −0.685 | 12.541 | 1.00 | 21.44 |
| 398 | CG | GLU | A | 232 | 10.733 | −0.690 | 13.698 | 1.00 | 24.22 |
| 399 | CD | GLU | A | 232 | 11.158 | 0.204 | 14.866 | 1.00 | 27.21 |
| 400 | OE1 | GLU | A | 232 | 12.314 | 0.072 | 15.327 | 1.00 | 27.98 |
| 401 | OE2 | GLU | A | 232 | 10.347 | 1.045 | 15.318 | 1.00 | 29.80 |
| 402 | C | GLU | A | 232 | 9.857 | −1.149 | 11.035 | 1.00 | 16.09 |
| 403 | O | GLU | A | 232 | 9.031 | −2.026 | 11.211 | 1.00 | 14.77 |
| 404 | N | LEU | A | 233 | 9.555 | 0.087 | 10.648 | 1.00 | 13.93 |
| 405 | CA | LEU | A | 233 | 8.191 | 0.514 | 10.349 | 1.00 | 13.81 |
| 406 | CB | LEU | A | 233 | 8.124 | 2.031 | 10.109 | 1.00 | 12.25 |
| 407 | CG | LEU | A | 233 | 8.385 | 2.956 | 11.314 | 1.00 | 12.32 |
| 408 | CD1 | LEU | A | 233 | 8.406 | 4.374 | 10.841 | 1.00 | 12.38 |
| 409 | CD2 | LEU | A | 233 | 7.341 | 2.789 | 12.399 | 1.00 | 13.65 |
| 410 | C | LEU | A | 233 | 7.646 | −0.233 | 9.139 | 1.00 | 13.48 |
| 411 | O | LEU | A | 233 | 6.458 | −0.523 | 9.073 | 1.00 | 15.95 |
| 412 | N | ALA | A | 234 | 8.503 | −0.549 | 8.180 | 1.00 | 12.52 |
| 413 | CA | ALA | A | 234 | 8.049 | −1.284 | 7.016 | 1.00 | 14.87 |
| 414 | CB | ALA | A | 234 | 9.112 | −1.275 | 5.915 | 1.00 | 13.08 |
| 415 | C | ALA | A | 234 | 7.701 | −2.729 | 7.459 | 1.00 | 16.06 |
| 416 | O | ALA | A | 234 | 6.619 | −3.242 | 7.142 | 1.00 | 15.82 |
| 417 | N | THR | A | 235 | 8.578 | −3.351 | 8.243 | 1.00 | 15.16 |
| 418 | CA | THR | A | 235 | 8.334 | −4.707 | 8.738 | 1.00 | 15.64 |
| 419 | CB | THR | A | 235 | 9.540 | −5.213 | 9.619 | 1.00 | 17.73 |
| 420 | OG1 | THR | A | 235 | 10.662 | −5.493 | 8.774 | 1.00 | 17.62 |
| 421 | CG2 | THR | A | 235 | 9.198 | −6.473 | 10.377 | 1.00 | 18.14 |
| 422 | C | THR | A | 235 | 7.015 | −4.802 | 9.526 | 1.00 | 15.67 |
| 423 | O | THR | A | 235 | 6.244 | −5.750 | 9.341 | 1.00 | 16.12 |
| 424 | N | LYS | A | 236 | 6.761 | −3.838 | 10.400 | 1.00 | 14.40 |
| 425 | CA | LYS | A | 236 | 5.532 | −3.854 | 11.179 | 1.00 | 15.93 |
| 426 | CB | LYS | A | 236 | 5.548 | −2.778 | 12.264 | 1.00 | 16.01 |
| 427 | CG | LYS | A | 236 | 6.596 | −2.951 | 13.334 | 1.00 | 21.21 |
| 428 | CD | LYS | A | 236 | 6.412 | −1.894 | 14.419 | 1.00 | 23.81 |
| 429 | CE | LYS | A | 236 | 7.583 | −1.851 | 15.394 | 1.00 | 24.68 |
| 430 | NZ | LYS | A | 236 | 7.749 | −3.128 | 16.139 | 1.00 | 25.36 |
| 431 | C | LYS | A | 236 | 4.342 | −3.633 | 10.260 | 1.00 | 14.33 |
| 432 | O | LYS | A | 236 | 3.231 | −4.088 | 10.538 | 1.00 | 13.57 |
| 433 | N | CYS | A | 237 | 4.579 | −2.938 | 9.156 | 1.00 | 14.60 |
| 434 | CA | CYS | A | 237 | 3.519 | −2.681 | 8.217 | 1.00 | 16.39 |
| 435 | CB | CYS | A | 237 | 3.870 | −1.561 | 7.234 | 1.00 | 14.97 |
| 436 | SG | CYS | A | 237 | 2.335 | −0.904 | 6.483 | 1.00 | 17.49 |
| 437 | C | CYS | A | 237 | 3.158 | −3.979 | 7.489 | 1.00 | 16.47 |
| 438 | O | CYS | A | 237 | 1.984 | −4.327 | 7.360 | 1.00 | 17.24 |
| 439 | N | ILE | A | 238 | 4.170 | −4.722 | 7.054 | 1.00 | 15.33 |
| 440 | CA | ILE | A | 238 | 3.934 | −5.978 | 6.352 | 1.00 | 13.85 |
| 441 | CB | ILE | A | 238 | 5.264 | −6.601 | 5.999 | 1.00 | 13.01 |
| 442 | CG2 | ILE | A | 238 | 5.073 | −8.038 | 5.514 | 1.00 | 14.88 |
| 443 | CG1 | ILE | A | 238 | 5.978 | −5.715 | 4.969 | 1.00 | 12.70 |
| 444 | CD1 | ILE | A | 238 | 7.405 | −6.092 | 4.758 | 1.00 | 10.75 |
| 445 | C | ILE | A | 238 | 3.078 | −6.930 | 7.195 | 1.00 | 14.22 |
| 446 | O | ILE | A | 238 | 2.208 | −7.642 | 6.691 | 1.00 | 14.79 |
| 447 | N | ILE | A | 239 | 3.359 | −6.966 | 8.482 | 1.00 | 14.17 |
| 448 | CA | ILE | A | 239 | 2.609 | −7.794 | 9.414 | 1.00 | 14.19 |
| 449 | CB | ILE | A | 239 | 3.238 | −7.724 | 10.827 | 1.00 | 14.59 |
| 450 | CG2 | ILE | A | 239 | 2.230 | −8.068 | 11.870 | 1.00 | 15.96 |
| 451 | CG1 | ILE | A | 239 | 4.464 | −8.635 | 10.917 | 1.00 | 13.80 |
| 452 | CD1 | ILE | A | 239 | 5.315 | −8.368 | 12.155 | 1.00 | 13.34 |
| 453 | C | ILE | A | 239 | 1.168 | −7.274 | 9.443 | 1.00 | 14.54 |
| 454 | O | ILE | A | 239 | 0.226 | −8.057 | 9.452 | 1.00 | 14.16 |
| 455 | N | LYS | A | 240 | 0.988 | −5.962 | 9.419 | 1.00 | 15.39 |
| 456 | CA | LYS | A | 240 | −0.361 | −5.415 | 9.427 | 1.00 | 15.53 |
| 457 | CB | LYS | A | 240 | −0.350 | −3.917 | 9.717 | 1.00 | 18.27 |
| 458 | CG | LYS | A | 240 | −0.078 | −3.589 | 11.170 | 1.00 | 21.05 |
| 459 | CD | LYS | A | 240 | −1.336 | −3.699 | 12.002 | 1.00 | 23.72 |
| 460 | CE | LYS | A | 240 | −1.050 | −4.179 | 13.432 | 1.00 | 27.33 |
| 461 | NZ | LYS | A | 240 | −0.927 | −5.697 | 13.537 | 1.00 | 30.09 |
| 462 | C | LYS | A | 240 | −1.107 | −5.702 | 8.131 | 1.00 | 15.65 |
| 463 | O | LYS | A | 240 | −2.336 | −5.785 | 8.139 | 1.00 | 13.48 |
| 464 | N | ILE | A | 241 | −0.369 | −5.867 | 7.031 | 1.00 | 14.37 |
| 465 | CA | ILE | A | 241 | −0.977 | −6.171 | 5.739 | 1.00 | 15.71 |
| 466 | CB | ILE | A | 241 | −0.012 | −5.897 | 4.560 | 1.00 | 15.46 |
| 467 | CG2 | ILE | A | 241 | −0.540 | −6.514 | 3.275 | 1.00 | 17.68 |
| 468 | CG1 | ILE | A | 241 | 0.111 | −4.389 | 4.349 | 1.00 | 16.06 |
| 469 | CD1 | ILE | A | 241 | 1.192 | −3.933 | 3.377 | 1.00 | 17.03 |
| 470 | C | ILE | A | 241 | −1.450 | −7.630 | 5.746 | 1.00 | 16.21 |
| 471 | O | ILE | A | 241 | −2.542 | −7.950 | 5.259 | 1.00 | 15.85 |
| 472 | N | VAL | A | 242 | −0.631 | −8.505 | 6.332 | 1.00 | 17.19 |
| 473 | CA | VAL | A | 242 | −0.964 | −9.923 | 6.455 | 1.00 | 16.87 |
| 474 | CB | VAL | A | 242 | 0.180 | −10.726 | 7.071 | 1.00 | 15.52 |
| 475 | CG1 | VAL | A | 242 | −0.310 | −12.078 | 7.478 | 1.00 | 15.69 |
| 476 | CG2 | VAL | A | 242 | 1.319 | −10.848 | 6.068 | 1.00 | 16.20 |
| 477 | C | VAL | A | 242 | −2.213 | −10.049 | 7.316 | 1.00 | 16.56 |
| 478 | O | VAL | A | 242 | −3.130 | −10.786 | 6.968 | 1.00 | 16.69 |
| 479 | N | GLU | A | 243 | −2.269 | −9.291 | 8.407 | 1.00 | 16.45 |
| 480 | CA | GLU | A | 243 | −3.451 | −9.301 | 9.271 | 1.00 | 19.31 |
| 481 | CB | GLU | A | 243 | −3.273 | −8.371 | 10.468 | 1.00 | 22.56 |
| 482 | CG | GLU | A | 243 | −4.521 | −8.364 | 11.304 | 1.00 | 27.44 |
| 483 | CD | GLU | A | 243 | −4.599 | −7.233 | 12.284 | 1.00 | 31.18 |
| 484 | OE1 | GLU | A | 243 | −3.565 | −6.948 | 12.933 | 1.00 | 33.07 |
| 485 | OE2 | GLU | A | 243 | −5.711 | −6.649 | 12.418 | 1.00 | 33.67 |
| 486 | C | GLU | A | 243 | −4.697 | −8.839 | 8.500 | 1.00 | 17.64 |
| 487 | O | GLU | A | 243 | −5.775 | −9.350 | 8.703 | 1.00 | 14.49 |
| 488 | N | PHE | A | 244 | −4.527 | −7.799 | 7.686 | 1.00 | 18.29 |
| 489 | CA | PHE | A | 244 | −5.586 | −7.235 | 6.845 | 1.00 | 16.37 |
| 490 | CB | PHE | A | 244 | −5.061 | −6.002 | 6.093 | 1.00 | 17.51 |
| 491 | CG | PHE | A | 244 | −5.971 | −5.497 | 4.998 | 1.00 | 14.72 |
| 492 | CD1 | PHE | A | 244 | −5.638 | −5.690 | 3.666 | 1.00 | 15.96 |
| 493 | CD2 | PHE | A | 244 | −7.121 | −4.800 | 5.298 | 1.00 | 15.04 |
| 494 | CE1 | PHE | A | 244 | −6.439 | −5.191 | 2.643 | 1.00 | 16.27 |
| 495 | CE2 | PHE | A | 244 | −7.934 | −4.295 | 4.281 | 1.00 | 16.72 |
| 496 | CZ | PHE | A | 244 | −7.595 | −4.489 | 2.954 | 1.00 | 14.78 |
| 497 | C | PHE | A | 244 | −6.062 | −8.294 | 5.853 | 1.00 | 15.24 |
| 498 | O | PHE | A | 244 | −7.264 | −8.505 | 5.718 | 1.00 | 15.07 |
| 499 | N | ALA | A | 245 | −5.118 | −8.972 | 5.195 | 1.00 | 14.79 |
| 500 | CA | ALA | A | 245 | −5.437 | −10.034 | 4.226 | 1.00 | 14.77 |
| 501 | CB | ALA | A | 245 | −4.147 | −10.593 | 3.592 | 1.00 | 14.54 |
| 502 | C | ALA | A | 245 | −6.216 | −11.157 | 4.895 | 1.00 | 13.91 |
| 503 | O | ALA | A | 245 | −7.220 | −11.586 | 4.386 | 1.00 | 13.71 |
| 504 | N | LYS | A | 246 | −5.762 | −11.596 | 6.063 | 1.00 | 14.98 |
| 505 | CA | LYS | A | 246 | −6.421 | −12.668 | 6.808 | 1.00 | 16.59 |
| 506 | CB | LYS | A | 246 | −5.658 | −12.963 | 8.100 | 1.00 | 17.81 |
| 507 | CG | LYS | A | 246 | −4.274 | −13.575 | 7.925 | 1.00 | 20.05 |
| 508 | CD | LYS | A | 246 | −4.351 | −14.931 | 7.282 | 1.00 | 21.82 |
| 509 | CE | LYS | A | 246 | −3.225 | −15.824 | 7.778 | 1.00 | 23.30 |
| 510 | NZ | LYS | A | 246 | −3.422 | −17.231 | 7.280 | 1.00 | 26.39 |
| 511 | C | LYS | A | 246 | −7.867 | −12.341 | 7.184 | 1.00 | 17.03 |
| 512 | O | LYS | A | 246 | −8.634 | −13.238 | 7.522 | 1.00 | 16.72 |
| 513 | N | ARG | A | 247 | −8.209 | −11.054 | 7.186 | 1.00 | 17.90 |
| 514 | CA | ARG | A | 247 | −9.548 | −10.599 | 7.533 | 1.00 | 18.60 |
| 515 | CB | ARG | A | 247 | −9.443 | −9.373 | 8.421 | 1.00 | 20.73 |
| 516 | CG | ARG | A | 247 | −8.784 | −9.712 | 9.734 | 1.00 | 22.93 |
| 517 | CD | ARG | A | 247 | −8.272 | −8.510 | 10.443 | 1.00 | 24.91 |
| 518 | NE | ARG | A | 247 | −9.324 | −7.528 | 10.618 | 1.00 | 28.82 |
| 519 | CZ | ARG | A | 247 | −9.510 | −6.802 | 11.714 | 1.00 | 29.63 |
| 520 | NH1 | ARG | A | 247 | −8.726 | −6.940 | 12.772 | 1.00 | 28.78 |
| 521 | NH2 | ARG | A | 247 | −10.442 | −5.869 | 11.711 | 1.00 | 30.29 |
| 522 | C | ARG | A | 247 | −10.488 | −10.356 | 6.347 | 1.00 | 18.81 |
| 523 | O | ARG | A | 247 | −11.680 | −10.086 | 6.543 | 1.00 | 20.35 |
| 524 | N | LEU | A | 248 | −9.973 | −10.510 | 5.128 | 1.00 | 18.00 |
| 525 | CA | LEU | A | 248 | −10.775 | −10.339 | 3.931 | 1.00 | 17.53 |
| 526 | CB | LEU | A | 248 | −9.896 | −10.045 | 2.706 | 1.00 | 17.06 |
| 527 | CG | LEU | A | 248 | −9.054 | −8.760 | 2.591 | 1.00 | 15.80 |
| 528 | CD1 | LEU | A | 248 | −8.275 | −8.778 | 1.266 | 1.00 | 12.46 |
| 529 | CD2 | LEU | A | 248 | −9.927 | −7.506 | 2.710 | 1.00 | 14.81 |
| 530 | C | LEU | A | 248 | −11.534 | −11.646 | 3.713 | 1.00 | 18.26 |
| 531 | O | LEU | A | 248 | −10.965 | −12.729 | 3.824 | 1.00 | 17.01 |
| 532 | N | PRO | A | 249 | −12.843 | −11.567 | 3.419 | 1.00 | 18.73 |
| 533 | CD | PRO | A | 249 | −13.719 | −10.384 | 3.470 | 1.00 | 19.57 |
| 534 | CA | PRO | A | 249 | −13.633 | −12.779 | 3.200 | 1.00 | 19.22 |
| 535 | CB | PRO | A | 249 | −14.977 | −12.222 | 2.744 | 1.00 | 19.79 |
| 536 | CG | PRO | A | 249 | −15.099 | −11.016 | 3.621 | 1.00 | 20.52 |
| 537 | C | PRO | A | 249 | −13.053 | −13.756 | 2.197 | 1.00 | 19.27 |
| 538 | O | PRO | A | 249 | −12.726 | −13.387 | 1.075 | 1.00 | 20.55 |
| 539 | N | GLY | A | 250 | −12.903 | −15.004 | 2.641 | 1.00 | 20.87 |
| 540 | CA | GLY | A | 250 | −12.401 | −16.083 | 1.801 | 1.00 | 19.86 |
| 541 | C | GLY | A | 250 | −10.907 | −16.209 | 1.590 | 1.00 | 20.13 |
| 542 | O | GLY | A | 250 | −10.454 | −17.215 | 1.044 | 1.00 | 21.37 |
| 543 | N | PHE | A | 251 | −10.130 | −15.231 | 2.048 | 1.00 | 19.34 |
| 544 | CA | PHE | A | 251 | −8.672 | −15.257 | 1.861 | 1.00 | 19.32 |
| 545 | CB | PHE | A | 251 | −8.036 | −14.001 | 2.499 | 1.00 | 18.12 |
| 546 | CG | PHE | A | 251 | −6.559 | −13.903 | 2.297 | 1.00 | 17.10 |
| 547 | CD1 | PHE | A | 251 | −6.043 | −13.423 | 1.094 | 1.00 | 17.68 |
| 548 | CD2 | PHE | A | 251 | −5.682 | −14.368 | 3.277 | 1.00 | 15.40 |
| 549 | CE1 | PHE | A | 251 | −4.664 | −13.414 | 0.859 | 1.00 | 16.47 |
| 550 | CE2 | PHE | A | 251 | −4.324 | −14.365 | 3.060 | 1.00 | 16.00 |
| 551 | CZ | PHE | A | 251 | −3.804 | −13.891 | 1.850 | 1.00 | 17.36 |
| 552 | C | PHE | A | 251 | −8.157 | −16.518 | 2.535 | 1.00 | 18.63 |
| 553 | O | PHE | A | 251 | −7.576 | −17.418 | 1.921 | 1.00 | 18.89 |
| 554 | N | THR | A | 252 | −8.505 | −16.604 | 3.798 | 1.00 | 18.51 |
| 555 | CA | THR | A | 252 | −8.145 | −17.691 | 4.655 | 1.00 | 19.35 |
| 556 | CB | THR | A | 252 | −8.678 | −17.342 | 6.033 | 1.00 | 20.77 |
| 557 | OG1 | THR | A | 252 | −7.644 | −16.680 | 6.767 | 1.00 | 23.36 |
| 558 | CG2 | THR | A | 252 | −9.212 | −18.514 | 6.774 | 1.00 | 23.22 |
| 559 | C | THR | A | 252 | −8.634 | −19.033 | 4.132 | 1.00 | 19.84 |
| 560 | O | THR | A | 252 | −8.220 | −20.077 | 4.625 | 1.00 | 20.58 |
| 561 | N | GLY | A | 253 | −9.451 | −19.000 | 3.082 | 1.00 | 21.05 |
| 562 | CA | GLY | A | 253 | −9.980 | −20.224 | 2.500 | 1.00 | 20.89 |
| 563 | C | GLY | A | 253 | −9.123 | −20.789 | 1.383 | 1.00 | 21.46 |
| 564 | O | GLY | A | 253 | −9.376 | −21.899 | 0.890 | 1.00 | 21.33 |
| 565 | N | LEU | A | 254 | −8.135 | −20.011 | 0.948 | 1.00 | 20.37 |
| 566 | CA | LEU | A | 254 | −7.219 | −20.429 | −0.116 | 1.00 | 18.42 |
| 567 | CB | LEU | A | 254 | −6.552 | −19.180 | −0.708 | 1.00 | 20.47 |
| 568 | CG | LEU | A | 254 | −7.335 | −18.347 | −1.725 | 1.00 | 22.68 |
| 569 | CD1 | LEU | A | 254 | −8.777 | −18.338 | −1.352 | 1.00 | 23.04 |
| 570 | CD2 | LEU | A | 254 | −6.780 | −16.929 | −1.848 | 1.00 | 22.37 |
| 571 | C | LEU | A | 254 | −6.156 | −21.355 | 0.502 | 1.00 | 17.86 |
| 572 | O | LEU | A | 254 | −6.072 | −21.459 | 1.724 | 1.00 | 16.98 |
| 573 | N | SER | A | 255 | −5.357 | −22.043 | −0.300 | 1.00 | 15.32 |
| 574 | CA | SER | A | 255 | −4.340 | −22.876 | 0.308 | 1.00 | 16.43 |
| 575 | CB | SER | A | 255 | −3.810 | −23.881 | −0.676 | 1.00 | 16.76 |
| 576 | OG | SER | A | 255 | −3.416 | −23.202 | −1.825 | 1.00 | 17.59 |
| 577 | C | SER | A | 255 | −3.199 | −21.973 | 0.775 | 1.00 | 16.89 |
| 578 | O | SER | A | 255 | −2.991 | −20.862 | 0.234 | 1.00 | 15.37 |
| 579 | N | ILE | A | 256 | −2.452 | −22.465 | 1.760 | 1.00 | 16.17 |
| 580 | CA | ILE | A | 256 | −1.344 | −21.714 | 2.333 | 1.00 | 18.47 |
| 581 | CB | ILE | A | 256 | −0.580 | −22.511 | 3.457 | 1.00 | 17.13 |
| 582 | CG2 | ILE | A | 256 | 0.288 | −23.648 | 2.868 | 1.00 | 18.77 |
| 583 | CG1 | ILE | A | 256 | 0.333 | −21.575 | 4.219 | 1.00 | 16.65 |
| 584 | CD1 | ILE | A | 256 | −0.402 | −20.528 | 4.973 | 1.00 | 19.69 |
| 585 | C | ILE | A | 256 | −0.406 | −21.258 | 1.228 | 1.00 | 18.89 |
| 586 | O | ILE | A | 256 | 0.061 | −20.115 | 1.243 | 1.00 | 19.36 |
| 587 | N | ALA | A | 257 | −0.169 | −22.122 | 0.244 | 1.00 | 18.48 |
| 588 | CA | ALA | A | 257 | 0.698 | −21.762 | −0.874 | 1.00 | 18.14 |
| 589 | CB | ALA | A | 257 | 0.736 | −22.868 | −1.916 | 1.00 | 15.57 |
| 590 | C | ALA | A | 257 | 0.174 | −20.487 | −1.504 | 1.00 | 18.90 |
| 591 | O | ALA | A | 257 | 0.892 | −19.506 | −1.633 | 1.00 | 20.07 |
| 592 | N | ASP | A | 258 | −1.109 | −20.486 | −1.827 | 1.00 | 19.03 |
| 593 | CA | ASP | A | 258 | −1.721 | −19.346 | −2.483 | 1.00 | 18.65 |
| 594 | CB | ASP | A | 258 | −3.141 | −19.680 | −2.955 | 1.00 | 20.22 |
| 595 | CG | ASP | A | 258 | −3.160 | −20.499 | −4.235 | 1.00 | 21.11 |
| 596 | OD1 | ASP | A | 258 | −4.277 | −20.872 | −4.651 | 1.00 | 23.18 |
| 597 | OD2 | ASP | A | 258 | −2.089 | −20.766 | −4.825 | 1.00 | 19.47 |
| 598 | C | ASP | A | 258 | −1.731 | −18.081 | −1.645 | 1.00 | 17.57 |
| 599 | O | ASP | A | 258 | −1.435 | −17.016 | −2.162 | 1.00 | 16.61 |
| 600 | N | GLN | A | 259 | −2.078 | −18.195 | −0.367 | 1.00 | 16.44 |
| 601 | CA | GLN | A | 259 | −2.121 | −17.036 | 0.510 | 1.00 | 14.15 |
| 602 | CB | GLN | A | 259 | −2.477 | −17.468 | 1.931 | 1.00 | 15.92 |
| 603 | CG | GLN | A | 259 | −3.879 | −18.041 | 2.083 | 1.00 | 15.51 |
| 604 | CD | GLN | A | 259 | −4.215 | −18.391 | 3.511 | 1.00 | 16.36 |
| 605 | OE1 | GLN | A | 259 | −3.718 | −17.788 | 4.433 | 1.00 | 19.80 |
| 606 | NE2 | GLN | A | 259 | −5.080 | −19.358 | 3.694 | 1.00 | 16.26 |
| 607 | C | GLN | A | 259 | −0.768 | −16.325 | 0.487 | 1.00 | 14.18 |
| 608 | O | GLN | A | 259 | −0.714 | −15.098 | 0.373 | 1.00 | 14.26 |
| 609 | N | ILE | A | 260 | 0.314 | −17.107 | 0.523 | 1.00 | 13.39 |
| 610 | CA | ILE | A | 260 | 1.675 | −16.582 | 0.509 | 1.00 | 13.11 |
| 611 | CB | ILE | A | 260 | 2.709 | −17.657 | 0.974 | 1.00 | 11.65 |
| 612 | CG2 | ILE | A | 260 | 4.122 | −17.178 | 0.719 | 1.00 | 12.53 |
| 613 | CG1 | ILE | A | 260 | 2.557 | −17.892 | 2.498 | 1.00 | 13.30 |
| 614 | CD1 | ILE | A | 260 | 3.082 | −19.222 | 3.021 | 1.00 | 12.78 |
| 615 | C | ILE | A | 260 | 2.007 | −15.992 | −0.860 | 1.00 | 14.57 |
| 616 | O | ILE | A | 260 | 2.548 | −14.905 | −0.944 | 1.00 | 14.92 |
| 617 | N | THR | A | 261 | 1.614 | −16.676 | −1.927 | 1.00 | 14.56 |
| 618 | CA | THR | A | 261 | 1.845 | −16.192 | −3.279 | 1.00 | 15.06 |
| 619 | CB | THR | A | 261 | 1.243 | −17.157 | −4.306 | 1.00 | 14.24 |
| 620 | OG1 | THR | A | 261 | 1.938 | −18.405 | −4.240 | 1.00 | 16.34 |
| 621 | CG2 | THR | A | 261 | 1.330 | −16.592 | −5.715 | 1.00 | 15.63 |
| 622 | C | THR | A | 261 | 1.206 | −14.809 | −3.436 | 1.00 | 15.18 |
| 623 | O | THR | A | 261 | 1.865 | −13.862 | −3.848 | 1.00 | 16.32 |
| 624 | N | LEU | A | 262 | −0.065 | −14.679 | −3.085 | 1.00 | 14.08 |
| 625 | CA | LEU | A | 262 | −0.737 | −13.383 | −3.196 | 1.00 | 14.50 |
| 626 | CB | LEU | A | 262 | −2.218 | −13.491 | −2.823 | 1.00 | 12.30 |
| 627 | CG | LEU | A | 262 | −3.094 | −14.247 | −3.828 | 1.00 | 12.27 |
| 628 | CD1 | LEU | A | 262 | −4.510 | −14.299 | −3.279 | 1.00 | 11.50 |
| 629 | CD2 | LEU | A | 262 | −3.043 | −13.549 | −5.170 | 1.00 | 11.07 |
| 630 | C | LEU | A | 262 | −0.095 | −12.305 | −2.333 | 1.00 | 13.40 |
| 631 | O | LEU | A | 262 | 0.019 | −11.144 | −2.759 | 1.00 | 12.89 |
| 632 | N | LEU | A | 263 | 0.270 | −12.672 | −1.113 | 1.00 | 11.86 |
| 633 | CA | LEU | A | 263 | 0.876 | −11.726 | −0.200 | 1.00 | 13.48 |
| 634 | CB | LEU | A | 263 | 1.063 | −12.358 | 1.174 | 1.00 | 13.89 |
| 635 | CG | LEU | A | 263 | −0.173 | −12.363 | 2.061 | 1.00 | 14.62 |
| 636 | CD1 | LEU | A | 263 | 0.037 | −13.259 | 3.256 | 1.00 | 12.39 |
| 637 | CD2 | LEU | A | 263 | −0.477 | −10.934 | 2.485 | 1.00 | 15.87 |
| 638 | C | LEU | A | 263 | 2.206 | −11.248 | −0.746 | 1.00 | 14.62 |
| 639 | O | LEU | A | 263 | 2.486 | −10.053 | −0.771 | 1.00 | 16.35 |
| 640 | N | LYS | A | 264 | 3.026 | −12.168 | −1.225 | 1.00 | 15.83 |
| 641 | CA | LYS | A | 264 | 4.323 | −11.781 | −1.779 | 1.00 | 16.21 |
| 642 | CB | LYS | A | 264 | 5.134 | −13.027 | −2.165 | 1.00 | 16.46 |
| 643 | CG | LYS | A | 264 | 5.888 | −13.666 | −0.985 | 1.00 | 16.55 |
| 644 | CD | LYS | A | 264 | 6.279 | −15.099 | −1.248 | 1.00 | 15.86 |
| 645 | CE | LYS | A | 264 | 7.210 | −15.570 | −0.144 | 1.00 | 17.98 |
| 646 | NZ | LYS | A | 264 | 8.553 | −14.962 | −0.342 | 1.00 | 16.92 |
| 647 | C | LYS | A | 264 | 4.180 | −10.871 | −2.994 | 1.00 | 15.33 |
| 648 | O | LYS | A | 264 | 4.859 | −9.867 | −3.106 | 1.00 | 15.30 |
| 649 | N | ALA | A | 265 | 3.274 | −11.232 | −3.889 | 1.00 | 14.92 |
| 650 | CA | ALA | A | 265 | 3.049 | −10.486 | −5.115 | 1.00 | 16.32 |
| 651 | CB | ALA | A | 265 | 2.241 | −11.340 | −6.070 | 1.00 | 14.20 |
| 652 | C | ALA | A | 265 | 2.418 | −9.092 | −4.995 | 1.00 | 16.04 |
| 653 | O | ALA | A | 265 | 2.645 | −8.236 | −5.850 | 1.00 | 16.12 |
| 654 | N | ALA | A | 266 | 1.644 | −8.851 | −3.942 | 1.00 | 15.90 |
| 655 | CA | ALA | A | 266 | 0.962 | −7.557 | −3.766 | 1.00 | 17.39 |
| 656 | CB | ALA | A | 266 | −0.552 | −7.781 | −3.669 | 1.00 | 16.38 |
| 657 | C | ALA | A | 266 | 1.425 | −6.713 | −2.577 | 1.00 | 17.22 |
| 658 | O | ALA | A | 266 | 1.041 | −5.543 | −2.449 | 1.00 | 16.41 |
| 659 | N | CYS | A | 267 | 2.253 | −7.298 | −1.717 | 1.00 | 15.91 |
| 660 | CA | CYS | A | 267 | 2.710 | −6.602 | −0.527 | 1.00 | 17.69 |
| 661 | CB | CYS | A | 267 | 3.633 | −7.493 | 0.281 | 1.00 | 18.90 |
| 662 | SG | CYS | A | 267 | 3.833 | −6.875 | 1.915 | 1.00 | 23.66 |
| 663 | C | CYS | A | 267 | 3.355 | −5.227 | −0.756 | 1.00 | 16.15 |
| 664 | O | CYS | A | 267 | 2.985 | −4.259 | −0.083 | 1.00 | 14.43 |
| 665 | N | LEU | A | 268 | 4.279 | −5.140 | −1.715 | 1.00 | 14.38 |
| 666 | CA | LEU | A | 268 | 4.923 | −3.873 | −2.018 | 1.00 | 15.09 |
| 667 | CB | LEU | A | 268 | 6.069 | −4.048 | −3.020 | 1.00 | 14.82 |
| 668 | CG | LEU | A | 268 | 7.445 | −3.400 | −2.709 | 1.00 | 16.13 |
| 669 | CD1 | LEU | A | 268 | 8.323 | −3.377 | −3.999 | 1.00 | 13.28 |
| 670 | CD2 | LEU | A | 268 | 7.287 | −1.984 | −2.136 | 1.00 | 13.62 |
| 671 | C | LEU | A | 268 | 3.896 | −2.895 | −2.571 | 1.00 | 15.22 |
| 672 | O | LEU | A | 268 | 3.899 | −1.722 | −2.184 | 1.00 | 15.41 |
| 673 | N | ASP | A | 269 | 3.033 | −3.358 | −3.479 | 1.00 | 14.39 |
| 674 | CA | ASP | A | 269 | 2.001 | −2.494 | −4.043 | 1.00 | 14.73 |
| 675 | CB | ASP | A | 269 | 0.964 | −3.300 | −4.829 | 1.00 | 16.26 |
| 676 | CG | ASP | A | 269 | 1.514 | −3.916 | −6.104 | 1.00 | 16.52 |
| 677 | OD1 | ASP | A | 269 | 0.723 | −4.379 | −6.947 | 1.00 | 19.84 |
| 678 | OD2 | ASP | A | 269 | 2.721 | −3.959 | −6.281 | 1.00 | 18.78 |
| 679 | C | ASP | A | 269 | 1.268 | −1.800 | −2.901 | 1.00 | 16.42 |
| 680 | O | ASP | A | 269 | 1.246 | −0.559 | −2.817 | 1.00 | 17.01 |
| 681 | N | ILE | A | 270 | 0.714 | −2.602 | −1.985 | 1.00 | 16.86 |
| 682 | CA | ILE | A | 270 | −0.061 | −2.066 | −0.854 | 1.00 | 16.75 |
| 683 | CB | ILE | A | 270 | −0.784 | −3.187 | −0.067 | 1.00 | 16.55 |
| 684 | CG2 | ILE | A | 270 | −1.645 | −2.599 | 1.061 | 1.00 | 16.85 |
| 685 | CG1 | ILE | A | 270 | −1.695 | −3.974 | −1.006 | 1.00 | 15.45 |
| 686 | CD1 | ILE | A | 270 | −2.408 | −5.102 | −0.308 | 1.00 | 18.34 |
| 687 | C | ILE | A | 270 | 0.752 | −1.176 | 0.093 | 1.00 | 15.88 |
| 688 | O | ILE | A | 270 | 0.245 | −0.172 | 0.581 | 1.00 | 16.48 |
| 689 | N | LEU | A | 271 | 2.003 | −1.529 | 0.346 | 1.00 | 15.65 |
| 690 | CA | LEU | A | 271 | 2.860 | −0.723 | 1.207 | 1.00 | 16.49 |
| 691 | CB | LEU | A | 271 | 4.223 | −1.405 | 1.302 | 1.00 | 16.24 |
| 692 | CG | LEU | A | 271 | 4.857 | −1.820 | 2.632 | 1.00 | 17.43 |
| 693 | CD1 | LEU | A | 271 | 3.865 | −2.078 | 3.723 | 1.00 | 14.58 |
| 694 | CD2 | LEU | A | 271 | 5.743 | −3.017 | 2.389 | 1.00 | 16.24 |
| 695 | C | LEU | A | 271 | 2.967 | 0.661 | 0.538 | 1.00 | 16.41 |
| 696 | O | LEU | A | 271 | 2.871 | 1.690 | 1.210 | 1.00 | 17.42 |
| 697 | N | MET | A | 272 | 3.073 | 0.679 | −0.795 | 1.00 | 16.60 |
| 698 | CA | MET | A | 272 | 3.172 | 1.927 | −1.559 | 1.00 | 16.79 |
| 699 | CB | MET | A | 272 | 3.575 | 1.666 | −3.020 | 1.00 | 17.65 |
| 700 | CG | MET | A | 272 | 4.996 | 1.199 | −3.221 | 1.00 | 18.79 |
| 701 | SD | MET | A | 272 | 6.223 | 2.515 | −3.063 | 1.00 | 22.97 |
| 702 | CE | MET | A | 272 | 7.580 | 1.837 | −4.003 | 1.00 | 19.64 |
| 703 | C | MET | A | 272 | 1.846 | 2.702 | −1.524 | 1.00 | 17.44 |
| 704 | O | MET | A | 272 | 1.824 | 3.940 | −1.380 | 1.00 | 19.90 |
| 705 | N | LEU | A | 273 | 0.733 | 2.007 | −1.683 | 1.00 | 14.90 |
| 706 | CA | LEU | A | 273 | −0.530 | 2.703 | −1.632 | 1.00 | 14.87 |
| 707 | CB | LEU | A | 273 | −1.661 | 1.728 | −1.888 | 1.00 | 15.48 |
| 708 | CG | LEU | A | 273 | −3.038 | 2.357 | −1.833 | 1.00 | 16.54 |
| 709 | CD1 | LEU | A | 273 | −3.136 | 3.412 | −2.933 | 1.00 | 17.29 |
| 710 | CD2 | LEU | A | 273 | −4.084 | 1.266 | −2.023 | 1.00 | 15.37 |
| 711 | C | LEU | A | 273 | −0.688 | 3.333 | −0.249 | 1.00 | 15.34 |
| 712 | O | LEU | A | 273 | −1.083 | 4.493 | −0.131 | 1.00 | 15.36 |
| 713 | N | ARG | A | 274 | −0.330 | 2.573 | 0.789 | 1.00 | 15.34 |
| 714 | CA | ARG | A | 274 | −0.442 | 3.028 | 2.164 | 1.00 | 15.78 |
| 715 | CB | ARG | A | 274 | −0.097 | 1.919 | 3.150 | 1.00 | 16.75 |
| 716 | CG | ARG | A | 274 | −1.222 | 0.873 | 3.234 | 1.00 | 17.30 |
| 717 | CD | ARG | A | 274 | −1.044 | −0.105 | 4.365 | 1.00 | 17.86 |
| 718 | NE | ARG | A | 274 | −2.252 | −0.918 | 4.516 | 1.00 | 21.93 |
| 719 | CZ | ARG | A | 274 | −2.520 | −1.701 | 5.561 | 1.00 | 22.23 |
| 720 | NH1 | ARG | A | 274 | −1.663 | −1.788 | 6.576 | 1.00 | 21.99 |
| 721 | NH2 | ARG | A | 274 | −3.657 | −2.384 | 5.597 | 1.00 | 20.01 |
| 722 | C | ARG | A | 274 | 0.321 | 4.301 | 2.459 | 1.00 | 17.06 |
| 723 | O | ARG | A | 274 | −0.306 | 5.279 | 2.816 | 1.00 | 18.66 |
| 724 | N | ILE | A | 275 | 1.645 | 4.349 | 2.279 | 1.00 | 18.51 |
| 725 | CA | ILE | A | 275 | 2.328 | 5.618 | 2.564 | 1.00 | 18.34 |
| 726 | GB | ILE | A | 275 | 3.847 | 5.555 | 2.521 | 1.00 | 18.08 |
| 727 | CG2 | ILE | A | 275 | 4.420 | 5.601 | 3.927 | 1.00 | 19.84 |
| 728 | CG1 | ILE | A | 275 | 4.321 | 4.447 | 1.600 | 1.00 | 18.48 |
| 729 | CD1 | ILE | A | 275 | 4.510 | 4.949 | 0.201 | 1.00 | 17.63 |
| 730 | C | ILE | A | 275 | 1.878 | 6.778 | 1.680 | 1.00 | 18.77 |
| 731 | O | ILE | A | 275 | 1.843 | 7.911 | 2.154 | 1.00 | 18.87 |
| 732 | N | CYS | A | 276 | 1.522 | 6.504 | 0.419 | 1.00 | 17.07 |
| 733 | CA | CYS | A | 276 | 1.058 | 7.554 | −0.488 | 1.00 | 16.63 |
| 734 | CB | CYS | A | 276 | 0.985 | 7.062 | −1.931 | 1.00 | 15.81 |
| 735 | SG | CYS | A | 276 | 2.610 | 6.888 | −2.627 | 1.00 | 22.23 |
| 736 | C | CYS | A | 276 | −0.279 | 8.164 | −0.058 | 1.00 | 15.45 |
| 737 | O | CYS | A | 276 | −0.587 | 9.272 | −0.468 | 1.00 | 15.53 |
| 738 | N | THR | A | 277 | −1.064 | 7.449 | 0.746 | 1.00 | 13.95 |
| 739 | CA | THR | A | 277 | −2.321 | 7.993 | 1.249 | 1.00 | 15.92 |
| 740 | CB | THR | A | 277 | −3.363 | 6.887 | 1.545 | 1.00 | 18.17 |
| 741 | OG1 | THR | A | 277 | −2.890 | 6.051 | 2.601 | 1.00 | 20.50 |
| 742 | CG2 | THR | A | 277 | −3.591 | 6.023 | 0.314 | 1.00 | 19.00 |
| 743 | C | THR | A | 277 | −2.054 | 8.816 | 2.520 | 1.00 | 17.31 |
| 744 | O | THR | A | 277 | −2.917 | 9.560 | 2.984 | 1.00 | 19.20 |
| 745 | N | ARG | A | 278 | −0.842 | 8.691 | 3.060 | 1.00 | 17.51 |
| 746 | CA | ARG | A | 278 | −0.386 | 9.411 | 4.266 | 1.00 | 17.48 |
| 747 | CB | ARG | A | 278 | 0.459 | 8.455 | 5.101 | 1.00 | 17.18 |
| 748 | CG | ARG | A | 278 | −0.336 | 7.370 | 5.783 | 1.00 | 16.64 |
| 749 | CD | ARG | A | 278 | −0.683 | 7.825 | 7.175 | 1.00 | 17.73 |
| 750 | NE | ARG | A | 278 | −1.172 | 6.755 | 8.034 | 1.00 | 17.21 |
| 751 | CZ | ARG | A | 278 | −0.487 | 5.667 | 8.346 | 1.00 | 19.13 |
| 752 | NH1 | ARG | A | 278 | 0.733 | 5.484 | 7.861 | 1.00 | 20.62 |
| 753 | NH2 | ARG | A | 278 | −1.017 | 4.773 | 9.171 | 1.00 | 20.16 |
| 754 | C | ARG | A | 278 | 0.459 | 10.667 | 3.891 | 1.00 | 16.92 |
| 755 | O | ARG | A | 278 | 1.327 | 11.130 | 4.637 | 1.00 | 16.64 |
| 756 | N | TYR | A | 279 | 0.173 | 11.226 | 2.727 | 1.00 | 17.14 |
| 757 | CA | TYR | A | 279 | 0.910 | 12.362 | 2.248 | 1.00 | 15.32 |
| 758 | CB | TYR | A | 279 | 1.208 | 12.142 | 0.784 | 1.00 | 14.96 |
| 759 | CG | TYR | A | 279 | 1.709 | 13.344 | 0.060 | 1.00 | 15.39 |
| 760 | CD1 | TYR | A | 279 | 2.998 | 13.831 | 0.265 | 1.00 | 15.10 |
| 761 | CE1 | TYR | A | 279 | 3.451 | 14.915 | −0.457 | 1.00 | 15.87 |
| 762 | CD2 | TYR | A | 279 | 0.898 | 13.977 | −0.875 | 1.00 | 16.01 |
| 763 | CE2 | TYR | A | 279 | 1.341 | 15.050 | −1.591 | 1.00 | 18.57 |
| 764 | CZ | TYR | A | 279 | 2.607 | 15.513 | −1.388 | 1.00 | 17.47 |
| 765 | OH | TYR | A | 279 | 3.019 | 16.564 | −2.161 | 1.00 | 20.53 |
| 766 | C | TYR | A | 279 | 0.192 | 13.667 | 2.493 | 1.00 | 15.96 |
| 767 | O | TYR | A | 279 | −1.030 | 13.751 | 2.351 | 1.00 | 15.17 |
| 768 | N | THR | A | 280 | 0.965 | 14.663 | 2.935 | 1.00 | 17.68 |
| 769 | CA | THR | A | 280 | 0.468 | 16.014 | 3.218 | 1.00 | 18.60 |
| 770 | CB | THR | A | 280 | 0.888 | 16.507 | 4.600 | 1.00 | 18.35 |
| 771 | OG1 | THR | A | 280 | 0.494 | 15.553 | 5.593 | 1.00 | 19.23 |
| 772 | CG2 | THR | A | 280 | 0.196 | 17.793 | 4.905 | 1.00 | 19.36 |
| 773 | C | THR | A | 280 | 1.085 | 16.927 | 2.195 | 1.00 | 18.63 |
| 774 | O | THR | A | 280 | 2.275 | 17.213 | 2.256 | 1.00 | 19.24 |
| 775 | N | PRO | A | 281 | 0.286 | 17.392 | 1.233 | 1.00 | 19.26 |
| 776 | CD | PRO | A | 281 | −1.165 | 17.253 | 1.086 | 1.00 | 19.67 |
| 777 | CA | PRO | A | 281 | 0.791 | 18.273 | 0.201 | 1.00 | 23.13 |
| 778 | CB | PRO | A | 281 | −0.469 | 18.600 | −0.606 | 1.00 | 21.19 |
| 779 | CG | PRO | A | 281 | −1.326 | 17.477 | −0.375 | 1.00 | 19.03 |
| 780 | C | PRO | A | 281 | 1.395 | 19.528 | 0.825 | 1.00 | 27.05 |
| 781 | O | PRO | A | 281 | 2.521 | 19.877 | 0.507 | 1.00 | 29.83 |
| 782 | N | GLU | A | 282 | 0.678 | 20.154 | 1.765 | 1.00 | 29.52 |
| 783 | CA | GLU | A | 282 | 1.132 | 21.389 | 2.435 | 1.00 | 30.92 |
| 784 | CB | GLU | A | 282 | 0.188 | 21.733 | 3.623 | 1.00 | 34.18 |
| 785 | CG | GLU | A | 282 | −0.569 | 23.095 | 3.507 | 1.00 | 37.63 |
| 786 | CD | GLU | A | 282 | 0.121 | 24.277 | 4.231 | 1.00 | 39.35 |
| 787 | OE1 | GLU | A | 282 | 1.126 | 24.839 | 3.717 | 1.00 | 39.81 |
| 788 | OE2 | GLU | A | 282 | −0.365 | 24.656 | 5.320 | 1.00 | 40.91 |
| 789 | C | GLU | A | 282 | 2.602 | 21.384 | 2.907 | 1.00 | 29.16 |
| 790 | O | GLU | A | 282 | 3.380 | 22.301 | 2.610 | 1.00 | 29.44 |
| 791 | N | GLN | A | 283 | 2.967 | 20.336 | 3.629 | 1.00 | 26.73 |
| 792 | CA | GLN | A | 283 | 4.298 | 20.181 | 4.168 | 1.00 | 25.15 |
| 793 | CB | GLN | A | 283 | 4.178 | 19.501 | 5.509 | 1.00 | 25.77 |
| 794 | CG | GLN | A | 283 | 3.161 | 20.158 | 6.363 | 1.00 | 26.99 |
| 795 | CD | GLN | A | 283 | 3.640 | 21.516 | 6.757 | 1.00 | 29.54 |
| 796 | OE1 | GLN | A | 283 | 4.836 | 21.714 | 7.016 | 1.00 | 30.71 |
| 797 | NE2 | GLN | A | 283 | 2.728 | 22.477 | 6.793 | 1.00 | 31.58 |
| 798 | C | GLN | A | 283 | 5.197 | 19.326 | 3.301 | 1.00 | 25.04 |
| 799 | O | GLN | A | 283 | 6.388 | 19.230 | 3.583 | 1.00 | 25.52 |
| 800 | N | ASP | A | 284 | 4.625 | 18.687 | 2.276 | 1.00 | 23.29 |
| 801 | CA | ASP | A | 284 | 5.365 | 17.786 | 1.399 | 1.00 | 20.93 |
| 802 | CB | ASP | A | 284 | 6.450 | 18.520 | 0.622 | 1.00 | 21.51 |
| 803 | CG | ASP | A | 284 | 6.930 | 17.737 | −0.581 | 1.00 | 20.88 |
| 804 | OD1 | ASP | A | 284 | 6.080 | 17.221 | −1.352 | 1.00 | 18.48 |
| 805 | OD2 | ASP | A | 284 | 8.162 | 17.677 | −0.770 | 1.00 | 20.71 |
| 806 | C | ASP | A | 284 | 5.995 | 16.686 | 2.253 | 1.00 | 19.57 |
| 807 | O | ASP | A | 284 | 7.209 | 16.437 | 2.189 | 1.00 | 17.45 |
| 808 | N | THR | A | 285 | 5.154 | 16.058 | 3.073 | 1.00 | 19.17 |
| 809 | CA | THR | A | 285 | 5.578 | 14.983 | 3.955 | 1.00 | 18.19 |
| 810 | CB | THR | A | 285 | 5.501 | 15.404 | 5.451 | 1.00 | 18.36 |
| 811 | OG1 | THR | A | 285 | 4.152 | 15.773 | 5.781 | 1.00 | 17.95 |
| 812 | CG2 | THR | A | 285 | 6.471 | 16.543 | 5.763 | 1.00 | 16.08 |
| 813 | C | THR | A | 285 | 4.699 | 13.742 | 3.817 | 1.00 | 17.40 |
| 814 | O | THR | A | 285 | 3.649 | 13.779 | 3.173 | 1.00 | 16.90 |
| 815 | N | MET | A | 286 | 5.163 | 12.654 | 4.429 | 1.00 | 17.39 |
| 816 | CA | MET | A | 286 | 4.462 | 11.375 | 4.498 | 1.00 | 17.47 |
| 817 | CB | MET | A | 286 | 5.031 | 10.337 | 3.509 | 1.00 | 16.08 |
| 818 | CG | MET | A | 286 | 4.692 | 10.646 | 2.053 | 1.00 | 15.31 |
| 819 | SD | MET | A | 286 | 5.340 | 9.441 | 0.905 | 1.00 | 21.84 |
| 820 | CE | MET | A | 286 | 4.558 | 9.994 | −0.514 | 1.00 | 13.72 |
| 821 | C | MET | A | 286 | 4.609 | 10.893 | 5.951 | 1.00 | 17.11 |
| 822 | O | MET | A | 286 | 5.672 | 11.040 | 6.579 | 1.00 | 16.22 |
| 823 | N | THR | A | 287 | 3.515 | 10.377 | 6.496 | 1.00 | 15.98 |
| 824 | CA | THR | A | 287 | 3.472 | 9.905 | 7.872 | 1.00 | 15.63 |
| 825 | CB | THR | A | 287 | 2.345 | 10.639 | 8.617 | 1.00 | 14.95 |
| 826 | OG1 | THR | A | 287 | 2.591 | 12.039 | 8.503 | 1.00 | 16.07 |
| 827 | CG2 | THR | A | 287 | 2.291 | 10.260 | 10.098 | 1.00 | 14.81 |
| 828 | C | THR | A | 287 | 3.272 | 8.387 | 7.937 | 1.00 | 15.17 |
| 829 | O | THR | A | 287 | 2.454 | 7.825 | 7.221 | 1.00 | 14.62 |
| 830 | N | PHE | A | 288 | 4.105 | 7.732 | 8.737 | 1.00 | 15.64 |
| 831 | CA | PHE | A | 288 | 4.060 | 6.292 | 8.927 | 1.00 | 16.95 |
| 832 | CB | PHE | A | 288 | 5.463 | 5.770 | 9.263 | 1.00 | 16.87 |
| 833 | CG | PHE | A | 288 | 6.443 | 5.919 | 8.134 | 1.00 | 16.91 |
| 834 | CD1 | PHE | A | 288 | 6.982 | 7.164 | 7.818 | 1.00 | 18.03 |
| 835 | CD2 | PHE | A | 288 | 6.782 | 4.831 | 7.345 | 1.00 | 15.86 |
| 836 | CE1 | PHE | A | 288 | 7.827 | 7.309 | 6.733 | 1.00 | 15.63 |
| 837 | CE2 | PHE | A | 288 | 7.629 | 4.977 | 6.265 | 1.00 | 13.84 |
| 838 | CZ | PHE | A | 288 | 8.148 | 6.206 | 5.958 | 1.00 | 13.79 |
| 839 | C | PHE | A | 288 | 3.076 | 6.021 | 10.060 | 1.00 | 18.22 |
| 840 | O | PHE | A | 288 | 2.613 | 6.965 | 10.705 | 1.00 | 17.92 |
| 841 | N | SER | A | 289 | 2.796 | 4.750 | 10.345 | 1.00 | 18.84 |
| 842 | CA | SER | A | 289 | 1.825 | 4.374 | 11.369 | 1.00 | 18.84 |
| 843 | CB | SER | A | 289 | 1.475 | 2.918 | 11.235 | 1.00 | 18.32 |
| 844 | OG | SER | A | 289 | 0.975 | 2.710 | 9.939 | 1.00 | 20.62 |
| 845 | C | SER | A | 289 | 2.088 | 4.698 | 12.821 | 1.00 | 19.48 |
| 846 | O | SER | A | 289 | 1.194 | 4.553 | 13.642 | 1.00 | 21.03 |
| 847 | N | ASP | A | 290 | 3.308 | 5.090 | 13.167 | 1.00 | 18.56 |
| 848 | CA | ASP | A | 290 | 3.569 | 5.461 | 14.543 | 1.00 | 16.64 |
| 849 | CB | ASP | A | 290 | 4.910 | 4.879 | 15.021 | 1.00 | 19.14 |
| 850 | CG | ASP | A | 290 | 6.116 | 5.604 | 14.462 | 1.00 | 17.90 |
| 851 | OD1 | ASP | A | 290 | 6.064 | 6.131 | 13.343 | 1.00 | 19.78 |
| 852 | OD2 | ASP | A | 290 | 7.133 | 5.622 | 15.159 | 1.00 | 17.99 |
| 853 | C | ASP | A | 290 | 3.487 | 7.005 | 14.710 | 1.00 | 16.67 |
| 854 | O | ASP | A | 290 | 3.504 | 7.532 | 15.829 | 1.00 | 16.11 |
| 855 | N | GLY | A | 291 | 3.308 | 7.707 | 13.596 | 1.00 | 14.39 |
| 856 | CA | GLY | A | 291 | 3.227 | 9.155 | 13.627 | 1.00 | 14.41 |
| 857 | C | GLY | A | 291 | 4.476 | 9.773 | 13.034 | 1.00 | 15.52 |
| 858 | O | GLY | A | 291 | 4.473 | 10.956 | 12.721 | 1.00 | 16.88 |
| 859 | N | LEU | A | 292 | 5.544 | 8.993 | 12.869 | 1.00 | 13.32 |
| 860 | CA | LEU | A | 292 | 6.759 | 9.534 | 12.290 | 1.00 | 14.58 |
| 861 | CB | LEU | A | 292 | 7.840 | 8.447 | 12.123 | 1.00 | 14.51 |
| 862 | CG | LEU | A | 292 | 9.165 | 8.928 | 11.494 | 1.00 | 14.16 |
| 863 | CD1 | LEU | A | 292 | 9.777 | 10.015 | 12.337 | 1.00 | 12.49 |
| 864 | CD2 | LEU | A | 292 | 10.138 | 7.778 | 11.272 | 1.00 | 10.42 |
| 865 | C | LEU | A | 292 | 6.436 | 10.177 | 10.943 | 1.00 | 13.88 |
| 866 | O | LEU | A | 292 | 5.942 | 9.522 | 10.060 | 1.00 | 15.24 |
| 867 | N | THR | A | 293 | 6.676 | 11.479 | 10.837 | 1.00 | 14.29 |
| 868 | CA | THR | A | 293 | 6.427 | 12.275 | 9.644 | 1.00 | 14.37 |
| 869 | CB | THR | A | 293 | 5.621 | 13.504 | 10.053 | 1.00 | 16.10 |
| 870 | OG1 | THR | A | 293 | 4.435 | 13.056 | 10.720 | 1.00 | 16.90 |
| 871 | CG2 | THR | A | 293 | 5.259 | 14.356 | 8.851 | 1.00 | 14.68 |
| 872 | C | THR | A | 293 | 7.759 | 12.736 | 9.062 | 1.00 | 14.38 |
| 873 | O | THR | A | 293 | 8.475 | 13.494 | 9.697 | 1.00 | 14.25 |
| 874 | N | LEU | A | 294 | 8.108 | 12.271 | 7.871 | 1.00 | 14.86 |
| 875 | CA | LEU | A | 294 | 9.369 | 12.656 | 7.246 | 1.00 | 15.36 |
| 876 | CB | LEU | A | 294 | 10.135 | 11.418 | 6.803 | 1.00 | 11.39 |
| 877 | CG | LEU | A | 294 | 10.406 | 10.358 | 7.871 | 1.00 | 14.10 |
| 878 | CD1 | LEU | A | 294 | 11.173 | 9.176 | 7.269 | 1.00 | 12.89 |
| 879 | CD2 | LEU | A | 294 | 11.209 | 10.954 | 9.008 | 1.00 | 10.35 |
| 880 | C | LEU | A | 294 | 9.121 | 13.518 | 6.027 | 1.00 | 16.14 |
| 881 | O | LEU | A | 294 | 8.156 | 13.308 | 5.307 | 1.00 | 17.38 |
| 882 | N | ASN | A | 295 | 9.955 | 14.521 | 5.803 | 1.00 | 17.13 |
| 883 | CA | ASN | A | 295 | 9.781 | 15.340 | 4.604 | 1.00 | 17.00 |
| 884 | CB | ASN | A | 295 | 10.402 | 16.732 | 4.756 | 1.00 | 18.00 |
| 885 | CG | ASN | A | 295 | 11.907 | 16.705 | 4.955 | 1.00 | 18.40 |
| 886 | OD1 | ASN | A | 295 | 12.585 | 15.744 | 4.622 | 1.00 | 19.72 |
| 887 | ND2 | ASN | A | 295 | 12.435 | 17.787 | 5.494 | 1.00 | 21.69 |
| 888 | C | ASN | A | 295 | 10.362 | 14.588 | 3.412 | 1.00 | 15.70 |
| 889 | O | ASN | A | 295 | 10.872 | 13.477 | 3.573 | 1.00 | 16.12 |
| 890 | N | ARG | A | 296 | 10.265 | 15.163 | 2.217 | 1.00 | 15.71 |
| 891 | CA | ARG | A | 296 | 10.763 | 14.512 | 0.996 | 1.00 | 13.87 |
| 892 | CB | ARG | A | 296 | 10.486 | 15.382 | −0.211 | 1.00 | 14.02 |
| 893 | CG | ARG | A | 296 | 10.843 | 14.721 | −1.480 | 1.00 | 12.59 |
| 894 | CD | ARG | A | 296 | 10.551 | 15.621 | −2.635 | 1.00 | 14.33 |
| 895 | NE | ARG | A | 296 | 9.137 | 15.980 | −2.756 | 1.00 | 14.74 |
| 896 | CZ | ARG | A | 296 | 8.449 | 15.881 | −3.887 | 1.00 | 13.62 |
| 897 | NH1 | ARG | A | 296 | 9.029 | 15.416 | −4.988 | 1.00 | 12.42 |
| 898 | NH2 | ARG | A | 296 | 7.184 | 16.276 | −3.919 | 1.00 | 13.67 |
| 899 | C | ARG | A | 296 | 12.229 | 14.152 | 1.018 | 1.00 | 15.32 |
| 900 | O | ARG | A | 296 | 12.639 | 13.078 | 0.533 | 1.00 | 16.68 |
| 901 | N | THR | A | 297 | 13.037 | 15.046 | 1.576 | 1.00 | 14.40 |
| 902 | CA | THR | A | 297 | 14.455 | 14.806 | 1.675 | 1.00 | 13.98 |
| 903 | CB | THR | A | 297 | 15.152 | 16.025 | 2.275 | 1.00 | 14.79 |
| 904 | OG1 | THR | A | 297 | 15.182 | 17.055 | 1.291 | 1.00 | 16.57 |
| 905 | CG2 | THR | A | 297 | 16.551 | 15.700 | 2.730 | 1.00 | 14.07 |
| 906 | C | THR | A | 297 | 14.710 | 13.581 | 2.527 | 1.00 | 14.36 |
| 907 | O | THR | A | 297 | 15.529 | 12.734 | 2.180 | 1.00 | 14.79 |
| 908 | N | GLN | A | 298 | 14.033 | 13.509 | 3.666 | 1.00 | 15.33 |
| 909 | CA | GLN | A | 298 | 14.176 | 12.383 | 4.576 | 1.00 | 15.06 |
| 910 | CB | GLN | A | 298 | 13.520 | 12.704 | 5.927 | 1.00 | 16.77 |
| 911 | CG | GLN | A | 298 | 14.257 | 13.756 | 6.743 | 1.00 | 15.29 |
| 912 | CD | GLN | A | 298 | 13.446 | 14.272 | 7.923 | 1.00 | 17.60 |
| 913 | OE1 | GLN | A | 298 | 12.222 | 14.328 | 7.873 | 1.00 | 17.10 |
| 914 | NE2 | GLN | A | 298 | 14.132 | 14.713 | 8.964 | 1.00 | 17.23 |
| 915 | C | GLN | A | 298 | 13.657 | 11.054 | 3.973 | 1.00 | 15.13 |
| 916 | O | GLN | A | 298 | 14.256 | 9.999 | 4.218 | 1.00 | 15.27 |
| 917 | N | MET | A | 299 | 12.561 | 11.082 | 3.201 | 1.00 | 15.38 |
| 918 | CA | MET | A | 299 | 12.076 | 9.848 | 2.564 | 1.00 | 15.55 |
| 919 | CB | MET | A | 299 | 10.863 | 10.093 | 1.703 | 1.00 | 12.60 |
| 920 | CG | MET | A | 299 | 9.663 | 10.434 | 2.476 | 1.00 | 15.70 |
| 921 | SD | MET | A | 299 | 9.234 | 9.124 | 3.549 | 1.00 | 17.19 |
| 922 | CE | MET | A | 299 | 8.349 | 8.002 | 2.467 | 1.00 | 16.80 |
| 923 | C | MET | A | 299 | 13.189 | 9.358 | 1.652 | 1.00 | 16.05 |
| 924 | O | MET | A | 299 | 13.461 | 8.167 | 1.572 | 1.00 | 17.34 |
| 925 | N | HIS | A | 300 | 13.835 | 10.302 | 0.967 | 1.00 | 16.17 |
| 926 | CA | HIS | A | 300 | 14.938 | 9.995 | 0.065 | 1.00 | 16.17 |
| 927 | CB | HIS | A | 300 | 15.429 | 11.282 | −0.636 | 1.00 | 15.59 |
| 928 | CG | HIS | A | 300 | 16.578 | 11.061 | −1.561 | 1.00 | 15.33 |
| 929 | CD2 | HIS | A | 300 | 16.620 | 10.844 | −2.897 | 1.00 | 17.23 |
| 930 | ND1 | HIS | A | 300 | 17.885 | 10.994 | −1.123 | 1.00 | 16.64 |
| 931 | CE1 | HIS | A | 300 | 18.675 | 10.737 | −2.149 | 1.00 | 14.56 |
| 932 | NE2 | HIS | A | 300 | 17.937 | 10.641 | −3.236 | 1.00 | 16.30 |
| 933 | C | HIS | A | 300 | 16.081 | 9.345 | 0.831 | 1.00 | 16.05 |
| 934 | O | HIS | A | 300 | 16.565 | 8.265 | 0.455 | 1.00 | 15.26 |
| 935 | N | ASN | A | 301 | 16.475 | 10.004 | 1.919 | 1.00 | 16.64 |
| 936 | CA | ASN | A | 301 | 17.573 | 9.576 | 2.778 | 1.00 | 16.77 |
| 937 | CB | ASN | A | 301 | 17.895 | 10.658 | 3.823 | 1.00 | 16.75 |
| 938 | CG | ASN | A | 301 | 18.406 | 11.957 | 3.184 | 1.00 | 20.30 |
| 939 | OD1 | ASN | A | 301 | 18.804 | 11.967 | 1.999 | 1.00 | 20.59 |
| 940 | ND2 | ASN | A | 301 | 18.335 | 13.066 | 3.931 | 1.00 | 18.69 |
| 941 | C | ASN | A | 301 | 17.288 | 8.276 | 3.460 | 1.00 | 16.93 |
| 942 | O | ASN | A | 301 | 18.206 | 7.536 | 3.774 | 1.00 | 17.99 |
| 943 | N | ALA | A | 302 | 16.012 | 7.988 | 3.680 | 1.00 | 17.82 |
| 944 | CA | ALA | A | 302 | 15.612 | 6.756 | 4.338 | 1.00 | 16.47 |
| 945 | CB | ALA | A | 302 | 14.205 | 6.884 | 4.842 | 1.00 | 15.77 |
| 946 | C | ALA | A | 302 | 15.760 | 5.503 | 3.469 | 1.00 | 16.21 |
| 947 | O | ALA | A | 302 | 15.642 | 4.398 | 3.961 | 1.00 | 16.49 |
| 948 | N | GLY | A | 303 | 16.016 | 5.665 | 2.180 | 1.00 | 17.15 |
| 949 | CA | GLY | A | 303 | 16.162 | 4.500 | 1.337 | 1.00 | 17.25 |
| 950 | C | GLY | A | 303 | 15.488 | 4.587 | −0.019 | 1.00 | 18.29 |
| 951 | O | GLY | A | 303 | 15.826 | 3.816 | −0.916 | 1.00 | 19.54 |
| 952 | N | PHE | A | 304 | 14.551 | 5.511 | −0.204 | 1.00 | 18.25 |
| 953 | CA | PHE | A | 304 | 13.872 | 5.603 | −1.499 | 1.00 | 18.54 |
| 954 | CB | PHE | A | 304 | 12.661 | 6.534 | −1.435 | 1.00 | 19.40 |
| 955 | CG | PHE | A | 304 | 11.458 | 5.915 | −0.797 | 1.00 | 18.61 |
| 956 | CD1 | PHE | A | 304 | 11.297 | 5.929 | 0.581 | 1.00 | 18.46 |
| 957 | CD2 | PHE | A | 304 | 10.496 | 5.294 | −1.574 | 1.00 | 19.63 |
| 958 | CE1 | PHE | A | 304 | 10.193 | 5.329 | 1.167 | 1.00 | 21.04 |
| 959 | CE2 | PHE | A | 304 | 9.385 | 4.690 | −0.991 | 1.00 | 19.15 |
| 960 | CZ | PHE | A | 304 | 9.233 | 4.707 | 0.373 | 1.00 | 18.69 |
| 961 | C | PHE | A | 304 | 14.799 | 6.009 | −2.631 | 1.00 | 18.46 |
| 962 | O | PHE | A | 304 | 14.502 | 5.764 | −3.795 | 1.00 | 17.98 |
| 963 | N | GLY | A | 305 | 15.905 | 6.656 | −2.288 | 1.00 | 18.96 |
| 964 | CA | GLY | A | 305 | 16.880 | 7.063 | −3.279 | 1.00 | 19.43 |
| 965 | C | GLY | A | 305 | 16.334 | 7.777 | −4.501 | 1.00 | 21.11 |
| 966 | O | GLY | A | 305 | 15.509 | 8.673 | −4.375 | 1.00 | 22.17 |
| 967 | N | PRO | A | 306 | 16.788 | 7.415 | −5.710 | 1.00 | 20.61 |
| 968 | CD | PRO | A | 306 | 17.788 | 6.375 | −6.011 | 1.00 | 19.86 |
| 969 | CA | PRO | A | 306 | 16.313 | 8.058 | −6.938 | 1.00 | 21.15 |
| 970 | CB | PRO | A | 306 | 17.242 | 7.469 | −7.992 | 1.00 | 20.67 |
| 971 | CG | PRO | A | 306 | 17.521 | 6.088 | −7.448 | 1.00 | 21.94 |
| 972 | C | PRO | A | 306 | 14.841 | 7.801 | −7.289 | 1.00 | 21.61 |
| 973 | O | PRO | A | 306 | 14.380 | 8.213 | −8.353 | 1.00 | 23.28 |
| 974 | N | LEU | A | 307 | 14.094 | 7.179 | −6.383 | 1.00 | 20.33 |
| 975 | CA | LEU | A | 307 | 12.704 | 6.867 | −6.635 | 1.00 | 18.74 |
| 976 | CB | LEU | A | 307 | 12.469 | 5.399 | −6.291 | 1.00 | 21.17 |
| 977 | CG | LEU | A | 307 | 11.118 | 4.743 | −6.613 | 1.00 | 22.39 |
| 978 | CD1 | LEU | A | 307 | 10.957 | 4.594 | −8.125 | 1.00 | 23.14 |
| 979 | CD2 | LEU | A | 307 | 11.022 | 3.392 | −5.937 | 1.00 | 20.12 |
| 980 | C | LEU | A | 307 | 11.742 | 7.727 | −5.842 | 1.00 | 18.73 |
| 981 | O | LEU | A | 307 | 10.545 | 7.704 | −6.099 | 1.00 | 18.72 |
| 982 | N | THR | A | 308 | 12.262 | 8.554 | −4.938 | 1.00 | 18.50 |
| 983 | CA | THR | A | 308 | 11.408 | 9.364 | −4.086 | 1.00 | 17.36 |
| 984 | CB | THR | A | 308 | 12.168 | 10.000 | −2.895 | 1.00 | 19.93 |
| 985 | OG1 | THR | A | 308 | 12.308 | 11.404 | −3.075 | 1.00 | 22.32 |
| 986 | CG2 | THR | A | 308 | 13.514 | 9.432 | −2.736 | 1.00 | 17.49 |
| 987 | C | THR | A | 308 | 10.517 | 10.405 | −4.740 | 1.00 | 17.72 |
| 988 | O | THR | A | 308 | 9.407 | 10.655 | −4.278 | 1.00 | 17.73 |
| 989 | N | ASP | A | 309 | 10.993 | 11.056 | −5.786 | 1.00 | 18.34 |
| 990 | CA | ASP | A | 309 | 10.167 | 12.075 | −6.434 | 1.00 | 18.34 |
| 991 | CB | ASP | A | 309 | 11.005 | 12.964 | −7.311 | 1.00 | 19.68 |
| 992 | CG | ASP | A | 309 | 12.048 | 13.663 | −6.536 | 1.00 | 21.80 |
| 993 | OD1 | ASP | A | 309 | 13.247 | 13.519 | −6.891 | 1.00 | 23.93 |
| 994 | OD2 | ASP | A | 309 | 11.655 | 14.322 | −5.541 | 1.00 | 22.59 |
| 995 | C | ASP | A | 309 | 9.028 | 11.512 | −7.223 | 1.00 | 17.07 |
| 996 | O | ASP | A | 309 | 8.056 | 12.211 | −7.439 | 1.00 | 16.35 |
| 997 | N | LEU | A | 310 | 9.221 | 10.299 | −7.745 | 1.00 | 19.09 |
| 998 | CA | LEU | A | 310 | 8.204 | 9.559 | −8.497 | 1.00 | 21.41 |
| 999 | CB | LEU | A | 310 | 8.793 | 8.312 | −9.158 | 1.00 | 23.47 |
| 1000 | CG | LEU | A | 310 | 9.273 | 8.446 | −10.593 | 1.00 | 25.11 |
| 1001 | CD1 | LEU | A | 310 | 9.871 | 7.129 | −11.041 | 1.00 | 25.39 |
| 1002 | CD2 | LEU | A | 310 | 8.099 | 8.832 | −11.480 | 1.00 | 25.87 |
| 1003 | C | LEU | A | 310 | 7.118 | 9.110 | −7.525 | 1.00 | 20.39 |
| 1004 | O | LEU | A | 310 | 5.930 | 9.230 | −7.812 | 1.00 | 21.79 |
| 1005 | N | VAL | A | 311 | 7.537 | 8.603 | −6.367 | 1.00 | 18.78 |
| 1006 | CA | VAL | A | 311 | 6.592 | 8.172 | −5.342 | 1.00 | 16.69 |
| 1007 | CB | VAL | A | 311 | 7.333 | 7.597 | −4.124 | 1.00 | 15.13 |
| 1008 | CG1 | VAL | A | 311 | 6.361 | 7.169 | −3.020 | 1.00 | 15.45 |
| 1009 | CG2 | VAL | A | 311 | 8.190 | 6.454 | −4.575 | 1.00 | 15.80 |
| 1010 | C | VAL | A | 311 | 5.763 | 9.376 | −4.927 | 1.00 | 14.96 |
| 1011 | O | VAL | A | 311 | 4.554 | 9.289 | −4.840 | 1.00 | 14.98 |
| 1012 | N | PHE | A | 312 | 6.422 | 10.515 | −4.720 | 1.00 | 16.10 |
| 1013 | CA | PHE | A | 312 | 5.744 | 11.749 | −4.312 | 1.00 | 15.43 |
| 1014 | CB | PHE | A | 312 | 6.735 | 12.837 | −3.932 | 1.00 | 16.20 |
| 1015 | CG | PHE | A | 312 | 7.194 | 12.758 | −2.504 | 1.00 | 17.26 |
| 1016 | CD1 | PHE | A | 312 | 8.062 | 11.749 | −2.089 | 1.00 | 16.99 |
| 1017 | CD2 | PHE | A | 312 | 6.723 | 13.668 | −1.562 | 1.00 | 13.96 |
| 1018 | CE1 | PHE | A | 312 | 8.442 | 11.652 | −0.762 | 1.00 | 16.48 |
| 1019 | CE2 | PHE | A | 312 | 7.098 | 13.575 | −0.247 | 1.00 | 13.39 |
| 1020 | CZ | PHE | A | 312 | 7.959 | 12.566 | 0.157 | 1.00 | 15.65 |
| 1021 | C | PHE | A | 312 | 4.836 | 12.257 | −5.385 | 1.00 | 15.11 |
| 1022 | O | PHE | A | 312 | 3.819 | 12.847 | −5.092 | 1.00 | 16.14 |
| 1023 | N | ALA | A | 313 | 5.207 | 12.031 | −6.633 | 1.00 | 15.74 |
| 1024 | CA | ALA | A | 313 | 4.384 | 12.462 | −7.745 | 1.00 | 16.86 |
| 1025 | CB | ALA | A | 313 | 5.200 | 12.487 | −9.027 | 1.00 | 18.61 |
| 1026 | C | ALA | A | 313 | 3.174 | 11.534 | −7.873 | 1.00 | 17.47 |
| 1027 | O | ALA | A | 313 | 2.081 | 11.989 | −8.221 | 1.00 | 17.51 |
| 1028 | N | PHE | A | 314 | 3.363 | 10.243 | −7.592 | 1.00 | 16.65 |
| 1029 | CA | PHE | A | 314 | 2.266 | 9.264 | −7.638 | 1.00 | 16.43 |
| 1030 | CB | PHE | A | 314 | 2.783 | 7.831 | −7.446 | 1.00 | 16.00 |
| 1031 | CG | PHE | A | 314 | 1.683 | 6.812 | −7.234 | 1.00 | 19.00 |
| 1032 | CD1 | PHE | A | 314 | 1.002 | 6.270 | −8.316 | 1.00 | 19.62 |
| 1033 | CD2 | PHE | A | 314 | 1.262 | 6.460 | −5.936 | 1.00 | 20.46 |
| 1034 | CE1 | PHE | A | 314 | −0.090 | 5.401 | −8.111 | 1.00 | 20.93 |
| 1035 | CE2 | PHE | A | 314 | 0.167 | 5.590 | −5.724 | 1.00 | 21.50 |
| 1036 | CZ | PHE | A | 314 | −0.510 | 5.067 | −6.813 | 1.00 | 20.23 |
| 1037 | C | PHE | A | 314 | 1.284 | 9.602 | −6.520 | 1.00 | 15.99 |
| 1038 | O | PHE | A | 314 | 0.063 | 9.549 | −6.707 | 1.00 | 15.93 |
| 1039 | N | ALA | A | 315 | 1.827 | 9.928 | −5.348 | 1.00 | 16.04 |
| 1040 | CA | ALA | A | 315 | 1.013 | 10.310 | −4.198 | 1.00 | 16.36 |
| 1041 | CB | ALA | A | 315 | 1.887 | 10.566 | −2.991 | 1.00 | 15.32 |
| 1042 | C | ALA | A | 315 | 0.211 | 11.555 | −4.554 | 1.00 | 17.37 |
| 1043 | O | ALA | A | 315 | −0.991 | 11.588 | −4.352 | 1.00 | 17.22 |
| 1044 | N | GLY | A | 316 | 0.869 | 12.556 | −5.135 | 1.00 | 17.35 |
| 1045 | CA | GLY | A | 316 | 0.170 | 13.772 | −5.518 | 1.00 | 18.94 |
| 1046 | C | GLY | A | 316 | −0.985 | 13.482 | −6.461 | 1.00 | 19.73 |
| 1047 | O | GLY | A | 316 | −2.080 | 14.009 | −6.288 | 1.00 | 20.12 |
| 1048 | N | GLN | A | 317 | −0.762 | 12.589 | −7.420 | 1.00 | 20.89 |
| 1049 | CA | GLN | A | 317 | −1.790 | 12.235 | −8.387 | 1.00 | 21.78 |
| 1050 | CB | GLN | A | 317 | −1.219 | 11.444 | −9.571 | 1.00 | 24.33 |
| 1051 | CG | GLN | A | 317 | 0.083 | 11.942 | −10.165 | 1.00 | 29.59 |
| 1052 | CD | GLN | A | 317 | 0.028 | 13.378 | −10.618 | 1.00 | 31.66 |
| 1053 | OE1 | GLN | A | 317 | −0.293 | 13.660 | −11.777 | 1.00 | 36.70 |
| 1054 | NE2 | GLN | A | 317 | 0.406 | 14.296 | −9.735 | 1.00 | 32.00 |
| 1055 | C | GLN | A | 317 | −2.898 | 11.416 | −7.753 | 1.00 | 22.05 |
| 1056 | O | GLN | A | 317 | −3.886 | 11.132 | −8.413 | 1.00 | 23.27 |
| 1057 | N | LEU | A | 318 | −2.715 | 10.972 | −6.514 | 1.00 | 20.74 |
| 1058 | CA | LEU | A | 318 | −3.752 | 10.203 | −5.826 | 1.00 | 21.03 |
| 1059 | CB | LEU | A | 318 | −3.123 | 9.373 | −4.709 | 1.00 | 22.39 |
| 1060 | CG | LEU | A | 318 | −3.523 | 7.928 | −4.446 | 1.00 | 23.30 |
| 1061 | CD1 | LEU | A | 318 | −3.281 | 7.089 | −5.691 | 1.00 | 24.32 |
| 1062 | CD2 | LEU | A | 318 | −2.729 | 7.374 | −3.269 | 1.00 | 23.64 |
| 1063 | C | LEU | A | 318 | −4.742 | 11.190 | −5.207 | 1.00 | 21.50 |
| 1064 | O | LEU | A | 318 | −5.926 | 10.916 | −5.102 | 1.00 | 21.19 |
| 1065 | N | LEU | A | 319 | −4.248 | 12.365 | −4.830 | 1.00 | 22.63 |
| 1066 | CA | LEU | A | 319 | −5.070 | 13.400 | −4.204 | 1.00 | 23.25 |
| 1067 | CB | LEU | A | 319 | −4.273 | 14.686 | −4.051 | 1.00 | 23.72 |
| 1068 | CG | LEU | A | 319 | −3.003 | 14.592 | −3.232 | 1.00 | 25.21 |
| 1069 | CD1 | LEU | A | 319 | −2.286 | 15.940 | −3.333 | 1.00 | 27.97 |
| 1070 | CD2 | LEU | A | 319 | −3.328 | 14.206 | −1.777 | 1.00 | 25.70 |
| 1071 | C | LEU | A | 319 | −6.410 | 13.706 | −4.873 | 1.00 | 23.85 |
| 1072 | O | LEU | A | 319 | −7.439 | 13.673 | −4.205 | 1.00 | 25.23 |
| 1073 | N | PRO | A | 320 | −6.409 | 14.089 | −6.174 | 1.00 | 24.07 |
| 1074 | CD | PRO | A | 320 | −5.220 | 14.333 | −7.024 | 1.00 | 24.33 |
| 1075 | CA | PRO | A | 320 | −7.639 | 14.399 | −6.918 | 1.00 | 23.26 |
| 1076 | CB | PRO | A | 320 | −7.147 | 14.461 | −8.359 | 1.00 | 25.29 |
| 1077 | CG | PRO | A | 320 | −5.792 | 15.070 | −8.216 | 1.00 | 24.51 |
| 1078 | C | PRO | A | 320 | −8.706 | 13.318 | −6.785 | 1.00 | 22.41 |
| 1079 | O | PRO | A | 320 | −9.890 | 13.602 | −6.957 | 1.00 | 22.31 |
| 1080 | N | LEU | A | 321 | −8.282 | 12.074 | −6.542 | 1.00 | 20.32 |
| 1081 | CA | LEU | A | 321 | −9.218 | 10.976 | −6.380 | 1.00 | 18.93 |
| 1082 | CB | LEU | A | 321 | −8.507 | 9.622 | −6.432 | 1.00 | 18.78 |
| 1083 | CG | LEU | A | 321 | −8.308 | 9.035 | −7.843 | 1.00 | 18.00 |
| 1084 | CD1 | LEU | A | 321 | −7.322 | 9.867 | −8.584 | 1.00 | 20.63 |
| 1085 | CD2 | LEU | A | 321 | −7.843 | 7.596 | −7.791 | 1.00 | 17.88 |
| 1086 | C | LEU | A | 321 | −10.043 | 11.082 | −5.108 | 1.00 | 19.17 |
| 1087 | O | LEU | A | 321 | −11.062 | 10.410 | −4.981 | 1.00 | 20.44 |
| 1088 | N | GLU | A | 322 | −9.577 | 11.866 | −4.143 | 1.00 | 19.27 |
| 1089 | CA | GLU | A | 322 | −10.290 | 12.061 | −2.867 | 1.00 | 20.00 |
| 1090 | CB | GLU | A | 322 | −11.350 | 13.156 | −3.000 | 1.00 | 20.61 |
| 1091 | CG | GLU | A | 322 | −10.827 | 14.420 | −3.641 | 1.00 | 25.44 |
| 1092 | CD | GLU | A | 322 | −11.576 | 15.666 | −3.219 | 1.00 | 28.32 |
| 1093 | OE1 | GLU | A | 322 | −12.037 | 15.749 | −2.053 | 1.00 | 30.30 |
| 1094 | OE2 | GLU | A | 322 | −11.670 | 16.588 | −4.051 | 1.00 | 30.01 |
| 1095 | C | GLU | A | 322 | −10.954 | 10.800 | −2.304 | 1.00 | 19.37 |
| 1096 | O | GLU | A | 322 | −12.119 | 10.820 | −1.932 | 1.00 | 20.57 |
| 1097 | N | MET | A | 323 | −10.200 | 9.713 | −2.248 | 1.00 | 18.99 |
| 1098 | CA | MET | A | 323 | −10.670 | 8.441 | −1.736 | 1.00 | 17.81 |
| 1099 | CB | MET | A | 323 | −9.713 | 7.322 | −2.174 | 1.00 | 19.71 |
| 1100 | CG | MET | A | 323 | −9.790 | 6.904 | −3.653 | 1.00 | 21.26 |
| 1101 | SD | MET | A | 323 | −8.392 | 5.832 | −4.116 | 1.00 | 22.21 |
| 1102 | CE | MET | A | 323 | −8.757 | 4.374 | −3.384 | 1.00 | 22.92 |
| 1103 | C | MET | A | 323 | −10.743 | 8.430 | −0.212 | 1.00 | 17.71 |
| 1104 | O | MET | A | 323 | −9.899 | 9.034 | 0.475 | 1.00 | 19.02 |
| 1105 | N | ASP | A | 324 | −11.721 | 7.710 | 0.336 | 1.00 | 16.79 |
| 1106 | CA | ASP | A | 324 | −11.809 | 7.612 | 1.792 | 1.00 | 15.53 |
| 1107 | CB | ASP | A | 324 | −13.250 | 7.787 | 2.304 | 1.00 | 15.59 |
| 1108 | CG | ASP | A | 324 | −14.227 | 6.832 | 1.676 | 1.00 | 15.20 |
| 1109 | OD1 | ASP | A | 324 | −13.848 | 6.064 | 0.789 | 1.00 | 16.66 |
| 1110 | OD2 | ASP | A | 324 | −15.385 | 6.860 | 2.089 | 1.00 | 15.60 |
| 1111 | C | ASP | A | 324 | −11.171 | 6.317 | 2.248 | 1.00 | 14.20 |
| 1112 | O | ASP | A | 324 | −10.575 | 5.616 | 1.445 | 1.00 | 14.53 |
| 1113 | N | ASP | A | 325 | −11.242 | 6.014 | 3.532 | 1.00 | 15.62 |
| 1114 | CA | ASP | A | 325 | −10.639 | 4.791 | 4.059 | 1.00 | 16.98 |
| 1115 | CB | ASP | A | 325 | −10.704 | 4.758 | 5.597 | 1.00 | 22.06 |
| 1116 | CG | ASP | A | 325 | −9.869 | 5.869 | 6.267 | 1.00 | 27.41 |
| 1117 | OD1 | ASP | A | 325 | −8.947 | 6.436 | 5.627 | 1.00 | 28.96 |
| 1118 | OD2 | ASP | A | 325 | −10.141 | 6.172 | 7.464 | 1.00 | 31.89 |
| 1119 | C | ASP | A | 325 | −11.263 | 3.503 | 3.494 | 1.00 | 16.36 |
| 1120 | O | ASP | A | 325 | −10.576 | 2.507 | 3.290 | 1.00 | 16.22 |
| 1121 | N | THR | A | 326 | −12.565 | 3.495 | 3.252 | 1.00 | 17.30 |
| 1122 | CA | THR | A | 326 | −13.161 | 2.283 | 2.722 | 1.00 | 15.19 |
| 1123 | CB | THR | A | 326 | −14.687 | 2.297 | 2.832 | 1.00 | 16.45 |
| 1124 | OG1 | THR | A | 326 | −15.235 | 3.016 | 1.729 | 1.00 | 23.49 |
| 1125 | CG2 | THR | A | 326 | −15.134 | 2.966 | 4.100 | 1.00 | 13.11 |
| 1126 | C | THR | A | 326 | −12.695 | 2.044 | 1.282 | 1.00 | 13.91 |
| 1127 | O | THR | A | 326 | −12.304 | 0.945 | 0.956 | 1.00 | 14.30 |
| 1128 | N | GLU | A | 327 | −12.669 | 3.087 | 0.451 | 1.00 | 13.54 |
| 1129 | CA | GLU | A | 327 | −12.217 | 2.993 | −0.950 | 1.00 | 14.50 |
| 1130 | CB | GLU | A | 327 | −12.342 | 4.337 | −1.669 | 1.00 | 13.69 |
| 1131 | CG | GLU | A | 327 | −13.739 | 4.596 | −2.188 | 1.00 | 15.66 |
| 1132 | CD | GLU | A | 327 | −14.061 | 6.070 | −2.461 | 1.00 | 15.47 |
| 1133 | OE1 | GLU | A | 327 | −15.220 | 6.353 | −2.821 | 1.00 | 15.80 |
| 1134 | OE2 | GLU | A | 327 | −13.187 | 6.940 | −2.322 | 1.00 | 14.96 |
| 1135 | C | GLU | A | 327 | −10.779 | 2.530 | −1.005 | 1.00 | 13.39 |
| 1136 | O | GLU | A | 327 | −10.410 | 1.699 | −1.835 | 1.00 | 13.39 |
| 1137 | N | THR | A | 328 | −9.962 | 3.095 | −0.132 | 1.00 | 12.39 |
| 1138 | CA | THR | A | 328 | −8.561 | 2.694 | −0.046 | 1.00 | 14.32 |
| 1139 | CB | THR | A | 328 | −7.788 | 3.598 | 0.931 | 1.00 | 12.84 |
| 1140 | OG1 | THR | A | 328 | −7.866 | 4.957 | 0.491 | 1.00 | 14.19 |
| 1141 | CG2 | THR | A | 328 | −6.377 | 3.177 | 1.007 | 1.00 | 12.73 |
| 1142 | C | THR | A | 328 | −8.489 | 1.229 | 0.434 | 1.00 | 13.38 |
| 1143 | O | THR | A | 328 | −7.683 | 0.436 | −0.065 | 1.00 | 12.40 |
| 1144 | N | GLY | A | 329 | −9.342 | 0.878 | 1.398 | 1.00 | 14.14 |
| 1145 | CA | GLY | A | 329 | −9.367 | −0.473 | 1.917 | 1.00 | 10.87 |
| 1146 | C | GLY | A | 329 | −9.775 | −1.407 | 0.806 | 1.00 | 11.08 |
| 1147 | O | GLY | A | 329 | −9.111 | −2.414 | 0.588 | 1.00 | 11.93 |
| 1148 | N | LEU | A | 330 | −10.815 | −1.037 | 0.061 | 1.00 | 11.42 |
| 1149 | CA | LEU | A | 330 | −11.325 | −1.851 | −1.059 | 1.00 | 13.84 |
| 1150 | CB | LEU | A | 330 | −12.663 | −1.303 | −1.587 | 1.00 | 11.99 |
| 1151 | CG | LEU | A | 330 | −13.901 | −1.664 | −0.741 | 1.00 | 14.67 |
| 1152 | CD1 | LEU | A | 330 | −15.063 | −0.697 | −0.978 | 1.00 | 13.20 |
| 1153 | CD2 | LEU | A | 330 | −14.309 | −3.124 | −1.000 | 1.00 | 13.43 |
| 1154 | C | LEU | A | 330 | −10.314 | −1.973 | −2.202 | 1.00 | 14.49 |
| 1155 | O | LEU | A | 330 | −10.076 | −3.078 | −2.719 | 1.00 | 14.89 |
| 1156 | N | LEU | A | 331 | −9.674 | −0.855 | −2.540 | 1.00 | 13.76 |
| 1157 | CA | LEU | A | 331 | −8.664 | −0.830 | −3.586 | 1.00 | 13.42 |
| 1158 | CB | LEU | A | 331 | −8.134 | 0.600 | −3.768 | 1.00 | 14.33 |
| 1159 | CG | LEU | A | 331 | −7.713 | 1.059 | −5.173 | 1.00 | 16.95 |
| 1160 | CD1 | LEU | A | 331 | −6.203 | 1.142 | −5.329 | 1.00 | 19.20 |
| 1161 | CD2 | LEU | A | 331 | −8.335 | 0.175 | −6.231 | 1.00 | 17.35 |
| 1162 | C | LEU | A | 331 | −7.531 | −1.778 | −3.192 | 1.00 | 12.94 |
| 1163 | O | LEU | A | 331 | −7.024 | −2.520 | −4.021 | 1.00 | 12.82 |
| 1164 | N | SER | A | 332 | −7.150 | −1.775 | −1.919 | 1.00 | 12.43 |
| 1165 | CA | SER | A | 332 | −6.095 | −2.686 | −1.448 | 1.00 | 12.96 |
| 1166 | CB | SER | A | 332 | −5.725 | −2.391 | 0.004 | 1.00 | 12.28 |
| 1167 | OG | SER | A | 332 | −5.214 | −1.089 | 0.125 | 1.00 | 15.83 |
| 1168 | C | SER | A | 332 | −6.506 | −4.147 | −1.548 | 1.00 | 12.57 |
| 1169 | O | SER | A | 332 | −5.710 | −4.987 | −1.958 | 1.00 | 11.85 |
| 1170 | N | ALA | A | 333 | −7.724 | −4.461 | −1.115 | 1.00 | 13.37 |
| 1171 | CA | ALA | A | 333 | −8.223 | −5.827 | −1.186 | 1.00 | 12.68 |
| 1172 | CB | ALA | A | 333 | −9.597 | −5.898 | −0.596 | 1.00 | 12.86 |
| 1173 | C | ALA | A | 333 | −8.236 | −6.308 | −2.647 | 1.00 | 15.04 |
| 1174 | O | ALA | A | 333 | −8.082 | −7.509 | −2.925 | 1.00 | 15.25 |
| 1175 | N | ILE | A | 334 | −8.434 | −5.376 | −3.578 | 1.00 | 14.01 |
| 1176 | CA | ILE | A | 334 | −8.450 | −5.708 | −4.987 | 1.00 | 14.07 |
| 1177 | CB | ILE | A | 334 | −9.149 | −4.598 | −5.764 | 1.00 | 13.20 |
| 1178 | CG2 | ILE | A | 334 | −8.953 | −4.760 | −7.223 | 1.00 | 15.19 |
| 1179 | CG1 | ILE | A | 334 | −10.636 | −4.641 | −5.458 | 1.00 | 14.68 |
| 1180 | CD1 | ILE | A | 334 | −11.453 | −3.593 | −6.150 | 1.00 | 13.41 |
| 1181 | C | ILE | A | 334 | −7.022 | −6.007 | −5.520 | 1.00 | 15.78 |
| 1182 | O | ILE | A | 334 | −6.831 | −6.917 | −6.337 | 1.00 | 14.01 |
| 1183 | N | CYS | A | 335 | −6.022 | −5.273 | −5.032 | 1.00 | 16.38 |
| 1184 | CA | CYS | A | 335 | −4.635 | −5.490 | −5.457 | 1.00 | 18.88 |
| 1185 | CB | CYS | A | 335 | −3.700 | −4.394 | −4.918 | 1.00 | 19.50 |
| 1186 | SG | CYS | A | 335 | −4.015 | −2.754 | −5.628 | 1.00 | 25.06 |
| 1187 | C | CYS | A | 335 | −4.110 | −6.843 | −5.008 | 1.00 | 18.00 |
| 1188 | O | CYS | A | 335 | −3.390 | −7.533 | −5.741 | 1.00 | 18.49 |
| 1189 | N | LEU | A | 336 | −4.489 | −7.212 | −3.801 | 1.00 | 18.10 |
| 1190 | CA | LEU | A | 336 | −4.064 | −8.447 | −3.187 | 1.00 | 19.39 |
| 1191 | CB | LEU | A | 336 | −4.320 | −8.350 | −1.698 | 1.00 | 21.55 |
| 1192 | CG | LEU | A | 336 | −3.820 | −9.468 | −0.803 | 1.00 | 26.30 |
| 1193 | CD1 | LEU | A | 336 | −2.322 | −9.368 | −0.642 | 1.00 | 27.11 |
| 1194 | CD2 | LEU | A | 336 | −4.488 | −9.295 | 0.550 | 1.00 | 25.31 |
| 1195 | C | LEU | A | 336 | −4.748 | −9.690 | −3.750 | 1.00 | 19.53 |
| 1196 | O | LEU | A | 336 | −4.086 | −10.688 | −4.073 | 1.00 | 18.48 |
| 1197 | N | ILE | A | 337 | −6.073 | −9.659 | −3.834 | 1.00 | 19.32 |
| 1198 | CA | ILE | A | 337 | −6.807 | −10.811 | −4.359 | 1.00 | 19.91 |
| 1199 | CB | ILE | A | 337 | −8.156 | −10.997 | −3.605 | 1.00 | 19.73 |
| 1200 | CG2 | ILE | A | 337 | −8.926 | −12.207 | −4.130 | 1.00 | 21.03 |
| 1201 | CG1 | ILE | A | 337 | −7.854 | −11.232 | −2.130 | 1.00 | 18.54 |
| 1202 | CD1 | ILE | A | 337 | −9.052 | −11.440 | −1.328 | 1.00 | 21.98 |
| 1203 | C | ILE | A | 337 | −6.935 | −10.668 | −5.888 | 1.00 | 19.55 |
| 1204 | O | ILE | A | 337 | −7.909 | −10.128 | −6.431 | 1.00 | 19.70 |
| 1205 | N | CYS | A | 338 | −5.867 | −11.090 | −6.551 | 1.00 | 19.24 |
| 1206 | CA | CYS | A | 338 | −5.731 | −11.026 | −7.988 | 1.00 | 19.05 |
| 1207 | CB | CYS | A | 338 | −4.568 | −10.136 | −8.322 | 1.00 | 18.09 |
| 1208 | SG | CYS | A | 338 | −4.456 | −9.913 | −10.046 | 1.00 | 24.64 |
| 1209 | C | CYS | A | 338 | −5.416 | −12.420 | −8.453 | 1.00 | 19.13 |
| 1210 | O | CYS | A | 338 | −4.460 | −13.010 | −7.987 | 1.00 | 18.29 |
| 1211 | N | GLY | A | 339 | −6.213 | −12.951 | −9.367 | 1.00 | 21.16 |
| 1212 | CA | GLY | A | 339 | −5.994 | −14.305 | −9.827 | 1.00 | 21.28 |
| 1213 | C | GLY | A | 339 | −4.999 | −14.426 | −10.946 | 1.00 | 22.01 |
| 1214 | O | GLY | A | 339 | −4.726 | −15.516 | −11.433 | 1.00 | 23.62 |
| 1215 | N | ASP | A | 340 | −4.419 | −13.308 | −11.332 | 1.00 | 22.60 |
| 1216 | CA | ASP | A | 340 | −3.465 | −13.326 | −12.416 | 1.00 | 24.56 |
| 1217 | CB | ASP | A | 340 | −3.737 | −12.157 | −13.343 | 1.00 | 26.57 |
| 1218 | CG | ASP | A | 340 | −5.116 | −12.277 | −13.985 | 1.00 | 31.47 |
| 1219 | OD1 | ASP | A | 340 | −5.312 | −13.227 | −14.795 | 1.00 | 32.06 |
| 1220 | OD2 | ASP | A | 340 | −6.030 | −11.485 | −13.630 | 1.00 | 32.04 |
| 1221 | C | ASP | A | 340 | −2.010 | −13.444 | −12.031 | 1.00 | 23.99 |
| 1222 | O | ASP | A | 340 | −1.130 | −13.301 | −12.865 | 1.00 | 23.98 |
| 1223 | N | ARG | A | 341 | −1.765 | −13.794 | −10.777 | 1.00 | 23.91 |
| 1224 | CA | ARG | A | 341 | −0.406 | −13.961 | −10.310 | 1.00 | 24.57 |
| 1225 | CB | ARG | A | 341 | −0.307 | −13.885 | −8.787 | 1.00 | 22.12 |
| 1226 | CG | ARG | A | 341 | −0.986 | −12.700 | −8.157 | 1.00 | 23.69 |
| 1227 | CD | ARG | A | 341 | −0.405 | −11.410 | −8.644 | 1.00 | 23.69 |
| 1228 | NE | ARG | A | 341 | −0.852 | −10.289 | −7.830 | 1.00 | 23.18 |
| 1229 | CZ | ARG | A | 341 | −0.271 | −9.091 | −7.827 | 1.00 | 24.92 |
| 1230 | NH1 | ARG | A | 341 | 0.795 | −8.839 | −8.597 | 1.00 | 24.43 |
| 1231 | NH2 | ARG | A | 341 | −0.770 | −8.134 | −7.067 | 1.00 | 23.98 |
| 1232 | C | ARG | A | 341 | 0.016 | −15.338 | −10.754 | 1.00 | 25.19 |
| 1233 | O | ARG | A | 341 | −0.797 | −16.257 | −10.871 | 1.00 | 23.68 |
| 1234 | N | MET | A | 342 | 1.303 | −15.458 | −11.026 | 1.00 | 28.20 |
| 1235 | CA | MET | A | 342 | 1.897 | −16.712 | −11.441 | 1.00 | 29.89 |
| 1236 | CB | MET | A | 342 | 3.298 | −16.434 | −11.964 | 1.00 | 31.90 |
| 1237 | CG | MET | A | 342 | 3.356 | −15.306 | −12.979 | 1.00 | 34.96 |
| 1238 | SD | MET | A | 342 | 5.057 | −14.882 | −13.280 | 1.00 | 38.16 |
| 1239 | CE | MET | A | 342 | 5.837 | −16.599 | −13.163 | 1.00 | 35.02 |
| 1240 | C | MET | A | 342 | 1.998 | −17.549 | −10.189 | 1.00 | 29.29 |
| 1241 | O | MET | A | 342 | 1.819 | −17.034 | −9.093 | 1.00 | 31.37 |
| 1242 | N | ASP | A | 343 | 2.292 | −18.830 | −10.347 | 1.00 | 30.14 |
| 1243 | CA | ASP | A | 343 | 2.461 | −19.716 | −9.202 | 1.00 | 29.67 |
| 1244 | CB | ASP | A | 343 | 3.553 | −19.176 | −8.253 | 1.00 | 31.20 |
| 1245 | CG | ASP | A | 343 | 4.964 | −19.442 | −8.758 | 1.00 | 32.74 |
| 1246 | OD1 | ASP | A | 343 | 5.760 | −20.023 | −7.982 | 1.00 | 36.46 |
| 1247 | OD2 | ASP | A | 343 | 5.281 | −19.083 | −9.917 | 1.00 | 33.75 |
| 1248 | C | ASP | A | 343 | 1.202 | −19.984 | −8.399 | 1.00 | 28.09 |
| 1249 | O | ASP | A | 343 | 1.280 | −20.610 | −7.344 | 1.00 | 28.84 |
| 1250 | N | LEU | A | 344 | 0.059 | −19.471 | −8.843 | 1.00 | 25.57 |
| 1251 | CA | LEU | A | 344 | −1.181 | −19.714 | −8.136 | 1.00 | 22.21 |
| 1252 | CB | LEU | A | 344 | −2.242 | −18.714 | −8.541 | 1.00 | 22.99 |
| 1253 | CG | LEU | A | 344 | −2.162 | −17.320 | −7.943 | 1.00 | 21.45 |
| 1254 | CD1 | LEU | A | 344 | −3.464 | −16.616 | −8.305 | 1.00 | 19.98 |
| 1255 | CD2 | LEU | A | 344 | −1.996 | −17.397 | −6.426 | 1.00 | 20.99 |
| 1256 | C | LEU | A | 344 | −1.683 | −21.094 | −8.458 | 1.00 | 22.04 |
| 1257 | O | LEU | A | 344 | −1.693 | −21.490 | −9.613 | 1.00 | 22.32 |
| 1258 | N | GLU | A | 345 | −2.113 | −21.811 | −7.430 | 1.00 | 21.27 |
| 1259 | CA | GLU | A | 345 | −2.637 | −23.162 | −7.570 | 1.00 | 22.42 |
| 1260 | CB | GLU | A | 345 | −2.517 | −23.929 | −6.248 | 1.00 | 20.74 |
| 1261 | CG | GLU | A | 345 | −1.130 | −24.139 | −5.727 | 1.00 | 21.34 |
| 1262 | CD | GLU | A | 345 | −1.110 | −24.823 | −4.356 | 1.00 | 22.75 |
| 1263 | OE1 | GLU | A | 345 | −0.090 | −25.459 | −4.020 | 1.00 | 27.68 |
| 1264 | OE2 | GLU | A | 345 | −2.083 | −24.728 | −3.592 | 1.00 | 22.70 |
| 1265 | C | GLU | A | 345 | −4.112 | −23.159 | −7.991 | 1.00 | 23.85 |
| 1266 | O | GLU | A | 345 | −4.566 | −24.074 | −8.688 | 1.00 | 25.61 |
| 1267 | N | GLU | A | 346 | −4.872 | −22.172 | −7.506 | 1.00 | 23.67 |
| 1268 | CA | GLU | A | 346 | −6.297 | −22.051 | −7.824 | 1.00 | 21.44 |
| 1269 | CB | GLU | A | 346 | −7.115 | −22.369 | −6.586 | 1.00 | 23.93 |
| 1270 | CG | GLU | A | 346 | −6.731 | −23.661 | −5.904 | 1.00 | 26.46 |
| 1271 | CD | GLU | A | 346 | −7.759 | −24.079 | −4.886 | 1.00 | 27.98 |
| 1272 | OE1 | GLU | A | 346 | −8.422 | −25.102 | −5.131 | 1.00 | 30.36 |
| 1273 | OE2 | GLU | A | 346 | −7.931 | −23.378 | −3.859 | 1.00 | 29.40 |
| 1274 | C | GLU | A | 346 | −6.621 | −20.632 | −8.245 | 1.00 | 20.32 |
| 1275 | O | GLU | A | 346 | −7.343 | −19.940 | −7.535 | 1.00 | 18.83 |
| 1276 | N | PRO | A | 347 | −6.183 | −20.210 | −9.447 | 1.00 | 19.81 |
| 1277 | CD | PRO | A | 347 | −5.623 | −20.981 | −10.556 | 1.00 | 19.32 |
| 1278 | CA | PRO | A | 347 | −6.444 | −18.854 | −9.912 | 1.00 | 21.05 |
| 1279 | CB | PRO | A | 347 | −5.813 | −18.831 | −11.300 | 1.00 | 20.06 |
| 1280 | CG | PRO | A | 347 | −4.854 | −19.906 | −11.273 | 1.00 | 18.82 |
| 1281 | C | PRO | A | 347 | −7.907 | −18.599 | −10.004 | 1.00 | 22.73 |
| 1282 | O | PRO | A | 347 | −8.349 | −17.509 | −9.693 | 1.00 | 24.63 |
| 1283 | N | GLU | A | 348 | −8.658 | −19.634 | −10.366 | 1.00 | 25.04 |
| 1284 | CA | GLU | A | 348 | −10.105 | −19.536 | −10.507 | 1.00 | 27.41 |
| 1285 | CB | GLU | A | 348 | −10.727 | −20.861 | −10.990 | 1.00 | 28.57 |
| 1286 | CG | GLU | A | 348 | −9.826 | −21.704 | −11.901 | 1.00 | 33.98 |
| 1287 | CD | GLU | A | 348 | −9.147 | −22.891 | −11.181 | 1.00 | 34.30 |
| 1288 | OE1 | GLU | A | 348 | −8.753 | −22.756 | −10.002 | 1.00 | 34.12 |
| 1289 | OE2 | GLU | A | 348 | −9.025 | −23.974 | −11.804 | 1.00 | 36.51 |
| 1290 | C | GLU | A | 348 | −10.762 | −19.126 | −9.197 | 1.00 | 26.86 |
| 1291 | O | GLU | A | 348 | −11.687 | −18.318 | −9.202 | 1.00 | 28.63 |
| 1292 | N | LYS | A | 349 | −10.307 | −19.676 | −8.076 | 1.00 | 25.97 |
| 1293 | CA | LYS | A | 349 | −10.911 | −19.329 | −6.776 | 1.00 | 25.62 |
| 1294 | CB | LYS | A | 349 | −10.398 | −20.237 | −5.647 | 1.00 | 26.61 |
| 1295 | CG | LYS | A | 349 | −11.091 | −20.003 | −4.291 | 1.00 | 28.17 |
| 1296 | CD | LYS | A | 349 | −10.565 | −20.962 | −3.209 | 1.00 | 29.75 |
| 1297 | CE | LYS | A | 349 | −11.466 | −21.020 | −1.958 | 1.00 | 30.29 |
| 1298 | NZ | LYS | A | 349 | −11.193 | −20.025 | −0.857 | 1.00 | 31.95 |
| 1299 | C | LYS | A | 349 | −10.617 | −17.889 | −6.413 | 1.00 | 24.22 |
| 1300 | O | LYS | A | 349 | −11.445 | −17.199 | −5.815 | 1.00 | 24.94 |
| 1301 | N | VAL | A | 350 | −9.428 | −17.436 | −6.790 | 1.00 | 23.42 |
| 1302 | CA | VAL | A | 350 | −9.020 | −16.079 | −6.489 | 1.00 | 19.37 |
| 1303 | CB | VAL | A | 350 | −7.517 | −15.873 | −6.708 | 1.00 | 17.26 |
| 1304 | CG1 | VAL | A | 350 | −7.127 | −14.526 | −6.154 | 1.00 | 17.10 |
| 1305 | CG2 | VAL | A | 350 | −6.715 | −16.984 | −5.997 | 1.00 | 14.99 |
| 1306 | C | VAL | A | 350 | −9.862 | −15.121 | −7.312 | 1.00 | 17.30 |
| 1307 | O | VAL | A | 350 | −10.335 | −14.126 | −6.790 | 1.00 | 16.58 |
| 1308 | N | ASP | A | 351 | −10.117 | −15.452 | −8.570 | 1.00 | 18.10 |
| 1309 | CA | ASP | A | 351 | −10.960 | −14.595 | −9.410 | 1.00 | 20.96 |
| 1310 | CB | ASP | A | 351 | −11.014 | −15.106 | −10.855 | 1.00 | 25.84 |
| 1311 | CG | ASP | A | 351 | −9.711 | −14.860 | −11.615 | 1.00 | 30.06 |
| 1312 | OD1 | ASP | A | 351 | −8.712 | −14.416 | −10.998 | 1.00 | 32.95 |
| 1313 | OD2 | ASP | A | 351 | −9.688 | −15.119 | −12.833 | 1.00 | 33.18 |
| 1314 | C | ASP | A | 351 | −12.380 | −14.457 | −8.883 | 1.00 | 19.72 |
| 1315 | O | ASP | A | 351 | −12.999 | −13.402 | −9.037 | 1.00 | 19.10 |
| 1316 | N | LYS | A | 352 | −12.894 | −15.536 | −8.297 | 1.00 | 20.03 |
| 1317 | CA | LYS | A | 352 | −14.240 | −15.558 | −7.741 | 1.00 | 19.45 |
| 1318 | CB | LYS | A | 352 | −14.695 | −16.982 | −7.410 | 1.00 | 21.58 |
| 1319 | CG | LYS | A | 352 | −15.209 | −17.769 | −8.631 | 1.00 | 26.04 |
| 1320 | CD | LYS | A | 352 | −16.394 | −17.057 | −9.312 | 1.00 | 29.59 |
| 1321 | CE | LYS | A | 352 | −16.941 | −17.844 | −10.526 | 1.00 | 31.85 |
| 1322 | NZ | LYS | A | 352 | −17.951 | −17.067 | −11.361 | 1.00 | 31.59 |
| 1323 | C | LYS | A | 352 | −14.276 | −14.715 | −6.501 | 1.00 | 18.59 |
| 1324 | O | LYS | A | 352 | −15.303 | −14.120 | −6.185 | 1.00 | 17.49 |
| 1325 | N | LEU | A | 353 | −13.157 | −14.671 | −5.783 | 1.00 | 17.89 |
| 1326 | CA | LEU | A | 353 | −13.071 | −13.854 | −4.575 | 1.00 | 18.26 |
| 1327 | CB | LEU | A | 353 | −11.879 | −14.285 | −3.732 | 1.00 | 19.79 |
| 1328 | CG | LEU | A | 353 | −12.035 | −15.625 | −3.050 | 1.00 | 22.37 |
| 1329 | CD1 | LEU | A | 353 | −10.676 | −16.118 | −2.633 | 1.00 | 22.45 |
| 1330 | CD2 | LEU | A | 353 | −12.959 | −15.441 | −1.860 | 1.00 | 21.09 |
| 1331 | C | LEU | A | 353 | −12.931 | −12.372 | −4.914 | 1.00 | 16.10 |
| 1332 | O | LEU | A | 353 | −13.499 | −11.511 | −4.264 | 1.00 | 17.51 |
| 1333 | N | GLN | A | 354 | −12.211 | −12.073 | −5.972 | 1.00 | 15.53 |
| 1334 | CA | GLN | A | 354 | −12.028 | −10.686 | −6.324 | 1.00 | 16.30 |
| 1335 | CB | GLN | A | 354 | −11.011 | −10.546 | −7.423 | 1.00 | 18.24 |
| 1336 | CG | GLN | A | 354 | −10.490 | −9.134 | −7.548 | 1.00 | 20.89 |
| 1337 | CD | GLN | A | 354 | −9.690 | −8.981 | −8.788 | 1.00 | 20.24 |
| 1338 | OE1 | GLN | A | 354 | −10.152 | −9.344 | −9.864 | 1.00 | 20.86 |
| 1339 | NE2 | GLN | A | 354 | −8.453 | −8.520 | −8.650 | 1.00 | 20.85 |
| 1340 | C | GLN | A | 354 | −13.294 | −10.042 | −6.777 | 1.00 | 16.79 |
| 1341 | O | GLN | A | 354 | −13.531 | −8.886 | −6.460 | 1.00 | 15.54 |
| 1342 | N | GLU | A | 355 | −14.091 | −10.786 | −7.544 | 1.00 | 16.68 |
| 1343 | CA | GLU | A | 355 | −15.356 | −10.278 | −8.085 | 1.00 | 17.94 |
| 1344 | CB | GLU | A | 355 | −16.217 | −11.428 | −8.652 | 1.00 | 20.78 |
| 1345 | CG | GLU | A | 355 | −15.669 | −12.105 | −9.922 | 1.00 | 24.97 |
| 1346 | CD | GLU | A | 355 | −16.624 | −13.171 | −10.526 | 1.00 | 26.48 |
| 1347 | OE1 | GLU | A | 355 | −17.825 | −13.230 | −10.175 | 1.00 | 26.49 |
| 1348 | OE2 | GLU | A | 355 | −16.162 | −13.963 | −11.372 | 1.00 | 29.34 |
| 1349 | C | GLU | A | 355 | −16.207 | −9.377 | −7.153 | 1.00 | 15.27 |
| 1350 | O | GLU | A | 355 | −16.493 | −8.235 | −7.498 | 1.00 | 14.44 |
| 1351 | N | PRO | A | 356 | −16.628 | −9.887 | −5.979 | 1.00 | 13.16 |
| 1352 | CD | PRO | A | 356 | −16.451 | −11.225 | −5.408 | 1.00 | 11.51 |
| 1353 | CA | PRO | A | 356 | −17.431 | −9.050 | −5.089 | 1.00 | 12.93 |
| 1354 | CB | PRO | A | 356 | −17.646 | −9.951 | −3.877 | 1.00 | 13.16 |
| 1355 | CG | PRO | A | 356 | −17.557 | −11.299 | −4.424 | 1.00 | 14.47 |
| 1356 | C | PRO | A | 356 | −16.729 | −7.761 | −4.691 | 1.00 | 13.27 |
| 1357 | O | PRO | A | 356 | −17.382 | −6.761 | −4.405 | 1.00 | 12.63 |
| 1358 | N | LEU | A | 357 | −15.400 | −7.795 | −4.637 | 1.00 | 14.63 |
| 1359 | CA | LEU | A | 357 | −14.618 | −6.619 | −4.254 | 1.00 | 13.35 |
| 1360 | CB | LEU | A | 357 | −13.155 | −6.968 | −4.004 | 1.00 | 12.64 |
| 1361 | CG | LEU | A | 357 | −12.840 | −7.943 | −2.867 | 1.00 | 12.80 |
| 1362 | CD1 | LEU | A | 357 | −11.396 | −8.349 | −2.938 | 1.00 | 11.06 |
| 1363 | CD2 | LEU | A | 357 | −13.117 | −7.318 | −1.528 | 1.00 | 13.33 |
| 1364 | C | LEU | A | 357 | −14.730 | −5.573 | −5.344 | 1.00 | 14.30 |
| 1365 | O | LEU | A | 357 | −14.841 | −4.389 | −5.043 | 1.00 | 16.28 |
| 1366 | N | LEU | A | 358 | −14.756 | −5.996 | −6.607 | 1.00 | 13.24 |
| 1367 | CA | LEU | A | 358 | −14.886 | −5.043 | −7.693 | 1.00 | 13.47 |
| 1368 | CB | LEU | A | 358 | −14.718 | −5.699 | −9.054 | 1.00 | 15.18 |
| 1369 | CG | LEU | A | 358 | −13.305 | −6.100 | −9.463 | 1.00 | 18.18 |
| 1370 | CD1 | LEU | A | 358 | −13.306 | −6.601 | −10.894 | 1.00 | 17.71 |
| 1371 | CD2 | LEU | A | 358 | −12.410 | −4.898 | −9.333 | 1.00 | 17.17 |
| 1372 | C | LEU | A | 358 | −16.258 | −4.428 | −7.605 | 1.00 | 13.18 |
| 1373 | O | LEU | A | 358 | −16.415 | −3.231 | −7.785 | 1.00 | 14.40 |
| 1374 | N | GLU | A | 359 | −17.260 | −5.250 | −7.331 | 1.00 | 12.57 |
| 1375 | CA | GLU | A | 359 | −18.624 | −4.767 | −7.200 | 1.00 | 13.28 |
| 1376 | CB | GLU | A | 359 | −19.573 | −5.928 | −6.986 | 1.00 | 15.79 |
| 1377 | CG | GLU | A | 359 | −20.365 | −6.303 | −8.169 | 1.00 | 21.23 |
| 1378 | CD | GLU | A | 359 | −21.319 | −7.407 | −7.837 | 1.00 | 25.40 |
| 1379 | OE1 | GLU | A | 359 | −22.426 | −7.086 | −7.341 | 1.00 | 26.24 |
| 1380 | OE2 | GLU | A | 359 | −20.947 | −8.588 | −8.038 | 1.00 | 27.57 |
| 1381 | C | GLU | A | 359 | −18.807 | −3.824 | −6.030 | 1.00 | 11.49 |
| 1382 | O | GLU | A | 359 | −19.479 | −2.834 | −6.159 | 1.00 | 12.35 |
| 1383 | N | ALA | A | 360 | −18.303 | −4.179 | −4.862 | 1.00 | 11.57 |
| 1384 | CA | ALA | A | 360 | −18.456 | −3.323 | −3.690 | 1.00 | 13.32 |
| 1385 | CB | ALA | A | 360 | −17.789 | −3.950 | −2.471 | 1.00 | 9.99 |
| 1386 | C | ALA | A | 360 | −17.891 | −1.935 | −3.935 | 1.00 | 13.88 |
| 1387 | O | ALA | A | 360 | −18.503 | −0.933 | −3.544 | 1.00 | 15.74 |
| 1388 | N | LEU | A | 361 | −16.727 | −1.868 | −4.586 | 1.00 | 15.15 |
| 1389 | CA | LEU | A | 361 | −16.090 | −0.577 | −4.872 | 1.00 | 13.83 |
| 1390 | CB | LEU | A | 361 | −14.648 | −0.781 | −5.362 | 1.00 | 14.59 |
| 1391 | CG | LEU | A | 361 | −13.865 | 0.514 | −5.634 | 1.00 | 14.93 |
| 1392 | CD1 | LEU | A | 361 | −13.964 | 1.457 | −4.437 | 1.00 | 12.88 |
| 1393 | CD2 | LEU | A | 361 | −12.413 | 0.212 | −5.963 | 1.00 | 14.12 |
| 1394 | C | LEU | A | 361 | −16.872 | 0.311 | −5.852 | 1.00 | 14.73 |
| 1395 | O | LEU | A | 361 | −16.937 | 1.527 | −5.666 | 1.00 | 16.81 |
| 1396 | N | ARG | A | 362 | −17.418 | −0.273 | −6.920 | 1.00 | 14.29 |
| 1397 | CA | ARG | A | 362 | −18.207 | 0.495 | −7.883 | 1.00 | 13.27 |
| 1398 | CB | ARG | A | 362 | −18.558 | −0.331 | −9.139 | 1.00 | 10.91 |
| 1399 | CG | ARG | A | 362 | −19.257 | 0.531 | −10.203 | 1.00 | 13.29 |
| 1400 | CD | ARG | A | 362 | −19.613 | −0.151 | −11.539 | 1.00 | 14.88 |
| 1401 | NE | ARG | A | 362 | −21.047 | −0.012 | −11.847 | 1.00 | 17.88 |
| 1402 | CZ | ARG | A | 362 | −21.556 | 0.753 | −12.804 | 1.00 | 15.28 |
| 1403 | NH1 | ARG | A | 362 | −20.792 | 1.473 | −13.592 | 1.00 | 15.01 |
| 1404 | NH2 | ARG | A | 362 | −22.856 | 0.812 | −12.955 | 1.00 | 19.91 |
| 1405 | C | ARG | A | 362 | −19.482 | 1.033 | −7.197 | 1.00 | 14.05 |
| 1406 | O | ARG | A | 362 | −19.781 | 2.220 | −7.302 | 1.00 | 13.80 |
| 1407 | N | LEU | A | 363 | −20.189 | 0.173 | −6.463 | 1.00 | 11.82 |
| 1408 | CA | LEU | A | 363 | −21.393 | 0.572 | −5.748 | 1.00 | 12.71 |
| 1409 | CB | LEU | A | 363 | −21.974 | −0.603 | −4.946 | 1.00 | 13.78 |
| 1410 | CG | LEU | A | 363 | −23.326 | −0.466 | −4.220 | 1.00 | 14.50 |
| 1411 | CD1 | LEU | A | 363 | −24.463 | −0.468 | −5.219 | 1.00 | 17.78 |
| 1412 | CD2 | LEU | A | 363 | −23.551 | −1.630 | −3.300 | 1.00 | 13.85 |
| 1413 | C | LEU | A | 363 | −21.051 | 1.663 | −4.763 | 1.00 | 12.77 |
| 1414 | O | LEU | A | 363 | −21.666 | 2.723 | −4.752 | 1.00 | 13.96 |
| 1415 | N | TYR | A | 364 | −20.076 | 1.394 | −3.906 | 1.00 | 13.26 |
| 1416 | CA | TYR | A | 364 | −19.711 | 2.364 | −2.910 | 1.00 | 11.50 |
| 1417 | CB | TYR | A | 364 | −18.690 | 1.782 | −1.951 | 1.00 | 11.85 |
| 1418 | CG | TYR | A | 364 | −18.336 | 2.740 | −0.849 | 1.00 | 12.80 |
| 1419 | CD1 | TYR | A | 364 | −17.235 | 3.582 | −0.955 | 1.00 | 12.10 |
| 1420 | CE1 | TYR | A | 364 | −16.937 | 4.501 | 0.042 | 1.00 | 15.58 |
| 1421 | CD2 | TYR | A | 364 | −19.130 | 2.840 | 0.292 | 1.00 | 13.75 |
| 1422 | CE2 | TYR | A | 364 | −18.837 | 3.751 | 1.293 | 1.00 | 14.21 |
| 1423 | CZ | TYR | A | 364 | −17.742 | 4.580 | 1.164 | 1.00 | 15.51 |
| 1424 | OH | TYR | A | 364 | −17.452 | 5.489 | 2.159 | 1.00 | 16.22 |
| 1425 | C | TYR | A | 364 | −19.207 | 3.662 | −3.520 | 1.00 | 13.59 |
| 1426 | O | TYR | A | 364 | −19.641 | 4.742 | −3.127 | 1.00 | 13.14 |
| 1427 | N | ALA | A | 365 | −18.344 | 3.573 | −4.523 | 1.00 | 13.63 |
| 1428 | CA | ALA | A | 365 | −17.776 | 4.777 | −5.129 | 1.00 | 14.25 |
| 1429 | CB | ALA | A | 365 | −16.678 | 4.386 | −6.123 | 1.00 | 13.92 |
| 1430 | C | ALA | A | 365 | −18.819 | 5.670 | −5.804 | 1.00 | 15.83 |
| 1431 | O | ALA | A | 365 | −18.754 | 6.906 | −5.718 | 1.00 | 15.28 |
| 1432 | N | ARG | A | 366 | −19.766 | 5.030 | −6.488 | 1.00 | 15.42 |
| 1433 | CA | ARG | A | 366 | −20.821 | 5.725 | −7.197 | 1.00 | 16.12 |
| 1434 | CB | ARG | A | 366 | −21.476 | 4.802 | −8.218 | 1.00 | 14.24 |
| 1435 | CG | ARG | A | 366 | −20.693 | 4.672 | −9.492 | 1.00 | 15.76 |
| 1436 | CD | ARG | A | 366 | −21.317 | 3.702 | −10.442 | 1.00 | 18.74 |
| 1437 | NE | ARG | A | 366 | −22.579 | 4.166 | −11.029 | 1.00 | 21.19 |
| 1438 | CZ | ARG | A | 366 | −22.690 | 4.743 | −12.223 | 1.00 | 21.09 |
| 1439 | NH1 | ARG | A | 366 | −21.619 | 4.960 | −12.975 | 1.00 | 22.65 |
| 1440 | NH2 | ARG | A | 366 | −23.889 | 5.049 | −12.691 | 1.00 | 20.53 |
| 1441 | C | ARG | A | 366 | −21.878 | 6.287 | −6.264 | 1.00 | 17.46 |
| 1442 | O | ARG | A | 366 | −22.499 | 7.301 | −6.569 | 1.00 | 18.75 |
| 1443 | N | ARG | A | 367 | −22.127 | 5.598 | −5.161 | 1.00 | 17.91 |
| 1444 | CA | ARG | A | 367 | −23.121 | 6.042 | −4.201 | 1.00 | 17.71 |
| 1445 | CB | ARG | A | 367 | −23.335 | 4.950 | −3.158 | 1.00 | 17.43 |
| 1446 | CG | ARG | A | 367 | −24.125 | 5.398 | −1.959 | 1.00 | 18.44 |
| 1447 | CD | ARG | A | 367 | −24.749 | 4.220 | −1.291 | 1.00 | 16.15 |
| 1448 | NE | ARG | A | 367 | −23.772 | 3.378 | −0.638 | 1.00 | 19.45 |
| 1449 | CZ | ARG | A | 367 | −23.881 | 2.055 | −0.557 | 1.00 | 21.49 |
| 1450 | NH1 | ARG | A | 367 | −24.930 | 1.430 | −1.101 | 1.00 | 21.68 |
| 1451 | NH2 | ARG | A | 367 | −22.951 | 1.354 | 0.081 | 1.00 | 19.07 |
| 1452 | C | ARG | A | 367 | −22.619 | 7.311 | −3.534 | 1.00 | 18.59 |
| 1453 | O | ARG | A | 367 | −23.312 | 8.329 | −3.467 | 1.00 | 20.74 |
| 1454 | N | ARG | A | 368 | −21.363 | 7.268 | −3.119 | 1.00 | 18.65 |
| 1455 | CA | ARG | A | 368 | −20.736 | 8.373 | −2.432 | 1.00 | 17.71 |
| 1456 | CB | ARG | A | 368 | −19.380 | 7.901 | −1.930 | 1.00 | 18.88 |
| 1457 | CG | ARG | A | 368 | −18.756 | 8.689 | −0.815 | 1.00 | 20.07 |
| 1458 | CD | ARG | A | 368 | −17.317 | 8.249 | −0.655 | 1.00 | 22.34 |
| 1459 | NE | ARG | A | 368 | −16.541 | 9.337 | −0.093 | 1.00 | 25.45 |
| 1460 | CZ | ARG | A | 368 | −15.396 | 9.791 | −0.580 | 1.00 | 24.10 |
| 1461 | NH1 | ARG | A | 368 | −14.838 | 9.248 | −1.650 | 1.00 | 20.76 |
| 1462 | NH2 | ARG | A | 368 | −14.869 | 10.874 | −0.037 | 1.00 | 27.70 |
| 1463 | C | ARG | A | 368 | −20.585 | 9.607 | −3.326 | 1.00 | 19.09 |
| 1464 | O | ARG | A | 368 | −20.765 | 10.740 | −2.856 | 1.00 | 21.65 |
| 1465 | N | ARG | A | 369 | −20.248 | 9.399 | −4.605 | 1.00 | 18.12 |
| 1466 | CA | ARG | A | 369 | −20.035 | 10.499 | −5.557 | 1.00 | 17.85 |
| 1467 | CB | ARG | A | 369 | −18.552 | 10.745 | −5.768 | 1.00 | 15.91 |
| 1468 | CG | ARG | A | 369 | −17.929 | 11.408 | −4.596 | 1.00 | 20.93 |
| 1469 | CD | ARG | A | 369 | −16.478 | 11.608 | −4.843 | 1.00 | 25.15 |
| 1470 | NE | ARG | A | 369 | −15.753 | 10.347 | −4.897 | 1.00 | 27.05 |
| 1471 | CZ | ARG | A | 369 | −14.428 | 10.267 | −4.886 | 1.00 | 27.25 |
| 1472 | NH1 | ARG | A | 369 | −13.714 | 11.386 | −4.831 | 1.00 | 24.07 |
| 1473 | NH2 | ARG | A | 369 | −13.822 | 9.078 | −4.884 | 1.00 | 26.35 |
| 1474 | C | ARG | A | 369 | −20.666 | 10.237 | −6.893 | 1.00 | 17.61 |
| 1475 | O | ARG | A | 369 | −19.982 | 9.958 | −7.876 | 1.00 | 18.67 |
| 1476 | N | PRO | A | 370 | −21.982 | 10.373 | −6.967 | 1.00 | 19.44 |
| 1477 | CD | PRO | A | 370 | −22.940 | 10.698 | −5.900 | 1.00 | 18.51 |
| 1478 | CA | PRO | A | 370 | −22.663 | 10.127 | −8.236 | 1.00 | 21.92 |
| 1479 | CB | PRO | A | 370 | −24.137 | 10.116 | −7.832 | 1.00 | 21.07 |
| 1480 | CG | PRO | A | 370 | −24.175 | 11.062 | −6.690 | 1.00 | 20.65 |
| 1481 | C | PRO | A | 370 | −22.365 | 11.231 | −9.229 | 1.00 | 23.55 |
| 1482 | O | PRO | A | 370 | −22.608 | 11.074 | −10.416 | 1.00 | 26.73 |
| 1483 | N | SER | A | 371 | −21.823 | 12.336 | −8.727 | 1.00 | 25.11 |
| 1484 | CA | SER | A | 371 | −21.484 | 13.520 | −9.528 | 1.00 | 25.30 |
| 1485 | CB | SER | A | 371 | −21.121 | 14.685 | −8.578 | 1.00 | 26.75 |
| 1486 | OG | SER | A | 371 | −20.649 | 15.845 | −9.261 | 1.00 | 30.80 |
| 1487 | C | SER | A | 371 | −20.329 | 13.265 | −10.472 | 1.00 | 23.45 |
| 1488 | O | SER | A | 371 | −20.108 | 14.028 | −11.403 | 1.00 | 23.59 |
| 1489 | N | GLN | A | 372 | −19.569 | 12.214 | −10.187 | 1.00 | 22.79 |
| 1490 | CA | GLN | A | 372 | −18.393 | 11.869 | −10.970 | 1.00 | 22.67 |
| 1491 | CB | GLN | A | 372 | −17.132 | 12.276 | −10.209 | 1.00 | 24.31 |
| 1492 | CG | GLN | A | 372 | −17.377 | 12.709 | −8.784 | 1.00 | 26.56 |
| 1493 | CD | GLN | A | 372 | −16.694 | 14.015 | −8.465 | 1.00 | 28.02 |
| 1494 | OE1 | GLN | A | 372 | −16.199 | 14.705 | −9.358 | 1.00 | 28.95 |
| 1495 | NE2 | GLN | A | 372 | −16.660 | 14.365 | −7.189 | 1.00 | 26.49 |
| 1496 | C | GLN | A | 372 | −18.335 | 10.395 | −11.235 | 1.00 | 21.84 |
| 1497 | O | GLN | A | 372 | −17.688 | 9.657 | −10.501 | 1.00 | 22.77 |
| 1498 | N | PRO | A | 373 | −18.983 | 9.942 | −12.308 | 1.00 | 20.20 |
| 1499 | CD | PRO | A | 373 | −19.885 | 10.662 | −13.221 | 1.00 | 20.33 |
| 1500 | CA | PRO | A | 373 | −18.961 | 8.514 | −12.599 | 1.00 | 19.96 |
| 1501 | CB | PRO | A | 373 | −20.107 | 8.353 | −13.598 | 1.00 | 19.66 |
| 1502 | CG | PRO | A | 373 | −20.089 | 9.650 | −14.330 | 1.00 | 19.46 |
| 1503 | C | PRO | A | 373 | −17.629 | 7.918 | −13.078 | 1.00 | 20.26 |
| 1504 | O | PRO | A | 373 | −17.522 | 6.697 | −13.212 | 1.00 | 21.36 |
| 1505 | N | TYR | A | 374 | −16.603 | 8.740 | −13.299 | 1.00 | 18.89 |
| 1506 | CA | TYR | A | 374 | −15.318 | 8.179 | −13.724 | 1.00 | 17.90 |
| 1507 | CB | TYR | A | 374 | −14.520 | 9.128 | −14.608 | 1.00 | 18.18 |
| 1508 | CG | TYR | A | 374 | −14.912 | 9.124 | −16.066 | 1.00 | 19.54 |
| 1509 | CD1 | TYR | A | 374 | −14.079 | 9.709 | −17.034 | 1.00 | 20.63 |
| 1510 | CE1 | TYR | A | 374 | −14.450 | 9.769 | −18.378 | 1.00 | 18.60 |
| 1511 | CD2 | TYR | A | 374 | −16.131 | 8.588 | −16.485 | 1.00 | 19.14 |
| 1512 | CE2 | TYR | A | 374 | −16.515 | 8.639 | −17.821 | 1.00 | 20.11 |
| 1513 | CZ | TYR | A | 374 | −15.676 | 9.230 | −18.765 | 1.00 | 19.94 |
| 1514 | OH | TYR | A | 374 | −16.071 | 9.277 | −20.095 | 1.00 | 20.85 |
| 1515 | C | TYR | A | 374 | −14.435 | 7.763 | −12.566 | 1.00 | 17.56 |
| 1516 | O | TYR | A | 374 | −13.324 | 7.315 | −12.797 | 1.00 | 19.32 |
| 1517 | N | MET | A | 375 | −14.905 | 7.884 | −11.331 | 1.00 | 16.69 |
| 1518 | CA | MET | A | 375 | −14.079 | 7.510 | −10.191 | 1.00 | 15.81 |
| 1519 | CB | MET | A | 375 | −14.723 | 7.952 | −8.875 | 1.00 | 18.75 |
| 1520 | CG | MET | A | 375 | −13.764 | 8.577 | −7.863 | 1.00 | 20.46 |
| 1521 | SD | MET | A | 375 | −12.641 | 9.746 | −8.620 | 1.00 | 23.72 |
| 1522 | CE | MET | A | 375 | −13.665 | 11.177 | −8.979 | 1.00 | 19.74 |
| 1523 | C | MET | A | 375 | −13.742 | 6.023 | −10.168 | 1.00 | 16.19 |
| 1524 | O | MET | A | 375 | −12.567 | 5.661 | −10.044 | 1.00 | 15.08 |
| 1525 | N | PHE | A | 376 | −14.737 | 5.153 | −10.350 | 1.00 | 14.22 |
| 1526 | CA | PHE | A | 376 | −14.464 | 3.721 | −10.328 | 1.00 | 13.87 |
| 1527 | CB | PHE | A | 376 | −15.750 | 2.908 | −10.462 | 1.00 | 11.39 |
| 1528 | CG | PHE | A | 376 | −15.526 | 1.418 | −10.474 | 1.00 | 12.73 |
| 1529 | CD1 | PHE | A | 376 | −15.875 | 0.660 | −11.580 | 1.00 | 14.54 |
| 1530 | CD2 | PHE | A | 376 | −14.988 | 0.764 | −9.373 | 1.00 | 11.18 |
| 1531 | CE1 | PHE | A | 376 | −15.692 | −0.753 | −11.587 | 1.00 | 13.62 |
| 1532 | CE2 | PHE | A | 376 | −14.803 | −0.632 | −9.368 | 1.00 | 12.48 |
| 1533 | CZ | PHE | A | 376 | −15.159 | −1.390 | −10.476 | 1.00 | 13.31 |
| 1534 | C | PHE | A | 376 | −13.408 | 3.307 | −11.387 | 1.00 | 13.55 |
| 1535 | O | PHE | A | 376 | −12.387 | 2.714 | −11.036 | 1.00 | 13.73 |
| 1536 | N | PRO | A | 377 | −13.626 | 3.622 | −12.686 | 1.00 | 13.63 |
| 1537 | CD | PRO | A | 377 | −14.737 | 4.288 | −13.395 | 1.00 | 13.32 |
| 1538 | CA | PRO | A | 377 | −12.578 | 3.197 | −13.623 | 1.00 | 13.92 |
| 1539 | CB | PRO | A | 377 | −13.147 | 3.583 | −14.986 | 1.00 | 12.78 |
| 1540 | CG | PRO | A | 377 | −14.104 | 4.694 | −14.685 | 1.00 | 13.40 |
| 1541 | C | PRO | A | 377 | −11.239 | 3.884 | −13.338 | 1.00 | 14.19 |
| 1542 | O | PRO | A | 377 | −10.191 | 3.314 | −13.605 | 1.00 | 13.68 |
| 1543 | N | ARG | A | 378 | −11.283 | 5.087 | −12.768 | 1.00 | 15.27 |
| 1544 | CA | ARG | A | 378 | −10.064 | 5.819 | −12.434 | 1.00 | 16.15 |
| 1545 | CB | ARG | A | 378 | −10.359 | 7.257 | −12.037 | 1.00 | 16.03 |
| 1546 | CG | ARG | A | 378 | −10.320 | 8.219 | −13.191 | 1.00 | 18.28 |
| 1547 | CD | ARG | A | 378 | −10.250 | 9.656 | −12.710 | 1.00 | 21.80 |
| 1548 | NE | ARG | A | 378 | −11.568 | 10.272 | −12.736 | 1.00 | 28.10 |
| 1549 | CZ | ARG | A | 378 | −11.832 | 11.528 | −12.366 | 1.00 | 32.50 |
| 1550 | NH1 | ARG | A | 378 | −10.851 | 12.336 | −11.937 | 1.00 | 33.14 |
| 1551 | NH2 | ARG | A | 378 | −13.101 | 11.957 | −12.359 | 1.00 | 33.17 |
| 1552 | C | ARG | A | 378 | −9.305 | 5.127 | −11.320 | 1.00 | 15.75 |
| 1553 | O | ARG | A | 378 | −8.075 | 5.051 | −11.353 | 1.00 | 17.14 |
| 1554 | N | MET | A | 379 | −10.017 | 4.611 | −10.333 | 1.00 | 14.20 |
| 1555 | CA | MET | A | 379 | −9.327 | 3.918 | −9.256 | 1.00 | 13.79 |
| 1556 | CB | MET | A | 379 | −10.266 | 3.624 | −8.110 | 1.00 | 13.15 |
| 1557 | CG | MET | A | 379 | −10.710 | 4.856 | −7.389 | 1.00 | 17.50 |
| 1558 | SD | MET | A | 379 | −11.892 | 4.387 | −6.123 | 1.00 | 24.25 |
| 1559 | CE | MET | A | 379 | −13.367 | 4.407 | −7.010 | 1.00 | 18.04 |
| 1560 | C | MET | A | 379 | −8.745 | 2.629 | −9.796 | 1.00 | 13.02 |
| 1561 | O | MET | A | 379 | −7.642 | 2.238 | −9.443 | 1.00 | 12.63 |
| 1562 | N | LEU | A | 380 | −9.476 | 1.975 | −10.676 | 1.00 | 13.77 |
| 1563 | CA | LEU | A | 380 | −8.993 | 0.736 | −11.233 | 1.00 | 15.71 |
| 1564 | CB | LEU | A | 380 | −10.070 | 0.072 | −12.099 | 1.00 | 17.34 |
| 1565 | CG | LEU | A | 380 | −11.231 | −0.615 | −11.377 | 1.00 | 15.40 |
| 1566 | CD1 | LEU | A | 380 | −11.922 | −1.563 | −12.337 | 1.00 | 13.31 |
| 1567 | CD2 | LEU | A | 380 | −10.721 | −1.378 | −10.168 | 1.00 | 15.23 |
| 1568 | C | LEU | A | 380 | −7.709 | 0.944 | −12.027 | 1.00 | 18.33 |
| 1569 | O | LEU | A | 380 | −6.749 | 0.196 | −11.859 | 1.00 | 19.10 |
| 1570 | N | MET | A | 381 | −7.667 | 1.997 | −12.843 | 1.00 | 19.87 |
| 1571 | CA | MET | A | 381 | −6.487 | 2.304 | −13.644 | 1.00 | 19.87 |
| 1572 | CB | MET | A | 381 | −6.766 | 3.474 | −14.588 | 1.00 | 20.21 |
| 1573 | CG | MET | A | 381 | −7.115 | 3.027 | −15.985 | 1.00 | 21.49 |
| 1574 | SD | MET__ | A | 381 | −7.724 | 4.398 | −16.915 | 1.00 | 26.56 |
| 1575 | CE | MET | A | 381 | −8.374 | 3.539 | −18.385 | 1.00 | 23.61 |
| 1576 | C | MET | A | 381 | −5.285 | 2.598 | −12.767 | 1.00 | 18.73 |
| 1577 | O | MET | A | 381 | −4.169 | 2.315 | −13.160 | 1.00 | 19.04 |
| 1578 | N | LYS | A | 382 | −5.525 | 3.122 | −11.570 | 1.00 | 18.70 |
| 1579 | CA | LYS | A | 382 | −4.477 | 3.431 | −10.596 | 1.00 | 18.90 |
| 1580 | CB | LYS | A | 382 | −5.077 | 4.177 | −9.415 | 1.00 | 17.94 |
| 1581 | CG | LYS | A | 382 | −5.189 | 5.654 | −9.668 | 1.00 | 22.52 |
| 1582 | CD | LYS | A | 382 | −3.796 | 6.299 | −9.682 | 1.00 | 21.59 |
| 1583 | CE | LYS | A | 382 | −3.825 | 7.655 | −10.324 | 1.00 | 20.23 |
| 1584 | NZ | LYS | A | 382 | −3.970 | 7.469 | −11.795 | 1.00 | 25.12 |
| 1585 | C | LYS | A | 382 | −3.748 | 2.201 | −10.091 | 1.00 | 19.60 |
| 1586 | O | LYS | A | 382 | −2.681 | 2.312 | −9.491 | 1.00 | 20.41 |
| 1587 | N | ILE | A | 383 | −4.352 | 1.031 | −10.294 | 1.00 | 22.00 |
| 1588 | CA | ILE | A | 383 | −3.773 | −0.250 | −9.882 | 1.00 | 21.94 |
| 1589 | CB | ILE | A | 383 | −4.784 | −1.419 | −10.084 | 1.00 | 23.29 |
| 1590 | CG2 | ILE | A | 383 | −4.073 | −2.772 | −10.000 | 1.00 | 23.62 |
| 1591 | CG1 | ILE | A | 383 | −5.917 | −1.351 | −9.048 | 1.00 | 23.98 |
| 1592 | CD1 | ILE | A | 383 | −6.944 | −2.494 | −9.167 | 1.00 | 22.99 |
| 1593 | C | ILE | A | 383 | −2.576 | −0.487 | −10.790 | 1.00 | 22.86 |
| 1594 | O | ILE | A | 383 | −1.527 | −0.967 | −10.362 | 1.00 | 23.21 |
| 1595 | N | THR | A | 384 | −2.752 | −0.141 | −12.056 | 1.00 | 22.20 |
| 1596 | CA | THR | A | 384 | −1.711 | −0.280 | −13.045 | 1.00 | 22.77 |
| 1597 | CB | THR | A | 384 | −2.285 | −0.035 | −14.462 | 1.00 | 25.21 |
| 1598 | OG1 | THR | A | 384 | −3.222 | −1.089 | −14.759 | 1.00 | 29.09 |
| 1599 | CG2 | THR | A | 384 | −1.189 | −0.031 | −15.517 | 1.00 | 27.05 |
| 1600 | C | THR | A | 384 | −0.548 | 0.639 | −12.712 | 1.00 | 20.89 |
| 1601 | O | THR | A | 384 | 0.598 | 0.195 | −12.707 | 1.00 | 22.32 |
| 1602 | N | ASP | A | 385 | −0.833 | 1.877 | −12.315 | 1.00 | 19.55 |
| 1603 | CA | ASP | A | 385 | 0.239 | 2.811 | −11.961 | 1.00 | 19.49 |
| 1604 | CB | ASP | A | 385 | −0.306 | 4.170 | −11.546 | 1.00 | 20.91 |
| 1605 | CG | ASP | A | 385 | −1.195 | 4.788 | −12.575 | 1.00 | 22.67 |
| 1606 | OD1 | ASP | A | 385 | −1.864 | 5.766 | −12.208 | 1.00 | 24.97 |
| 1607 | OD2 | ASP | A | 385 | −1.230 | 4.326 | −13.733 | 1.00 | 23.80 |
| 1608 | C | ASP | A | 385 | 1.025 | 2.276 | −10.778 | 1.00 | 19.01 |
| 1609 | O | ASP | A | 385 | 2.255 | 2.278 | −10.778 | 1.00 | 18.96 |
| 1610 | N | LEU | A | 386 | 0.289 | 1.884 | −9.743 | 1.00 | 19.05 |
| 1611 | CA | LEU | A | 386 | 0.854 | 1.338 | −8.519 | 1.00 | 18.85 |
| 1612 | CB | LEU | A | 386 | −0.296 | 0.983 | −7.590 | 1.00 | 19.37 |
| 1613 | CG | LEU | A | 386 | −0.096 | 0.569 | −6.138 | 1.00 | 21.27 |
| 1614 | CD1 | LEU | A | 386 | 0.644 | 1.636 | −5.344 | 1.00 | 19.99 |
| 1615 | CD2 | LEU | A | 386 | −1.475 | 0.304 | −5.541 | 1.00 | 20.99 |
| 1616 | C | LEU | A | 386 | 1.759 | 0.125 | −8.787 | 1.00 | 19.60 |
| 1617 | O | LEU | A | 386 | 2.811 | −0.037 | −8.167 | 1.00 | 20.22 |
| 1618 | N | ARG | A | 387 | 1.373 | −0.726 | −9.724 | 1.00 | 20.25 |
| 1619 | CA | ARG | A | 387 | 2.193 | −1.879 | −10.026 | 1.00 | 20.24 |
| 1620 | CB | ARG | A | 387 | 1.397 | −2.848 | −10.888 | 1.00 | 23.00 |
| 1621 | CG | ARG | A | 387 | 2.009 | −4.221 | −11.044 | 1.00 | 28.55 |
| 1622 | CD | ARG | A | 387 | 1.746 | −4.736 | −12.457 | 1.00 | 32.13 |
| 1623 | NE | ARG | A | 387 | 1.966 | −3.644 | −13.412 | 1.00 | 34.51 |
| 1624 | CZ | ARG | A | 387 | 1.421 | −3.570 | −14.622 | 1.00 | 35.87 |
| 1625 | NH1 | ARG | A | 387 | 0.633 | −4.546 | −15.056 | 1.00 | 36.39 |
| 1626 | NH2 | ARG | A | 387 | 1.554 | −2.457 | −15.343 | 1.00 | 36.86 |
| 1627 | C | ARG | A | 387 | 3.486 | −1.392 | −10.715 | 1.00 | 19.97 |
| 1628 | O | ARG | A | 387 | 4.521 | −2.029 | −10.607 | 1.00 | 18.77 |
| 1629 | N | GLY | A | 388 | 3.447 | −0.232 | −11.369 | 1.00 | 18.70 |
| 1630 | CA | GLY | A | 388 | 4.650 | 0.299 | −12.008 | 1.00 | 18.83 |
| 1631 | C | GLY | A | 388 | 5.674 | 0.921 | −11.037 | 1.00 | 20.22 |
| 1632 | O | GLY | A | 388 | 6.893 | 0.833 | −11.248 | 1.00 | 15.88 |
| 1633 | N | ILE | A | 389 | 5.179 | 1.591 | −9.991 | 1.00 | 21.33 |
| 1634 | CA | ILE | A | 389 | 6.032 | 2.221 | −8.970 | 1.00 | 21.20 |
| 1635 | CB | ILE | A | 389 | 5.229 | 3.280 | −8.088 | 1.00 | 22.81 |
| 1636 | CG2 | ILE | A | 389 | 5.126 | 2.852 | −6.639 | 1.00 | 23.64 |
| 1637 | CG1 | ILE | A | 389 | 5.883 | 4.666 | −8.150 | 1.00 | 22.25 |
| 1638 | CD1 | ILE | A | 389 | 5.524 | 5.427 | −9.378 | 1.00 | 21.83 |
| 1639 | C | ILE | A | 389 | 6.623 | 1.129 | −8.083 | 1.00 | 19.67 |
| 1640 | O | ILE | A | 389 | 7.778 | 1.220 | −7.705 | 1.00 | 19.23 |
| 1641 | N | SER | A | 390 | 5.848 | 0.082 | −7.781 | 1.00 | 19.25 |
| 1642 | CA | SER | A | 390 | 6.333 | −1.009 | −6.935 | 1.00 | 19.04 |
| 1643 | CB | SER | A | 390 | 5.183 | −1.882 | −6.477 | 1.00 | 20.61 |
| 1644 | OG | SER | A | 390 | 4.339 | −2.156 | −7.562 | 1.00 | 26.93 |
| 1645 | C | SER | A | 390 | 7.407 | −1.848 | −7.612 | 1.00 | 18.57 |
| 1646 | O | SER | A | 390 | 8.300 | −2.389 | −6.946 | 1.00 | 16.78 |
| 1647 | N | THR | A | 391 | 7.326 | −1.960 | −8.931 | 1.00 | 18.45 |
| 1648 | CA | THR | A | 391 | 8.335 | −2.696 | −9.666 | 1.00 | 20.13 |
| 1649 | CB | THR | A | 391 | 7.930 | −2.914 | −11.066 | 1.00 | 21.93 |
| 1650 | OG1 | THR | A | 391 | 7.487 | −1.673 | −11.621 | 1.00 | 28.62 |
| 1651 | CG2 | THR | A | 391 | 6.806 | −3.897 | −11.099 | 1.00 | 25.50 |
| 1652 | C | THR | A | 391 | 9.587 | −1.863 | −9.624 | 1.00 | 20.07 |
| 1653 | O | THR | A | 391 | 10.685 | −2.401 | −9.524 | 1.00 | 20.66 |
| 1654 | N | LYS | A | 392 | 9.431 | −0.543 | −9.669 | 1.00 | 19.69 |
| 1655 | CA | LYS | A | 392 | 10.587 | 0.332 | −9.550 | 1.00 | 20.05 |
| 1656 | CB | LYS | A | 392 | 10.250 | 1.755 | −9.985 | 1.00 | 20.70 |
| 1657 | CG | LYS | A | 392 | 10.362 | 1.951 | −11.486 | 1.00 | 22.58 |
| 1658 | CD | LYS | A | 392 | 9.843 | 3.311 | −11.846 | 1.00 | 24.75 |
| 1659 | CE | LYS | A | 392 | 9.289 | 3.374 | −13.257 | 1.00 | 26.63 |
| 1660 | NZ | LYS | A | 392 | 8.295 | 4.511 | −13.315 | 1.00 | 28.55 |
| 1661 | C | LYS | A | 392 | 11.098 | 0.282 | −8.094 | 1.00 | 20.04 |
| 1662 | O | LYS | A | 392 | 12.300 | 0.422 | −7.835 | 1.00 | 20.48 |
| 1663 | N | GLY | A | 393 | 10.186 | 0.052 | −7.151 | 1.00 | 18.91 |
| 1664 | CA | GLY | A | 393 | 10.566 | −0.091 | −5.753 | 1.00 | 17.90 |
| 1665 | C | GLY | A | 393 | 11.427 | −1.338 | −5.601 | 1.00 | 18.64 |
| 1666 | O | GLY | A | 393 | 12.451 | −1.311 | −4.925 | 1.00 | 20.47 |
| 1667 | N | ALA | A | 394 | 11.040 | −2.420 | −6.279 | 1.00 | 19.69 |
| 1668 | CA | ALA | A | 394 | 11.761 | −3.689 | −6.264 | 1.00 | 19.72 |
| 1669 | CB | ALA | A | 394 | 11.011 | −4.696 | −7.105 | 1.00 | 21.54 |
| 1670 | C | ALA | A | 394 | 13.181 | −3.547 | −6.789 | 1.00 | 21.85 |
| 1671 | O | ALA | A | 394 | 14.104 | −4.184 | −6.282 | 1.00 | 22.78 |
| 1672 | N | GLU | A | 395 | 13.337 | −2.763 | −7.859 | 1.00 | 24.10 |
| 1673 | CA | GLU | A | 395 | 14.640 | −2.487 | −8.482 | 1.00 | 24.51 |
| 1674 | CB | GLU | A | 395 | 14.466 | −1.672 | −9.774 | 1.00 | 26.19 |
| 1675 | CG | GLU | A | 395 | 13.910 | −2.494 | −10.949 | 1.00 | 31.31 |
| 1676 | CD | GLU | A | 395 | 13.717 | −1.699 | −12.256 | 1.00 | 33.90 |
| 1677 | OE1 | GLU | A | 395 | 13.984 | −0.470 | −12.282 | 1.00 | 33.65 |
| 1678 | OE2 | GLU | A | 395 | 13.288 | −2.322 | −13.263 | 1.00 | 35.18 |
| 1679 | C | GLU | A | 395 | 15.515 | −1.719 | −7.503 | 1.00 | 23.81 |
| 1680 | O | GLU | A | 395 | 16.715 | −1.919 | −7.422 | 1.00 | 23.93 |
| 1681 | N | ARG | A | 396 | 14.884 | −0.851 | −6.740 | 1.00 | 23.94 |
| 1682 | CA | ARG | A | 396 | 15.574 | −0.056 | −5.747 | 1.00 | 23.55 |
| 1683 | CB | ARG | A | 396 | 14.628 | 1.029 | −5.216 | 1.00 | 23.99 |
| 1684 | CG | ARG | A | 396 | 15.199 | 1.891 | −4.115 | 1.00 | 21.36 |
| 1685 | CD | ARG | A | 396 | 16.298 | 2.745 | −4.634 | 1.00 | 21.46 |
| 1686 | NE | ARG | A | 396 | 16.917 | 3.454 | −3.534 | 1.00 | 24.01 |
| 1687 | CZ | ARG | A | 396 | 18.222 | 3.656 | −3.406 | 1.00 | 23.81 |
| 1688 | NH1 | ARG | A | 396 | 19.078 | 3.195 | −4.309 | 1.00 | 23.29 |
| 1689 | NH2 | ARG | A | 396 | 18.661 | 4.380 | −2.391 | 1.00 | 24.32 |
| 1690 | C | ARG | A | 396 | 16.068 | −0.922 | −4.594 | 1.00 | 23.72 |
| 1691 | O | ARG | A | 396 | 17.049 | −0.590 | −3.947 | 1.00 | 24.55 |
| 1692 | N | ALA | A | 397 | 15.368 | −2.009 | −4.299 | 1.00 | 24.28 |
| 1693 | CA | ALA | A | 397 | 15.783 | −2.872 | −3.205 | 1.00 | 23.74 |
| 1694 | CB | ALA | A | 397 | 14.741 | −3.925 | −2.933 | 1.00 | 24.49 |
| 1695 | C | ALA | A | 397 | 17.108 | −3.507 | −3.576 | 1.00 | 24.26 |
| 1696 | O | ALA | A | 397 | 17.943 | −3.779 | −2.721 | 1.00 | 24.86 |
| 1697 | N | ILE | A | 398 | 17.308 | −3.713 | −4.870 | 1.00 | 26.18 |
| 1698 | CA | ILE | A | 398 | 18.547 | −4.291 | −5.384 | 1.00 | 27.23 |
| 1699 | CB | ILE | A | 398 | 18.516 | −4.412 | −6.927 | 1.00 | 27.41 |
| 1700 | CG2 | ILE | A | 398 | 19.685 | −5.238 | −7.416 | 1.00 | 28.46 |
| 1701 | CG1 | ILE | A | 398 | 17.236 | −5.109 | −7.369 | 1.00 | 28.19 |
| 1702 | CD1 | ILE | A | 398 | 16.999 | −6.382 | −6.637 | 1.00 | 29.70 |
| 1703 | C | ILE | A | 398 | 19.731 | −3.406 | −4.977 | 1.00 | 27.78 |
| 1704 | O | ILE | A | 398 | 20.534 | −3.792 | −4.100 | 1.00 | 29.44 |
| 1705 | N | THR | A | 399 | 19.819 | −2.205 | −5.564 | 1.00 | 27.08 |
| 1706 | CA | THR | A | 399 | 20.908 | −1.293 | −5.246 | 1.00 | 26.37 |
| 1707 | CB | THR | A | 399 | 20.785 | 0.016 | −5.994 | 1.00 | 26.11 |
| 1708 | OG1 | THR | A | 399 | 19.421 | 0.414 | −6.022 | 1.00 | 28.83 |
| 1709 | CG2 | THR | A | 399 | 21.235 | −0.161 | −7.405 | 1.00 | 29.58 |
| 1710 | C | THR | A | 399 | 20.988 | −1.049 | −3.755 | 1.00 | 25.99 |
| 1711 | O | THR | A | 399 | 22.079 | −1.025 | −3.186 | 1.00 | 26.23 |
| 1712 | N | LEU | A | 400 | 19.830 | −0.935 | −3.110 | 1.00 | 27.37 |
| 1713 | CA | LEU | A | 400 | 19.800 | −0.723 | −1.673 | 1.00 | 28.07 |
| 1714 | CB | LEU | A | 400 | 18.373 | −0.640 | −1.149 | 1.00 | 27.98 |
| 1715 | CG | LEU | A | 400 | 17.955 | 0.753 | −0.667 | 1.00 | 28.69 |
| 1716 | CD1 | LEU | A | 400 | 16.547 | 0.702 | −0.104 | 1.00 | 26.86 |
| 1717 | CD2 | LEU | A | 400 | 18.932 | 1.293 | 0.368 | 1.00 | 28.98 |
| 1718 | C | LEU | A | 400 | 20.527 | −1.862 | −0.978 | 1.00 | 28.53 |
| 1719 | O | LEU | A | 400 | 21.290 | −1.630 | −0.047 | 1.00 | 28.85 |
| 1720 | N | LYS | A | 401 | 20.327 | −3.087 | −1.447 | 1.00 | 29.09 |
| 1721 | CA | LYS | A | 401 | 21.000 | −4.220 | −0.839 | 1.00 | 29.71 |
| 1722 | CB | LYS | A | 401 | 20.666 | −5.515 | −1.561 | 1.00 | 29.44 |
| 1723 | CG | LYS | A | 401 | 19.240 | −5.946 | −1.356 | 1.00 | 31.48 |
| 1724 | CD | LYS | A | 401 | 18.938 | −7.290 | −1.989 | 1.00 | 32.04 |
| 1725 | CE | LYS | A | 401 | 17.464 | −7.374 | −2.329 | 1.00 | 32.74 |
| 1726 | NZ | LYS | A | 401 | 17.137 | −6.437 | −3.437 | 1.00 | 34.16 |
| 1727 | C | LYS | A | 401 | 22.494 | −3.980 | −0.852 | 1.00 | 29.99 |
| 1728 | O | LYS | A | 401 | 23.164 | −4.170 | 0.153 | 1.00 | 29.22 |
| 1729 | N | MET | A | 402 | 23.007 | −3.486 | −1.971 | 1.00 | 31.43 |
| 1730 | CA | MET | A | 402 | 24.434 | −3.213 | −2.065 | 1.00 | 31.78 |
| 1731 | CB | MET | A | 402 | 24.795 | −2.716 | −3.463 | 1.00 | 34.68 |
| 1732 | CG | MET | A | 402 | 24.275 | −3.512 | −4.648 | 1.00 | 36.65 |
| 1733 | SD | MET | A | 402 | 24.622 | −2.451 | −6.136 | 1.00 | 42.97 |
| 1734 | CE | MET | A | 402 | 23.219 | −2.873 | −7.264 | 1.00 | 38.36 |
| 1735 | C | MET | A | 402 | 24.849 | −2.125 | −1.057 | 1.00 | 31.44 |
| 1736 | O | MET | A | 402 | 25.951 | −2.187 | −0.496 | 1.00 | 32.49 |
| 1737 | N | GLU | A | 403 | 23.970 | −1.144 | −0.832 | 1.00 | 29.62 |
| 1738 | CA | GLU | A | 403 | 24.246 | −0.011 | 0.058 | 1.00 | 28.02 |
| 1739 | CB | GLU | A | 403 | 23.455 | 1.205 | −0.415 | 1.00 | 29.18 |
| 1740 | CG | GLU | A | 403 | 23.567 | 1.495 | −1.923 | 1.00 | 33.06 |
| 1741 | CD | GLU | A | 403 | 22.618 | 2.607 | −2.397 | 1.00 | 35.19 |
| 1742 | OE1 | GLU | A | 403 | 22.053 | 3.320 | −1.530 | 1.00 | 36.93 |
| 1743 | OE2 | GLU | A | 403 | 22.429 | 2.770 | −3.634 | 1.00 | 35.87 |
| 1744 | C | GLU | A | 403 | 24.098 | −0.145 | 1.592 | 1.00 | 28.19 |
| 1745 | O | GLU | A | 403 | 24.645 | 0.678 | 2.335 | 1.00 | 27.87 |
| 1746 | N | ILE | A | 404 | 23.336 | −1.116 | 2.091 | 1.00 | 27.14 |
| 1747 | CA | ILE | A | 404 | 23.200 | −1.256 | 3.542 | 1.00 | 25.57 |
| 1748 | CB | ILE | A | 404 | 21.799 | −1.798 | 3.970 | 1.00 | 24.57 |
| 1749 | CG2 | ILE | A | 404 | 20.743 | −0.754 | 3.715 | 1.00 | 22.48 |
| 1750 | CG1 | ILE | A | 404 | 21.495 | −3.117 | 3.253 | 1.00 | 25.32 |
| 1751 | CD1 | ILE | A | 404 | 20.172 | −3.770 | 3.626 | 1.00 | 24.30 |
| 1752 | C | ILE | A | 404 | 24.334 | −2.124 | 4.097 | 1.00 | 25.35 |
| 1753 | O | ILE | A | 404 | 24.850 | −3.003 | 3.408 | 1.00 | 23.29 |
| 1754 | N | PRO | A | 405 | 24.710 | −1.904 | 5.370 | 1.00 | 26.12 |
| 1755 | CD | PRO | A | 405 | 24.046 | −0.965 | 6.289 | 1.00 | 26.59 |
| 1756 | CA | PRO | A | 405 | 25.782 | −2.618 | 6.072 | 1.00 | 28.17 |
| 1757 | CB | PRO | A | 405 | 26.115 | −1.661 | 7.203 | 1.00 | 28.59 |
| 1758 | CG | PRO | A | 405 | 24.737 | −1.254 | 7.632 | 1.00 | 28.60 |
| 1759 | C | PRO | A | 405 | 25.365 | −3.949 | 6.648 | 1.00 | 29.91 |
| 1760 | O | PRO | A | 405 | 26.129 | −4.564 | 7.402 | 1.00 | 30.96 |
| 1761 | N | GLY | A | 406 | 24.124 | −4.337 | 6.387 | 1.00 | 30.15 |
| 1762 | CA | GLY | A | 406 | 23.629 | −5.599 | 6.880 | 1.00 | 30.34 |
| 1763 | C | GLY | A | 406 | 22.572 | −6.101 | 5.927 | 1.00 | 30.21 |
| 1764 | O | GLY | A | 406 | 22.231 | −5.395 | 4.973 | 1.00 | 30.98 |
| 1765 | N | PRO | A | 407 | 22.049 | −7.318 | 6.131 | 1.00 | 29.00 |
| 1766 | CD | PRO | A | 407 | 22.190 | −8.214 | 7.299 | 1.00 | 30.16 |
| 1767 | CA | PRO | A | 407 | 21.022 | −7.804 | 5.215 | 1.00 | 27.77 |
| 1768 | CB | PRO | A | 407 | 20.907 | −9.265 | 5.613 | 1.00 | 29.21 |
| 1769 | CG | PRO | A | 407 | 21.015 | −9.169 | 7.136 | 1.00 | 29.55 |
| 1770 | C | PRO | A | 407 | 19.749 | −7.047 | 5.567 | 1.00 | 26.71 |
| 1771 | O | PRO | A | 407 | 19.641 | −6.483 | 6.678 | 1.00 | 27.29 |
| 1772 | N | MET | A | 408 | 18.815 | −6.962 | 4.623 | 1.00 | 24.49 |
| 1773 | CA | MET | A | 408 | 17.553 | −6.288 | 4.913 | 1.00 | 22.84 |
| 1774 | CB | MET | A | 408 | 16.747 | −6.104 | 3.622 | 1.00 | 24.24 |
| 1775 | CG | MET | A | 408 | 17.429 | −5.294 | 2.556 | 1.00 | 23.24 |
| 1776 | SD | MET | A | 408 | 16.179 | −4.677 | 1.436 | 1.00 | 24.38 |
| 1777 | CE | MET | A | 408 | 16.887 | −3.079 | 1.097 | 1.00 | 25.22 |
| 1778 | C | MET | A | 408 | 16.752 | −7.153 | 5.909 | 1.00 | 20.40 |
| 1779 | O | MET | A | 408 | 17.020 | −8.341 | 6.049 | 1.00 | 18.50 |
| 1780 | N | PRO | A | 409 | 15.801 | −6.558 | 6.653 | 1.00 | 19.49 |
| 1781 | CD | PRO | A | 409 | 15.501 | −5.124 | 6.731 | 1.00 | 18.87 |
| 1782 | CA | PRO | A | 409 | 14.982 | −7.300 | 7.616 | 1.00 | 18.15 |
| 1783 | CB | PRO | A | 409 | 13.928 | −6.280 | 7.975 | 1.00 | 19.10 |
| 1784 | CG | PRO | A | 409 | 14.729 | −5.031 | 8.009 | 1.00 | 19.00 |
| 1785 | C | PRO | A | 409 | 14.381 | −8.520 | 6.928 | 1.00 | 17.75 |
| 1786 | O | PRO | A | 409 | 13.974 | −8.448 | 5.770 | 1.00 | 15.42 |
| 1787 | N | PRO | A | 410 | 14.238 | −9.632 | 7.657 | 1.00 | 17.26 |
| 1788 | CD | PRO | A | 410 | 14.390 | −9.731 | 9.120 | 1.00 | 16.92 |
| 1789 | CA | PRO | A | 410 | 13.695 | −10.877 | 7.090 | 1.00 | 17.78 |
| 1790 | CB | PRO | A | 410 | 13.732 | −11.844 | 8.287 | 1.00 | 18.05 |
| 1791 | CG | PRO | A | 410 | 13.532 | −10.922 | 9.468 | 1.00 | 18.52 |
| 1792 | C | PRO | A | 410 | 12.335 | −10.827 | 6.384 | 1.00 | 18.59 |
| 1793 | O | PRO | A | 410 | 12.167 | −11.427 | 5.315 | 1.00 | 19.35 |
| 1794 | N | LEU | A | 411 | 11.365 | −10.124 | 6.957 | 1.00 | 18.39 |
| 1795 | CA | LEU | A | 411 | 10.054 | −10.045 | 6.335 | 1.00 | 17.77 |
| 1796 | CB | LEU | A | 411 | 9.011 | −9.489 | 7.297 | 1.00 | 17.32 |
| 1797 | CG | LEU | A | 411 | 8.751 | −10.391 | 8.510 | 1.00 | 18.93 |
| 1798 | CD1 | LEU | A | 411 | 7.396 | −10.095 | 9.093 | 1.00 | 15.54 |
| 1799 | CD2 | LEU | A | 411 | 8.822 | −11.857 | 8.104 | 1.00 | 19.18 |
| 1800 | C | LEU | A | 411 | 10.112 | −9.237 | 5.065 | 1.00 | 17.72 |
| 1801 | O | LEU | A | 411 | 9.379 | −9.509 | 4.114 | 1.00 | 19.61 |
| 1802 | N | ILE | A | 412 | 11.012 | −8.262 | 5.030 | 1.00 | 17.15 |
| 1803 | CA | ILE | A | 412 | 11.186 | −7.433 | 3.846 | 1.00 | 15.82 |
| 1804 | CB | ILE | A | 412 | 12.084 | −6.227 | 4.169 | 1.00 | 15.58 |
| 1805 | CG2 | ILE | A | 412 | 12.498 | −5.522 | 2.909 | 1.00 | 15.37 |
| 1806 | CG1 | ILE | A | 412 | 11.328 | −5.282 | 5.106 | 1.00 | 13.65 |
| 1807 | CD1 | ILE | A | 412 | 12.070 | −4.078 | 5.484 | 1.00 | 14.79 |
| 1808 | C | ILE | A | 412 | 11.782 | −8.300 | 2.740 | 1.00 | 16.91 |
| 1809 | O | ILE | A | 412 | 11.398 | −8.200 | 1.578 | 1.00 | 17.37 |
| 1810 | N | ARG | A | 413 | 12.678 | −9.205 | 3.112 | 1.00 | 18.84 |
| 1811 | CA | ARG | A | 413 | 13.303 | −10.081 | 2.134 | 1.00 | 19.44 |
| 1812 | CB | ARG | A | 413 | 14.493 | −10.827 | 2.734 | 1.00 | 21.68 |
| 1813 | CG | ARG | A | 413 | 15.609 | −9.923 | 3.211 | 1.00 | 23.67 |
| 1814 | CD | ARG | A | 413 | 16.813 | −10.744 | 3.637 | 1.00 | 24.61 |
| 1815 | NE | ARG | A | 413 | 17.413 | −11.353 | 2.458 | 1.00 | 26.77 |
| 1816 | CZ | ARG | A | 413 | 18.364 | −12.275 | 2.487 | 1.00 | 27.71 |
| 1817 | NH1 | ARG | A | 413 | 18.840 | −12.700 | 3.648 | 1.00 | 25.48 |
| 1818 | NH2 | ARG | A | 413 | 18.821 | −12.786 | 1.347 | 1.00 | 29.94 |
| 1819 | C | ARG | A | 413 | 12.307 | −11.071 | 1.579 | 1.00 | 18.31 |
| 1820 | O | ARG | A | 413 | 12.371 | −11.421 | 0.405 | 1.00 | 19.96 |
| 1821 | N | GLU | A | 414 | 11.422 | −11.562 | 2.427 | 1.00 | 17.94 |
| 1822 | CA | GLU | A | 414 | 10.406 | −12.501 | 1.987 | 1.00 | 18.28 |
| 1823 | CB | GLU | A | 414 | 9.504 | −12.894 | 3.156 | 1.00 | 19.64 |
| 1824 | CG | GLU | A | 414 | 10.039 | −14.034 | 3.995 | 1.00 | 19.89 |
| 1825 | CD | GLU | A | 414 | 10.209 | −15.310 | 3.186 | 1.00 | 21.06 |
| 1826 | OE1 | GLU | A | 414 | 9.301 | −15.648 | 2.385 | 1.00 | 20.02 |
| 1827 | OE2 | GLU | A | 414 | 11.254 | −15.970 | 3.354 | 1.00 | 22.16 |
| 1828 | C | GLU | A | 414 | 9.550 | −11.835 | 0.939 | 1.00 | 19.63 |
| 1829 | O | GLU | A | 414 | 9.261 | −12.406 | −0.098 | 1.00 | 19.16 |
| 1830 | N | MET | A | 415 | 9.170 | −10.593 | 1.209 | 1.00 | 21.32 |
| 1831 | CA | MET | A | 415 | 8.322 | −9.846 | 0.299 | 1.00 | 22.45 |
| 1832 | CB | MET | A | 415 | 7.998 | −8.490 | 0.896 | 1.00 | 21.92 |
| 1833 | CG | MET | A | 415 | 7.351 | −7.563 | −0.103 | 1.00 | 22.95 |
| 1834 | SD | MET | A | 415 | 7.180 | −6.000 | 0.638 | 1.00 | 20.83 |
| 1835 | CE | MET | A | 415 | 8.882 | −5.467 | 0.380 | 1.00 | 18.97 |
| 1836 | C | MET | A | 415 | 8.903 | −9.656 | −1.085 | 1.00 | 23.05 |
| 1837 | O | MET | A | 415 | 8.177 | −9.717 | −2.092 | 1.00 | 22.53 |
| 1838 | N | LEU | A | 416 | 10.210 | −9.395 | −1.125 | 1.00 | 24.28 |
| 1839 | CA | LEU | A | 416 | 10.928 | −9.167 | −2.384 | 1.00 | 24.33 |
| 1840 | CB | LEU | A | 416 | 12.128 | −8.217 | −2.171 | 1.00 | 24.70 |
| 1841 | CG | LEU | A | 416 | 11.855 | −6.810 | −1.591 | 1.00 | 23.04 |
| 1842 | CD1 | LEU | A | 416 | 13.139 | −6.167 | −1.129 | 1.00 | 24.40 |
| 1843 | CD2 | LEU | A | 416 | 11.172 | −5.936 | −2.608 | 1.00 | 23.10 |
| 1844 | C | LEU | A | 416 | 11.347 | −10.440 | −3.127 | 1.00 | 25.18 |
| 1845 | O | LEU | A | 416 | 12.048 | −10.363 | −4.122 | 1.00 | 25.92 |
| 1846 | N | GLU | A | 417 | 11.010 | −11.614 | −2.597 | 1.00 | 27.78 |
| 1847 | CA | GLU | A | 417 | 11.301 | −12.861 | −3.310 | 1.00 | 29.15 |
| 1848 | CB | GLU | A | 417 | 11.736 | −14.029 | −2.393 | 1.00 | 28.98 |
| 1849 | CG | GLU | A | 417 | 12.042 | −15.289 | −3.239 | 1.00 | 31.22 |
| 1850 | CD | GLU | A | 417 | 12.651 | −16.473 | −2.495 | 1.00 | 31.45 |
| 1851 | OE1 | GLU | A | 417 | 11.909 | −17.412 | −2.138 | 1.00 | 33.15 |
| 1852 | OE2 | GLU | A | 417 | 13.883 | −16.507 | −2.327 | 1.00 | 30.59 |
| 1853 | C | GLU | A | 417 | 9.959 | −13.188 | −3.950 | 1.00 | 29.66 |
| 1854 | O | GLU | A | 417 | 9.096 | −13.773 | −3.316 | 1.00 | 30.13 |
| 1855 | N | ASN | A | 418 | 9.767 | −12.772 | −5.194 | 1.00 | 32.47 |
| 1856 | CA | ASN | A | 418 | 8.496 | −13.017 | −5.866 | 1.00 | 34.15 |
| 1857 | CB | ASN | A | 418 | 7.432 | −12.067 | −5.300 | 1.00 | 35.46 |
| 1858 | CG | ASN | A | 418 | 7.710 | −10.605 | −5.629 | 1.00 | 34.92 |
| 1859 | OD1 | ASN | A | 418 | 8.097 | −9.833 | −4.759 | 1.00 | 35.44 |
| 1860 | ND2 | ASN | A | 418 | 7.483 | −10.215 | −6.878 | 1.00 | 35.03 |
| 1861 | C | ASN | A | 418 | 8.570 | −12.814 | −7.381 | 1.00 | 35.01 |
| 1862 | O | ASN | A | 418 | 9.326 | −11.960 | −7.853 | 1.00 | 34.95 |
| 1863 | N | PRO | A | 419 | 7.773 | −13.591 | −8.152 | 1.00 | 34.87 |
| 1864 | CD | PRO | A | 419 | 6.936 | −14.692 | −7.644 | 1.00 | 35.71 |
| 1865 | CA | PRO | A | 419 | 7.697 | −13.533 | −9.615 | 1.00 | 35.66 |
| 1866 | CB | PRO | A | 419 | 6.731 | −14.682 | −9.953 | 1.00 | 35.26 |
| 1867 | CG | PRO | A | 419 | 6.908 | −15.642 | −8.817 | 1.00 | 35.03 |
| 1868 | C | PRO | A | 419 | 7.078 | −12.196 | −10.024 | 1.00 | 36.89 |
| 1869 | OT1 | PRO | A | 419 | 6.055 | −11.826 | −9.383 | 1.00 | 38.65 |
| 1870 | OT2 | PRO | A | 419 | 7.604 | −11.540 | −10.960 | 1.00 | 37.30 |
| 1871 | C1 | REA | A | 500 | 11.617 | 0.766 | −0.974 | 1.00 | 22.98 |
| 1872 | C2 | REA | A | 500 | 12.711 | −0.165 | −1.544 | 1.00 | 23.95 |
| 1873 | C3 | REA | A | 500 | 13.522 | −0.885 | −0.510 | 1.00 | 23.03 |
| 1874 | C4 | REA | A | 500 | 12.706 | −1.723 | 0.439 | 1.00 | 21.73 |
| 1875 | C5 | REA | A | 500 | 11.296 | −1.155 | 0.709 | 1.00 | 22.75 |
| 1876 | C6 | REA | A | 500 | 10.782 | −0.041 | 0.060 | 1.00 | 22.00 |
| 1877 | C7 | REA | A | 500 | 9.422 | 0.493 | 0.254 | 1.00 | 21.60 |
| 1878 | C8 | REA | A | 500 | 8.791 | 0.679 | 1.424 | 1.00 | 20.23 |
| 1879 | C9 | REA | A | 500 | 7.449 | 1.141 | 1.670 | 1.00 | 17.02 |
| 1880 | C10 | REA | A | 500 | 7.099 | 1.229 | 2.962 | 1.00 | 17.58 |
| 1881 | C11 | REA | A | 500 | 5.821 | 1.637 | 3.462 | 1.00 | 18.07 |
| 1882 | C12 | REA | A | 500 | 5.727 | 1.752 | 4.782 | 1.00 | 19.51 |
| 1883 | C13 | REA | A | 500 | 4.536 | 2.128 | 5.483 | 1.00 | 21.00 |
| 1884 | C14 | REA | A | 500 | 4.647 | 2.258 | 6.811 | 1.00 | 21.05 |
| 1885 | C15 | REA | A | 500 | 3.667 | 2.658 | 7.784 | 1.00 | 20.02 |
| 1886 | C16 | REA | A | 500 | 10.701 | 1.240 | −2.118 | 1.00 | 24.14 |
| 1887 | C17 | REA | A | 500 | 12.306 | 1.974 | −0.296 | 1.00 | 21.99 |
| 1888 | C18 | REA | A | 500 | 10.607 | −2.018 | 1.769 | 1.00 | 21.60 |
| 1889 | C19 | REA | A | 500 | 6.509 | 1.505 | 0.524 | 1.00 | 13.38 |
| 1890 | C20 | REA | A | 500 | 3.231 | 2.378 | 4.699 | 1.00 | 22.04 |
| 1891 | O1 | REA | A | 500 | 4.026 | 2.789 | 8.938 | 1.00 | 21.58 |
| 1892 | O2 | REA | A | 500 | 2.508 | 2.885 | 7.461 | 1.00 | 23.03 |

It will be understood that various details of the invention can be changed without departing from the scope of the invention. Furthermore, the foregoing description is for the purpose of illustration only, and not for the purpose of limitation-the invention being defined by the claims.

What is claimed is:

1. A crystalline form comprising a substantially pure LXR ligand binding domain polypeptide and a ligand.

2. The crystalline form of claim 1, wherein the LXR ligand binding domain polypeptide comprises the amino acid sequence of SEQ ID NO: 8.

3. The crystalline form of claim 1, wherein the ligand is N-(2,2,2-trifluoroethyl)-N-[4-(2,2,2-trifluoro-1-hydroxy-1-trifluoromethyl-ethyl)-phenyl]-benzenesulfonamide.

4. The crystalline form of claim 1, wherein the ligand is 24(S), 25-epoxycholesterol.

5. The crystalline form of claim 1, wherein the crystalline form has lattice constants of a=60.25 Å, b=82.45 Å, c=123.18 Å, α=90°, β=90°, γ=90°.

6 The crystalline form of claim 1, wherein the crystalline form is an orthorhombic crystalline form.

7. The crystalline form of claim 1, wherein the crystalline form has a space group of P2₁2₁2₁.

8. The crystalline form of claim 1, wherein the crystalline form contains two LXR ligand binding domain polypeptides in the asymmetric unit.

9. The crystalline form of claim 1, wherein the crystalline form is further characterized by the coordinates corresponding to Table 2.

10. The crystalline form of claim 1, wherein the crystalline form is such that the three-dimensional structure of the crystalline form can be determined to a resolution of about 2.8 Å or better.

11. The crystalline form of claim 1, wherein the crystalline form contains one or more atoms having a atomic weight of 40 grams/mol or greater.

12. A crystalline form comprising a substantially pure LXR ligand binding domain polypeptide, a coactivator polypeptide and a ligand.

13. The crystalline form of claim 12, wherein the LXR ligand binding domain polypeptide comprises the amino acid sequence shown of SEQ ID NO: 8.

14. The crystalline form of claim 12, wherein the ligand is 24(S), 25-epoxycholesterol, and the coactivator polypeptide is a SRC1 fragment.

15. The crystalline form of claim 12, wherein the SRC1 fragment comprises the amino acid sequence of SEQ ID NO: 9.

16. The crystalline form of claim 12, wherein the crystalline form has lattice constants selected from the group consisting of a=71.17 Å, b=120.01 Å, c=147.56 Å, α=90, β=90°, γ=90°.

17. The crystalline form of claim 12, wherein the crystalline form is an orthorhombic crystalline form.

18. The crystalline form of claim 12, wherein the crystalline form has a space group of C222₁.

19. The crystalline form of claim 12, wherein the crystalline form contains two LXR ligand binding domain polypeptides and two coactivator polypeptides in the asymmetric unit.

20. The crystalline form of claim 12, wherein the crystalline form is further characterized by the coordinates corresponding to Table 3.

21. The crystalline form of claim 12, wherein the crystalline form is such that the three-dimensional structure of the crystalline form can be determined to a resolution of about 2.8 Å or better.

22. The crystalline form of claim 12, wherein the crystalline form comprises one or more atoms having a atomic weight of 40 grams/mol or greater.

23. A method for determining the three-dimensional structure of a crystallized LXR ligand binding domain polypeptide in complex with one or more of a ligand and a coactivator polypeptide to a resolution of about 2.8 Å or better, the method comprising:
(a) crystallizing a LXR ligand binding domain polypeptide in complex with one or more of a ligand and a coactivator fragment to form a crystallized complex; and
(b) analyzing the crystallized complex to determine the three-dimensional structure of the LXR ligand binding domain polypeptide in complex with one or more of a ligand and a coactivator polypeptide, whereby the three-dimensional structure of a crystallized LXR ligand binding domain polypeptide in complex with one or more of a ligand and a coactivator polypeptide is determined to a resolution of about 2.8 Å or better.

24. The method of claim 23, wherein the analyzing is by X-ray diffraction.

25. The method of claim 23, wherein the crystallization is accomplished by the hanging drop vapor diffusion method, and wherein the LXR ligand binding domain polypeptide and the ligand are mixed with an equal volume of reservoir.

26. The method of claim 25, wherein the reservoir comprises 10-12% PEG3350-8000 and 0.2 M salt.

27. The method of claim 23, wherein the salt is selected from the group consisting of NaF, KF, NaCl, KCl, Na formate, Na acetate, K acetate, ammonium sulfate and lithium sulfate.

28. The method of claim 23, wherein the LXR ligand binding domain polypeptide comprises the amino acid sequence of SEQ ID NO: 8.

29. The method of claim 23, wherein the ligand is selected from the group consisting of 24(S), 25-epoxycholesterol and N-(2,2,2-trifluoroethyl)-N-[4-(2,2,2-trifluoro-1-hydroxy-1-trifluoromethyl-ethyl)-phenyl]-benzenesulfonamide.

30. The method of claim 23, wherein the coactivator polypeptide is a SRC1 polypeptide.

31. The method of claim 30, wherein the SRC1 polypeptide comprises the sequence of SEQ ID NO: 9.

32. A method of generating a crystalline form comprising a LXR ligand binding domain polypeptide in complex with one or more of a ligand and a coactivator polypeptide, the method comprising:
(a) incubating a solution comprising a LXR ligand binding domain and one or more of a ligand and a coactivator polypeptide with an equal volume of reservoir; and
(b) crystallizing the LXR ligand binding domain polypeptide and one or more of a ligand and a coactivator polypeptide using the hanging drop method, whereby a crystalline form of a LXR ligand binding domain polypeptide in complex with one or more of a ligand and a coactivator polypeptide is generated.

33. The method of claim 32, wherein the LXR ligand binding domain comprises the amino acid sequence of SEQ ID NO: 8.

34. The method of claim 32, wherein the ligand is selected from the group consisting of 24(S), 25-epoxycholesterol and N-(2,2,2-trifluoroethyl)-N-[4-(2,2,2-trifluoro-1-hydroxy-1-trifluoromethyl-ethyl)-phenyl]-benzenesulfonamide.

35. The method of claim 32, wherein the coactivator polypeptide comprises a SRC1 polypeptide.

36. The method of claim 35, wherein the SRC1 polypeptide comprises the sequence of SEQ ID NO: 9.

37. A crystalline form of a LXR ligand binding domain polypeptide in complex with one or more of a ligand and a coactivator polypeptide produced by the method of claim 32.

38. A method of designing a modulator of a NR polypeptide, the method comprising:
(a) designing a potential modulator of a NR polypeptide that will make interactions with amino acids in a ligand binding site of the NR polypeptide, based upon a crystalline structure comprising a LXR ligand binding domain polypeptide in complex with a one or more of a ligand and a coactivator polypeptide;
(b) synthesizing the modulator; and
(c) determining whether the potential modulator modulates the activity of the NR polypeptide, whereby a modulator of a NR polypeptide is designed.

39. The method of claim 38, wherein the ligand is selected from the group consisting of 24(S), 25-epoxycholesterol and N-(2,2,2-trifluoroethyl)-N-[4-(2,2,2-trifluoro-1-hydroxy-1-trifluoromethyl-ethyl)-phenyl]-benzenesulfonamide.

40. The method of claim 38, wherein the coactivator polypeptide comprises a SRC1 polypeptide.

41. The method of claim 38, wherein the SRC1 polypeptide comprises the sequence of SEQ ID NO: 9.

42. The method of claim 38, wherein the NR comprises a LXR.

43. The method of claim 42, wherein the LXR comprises a LXRβ.

44. The method of claim 43, wherein the LXRβ comprises the amino acid sequence of SEQ ID NO: 4.

45. The method of claim 38, wherein the ligand is selected from the group consisting of N-(2,2,2-trifluoroethyl)-N-[4-(2,2,2-trifluoro-1-hydroxy-1-trifluoromethyl-ethyl)-phenyl]-benzenesulfonamide and 24(S), 25-epoxycholesterol.

46. A method of designing a modulator that selectively modulates the activity of a NR polypeptide compared to other polypeptides, the method comprising:
(a) obtaining a crystalline form comprising a LXRβ ligand binding domain polypeptide in complex with one or more of a ligand and a coativator polypeptide;
(b) evaluating the three-dimensional structure of the crystallized LXRβ ligand binding domain polypeptide in complex with one or more of a ligand and a coactivator polypeptide; and
(c) synthesizing a potential modulator based on the three-dimensional structure of the crystallized LXRβ ligand binding domain polypeptide in complex with one or more of a ligand and a coactivator polypeptide, whereby a modulator that selectively modulates the activity of a NR polypeptide compared to other polypeptides is designed.

47. The method of claim 46, wherein the method further comprises contacting a NR ligand binding domain polypeptide with the potential modulator and one or more of a ligand and a coactivator polypeptide; and assaying the NR ligand binding domain polypeptide for binding of the potential modulator, for a change in activity of the NR ligand binding domain polypeptide, or both.

48. The method of claim 46, wherein the LXRβ ligand binding domain polypeptide comprises the amino acid sequence of SEQ ID NO: 8.

49. The method of claim 46, wherein the NR comprises a LXR.

50. The method of claim 49, wherein the LXR comprises LXRβ.

51. The method of claim 46, wherein the crystalline form is an orthorhombic form.

52. The method of claim 46, wherein the crystalline form is further characterized by the atomic structural coordinates of one of Table 2 and Table 3.

53. The method of claim 46, wherein the crystalline form is such that the three-dimensional structure of the crystallized LXRβ ligand binding domain polypeptide in complex with one or more of a ligand and a coactivator polypeptide can be determined to a resolution of about 2.8 Å or better.

54. The method of claim 46, wherein the coactivator polypeptide comprises a SRC1 peptide.

55. Then method of claim 54, wherein the SRC1 peptide comprises the sequence of SEQ ID NO: 9.

56. The method of claim 46, wherein the ligand is selected from the group consisting of N-(2,2,2-trifluoroethyl)-N-[4-(2,2,2-trifluoro-1-hydroxy-1-trifluoromethyl-ethyl)-phenyl]-benzenesulfonamide and 24(S), 25-epoxycholesterol.

57. A method of screening a plurality of compounds for a modulator of a NR ligand binding domain polypeptide, the method comprising:
(a) providing a library of test samples;
(b) contacting a crystalline form comprising a LXR ligand binding domain polypeptide in complex with one or more of a ligand and a coactivator polypeptide with each test sample;
(c) detecting an interaction between a test sample and the crystalline LXR ligand binding domain polypeptide in complex with one or more of a ligand and a coactivator polypeptide;
(d) identifying a test sample that interacts with the crystalline LXR ligand binding domain polypeptide in complex with one or more of a ligand and a coactivator polypeptide; and
(e) isolating a test sample that interacts with the crystalline LXR ligand binding domain polypeptide in complex with one or more of a ligand and a coregulator polypeptide, whereby a plurality of compounds is screened for a modulator of a NR ligand binding domain polypeptide.

58. The method of claim 57, wherein the crystalline form is further characterized by an atomic structural coordinate set selected from the group consisting of Table 2 and Table 3.

59. The method of claim 57, wherein the LXR ligand binding domain polypeptide comprises a LXRβ ligand binding domain polypeptide.

60. The method of claim 59, wherein the LXRβ ligand binding domain polypeptide comprises the amino acid sequence of SEQ ID NO: 8.

61. The method of claim 57, wherein the test samples are bound to a substrate.

62. The method of claim 57, wherein the test samples are synthesized directly on a substrate.

63. The method of claim 57, wherein the coregulator polypeptide comprises a SRC1 polypeptide.

64. The method of claim 63, wherein the SRC1 polypeptide comprises the sequence of SEQ ID NO: 9.

65. The method of claim 57, wherein the ligand is selected from the group consisting of N-(2,2,2-trifluoroethyl)-N-[4-(2,2,2-trifluoro-1-hydroxy-1-trifluoromethyl-ethyl)-phenyl]-benzenesulfonamide and 24(S), 25-epoxycholesterol.

66. A method for identifying a NR modulator, the method comprising:
(a) providing atomic structural coordinates describing a LXR ligand binding domain in complex with one or more of a ligand and a coactivator polypeptide to a computerized modeling system; and
(b) modeling ligands that fit spatially into the binding pocket of the LXR ligand binding domain to thereby identify a NR modulator.

67. The method of claim 66, wherein the method further comprises identifying in an assay for NR-mediated activity a modeled ligand that increases or decreases the activity of the NR.

68. The method of claim 66, wherein the atomic structural coordinates are selected from the group consisting of the coordinates of Table 2 and the coordinates of Table 3.

69. The method of claim 66, wherein the LXR comprises LXRβ.

70. The method of claim 66, wherein the LXRβ ligand binding domain comprises the amino acid sequence of SEQ ID NO: 8.

71. The method of claim 66, wherein the coactivator polypeptide comprises a SRC1 polypeptide.

72. The method of claim 71, wherein the SRC1 polypeptide comprises the sequence of SEQ ID NO: 9.

73. The method of claim 66, wherein the ligand is selected from the group consisting of N-(2,2,2-trifluoroethyl)-N-[4-(2,2,2-trifluoro-1-hydroxy-1-trifluoromethyl-ethyl)-phenyl]-benzenesulfonamide and 24(S), 25-epoxycholesterol.

74. A method of identifying a LXR modulator that selectively modulates the activity of a LXRβ polypeptide compared to other polypeptides, the method comprising:
(a) providing atomic structural coordinates describing a LXRβ ligand binding domain in complex with one or more of a ligand and a coactivator polypeptide to a computerized modeling system; and
(b) modeling a ligand that fits into the binding pocket of a LXRβ ligand binding domain and that interacts with residues of a LXRβ that are conserved among LXR subtypes to thereby identify a LXRβ modulator that selectively modulates the activity of a LXRβ polypeptide compared to other polypeptides.

75. The method of claim 74, wherein the method further comprises identifying in a biological assay for LXRβ activity a modeled ligand that selectively binds to said LXRβ and increases or decreases the activity of said LXRβ.

76. The method of claim 74, wherein the atomic structural coordinates are selected from the group consisting of the coordinates of Table 2 and the coordinates of Table 3.

77. The method of claim 74, wherein the LXRβ ligand binding domain comprises the amino acid sequence shown in SEQ ID NO: 8.

78. The method of claim 74, wherein the coactivator polypeptide comprises a SRC1 polypeptide.

79. The method of claim 78, wherein the SRC1 polypeptide comprises the sequence of SEQ ID NO: 9.

80. The method of claim 74, wherein the ligand is selected from the group consisting of N-(2,2,2-trifluoroethyl)-N-[4-(2,2,2-trifluoro-1-hydroxy-1-trifluoromethyl-ethyl)-phenyl]-benzenesulfonamide and 24(S), 25-epoxycholesterol.

81. A method of designing a modulator of a NR polypeptide, the method comprising:
(a) selecting a candidate NR ligand;
(b) determining which amino acid or amino acids of a NR polypeptide interact with the ligand using a three-dimensional model of a crystallized protein, the model comprising a LXRβ ligand binding domain in complex with one or more of a ligand and a coactivator polypeptide;
(c) identifying in a biological assay for NR activity a degree to which the ligand modulates the activity of the NR polypeptide;
(d) selecting a chemical modification of the ligand wherein the interaction between the amino acids of the NR polypeptide and the ligand is predicted to be modulated by the chemical modification;
(e) synthesizing a ligand having the chemical modified to form a modified ligand;
(f) contacting the modified ligand with the NR polypeptide;
(g) identifying in a biological assay for NR activity a degree to which the modified ligand modulates the biological activity of the NR polypeptide; and
(h) comparing the biological activity of the NR polypeptide in the presence of modified ligand with the biological activity of the NR polypeptide in the presence of the unmodified ligand, whereby a modulator of a NR polypeptide is designed.

82. The method of claim 81, wherein the NR polypeptide is a LXR polypeptide.

83. The method of claim 81, wherein the LXR is selected from the group consisting of a LXRα polypeptide and a LXRβ polypeptide.

84. The method of claim 83, wherein the LXRβ polypeptide has the amino acid sequence shown in SEQ ID NO: 4.

85. The method of claim 81, wherein the LXRA polypeptide comprises the amino acid sequence of SEQ ID NO: 2.

86. The method of claim 81, wherein the three-dimensional model of a crystallized protein is further characterized by atomic structural coordinates selected from the group consisting of the coordinates of Table 2 and the coordinates of Table 3.

87. The method of claim 81, wherein the coactivator polypeptide is a SRC1 polypeptide.

88. The method of claim 87, wherein the SRC1 polypeptide comprises the sequence of SEQ ID NO: 9.

89. The method of claim 81, wherein the ligand is selected from the group consisting of N-(2,2,2-trifluoroethyl)-N-[4-(2,2,2-trifluoro-1-hydroxy-1-trifluoromethyl-ethyl)-phenyl]-benzenesulfonamide and 24(S), 25-epoxycholesterol.

90. The method of claim 81, wherein the method further comprises repeating steps (a) through (f), if the biological activity of the NR polypeptide in the presence of the modified ligand varies from the biological activity of the NR polypeptide in the presence of the unmodified ligand.

91. A method of identifying a LXR modulator that selectively modulates the biological activity of one LXR subtype compared to LXRβ, the method comprising:
(a) providing an atomic structure coordinate set describing a LXRβ ligand binding domain structure in complex with one or more of a ligand and a coactivator polypeptide and at least one other atomic structure coordinate set describing a LXR ligand binding domain, each ligand binding domain comprising a ligand binding site;
(b) comparing the LXR atomic structure coordinate sets to identify at least one difference between the sets;
(c) designing a candidate ligand predicted to interact with the difference of step (b);
(d) synthesizing the candidate ligand; and
(e) testing the synthesized candidate ligand for an ability to selectively modulate a LXR subtype as compared to LXRβ, whereby a LXR modulator that selectively modulates the biological activity of one LXR subtype compared to LXRβ is identified.

92. The method of claim 91, wherein the LXR subtype whose biological activity is to be modulated comprises LXRA.

93. The method of claim 92, wherein the LXRα comprises the amino acid sequence of SEQ ID NO: 2.

94. The method of claim 91, wherein the atomic structure coordinate set describing a LXRβ ligand binding domain structure is selected from the group consisting of the coordinates of Table 2 and the coordinates of Table 3.

95. The method of claim 91, wherein the coactivator polypeptide comprises a SRC1 polypeptide.

96. The method of claim 95, wherein the SRC1 polypeptide comprises the sequence of SEQ ID NO: 9.

97. The method of claim 91, wherein the atomic structure coordinate set further describes an associated ligand.

98. The method of claim 97, wherein the ligand is selected from the group consisting of N-(2,2,2-trifluoroethyl)-N-[4-(2,2,2-trifluoro-1-hydroxy-1-trifluoromethyl-ethyl)-phenyl]-benzenesulfonamide and 24(S), 25-epoxycholesterol.

99. A method of modeling a three-dimensional structure of a target NR in complex with one or more of a ligand and a coactivator polypeptide from a template comprising the X-ray structure of a LXRβ LBD in complex with one or more of a ligand and a coactivator polypeptide, the method comprising:
(a) selecting an X-ray structure of a target NR LBD as a starting model for the target NR LBD;
(b) manipulating the starting model for the target NR as a rigid body to superimpose its backbone atoms onto corresponding backbone atoms of a three-dimensional template structure comprising a LXRβ in complex with one or more of a ligand and a coactivator polypeptide to form a manipulated model;
(c) making a copy of the coactivator peptide from the template structure to form a model of a coactivator polypeptide bound to a template LXRβ;
(d) merging the model of the coactivator polypeptide into the manipulated model to form a modified model;
(e) removing one or more amino acids from the modified model; and
(f) optimizing side-chain conformations, whereby a three-dimensional structure of a target NR in complex with one or more of a ligand and a coactivator polypeptide is modeled from a template comprising the X-ray structure of an NR in complex with one or more of a ligand and a coactivator polypeptide.

100. The method of claim 99, wherein the X-ray structure of a target NR LBD is a structure built by homology modeling

101. The method of claim 99, wherein the LXRβ ligand binding domain comprises the sequence of SEQ ID NO: 8.

102. The method of claim 99, wherein the coactivator polypeptide is a SRC1 polypeptide.

103. The method of claim 102, wherein the SRC1 polypeptide comprises the sequence of SEQ ID NO: 9.

104. The method of claim 99, wherein the three-dimensional template structure is a structure characterized by the coordinates selected from the group consisting of the coordinates of Table 2, the coordinates of Table 3 and the coordinates of Table 4.

105. The method of claim 99, wherein the optimizing comprises varying distance constraints.


SEQUENCE LISTING (10 sequences)

SEQ ID NO: 1 - DNA, 1528 bp, Homo sapiens
  CDS (36)..(1379)
cagtgccttggtaatgaccagggctccagaaagagatgtccttgtggctgggggcccctgtgcctgacattcctcctgactctgcggtggagctgtggaagccaggcgcacaggatgcaagcagccaggcccagggaggcagcagctgcatcctcagagaggaagccaggatgccccactctgctgggggtactgcaggggtggggctggaggctgcagagcccacagccctgctcaccagggcagagcccccttcagaacccacagagatccgtccacaaaagcggaaaaaggggccagcccccaaaatgctggggaacgagctatgcagcgtgtgtggggacaaggcctcgggcttccactacaatgttctgagctgcgagggctgcaagggattcttccgccgcagcgtcatcaagggagcgcactacatctgccacagtggcggccactgccccatggacacctacatgcgtcgcaagtgccaggagtgtcggcttcgcaaatgccgtcaggctggcatgcgggaggagtgtgtcctgtcagaagaacagatccgcctgaagaaactgaagcggcaagaggaggaacaggctcatgccacatccttgccccccaggcgttcctcacccccccaaatcctgccccagctcagcccggaacaactgggcatgatcgagaagctcgtcgctgcccagcaacagtgtaaccggcgctccttttctgaccggcttcgagtcacgccttggcccatggcaccagatccccatagccgggaggcccgtcagcagcgctttgcccacttcactgagctggccatcgtctctgtgcaggagatagttgactttgctaaacagctacccggcttcctgcagctcagccgggaggaccagattgccctgctgaagacctctgcgatcgaggtgatgcttctggagacatctcggaggtacaaccctgggagtgagagtatcaccttcctcaaggatttcagttataaccgggaagactttgccaaagcagggctgcaagtggaattcatcaaccccatcttcgagttctccagggccatgaatgagctgcaactcaatgatgccgagtttgccttgctcattgctatcagcatcttctctgcagaccggcccaacgtgcaggaccagctccaggtggagaggctgcagcacacatatgtggaagccctgcatgcctacgtctccatccaccatccccatgaccgactgatgttcccacggatgctaatgaaactggtgagcctccggaccctgagcagcgtccactcagagcaagtgtttgcactgcgtctgcaggacaaaaagctcccaccgctgctctctgagatctgggatgtgcacgaatgactgttctgtccccatattttctgttttcttggccggatggctgaggcctggtggctgcctcctagaagtggaacagactgagaagggcaaacattcctgggagctgggcaaggagatcctcccgtggcattaaaagagagtcaaagggt

SEQ ID NO: 2 - protein, 447 aa, Homo sapiens
MSLWLGAPVPDIPPDSAVELWKPGAQDASSQAQGGSSCILREEARMPHSAGGTAGVGLEAAEPTALLTRAEPPSEPTEIRPQKRKKGPAPKMLGNELCSVCGDKASGFHYNVLSCEGCKGFFRRSVIKGAHYICHSGGHCPMDTYMRRKCQECRLRKCRQAGMREECVLSEEQIRLKKLKRQEEEQAHATSLPPRRSSPPQILPQLSPEQLGMIEKLVAAQQQCNRRSFSDRLRVTPWPMAPDPHSREARQQRFAHFTELAIVSVQEIVDFAKQLPGFLQLSREDQIALLKTSAIEVMLLETSRRYNPGSESITFLKDFSYNREDFAKAGLQVEFINPIFEFSRAMNELQLNDAEFALLIAISIFSADRPNVQDQLQVERLQHTYVEALHAYVSIHHPHDRLMFPRMLMKLVSLRTLSSVHSEQVFALRLQDKKLPPLLSEIWDVHE

SEQ ID NO: 3 - DNA, 2010 bp, Homo sapiens
  CDS (245)..(1630)
caagaagtggcgaagttacctttgagggtatttgagtagcggcggtgtgtcaggggctaaagaggaggacgaagaaaagcagagcaagggaacccagggcaacaggagtagttcactccgcgagaggccgtccacgagacccccgcgcgcaggcatgagccccgccccccacgcatgagccccgccccccgctgttgcttggagaggggcgggacctggagagaggctgctccgtgaccccaccatgtcctctcctaccacgagttccctggatacccccctgcctggaaatggcccccctcagcctggcgccccttcttcttcacccactgtaaaggaggagggtccggagccgtggcccgggggtccggaccctgatgtcccaggcactgatgaggccagctcagcctgcagcacagactgggtcatcccagatcccgaagaggaaccagagcgcaagcgaaagaagggcccagccccgaagatgctgggccacgagctttgccgtgtctgtggggacaaggcctccggcttccactacaacgtgctcagctgcgaaggctgcaagggcttcttccggcgcagtgtggtccgtggtggggccaggcgctatgcctgccggggtggcggaacctgccagatggacgctttcatgcggcgcaagtgccagcagtgccggctgcgcaagtgcaaggaggcagggatgagggagcagtgcgtcctttctgaagaacagatccggaagaagaagattcggaaacagcagcagcaggagtcacagtcacagtcgcagtcacctgtggggccgcagggcagcagcagctcagcctctgggcctggggcttcccctggtggatctgaggcaggcagccagggctccggggaaggcgagggtgtccagctaacagcggctcaagaactaatgatccagcagttggtggcggcccaactgcagtgcaacaaacgctccttctccgaccagcccaaagtcacgccctggcccctgggcgcagacccccagtcccgagatgcccgccagcaacgctttgcccacttcacggagctggccatcatctcagtccaggagatcgtggacttcgctaagcaagtgcctggtttcctgcagctgggccgggaggaccagatcgccctcctgaaggcatccactatcgagatcatgctgctagagacagccaggcgctacaaccacgagacagagtgtatcaccttcttgaaggacttcacctacagcaaggacgacttccaccgtgcaggcctgcaggtggagttcatcaaccccatcttcgagttctcgcgggccatgcggcggctgggcctggacgacgctgagtacgccctgctcatcgccatcaacatcttctcggccgaccggcccaacgtgcaggagccgggccgcgtggaggcgttgcagcagccctacgtggaggcgctgctgtcctacacgcgcatcaagaggccgcaggaccagctgcgcttcccgcgcatgctcatgaagctggtgagcctgcgcacgctgagctctgtgcactcggagcaggtcttcgccttgcggctccaggacaagaagctgccgcctctgctgtcggagatctgggacgtccacgagtgaggggctggccacccagccccacagccttgcctgaccaccctccagcagatagacgccggcaccccttcctcttcctagggtggaaggggccctgggcgagcctgtagacctatcggctctcatcccttgggataagccccagtccaggtccaggaggctccctccctgcccagcgagtcttccagaaggggtgaaagggttgcaggtcccgaccactgacccttcccggctgccctccctccccagcttacacctcaagcccagcacgcagcgtaccttgaacagagggaggggaggacccatggctctccccccctagcccgggagaccaggggccttcctcttcctctgcttttatttaataaaaataaaaacagaaa

SEQ ID NO: 4 - protein, 461 aa, Homo sapiens
MSSPTTSSLDTPLPGNGPPQPGAPSSSPTVKEEGPEPWPGGPDPDVPGTDEASSACSTDWVIPDPEEEPERKRKKGPAPKMLGHELCRVCGDKASGFHYNVLSCEGCKGFFRRSVVRGGARRYACRGGGTCQMDAFMRRKCQQCRLRKCKEAGMREQCVLSEEQIRKKKIRKQQQQESQSQSQSPVGPQGSSSSASGPGASPGGSEAGSQGSGEGEGVQLTAAQELMIQQLVAAQLQCNKRSFSDQPKVTPWPLGADPQSRDARQQRFAHFTELAIISVQEIVDFAKQVPGFLQLGREDQIALLKASTIEIMLLETARRYNHETECITFLKDFTYSKDDFHRAGLQVEFINPIFEFSRAMRRLGLDDAEYALLIAINIFSADRPNVQEPGRVEALQQPYVEALLSYTRIKRPQDQLRFPRMLMKLVSLRTLSSVHSEQVFALRLQDKKLPPLLSEIWDVHE

SEQ ID NO: 5 - DNA, 660 bp, Homo sapiens
  CDS (1)..(660)
gagaagctcgtcgctgcccagcaacagtgtaaccggcgctccttttctgaccggcttcgagtcacgccttggcccatggcaccagatccccatagccgggaggcccgtcagcagcgctttgcccacttcactgagctggccatcgtctctgtgcaggagatagttgactttgctaaacagctacccggcttcctgcagctcagccgggaggaccagattgccctgctgaagacctctgcgatcgaggtgatgcttctggagacatctcggaggtacaaccctgggagtgagagtatcaccttcctcaaggatttcagttataaccgggaagactttgccaaagcagggctgcaagtggaattcatcaaccccatcttcgagttctccagggccatgaatgagctgcaactcaatgatgccgagtttgccttgctcattgctatcagcatcttctctgcagaccggcccaacgtgcaggaccagctccaggtggagaggctgcagcacacatatgtggaagccctgcatgcctacgtctccatccaccatccccatgaccgactgatgttcccacggatgctaatgaaactggtgagcctccggaccctgagcagcgtccactcagagcaagtgtttgcactgcgtctgcaggacaaaaag

SEQ ID NO: 6 - protein, 220 aa, Homo sapiens
EKLVAAQQQCNRRSFSDRLRVTPWPMAPDPHSREARQQRFAHFTELAIVSVQEIVDFAKQLPGFLQLSREDQIALLKTSAIEVMLLETSRRYNPGSESITFLKDFSYNREDFAKAGLQVEFINPIFEFSRAMNELQLNDAEFALLIAISIFSADRPNVQDQLQVERLQHTYVEALHAYVSIHHPHDRLMFPRMLMKLVSLRTLSSVHSEQVFALRLQDKK

SEQ ID NO: 7 - DNA, 792 bp, Artificial Sequence
  LXR LBD Construct
atgaaaaaacatcatcatcatcatcatctggttccgcgtggatccgaaggcgagggtgtccagctaacagcggctcaagaactaatgatccagcagttggtggcggcccaactgcagtgcaacaaacgctccttctccgaccagcccaaagtcacgccctggcccctgggcgcagacccccagtcccgagatgcccgccagcaacgctttgcccacttcacggagctggccatcatctcagtccaggagatcgtggacttcgctaagcaagtgcctggtttcctgcagctgggccgggaggaccagatcgccctcctgaaggcatccactatcgagatcatgctgctagagacagccaggcgctacaaccacgagacagagtgtatcaccttcttgaaggacttcacctacagcaaggacgacttccaccgtgcaggcctgcaggtggagttcatcaaccccatcttcgagttctcgcgggccatgcggcggctgggcctggacgacgctgagtacgccctgctcatcgccatcaacatcttctcggccgaccggcccaacgtgcaggagccgggccgcgtggaggcgttgcagcagccctacgtggaggcgctgctgtcctacacgcgcatcaagaggccgcaggaccagctgcgcttcccgcgcatgctcatgaagctggtgagcctgcgcacgctgagctctgtgcactcggagcaggtcttcgccttgcggctccaggacaagaagctgccgcctctgctgtcggagatctgggacgtccacgagtag

SEQ ID NO: 8 - protein, 263 aa, Artificial Sequence
  LXR LBD Construct
MKKHHHHHHLVPRGSEGEGVQLTAAQELMIQQLVAAQLQCNKRSFSDQPKVTPWPLGADPQSRDARQQRFAHFTELAIISVQEIVDFAKQVPGFLQLGREDQIALLKASTIEIMLLETARRYNHETECITFLKDFTYSKDDFHRAGLQVEFINPIFEFSRAMRRLGLDDAEYALLIAINIFSADRPNVQEPGRVEALQQPYVEALLSYTRIKRPQDQLRFPRMLMKLVSLRTLSSVHSEQVFALRLQDKKLPPLLSEIWDVHE

SEQ ID NO: 9 - protein, 25 aa, Homo sapiens
CPSSHSSLTERHKILHRLLQQGSPS

SEQ ID NO: 10 - protein, 238 aa, Homo sapiens
LSPALEELITKVSKAHQETFPSLCQLGKYTTNSSADHRVQLDLGLWDKFSELATKCIIKIVEFAKRLPGFTGLSIADQITLLKAACLDILMLRICTRYTPEQDTMTFSDGLTLNRTQMHNAGFGPLTDLVFAFAGQLLPLEMDDTETGLLSAICLICGDRMDLEEPEKVDKLQEPLLEALRLYARRRRPSQPYMFPRMLMKITDLRGISTKGAERAITLKMEIPGPMPPLIREMLENP